(19) United States
(12) Patent Application Publication
(10) Pub. No.: US 2003/0148324 A1
(43) Pub. Date: Aug. 7, 2003

(54) Polynucleotides which are of nature B2/D+ A- and which are isolated from E. coli, and biological uses of these polynucleotides and of their polypeptides

(76) Inventors: Edouard Bingen, Paris (FR); Stephane Bonacorsi, Nogent Sur Marne (FR); Olivier Clermont, Paris (FR); Xavier Nassif, Paris (FR); Colin Tinsley, Paris (FR)

Correspondence Address: NIXON & VANDERHYE, PC, 1100 N GLEBE ROAD, 8TH FLOOR, ARLINGTON, VA, 22201-4714, US

(21) Appl. No.: 10/238,075

(22) Filed: Sep. 10, 2002

Related U.S. Application Data
(63) Continuation-in-part of application No. PCT/EP01/03445, filed on Dec. 3, 2001.

(30) Foreign Application Priority Data
Feb. 2, 2001 (FR) 01 01449

(51) Int. Cl. C12Q 1/68; C07H 21/04; C12N 1/21

(52) U.S. Cl. 435/6; 435/252.33; 536/23.7

(57) ABSTRACT

The present invention relates to products which are of nature B2+ A−, isolated from E. coli, and to their biological applications, in particular their medical (therapeutic, vaccine and diagnostic) and biotechnological applications. In the present application, the expression “of nature B2+ A−” is intended to mean presence at a frequency greater than 10% among the E. coli strains of group B2 of the ECOR collection, and at a frequency of less than 10% among the strains of group A of the same collection. A phylogenic determination method which makes it possible to rapidly and easily distinguish the groups A, B1, B2 and D of the E. coli species with more than 99% precision is in particular described.

The present invention relates, in general, to polynucleotides which are of nature B2/D+ A− and which are isolated from E. coli, and to the biological uses, in particular medical and biotechnological uses, of these polynucleotides and of the polypeptides which they encode. In the present invention, the expression “the nature B2/D+ A−” is intended to mean that the polynucleotide is present with greater frequency such as observed in ECOR E. coli of group B2 (ECOR B2 frequency) and/or in ECOR E. coli of group D (ECOR D frequency), with respect to the frequency observed in ECOR E. coli of group A (ECOR A frequency). Preferably, the ECOR B2 frequency and/or the ECOR D frequency is greater than the ECOR A frequency by a factor of 2, preferably of 3, more preferably of 3.5, very preferably by a factor of 4. The set of polynucleotides which are of nature B2/D+ A−, provided by the present invention, comprises in particular products the ECOR B2 frequency and/or the ECOR D frequency of which is greater than 10%, and the ECOR A frequency of which is less than 25%, preferably less than 20%, more preferably less than 10%, and very preferably less than 5%, while always remaining less than the ECOR B2 frequency and/or the ECOR D frequency.

The ECOR collection is a collection which represents the genetic diversity of the E. coli species; it is available from banks such as the ATCC.

The E. coli species is currently divided into four main phylogenic groups termed groups A, B1, B2 and D. Currently known techniques for determining the phylogenic group of a given E. coli strain comprise multilocus enzymatic electrophoresis, or MLEE, and ribotyping. Descriptions of these techniques are in particular described in Herzer et al. 1990 J. Bacteriol. 172:6175-6181 and in Selander et al. 1986 Appl. Environ. Microbiol. 51:873-884 for MLEE, and in Bingen et al. 1994 Clin. Microbiol. Rev. 7:311-317, Bingen et al. Clin. Infect. Dis. 22:152-156, Bingen et al. J. Infect. Dis. 177:642-650 and in Desjardins et al. 1995 J. Mol. Evol. 41:440-448, for ribotyping. Briefly, MLEE is based on the analysis of the migration polymorphism of bacterial enzymes. For each strain, a large number of enzymes characteristic of the species (often greater than or equal to 20) are characterized by their electrophoretic mobility. The existence of migration variants for these enzymes makes it possible to characterize each strain by an electrophoretic type. With regard to ribotyping, it is based on the analysis of the restriction polymorphism of the chromosomal regions which include the genes encoding the 16S and 23S RNAs. This polymorphism is revealed using a labelled cDNA probe, prepared from the 16S and 23S RNA of E. coli, which can be hybridized to the DNA of the strain studied, after digestion of this DNA with various restriction enzymes and. Southern transfer.

The techniques of the prior art are, however, complicated to carry out for industrial-type applications, such as analyses in a medical environment or large scale screening of strains of interest for biotechnological techniques such as cloning. In addition, these techniques are long and require the availability of a collection of reference strains.

A collection termed ECOR has, moreover, been constituted in such a way as to represent the genetic diversity of the E. coli species. This collection is available in particular from the ATCC (ATCC No. 35320 to No. 35391). It comprises:

25 E. coli strains of group A,

16 E. coli strains of group B1,

15 E. coli strains of group B2,

12 E. coli strains of group D, and

4 E. coli strains not assigned to one of the four groups A, B1, B2 and D.

In order to provide a more effective solution to the problem of the phylogenic determination of a given E. coli strain, the present invention makes the original and pertinent choice of isolating the entire set of its polynucleotides which are of nature B2/D+ A−, as defined above. Such a solution has never, to the Applicant's knowledge, been proposed. Now, the present invention demonstrates that this particular choice not only makes it possible to solve the problem of the phylogenic determination of a given E. coli strain, but also makes it possible to detect and to treat an undesired development of E. coli , and more particularly a development of E. coli in a human or animal compartment which is extra-intestinal (systemic and non-diarrhoeal infections, such as septicaemia, pyelonephritis, or meningitis in the newborn). The present invention in fact provides, in addition to the diagnostic and therapeutic means directed against E. coli in general, specific diagnostic and therapeutic means which have the advantage of distinguishing between E. coli capable of infecting the extra-intestinal compartment (E. coli of group B2 and D) and E. coli which are part of the normal physiological flora of humans and animals. Besides the less deleterious effects that such treatments offer patients suffering from an extra-intestinal E. coli infection, these specific diagnostic methods and treatments according to the invention make it possible to limit the development of bacterial resistances which are more and more frequently observed as broad-spectrum antibiotic treatments are used.

The inventors have therefore developed means which provide access to the entire set of polynucleotides which are of nature B2/D+ A−, as defined above. To the Applicant's knowledge, this is the first description of such means and the first description of such a set of products. These means also demonstrate that there are genomic regions which are of nature B2/D+ A−.

In the scientific literature, a few products have been individually described as being present in one or more E. coli strains of group B2 or D, and as being generally absent from one or more E. coli strains of A. It is in particular the case of the sfa, ibe10, hly, pap, prs and kps genes, of the genes involved in sorbose metabolism. The reliability of these products as phylogenic markers has, however, never been demonstrated. In any event, there is nothing to indicate that these products are capable of effectively distinguishing between the various phylogenic groups of E. coli. In addition, these products have been isolated separately, with the observation of their presence in one or more E. coli strains of group B2 or D, and of their absence in the few E. coli strains of group A which have been tested, being made a posteriori. Those skilled in the art did not, therefore, in the prior art, have means for obtaining other products possibly having the nature B2/D+ A−.

A concept which is common to the various aspects of the invention corresponds to producing phylogenic markers of E. coli by choosing to isolate the set of the chromosomal and plasmid DNA fragments of E. coli which are of nature B2/D+ A− as defined above, and developing, for this purpose, a method which is capable of allowing the entire set of these fragments to be isolated.

The method used consists in subtracting the polynucleotide population of one or more E. coli strain(s) of group A, randomly sheared, from the polynucleotide population of one or more E. coli strain(s) of group B2 or D, cleaved with a restriction endonuclease allowing fragments comprising from 100 to 1 500 bp approximately, with a mean of 300 to 500 bp approximately, to be produced, such as Sau3AI, Tsp509I, MspI or MaeII (any restriction enzyme with short restriction sites, for example for bp). This method makes it possible to obtain a library of products which are of nature B2/D+ A− as defined above. Example 1 below gives an illustration thereof, for which the E. coli strain subjected to subtraction is the E. coli strain C5. This particular strain is classified, according to reference techniques, in the phylogenic group B2, yet it comprises, in particular at the plasmid level, DNAs of D origin. The specific choice of E. coli C5 therefore has the advantage of isolating polynucleotides which are of nature B2+ A− and/or D+ A−, using a single strain.

Such a subtraction can be repeated over and over until the desired level of exhaustiveness is obtained (the level of exhaustiveness of the library can be estimated by searching for the presence or absence of products known to have the nature B2/D+ A−, such as sfa, ibe10). If the intention is to increase the level of exhaustiveness, it is desirable to use different restriction enzymes between the various subtraction repetitions. Examples of implementation of such a method are in particular described in Example 1 which follows.

Various subtractive methods for isolating DNAs present in one bacterium and absent in another bacterium have been described in the prior art. Before the present invention, such a subtractive method had, however, never been applied to the E. coli species, nor had it been envisaged that such a method may provide a solution to the problem of identifying phylogenic markers in general, and to the problem of identifying phylogenic markers of the E. coli species in particular. What is more, the present invention proposes, for the first time, to apply a subtractive method to a specific choice of E. coli strains: firstly, an isolate of E. coli of group B2, associated with neonatal meningitis (E. coli C5 assigned to the phylogenic group B2 and comprising, in particular at the plasmid level, DNAs of D origin), and secondly, E. coli strains of group A from the ECOR collection.

The present invention therefore offers the means for obtaining all of the products which are of nature B2/D+ A−. As such, the polynucleotides targeted by the present application correspond to the set of polynucleotides which are of novel nature B2/D+ A−. Some of these polynucleotides correspond to products which are novel in themselves (SEQ ID NO: 1 to NO: 153, sequences corresponding to regions 1, 3, 4 and 5 identified in the following examples, orf sequences containing these sequences). Others correspond to products which have homologies with known products, but which are of novel nature B2/D+ A− (in particular SEQ ID NO: 170, 171, 174, 175, 178, 179, 183, 185, 186, 190, 191, 193, 194, 195, 196, 199, 200, 202, 205, 206, 208, 209, 211, 214, 218, 220, 221, 233, 234, 235, 241, 244, 246, 247, 248, 250, chuA gene and fragments of the chuA gene which have conserved this nature B2/D+ A− such as SEQ ID NO: 241, 195, 185 or 248).

A subject of the present application is therefore any isolated polynucleotide which is of novel nature B2/D+ A− and which corresponds to a novel product in itself. It is in particular aimed towards any isolated polynucleotide the sequence of which corresponds to a sequence chosen from the group consisting of

the sequences SEQ ID NO: 1 to NO: 153,

the polynucleotide sequences which can be obtained by digesting the total DNA of an E. coli of group B2 or group D (such as, for example, an E. coli strain isolated from the blood of a patient suffering from a septicaemic or meningeal infection) with a restriction enzyme chosen from the group consisting of NotI and BlnI, selecting those of the fragments obtained which hybridize with at least one sequence chosen from the group consisting of SEQ ID NO: 134, 144, 109, 115, 140, 135, 33, 56, 122, 130, 141, 25, 48, 51, 57, 121, 44, 45, 113, 119, 120, 23, 52, and

the sequences of the orfs (open reading frames) which contain one of these sequences,

the nature-conserving variant or fractional sequences of these sequences.

The production of this polynucleotide group is described in detail in the examples which follow. It corresponds to the polynucleotides which are of novel nature B2/D+ A− and which are also novel as products. Besides the isolated fragments SEQ ID NO:1 to NO: 153, it comprises the novel regions 1, 3, 4 and 5 identified according to the invention, and the orfs which can be identified on these polynucleotides (cf. examples).

The expression “nature-conserving sequence of a sequence” is herein intended to mean any sequence which has conserved the nature B2/D+ A− (as defined above) of the polynucleotide corresponding to this parent sequence. The term “variant” is herein intended to mean any sequence having nucleotide insertions and/or deletions and/or substitutions with respect to the parent sequence, this includes any sequence which is complementary to the parent sequence. The term “fractional” is herein intended to mean any fragment of the parent sequence.

In the present application, any strain is considered to be E. coli if it can be considered to belong to the E. coli species according to the criteria given by Bergey's Manual of systemic bacteriology (cf. in particular Volume 1).

Similarly, any E. coli strain is considered to belong to the group B2 or to the group A if it is considered as such after carrying out a reference phylogenic test suitable for discriminating between B2/D and A, such as for example multilocus enzymatic electrophoresis (MLEE) and/or ribotyping. Such phylogenic techniques are well known to those skilled in the art. Examples of suitable protocols are in particular described in Herzer et al. 1990 J. Bacteriol. 172:6175-6181 and in Selander et al. 1986 Appl. Environ. Microbiol. 51:873-884 for MLEE, and in Bingen et al. 1994 Clin. Microbiol. Rev. 7:311-317, Bingen et al. Clin. Infect. Dis. 22:152-156, Bingen et al. J. Infect. Dis. 177:642-650 and in Desjardins et al. 1995 J. Mol. Evol. 41:440-448 for ribotyping.

Examples of such determinations are given in the examples which follow.

The fact of being or of not being present in a given bacterial strain or sample corresponds to a notion which is common to those skilled in the art in the field. In order to determine whether a given polynucleotide is, or is not, present in a given E. coli strain, the determination can in particular be carried out by Southern transfer of the nucleotide population of said E. coli strain, and bringing this transfer into contact with the polynucleotide tested, or with a probe derived from this polynucleotide, under conditions suitable for polynucleotide hybridization reactions. Examples of such probes and such conditions are given in Example 1. A positive hybridization will then be interpreted as the presence of said polynucleotide in said E. coli strain, and a hybridization which is negative or insignificant with respect to the background noise will then be interpreted as the absence of said polynucleotide in said E. coli strain. The determination can also be carried out by PCR detection of a positive or negative amplification using amplification primers constructed on the basis of said polynucleotide and placed in contact with the nucleotide population of said E. coli strain, under conditions which are favourable for the amplification, using these primers, of a target out of this population. Illustrations of such PCR procedures are given in the examples which follow.

The majority of the polynucleotides which are of nature B2/D+ A− according to the invention are advantageously present in ECOR E. coli of group B2 with a frequency greater than 10%, particularly greater than 40%, preferably greater than 50%, more preferably greater than 60%, and even more preferably greater than 70% (cf. Example 2 and Table 3). Some of them are present in ECOR E. coli of group B2 with a frequency greater than 80%, or even at a frequency equal to 100%, while at the same time being present in ECOR E. coli of group A at a frequency of less than 5%, or even less than 3%, or even equal to 0%.

The polynucleotides which are of nature B2/D+ A− according to the invention can be used for simply detecting the presence of E. coli bacteria of the group B2 or D with a probability of at least 90% (presence of said polynucleotides). Taken in combination together, or with other products, they make it possible to completely distinguish between the groups A, B1, B2 and D of E. coli (cf. Example 5). They also have therapeutic, palliative and/or preventive applications of interest. These polynucleotides will be referred to hereinafter as: novel polynucleotides which are of novel nature B2/D+ A−.

The present application is also aimed towards any pair of primers which allows the PCR amplification of at least one novel polynucleotide which is of novel nature B2/D+ A− as defined above. The setting up of suitable experimental PCR conditions is accessible to those skilled in the art (cf. in particular Molecular cloning—a laboratory manual, Sambrook, Fritsch, Maniatis, and in particular Vol. 2, Chap. 14 2nd edition; cf. Ausubel et al. 1989 Current protocols in molecular biology; John Wiley and Sons Ed., and in particular Vol. 2 Chap. 15). Examples thereof are given in the examples which follow.

It is in particular aimed towards the pair of primers corresponding to the pair SEQ ID NO: 164 and NO: 165. This specific pair of primers makes it possible to amplify the fragment SEQ ID NO: 119 (clone TspE4C2).

The present application is also aimed towards any nucleotide probe comprising at least one novel polynucleotide which is of novel nature B2/D+ A− as defined above. It is in particular aimed towards any probe as obtained using at least one pair of primers according to the invention, in particular by PCR amplification (cf. examples), such as (SEQ ID NO: 160; 161), (SEQ ID NO: 162; 163) or (SEQ ID NO: 164; 165).

In general, any pair of primers which allows the PCR amplification, under conditions as mentioned above, of at least one polynucleotide which is of novel nature B2/D+ A−, and also any probe directed against such a polynucleotide, and any antibody directed against a polypeptide encoded by such a polynucleotide, can, in accordance with the present invention, be used for the phylogenic determination of a bacterium of the E. coli species. The present invention also demonstrates that the yjaA gene (SEQ ID NO: 254; protein of SEQ ID NO: 255) is a phylogenic tool of interest for E. coli. The use, for the phylogenic determination of E. coli, of any product which makes it possible to detect yjaA (cf. Example 5) therefore enters into the domain of the present application. It is in particular aimed towards any pharmaceutical composition comprising such products, such as a pair of primers (e.g. SEQ ID NO: 162; NO: 163), a probe or a specific antibody.

The present application is also aimed towards any antisense polynucleotide, characterized in that its sequence corresponds to the antisense sequence of at least one novel polynucleotide which is of novel nature B2/D+A− and any isolated polypeptide, characterized in that its amino acid sequence corresponds to a sequence encoded, according to the universal genetic code and taking into account the degeneracy of this code, by at least one novel polynucleotide which is of novel nature B2/D+ A−.

The present application is also aimed towards any combination of the polypeptides encoded by the polynucleotides of the invention with a binding product, capable of binding to at least one novel polypeptide which is of novel nature B2/D+ A−, and which will inhibit an important biological function, such as the extra-intestinal growth by growth in serum, or the multiplication in animal, such as disclosed in example 6. Such products correspond in particular to antibodies or monoclonal antibodies and compounds inhibiting the biological function of the polypeptides. Methods for manufacturing such binding products using the polypeptides according to the invention are available to those skilled in the art. They are conventional methods which comprise, in particular, the immunization of animals such as rabbits and the harvesting of the serum produced, followed optionally by the purification of the serum obtained. A technique suitable for the production of monoclonal antibodies is that of Köhler and Milstein (Nature 1975, 256:495-497).

In the present invention, the expression “capable of binding” is intended to mean physiological-type conditions (in vivo or mimicking in vivo) when said binding product is intended to be administered to a human or animal organism, and ELISA-type conditions when said binding product is intended to be used in assays and methods in vitro, for example for determining the phylogenic group of an E. coli bacterium.

The present application is also aimed towards any vector comprising at least one novel polynucleotide which is of novel nature B2/D+ A−, and also any cell transformed by genetic engineering, characterized in that it comprises, by transfection, at least one novel polynucleotide which is of novel nature B2/D+ A− and/or at least one vector according to the invention, and/or in that said transformation induces the production by this cell of at least one polypeptide corresponding to a novel polynucleotide which is of novel nature B2/D+ A−.

A subject of the present application is also any composition, in particular any pharmaceutical composition, comprising at least one compound chosen from the group consisting of the novel polynucleotides which are of novel nature B2/D+ A−, the polypeptides corresponding to at least one novel polynucleotide which is of novel nature B2/D+ A−, and the vectors and cells according to the invention, mentioned above. Such compositions are in particular useful for treating and/or for alleviating and/or for preventing E. coli infections, and in particular of infection by extra-intestinal E. coli. (systemic and non-diarrhoeal infections). When said compound is immunogenic or is made immunogenic, these compositions can correspond to vaccines.

A subject of the present application is also any composition, in particular any pharmaceutical composition, comprising at least one compound chosen from the group consisting of the novel polynucleotides which are of novel nature B2/D+ A−, the pairs of primers and probes according to the invention, mentioned above, and the binding products according to the invention, mentioned above. Such a composition is in particular useful for the phylogenic determination of an E. coli bacterium. It can therefore correspond to a diagnostic kit or composition.

A subject of the present application is also any composition, in particular any pharmaceutical composition, comprising, in association with an inert carrier, at least one compound chosen from the group consisting of the antisense polynucleotides according to the invention, mentioned above, and the binding products according to the invention, mentioned above. Such compositions can also be used for the phylogenic determination of an E. coli bacterium, and can take the form of a diagnostic kit or composition. They can also be used in a therapeutic capacity, in order to treat and/or to alleviate and/or to prevent any undesirable growth of E. coli, such as a sanitary contamination, an E. coli infection, and in particular the presence of extra-intestinal E. coli (cf. examples).

A subject of the present application is also any use of these products according to the invention for manufacturing such compositions.

The present invention also provides products for which the sequence exhibits homologies with known products, but for which the nature B2/D+ A− is novel. There are in particular the sequences SEQ ID NO: 170, 171, 174, 175, 178, 179, 183, 185, 186, 190, 191, 193, 194, 195, 196, 199, 200, 202, 205, 206, 208, 209, 211, 214, 218, 220, 221, 233, 234, 235, 241, 244, 246, 247, 248, 250, the orfs (open reading frames) containing these sequences, the chuA gene, the operon of this gene and of its nature-conserving fragments and variants, and the polynucleotides corresponding to a region 2, 6a and 6b of E. coli as described in Example 1 which follows, with the exception of the polynucleotides sfa, hly, cnf1, pap/prs, hra and ibe 10. These products will be referred to hereinafter as: products which exhibit homology but which are of novel nature B2/D+ A−.

The chuA gene is a known product (Genbank Accession No. U67920): it has been described in a few E. coli responsible for intestinal and extra-intestinal infections, in Shigella as a gene involved in iron metabolism, and has been described as being homologous to a Yersinia gene. However, it has never been described as a phylogenic marker. Now, the present invention reports, for the first time, its presence in the urophathogenic E. coli strain J96 and reports, for the first time, that it is present in 100% of the B2s and of the Ds and in 0% of the As and of the B1s of the ECOR collection. Four fragments of chuA which are of nature B2/D+ A− are also described (SEQ ID NO: 241, 195, 185 and 248).

The majority of the polynucleotides according to the invention which exhibit homology but which are of novel nature B2/D+ A− are advantageously present in ECOR E. coli of group B2 with a frequency greater than 40%, preferably greater than 50%, more preferably greater than 60% and even more preferably greater than 70%. Some of them are present in ECOR E. coli of group B2 with a frequency greater than 80%, or even at a frequency equal to 100%, while at the same time remaining present in ECOR E. coli of group A at a frequency of less than 5%, or even less than 3%, or even equal to 0% (cf. Example 2).

The present application is thus aimed towards any use of a compound which is of novel nature B2/D+ A− for the phylogenic determination of an E. coli bacterium, for the diagnosis of the presence or absence of undesirable E. coli bacteria, such as contaminant E. coli or extra-intestinal E. coli, and/or for the diagnosis of an E. coli infection, and/or for the manufacture of a composition intended for such a phylogenic determination and/or for such a diagnosis.

It is in particular aimed towards any use of at least one compound chosen from the group consisting of:

the polynucleotides corresponding to the chuA gene and to its operon,

the polynucleotides the sequence of which corresponds to SEQ ID NO: 170, 171, 174, 175, 178, 179, 183, 185, 186, 190, 191, 193, 194, 195, 196, 199, 200, 202, 205, 206, 208, 209, 211, 214, 218, 220, 221, 233, 234, 235, 241, 244, 246, 247, 248 and 250,

the polynucleotides which can be obtained by digesting the total DNA of an E. coli of group B2 or D with a restriction enzyme chosen from the group consisting of NotI and BlnI, and selecting those of the fragments obtained which hybridize to at least one sequence chosen from the group consisting of SEQ ID NO: 125, 123, 116, 43, 40, 127, 133, 27, 34, 36, 42, 46, 54, 55, 38, 128 and 151, with the exception of the polynucleotides sfa, hly, cnf1, pap/prs, hra and ibe 10,

the polynucleotides the sequence of which corresponds to an orf comprising the sequence of one of the polynucleotides cited in the preceding three paragraphs,

the polynucleotides the sequence of which corresponds to a nature-conserving variant or fractional sequence of the sequence of at least one of these polynucleotides (and in particular SEQ ID NO: 241, 195, 185 and 248),

the pair of primers, the probes and the antisense polynucleotides, the sequence of which is as derived from these polynucleotides,

the binding products which are capable of binding to a polypeptide encoded by a polynucleotide which is of novel nature B2/D+ A−, for the phylogenic determination of an E. coli bacterium.

The present invention is also aimed towards the use of at least one of these compounds for the diagnosis of the presence or absence of undesirable E. coli bacteria, such as contaminant E. coli or extra-intestinal E. coli, and/or for the diagnosis of an E. coli infection. It is also aimed towards any use of at least one of these compounds for the manufacture of a composition intended for such a phylogenic determination and/or for such a diagnosis.

These compounds chuA, SEQ ID NO: 170, 171, 174, 175, 178, 179, 183, 185, 186, 190, 191, 193, 194, 195, 196, 199, 200, 202, 205, 206, 208, 209, 211, 214, 218, 220, 221, 233, 234, 235, 241, 244, 246, 247, 248, 250, and the nature-conserving variant or fractional polynucleotides of these products correspond, in fact, to products which are of novel nature B2/D+ A− (as defined above). The detection of the presence or absence of such compounds can in particular be carried out by nucleotide hybridization, by PCR amplification or by detection of their polypeptide products. Detection of the presence of such compounds makes it possible to conclude that the B2 or D E. coli bacterium is present. The combined use of these compounds, or the use of one or more of these compounds combined with other products, such as in particular yjaA, makes it possible to refine the phylogenic allocation. The combined use of the detection of the presence or absence of chuA and of yjaA (SEQ ID NO: 254) makes it possible in particular to conclude as to whether E. coli of group B2 and/or E. coli of group D are present or absent (cf. Example 5).

The present application is also aimed towards any use of a compound which is of novel nature B2/D+ A− for the manufacture of a composition, in particular of a pharmaceutical composition, intended to alleviate and/or to prevent and/or to treat an undesirable growth of E. coli, such as an E. coli infection, the presence of extra-intestinal E. coli or a sanitary contamination. It is aimed in particular towards any use of at least one compound chosen from the group consisting of:

the polynucleotides corresponding to the chuA gene and to its operon,

the polynucleotides the sequence of which corresponds to SEQ ID NO: 170, 171, 174, 175, 178, 179, 183, 185, 186, 190, 191, 193, 194, 195, 196, 199, 200, 202, 205, 206, 208, 209, 211, 214, 218, 220, 221, 233, 234, 235, 241, 244, 246, 247, 248 and 250,

the polynucleotides which can be obtained by digesting the total DNA of an E. coli of group B2 or D with a restriction enzyme chosen from the group consisting of NotI and BlnI, and selecting those of the fragments obtained which hybridize to at least one sequence chosen from the group consisting of SEQ ID NO: 125, 123, 116, 43, 40, 127, 133, 27, 34, 36, 42, 46, 54, 55, 38, 128 and 151, with the exception of the polynucleotides sfa, hly, cnf1, pap/prs, hra and ibe 10,

the polynucleotides the sequence of which corresponds to an orf (open reading frame) comprising the sequence of one of the polynucleotides cited in the preceding three paragraphs,

the polynucleotides the sequence of which corresponds to a nature-conserving variant or fractional sequence of the sequence of at least one of these polynucleotides (and in particular SEQ ID NO: 241, 195, 185 and 248),

the polynucleotides which are antisense polynucleotides of the polynucleotides of this group,

the polypeptides the sequence of which corresponds, according to the universal genetic code and taking into account the degeneracy of this code, to these polynucleotides,

the binding products (for example, antibodies) capable of binding to a polypeptide encoded by a polynucleotide which is of novel nature B2/D+A−,

the vectors comprising at least one of these polynucleotides,

the cells transformed by genetic engineering which comprise at least one of these polynucleotides and/or at least one of these vectors, and/or the transformation of which induces the production of at least one of these polypeptides, for the manufacture of a composition, in particular of a pharmaceutical composition, intended to alleviate and/or to prevent and/or to treat an undesirable growth of E. coli, such as an E. coli infection, the presence of extra-intestinal E. coli or a sanitary contamination.

According to another applied aspect, the present application is aimed towards any method which makes it possible to identify a compound capable of inhibiting the growth of E. coli, and in particular of inhibiting its extra-intestinal development, which comprises the detection of at least one compound:

i. capable of binding to at least one polynucleotide chosen from the group consisting of:

the novel polynucleotides which are of novel nature B2/D+ A−, i.e.

the sequences SEQ ID NO: 1 to NO: 153,

the polynucleotide sequences which can be obtained by digesting the total DNA of an E. coli of group B2 or group D (such as, for example, an E. coli strain isolated from the blood of a patient suffering from a septicaemic or meningeal infection) with a restriction enzyme chosen from the group consisting of NotI and BlnI, selecting those of the fragments obtained which hybridize with at least one sequence chosen from the group consisting of SEQ ID NO: 134, 144, 109, 115, 140, 135, 33, 56, 122, 130, 141, 25, 48, 51, 57, 121, 44, 45, 113, 119, 120, 23, 52 and sequencing selected fragments, and

the sequences of the orfs (open reading frames) which contain one of these sequences,

the nature-conserving variant or fractional sequences of these sequences,

the polynucleotides corresponding to the chuA gene and to its operon,

the polynucleotides the sequence of which corresponds to SEQ ID NO: 170, 171, 174, 175, 178, 179, 183, 185, 186, 190, 191, 193, 194, 195, 196, 199, 200, 202, 205, 206, 208, 209, 211, 214, 218, 220, 221, 233, 234, 235, 241, 244, 246, 247, 248 and 250,

the polynucleotides which are as obtained by digesting the total DNA of an E. coli of group B2 or D with a restriction enzyme chosen from the group consisting of NotI and BlnI, and selecting those of the fragments obtained which hybridize to at least one sequence chosen from the group consisting of SEQ ID NO: 125, 123, 116, 43, 40, 127, 133, 27, 34, 36, 42, 46, 54, 55, 38, 128 and 151, with the exception of the polynucleotides sfa, hly, cnf1, pap/prs, hra and ibe 10,

the polynucleotides the sequence of which corresponds to an orf comprising the sequence of one of the polynucleotides cited in the preceding four paragraphs,

the polynucleotides the sequence of which corresponds to a nature-conserving variant or fractional sequence of the sequence of at least one of these polynucleotides and in particular (SEQ ID NO: 241, 195, 185 and 248), and

ii. capable of specifically inhibiting the correct transcription and/or translation of this polynucleotide.

The present invention is also aimed towards any method which makes it possible to identify a compound capable of inhibiting the growth of an E. coli bacterium, and in particular its extra-intestinal development, characterized in that it comprises the detection of at least one compound capable of inhibiting the activity of a protein encoded by a polynucleotide the orf of which comprises a polynucleotide chosen from the group consisting of

the novel polynucleotides which are of novel nature B2/D+ A− according to the invention, i.e.

the sequences SEQ ID NO: 1 to NO: 153,

the polynucleotide sequences which can be obtained by digesting the total DNA of an E. coli of group B2 or group D (such as, for example, an E. coli strain isolated from the blood of a patient suffering from a septicaemic or meningeal infection) with a restriction enzyme chosen from the group consisting of NotI and BlnI, selecting those of the fragments obtained which hybridize with at least one sequence chosen from the group consisting of SEQ ID NO: 134, 144, 109, 115, 140, 135, 33, 56, 122, 130, 141, 25, 48, 51, 57, 121, 44, 45, 113, 119, 120, 23, 52 and sequencing selected fragments, and

the sequences of the orfs (open reading frames) which contain one of these sequences,

the nature-conserving variant or fractional sequences of these sequences.

the polynucleotides corresponding to the chuA gene and to its operon,

the polynucleotides the sequence of which corresponds to SEQ ID NO: 170, 171, 174, 175, 178, 179, 183, 185, 186, 190, 191, 193, 194, 195, 196, 199, 200, 202, 205, 206, 208, 209, 211, 214, 218, 220, 221, 233, 234, 235, 241, 244, 246, 247, 248 and 250,

the polynucleotides which are as obtained by digesting the total DNA of an E. coli of group B2 or D with a restriction enzyme chosen from the group consisting of NotI and BlnI, and selecting those of the fragments obtained which hybridize to at least one sequence chosen from the group consisting of SEQ ID NO: 125, 123, 116, 43, 40, 127, 133, 27, 34, 36, 42, 46, 54, 55, 38, 128 and 151, with the exception of the polynucleotides sfa, hly, cnf1, pap/prs, hra and ibe 10,

the polynucleotides the sequence of which corresponds to an orf (open reading frame) comprising the sequence of one of these polynucleotides,

the polynucleotides the sequence of which corresponds to a nature-conserving variant or fractional sequence of the sequence of at least one of these polynucleotides.

Such compounds can in particular be obtained by screening chemical and/or biological libraries.

The present application is also directed towards any compound as identified by one or other of these methods, and any composition, in particular any pharmaceutical composition, comprising at least one such compound. Such compositions are in particular useful for treating and/or for alleviating and/or for preventing an undesirable growth of E. coli, such as E. coli infections, or an E. coli contamination, and especially useful for treating and/or for alleviating and/or for preventing the presence of extra-intestinal E. coli bacteria.

A subject of the present invention is also, according to a notable aspect of the invention, phylogenic identification methods which implement the detection of the presence or absence of at least one of the polynucleotides which are of novel nature B2/D+ A− (whether these polynucleotides are novel as products, or whether they exhibit homologies with known products):

the novel polynucleotides according to the invention, i.e.

the sequences SEQ ID NO: 1 to NO: 153,

the polynucleotide sequences which can be obtained by digesting the total DNA of an E. coli of group B2 or group D (such as, for example, an E. coli strain isolated from the blood of a patient suffering from a septicaemic or meningeal infection) with a restriction enzyme chosen from the group consisting of NotI and BlnI, selecting those of the fragments obtained which hybridize with at least one sequence chosen from the group consisting of SEQ ID NO: 134, 144, 109, 115, 140, 135, 33, 56, 122, 130, 141, 25, 48, 51, 57, 121, 44, 45, 113, 119, 120, 23, 52 and sequencing selected fragments, and

the sequences of the orfs (open reading frames) which contain one of these sequences,

the nature-conserving variant or fractional sequences of these sequences,

the polynucleotides corresponding to the chuA gene and to its operon,

the polynucleotides the sequence of which corresponds to SEQ ID NO: 170, 171, 174, 175, 178, 179, 183, 185, 186, 190, 191, 193, 194, 195, 196, 199, 200, 202, 205, 206, 208, 209, 211, 214, 218, 220, 221, 233, 234, 235, 241, 244, 246, 247, 248 and 250,

the polynucleotides which can be obtained by digesting the total DNA of an E. coli of group B2 or D with a restriction enzyme chosen from the group consisting of NotI and BlnI, and selecting those of the fragments obtained which hybridize to at least one sequence chosen from the group consisting of SEQ ID NO: 125, 123, 116, 43, 40, 127, 133, 27, 34, 36, 42, 46, 54, 55, 38, 128 and 151, with the exception of the polynucleotides sfa, hly, cnf1, pap/prs, hra and ibe 10,

the polynucleotides the sequence of which corresponds to an orf (open reading frame) comprising the sequence of one of these polynucleotides,

the polynucleotides the sequence of which corresponds to a nature-conserving variant or fractional sequence of the sequence of at least one of these polynucleotides.

This detection can be carried out by direct detection of said polynucleotide, or of its fragments, or by detection of one or more polypeptide(s) corresponding to it (polypeptides encoded by a polynucleotide which is of novel nature B2/D+ A−). In particular, the present application is aimed towards any phylogenic identification method characterized in that it comprises the use of at least one compound chosen from the group consisting of:

the novel polynucleotides which are of novel nature B2/D+ A−, i.e.

the sequences SEQ ID NO: 1 to NO: 153,

the polynucleotide sequences which can be obtained by digesting the total DNA of an E. coli of group B2 or group D (such as, for example, an E. coli strain isolated from the blood of a patient suffering from a septicaemic or meningeal infection) with a restriction enzyme chosen from the group consisting of NotI and BlnI, selecting those of the fragments obtained which hybridize with at least one sequence chosen from the group consisting of SEQ ID NO: 134, 144, 109, 115, 140, 135, 33, 56, 122, 130, 141, 25, 48, 51, 57, 121, 44, 45, 113, 119, 120, 23, 52 and sequencing selected fragments, and

the sequences of the orfs (open reading frames) which contain one of these sequences,

the nature-conserving variant or fractional sequences of these sequences,

the polynucleotides corresponding to the chuA gene and to its operon,

the polynucleotides the sequence of which corresponds to SEQ ID NO: 170, 171, 174, 175, 178, 179, 183, 185, 186, 190, 191, 193, 194, 195, 196, 199, 200, 202, 205, 206, 208, 209, 211, 214, 218, 220, 221, 233, 234, 235, 241, 244, 246, 247, 248 and 250,

the polynucleotides which are as obtained by digesting the total DNA of an E. coli of group B2 or D with a restriction enzyme chosen from the group consisting of NotI and BlnI, and selecting those of the fragments obtained which hybridize to at least one sequence chosen from the group consisting of SEQ ID NO: 125, 123, 116, 43, 40, 127, 133, 27, 34, 36, 42, 46, 54, 55, 38, 128, 151 and 211, with the exception of the polynucleotides sfa, hly, cnf1, pap/prs, hra and ibe 10,

the polynucleotides the sequence of which corresponds to a nature-conserving variant or fractional sequence of the sequence of a polynucleotide of this group (and in particular SEQ ID NO: 241, 195, 185 and 248),

the polynucleotides the sequence of which corresponds to an orf comprising the sequence of a polynucleotide of this group,

the pairs of primers which make it possible to amplify a polynucleotide which is of novel nature B2/D+ A−, as defined above,

the probes comprising a polynucleotide which is of novel nature B2/D+ A−, as defined above,

the polynucleotides which are antisense polynucleotides of a polynucleotide which is of novel nature B2/D+ A−, as defined above,

the compounds as identified by a method according to the invention for identifying compounds capable of inhibiting the growth of E. coli,

the binding products which are capable of binding to a polynucleotide which is of novel nature B2/D+ A−, as defined above,

the binding products (for example, antibodies) capable of binding to a polypeptide encoded by a polynucleotide which is of novel nature B2/D+ A− as defined above.

This implementation can be carried out according to any technique known to those skilled in the art so as to allow said “at least one compound” to detect, in a biological sample, the presence or absence of the polynucleotide(s) or, where appropriate, the polypeptide(s) which constitute(s) the target thereof: ELISA for reactions of antibody-antigen type, Southern-type hybridization or PCR for reactions of polynucleotide hybridization and/or amplification type.

Suitable biological samples comprise, in particular, all samples of human origin originating from sites which are normally sterile (blood, cerebrospinal fluid -CSF-, liquid from an effusion, etc.) or non-sterile (stools, oropharynx, skin, etc.), and also samples of environmental origin (soils, water, etc.), of food origin and of animal origin, and microbiological cultures.

This method makes it possible to conclude, when said detection is positive, that E. coli bacteria of group B2 and/or D are present. With only the detection of the presence or absence of chuA, this method makes it possible to conclude, when this detection is positive, that E. coli bacteria of group B2 or D are present. When this detection is negative, and insofar as the sample effectively contains E. coli, it makes it possible to conclude that bacteria of group B1 or A are present.

This method can also comprise the use of several of said compounds, for example in such a way as to detect the presence or absence of the chuA gene, and also that of the fragment SEQ ID NO: 119. It makes it possible then to conclude:

when this detection is negative for chuA and positive for SEQ ID NO: 119, that E. coli bacteria of group B1 are present,

when this detection is negative for chuA and negative for SEQ ID NO: 119, that E. coli bacteria of group A are present.

Examples of such methods and detection are given in the examples which follow. A pair of primers SEQ ID NO: 160 and NO: 161 which makes it possible to detect the presence or absence of the chuA gene, and a pair of primers SEQ ID NO: 164 and 165 which makes it possible to detect the presence or absence of SEQ ID NO: 119 (TspE4.C2) are described in particular.

The phylogenic identification method according to the invention can also implement the detection of the presence or absence of the yjaA gene. The yjaA gene (cf. FIG. 5) is, in the prior art, known to be present in the E. coli strain K12 (group A). There is nothing in the prior art which would suggest that it may constitute a phylogenic marker capable of distinguishing between group B2 and group D.

FIG. 5 shows the yjaA sequence (SEQ ID NO: 254) and that of the corresponding ORF (SEQ ID NO: 255).

The detection of the presence or absence of the yjaA gene can take place by any technique accessible to those skilled in the art. It can in particular take place by detection of the polynucleotide or of its fragments (SEQ ID NO: 254), or by detection of the polypeptides which correspond to it (SEQ ID NO: 255). The development of reagents which allow such a detection is accessible to those skilled in the art: amplification probes or primers for the detection of polynucleotides, compound of serum or antibody type for the detection of polypeptides. The polynucleotide hybridization reactions can, for example, be carried out by Southern and/or by PCR, and those of antigen-antibody type can be carried out by ELISA. Examples of implementation of such a detection step are given in the examples which follow. A pair of primers (SEQ ID NO: 162 and 163) which makes it possible to amplify yjaA are described in particular.

The present invention is also aimed towards any phylogenic identification method which implements at least the detection of the presence or absence of the yjaA gene. This detection makes it possible to distinguish between E. coli of group B2 and E. coli of group D (cf. examples).

Notably, the present invention provides a phylogenic detection method which makes it possible to completely distinguish the E. coli groups A, B1, B2 and D. This method implements the detection of the presence or absence of the chuA gene, of the yjaA gene and of SEQ ID NO: 119. This method is described in detail in the examples. Advantageous PCR conditions are in particular are mentioned therein (“simplified PCR”).

Advantageously, this detection can be carried out by PCR amplification. Examples of suitable PCR conditions are given in a purely illustrative capacity in the examples.

The present application is in particular aimed towards an identification method as defined above which implements the simultaneous detection of one or more of said polynucleotides which is/are of nature B2/D+ A−, and of yjaA, preferably by triplex PCR. Particularly advantageously, this method can be applied directly to the bacteria or to the sample analysed. It does not require the availability of a reference strain collection.

The present application is also aimed towards any method for detecting an undesirable growth of E. coli, any method for diagnosing an E. coli infection, any method for sanitary control or detection (foodstuffs, liquids intended for consumption, soils) and any method for selecting E. coli strains suitable for biotechnological manipulations, which implement the phylogenic identification method according to the invention.

It is also aimed towards any kit for implementing a phylogenic identification, diagnostic and/or selection method according to the invention, possibly accompanied by instructions for use. Such kits can in particular comprise at least one of the compounds used in said method. They can also comprise instructions for use featuring one or more of the profiles given in FIG. 4 (cf. Example 5), or describing one or more of these profiles.

An advantageous kit comprises at least one of the pairs of primers (SEQ ID NO: 160, 161), (SEQ ID NO: 162, 163) and (SEQ ID NO: 164, 165), preferably two of these pairs, more preferably the three pairs of primers.

The present invention is illustrated by the examples which follow and which are given in a nonlimiting capacity.

Other advantages and variants of implementation can be read by those skilled in the art in these examples. Such variants are targeted by the present application.

In these examples, reference is made to the following figures:

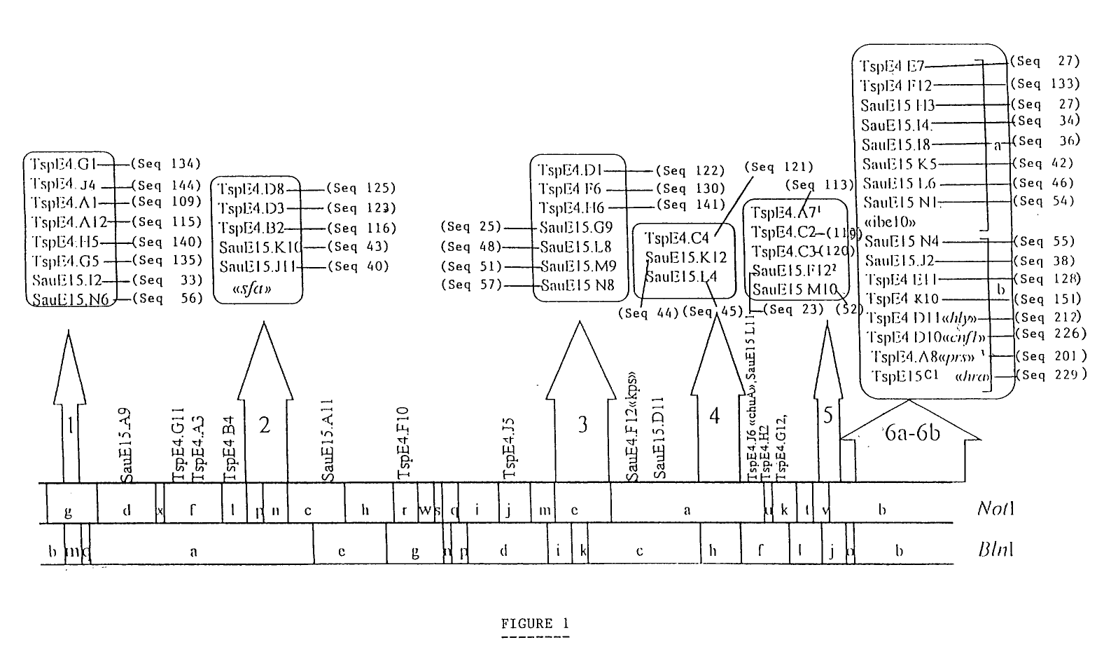

FIG. 1 represents the distribution of B2/D+ A− clones along the chromosome of the E. coli strain RS218,

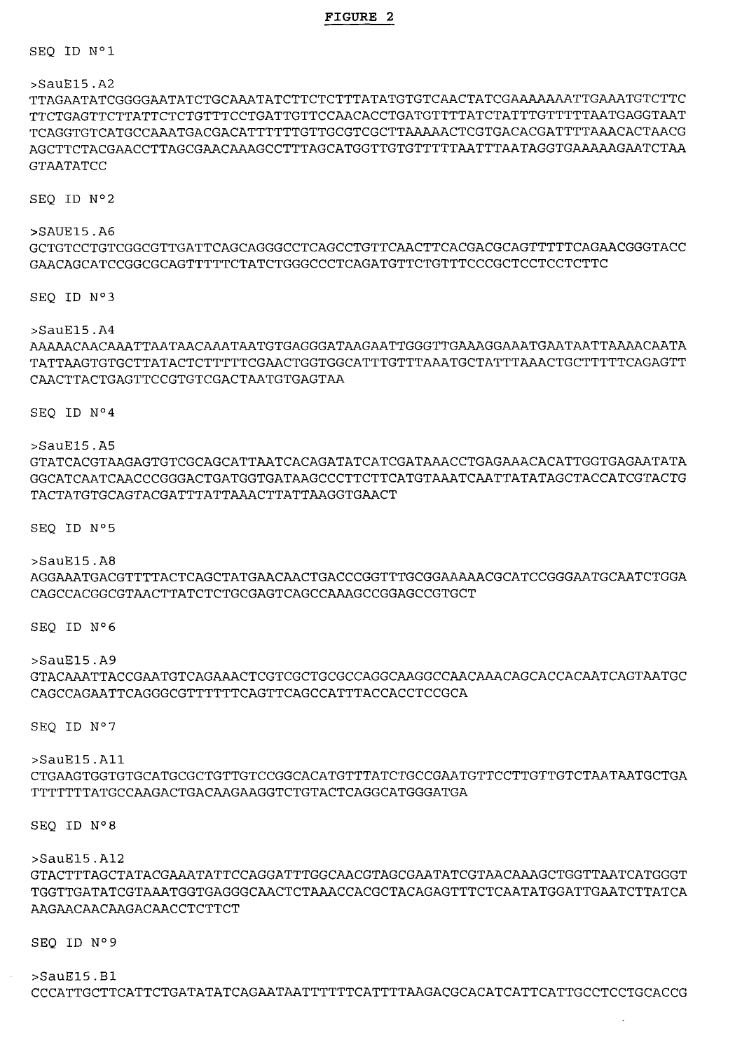

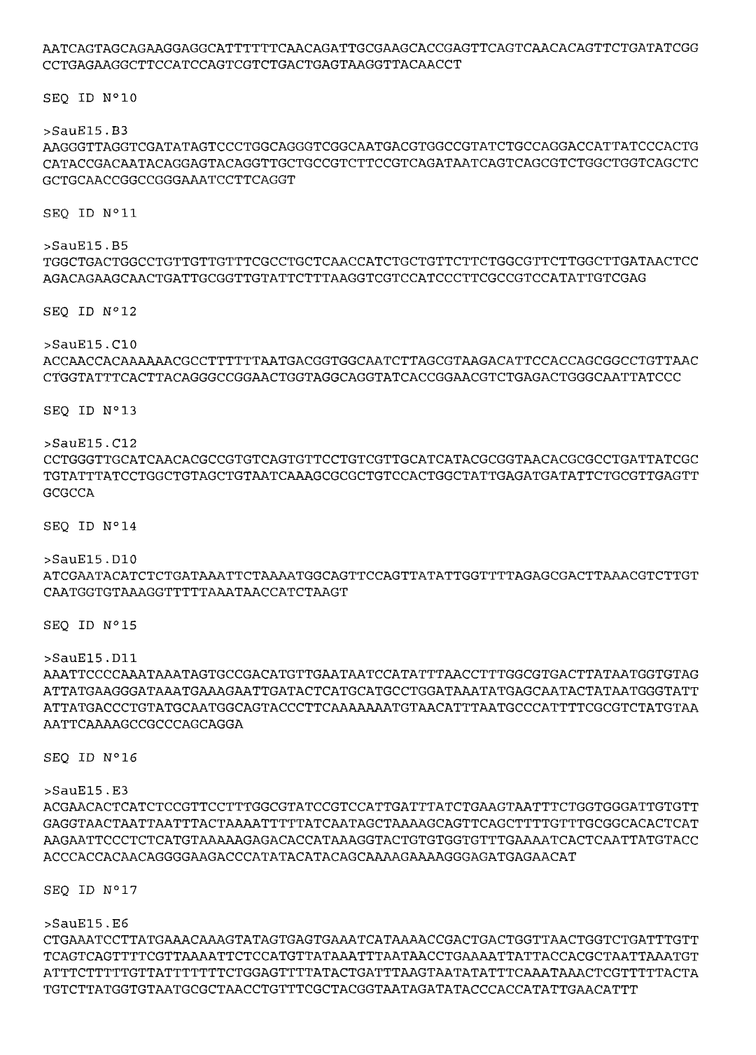

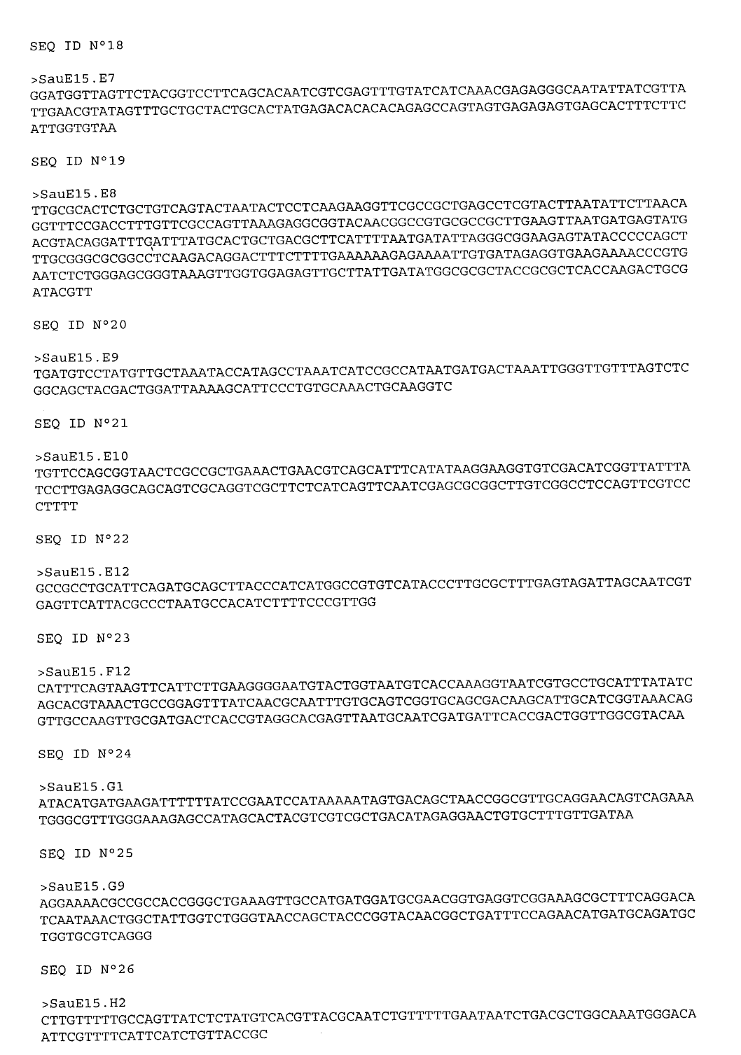

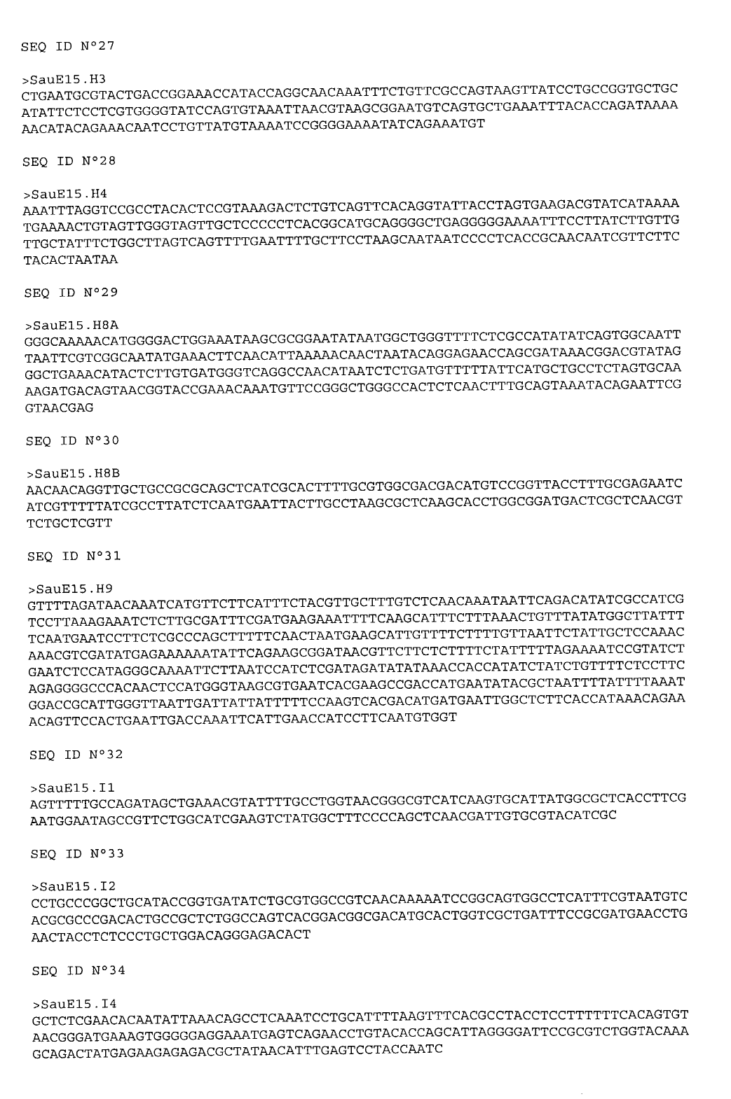

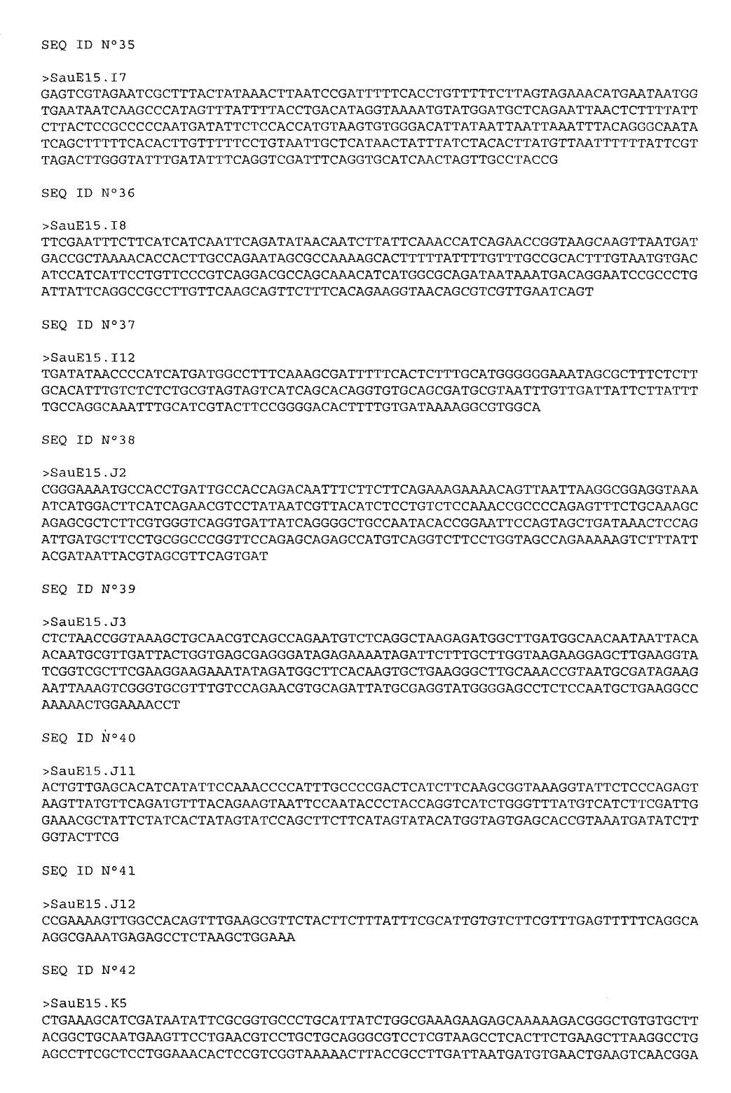

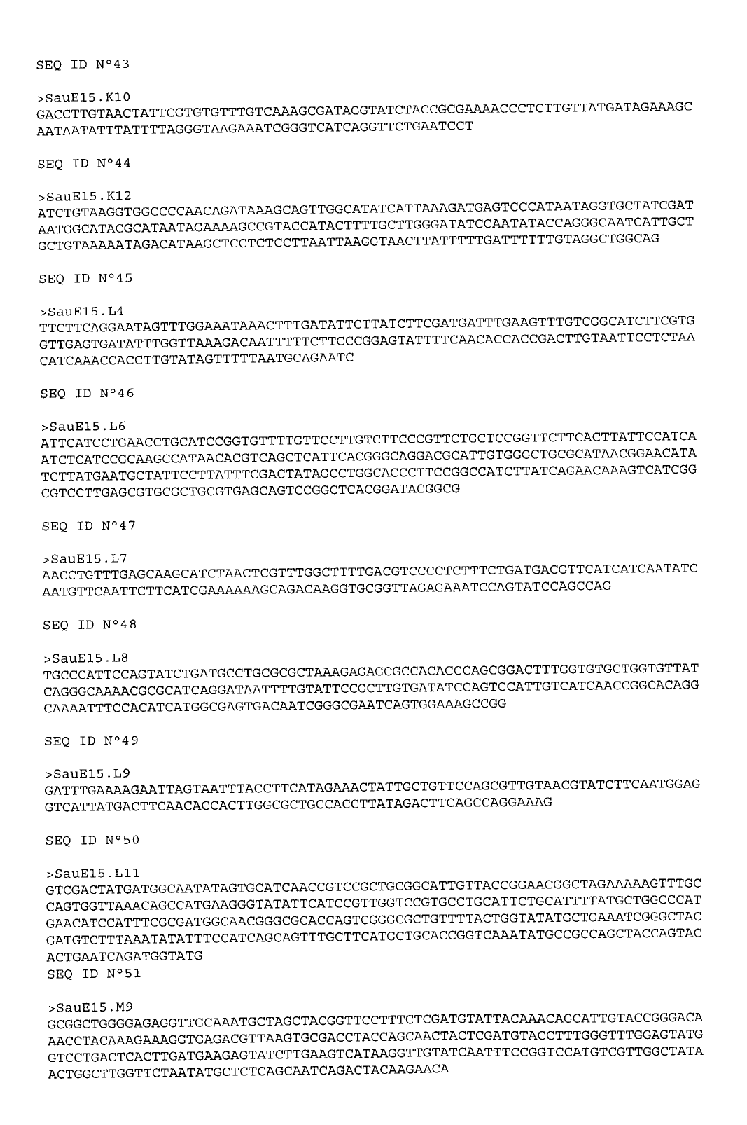

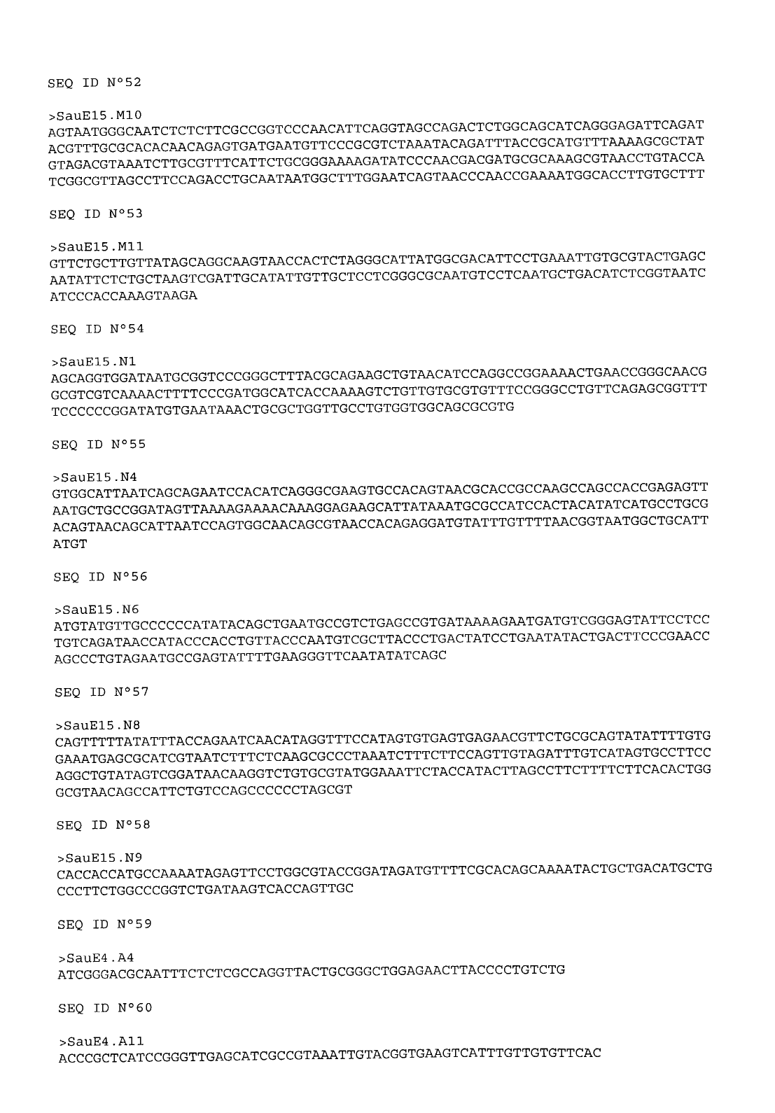

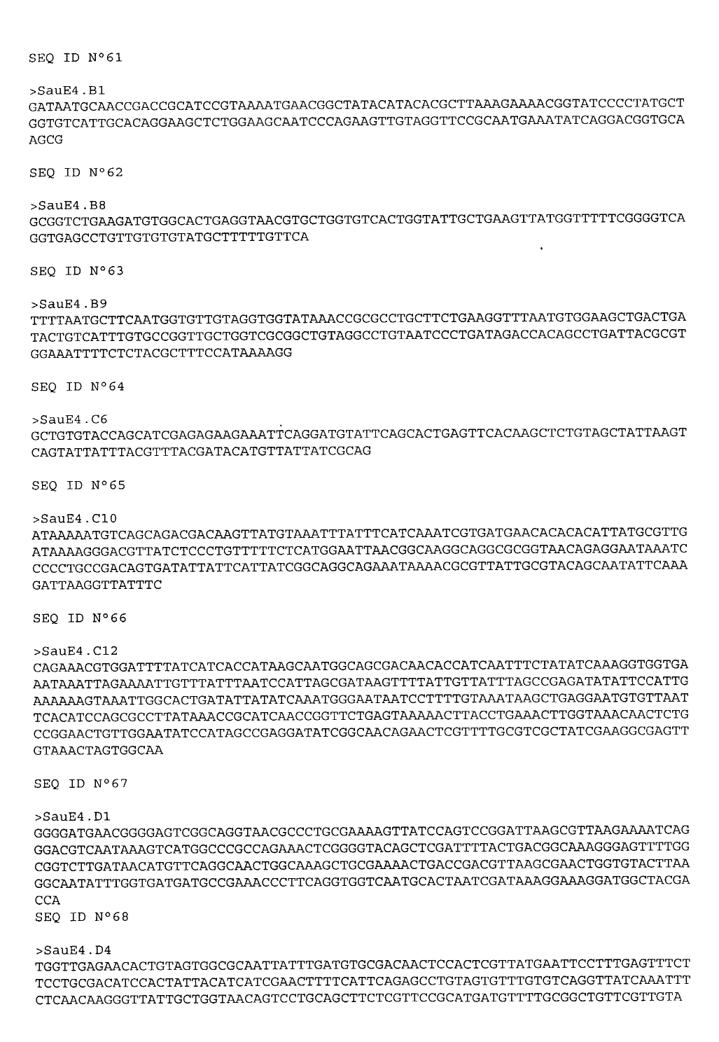

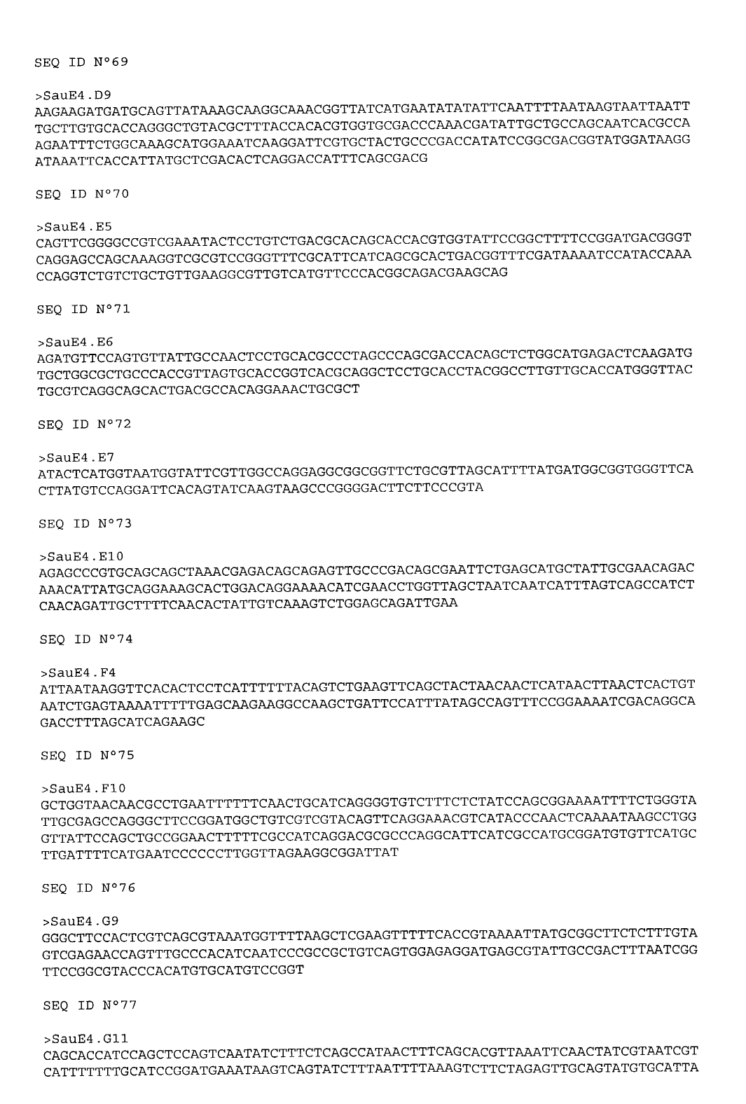

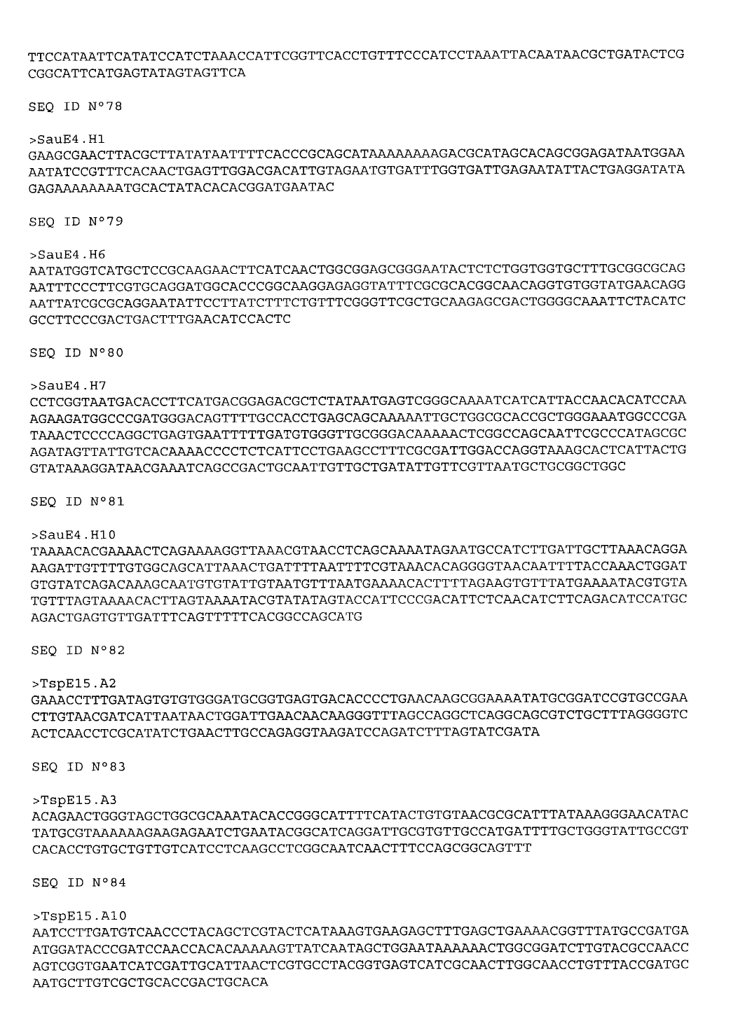

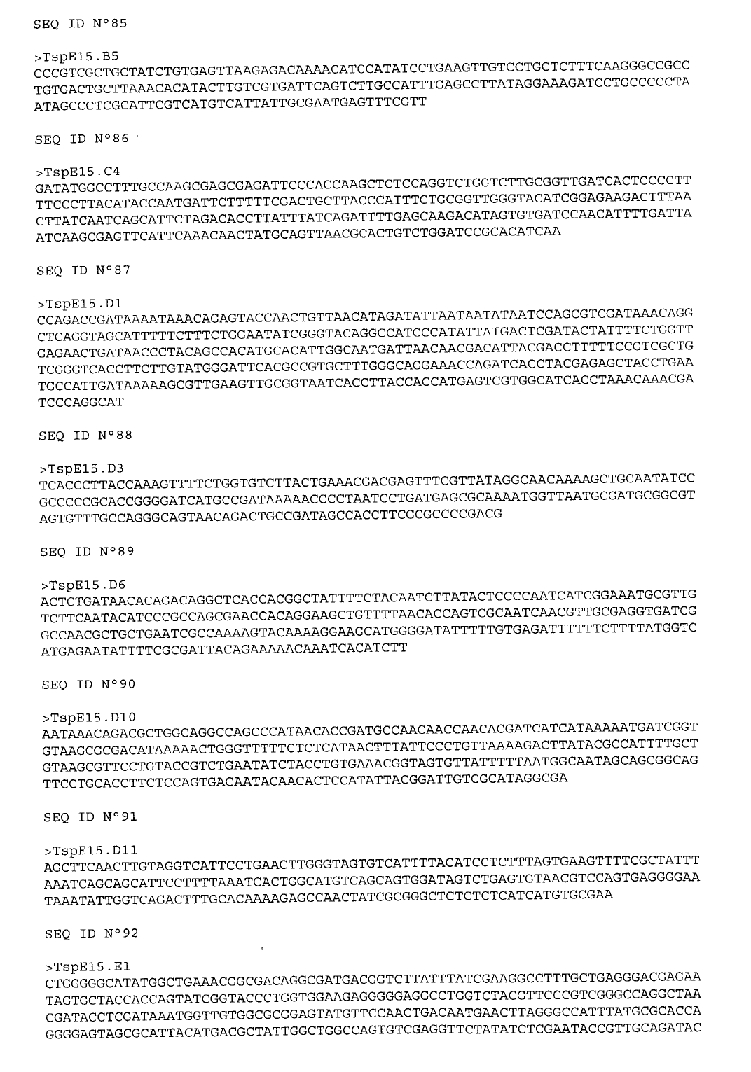

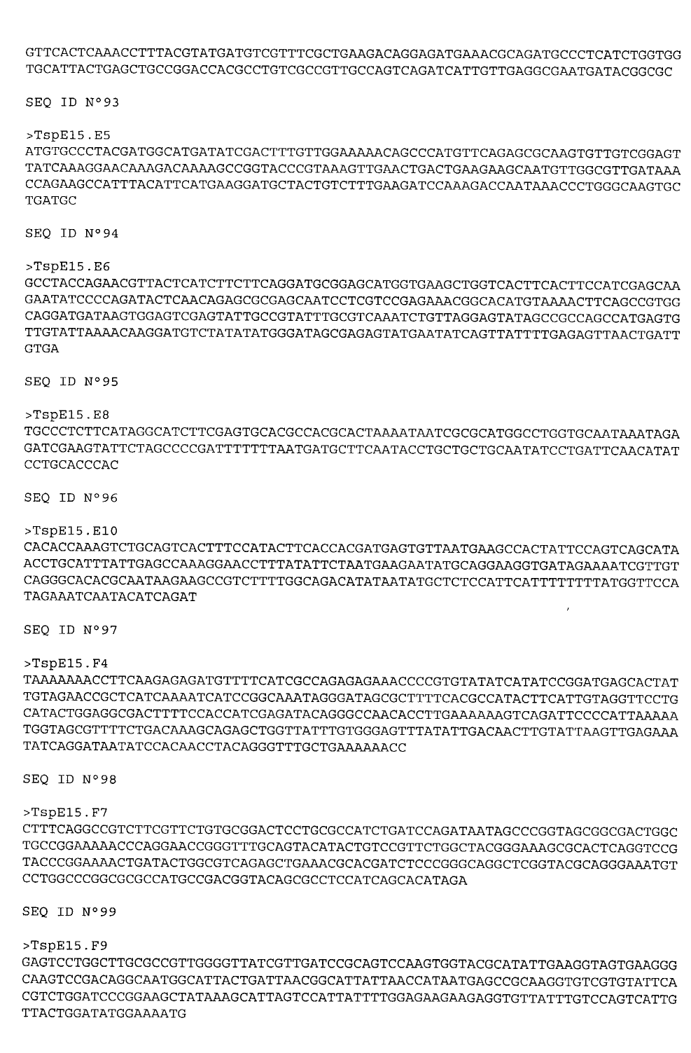

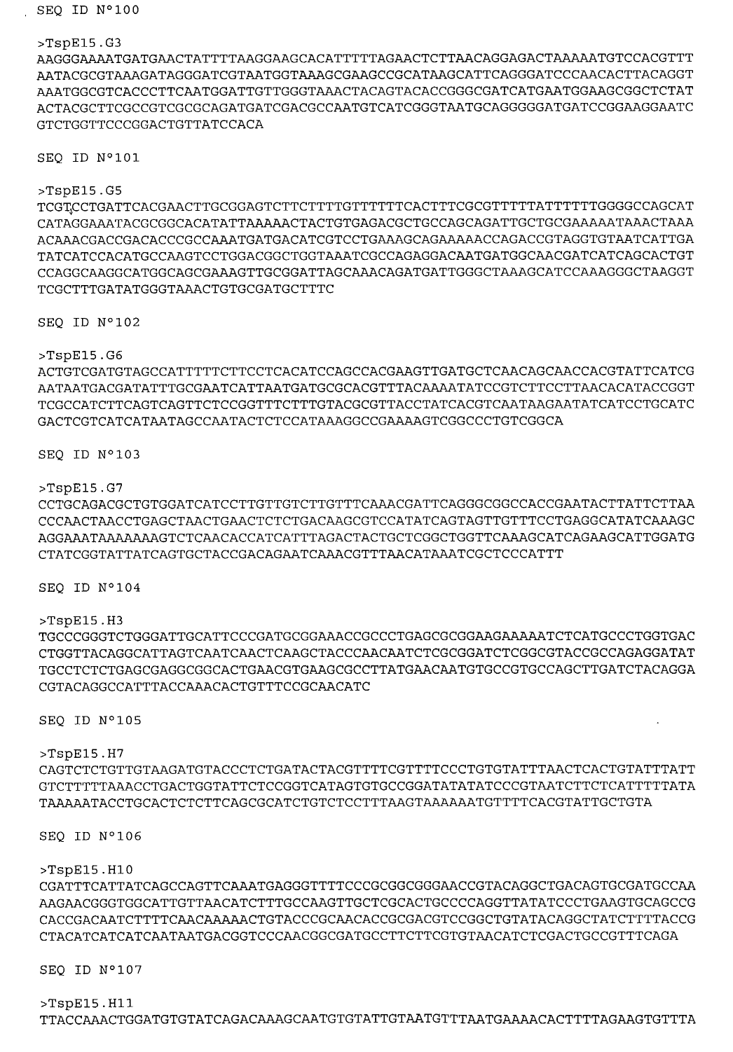

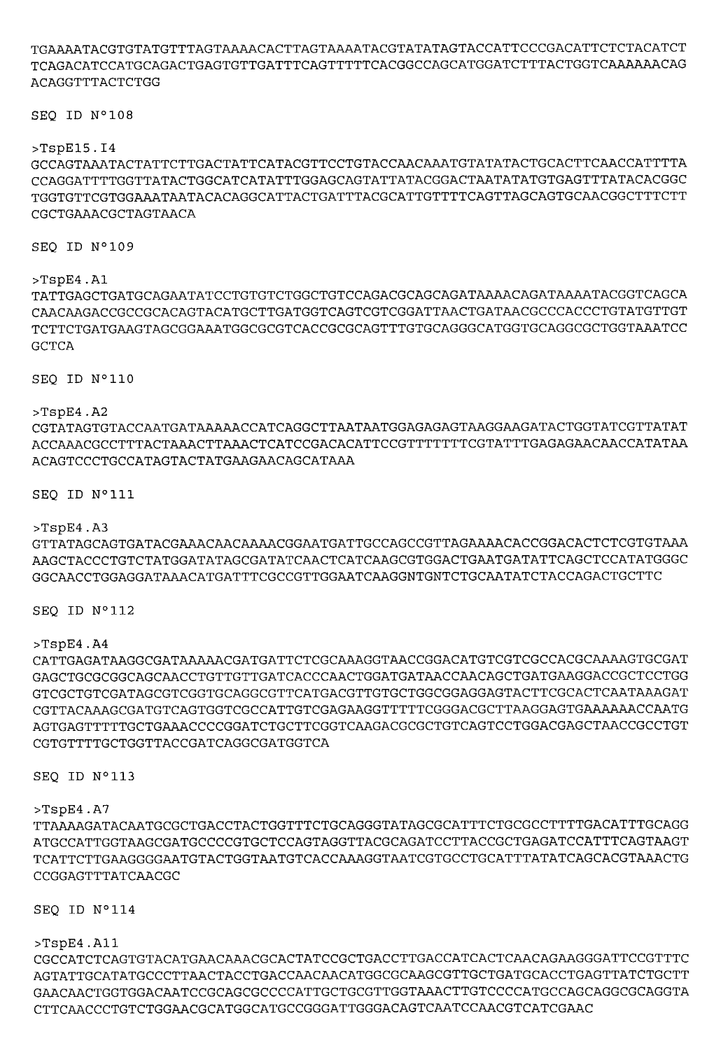

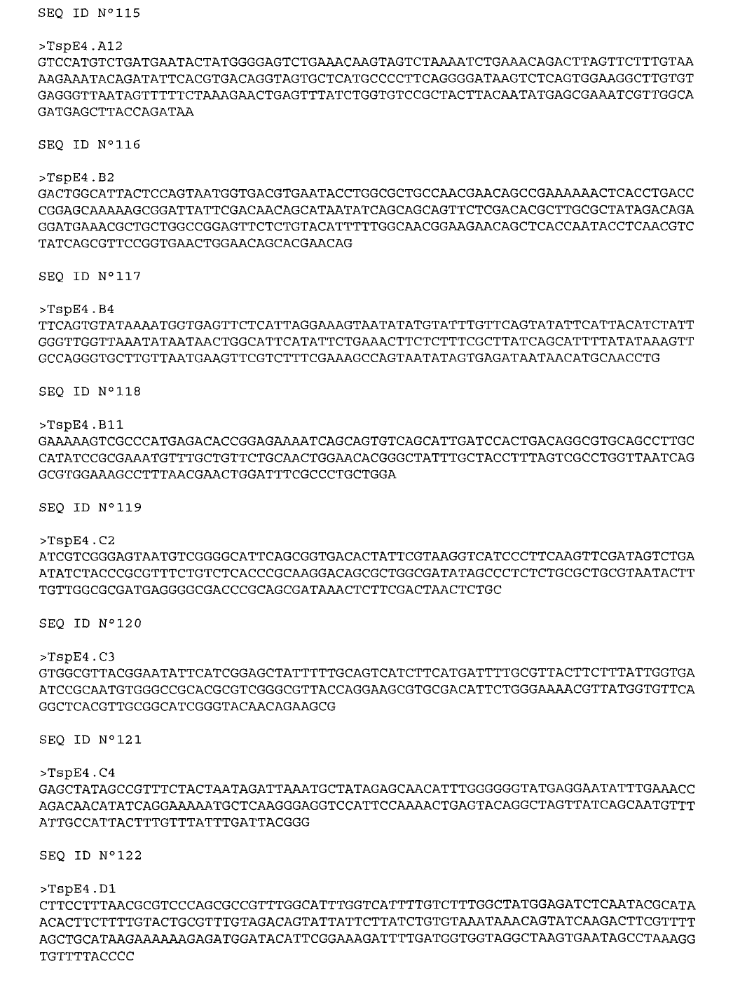

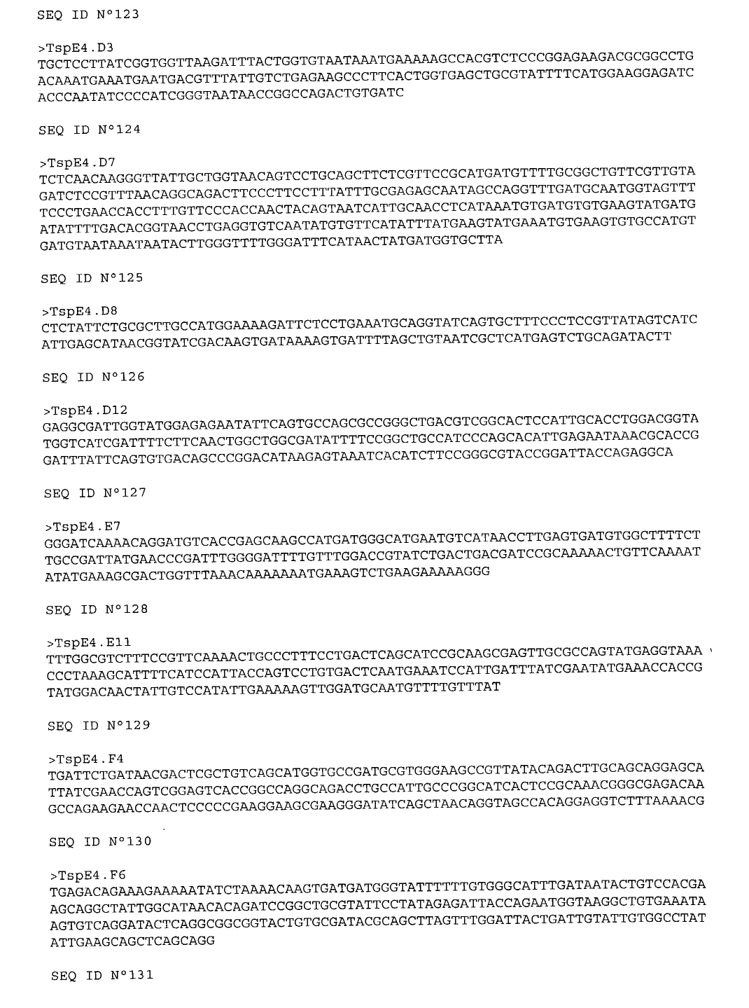

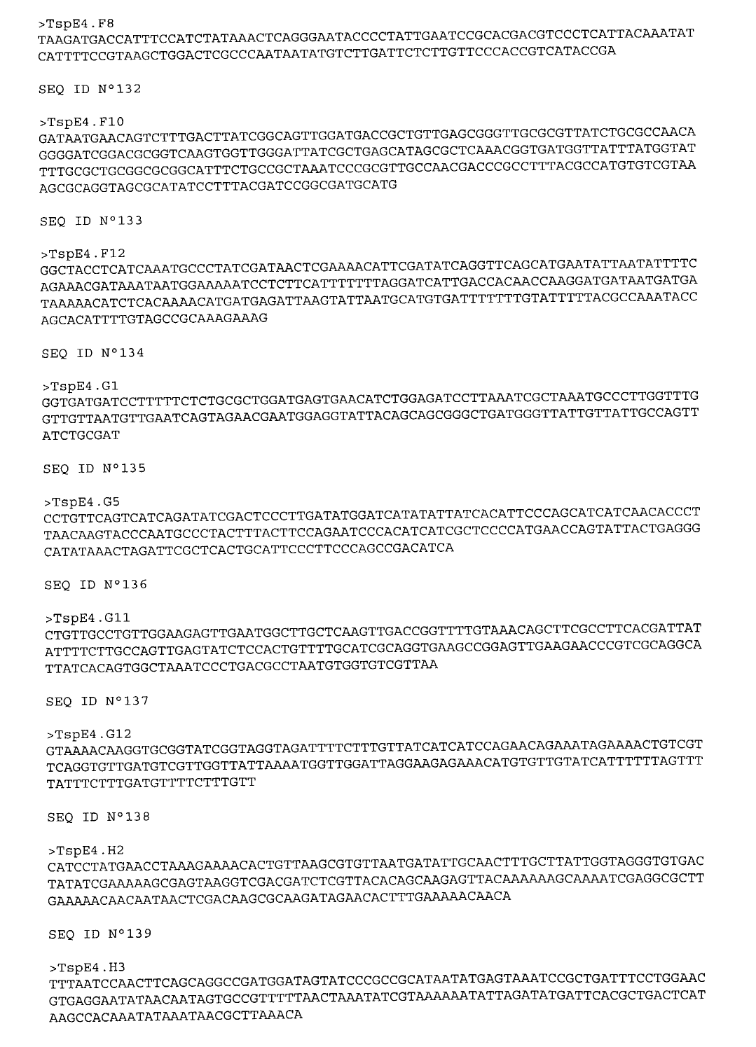

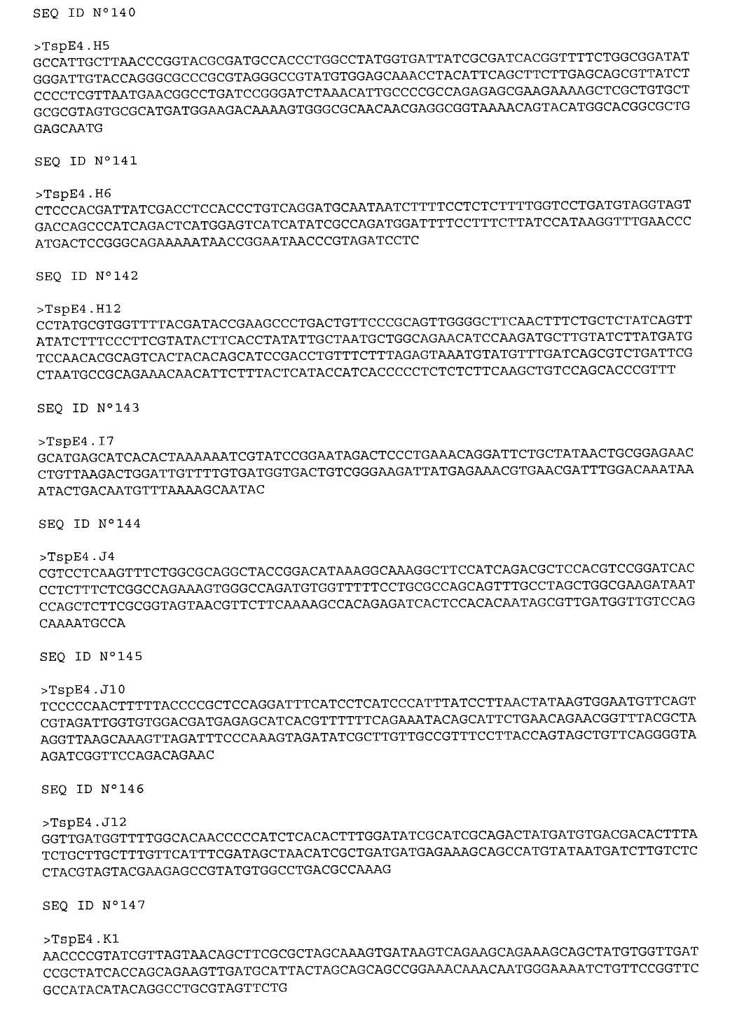

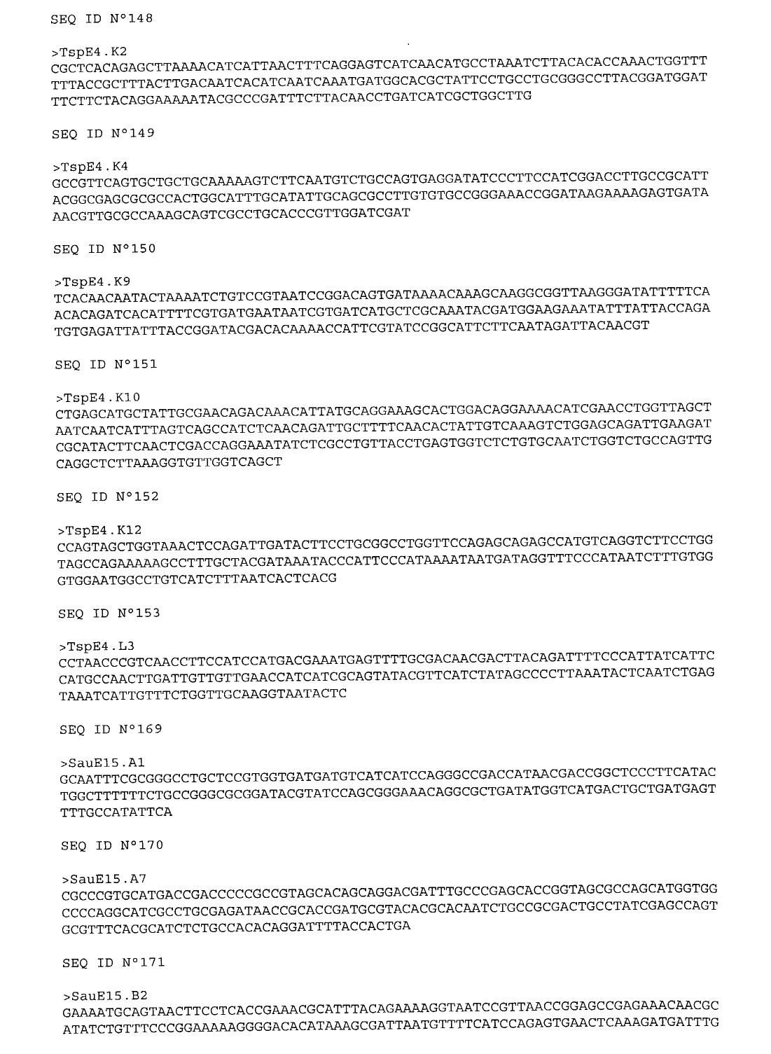

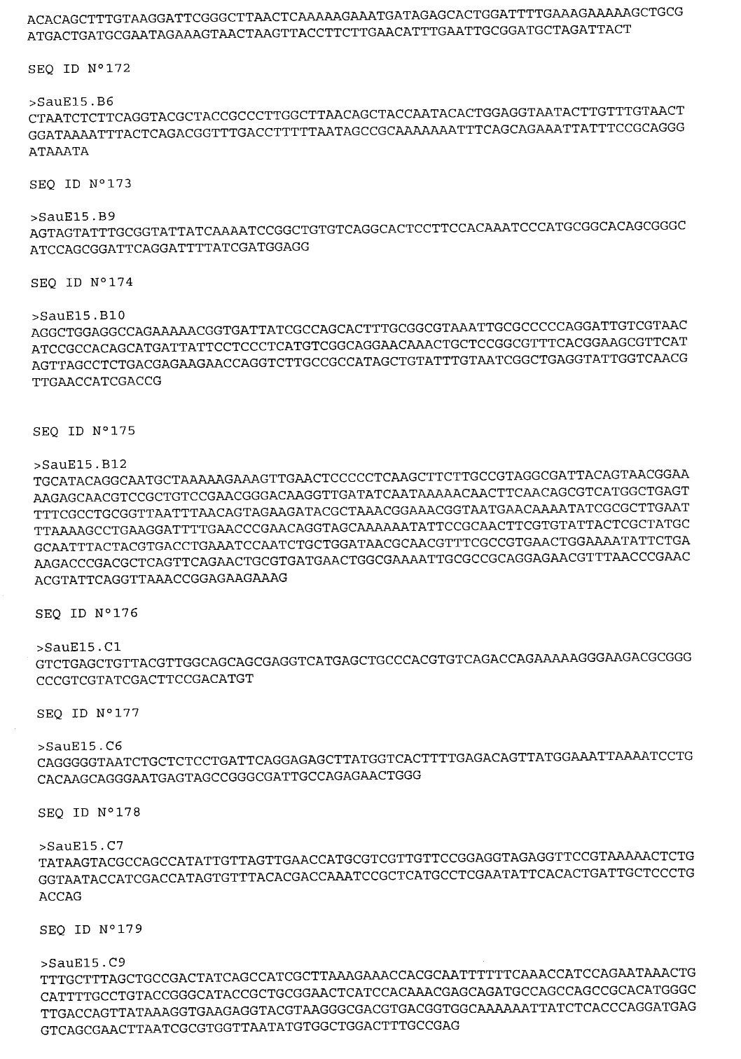

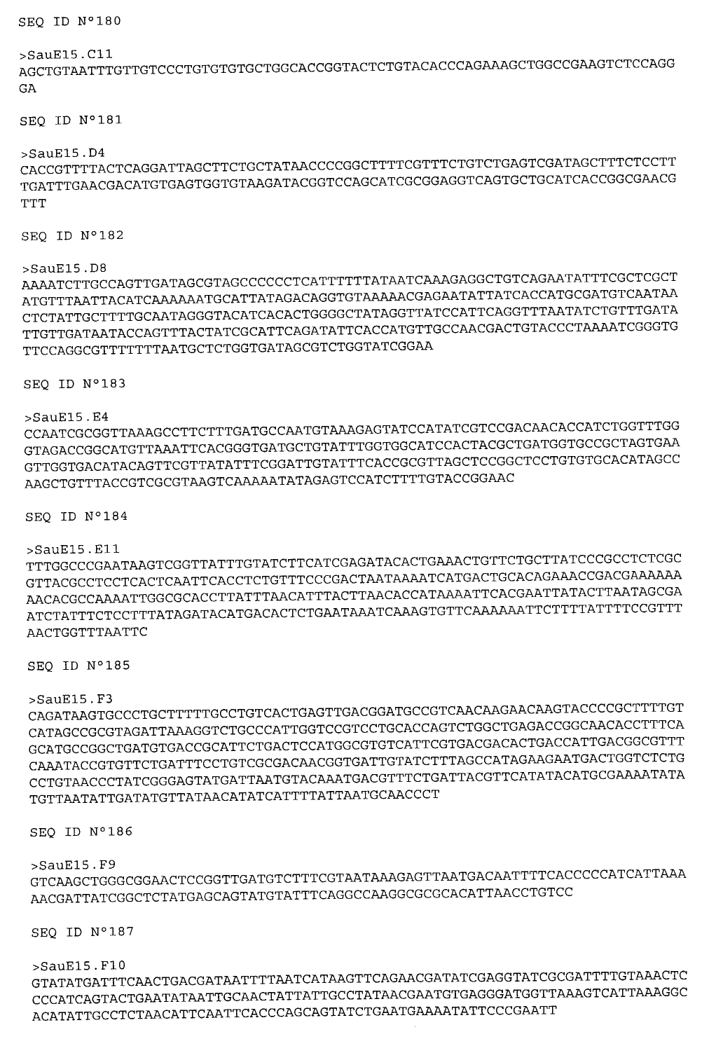

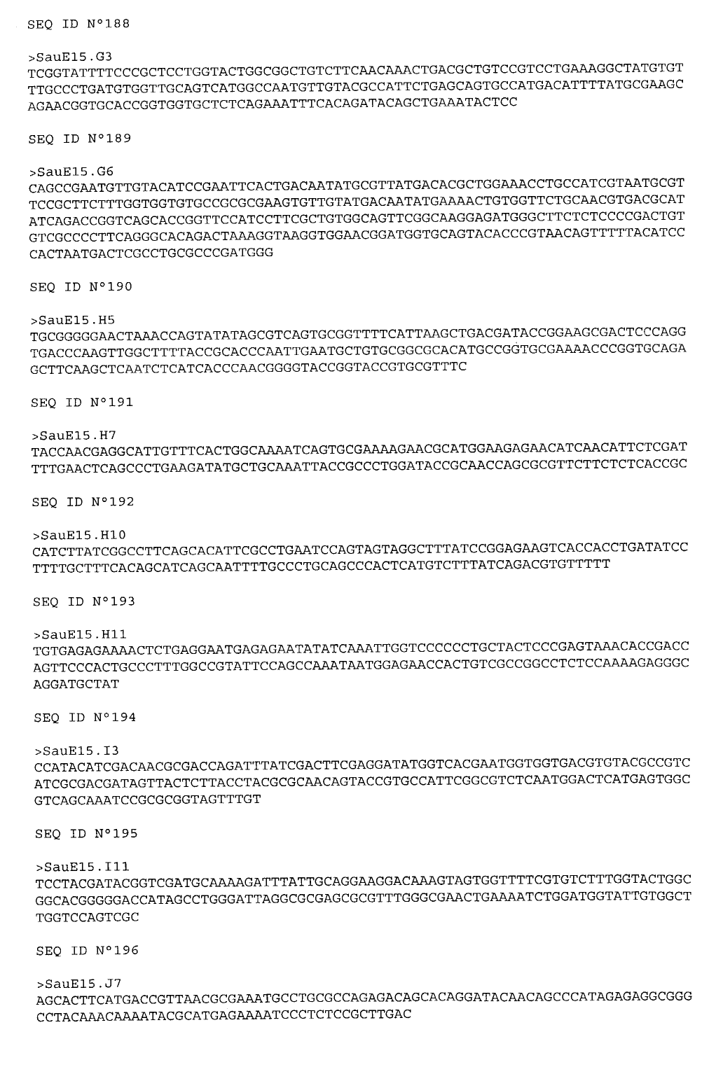

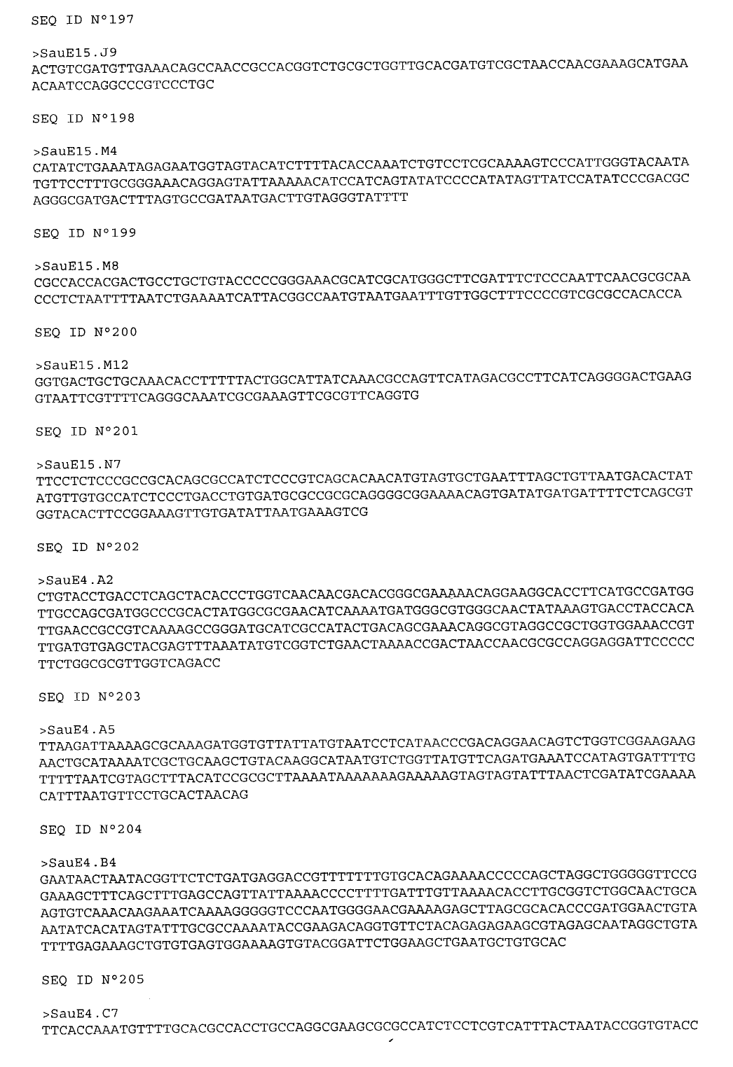

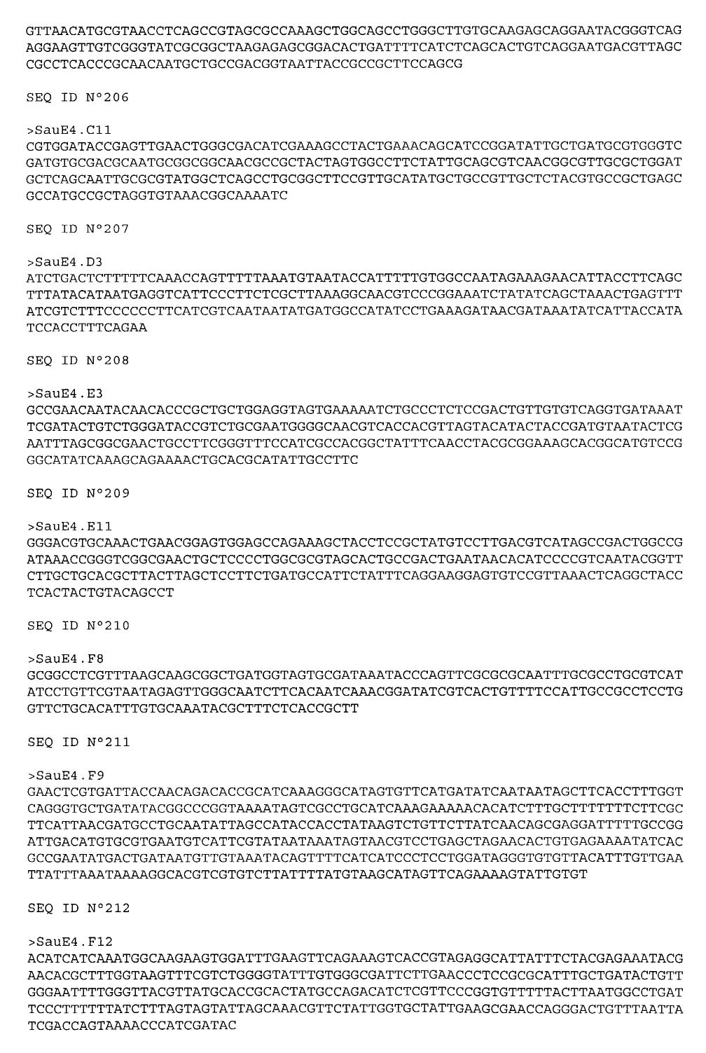

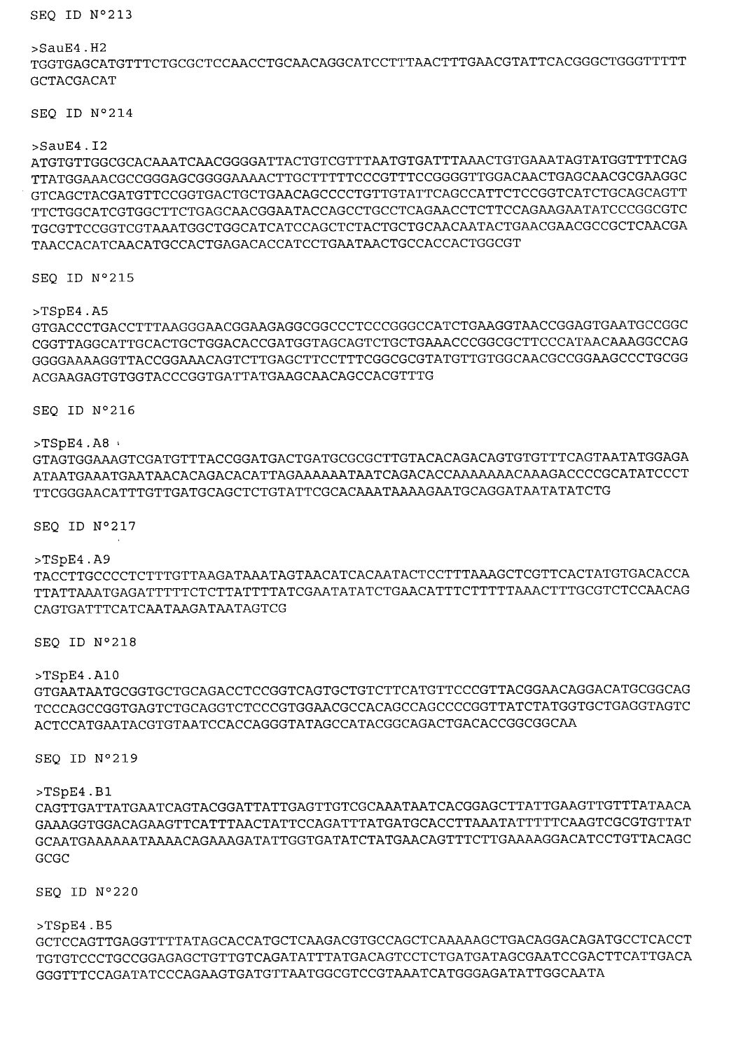

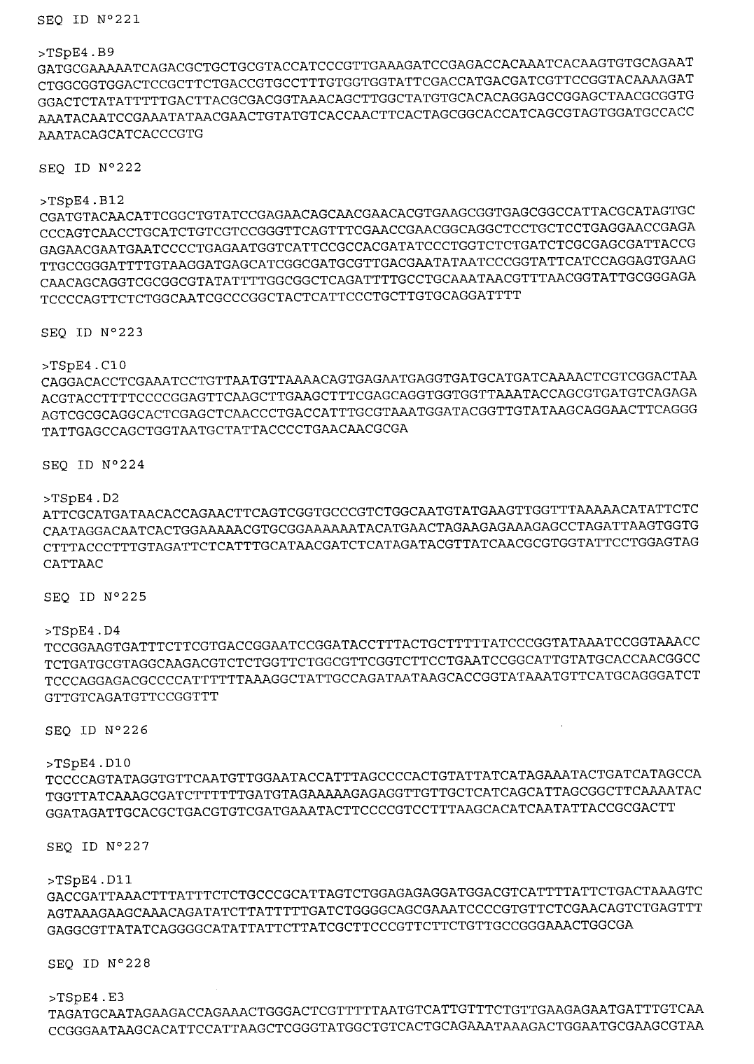

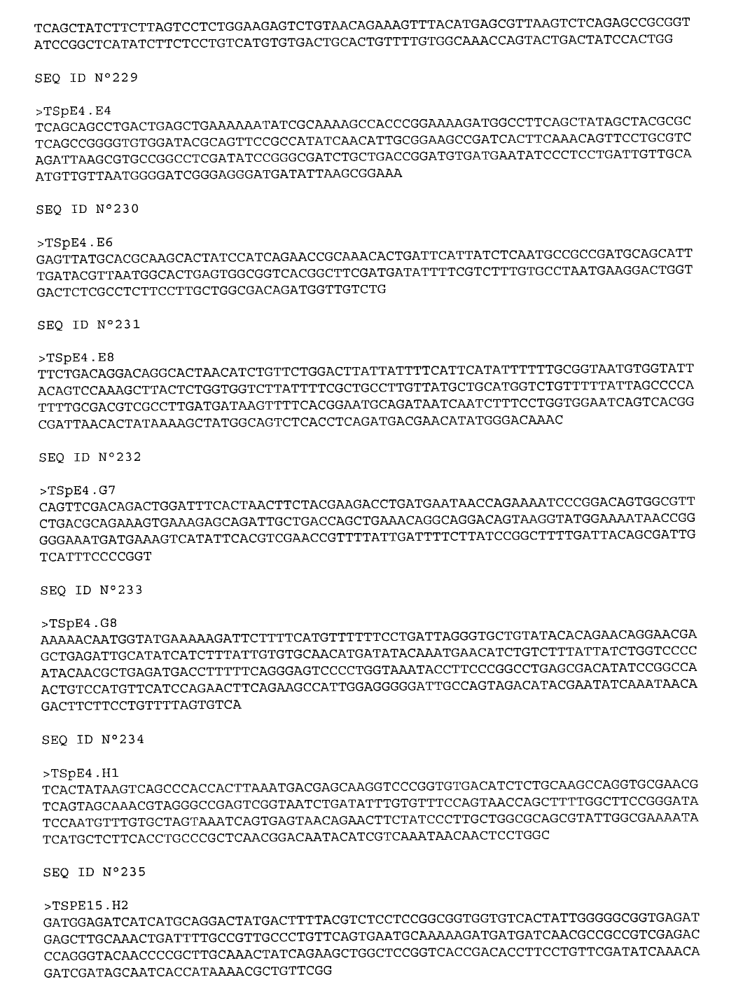

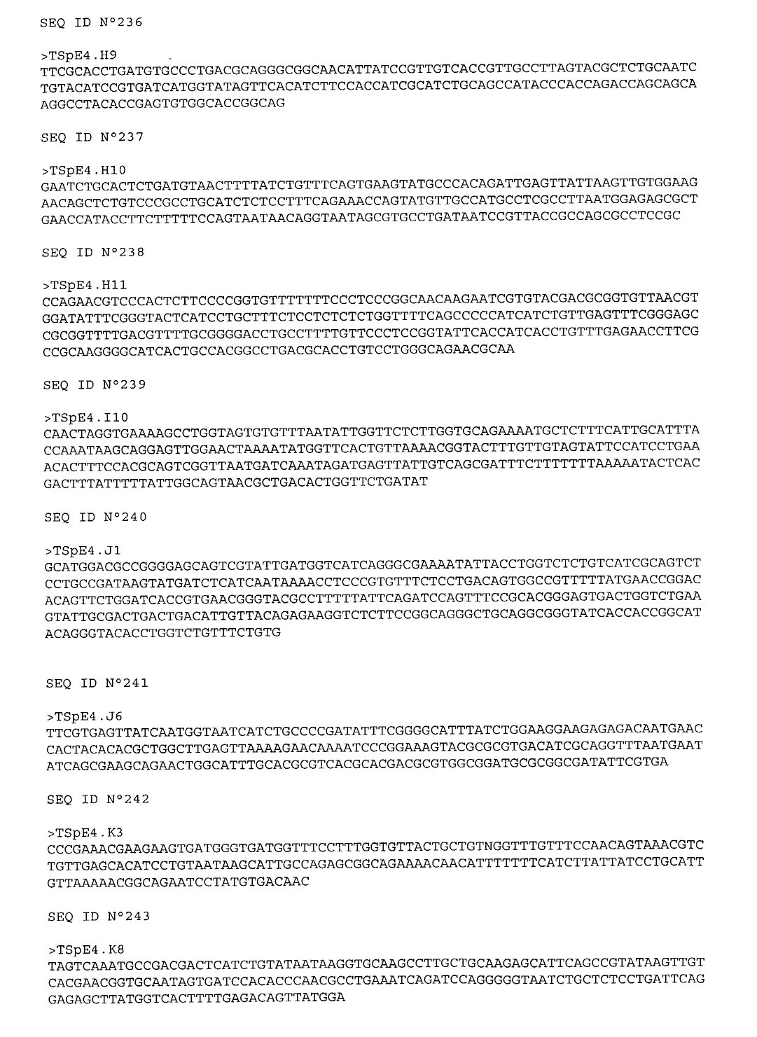

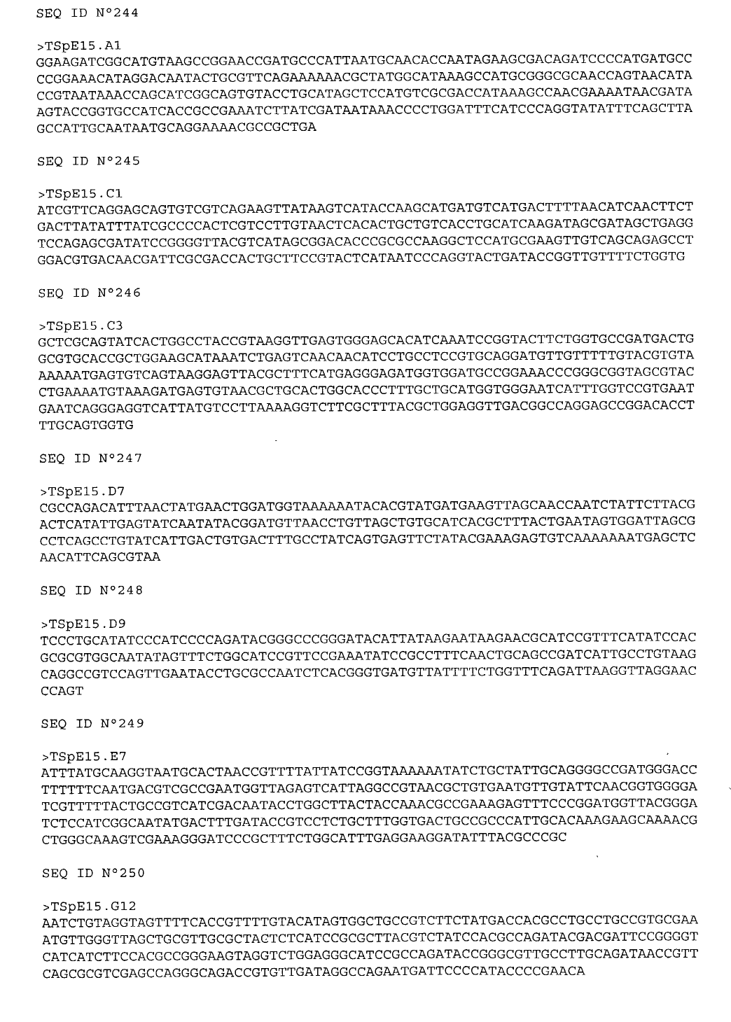

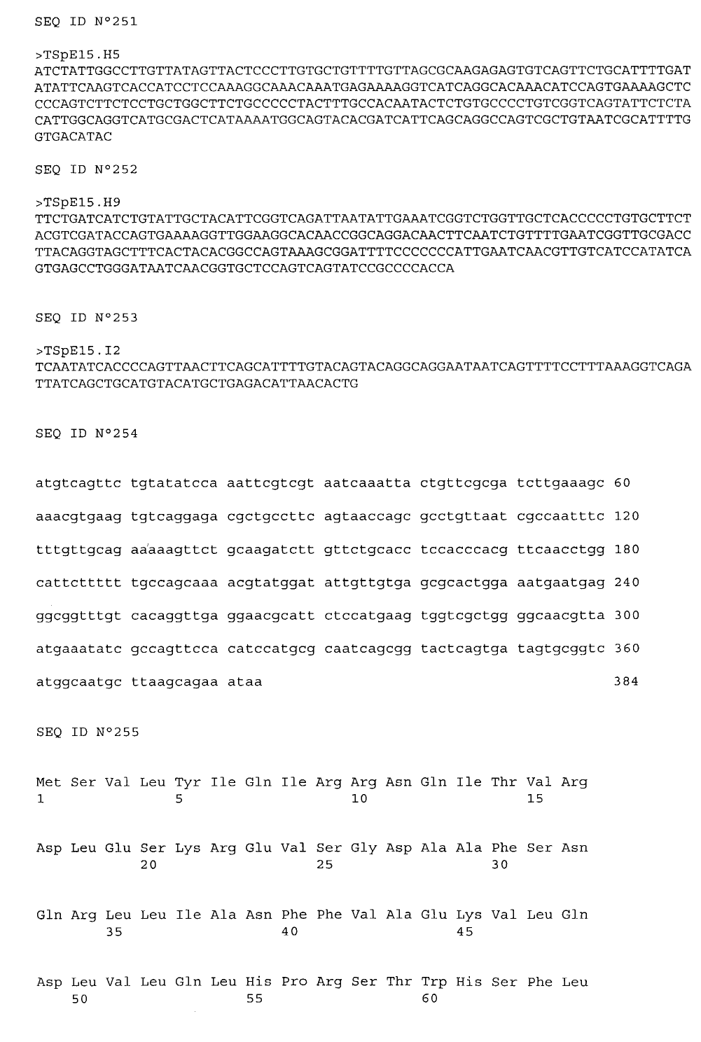

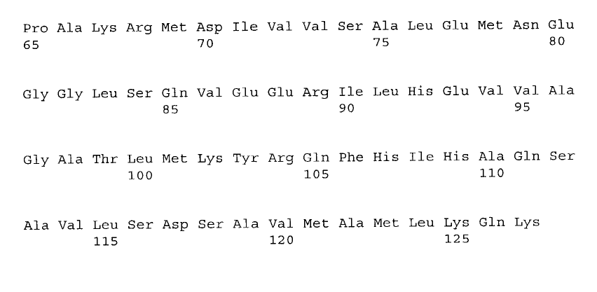

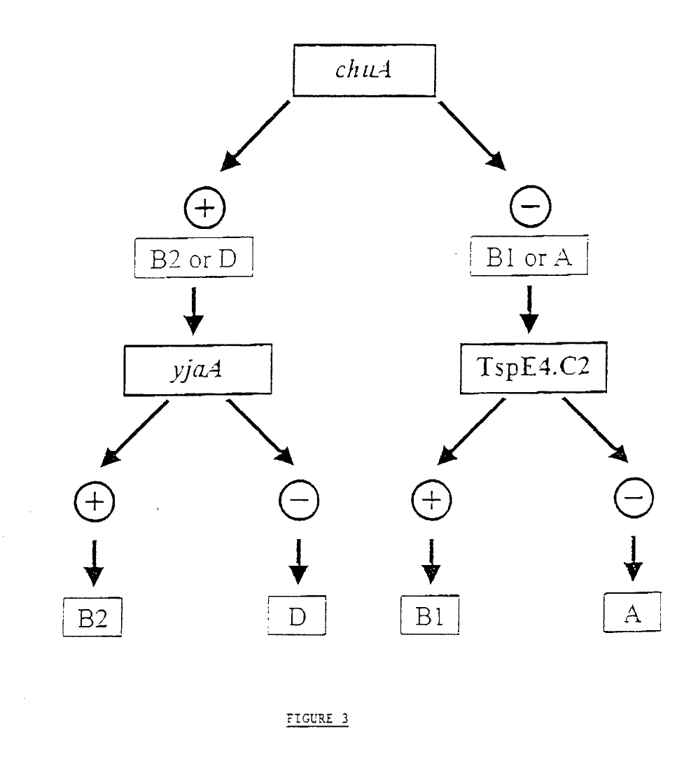

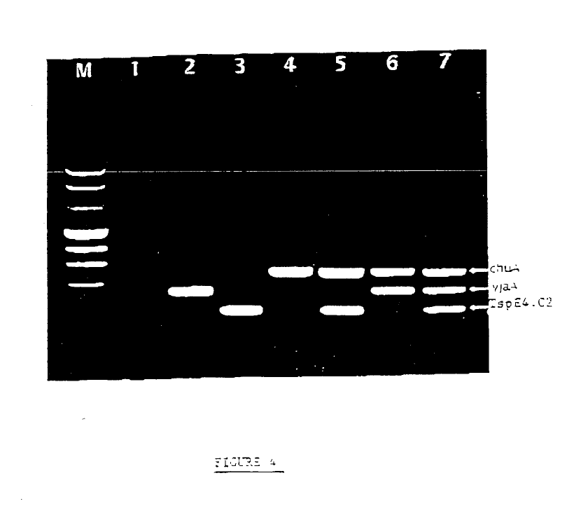

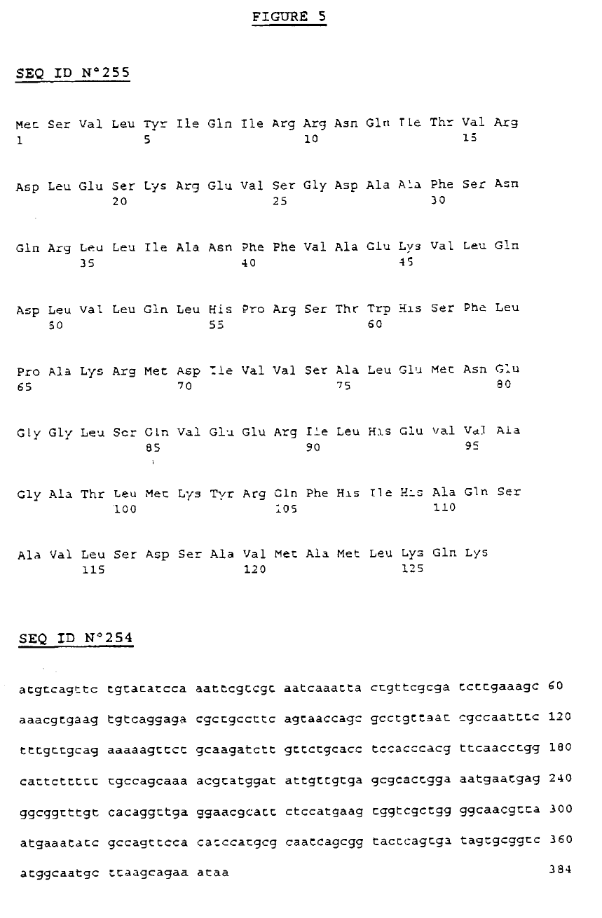

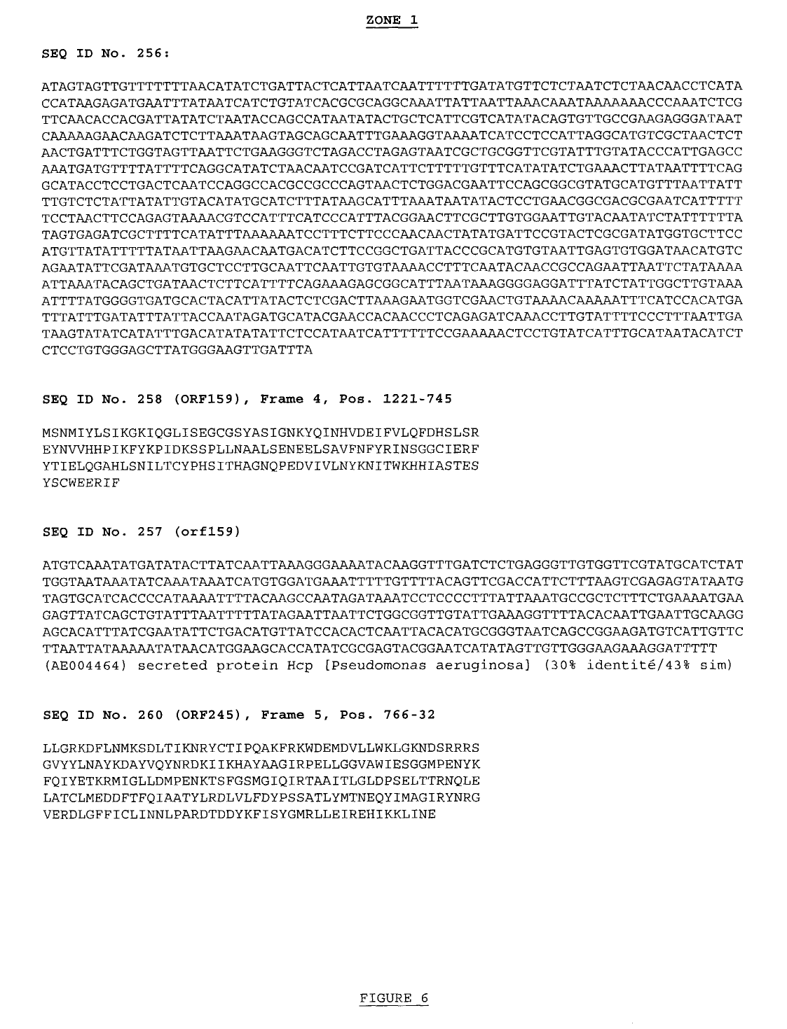

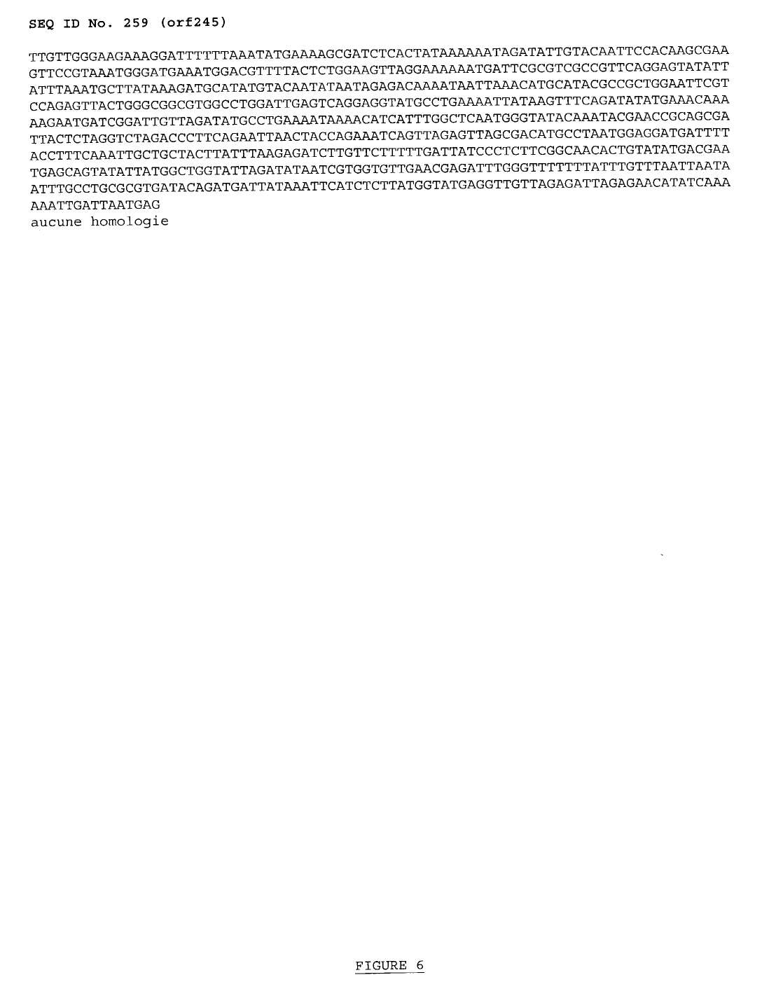

FIG. 2 gives the sequence listing for isolated B2/D+ A− fragments, and indicates their respective SEQ ID NOS,

FIG. 3 illustrates a decision diagram for the phylogenic analysis of an E. coli strain,

FIG. 4 represents the various phylogenic profiles obtained using a triplex PCR method according to the invention (lanes 1 and 2: group A; lane 3: group B1; lanes 4 and 5: group D; lanes 6 and 7: group B2),

FIG. 5 represents the polynucleotide sequence of the yjaA gene (SEQ ID NO: 254) and the corresponding ORF sequence,

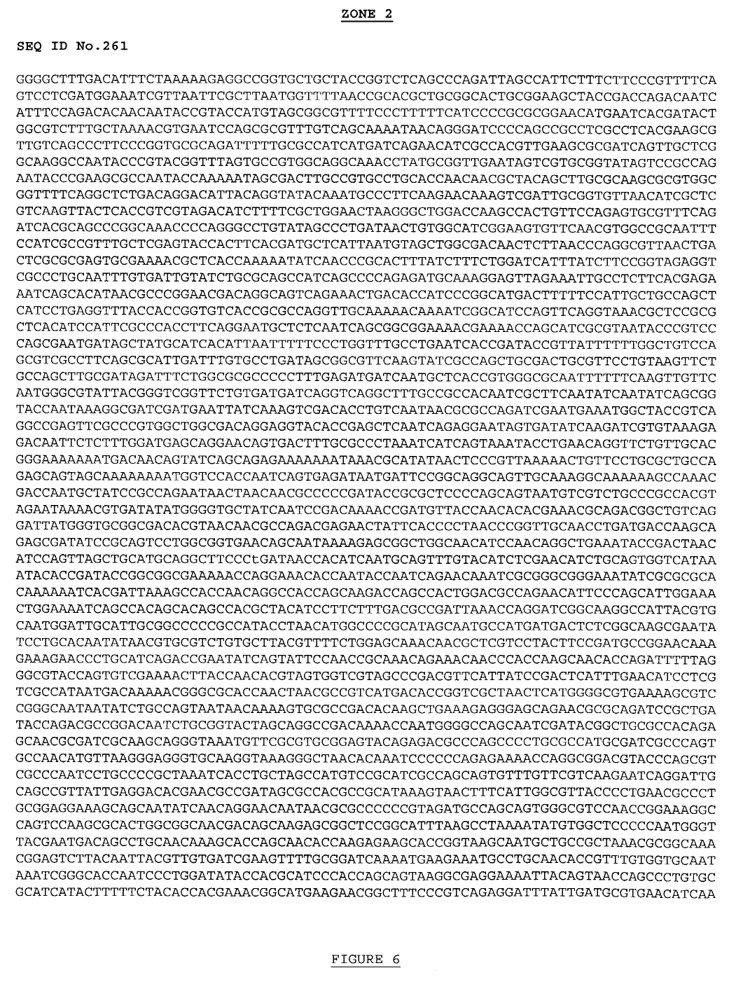

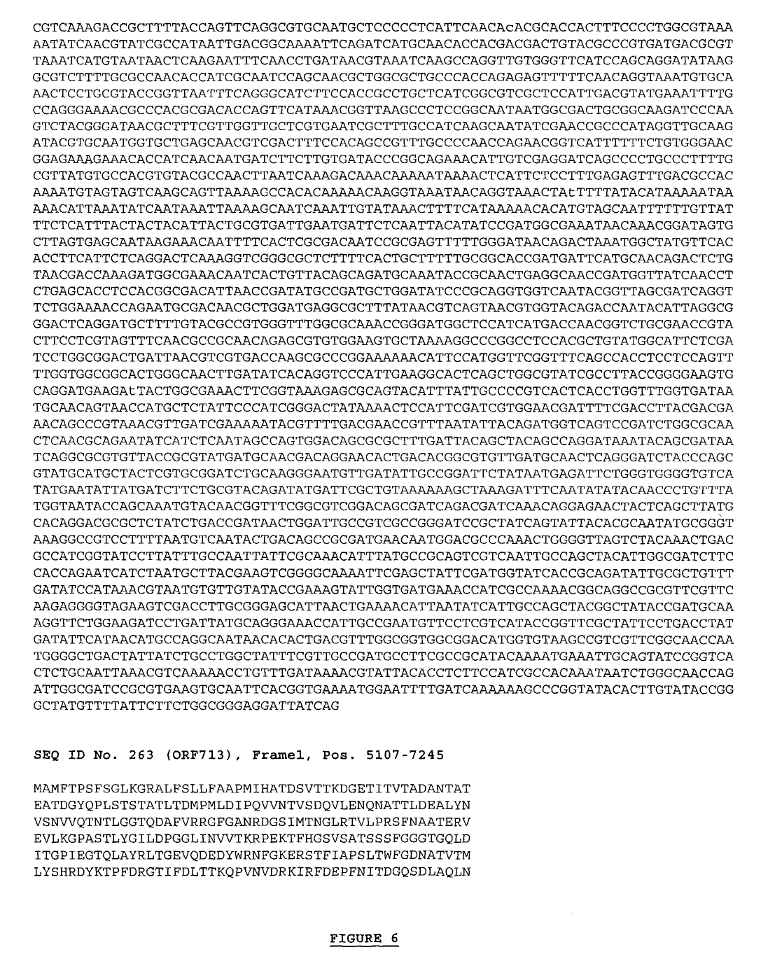

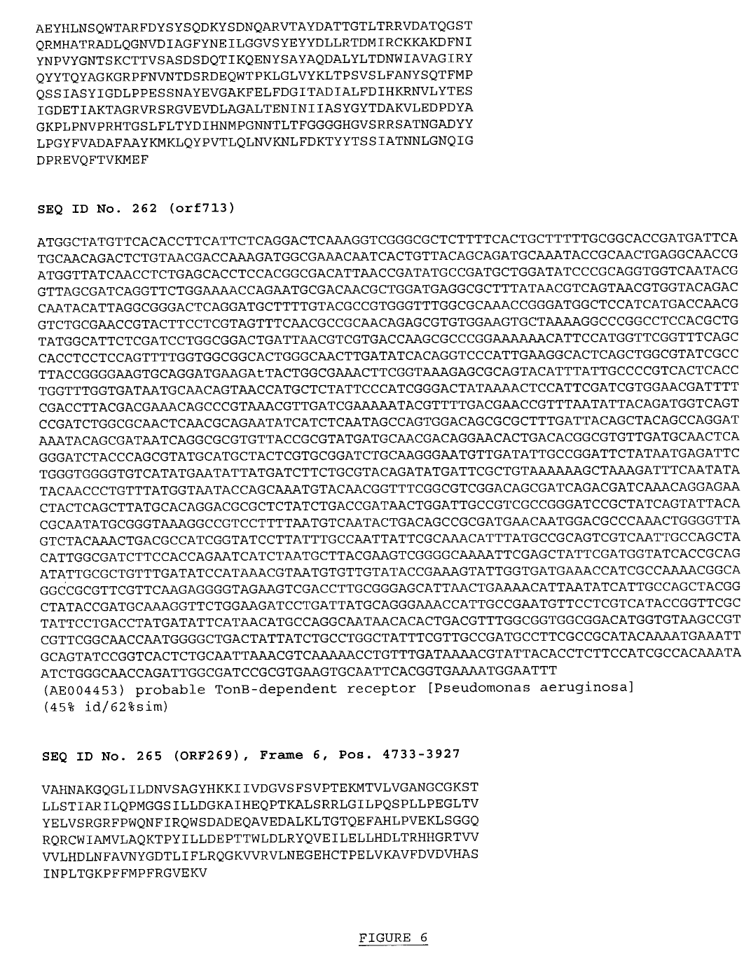

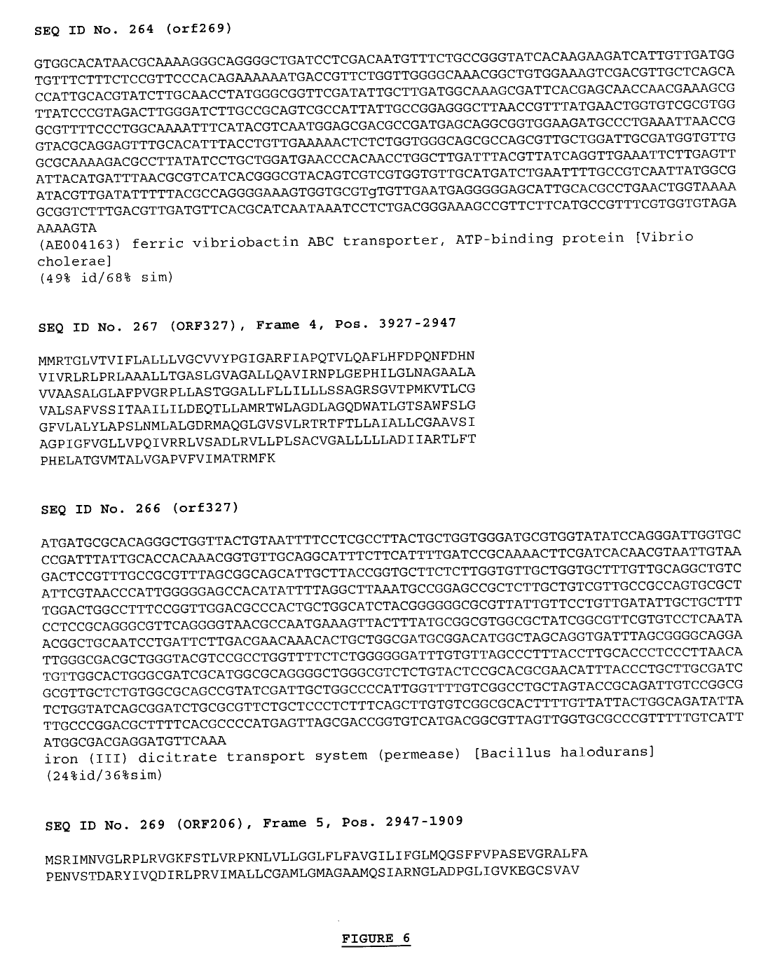

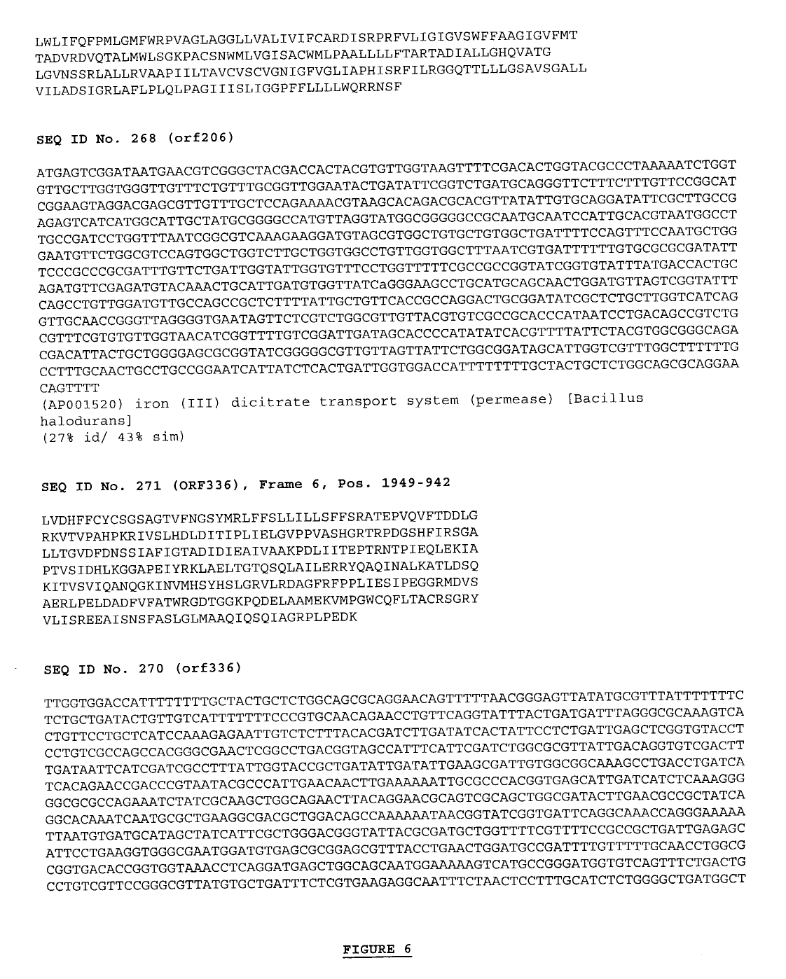

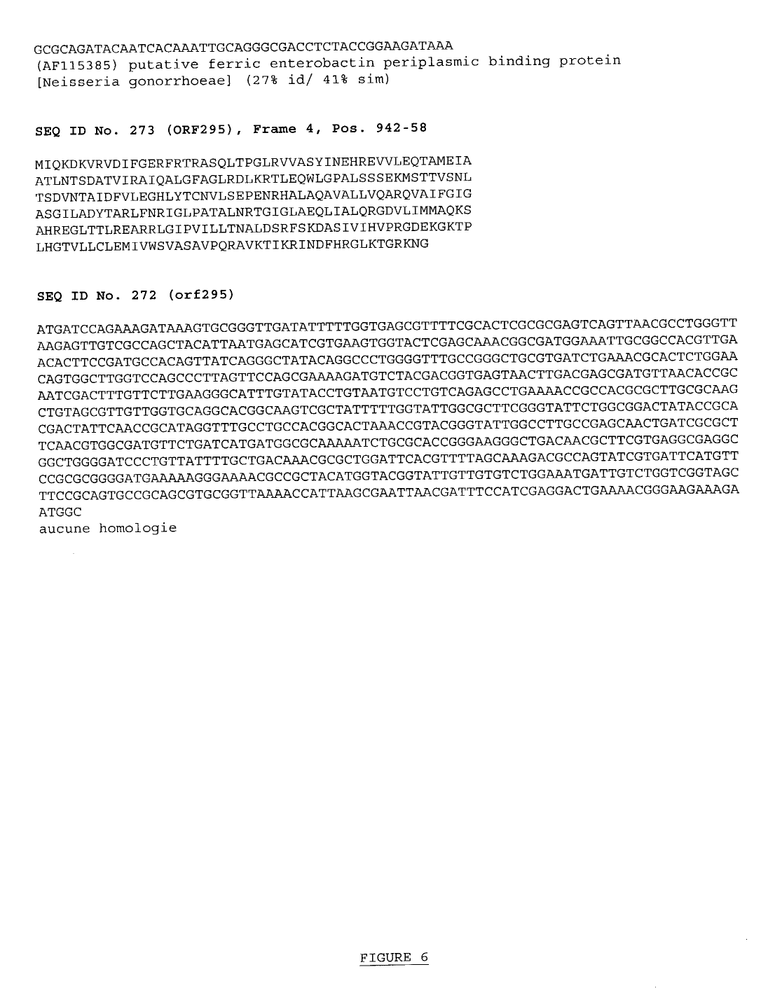

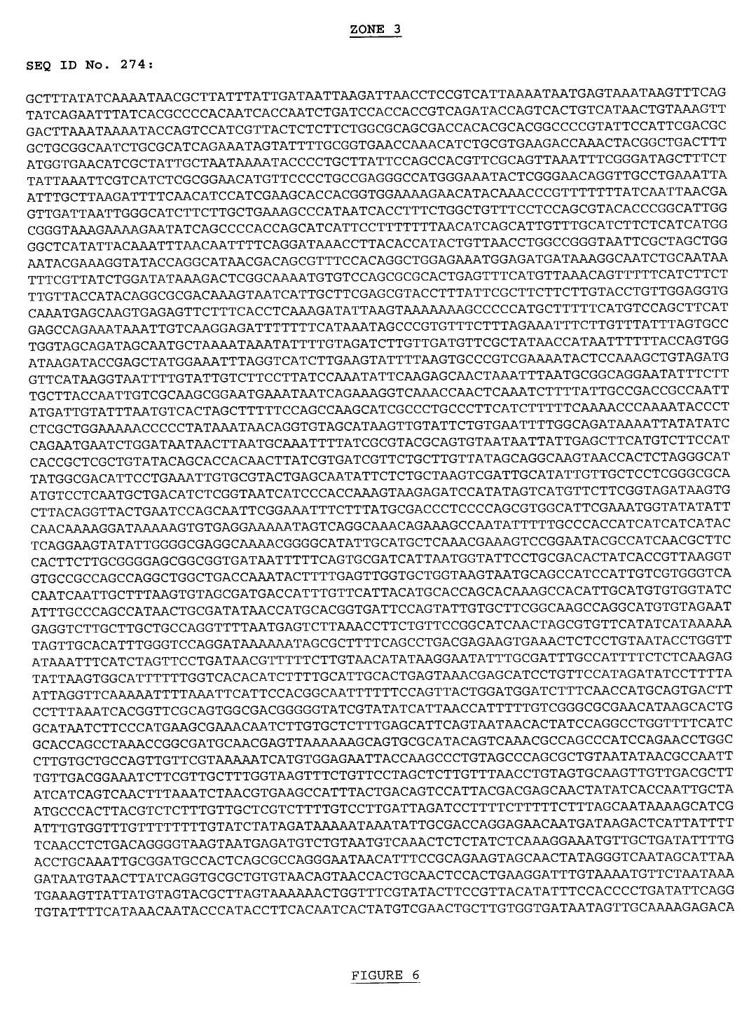

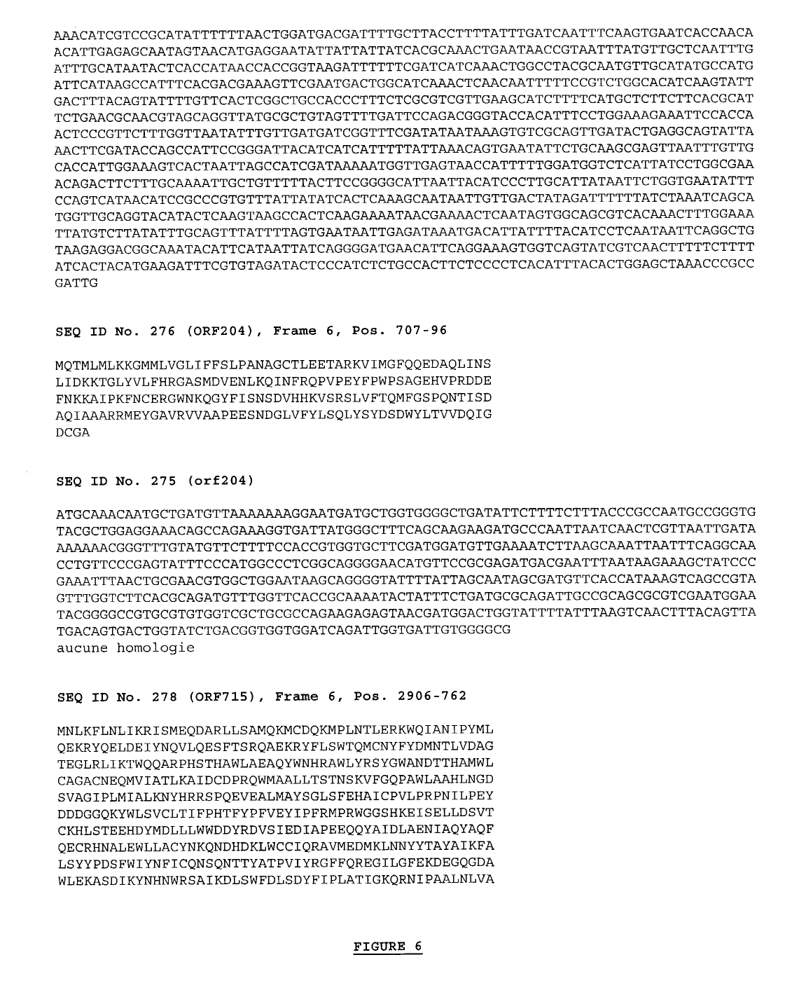

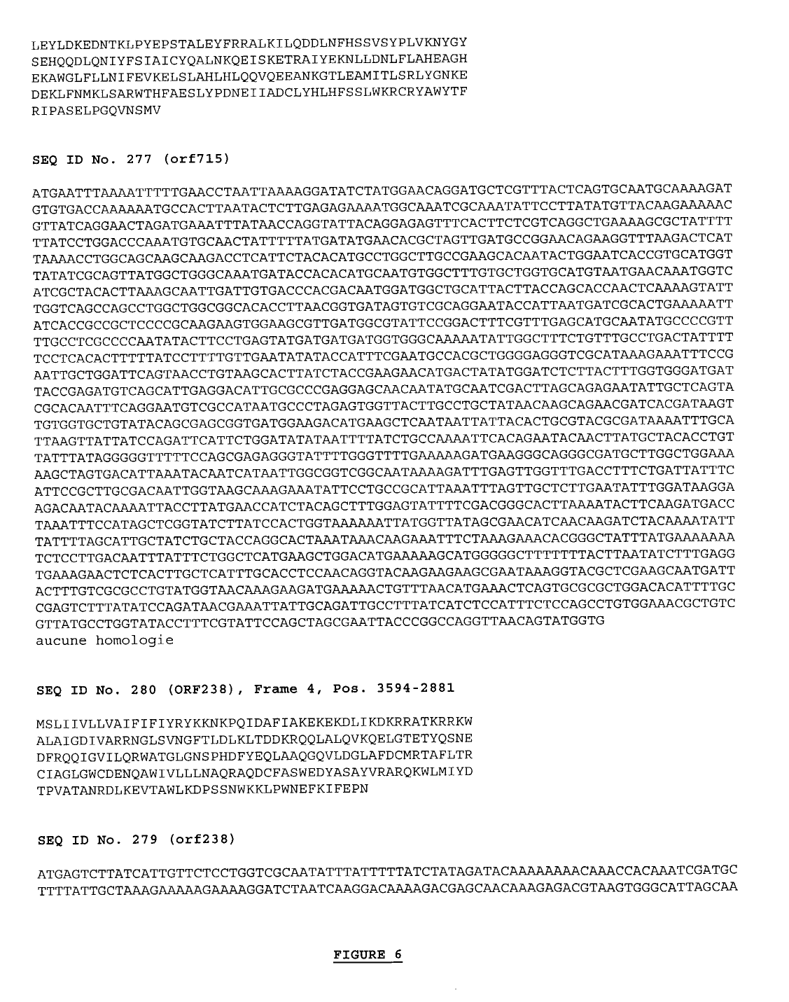

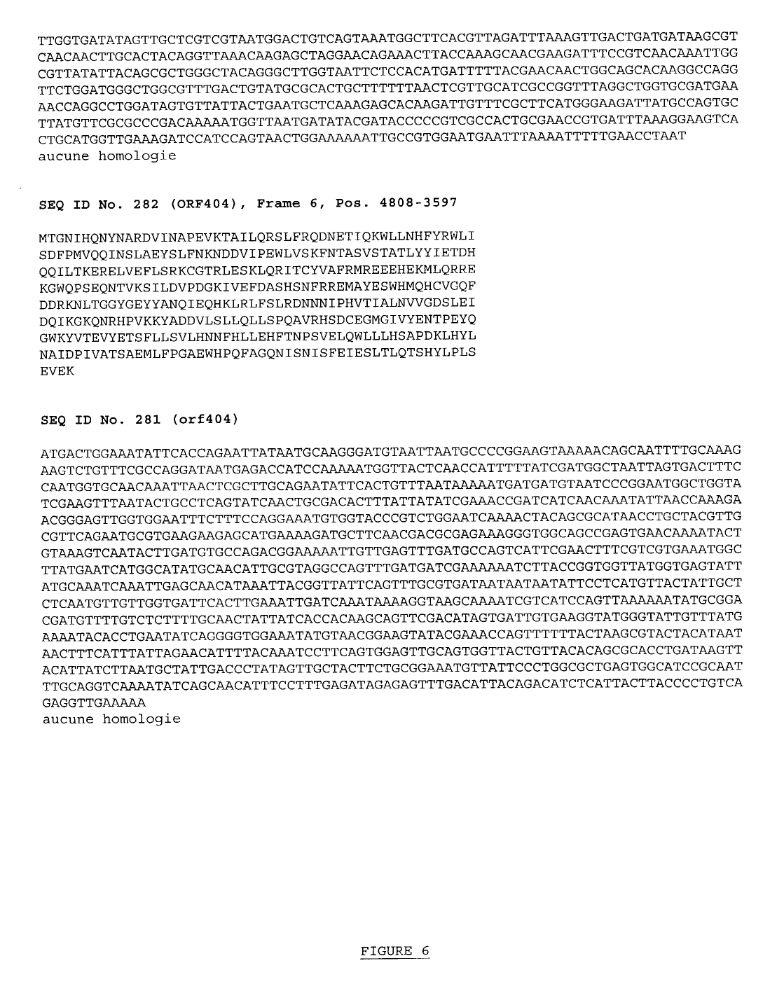

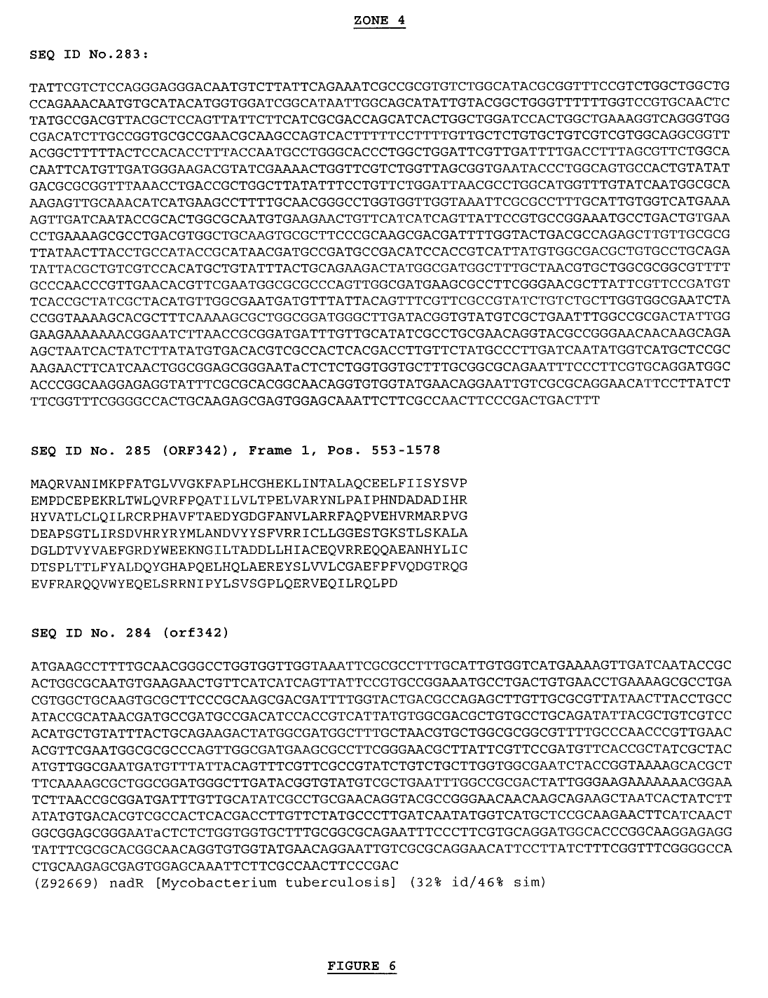

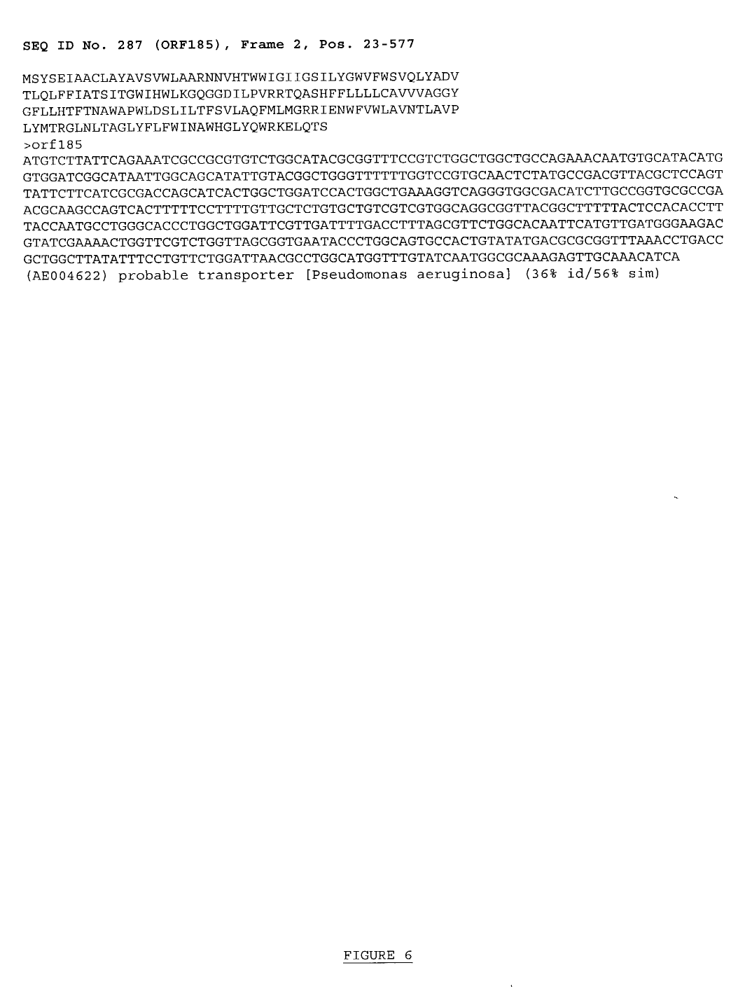

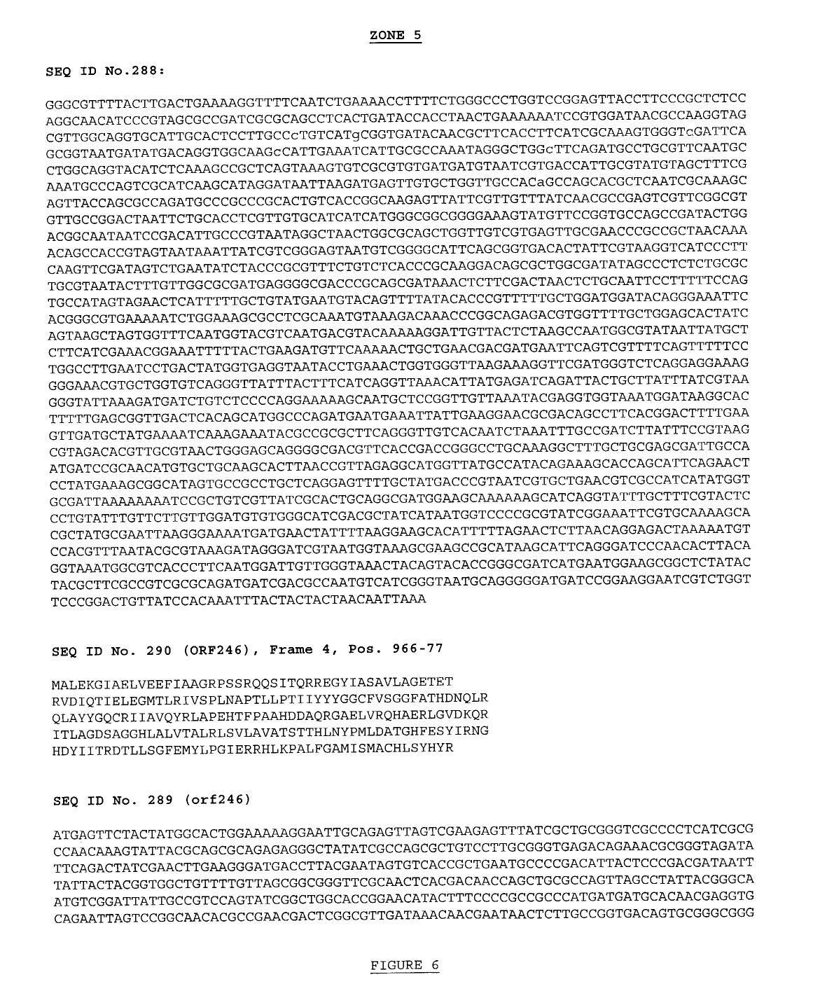

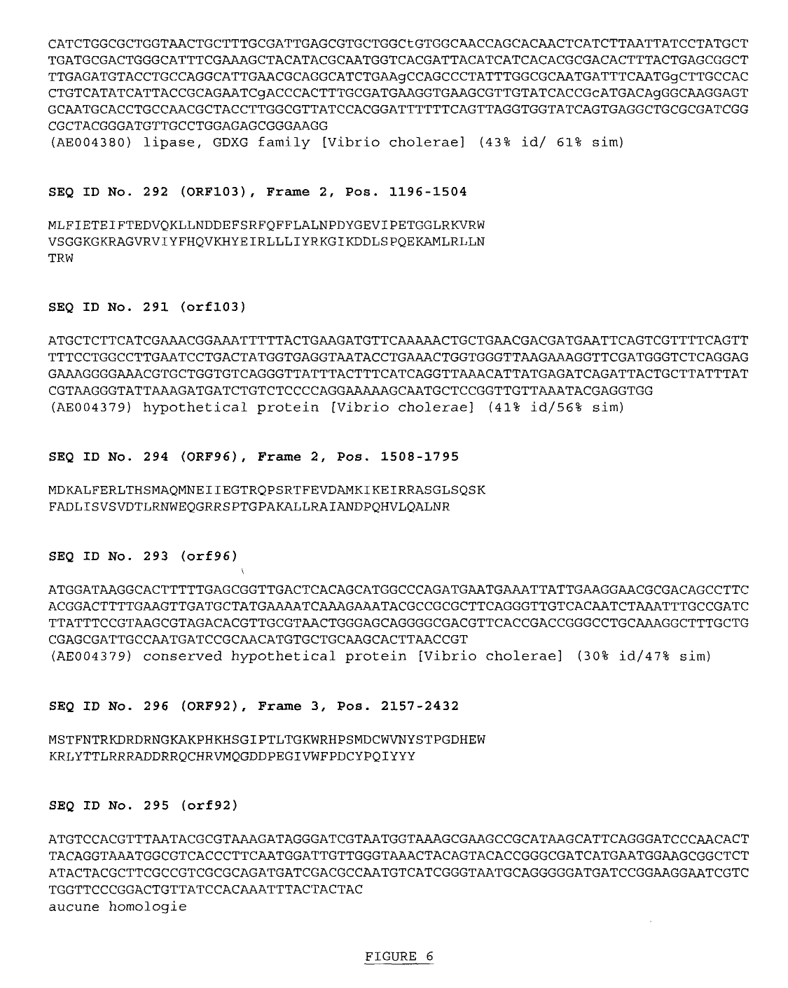

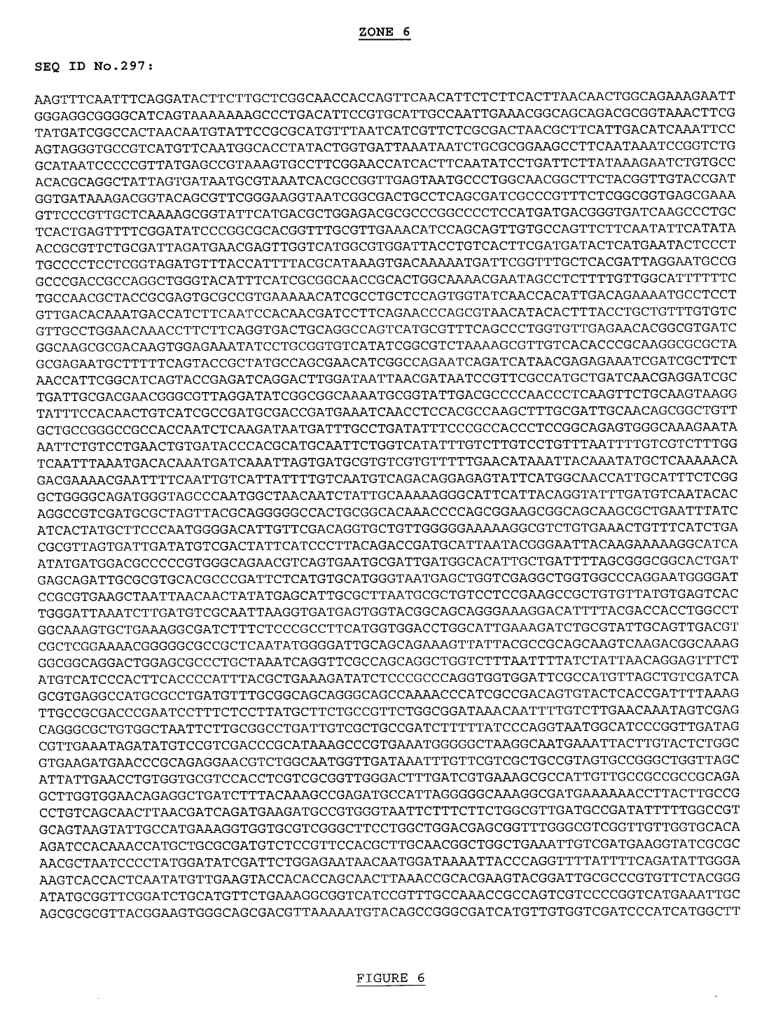

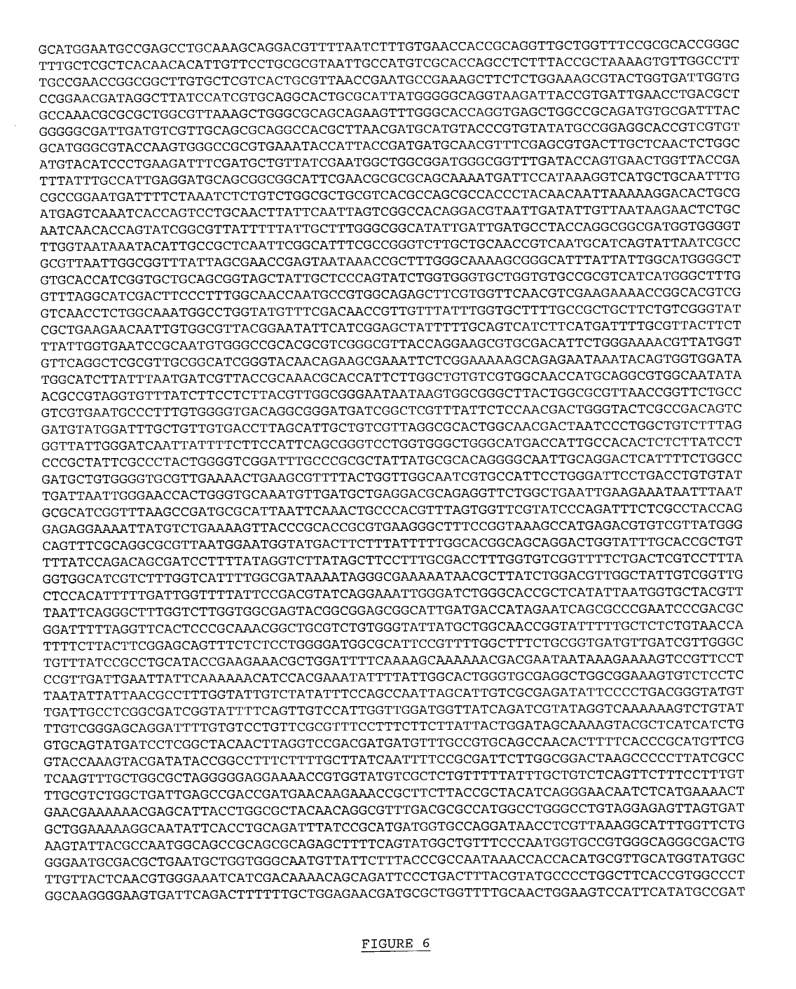

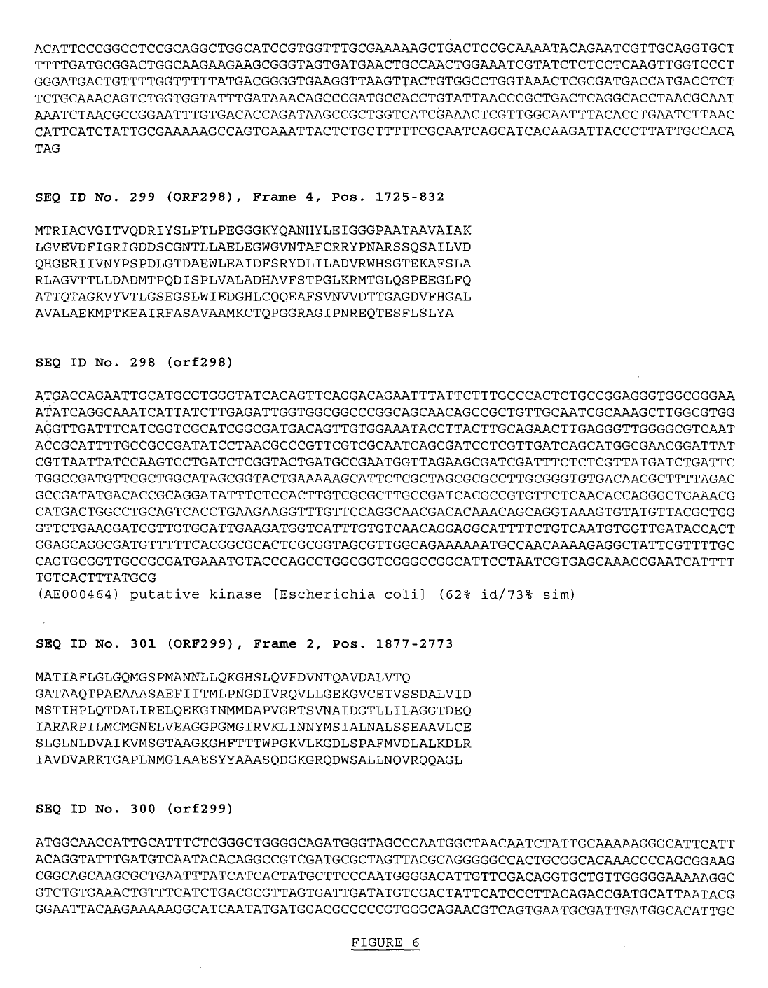

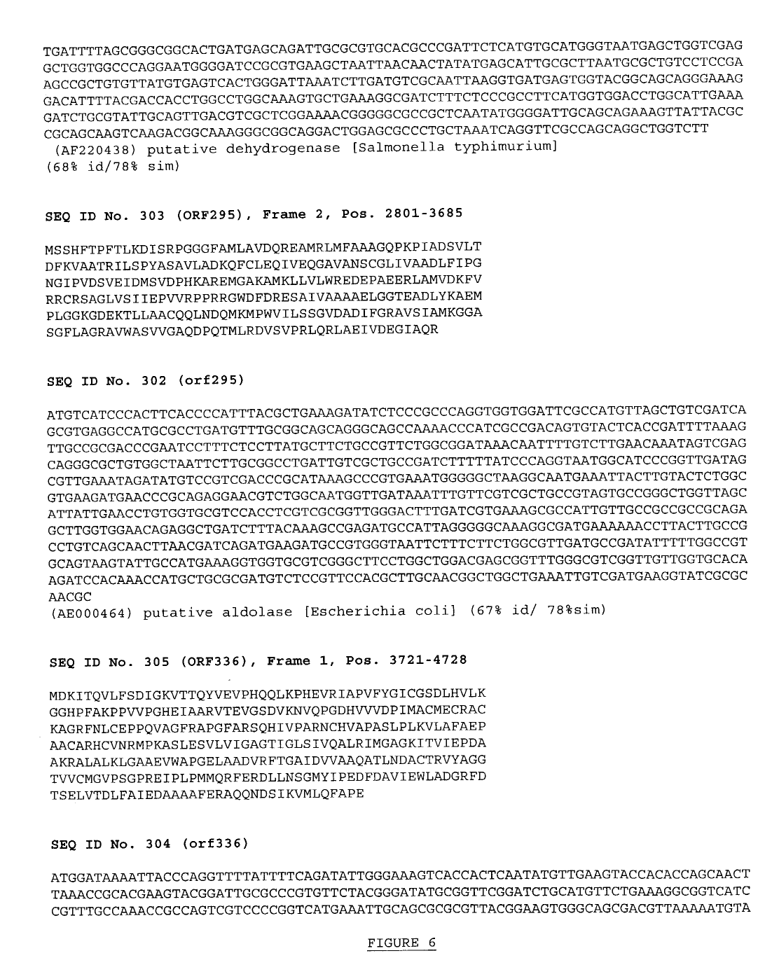

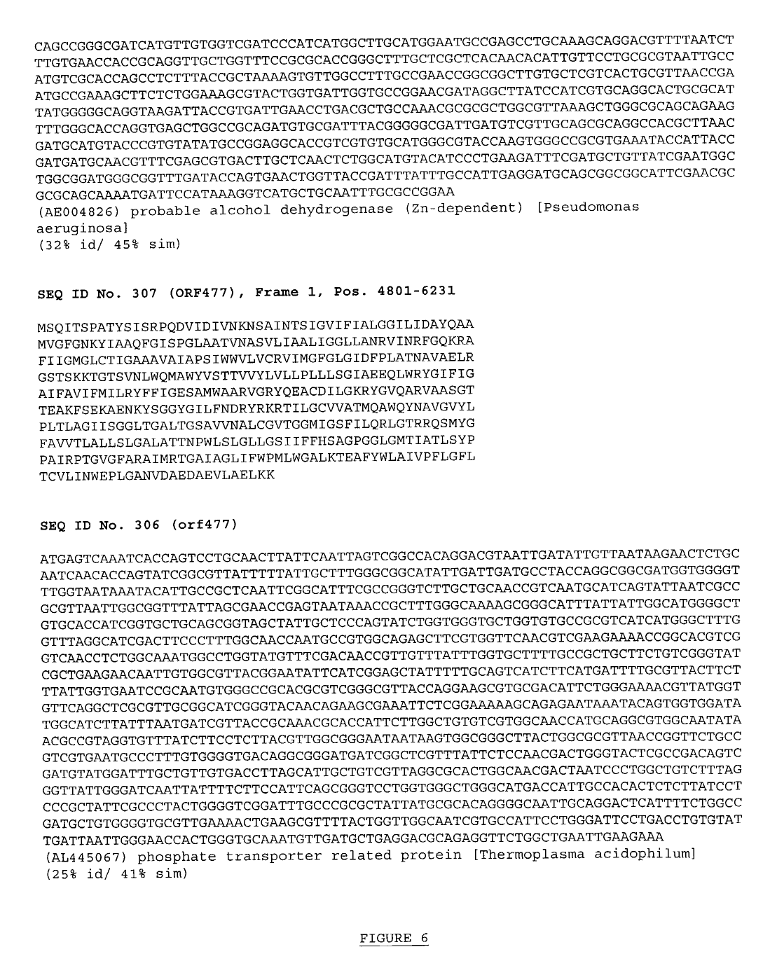

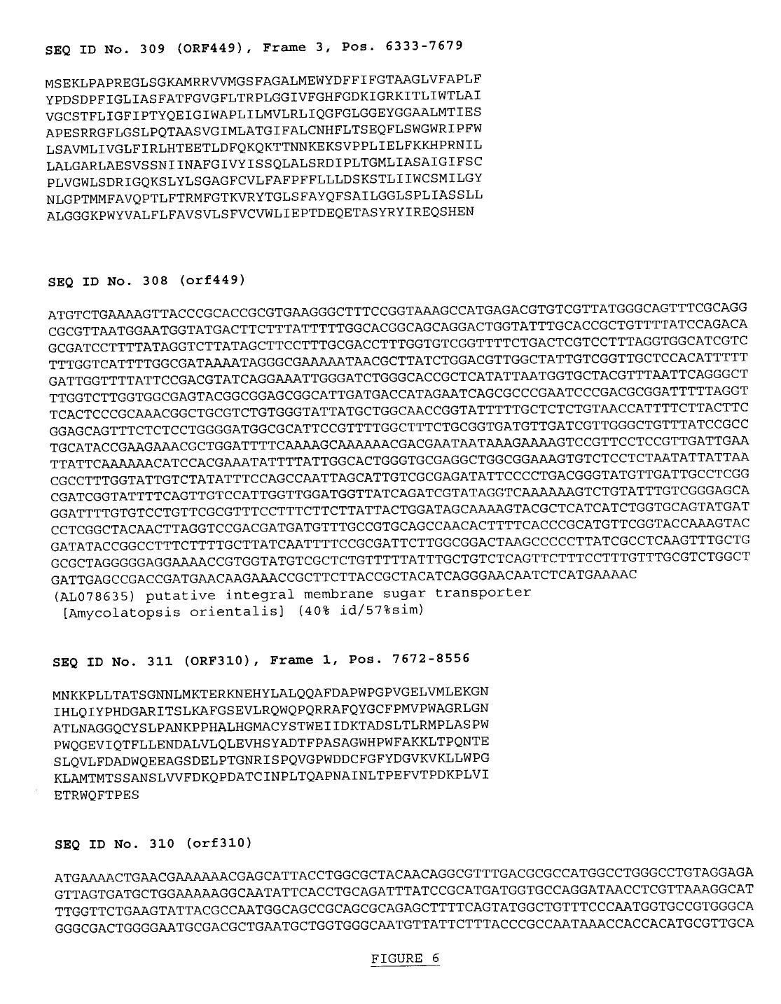

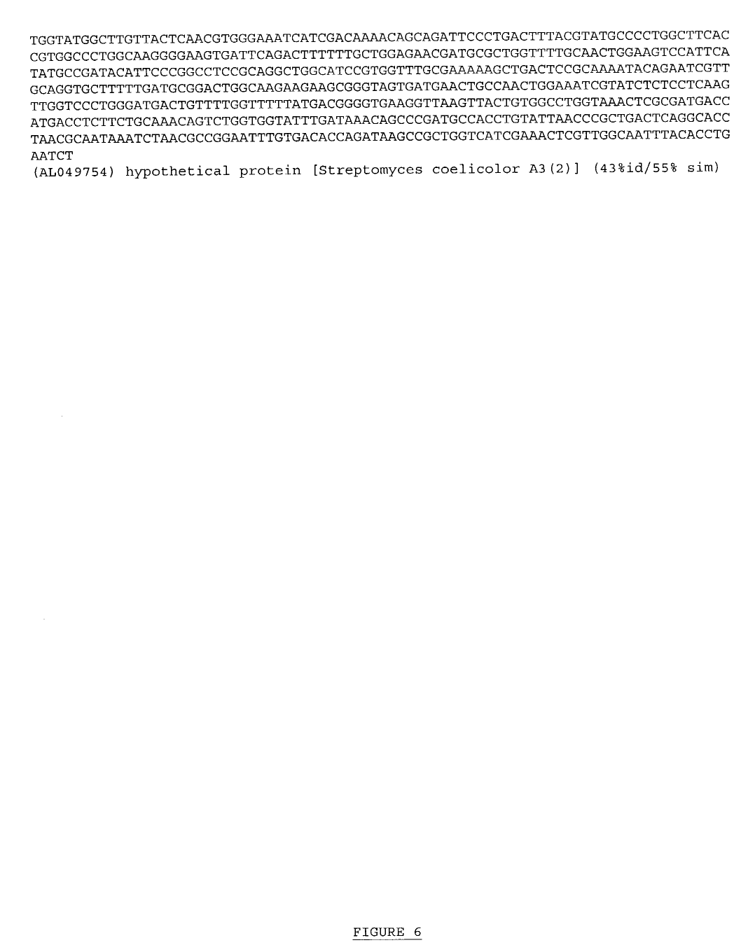

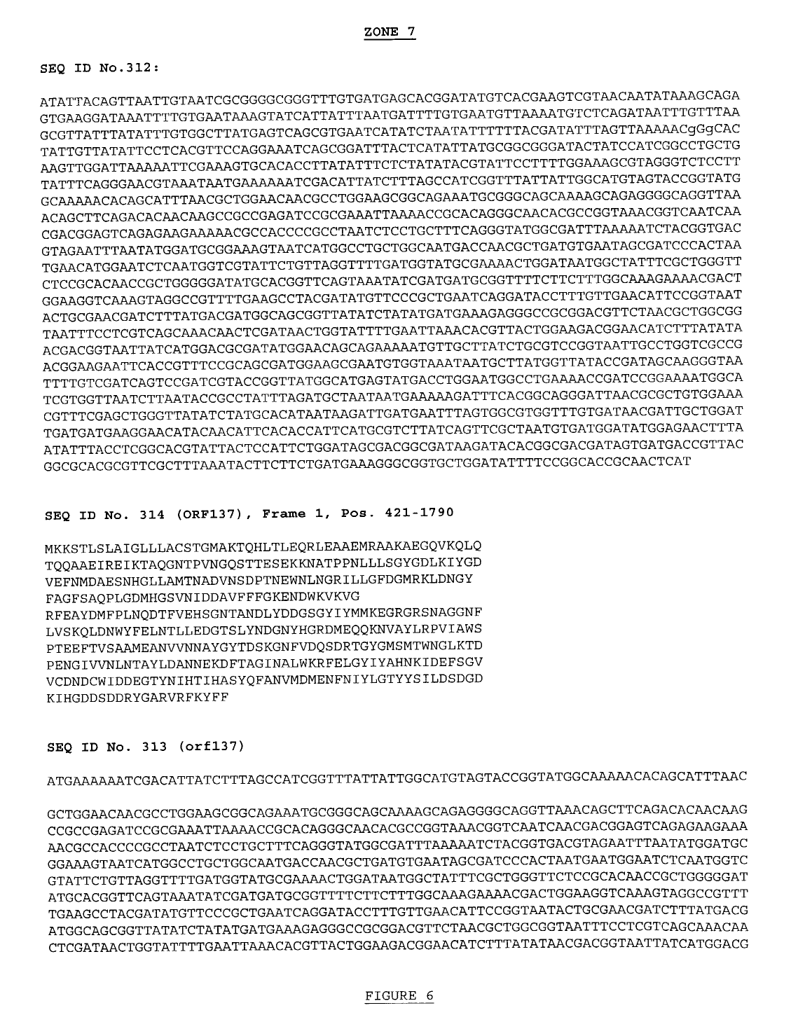

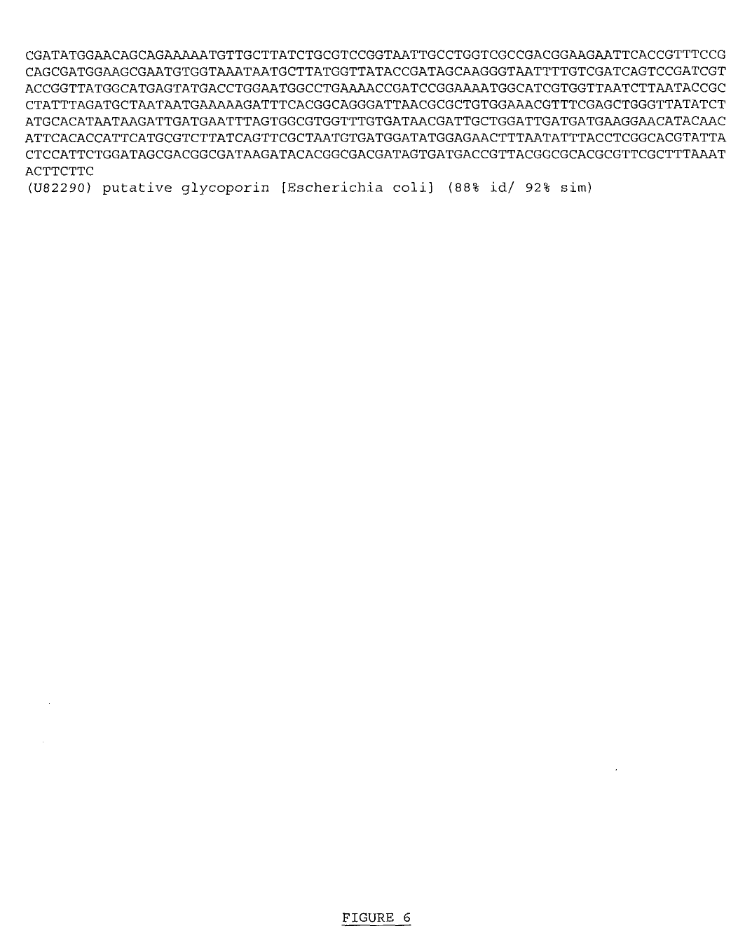

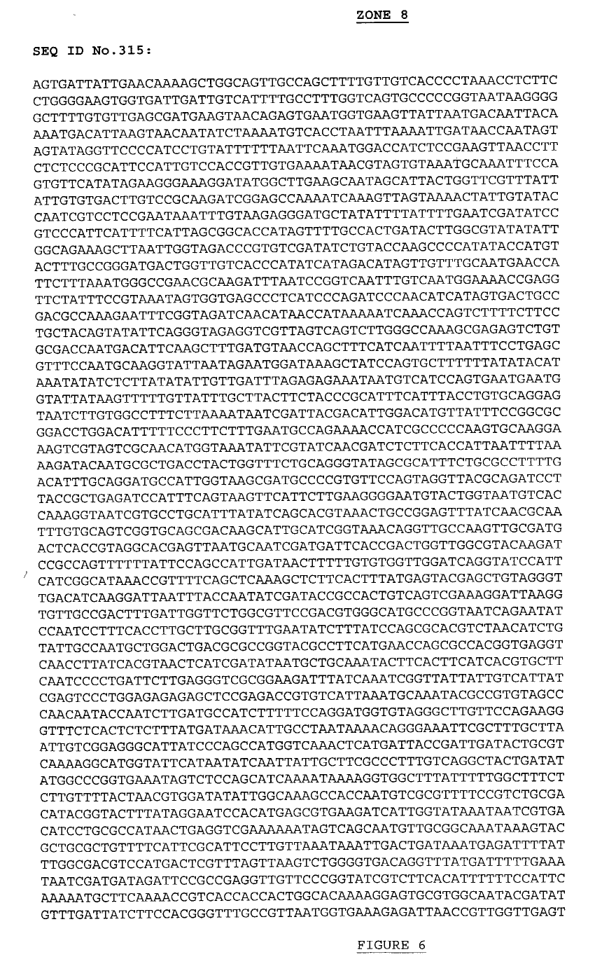

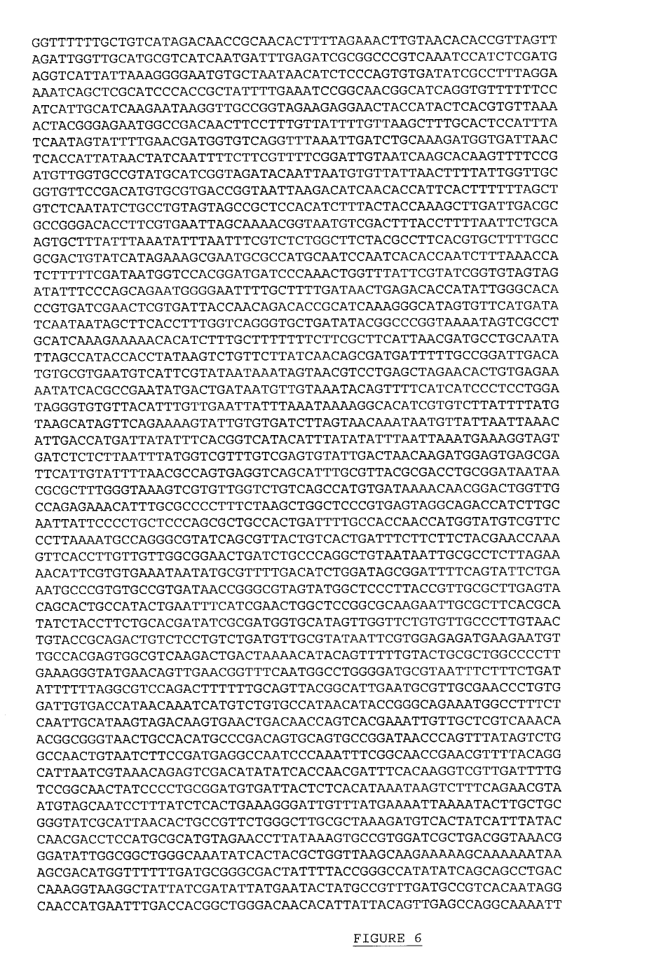

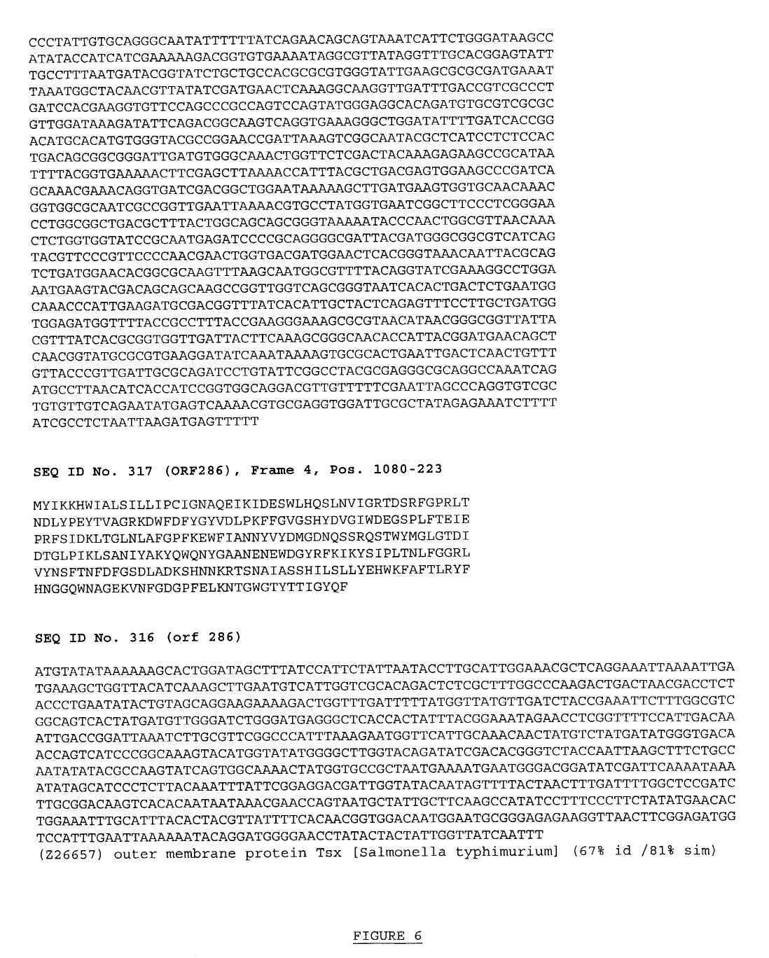

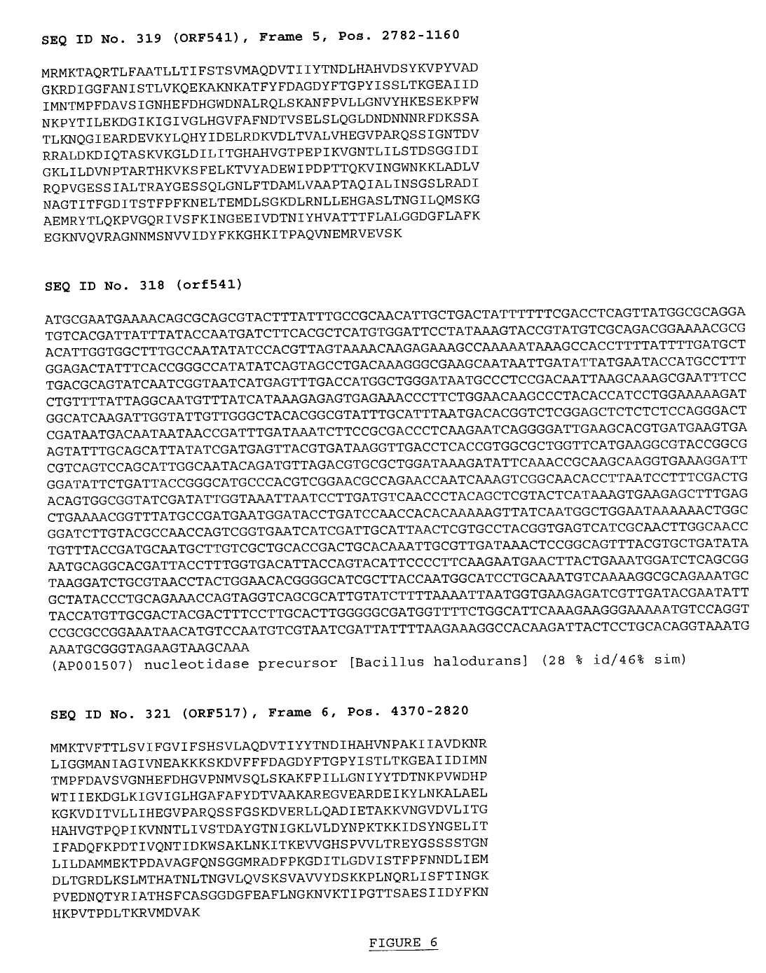

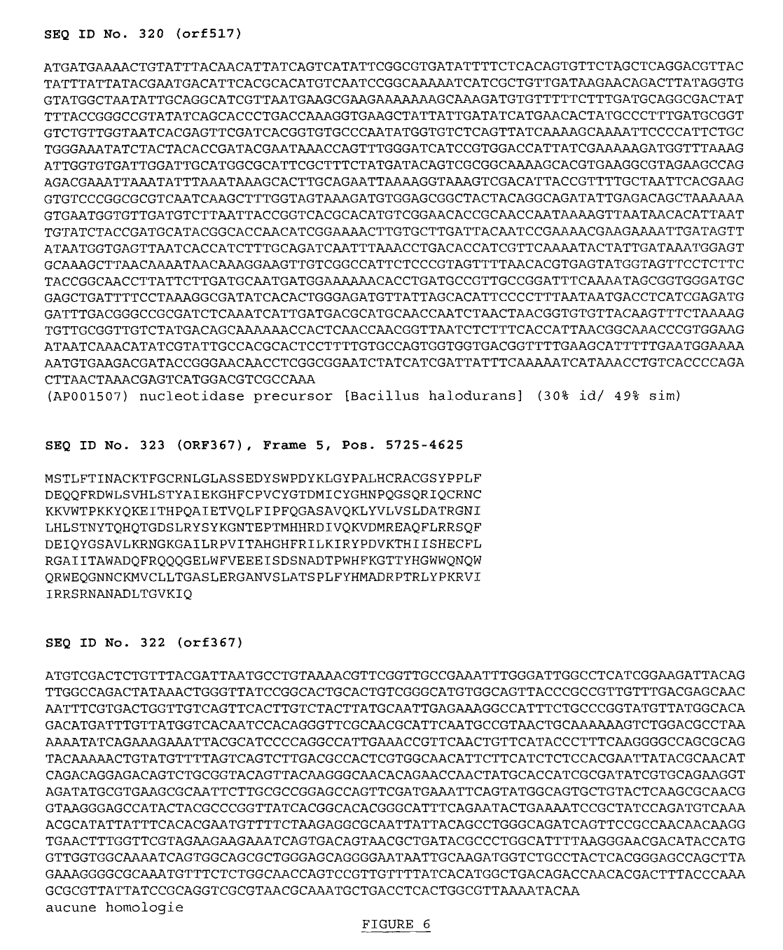

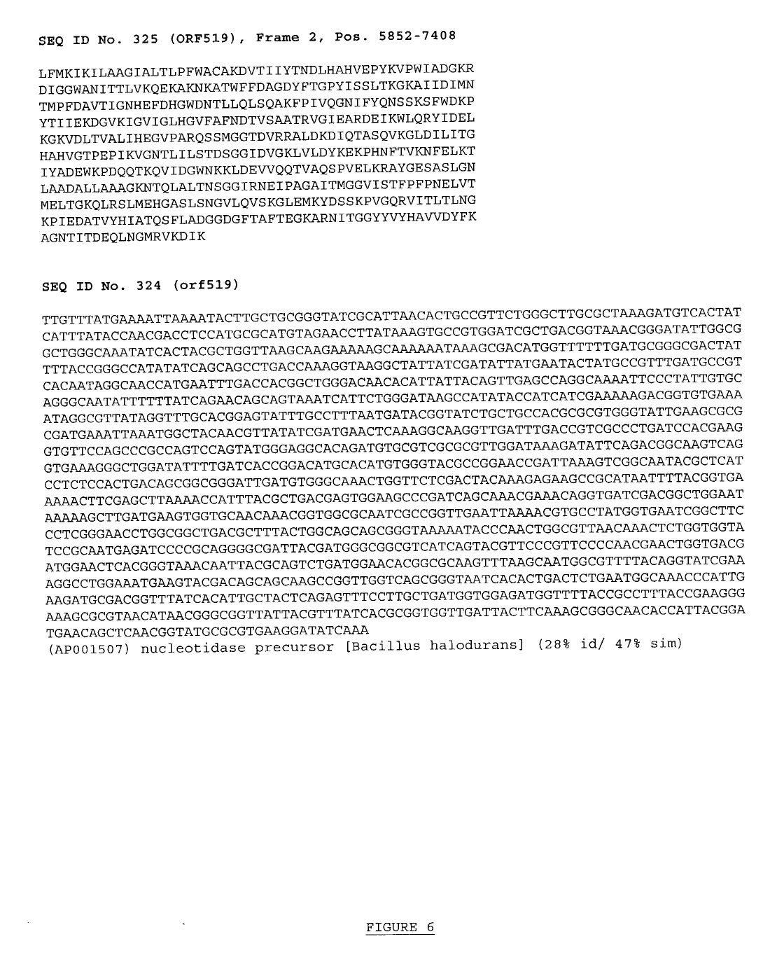

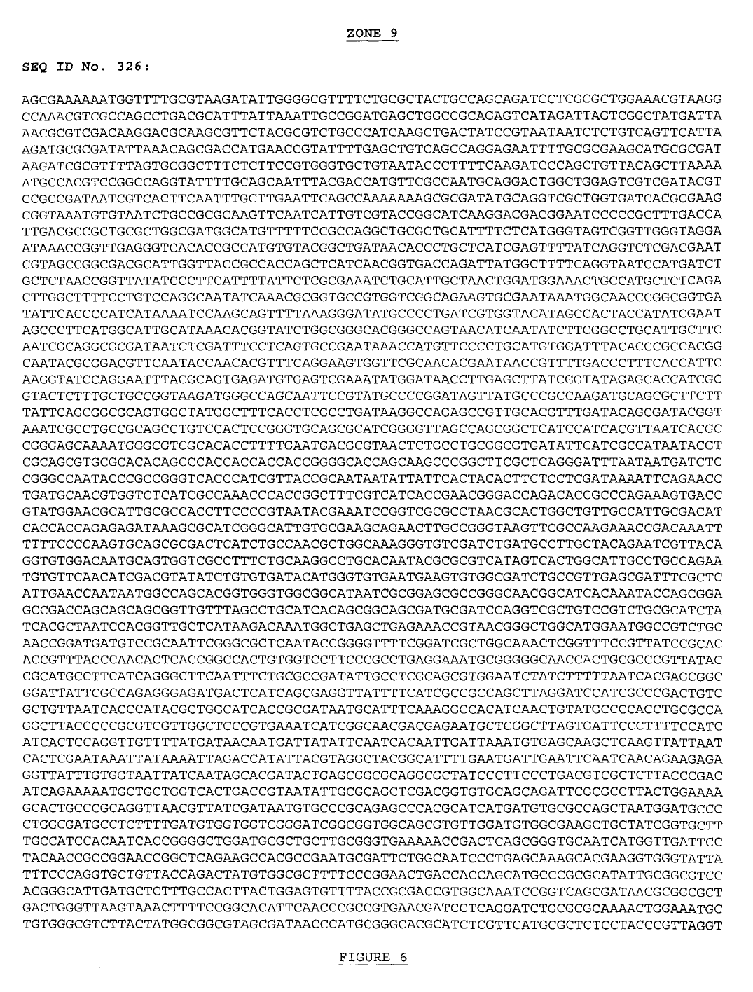

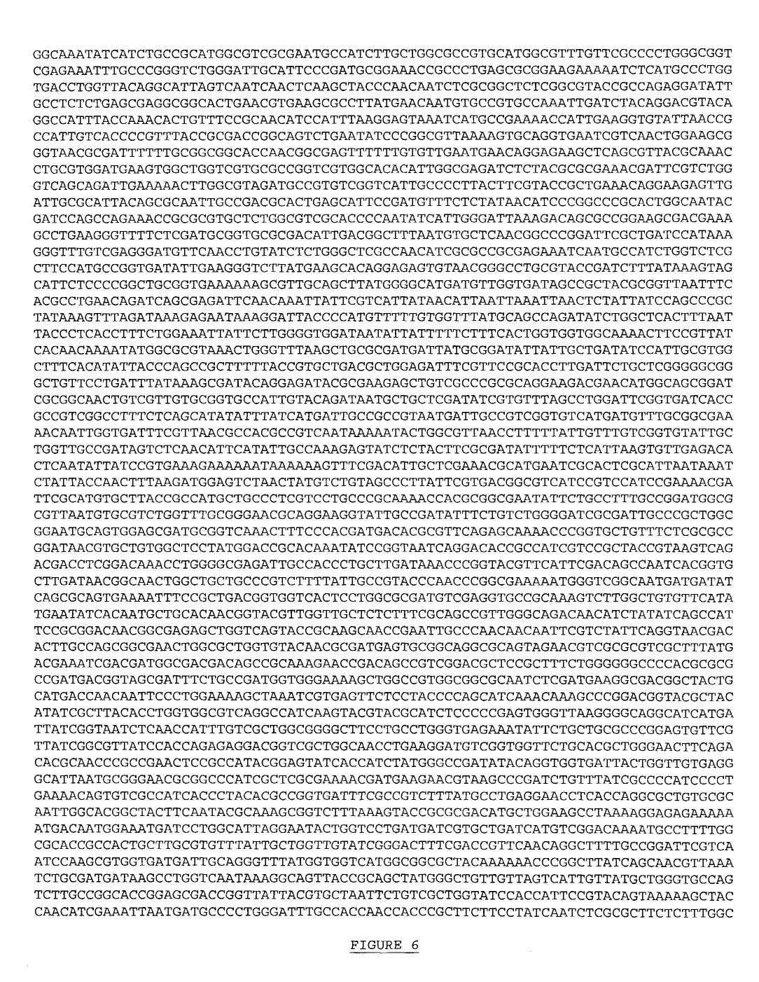

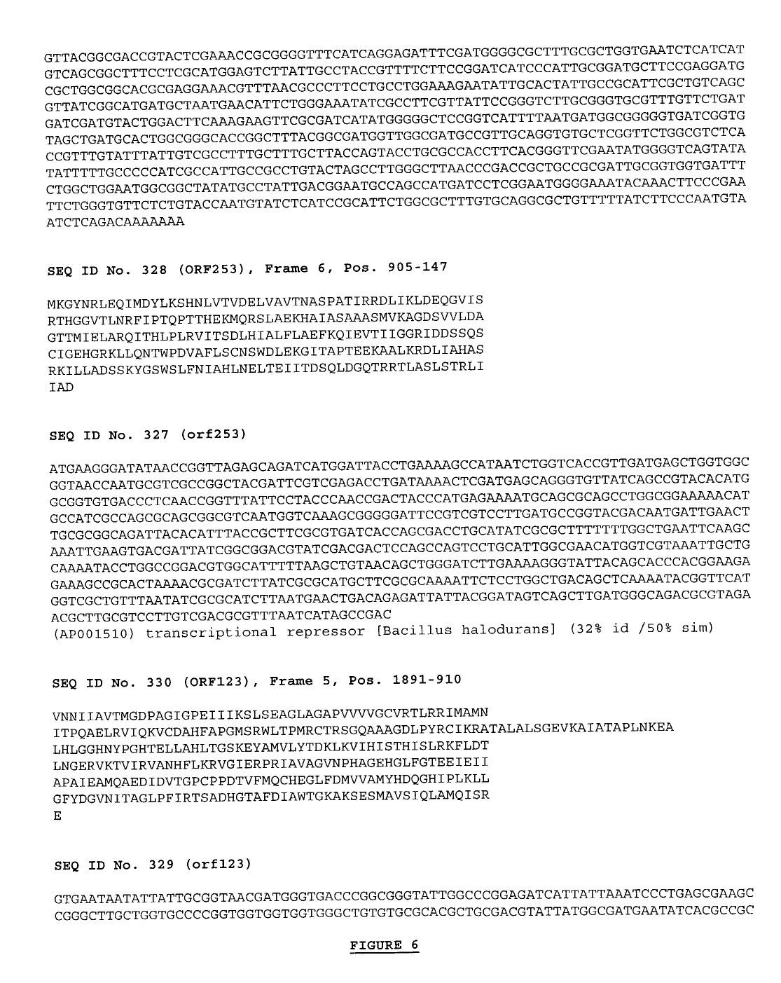

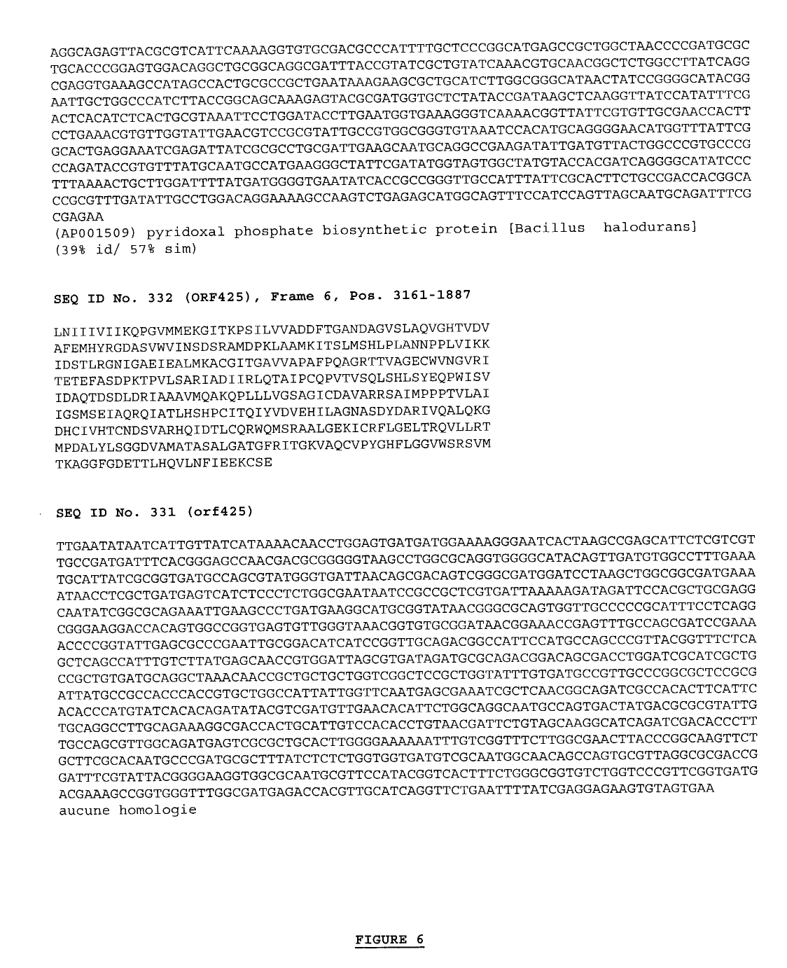

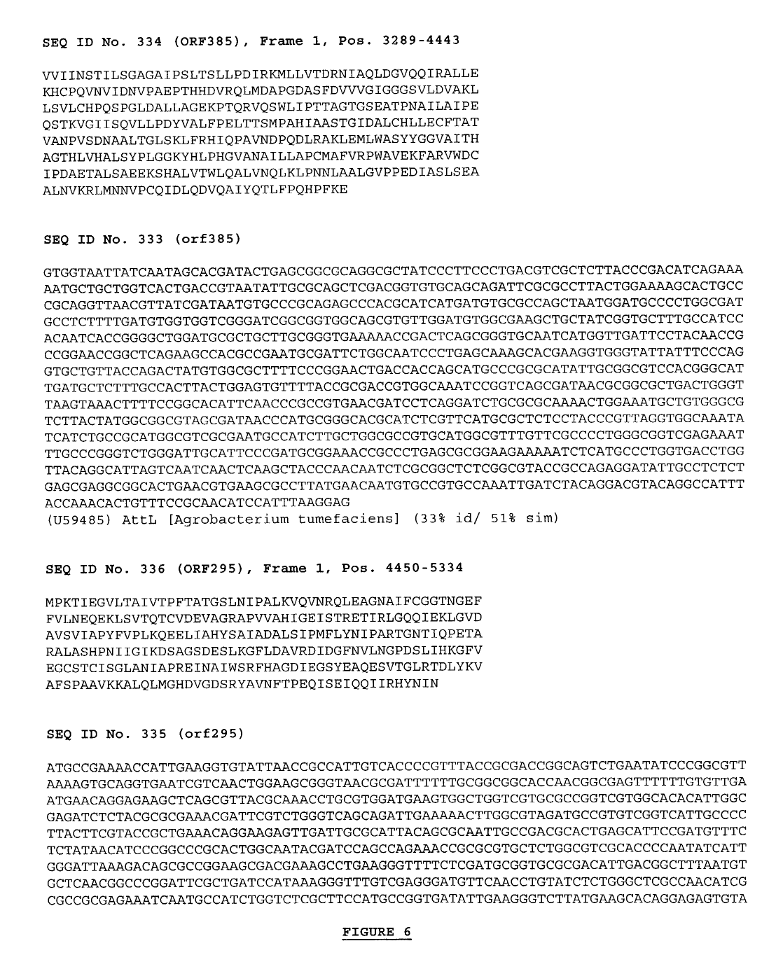

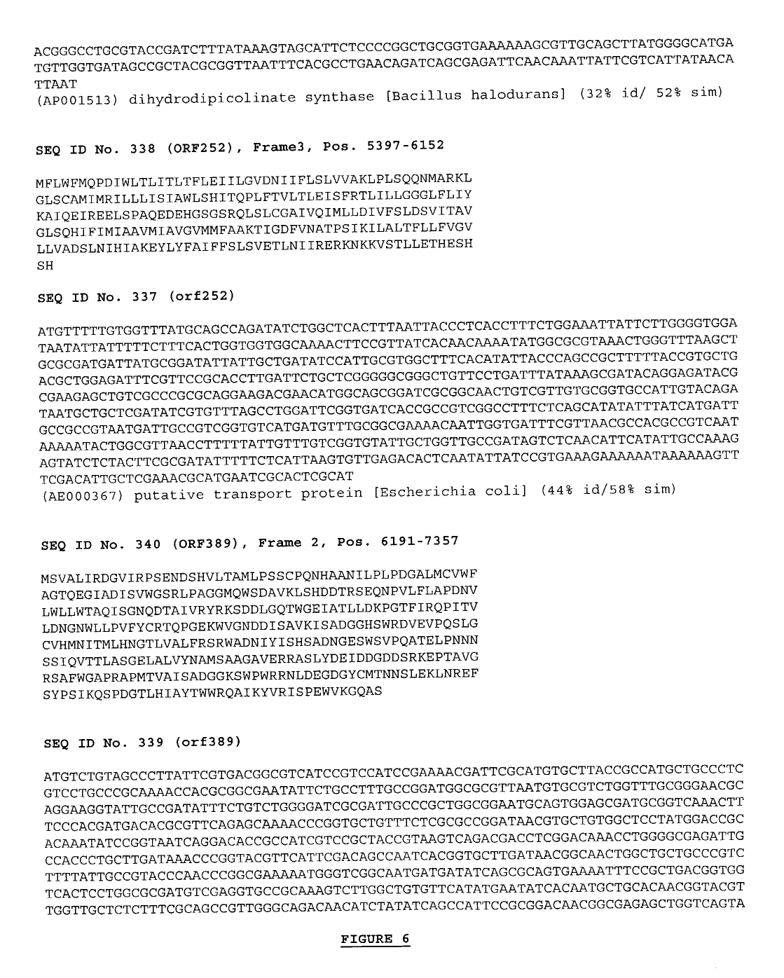

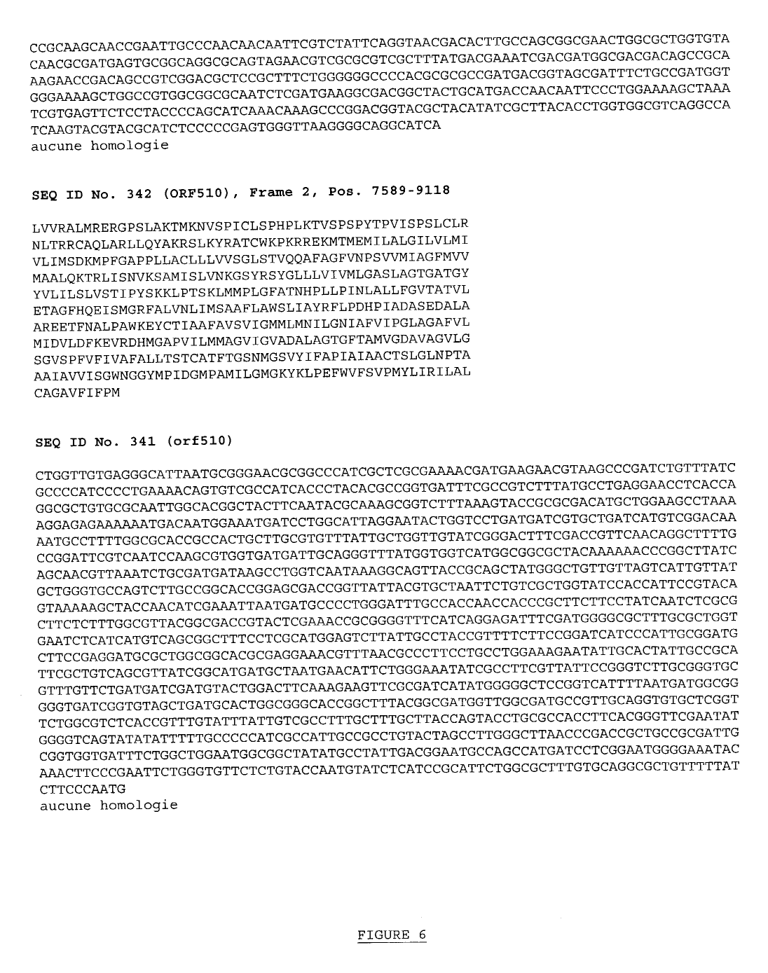

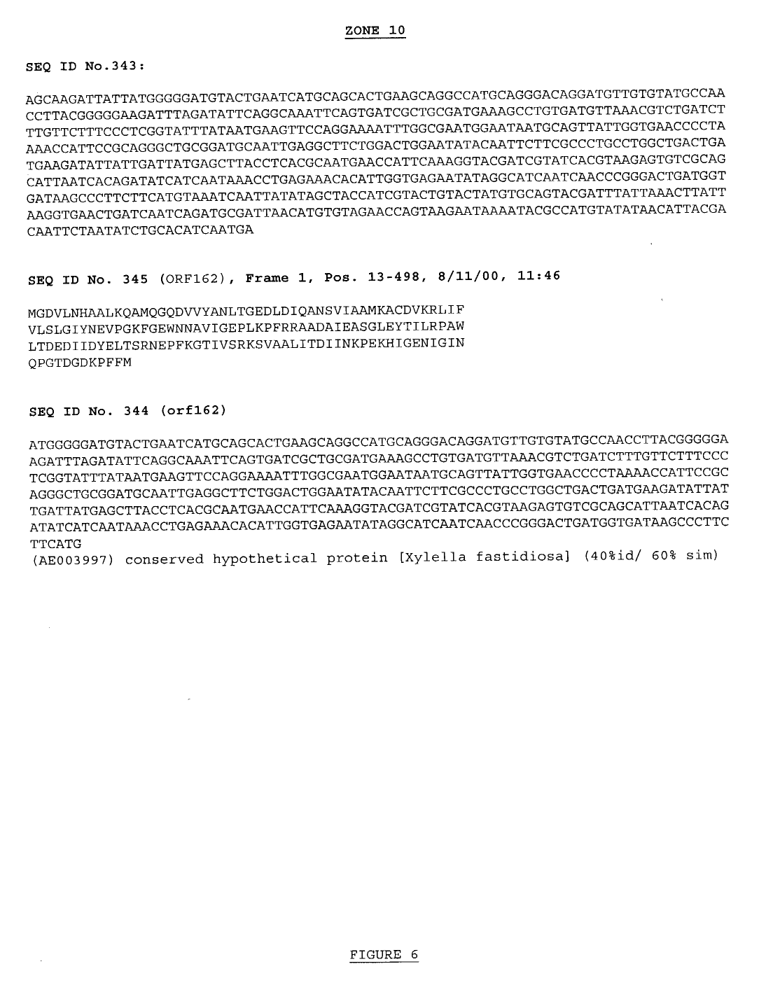

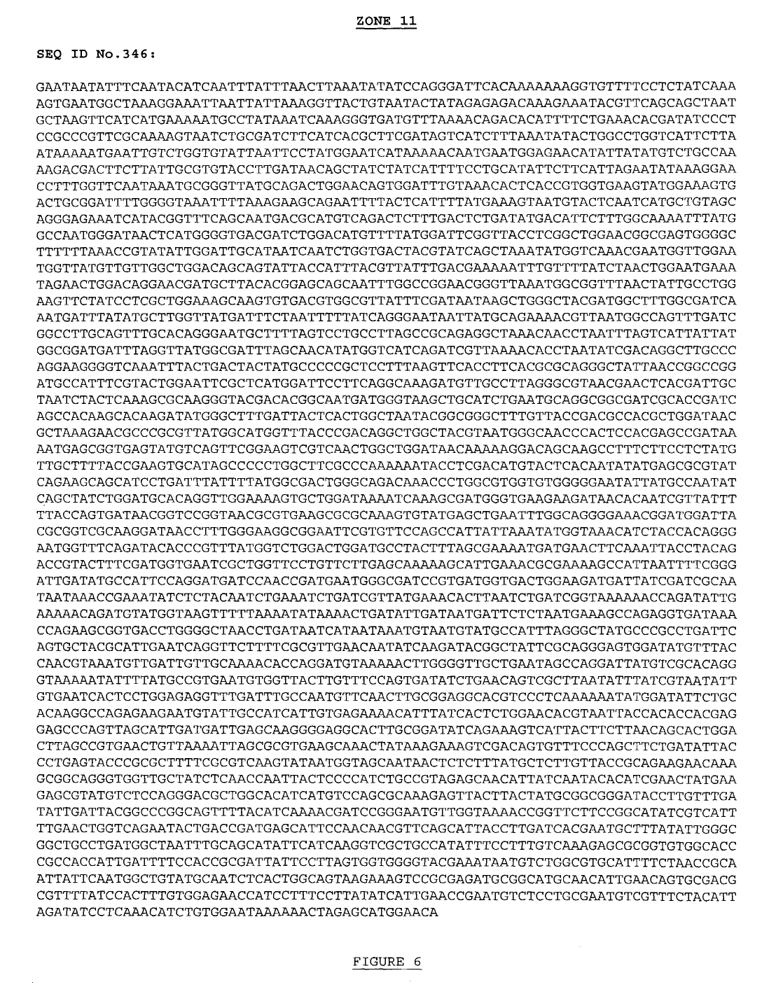

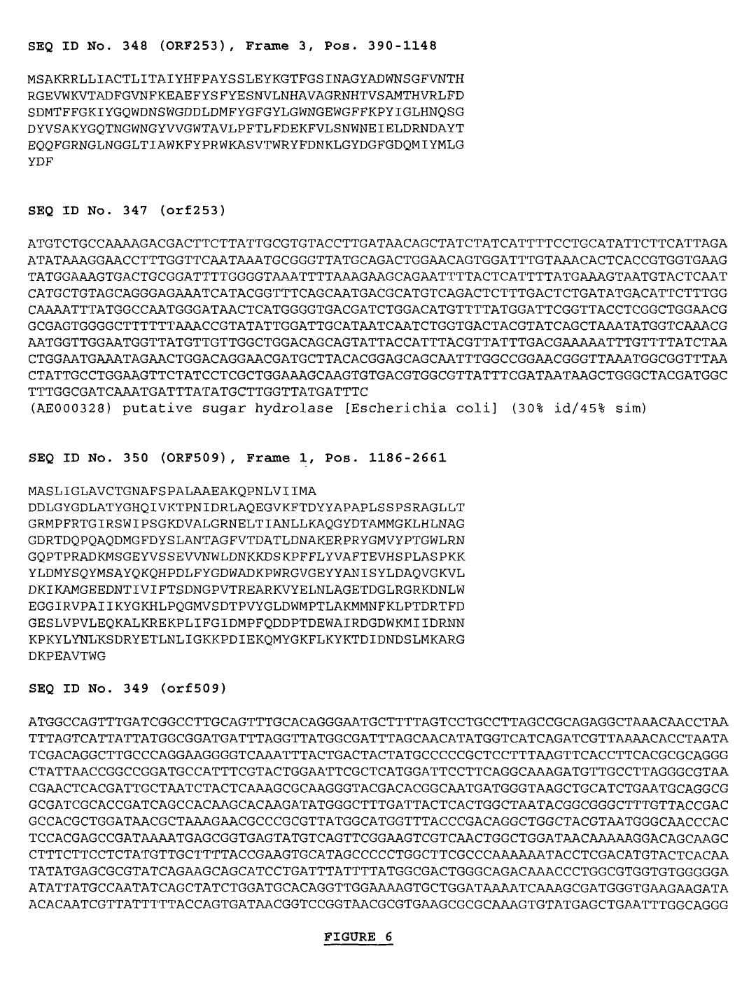

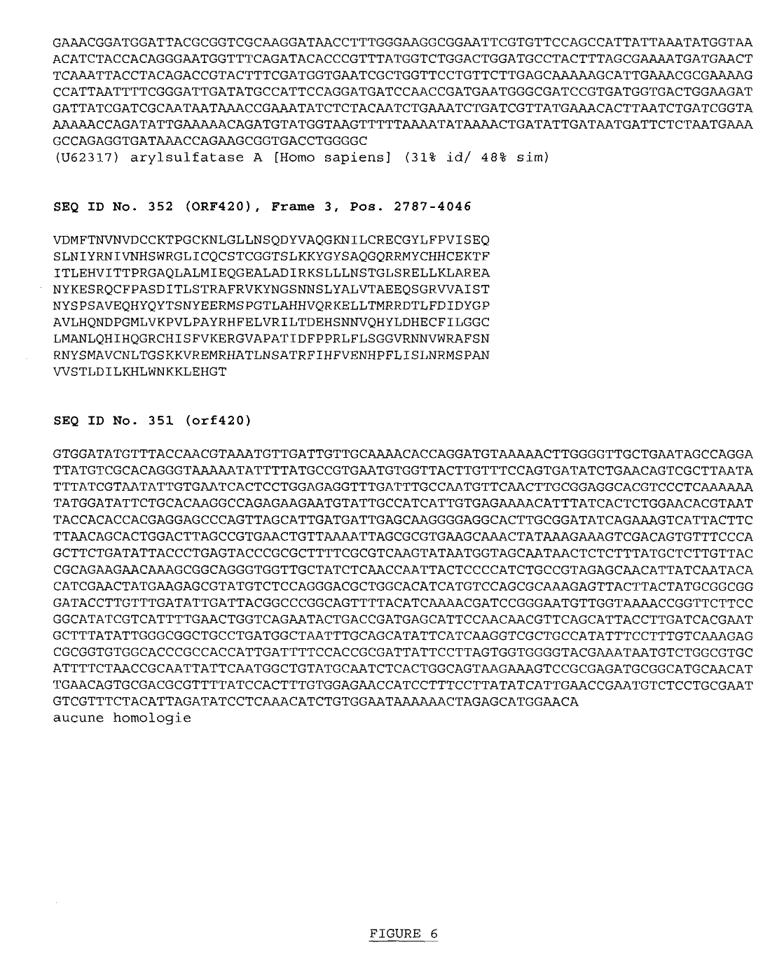

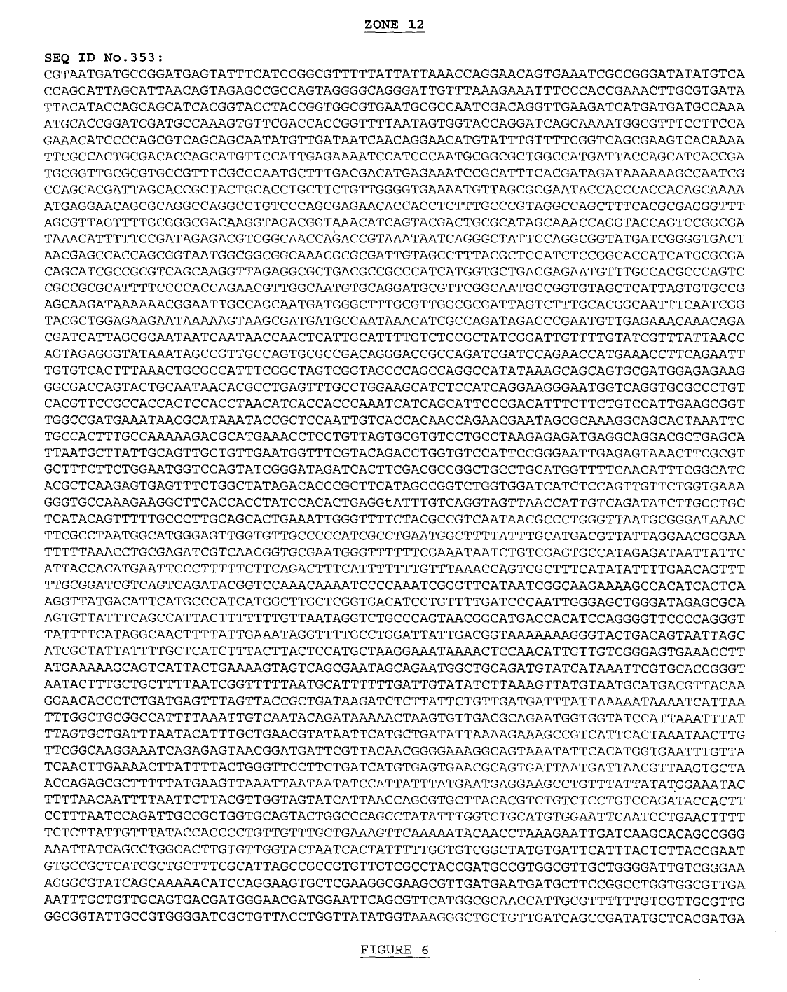

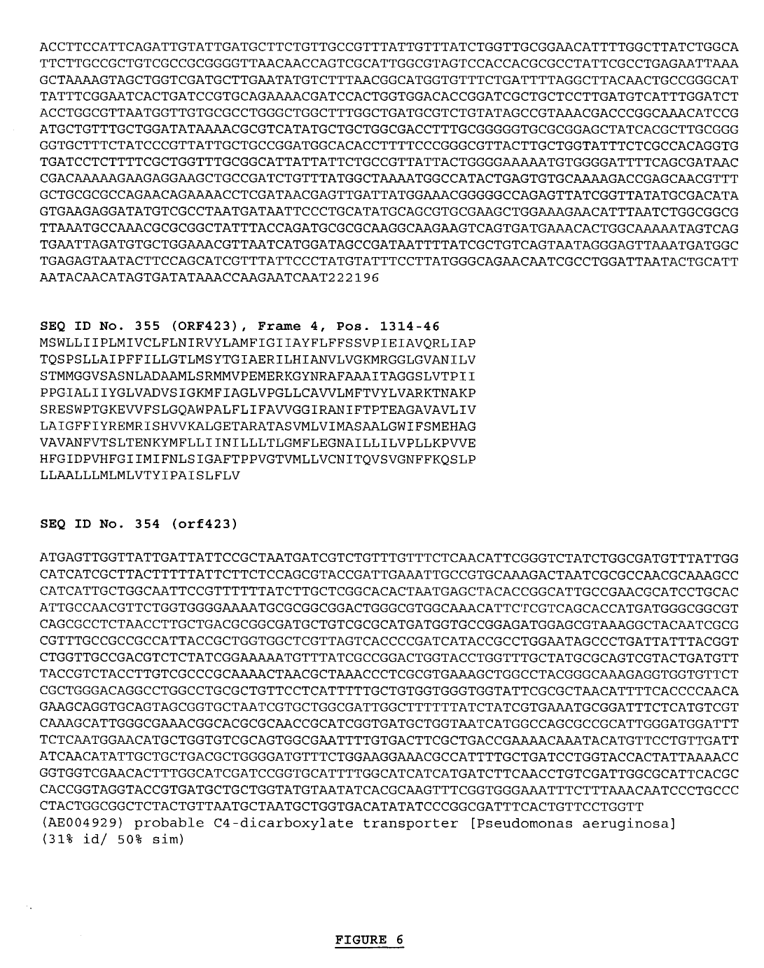

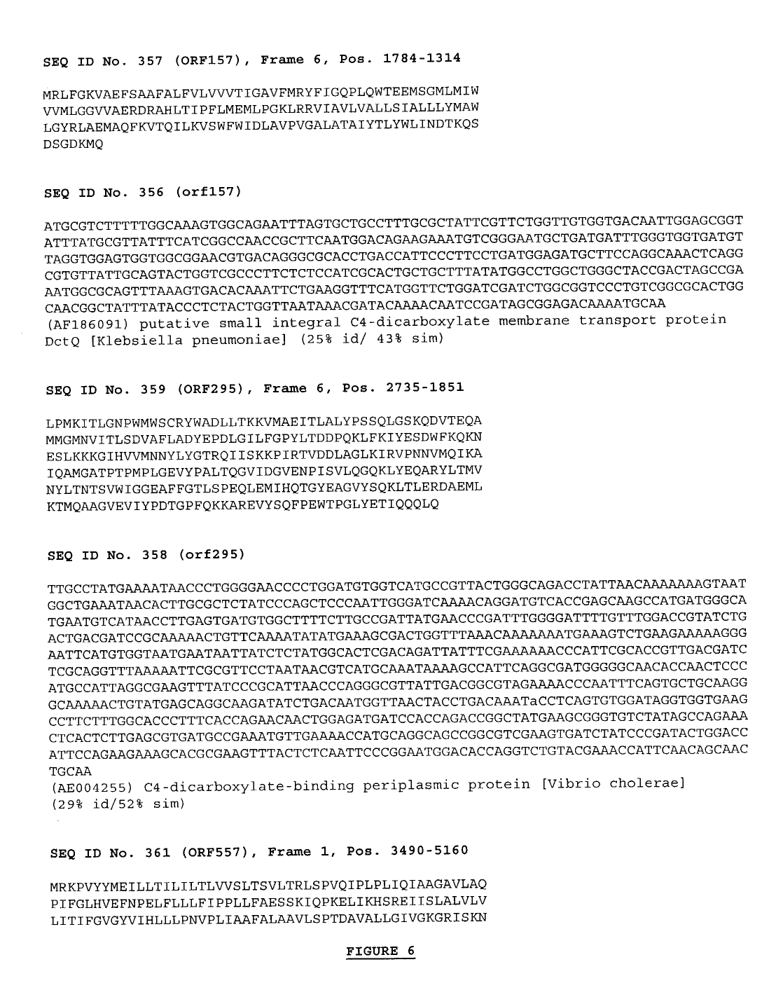

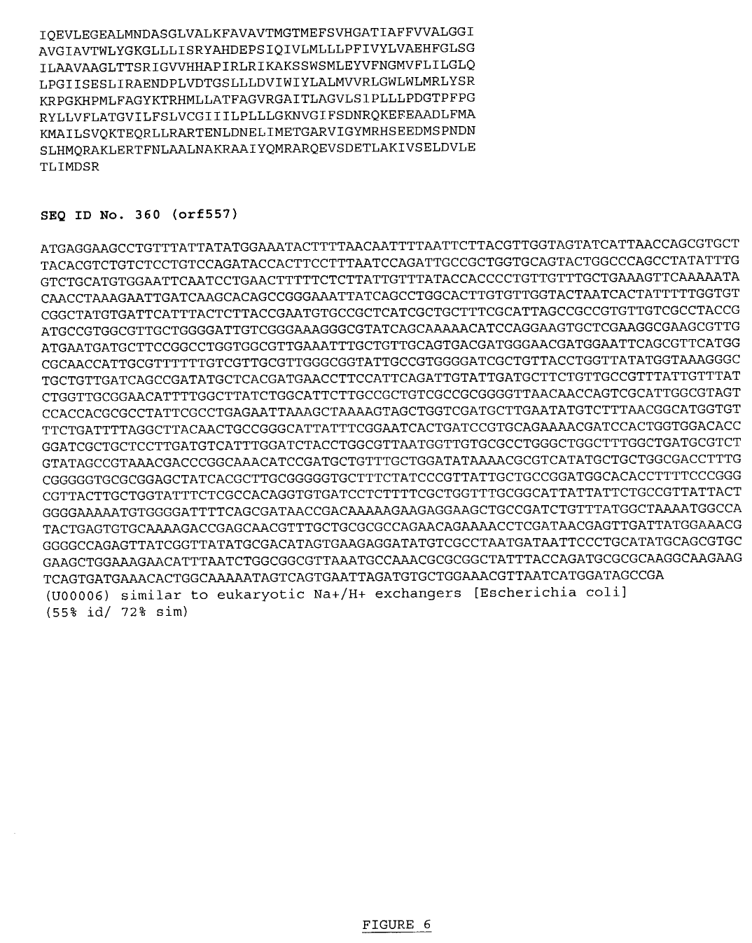

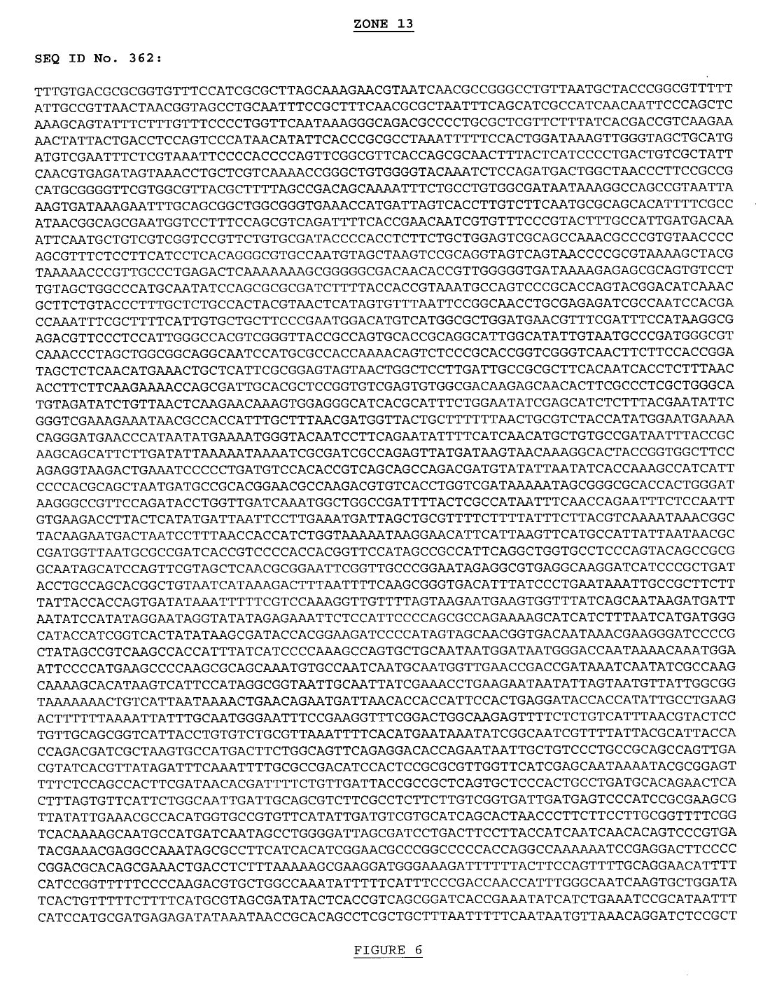

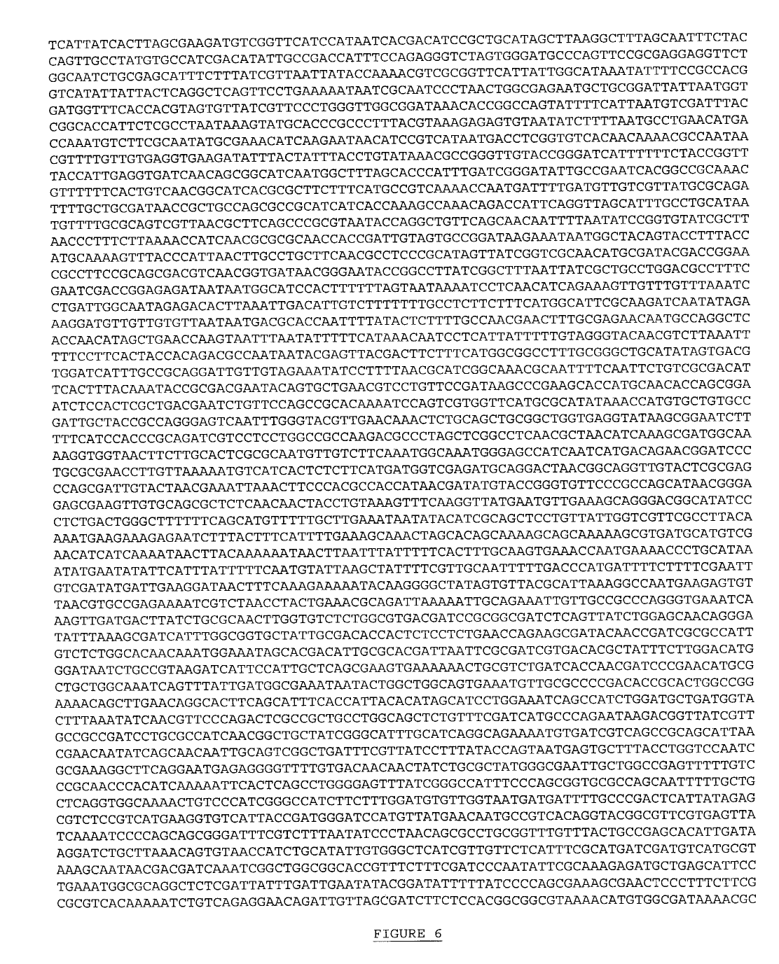

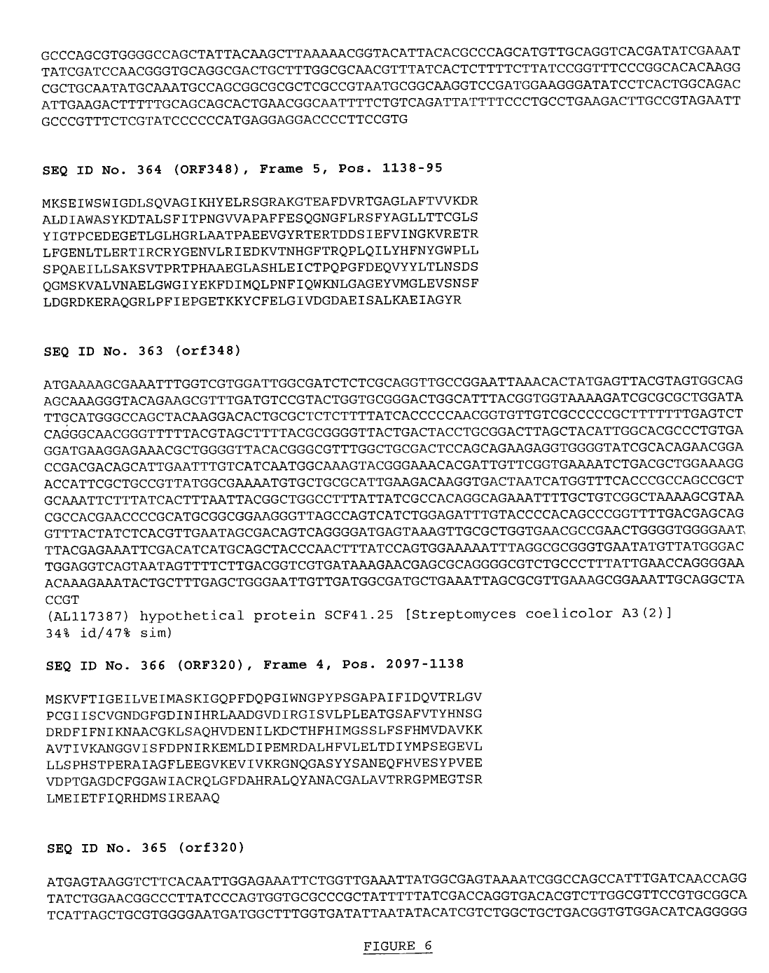

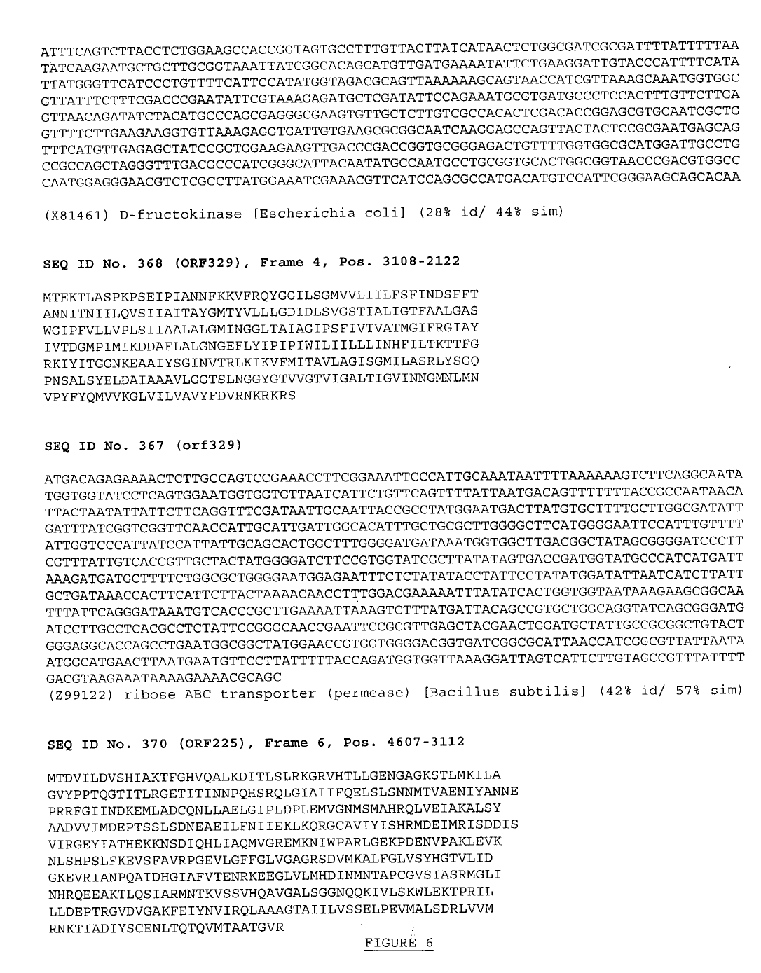

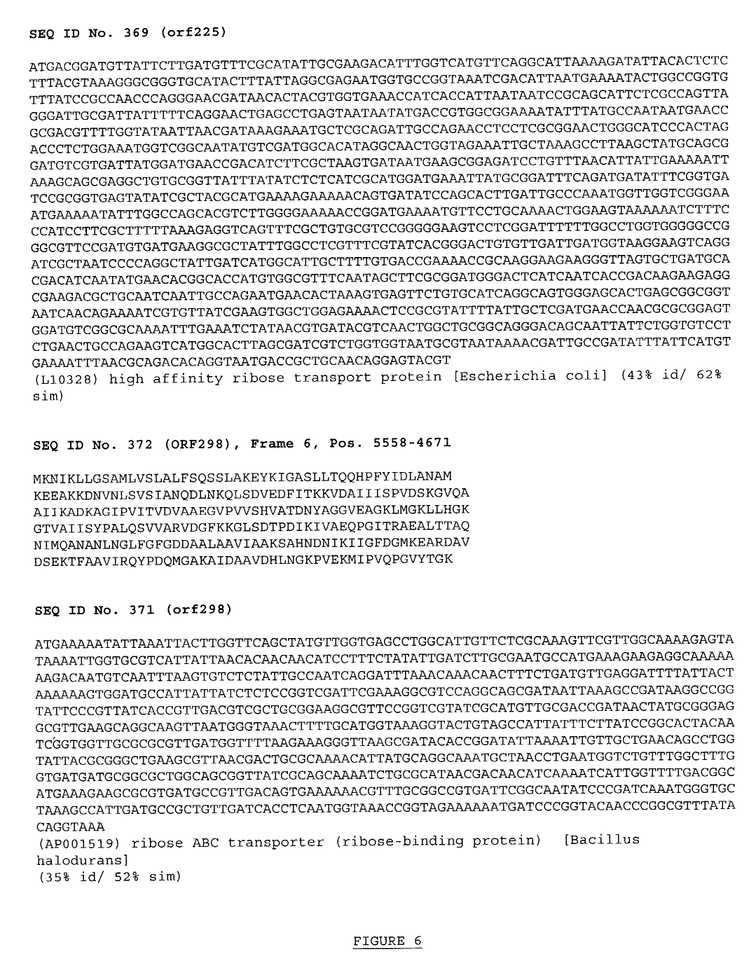

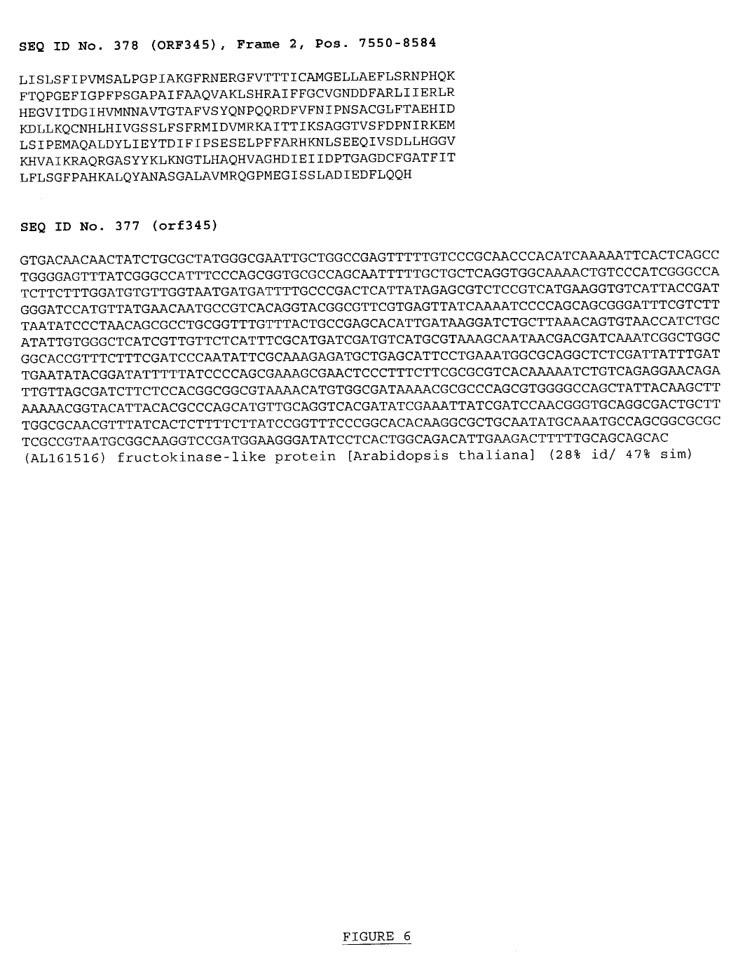

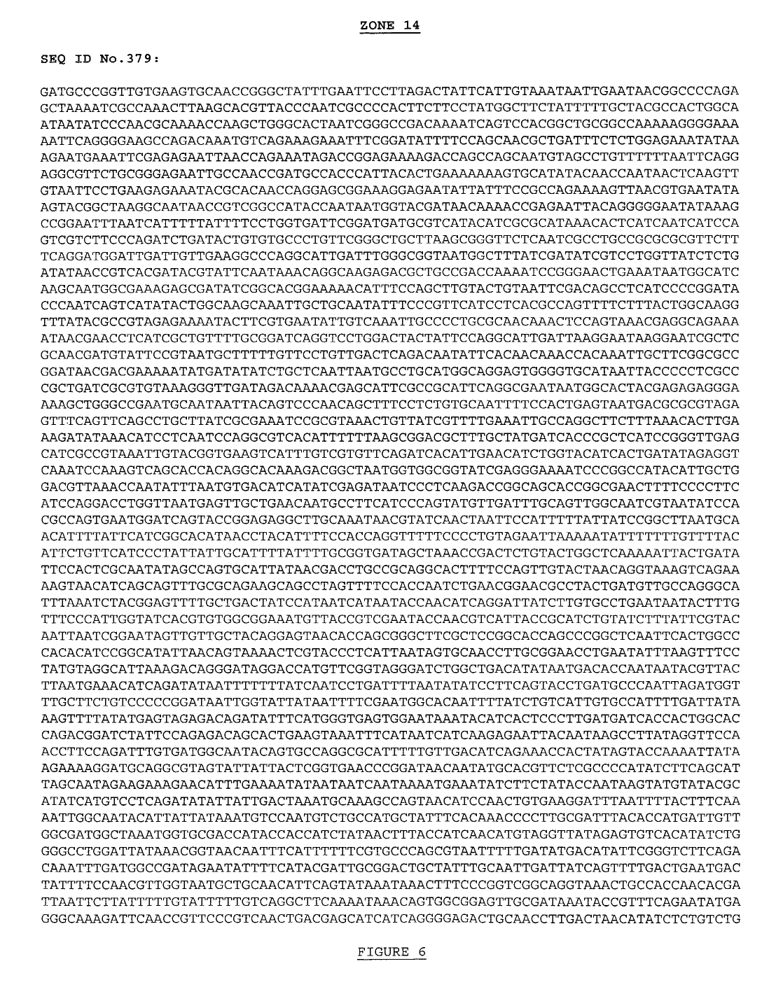

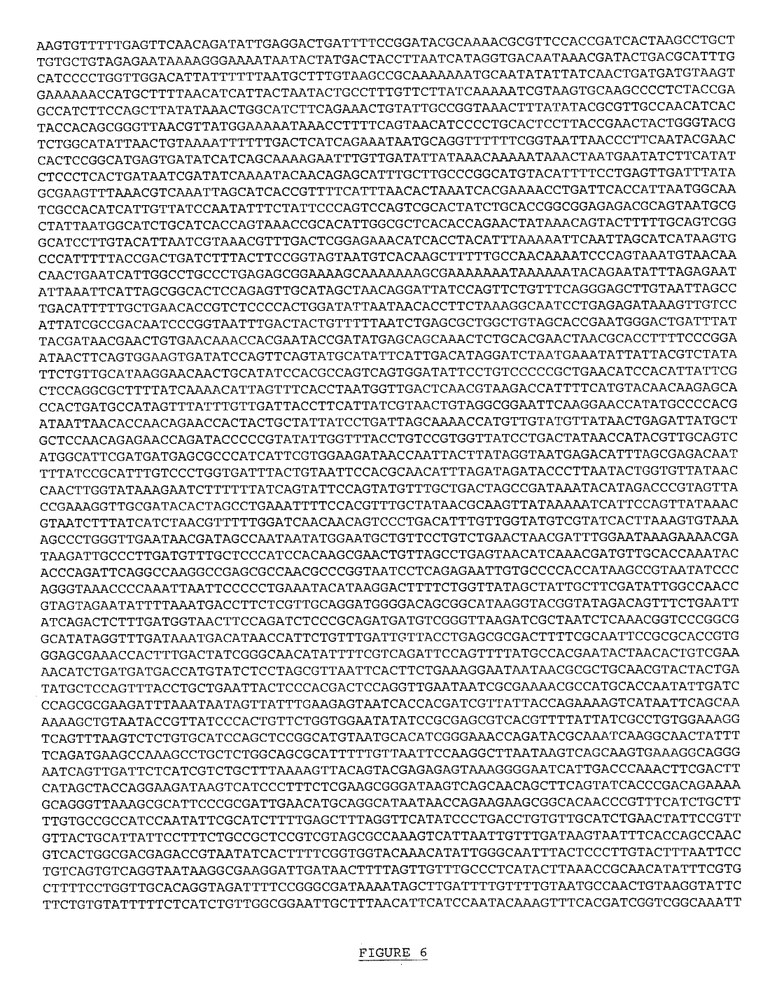

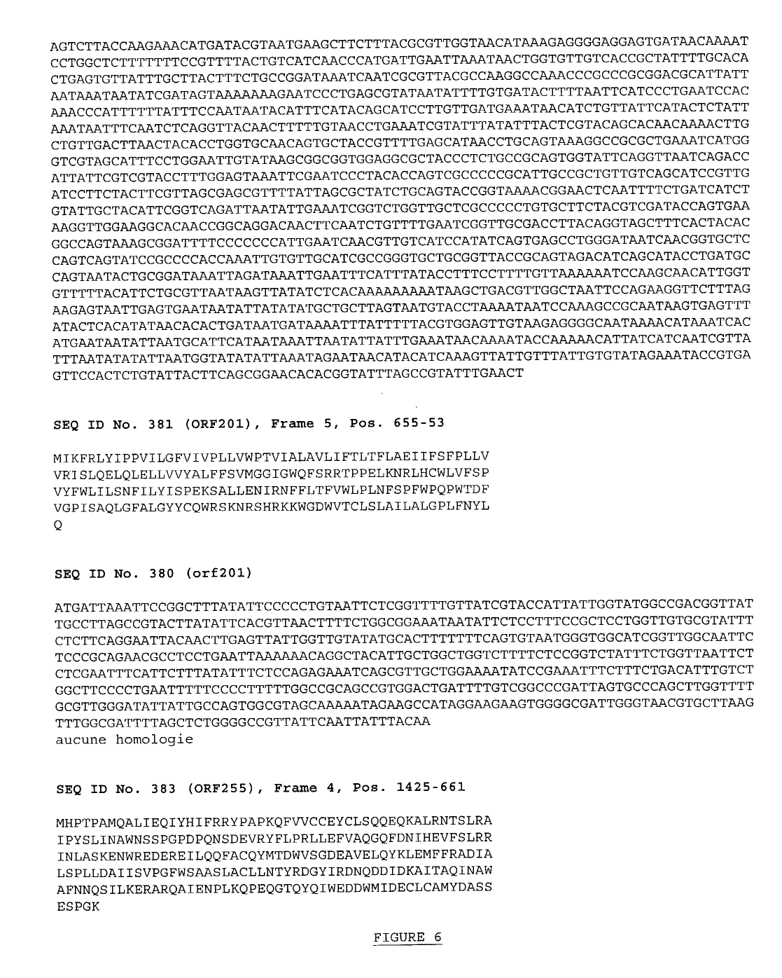

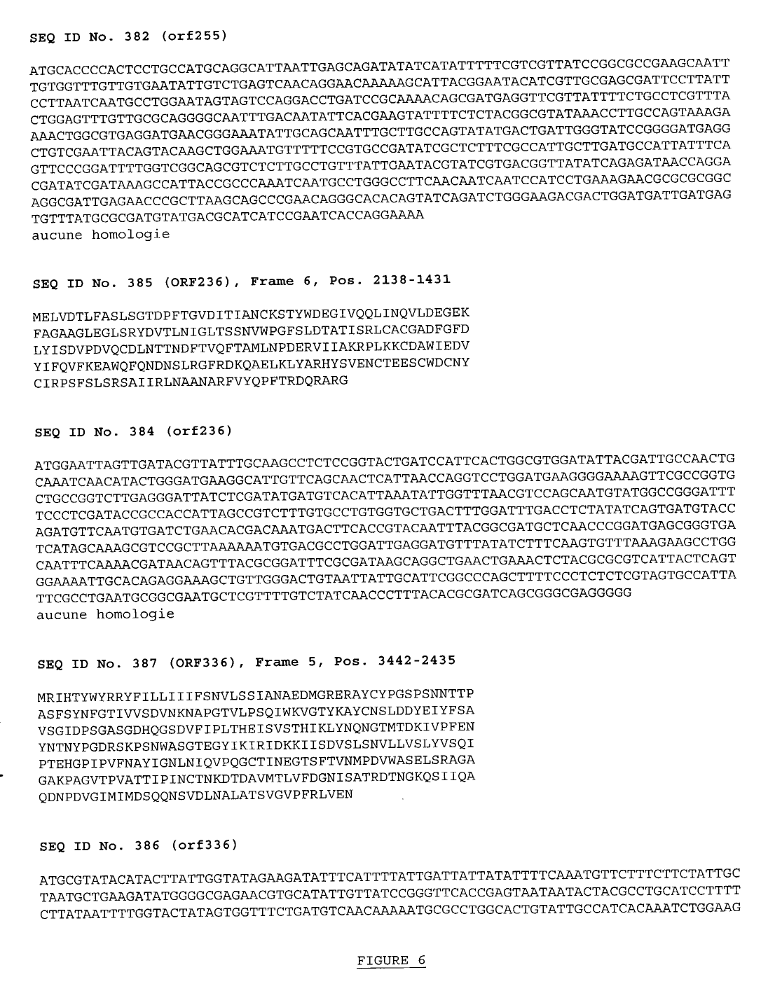

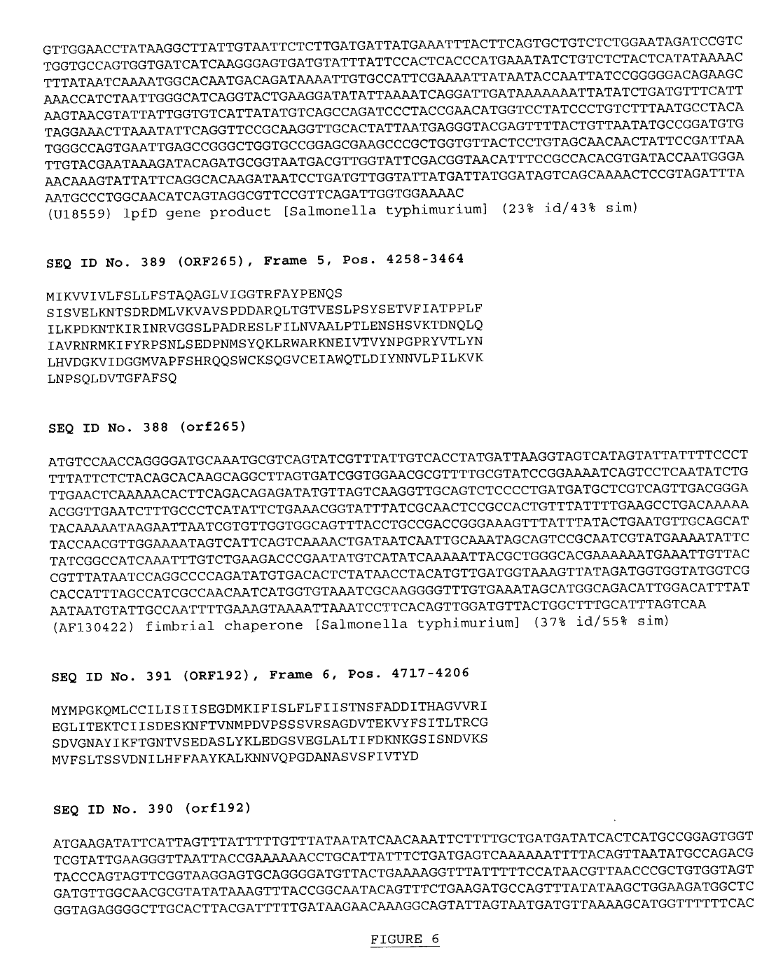

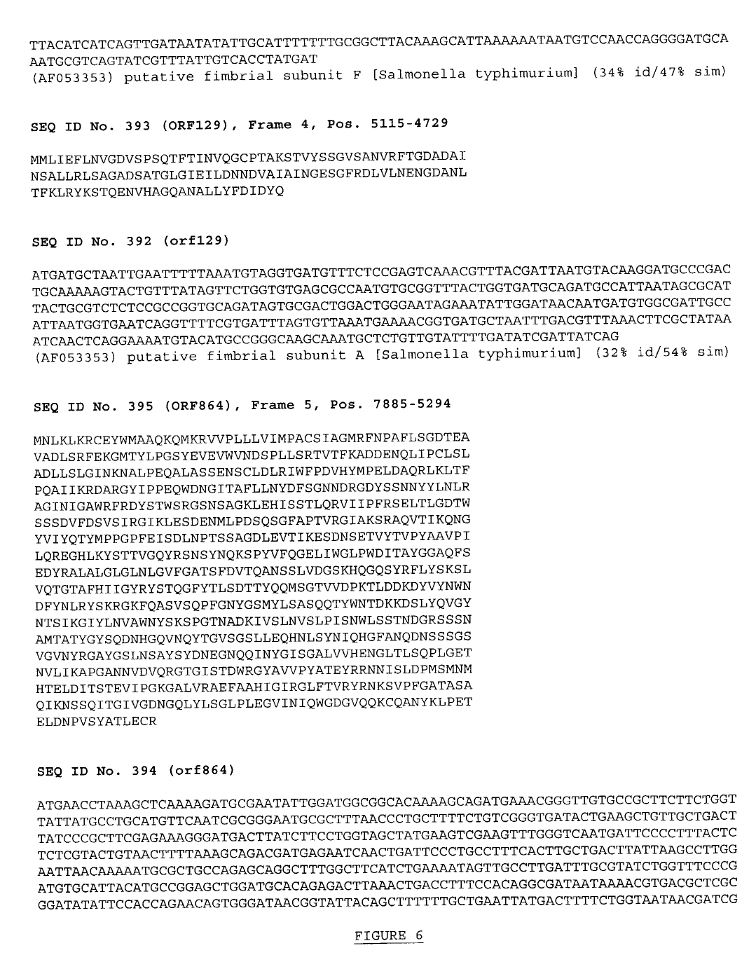

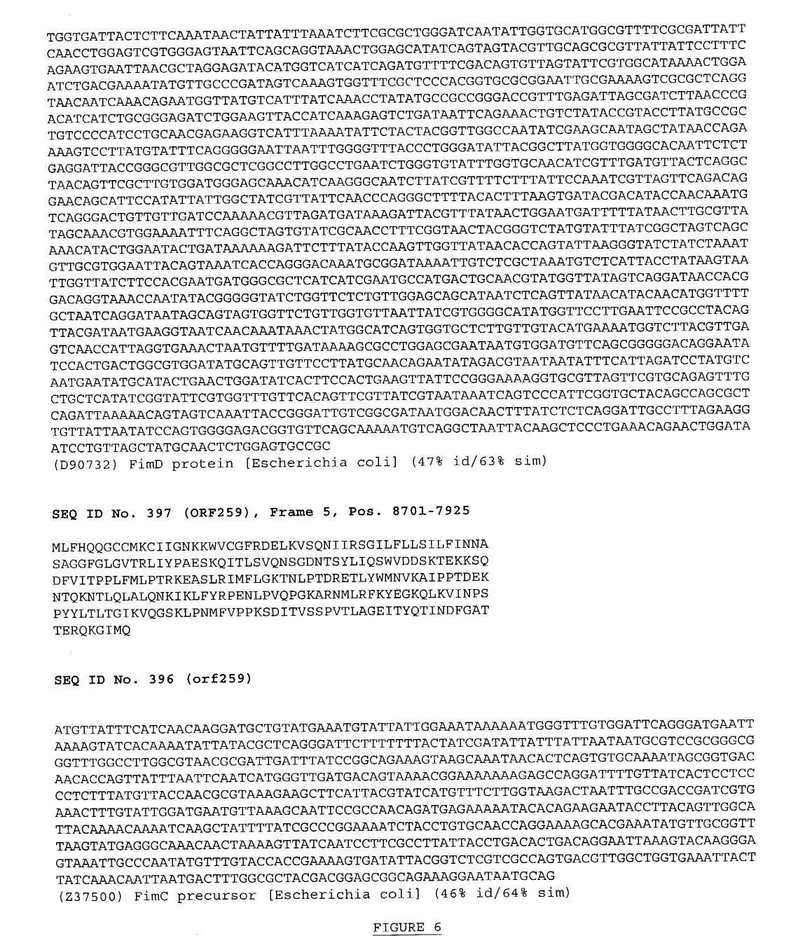

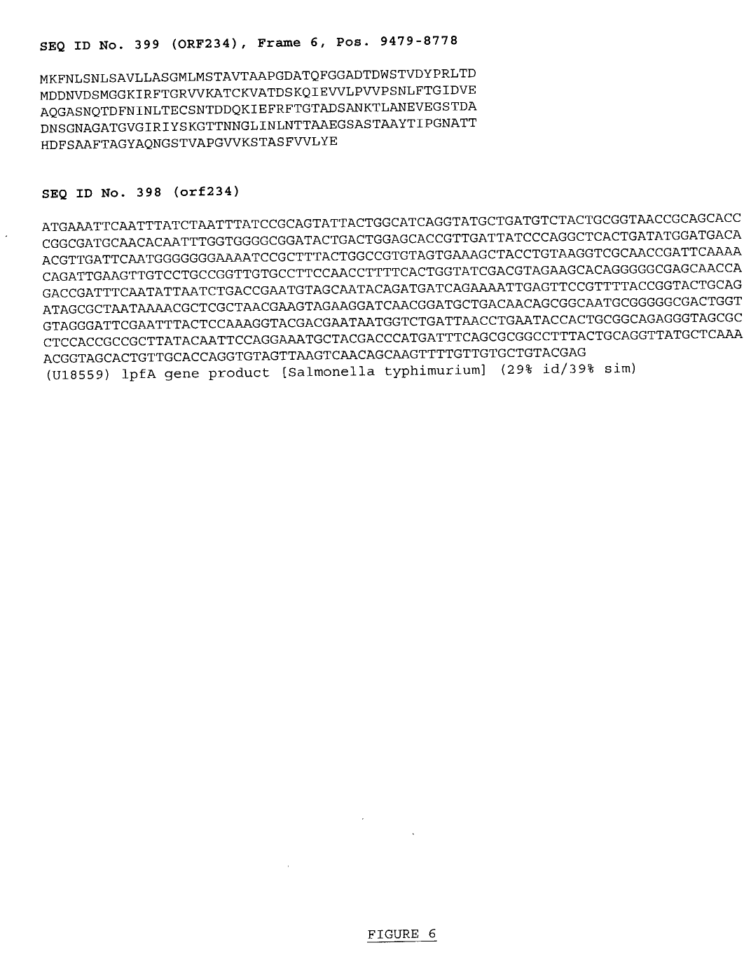

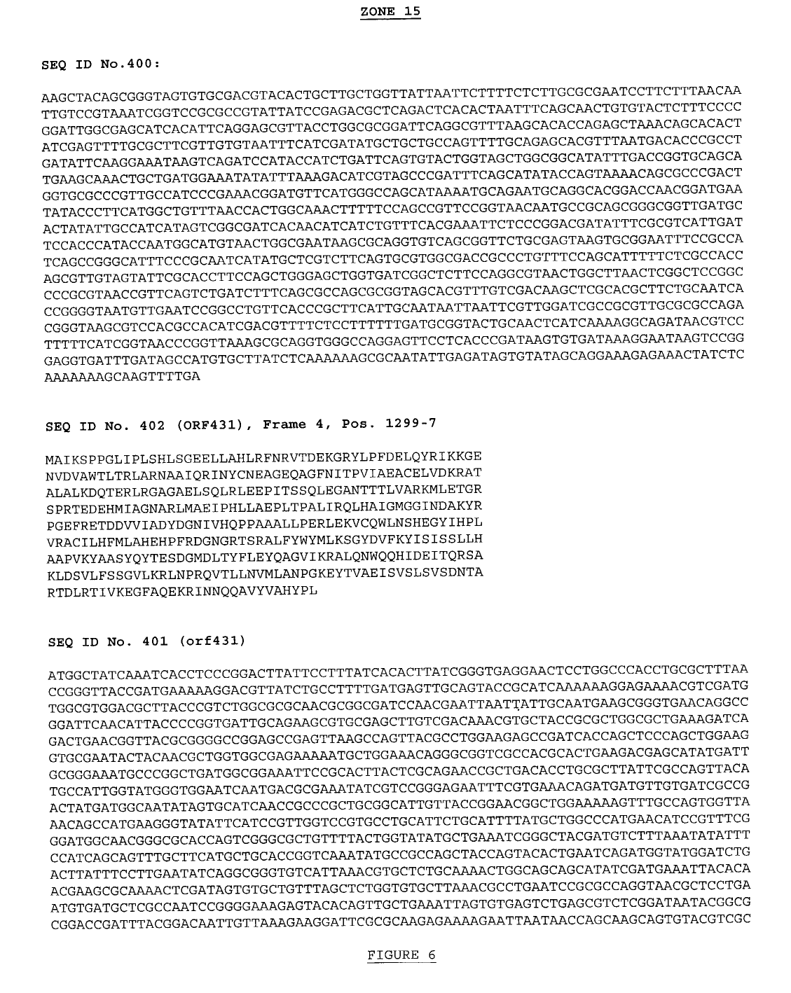

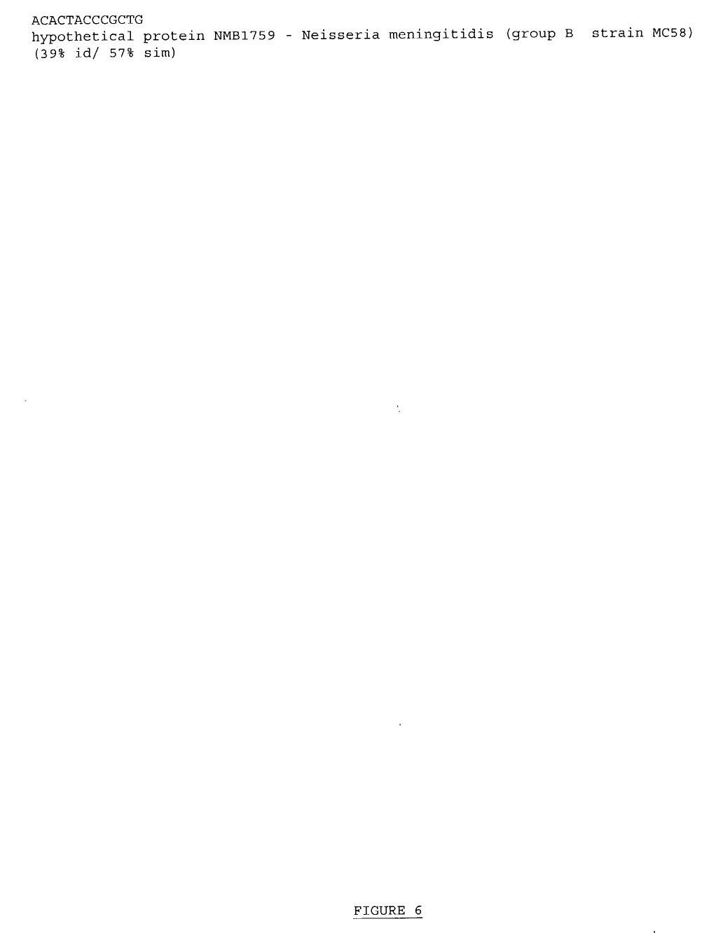

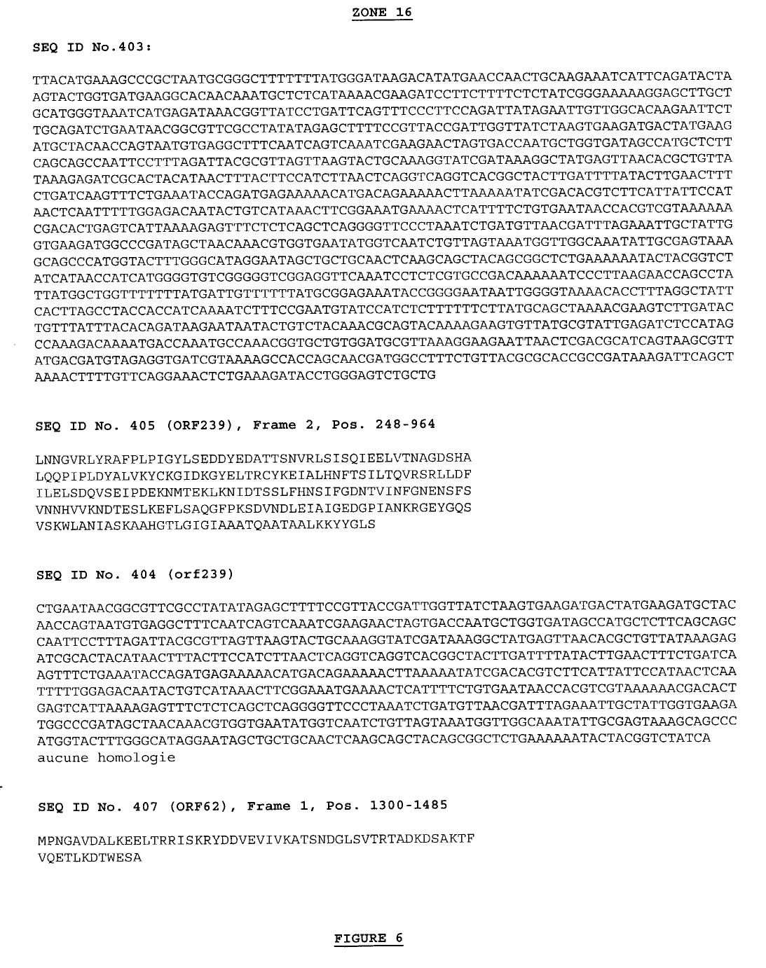

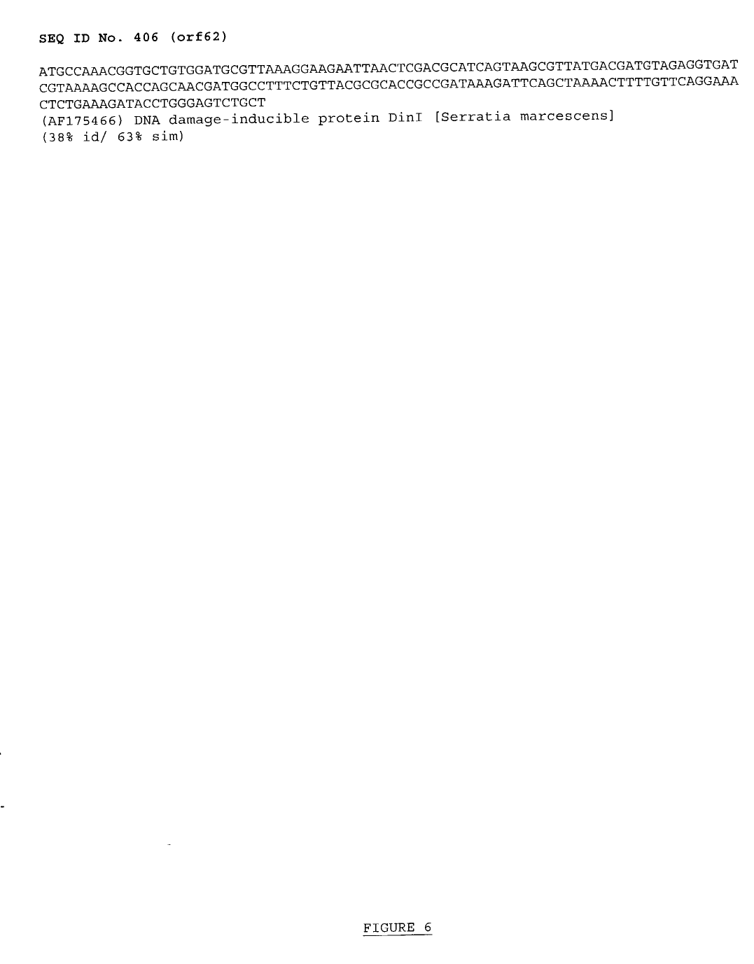

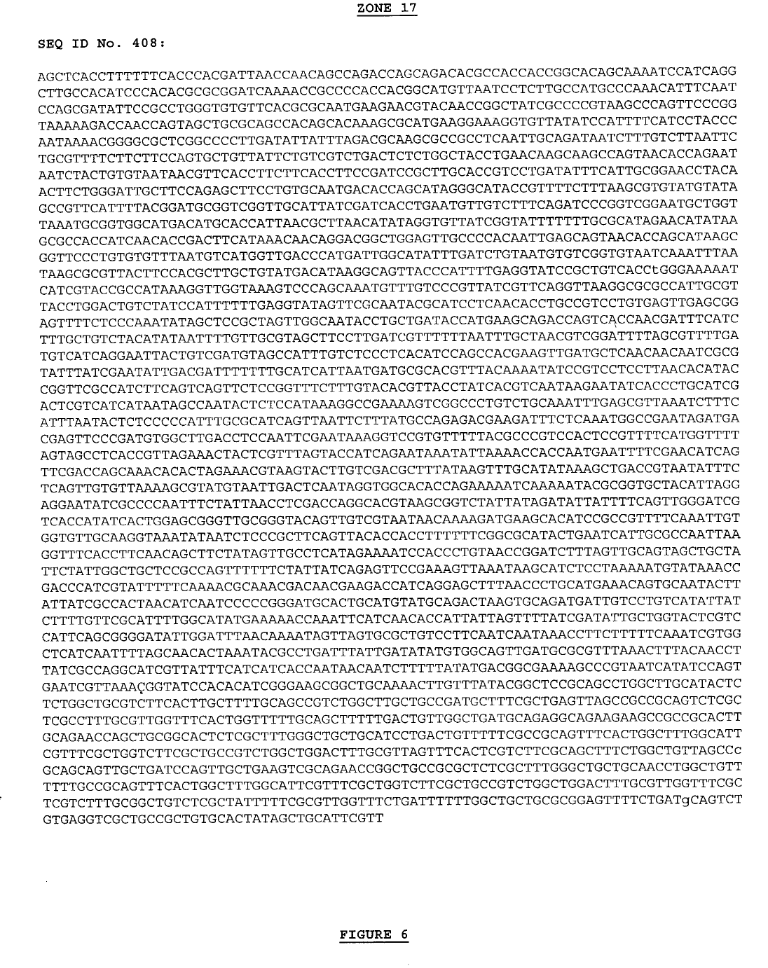

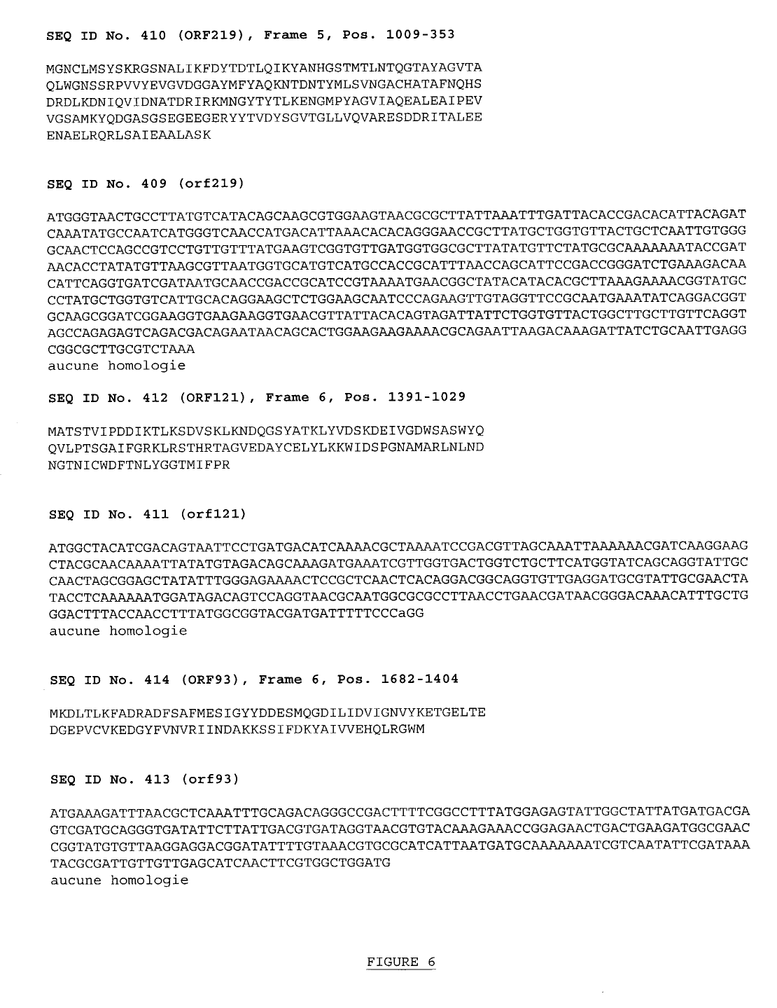

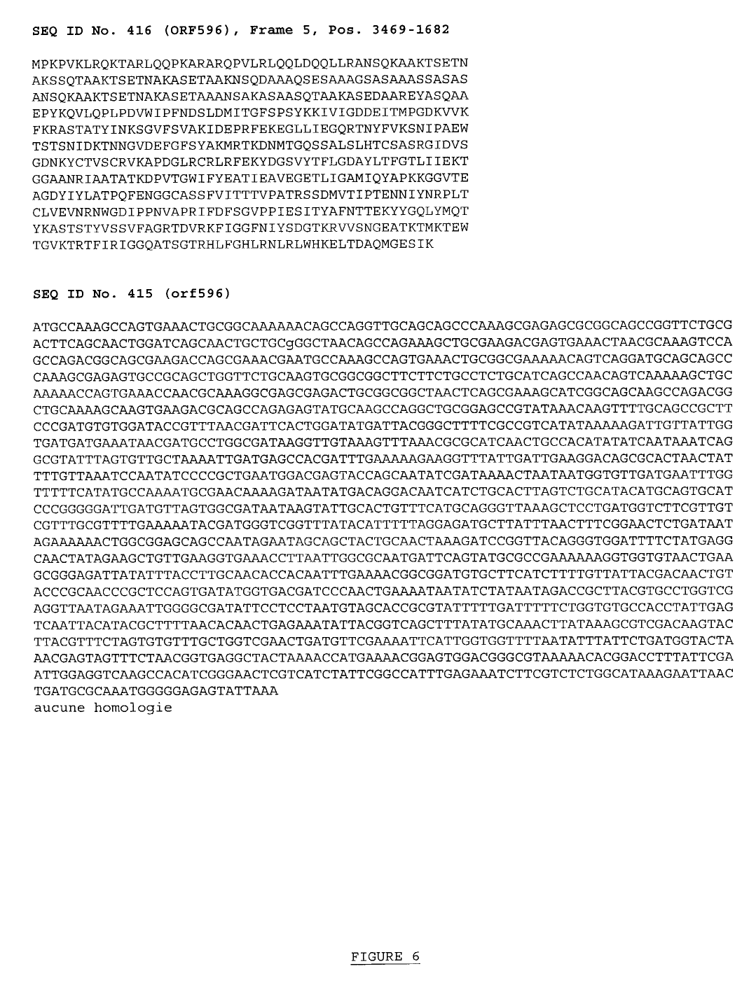

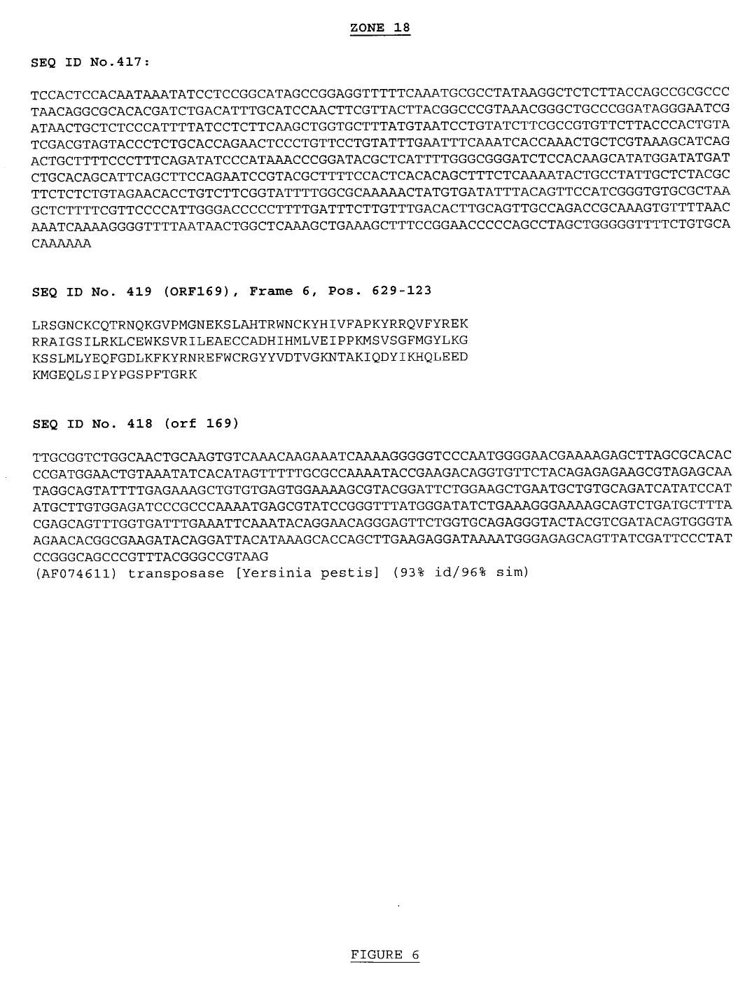

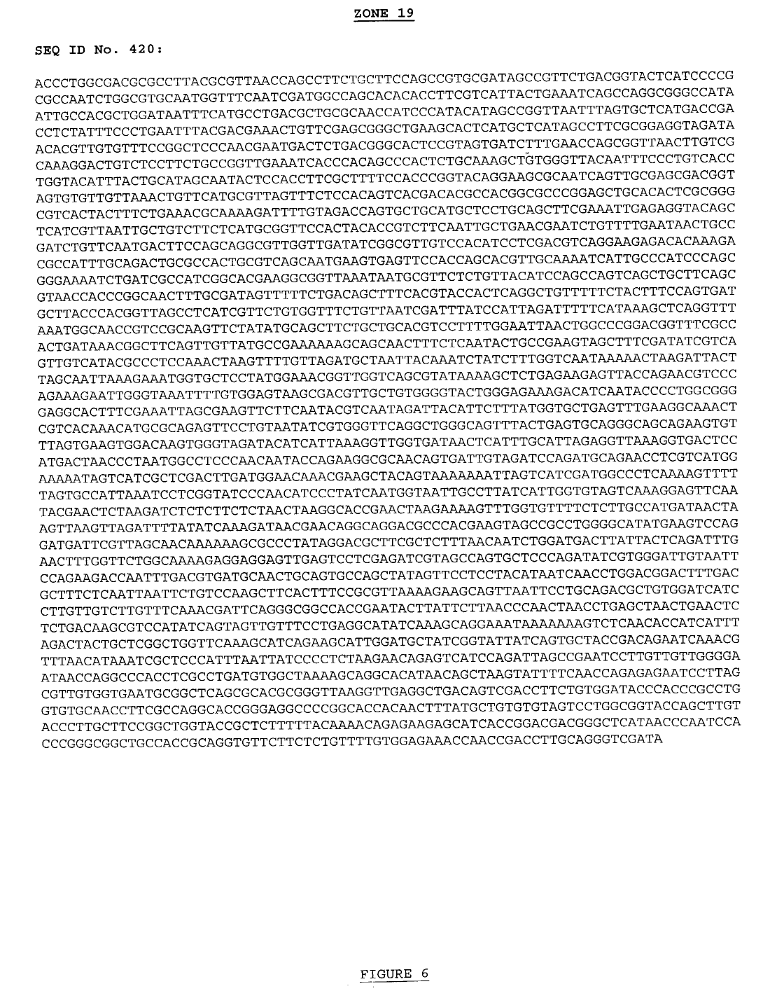

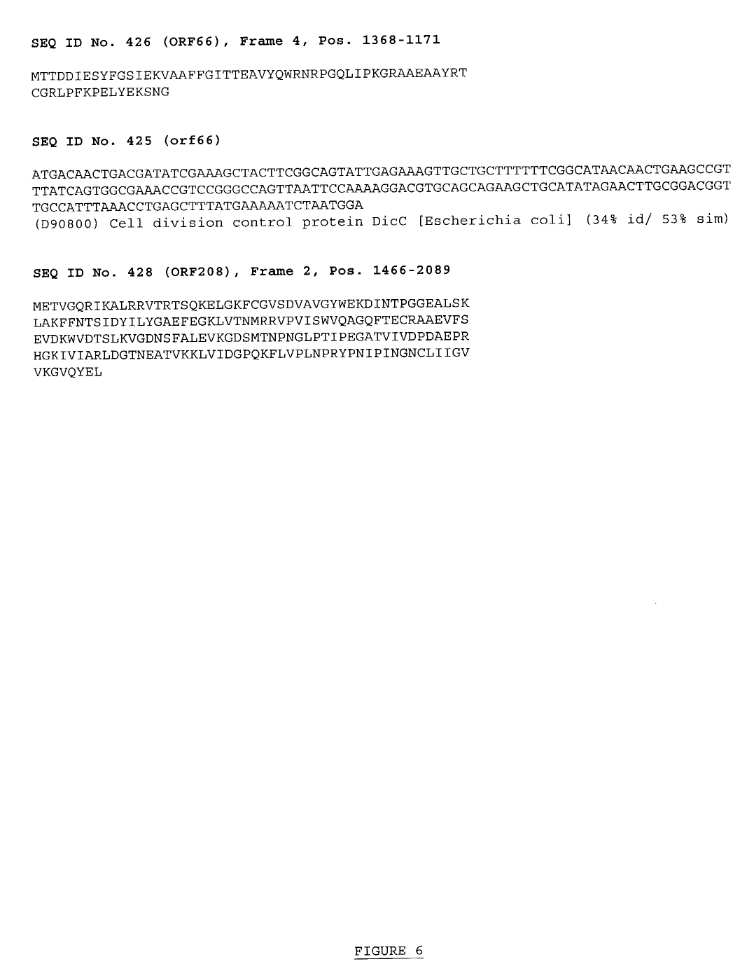

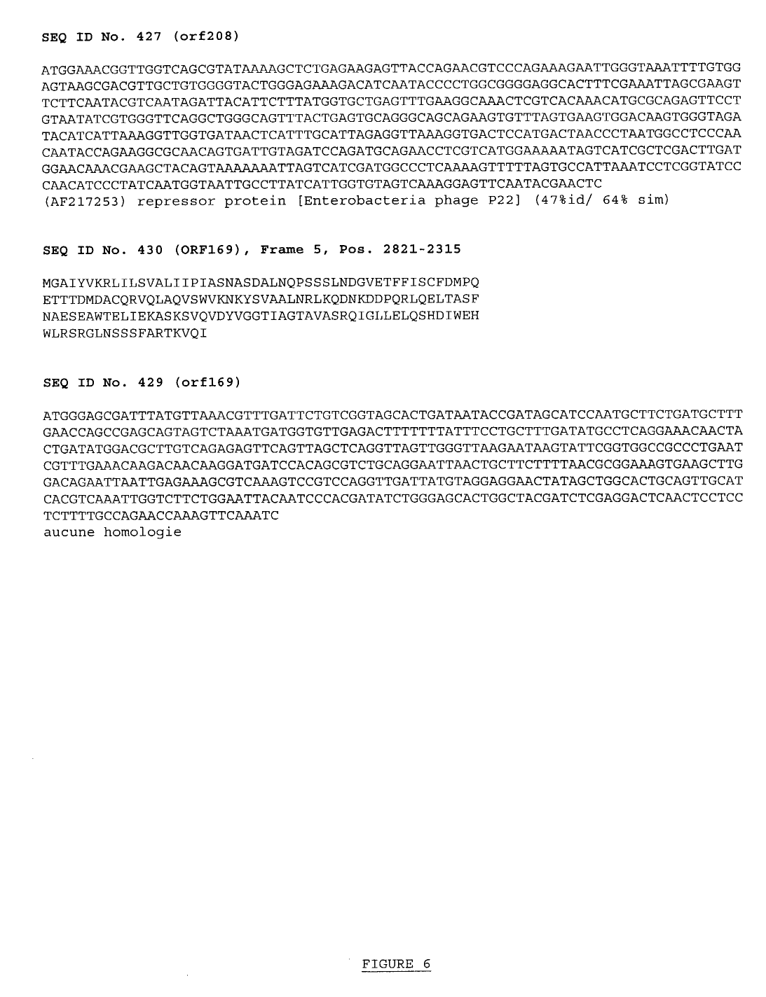

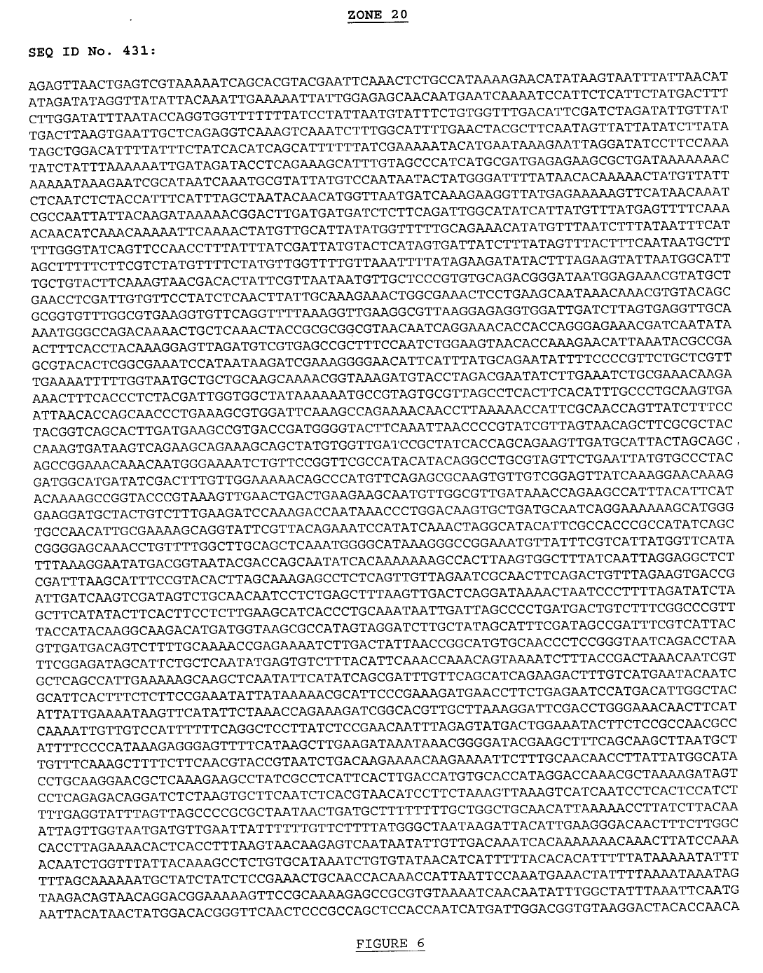

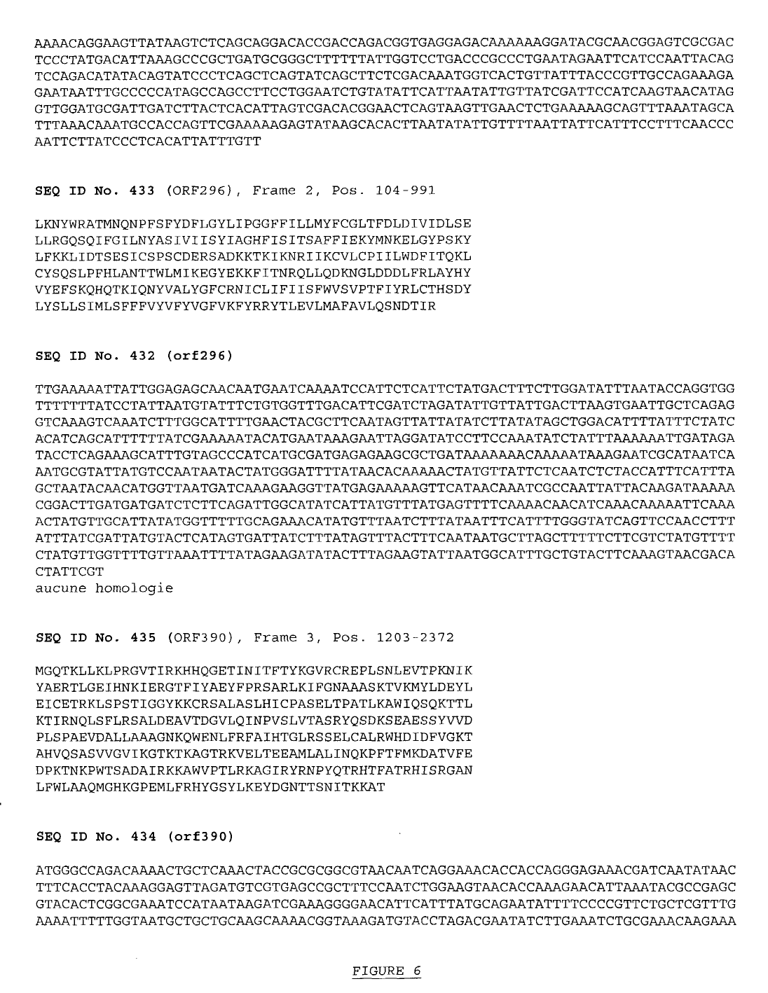

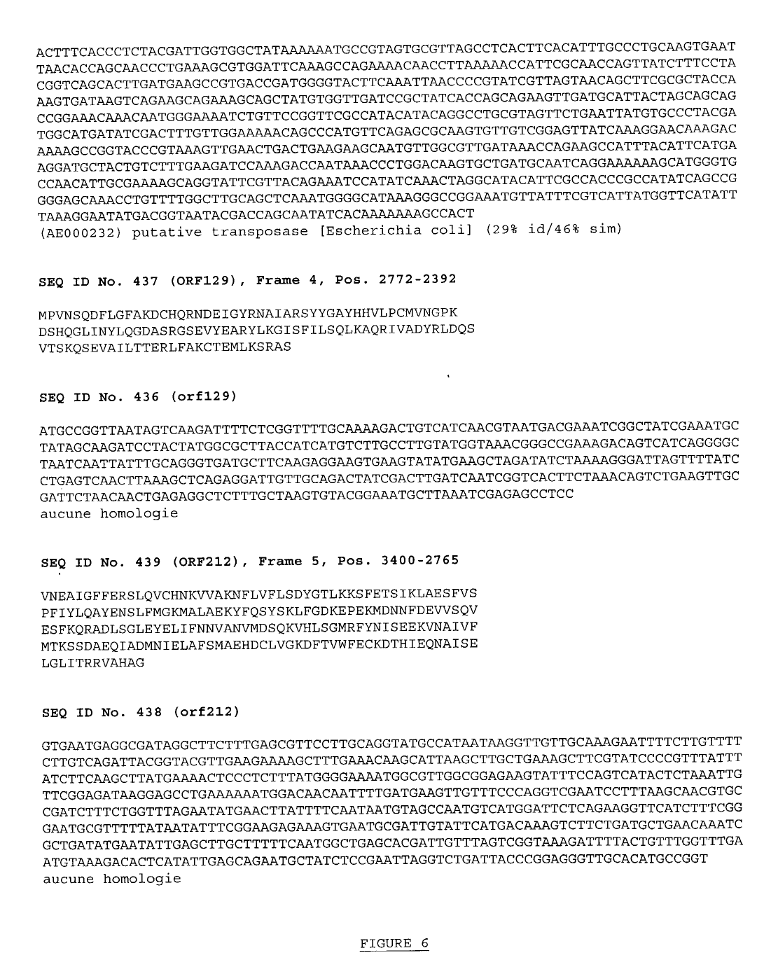

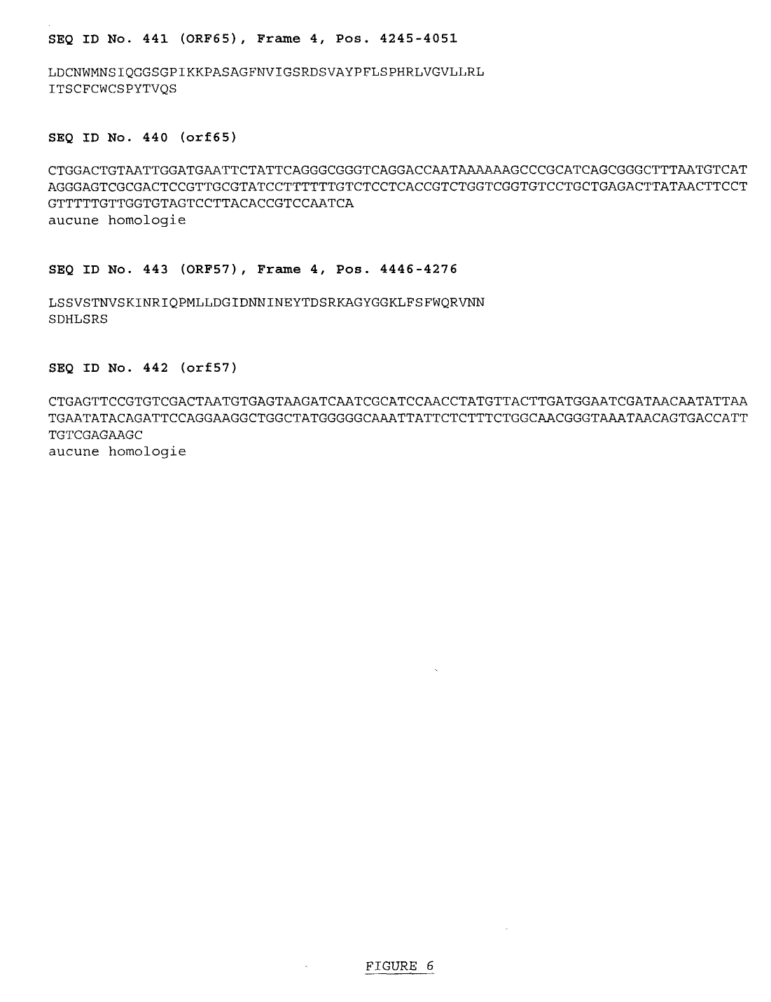

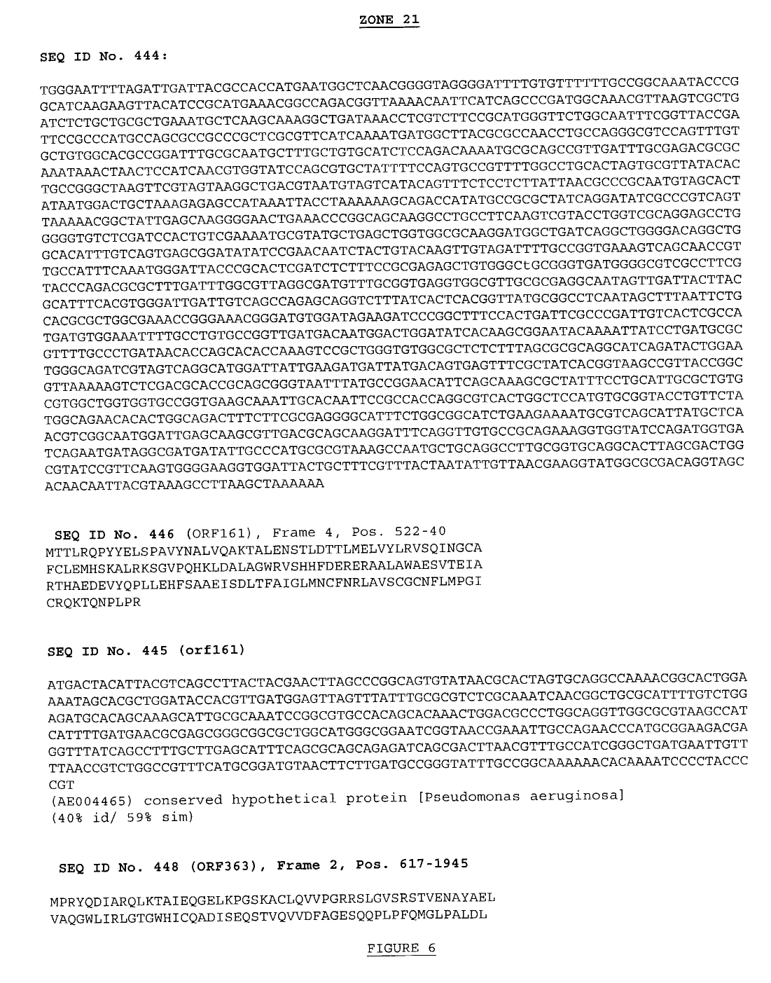

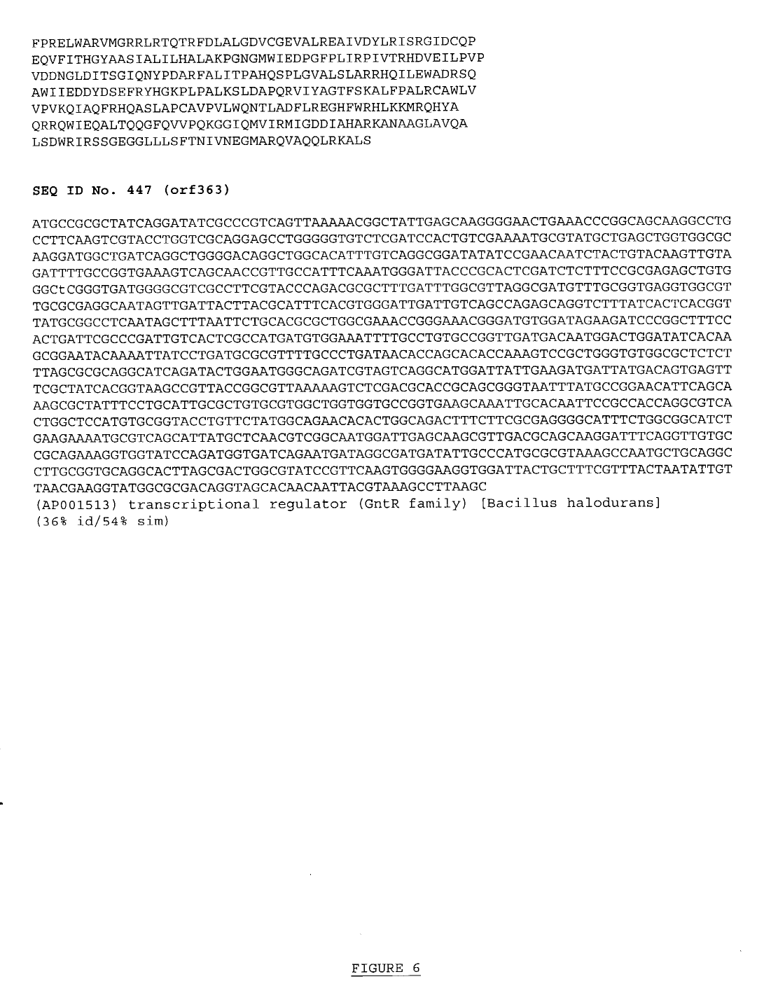

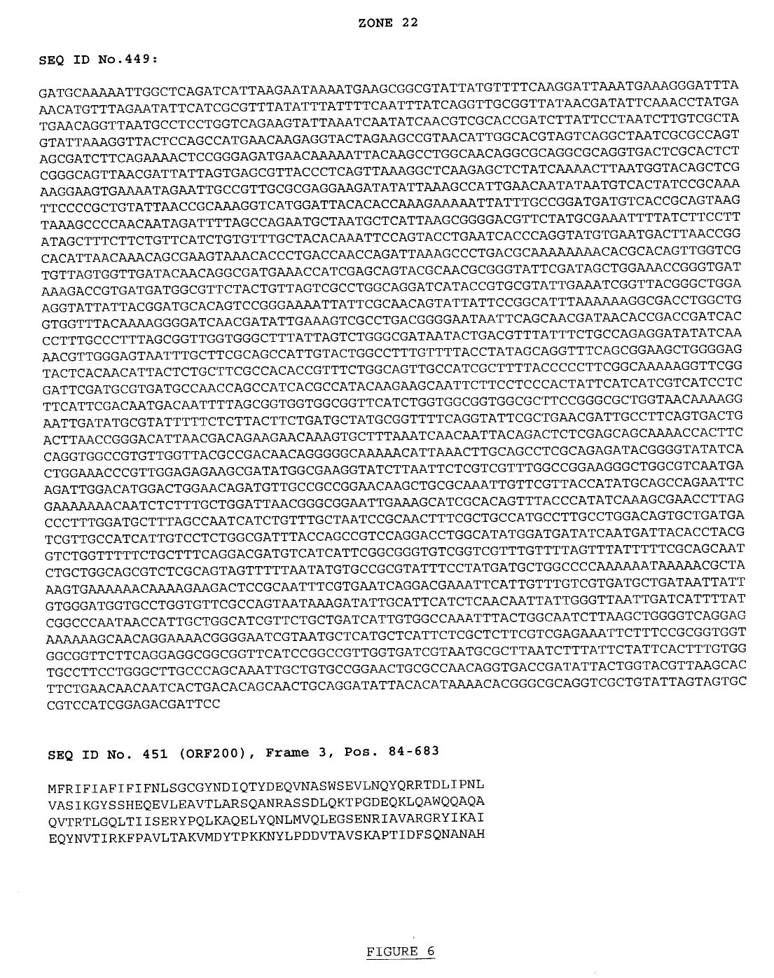

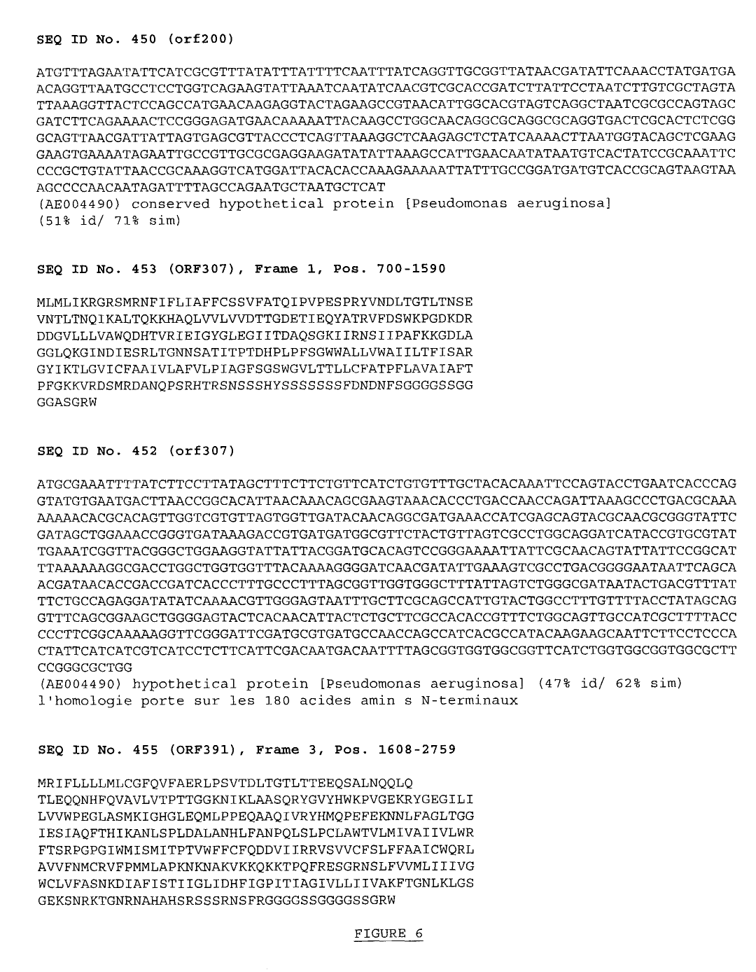

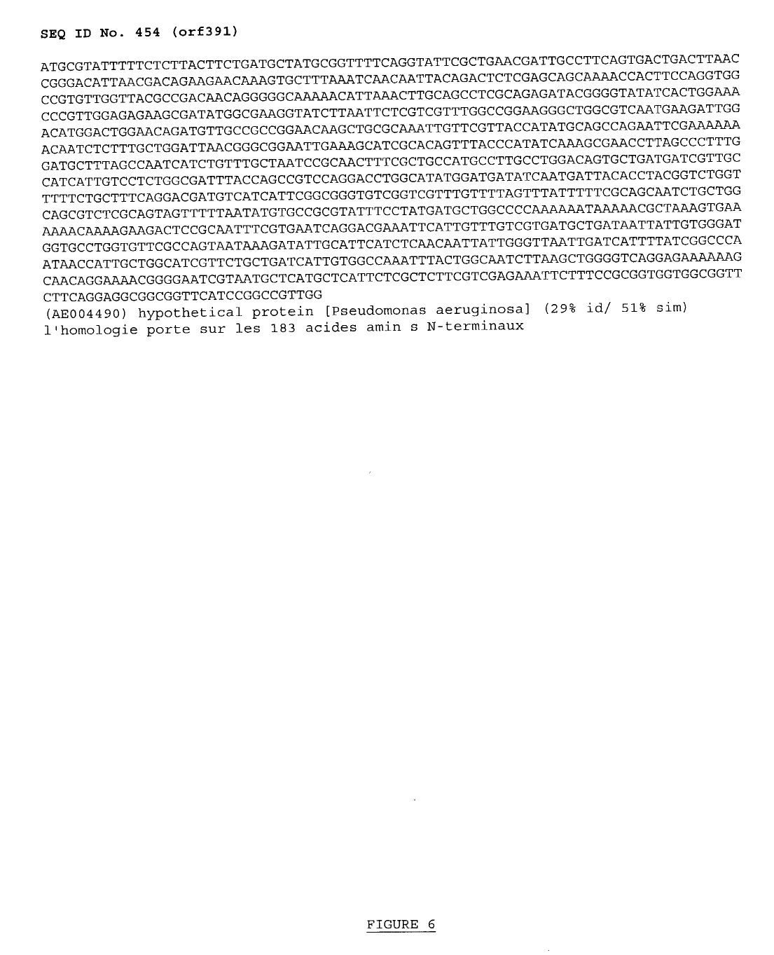

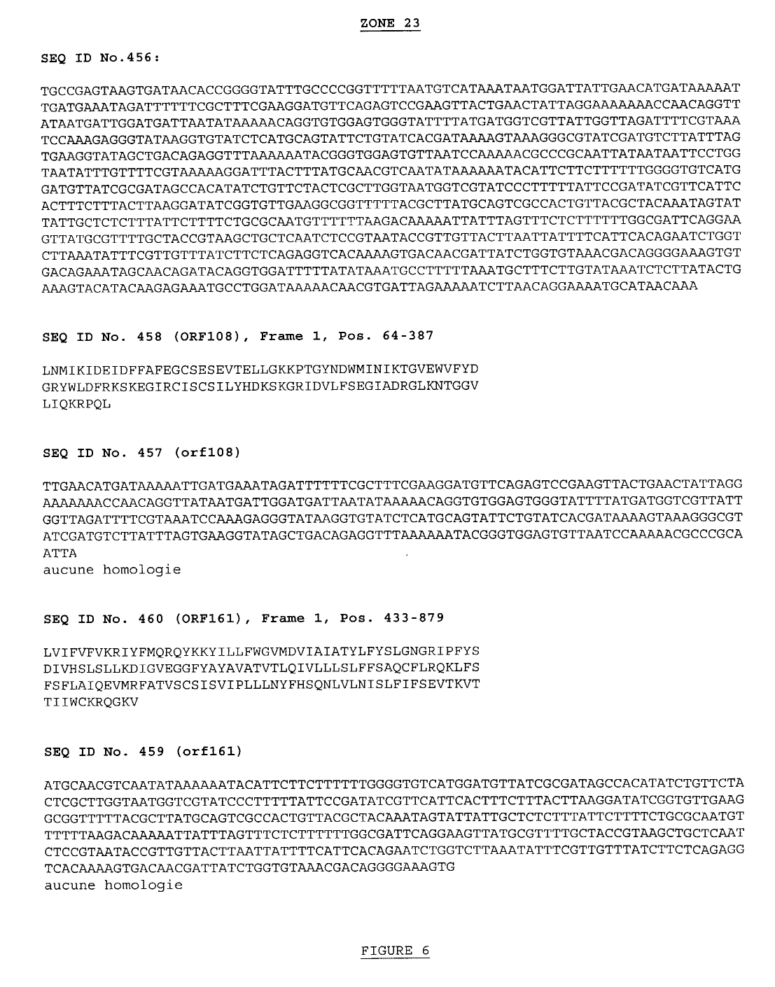

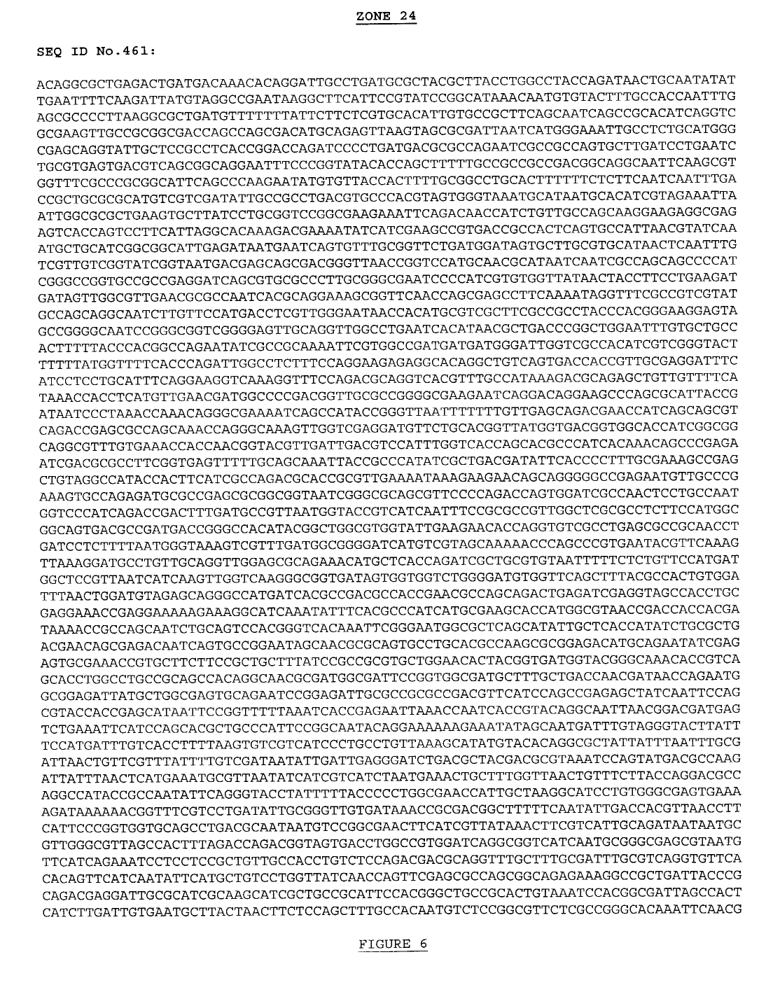

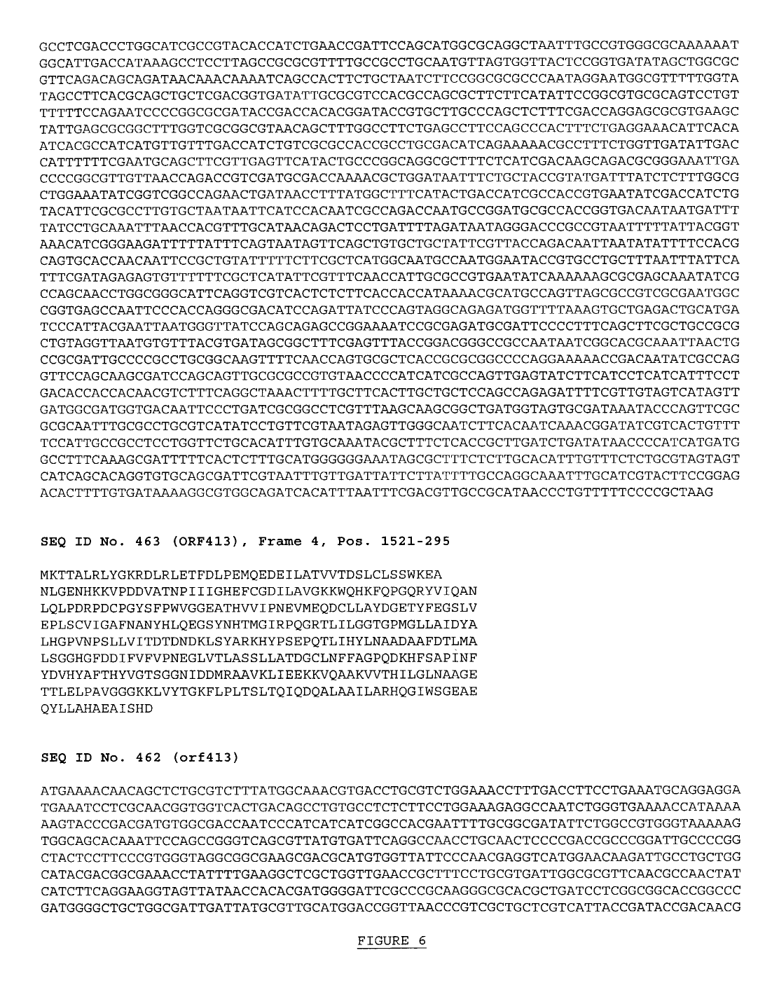

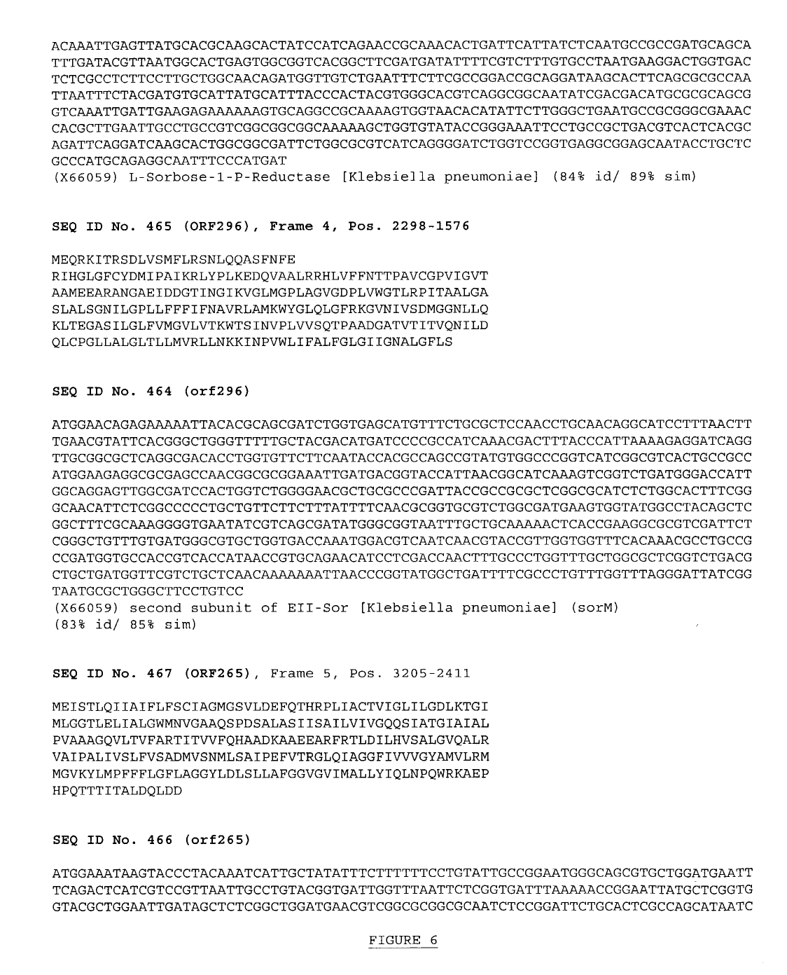

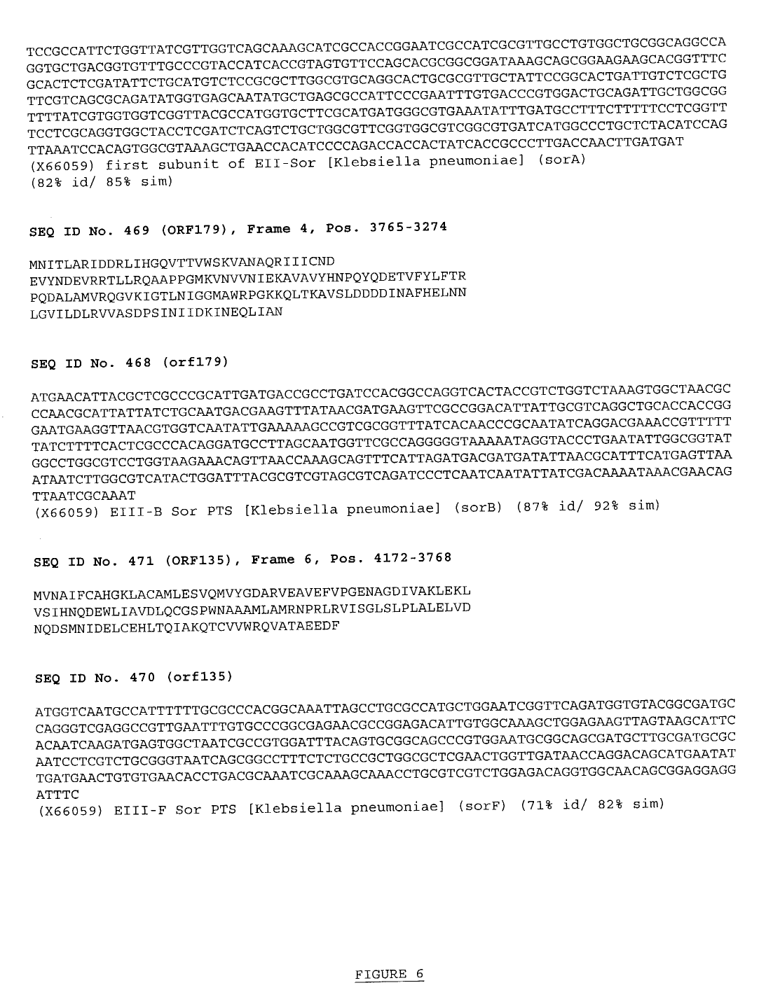

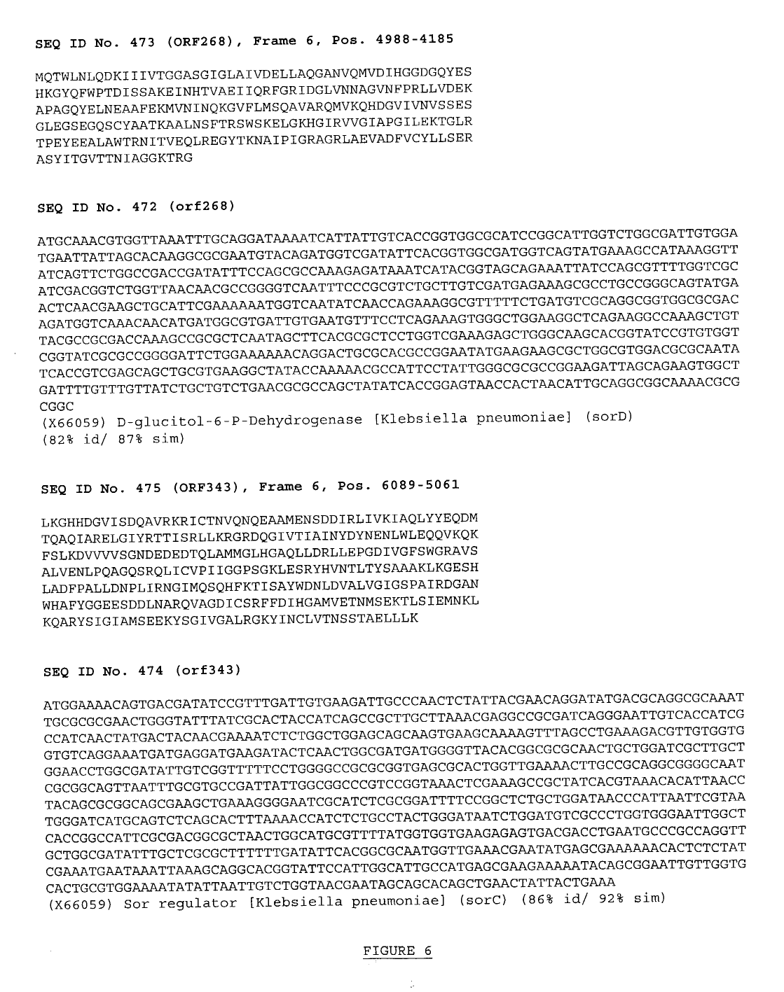

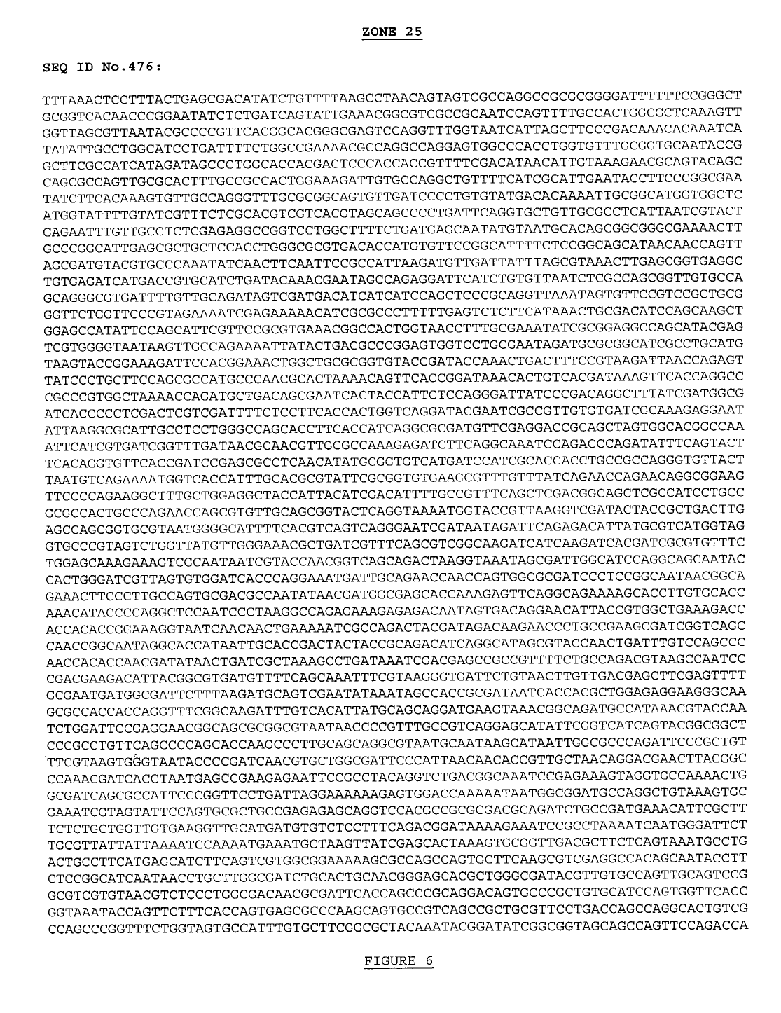

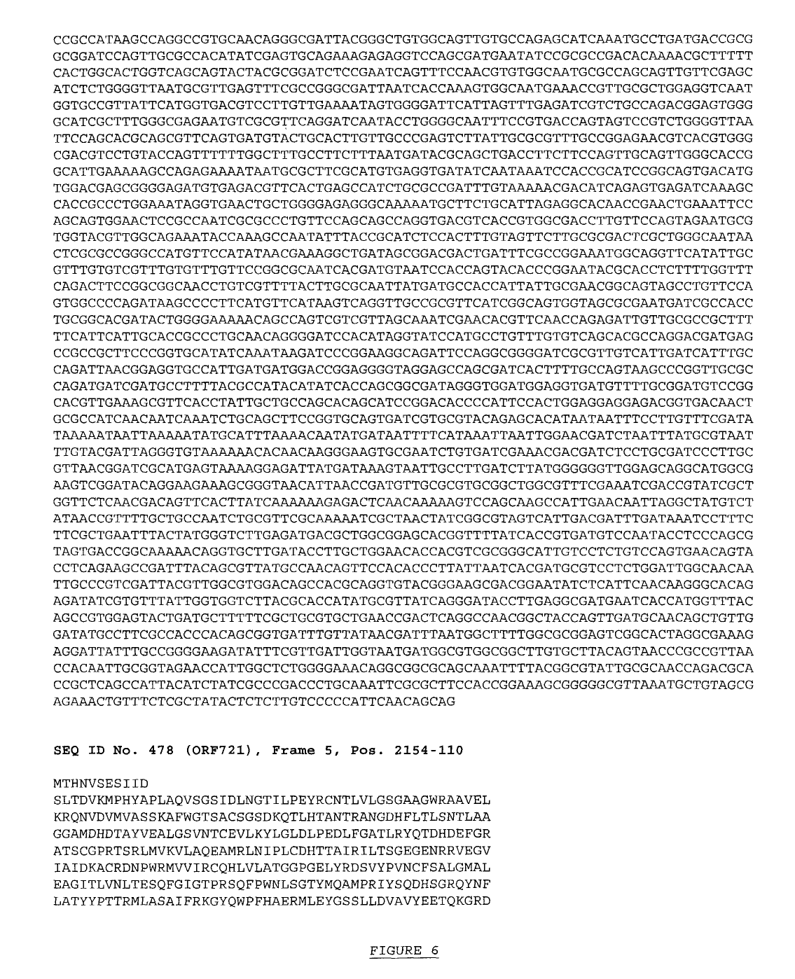

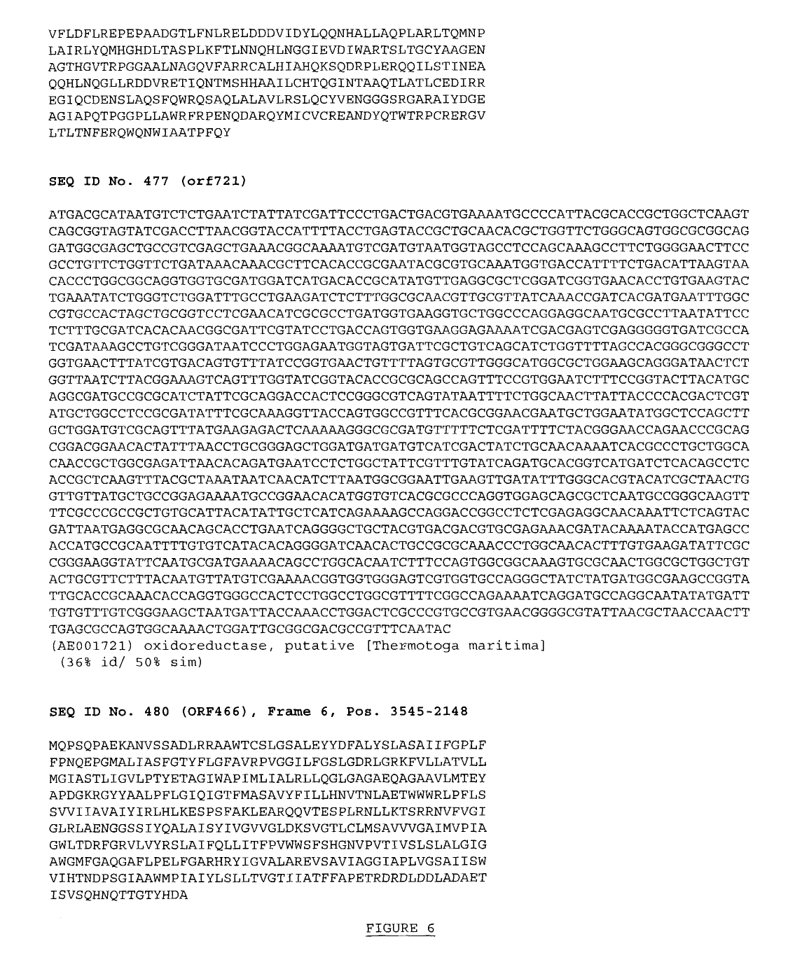

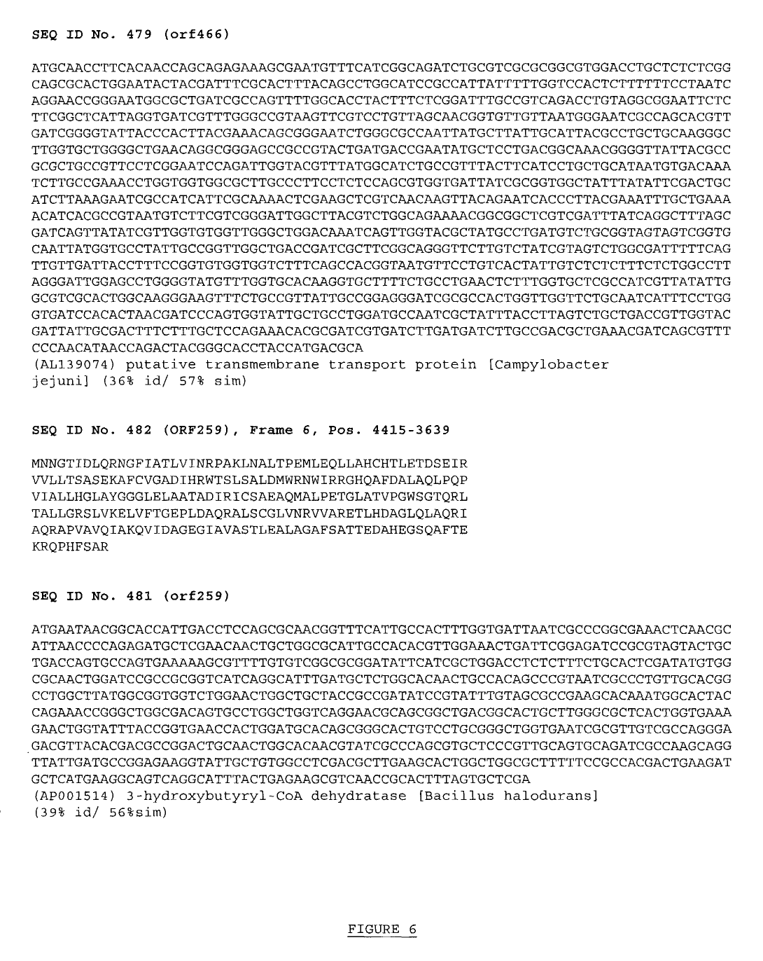

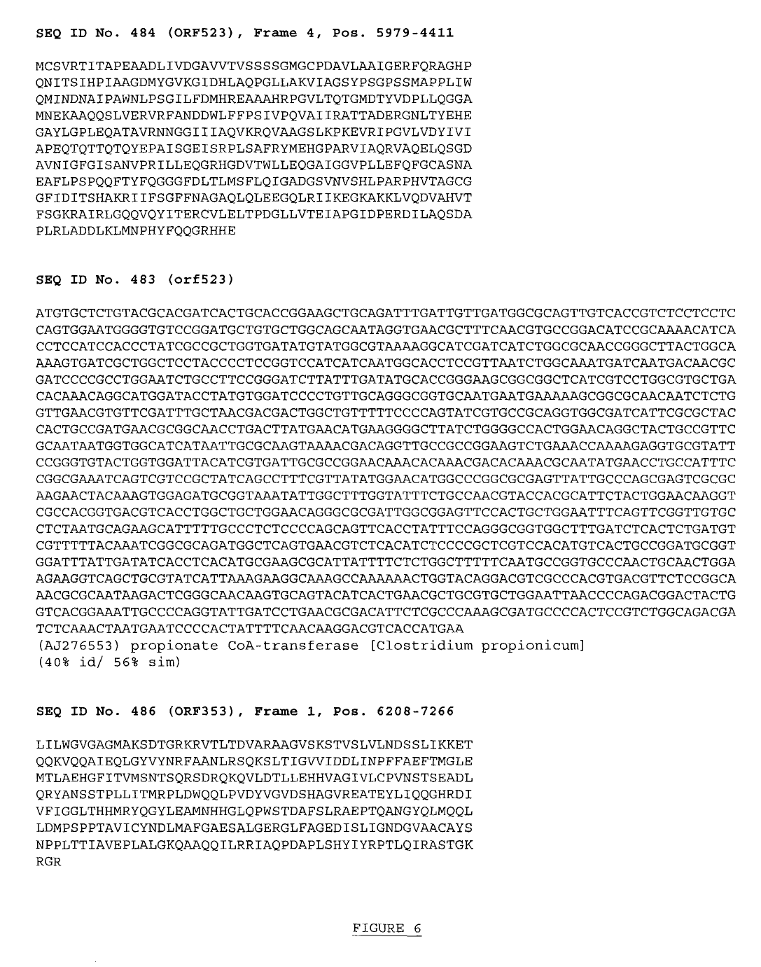

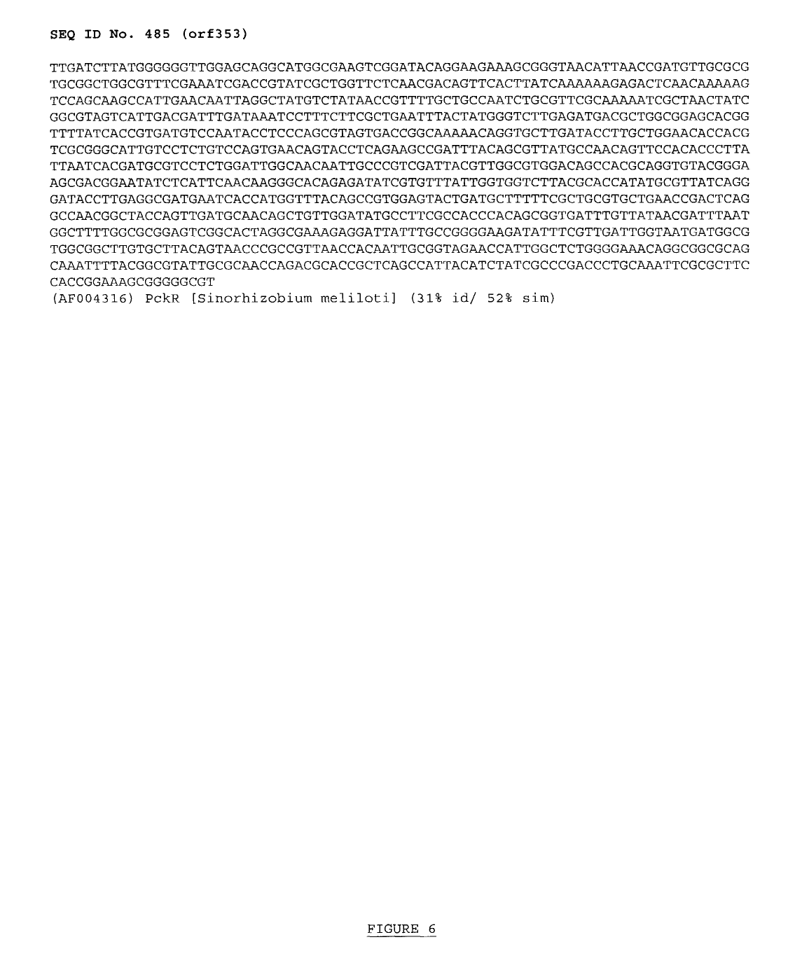

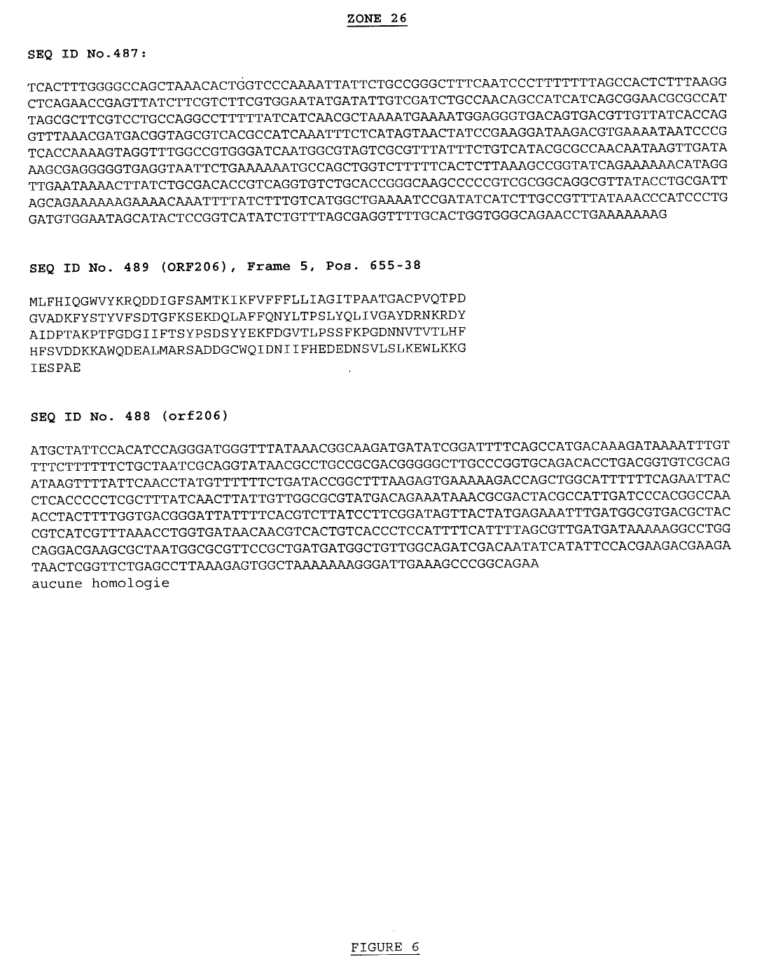

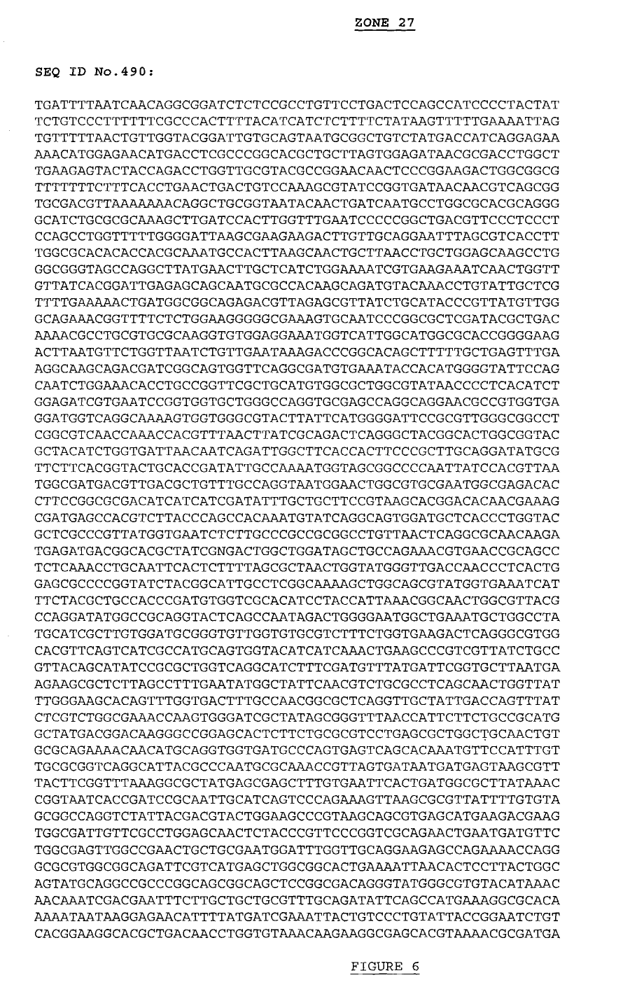

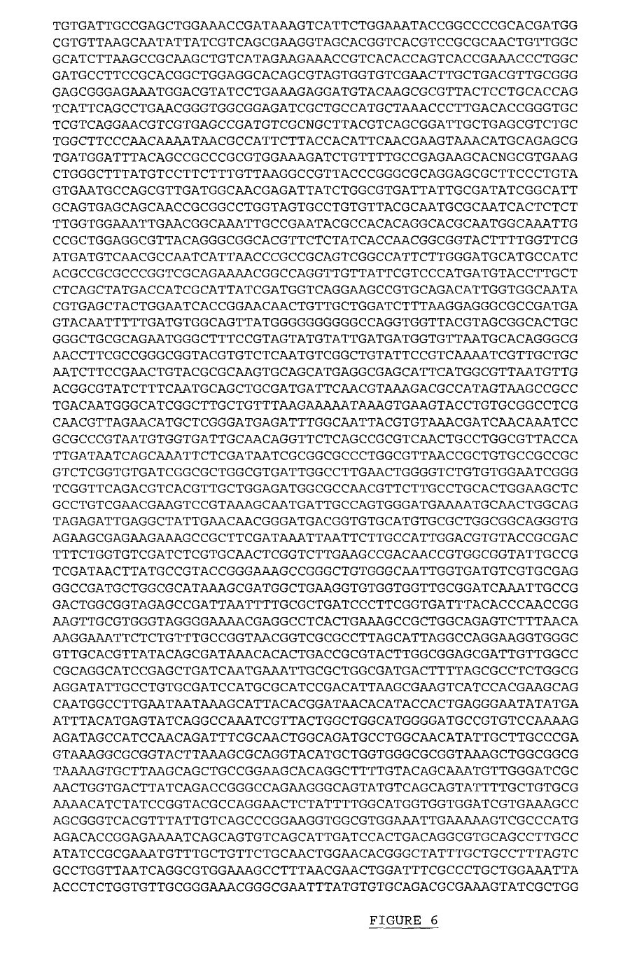

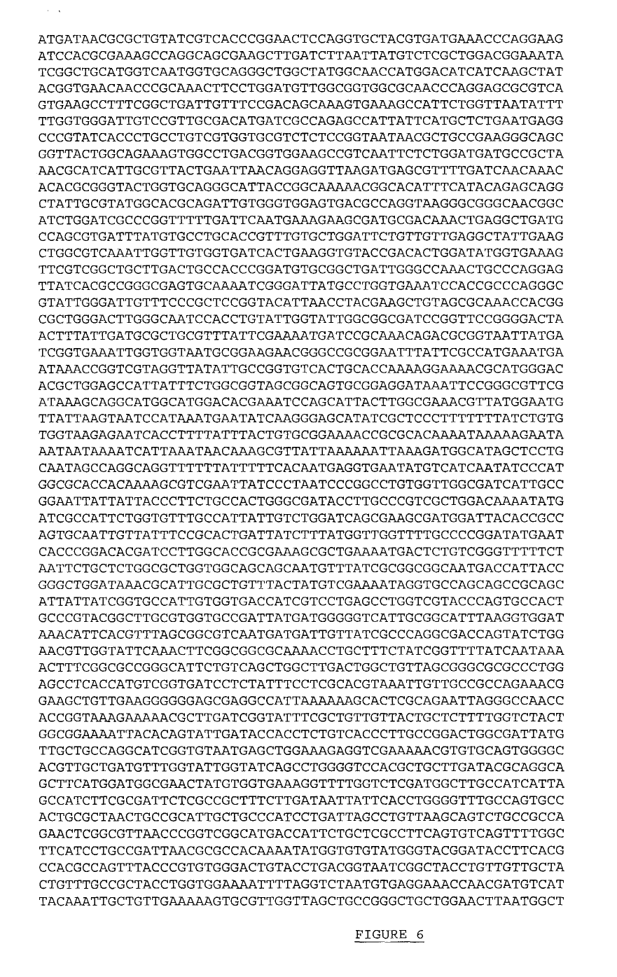

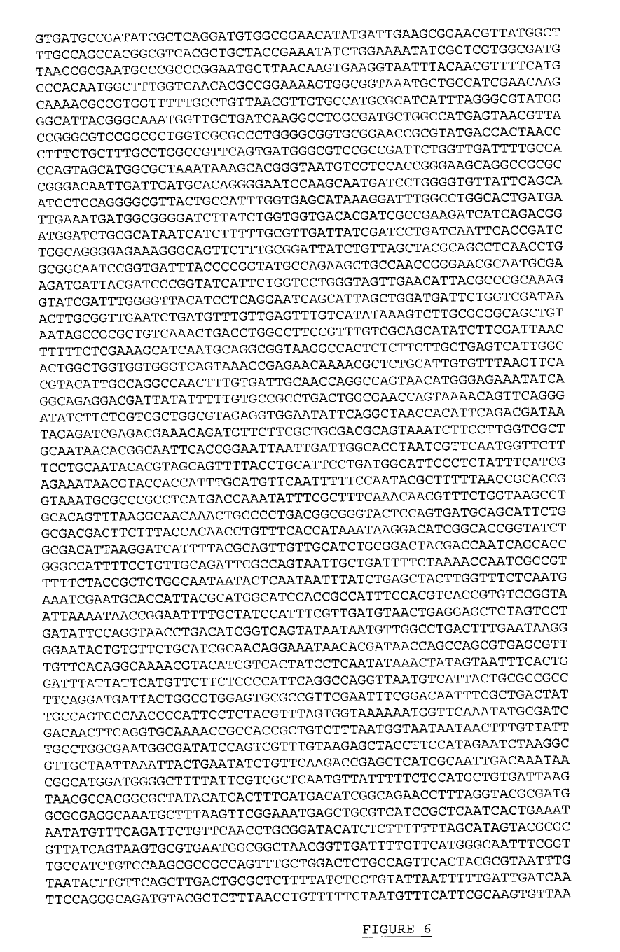

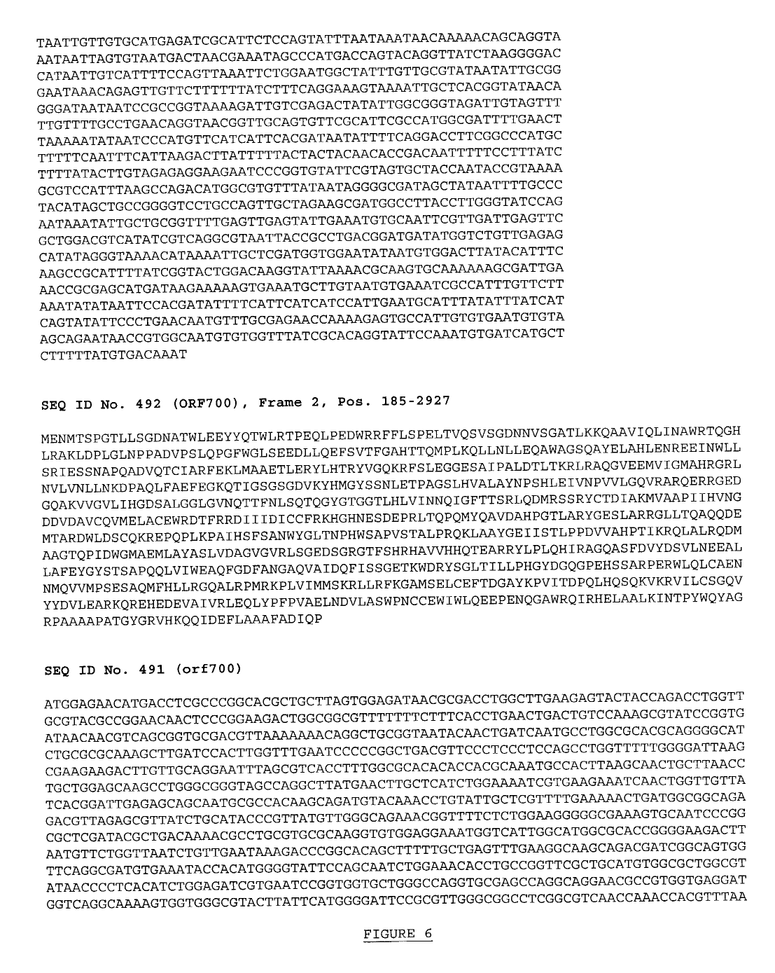

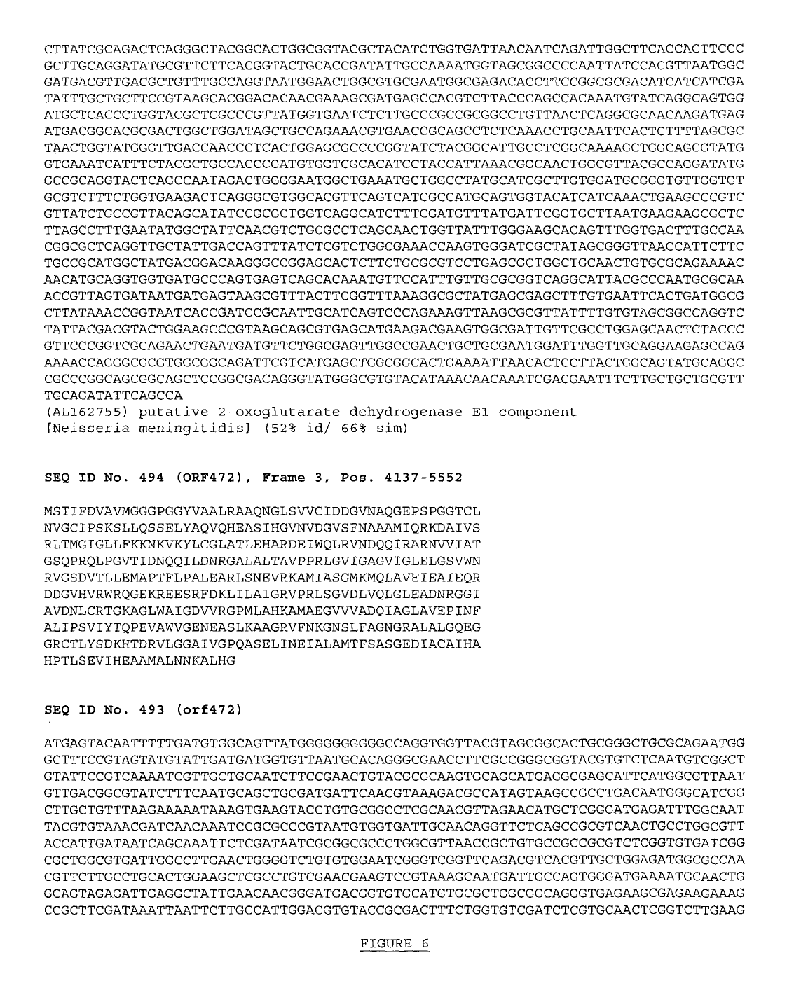

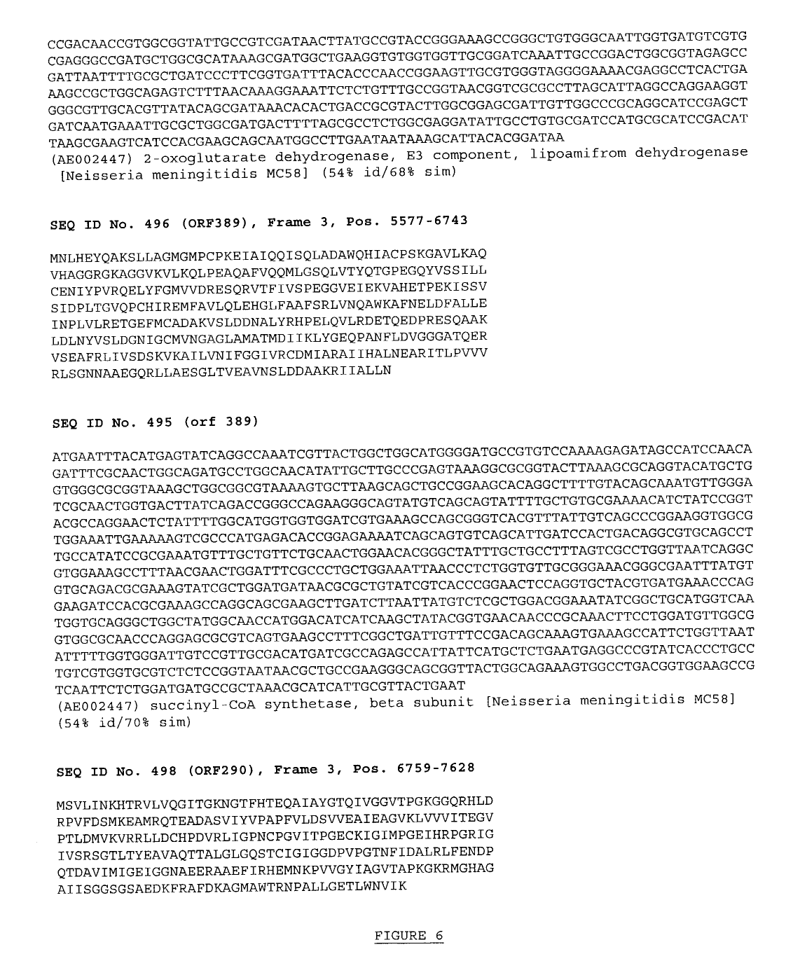

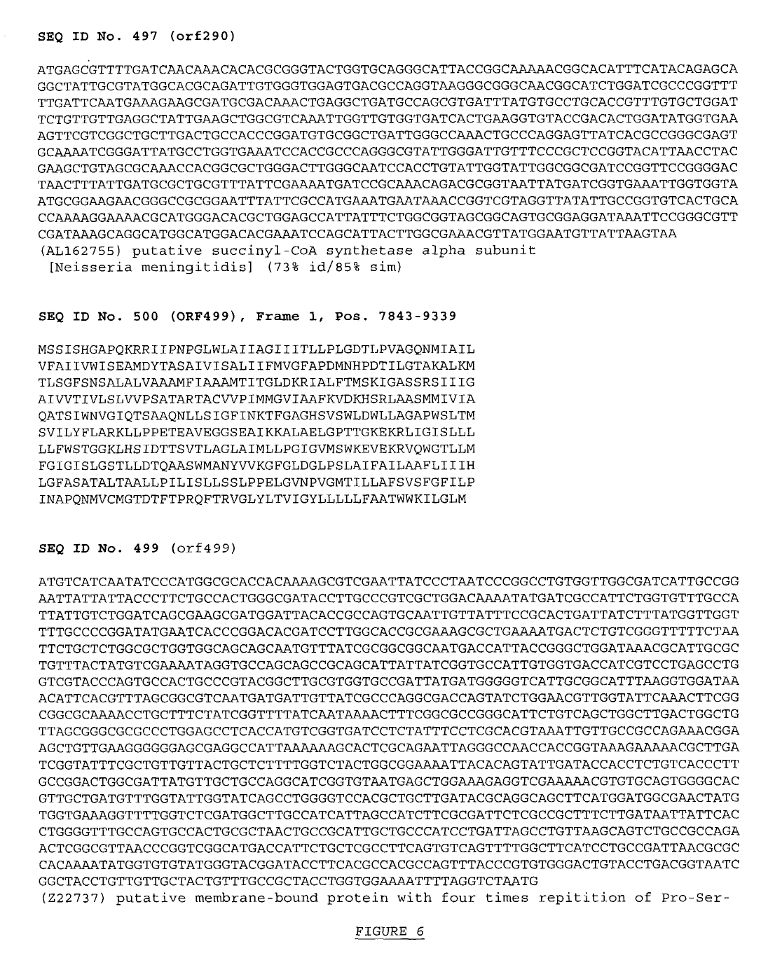

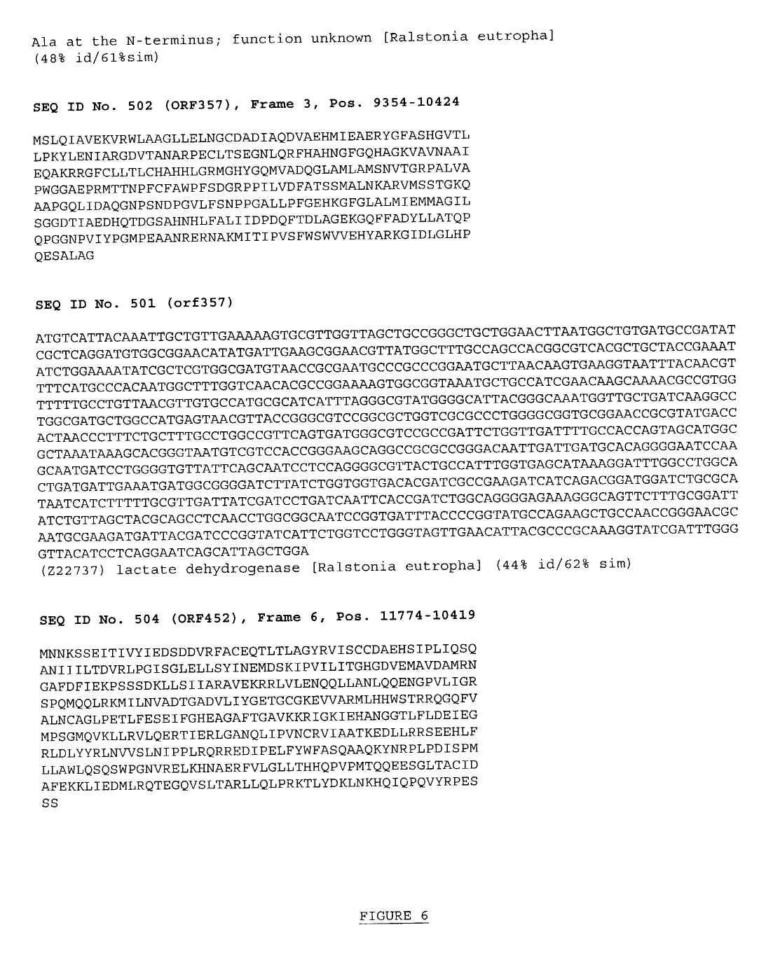

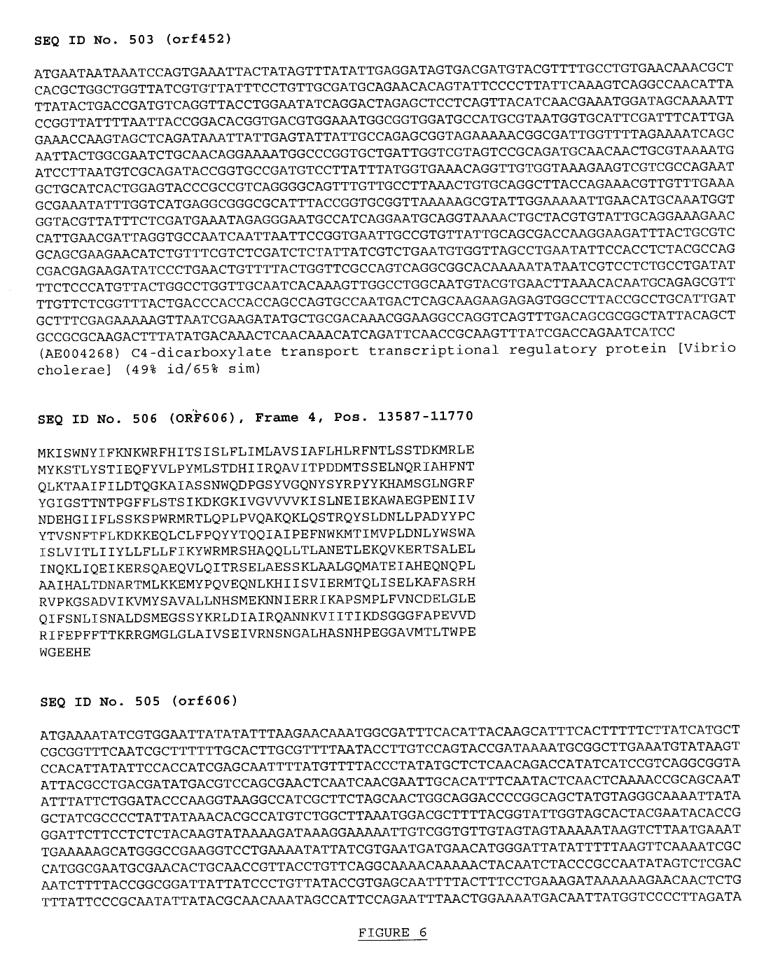

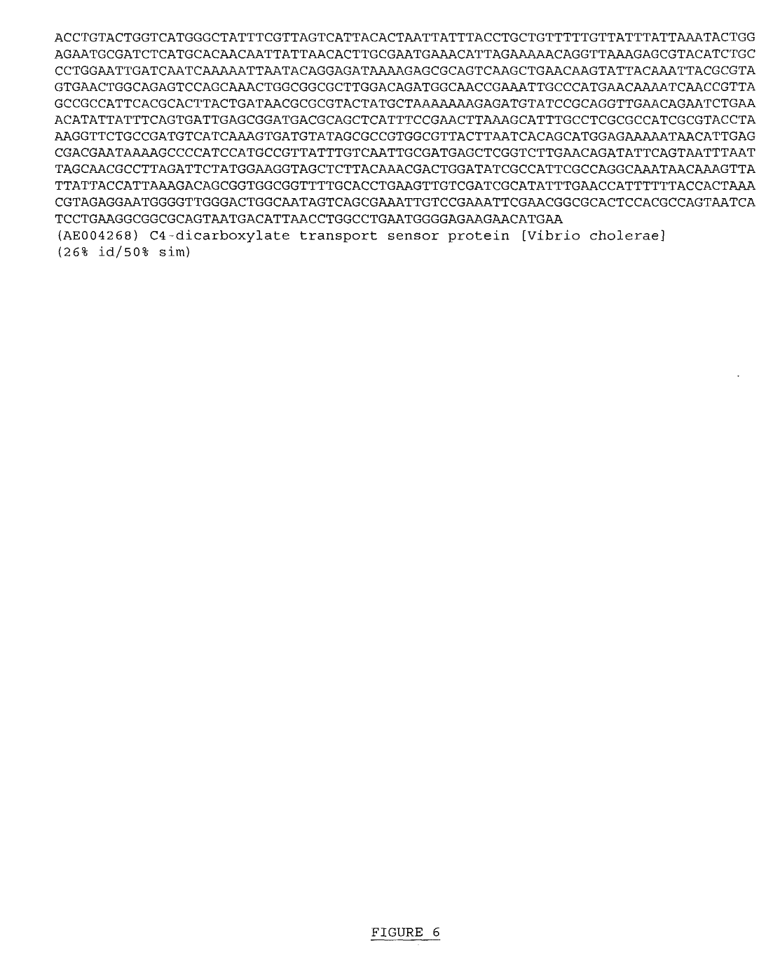

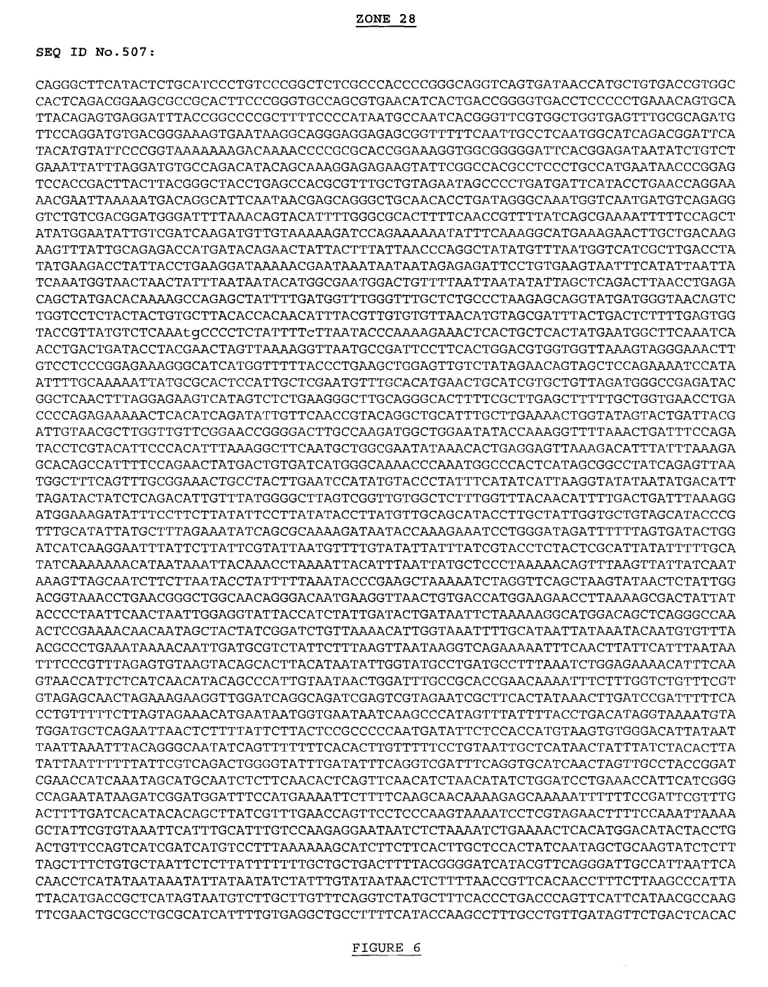

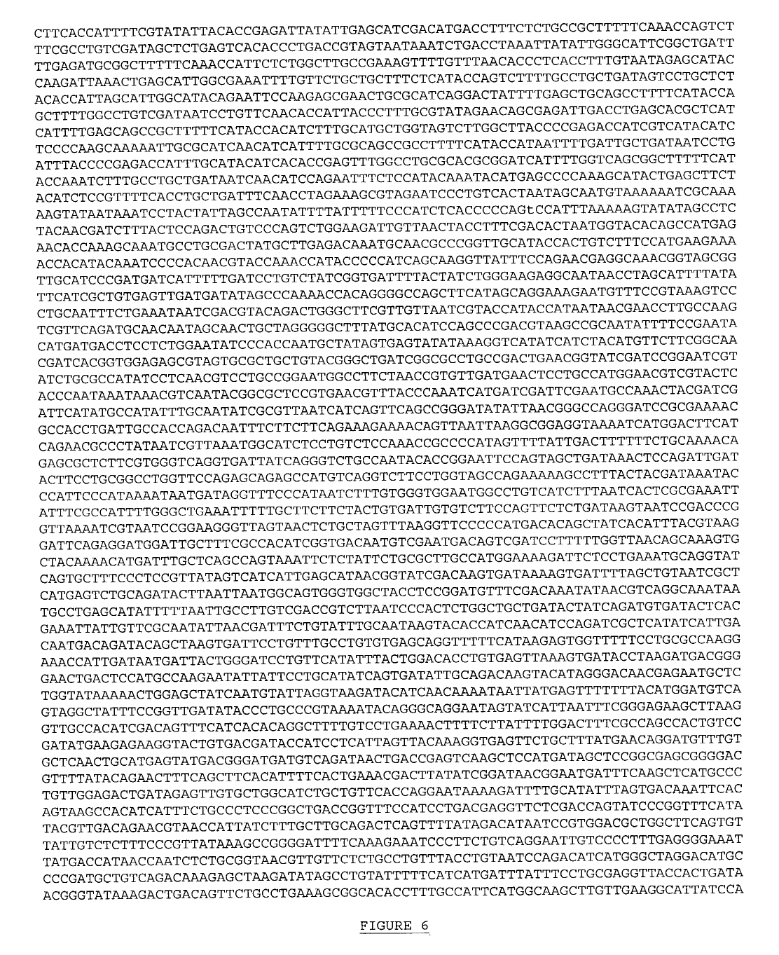

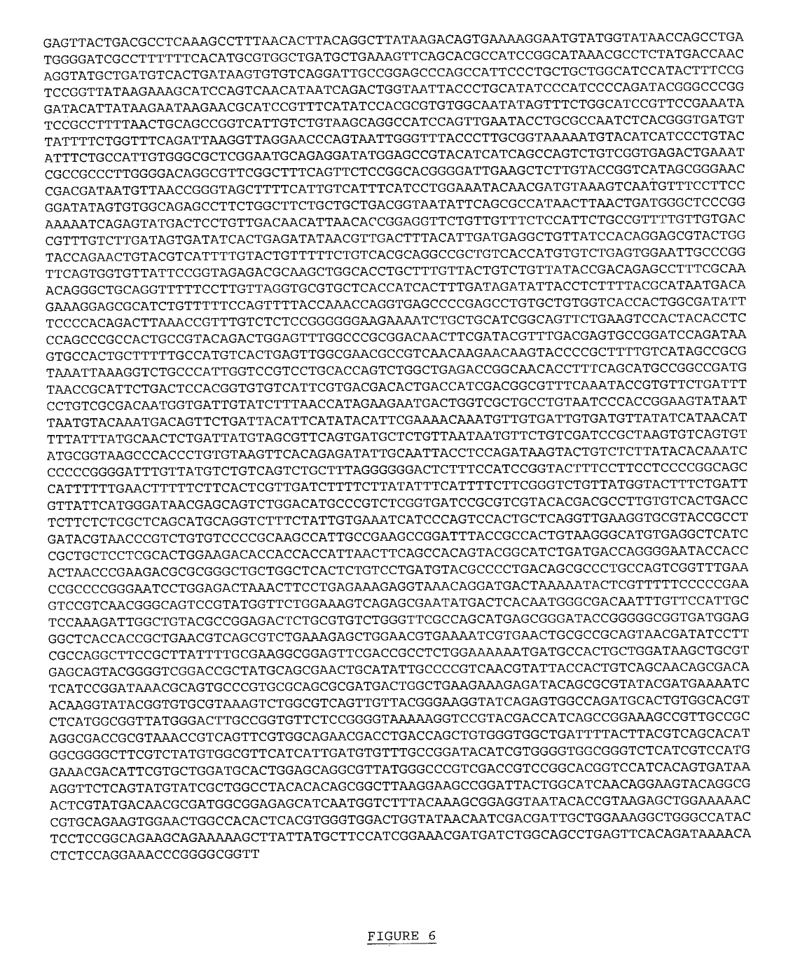

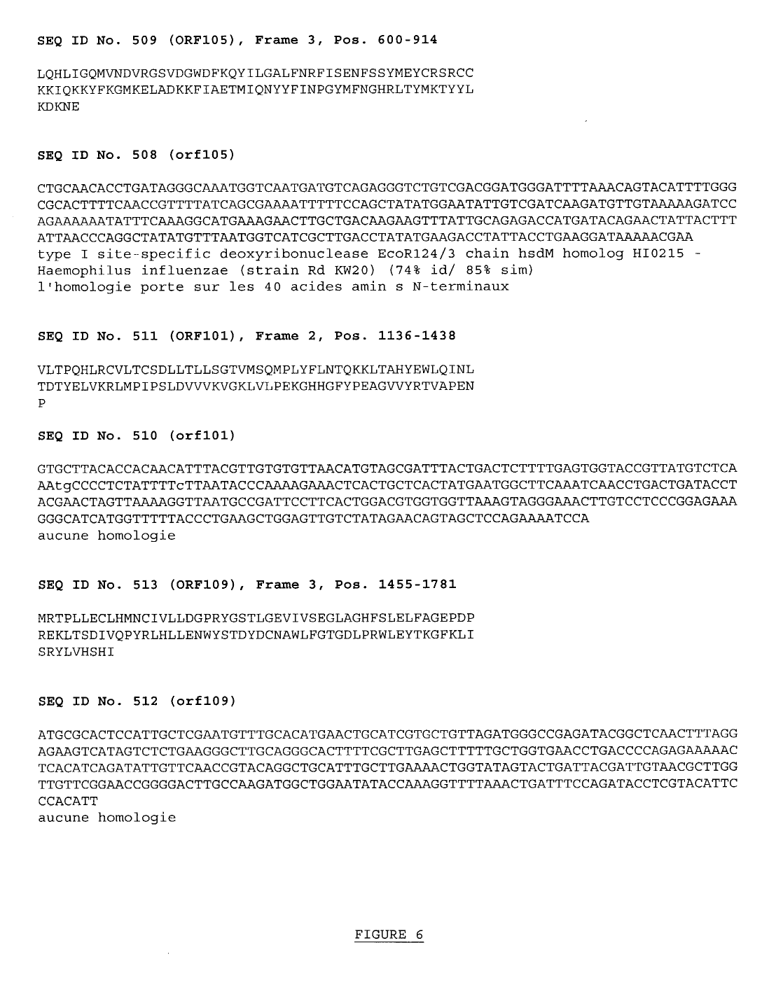

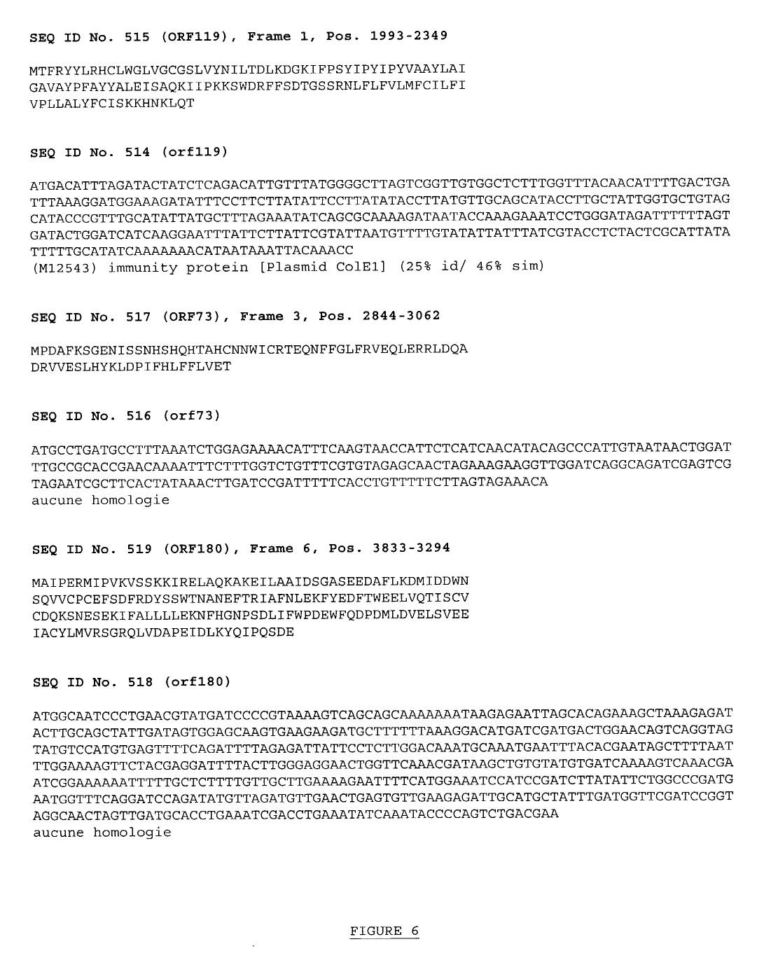

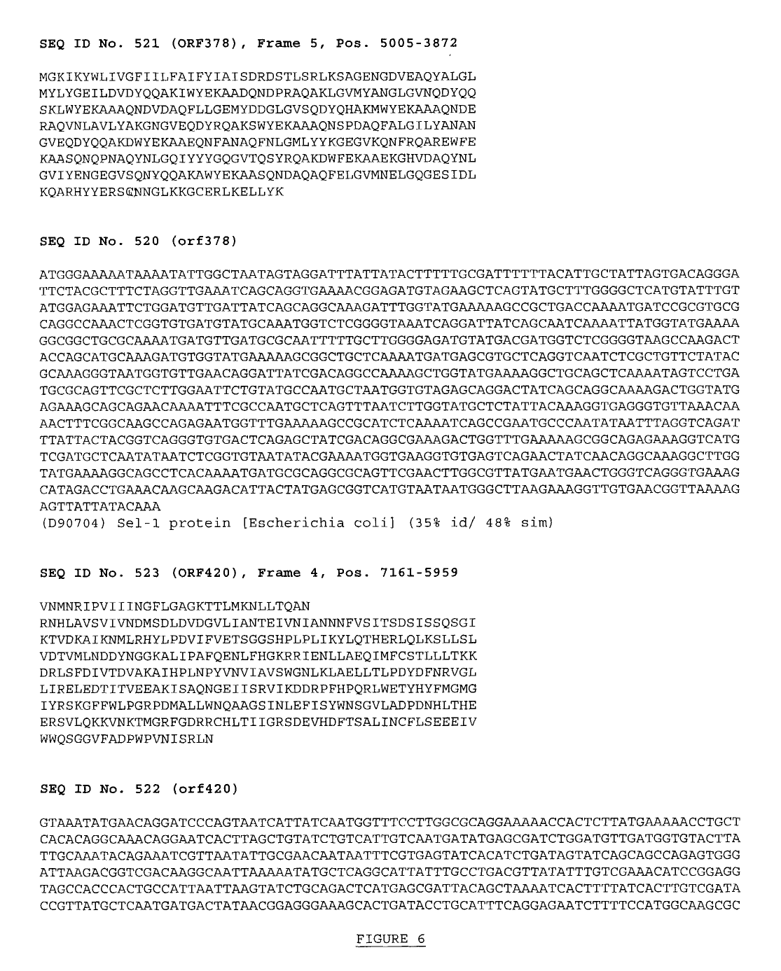

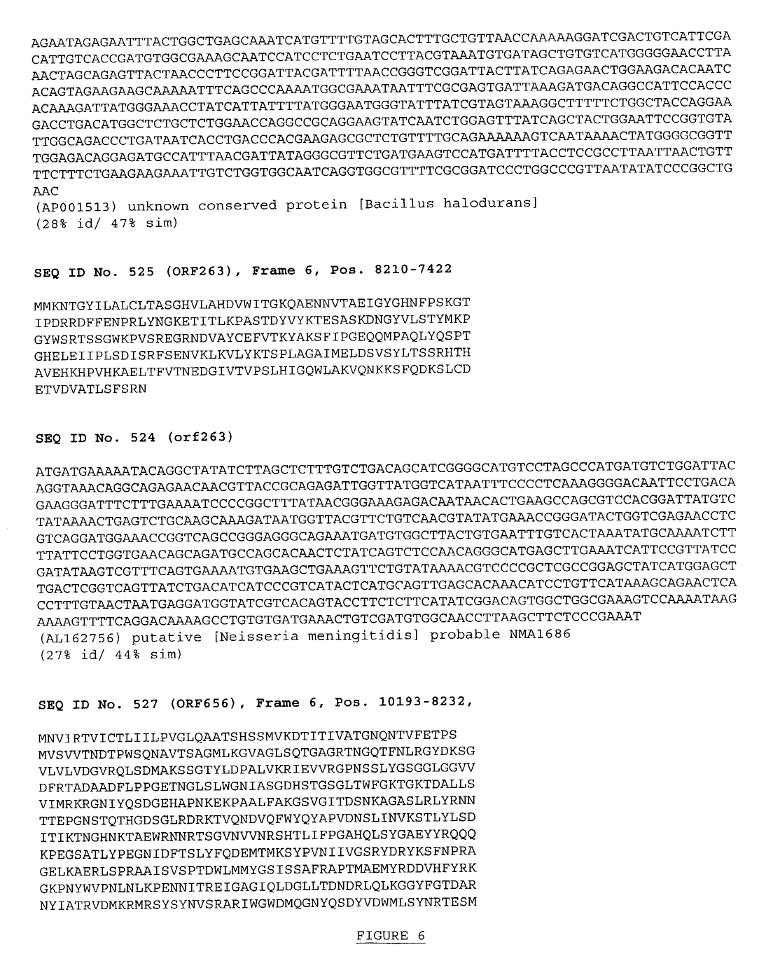

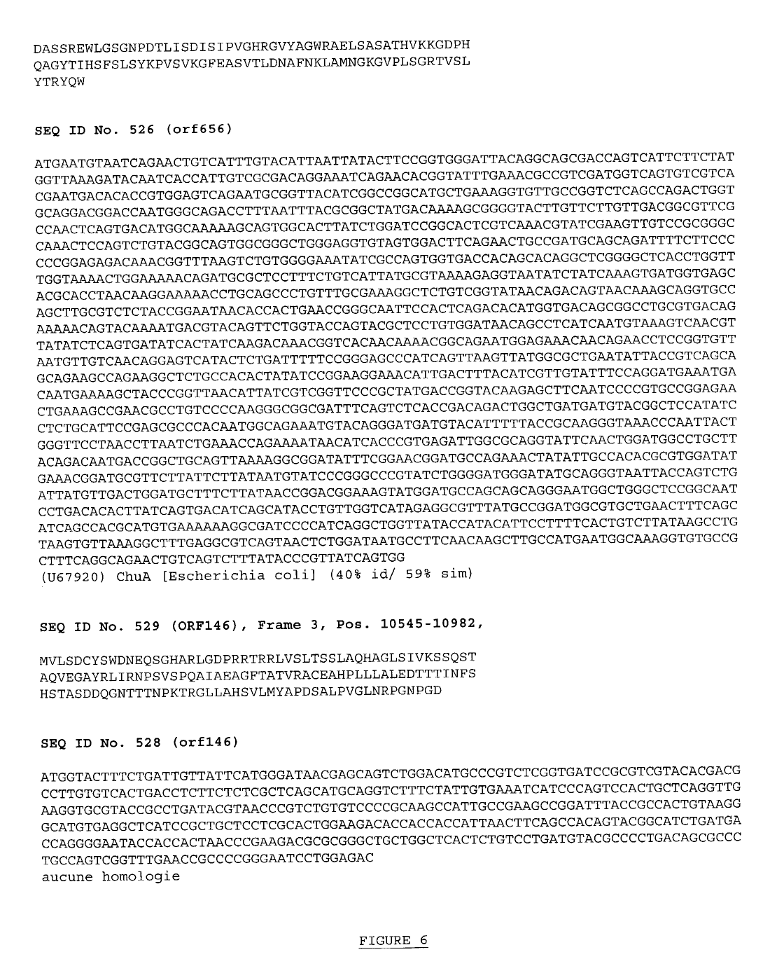

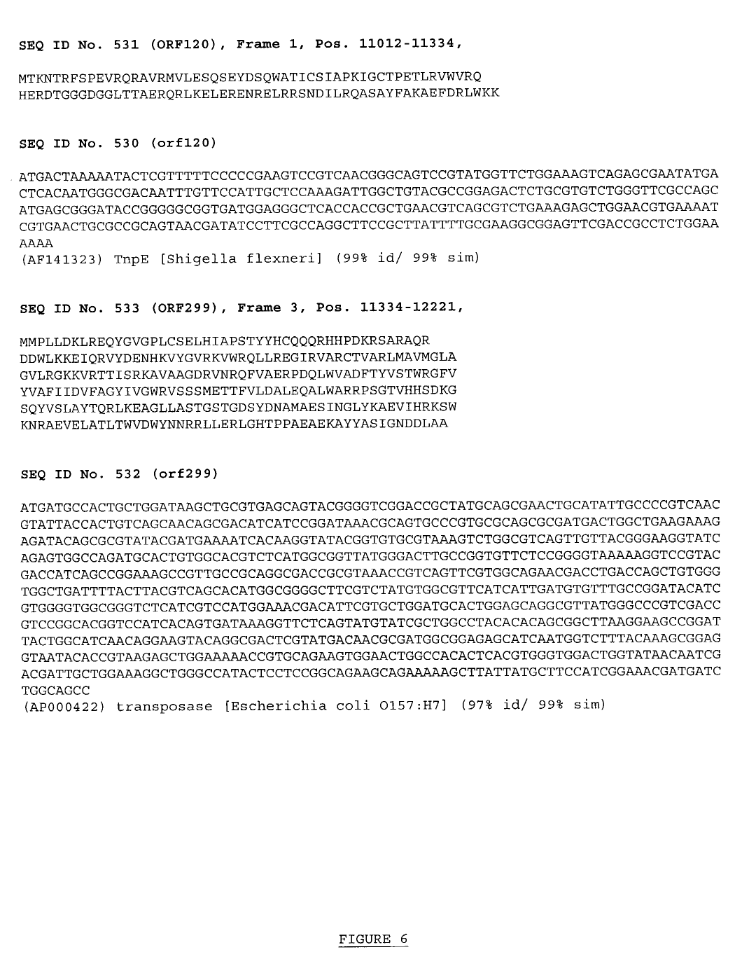

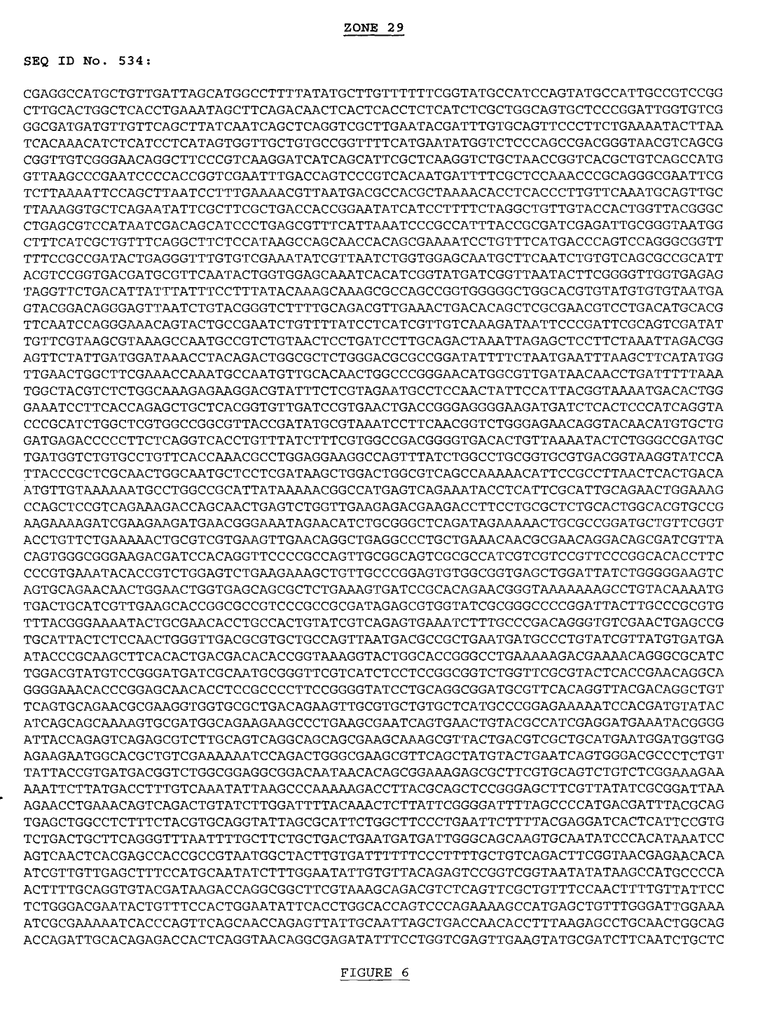

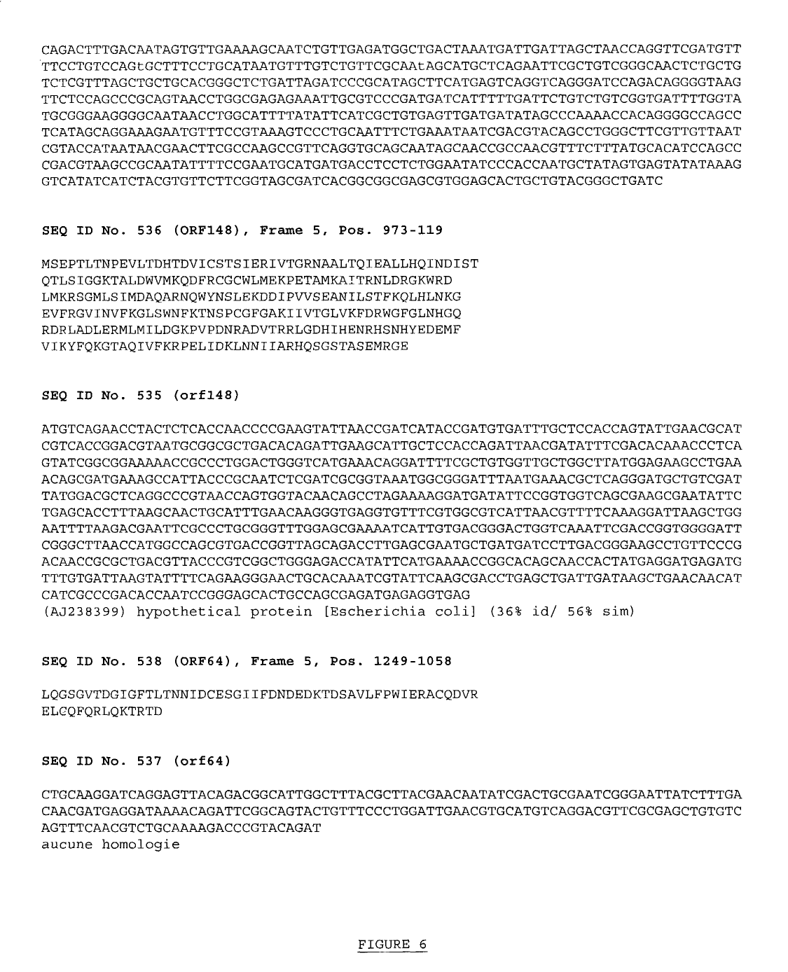

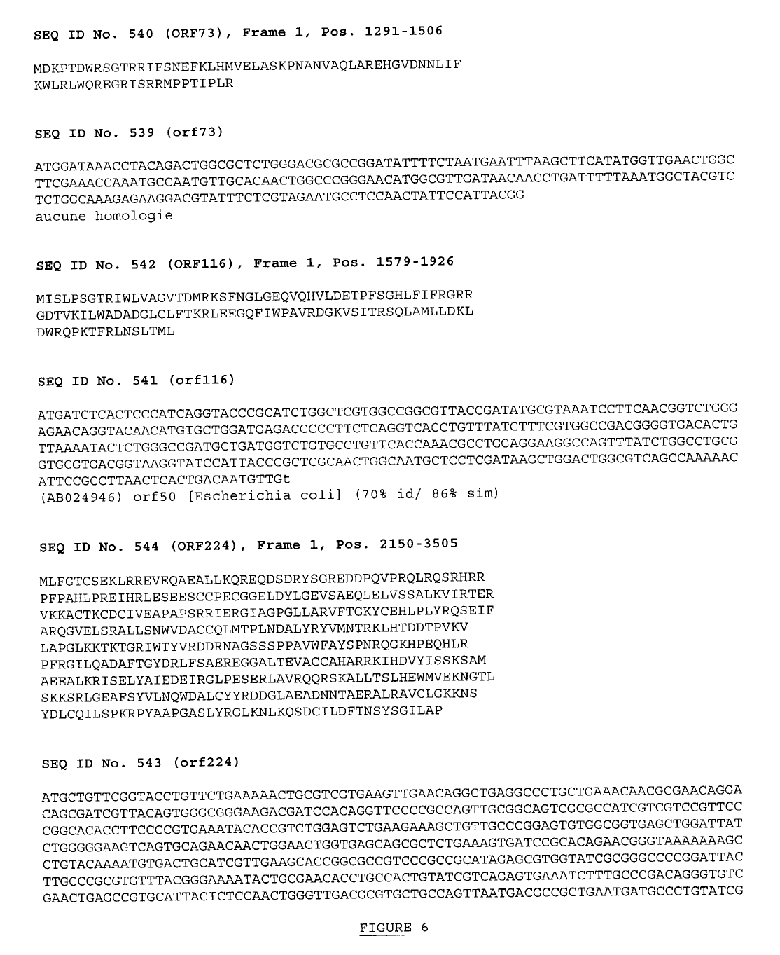

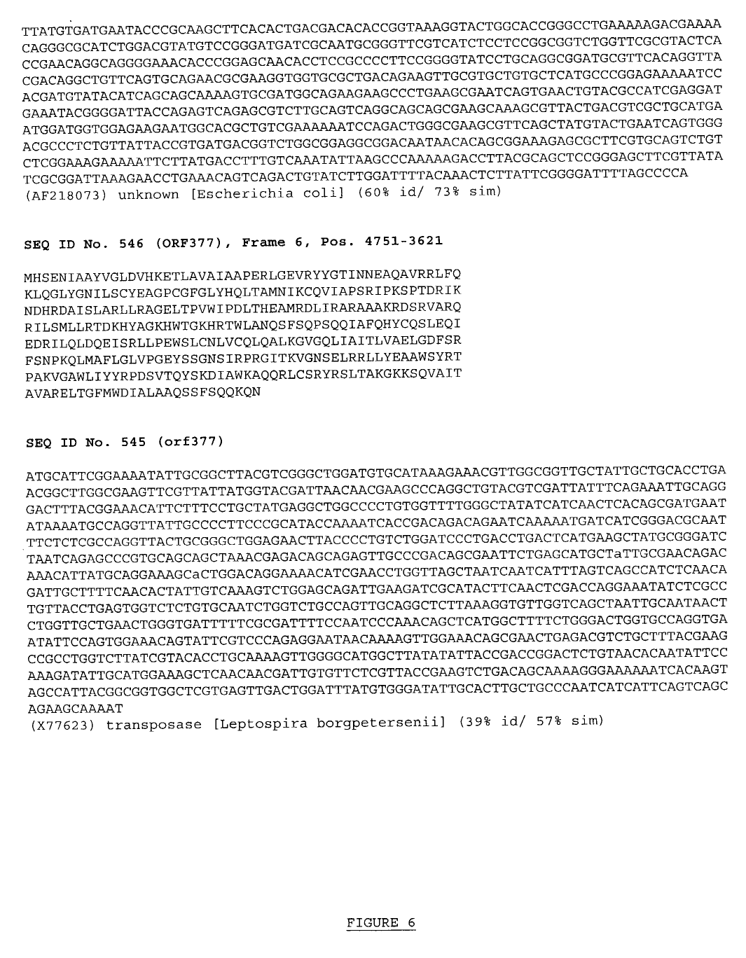

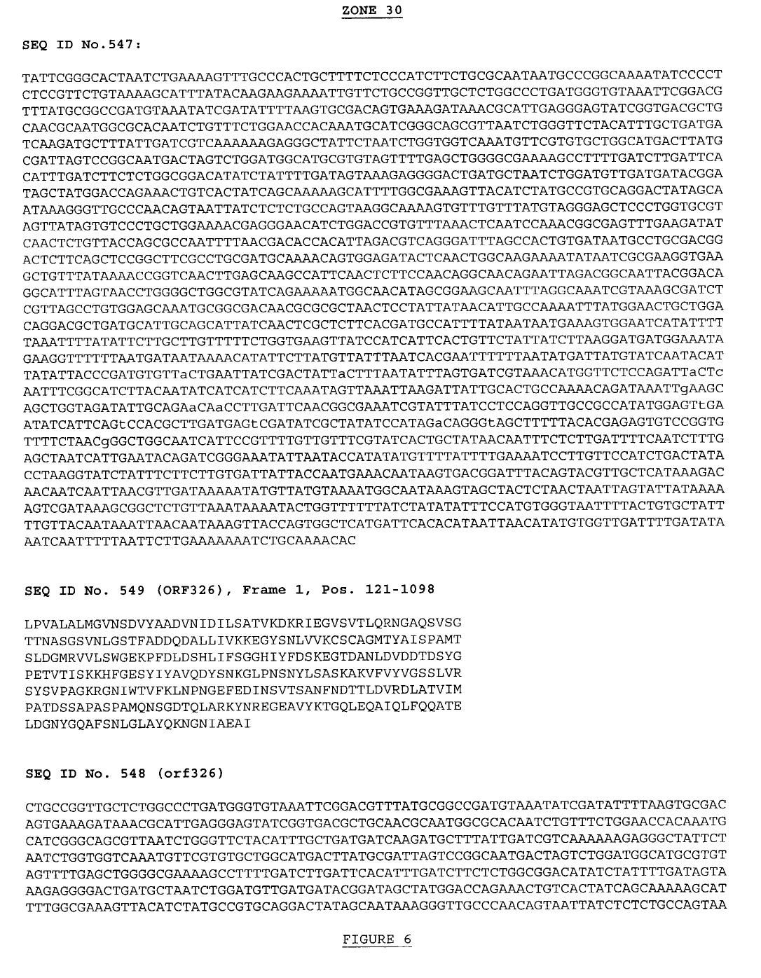

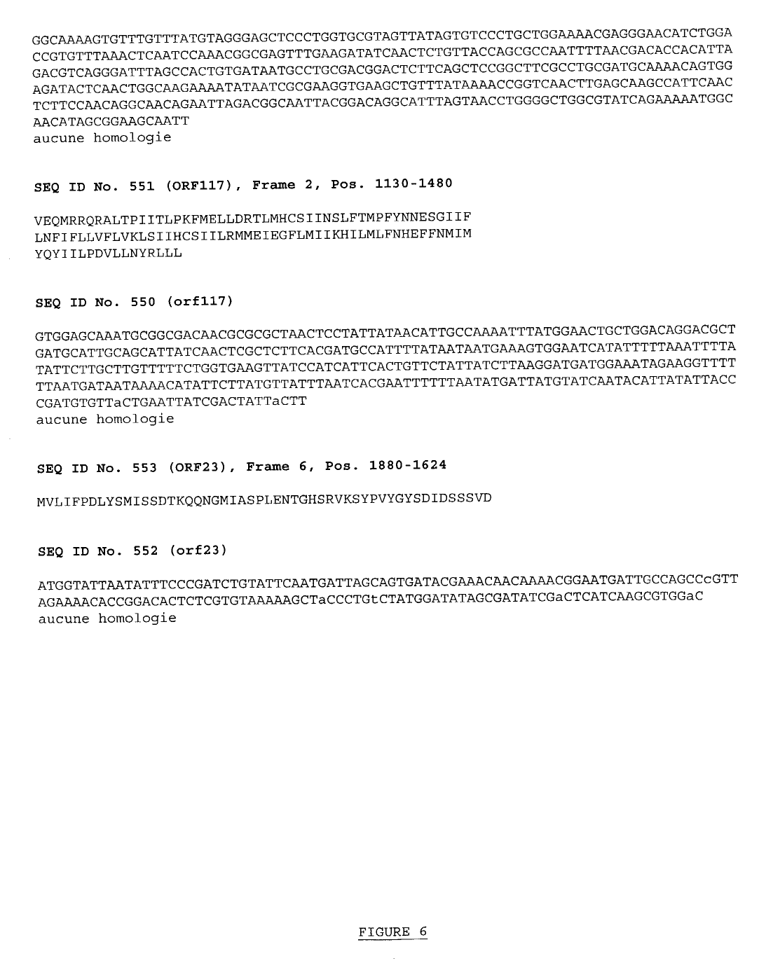

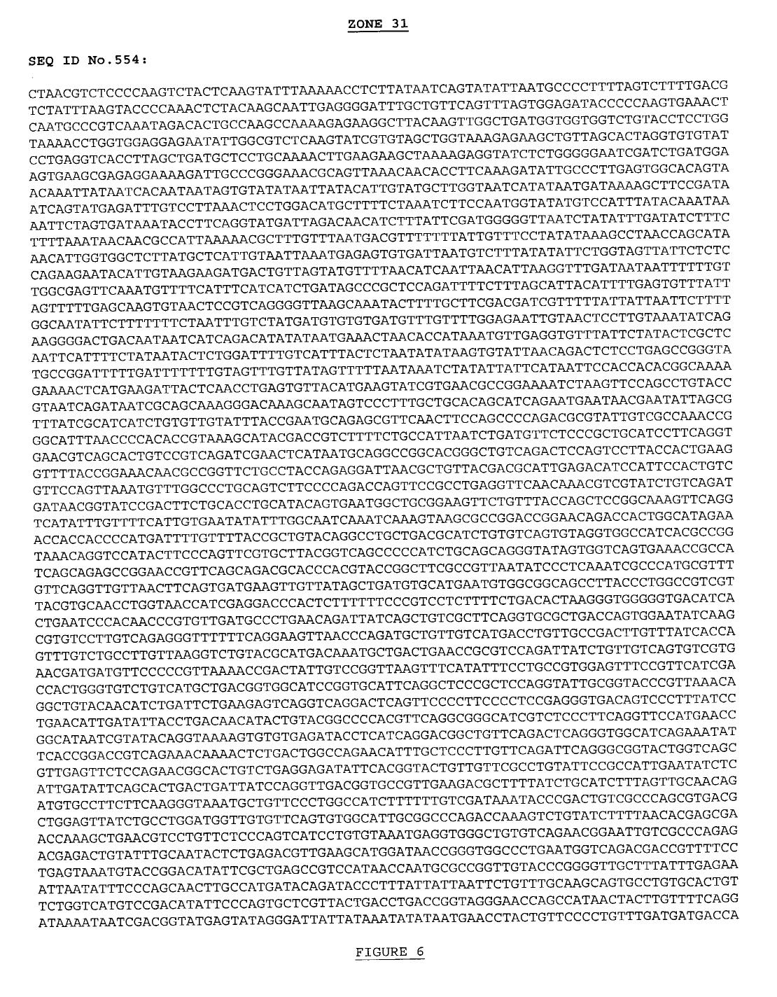

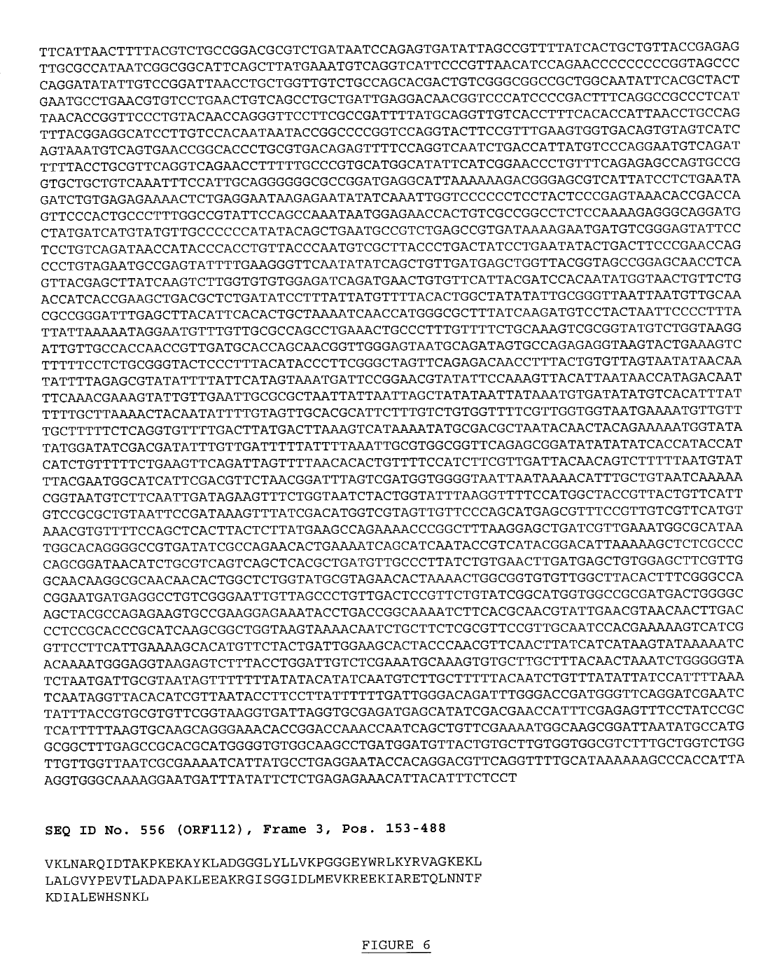

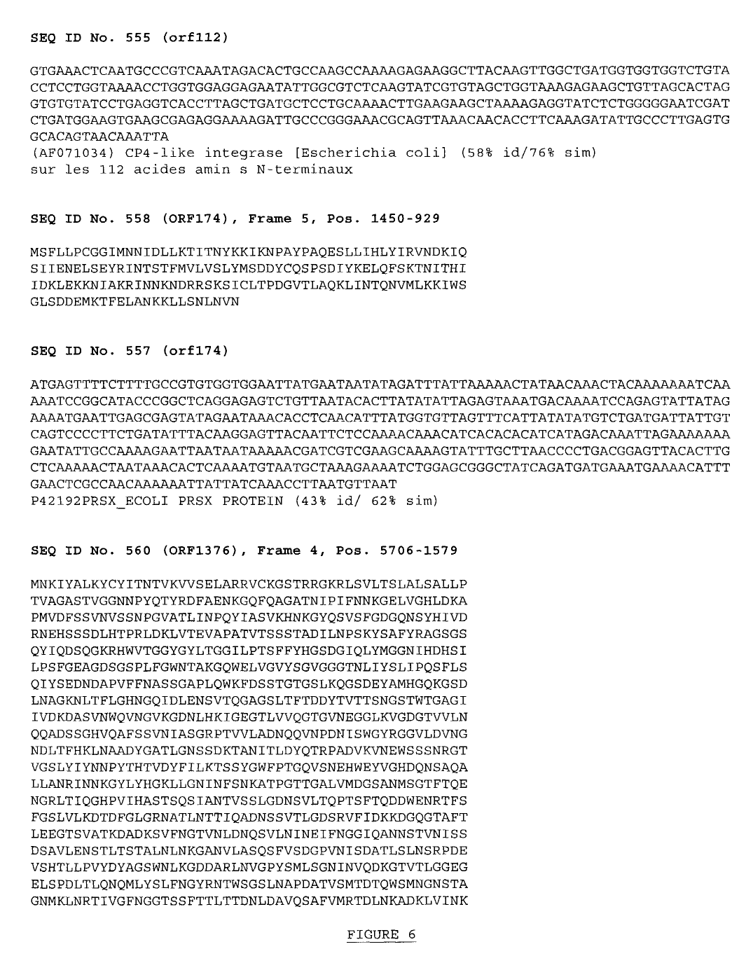

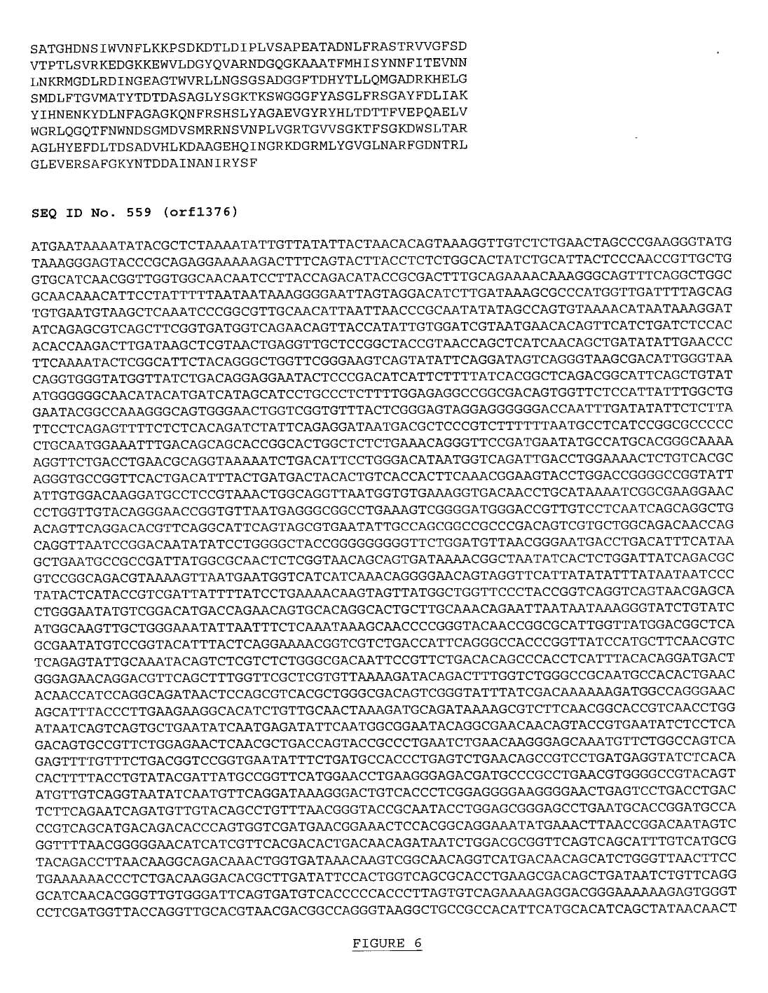

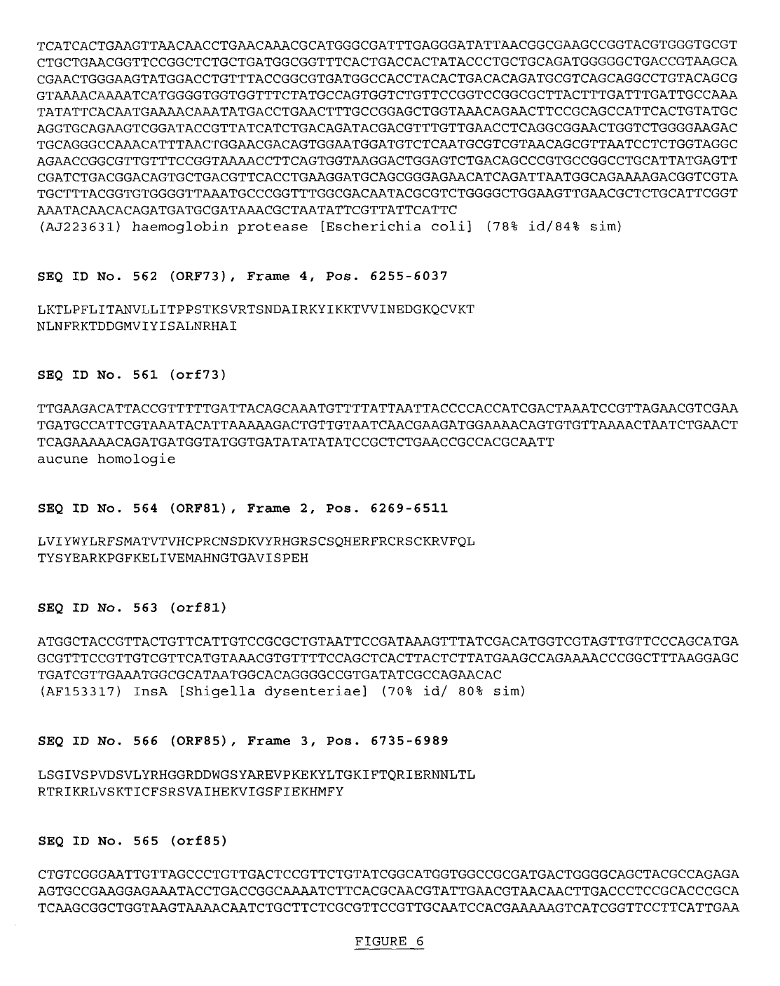

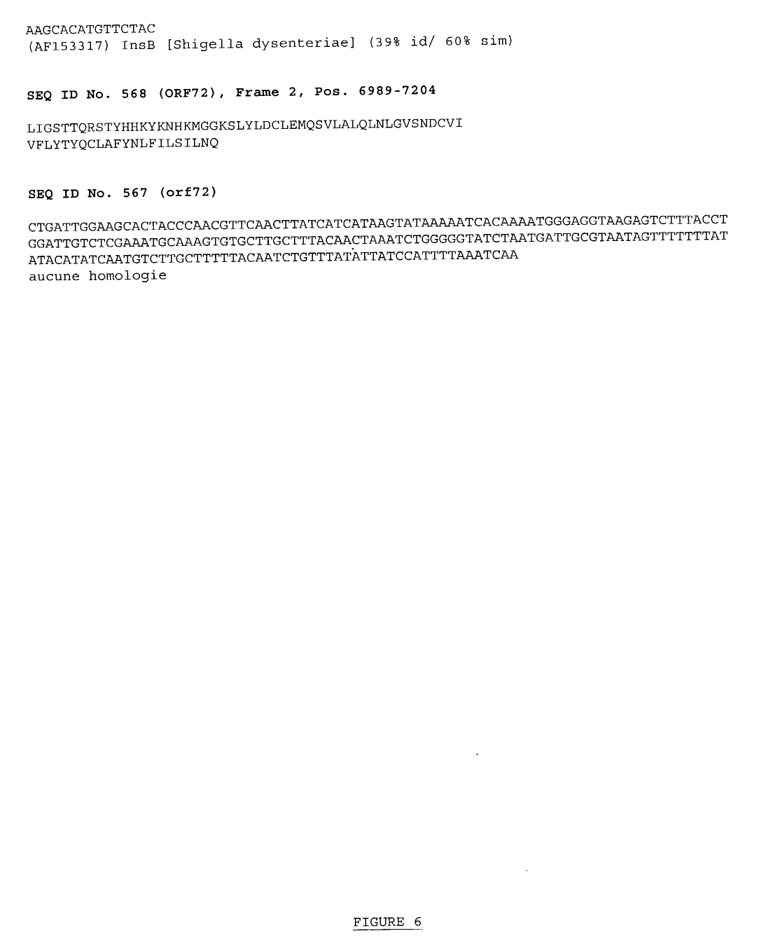

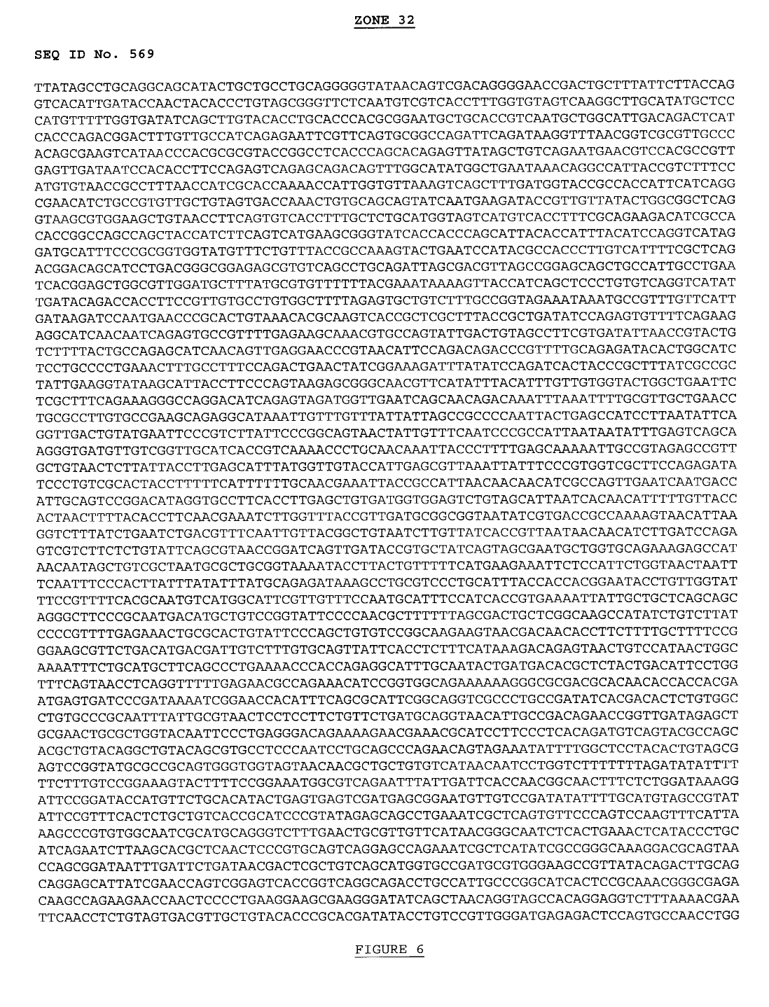

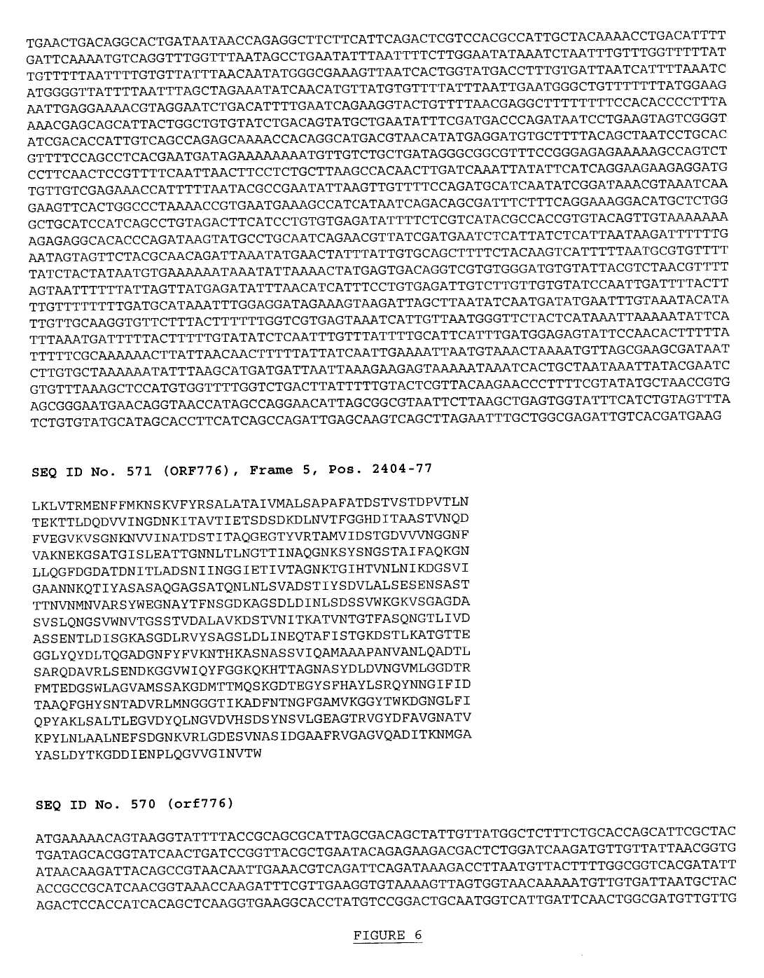

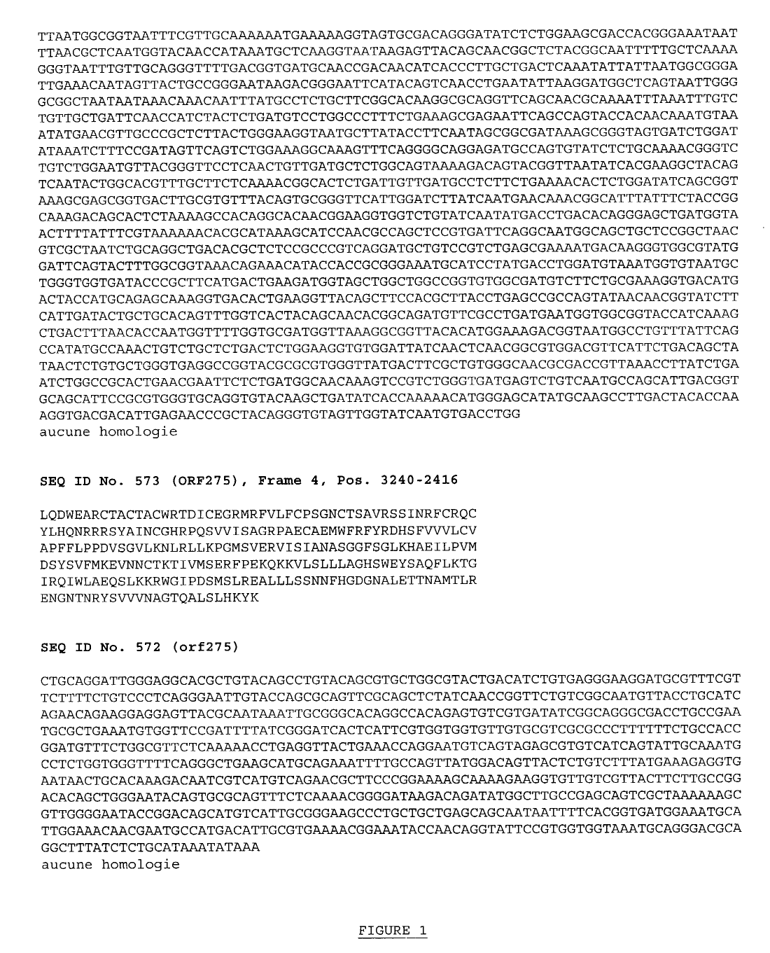

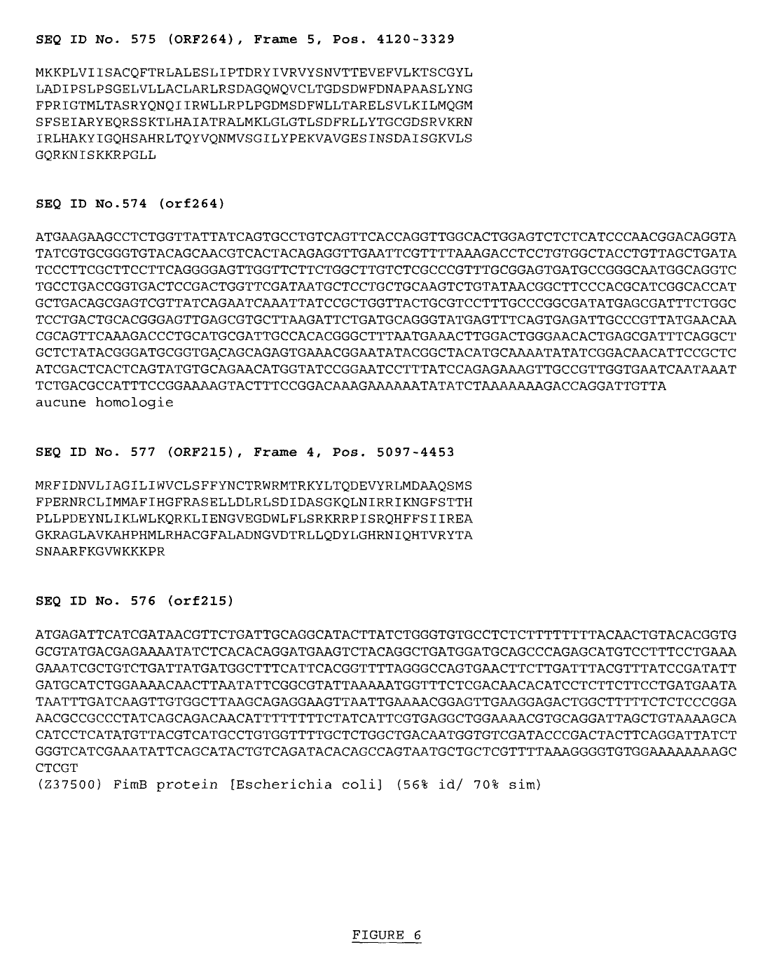

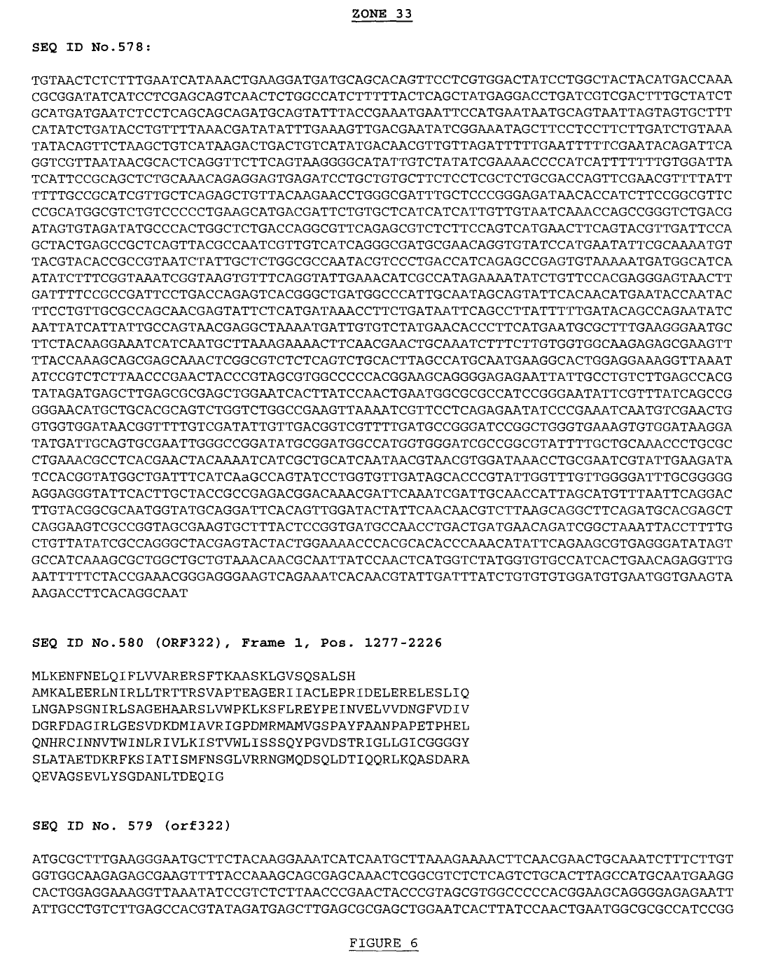

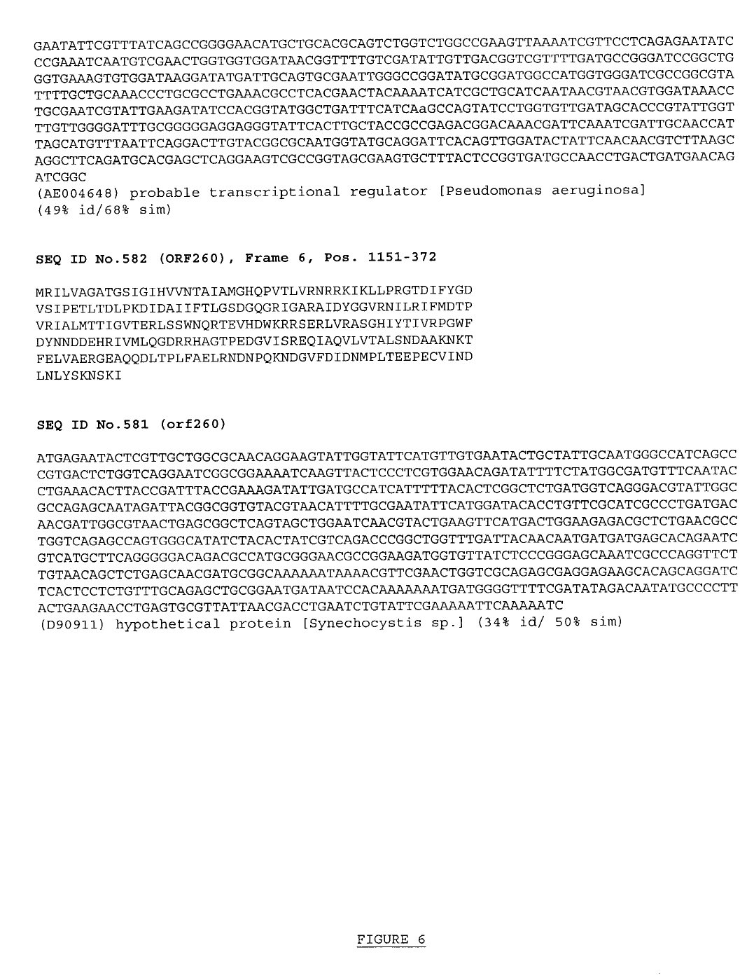

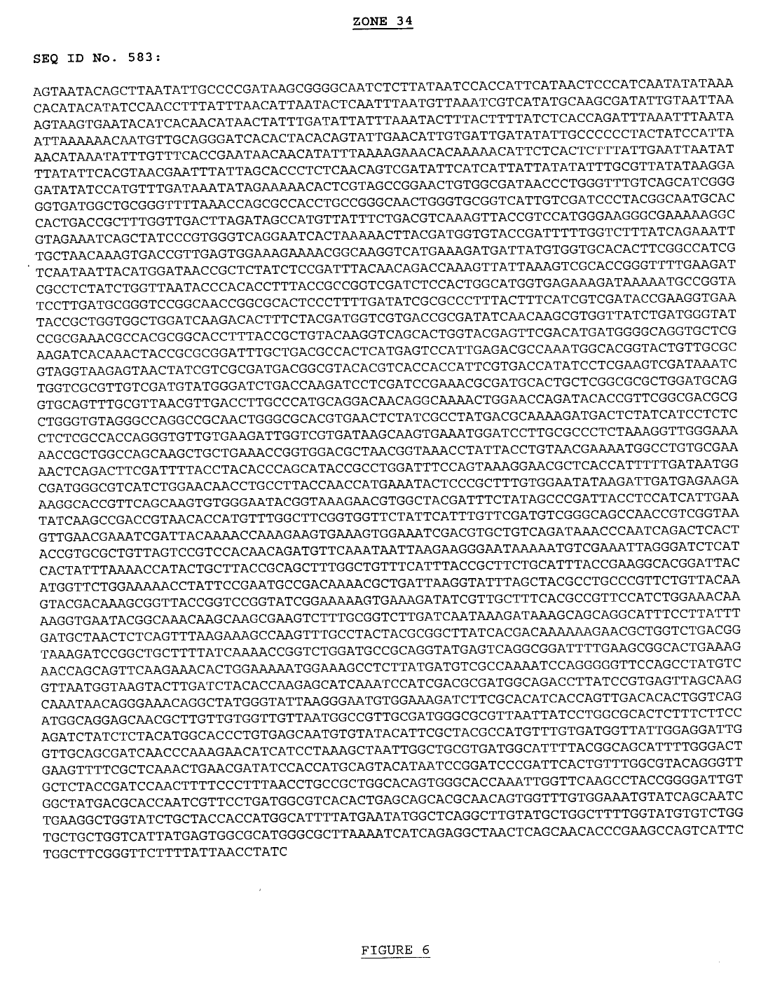

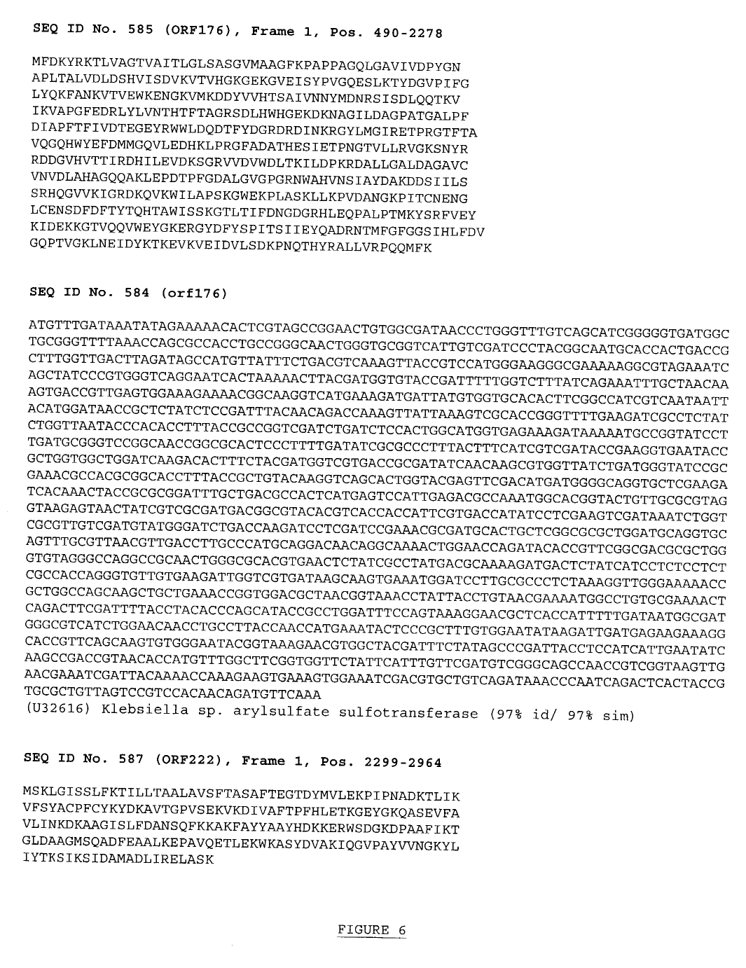

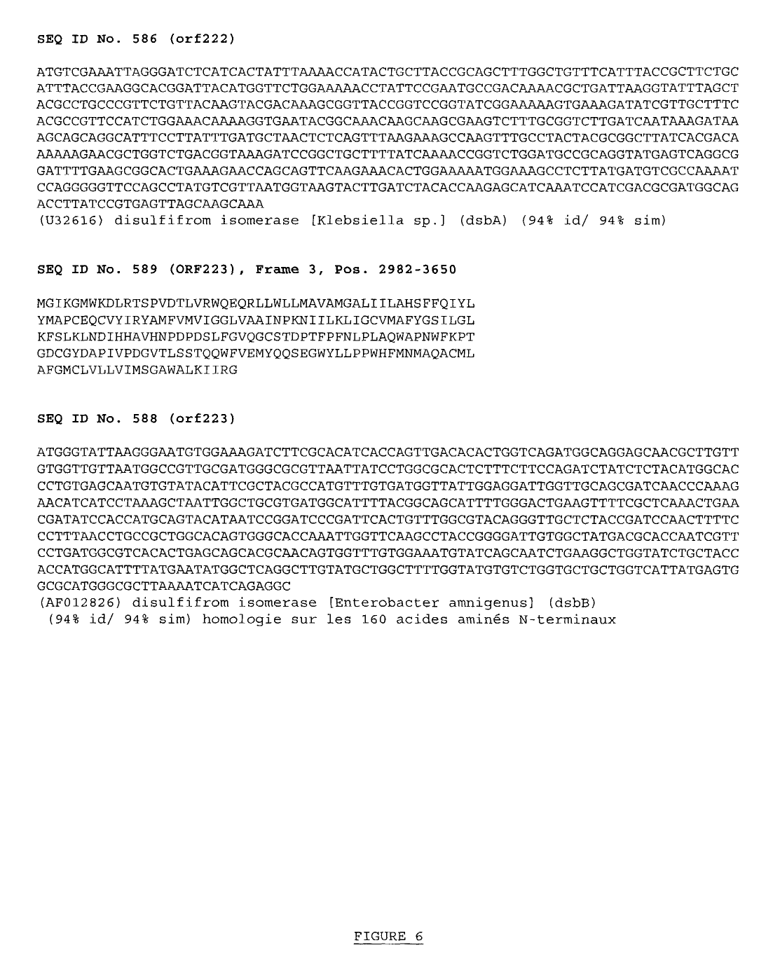

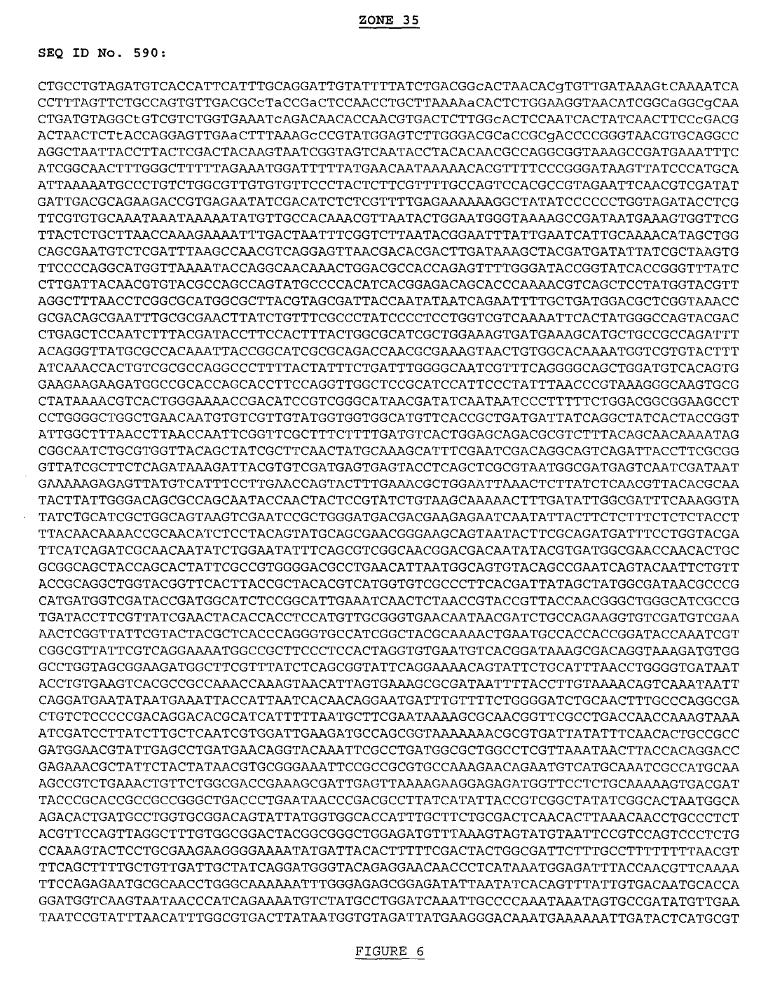

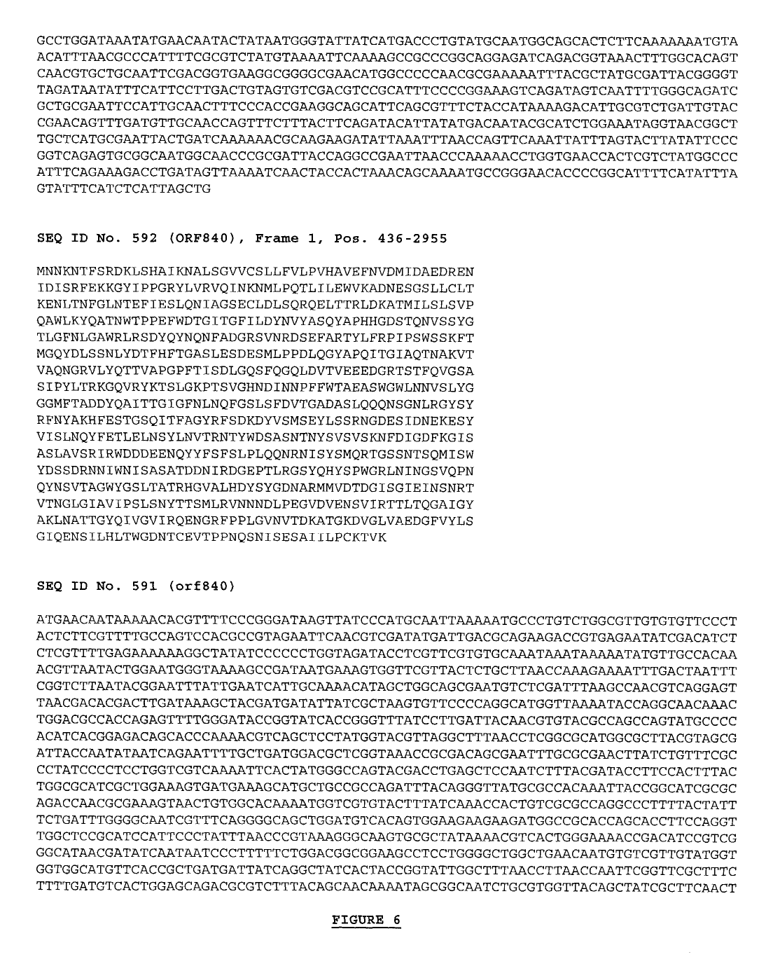

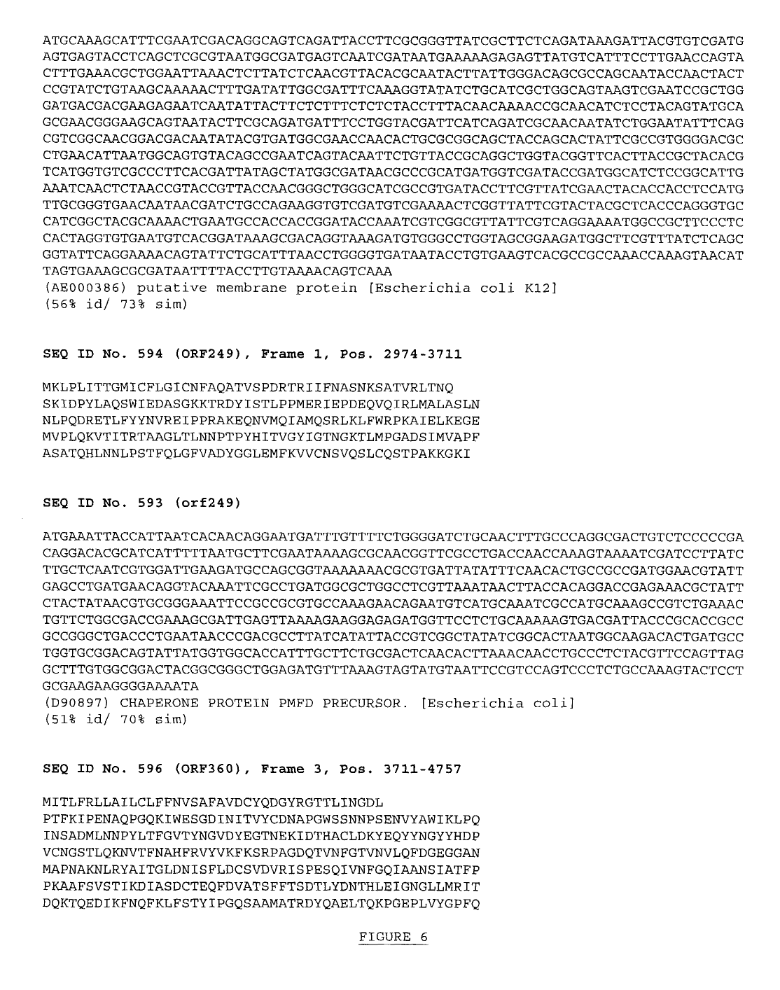

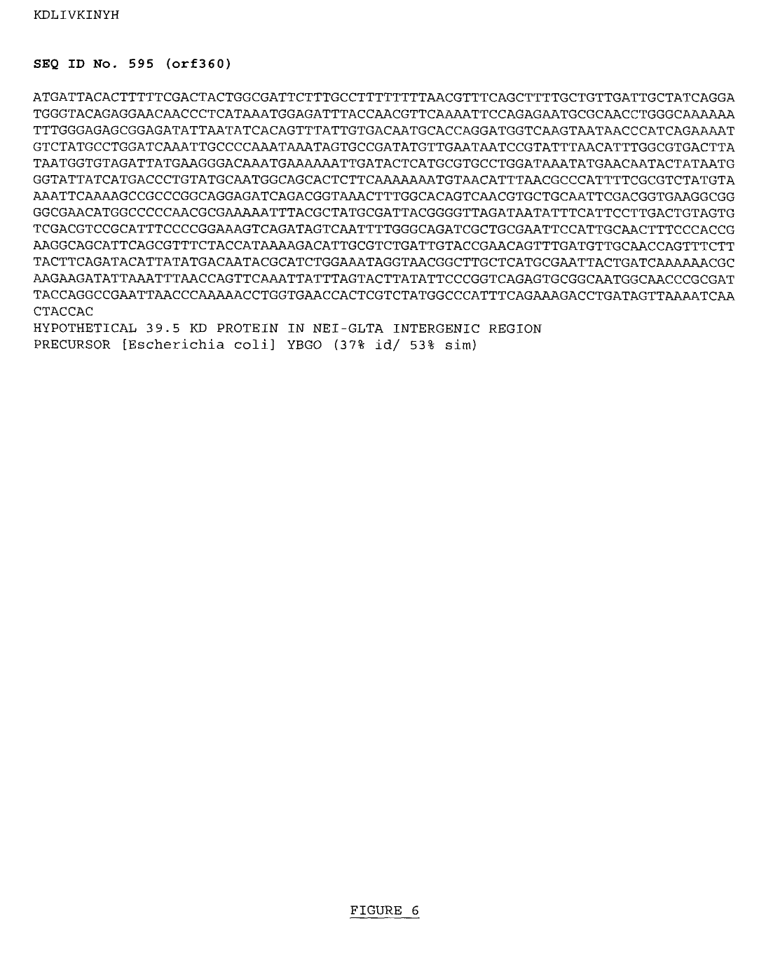

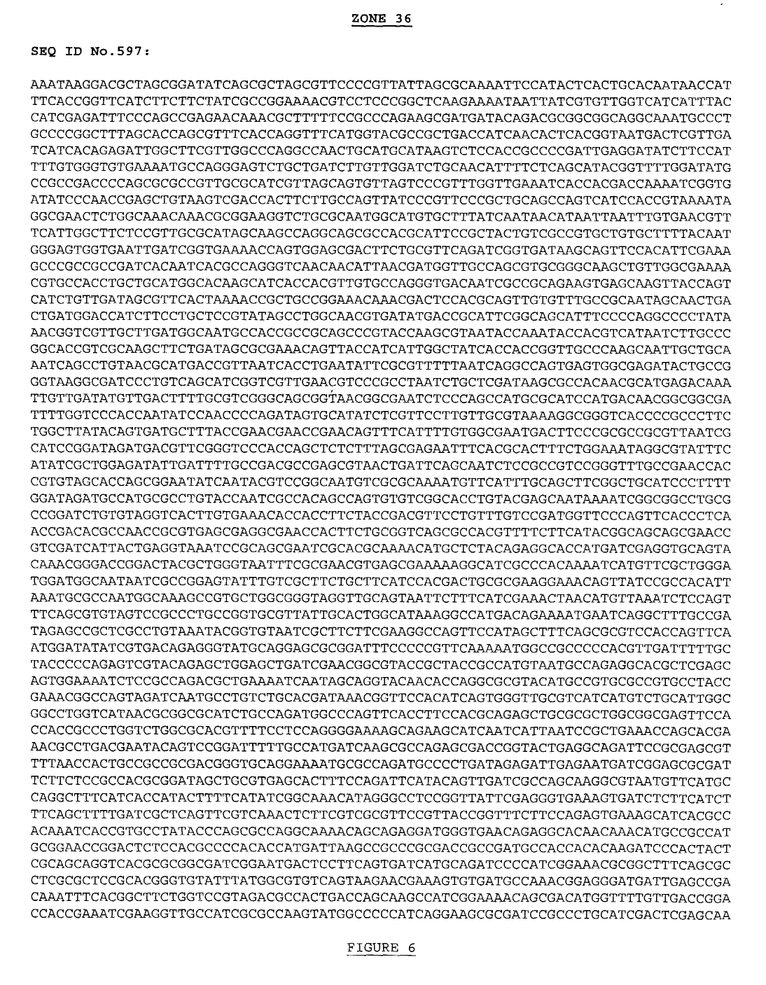

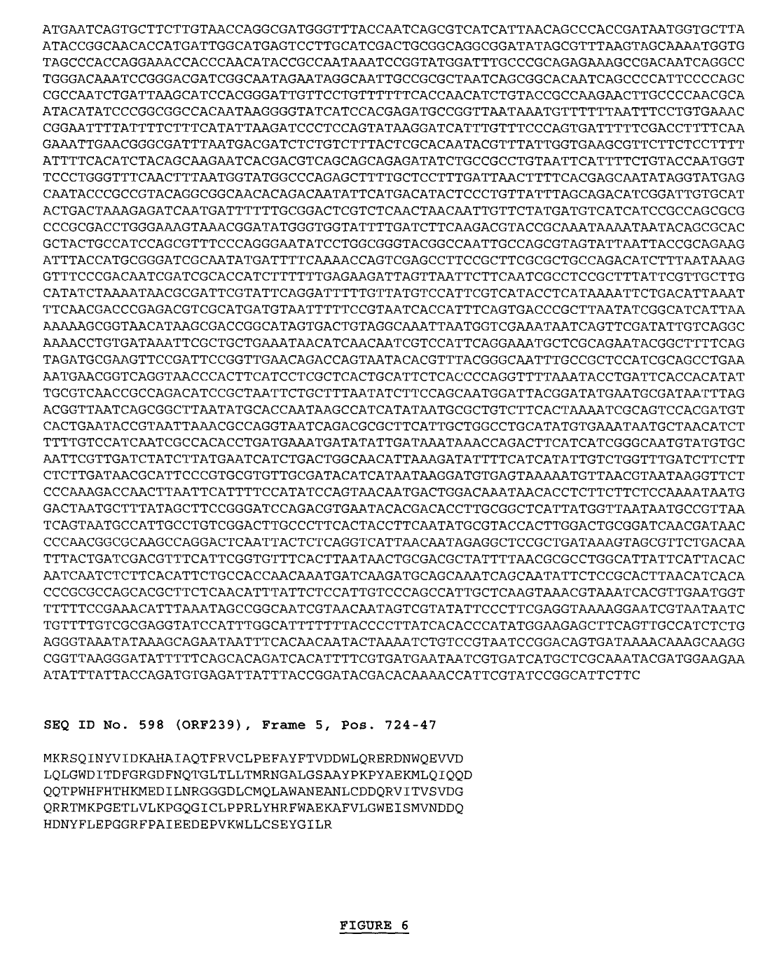

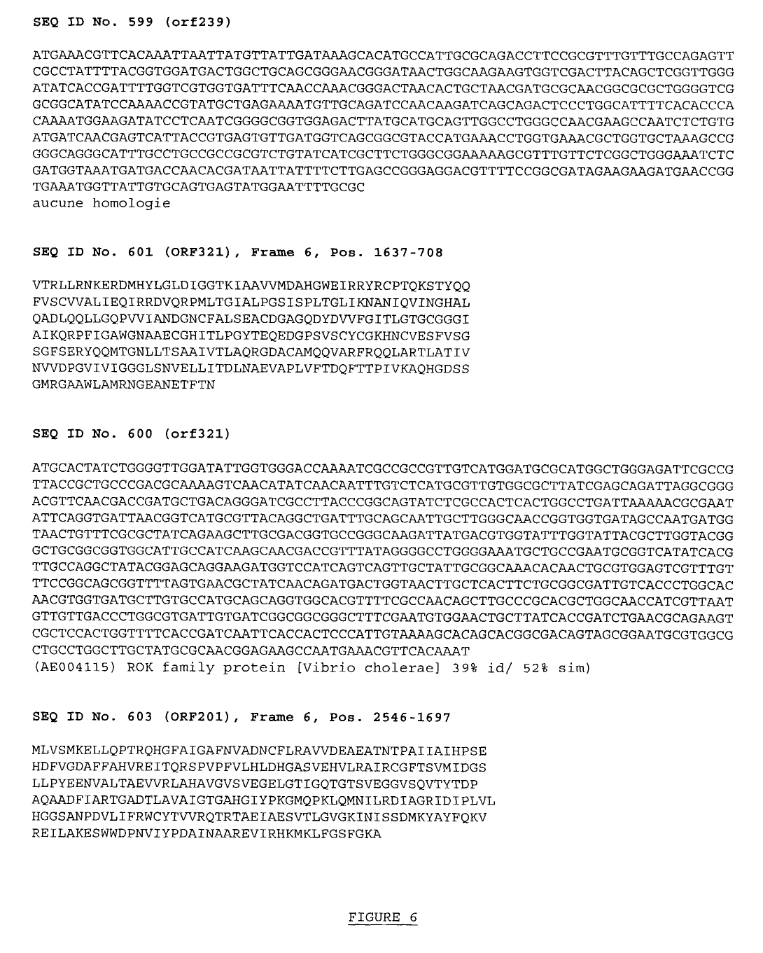

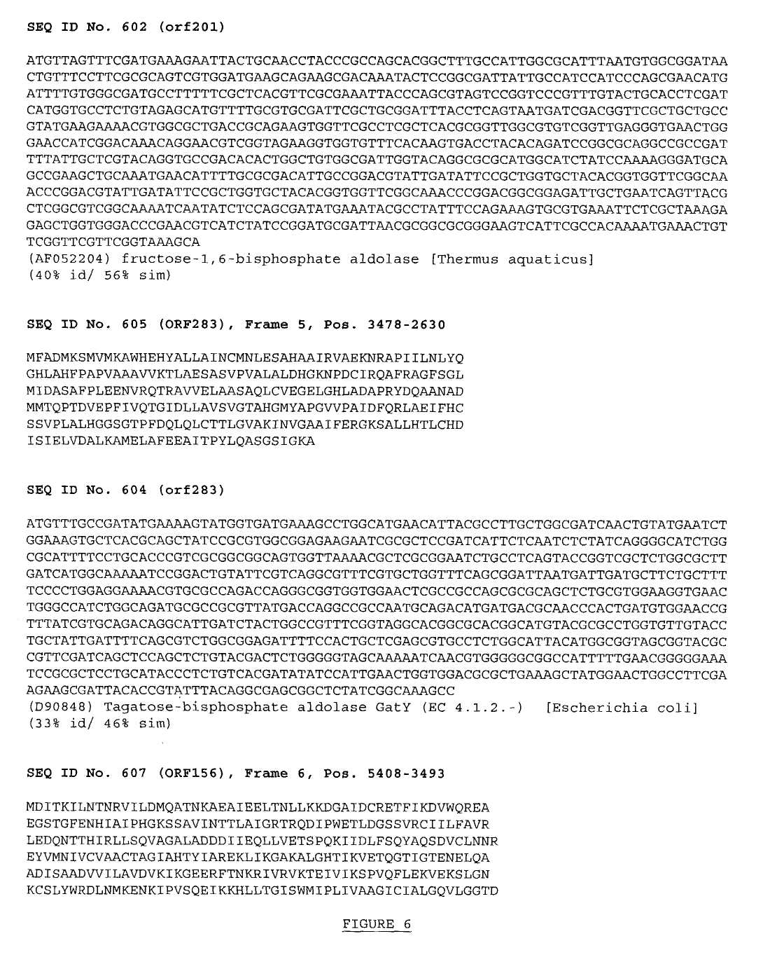

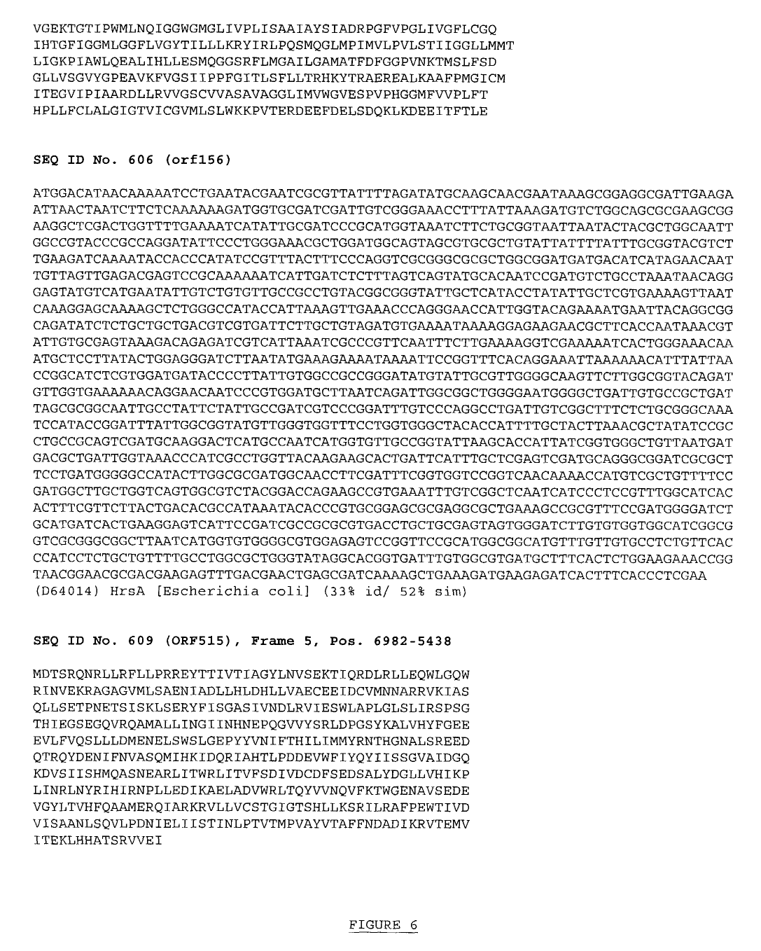

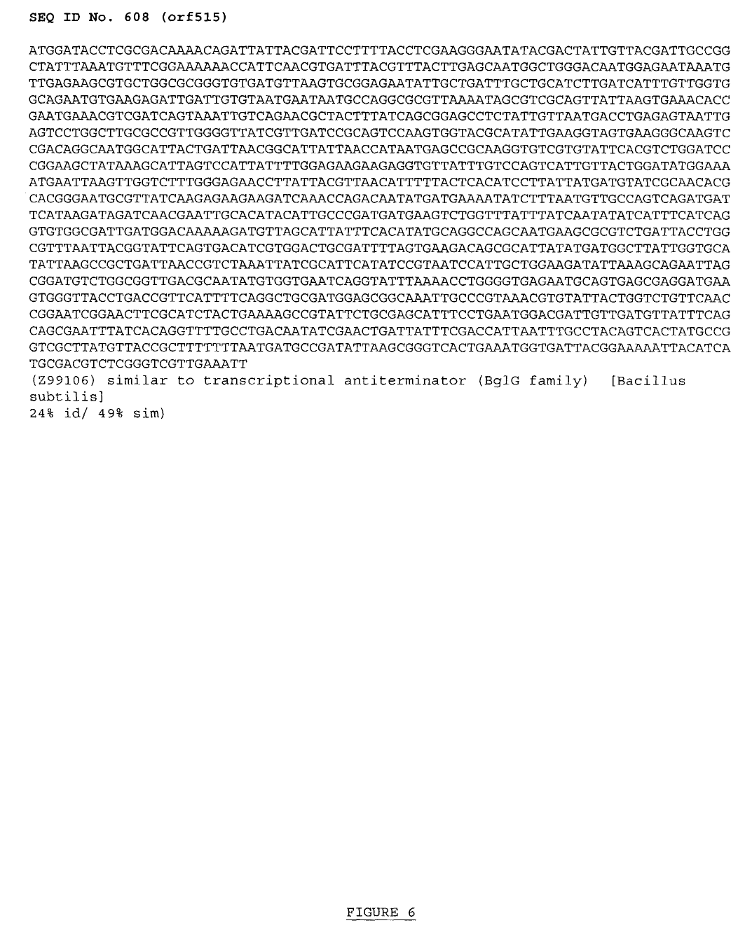

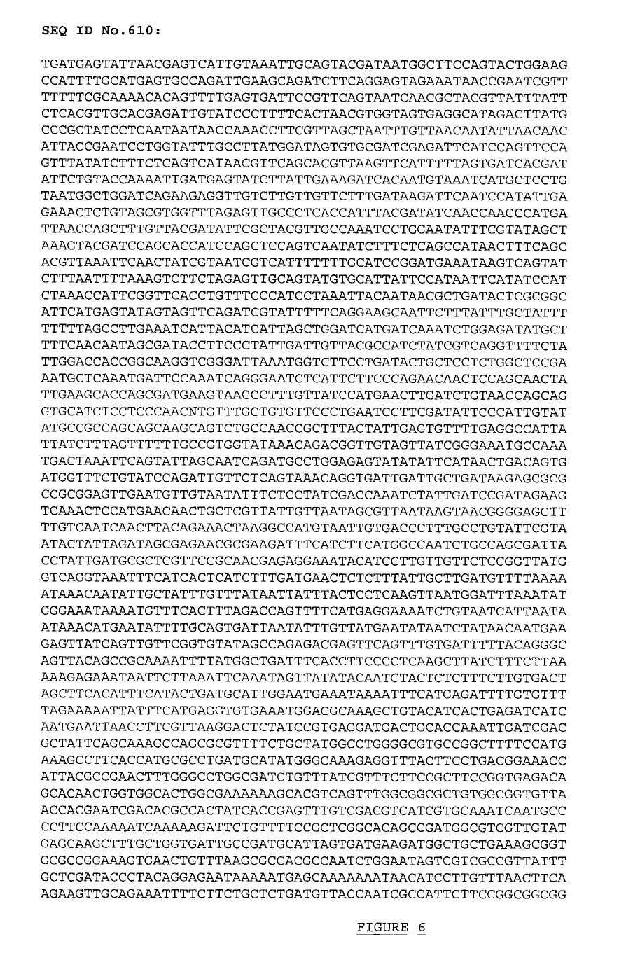

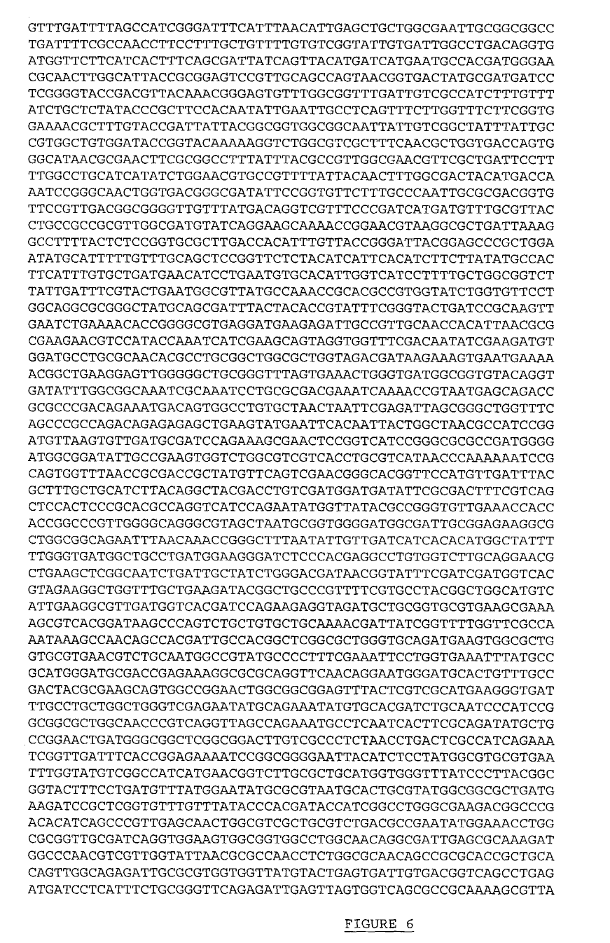

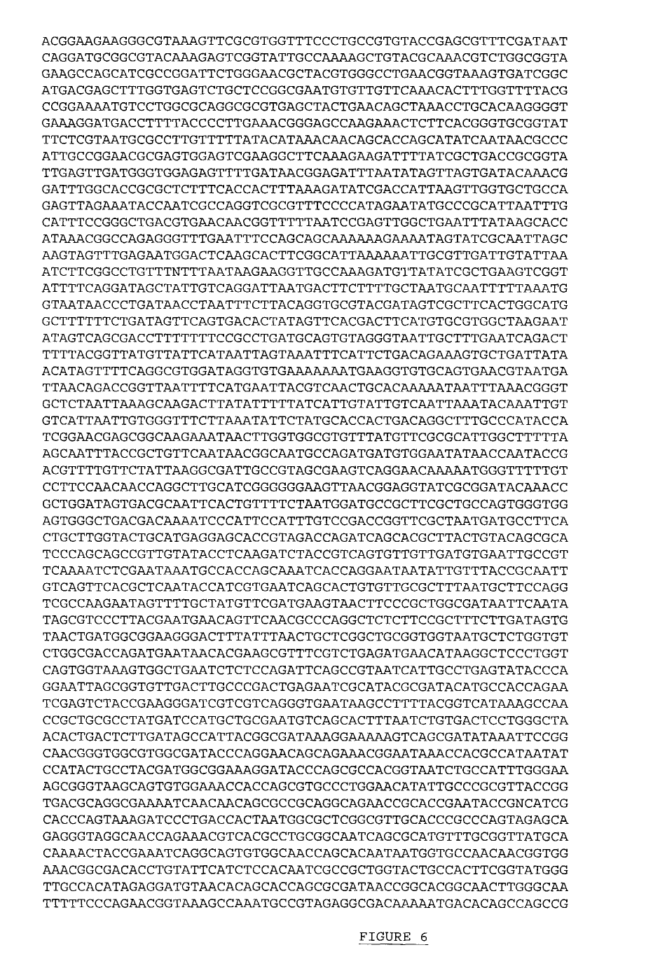

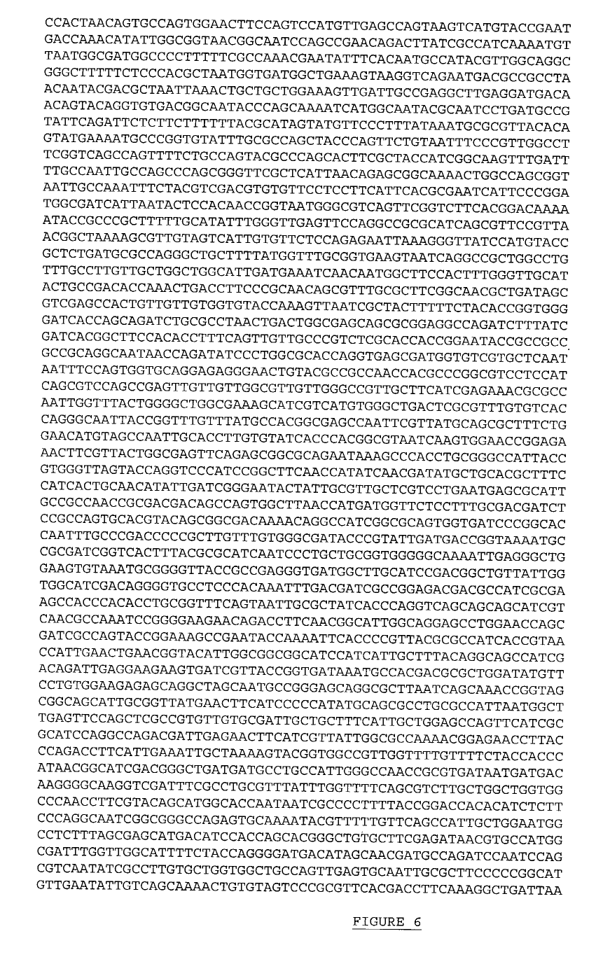

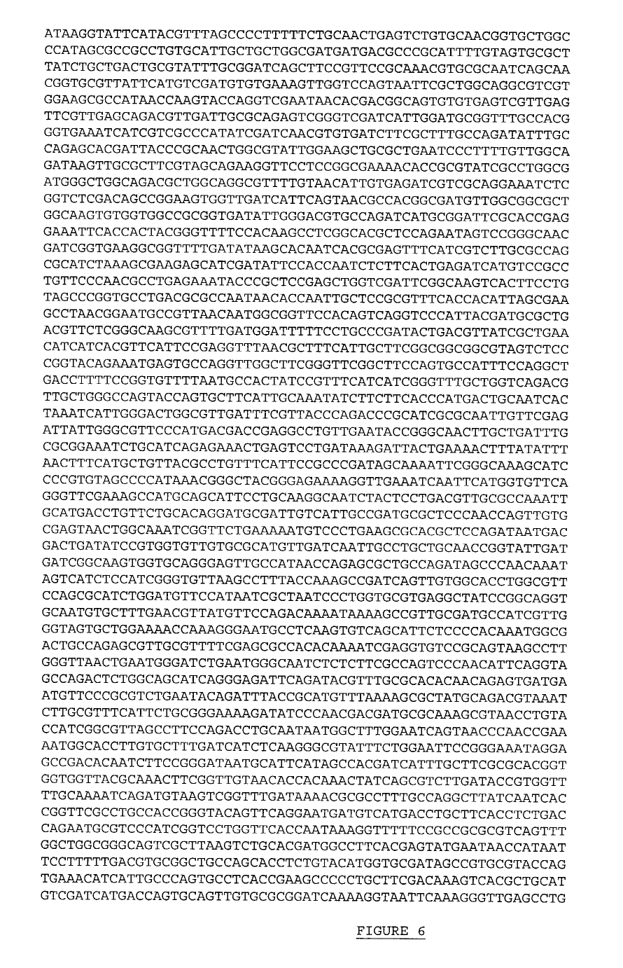

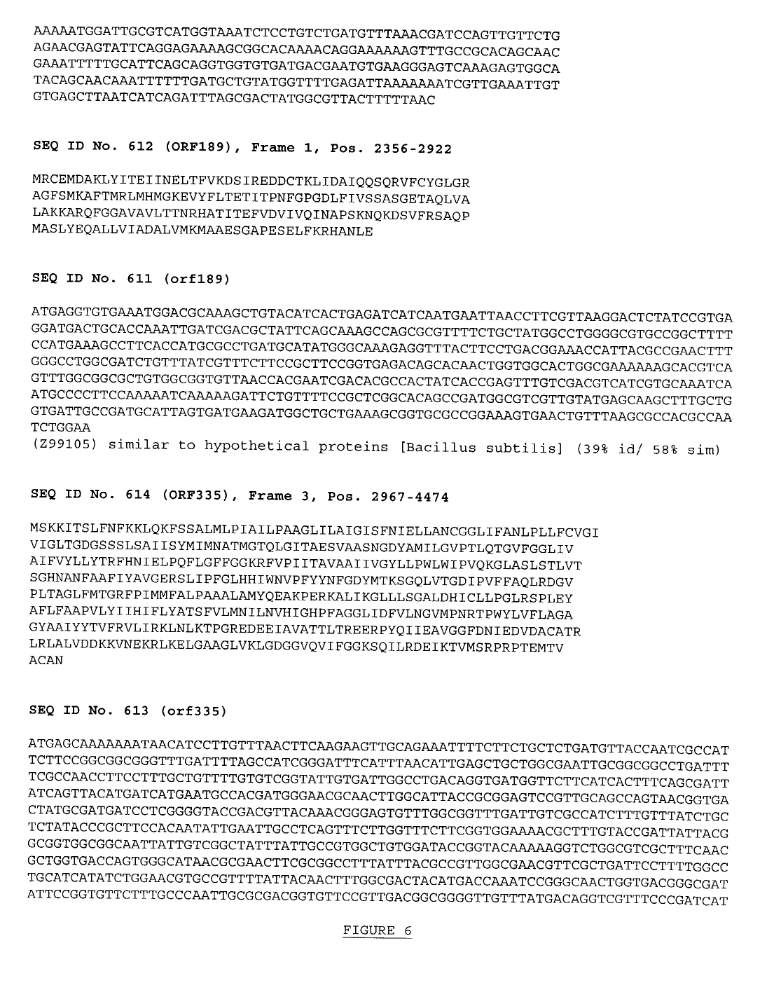

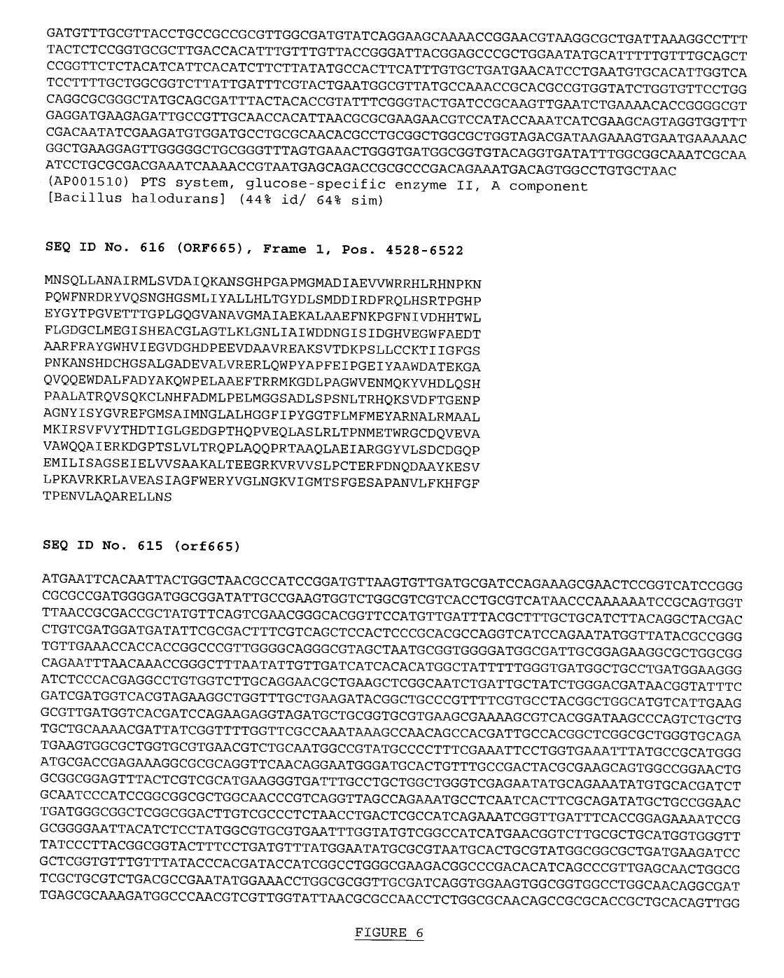

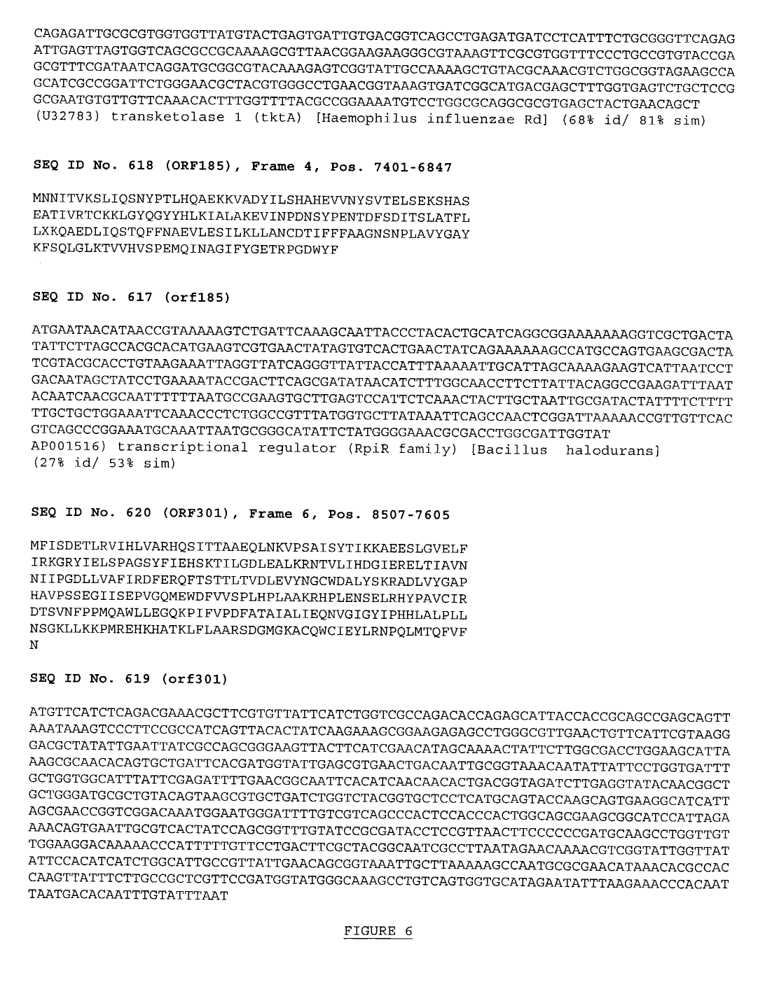

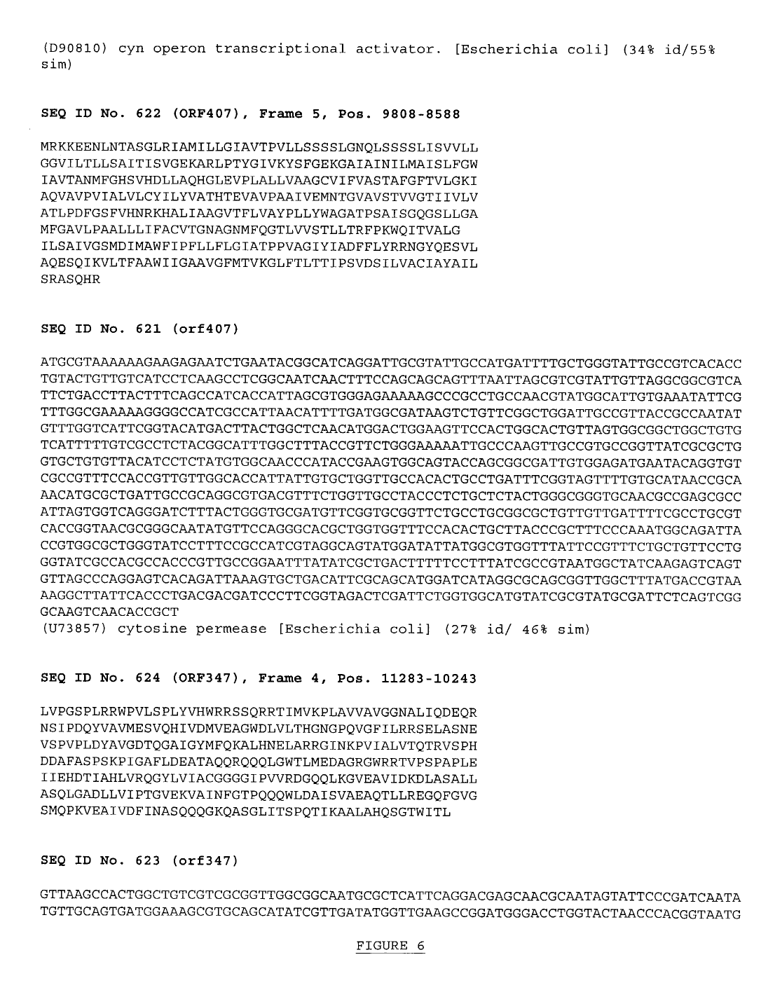

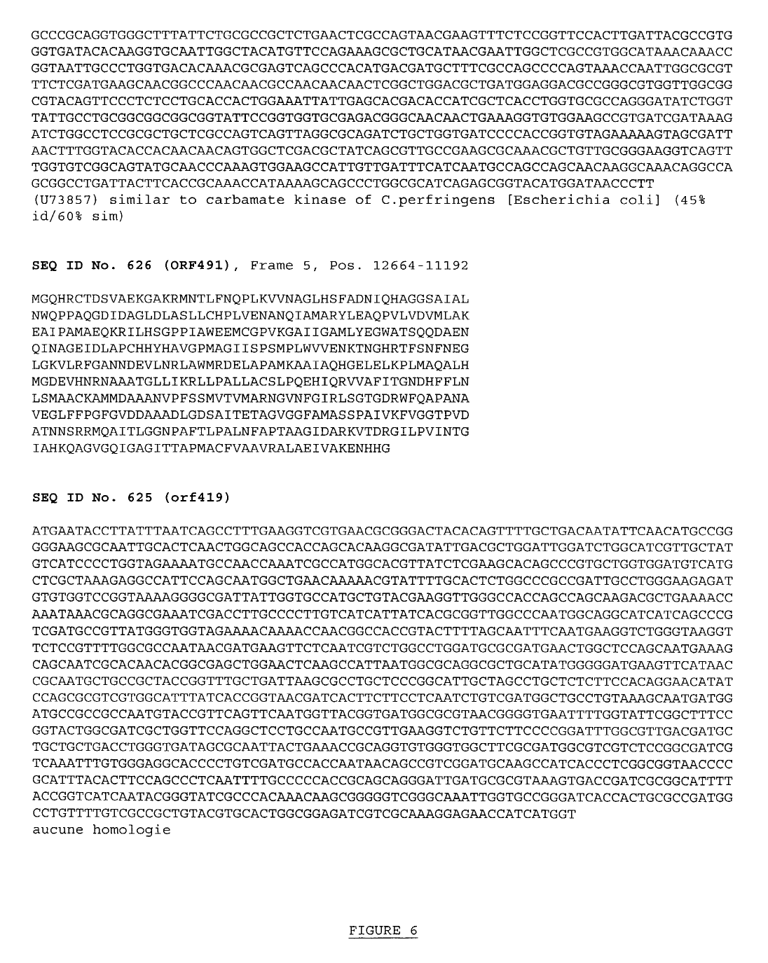

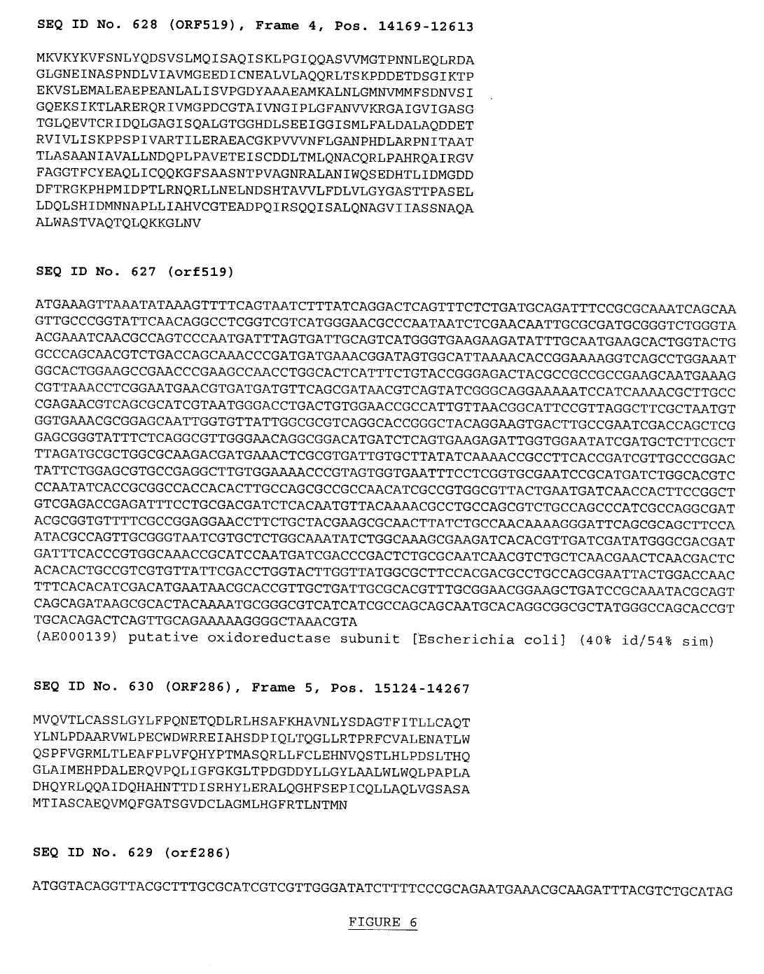

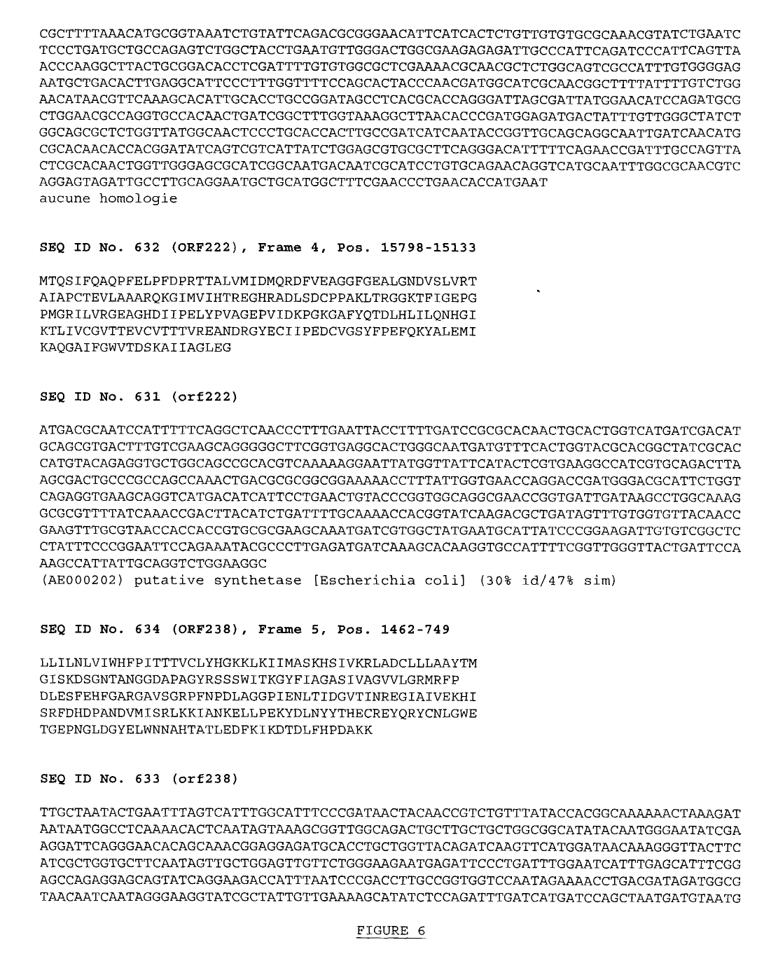

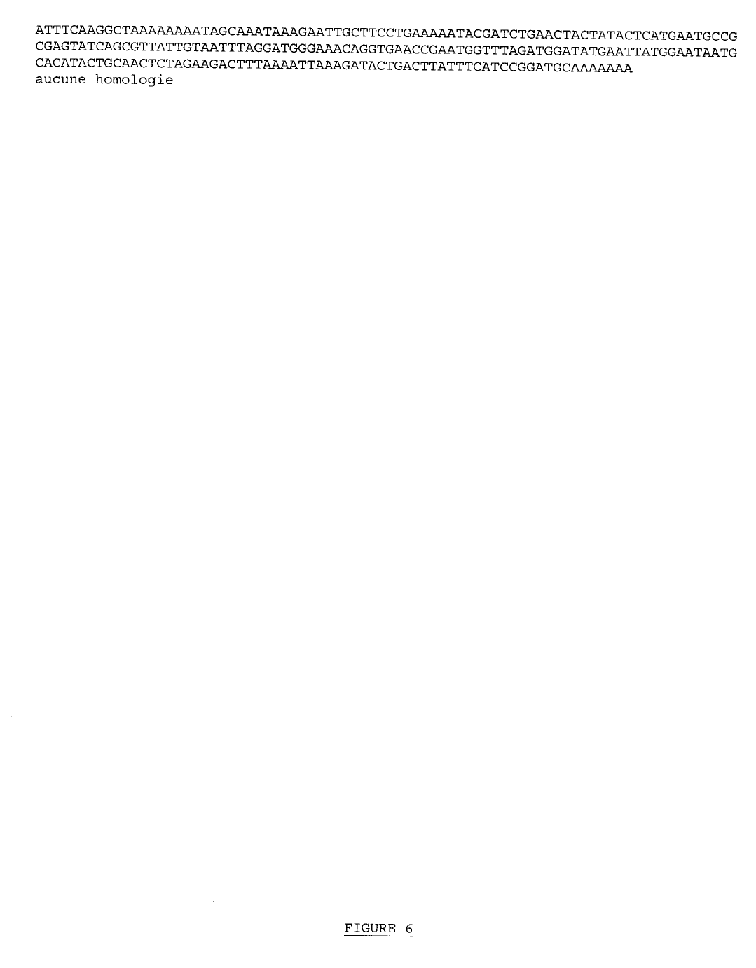

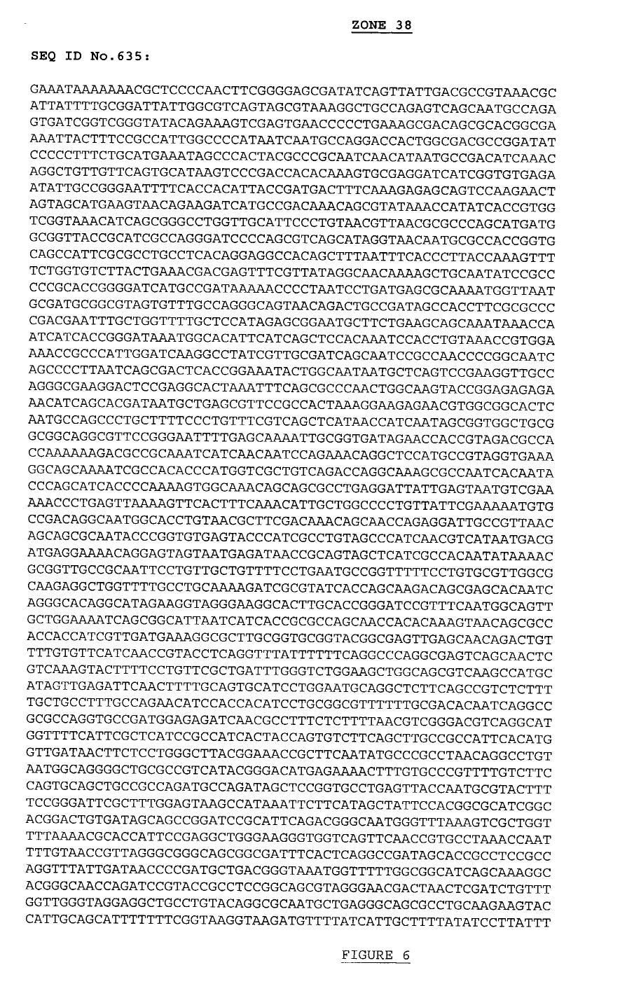

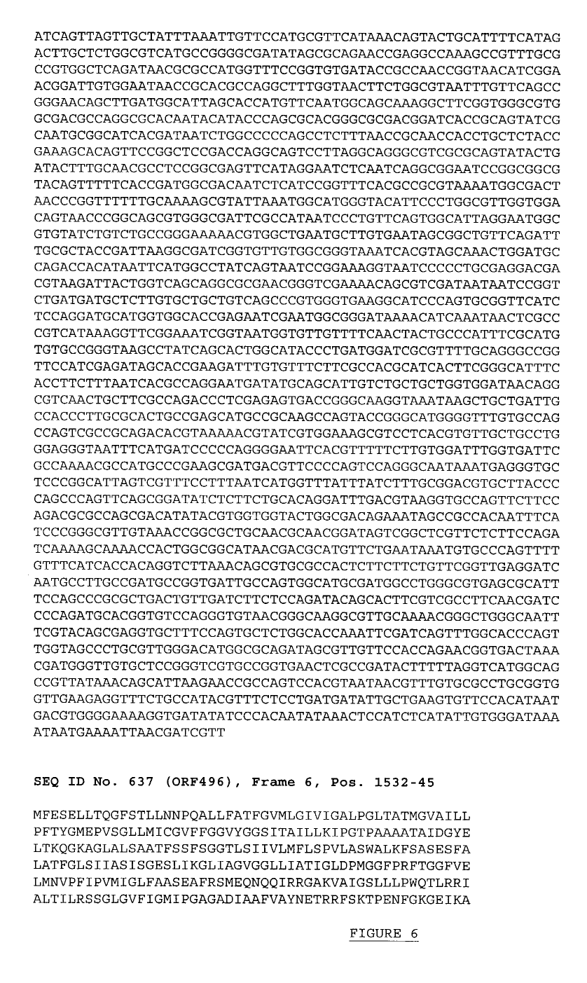

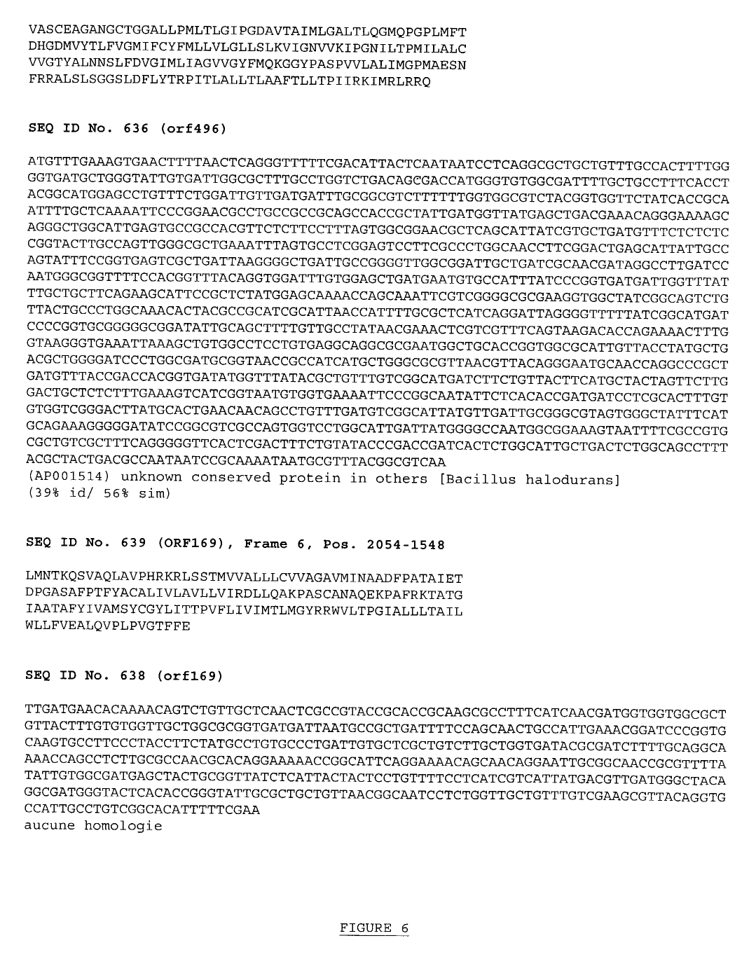

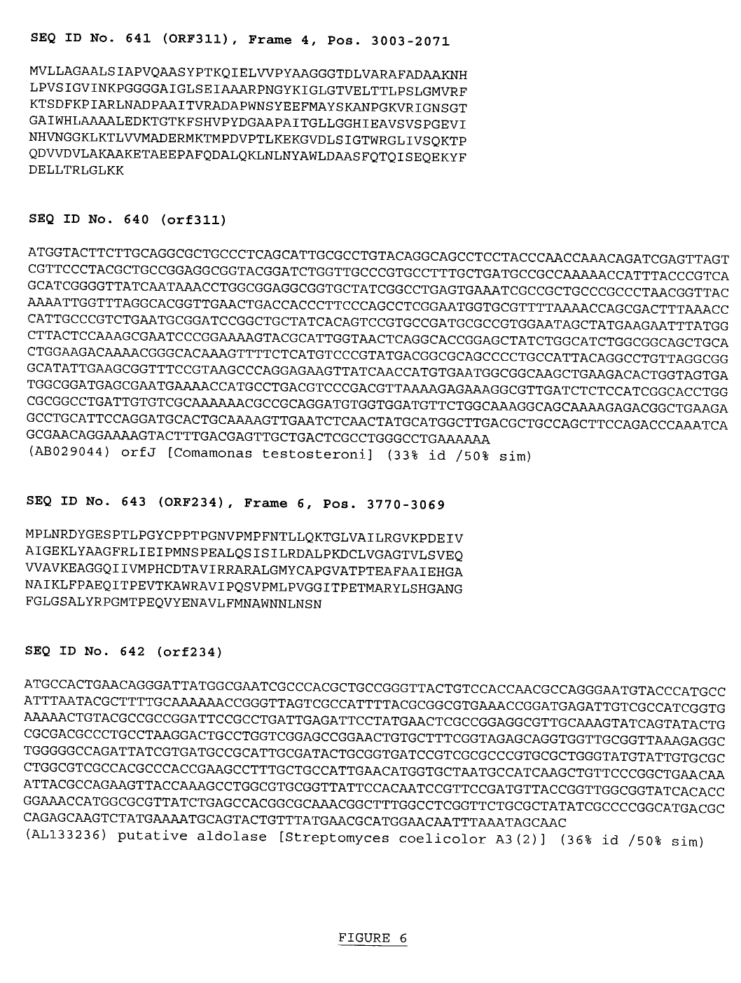

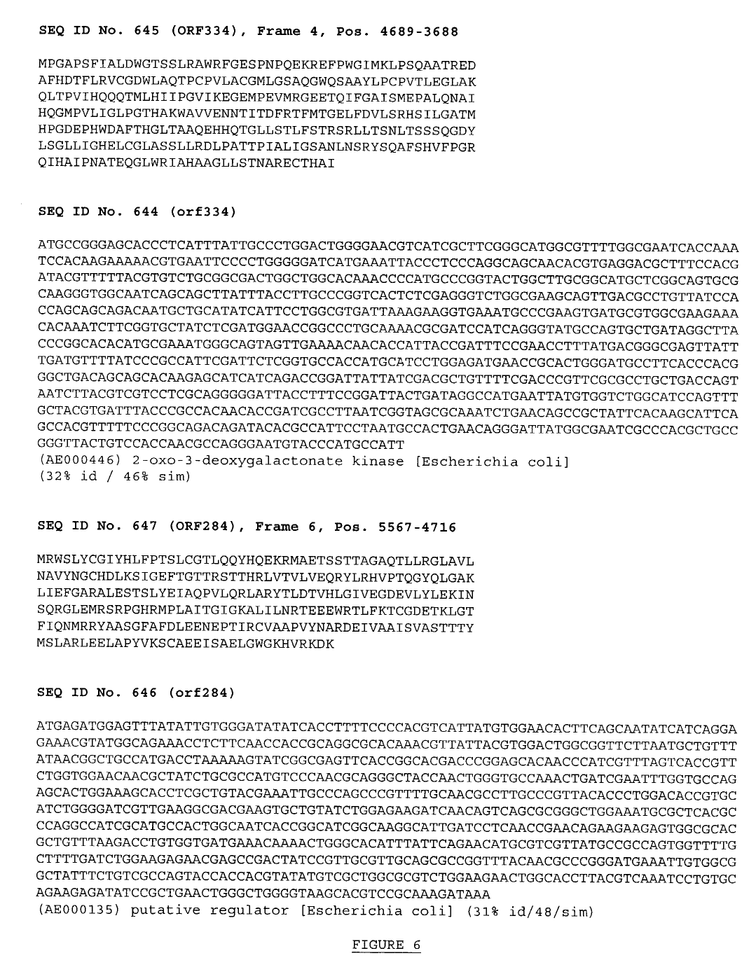

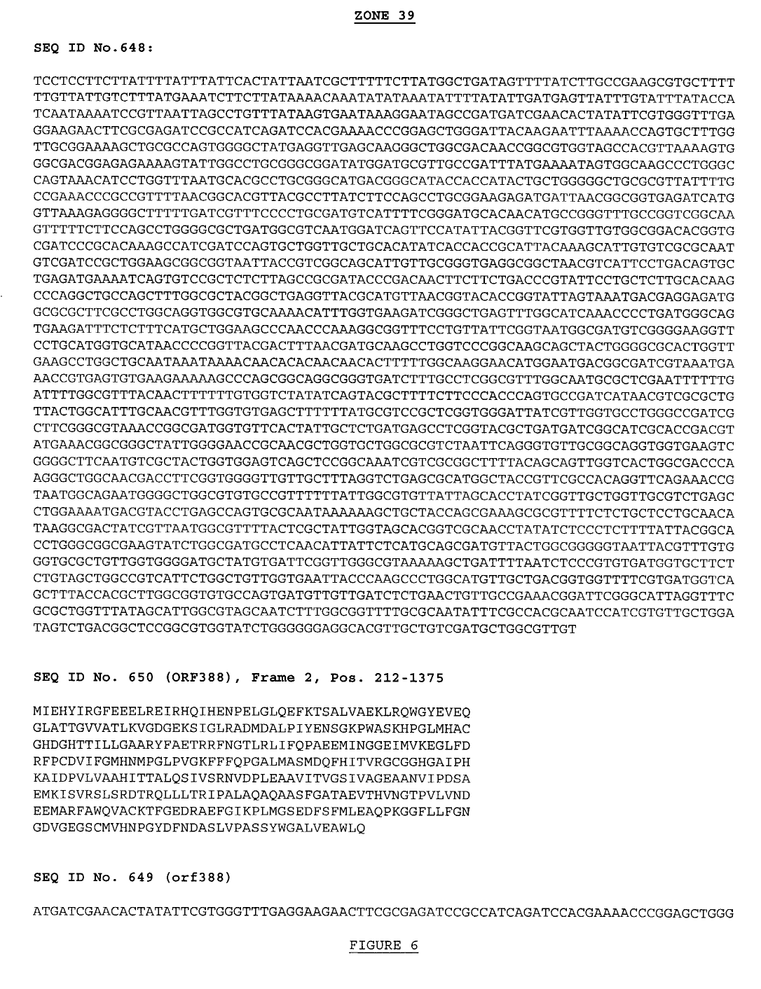

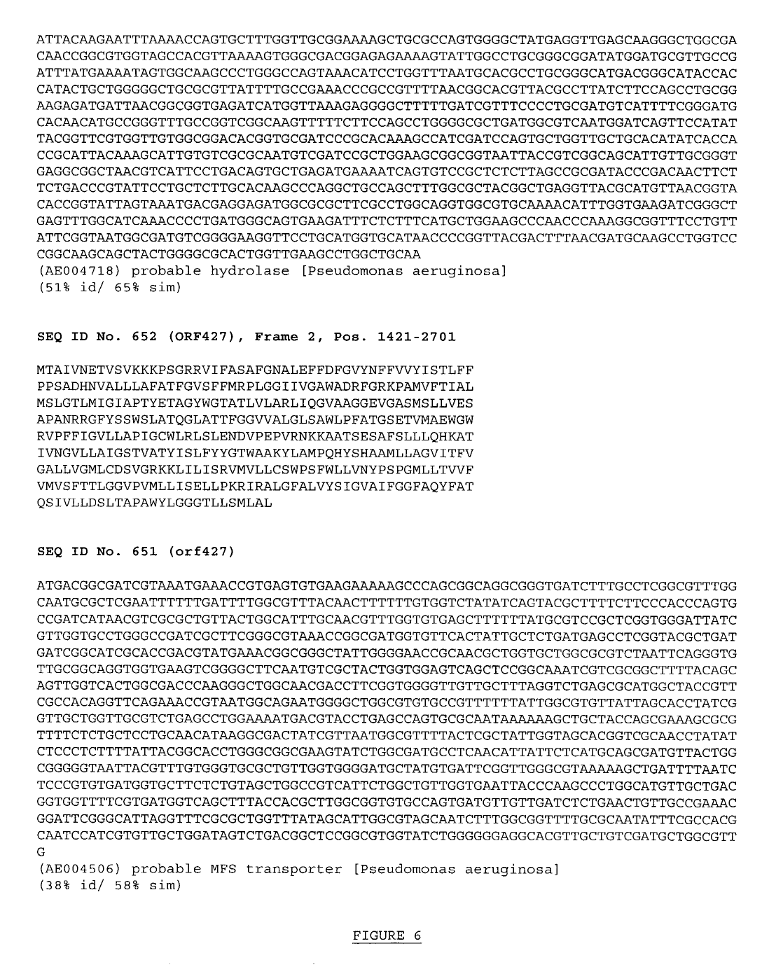

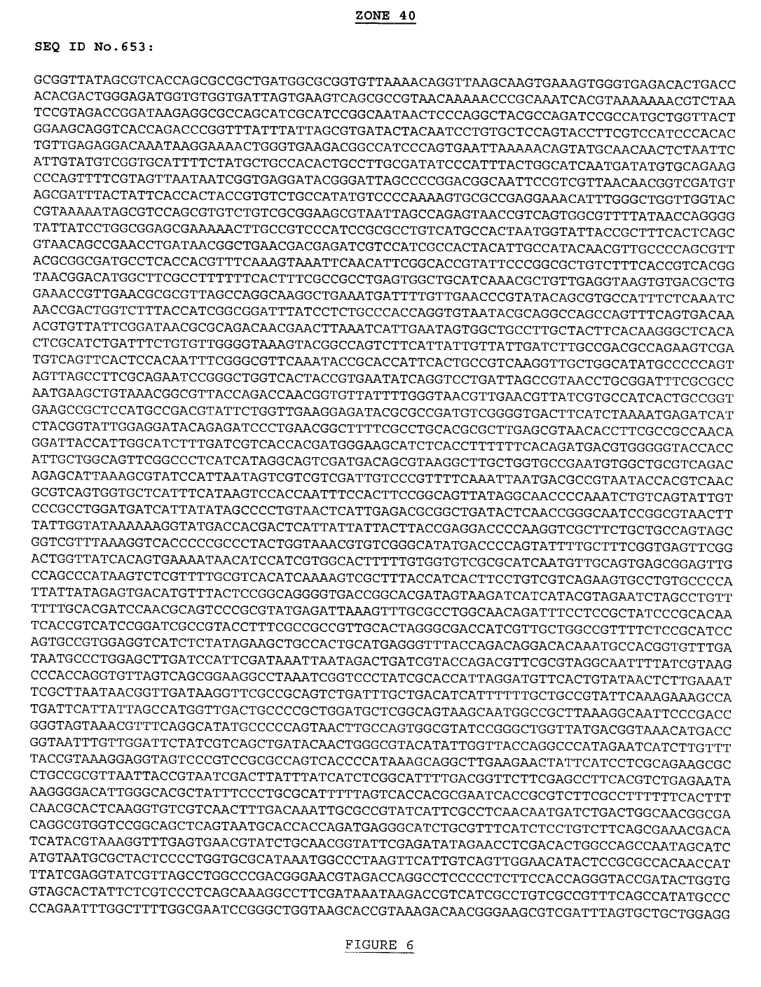

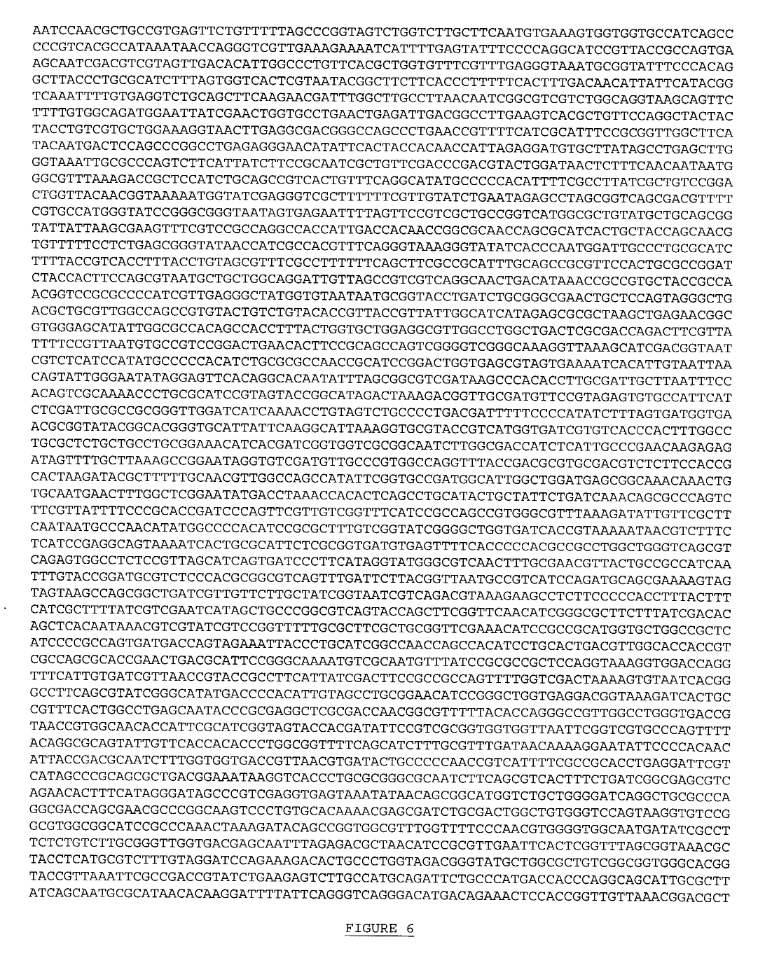

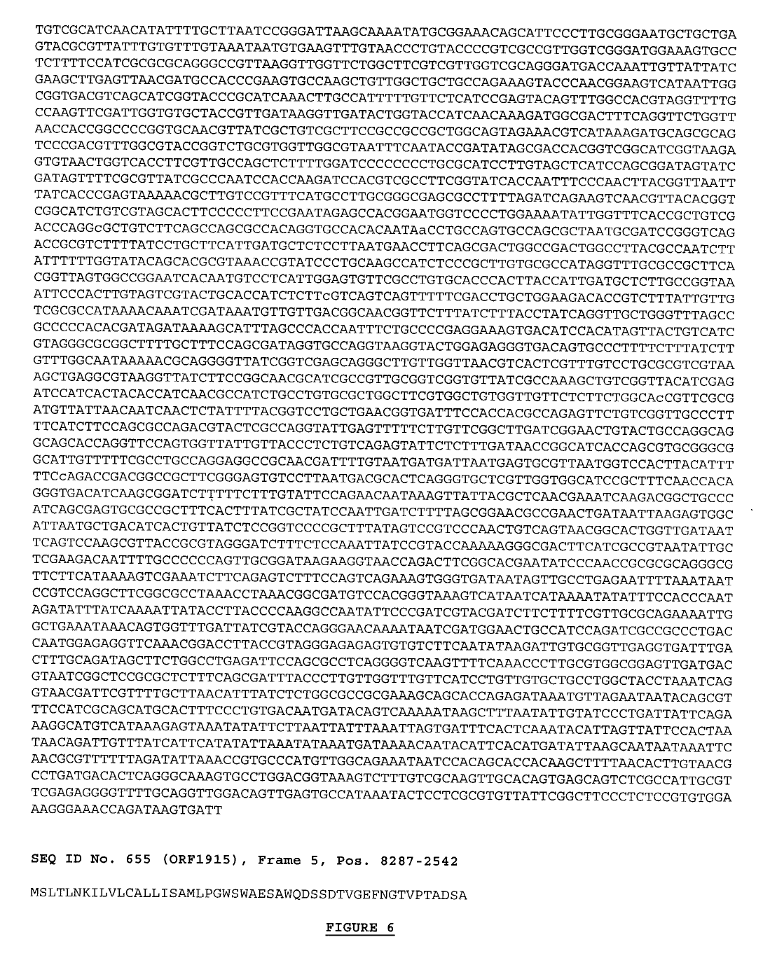

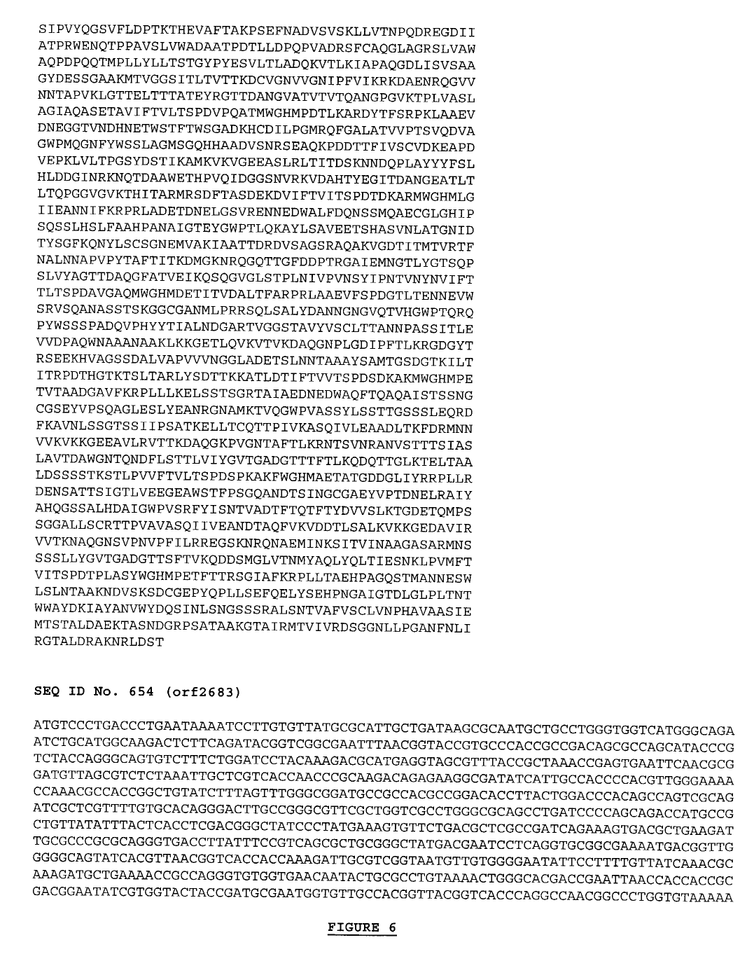

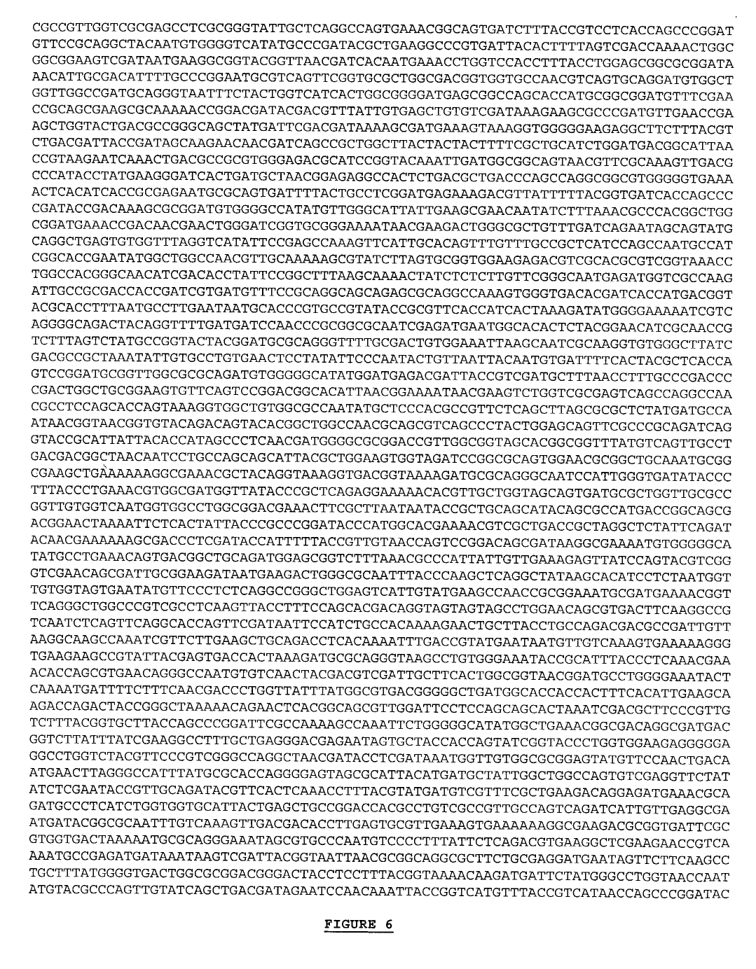

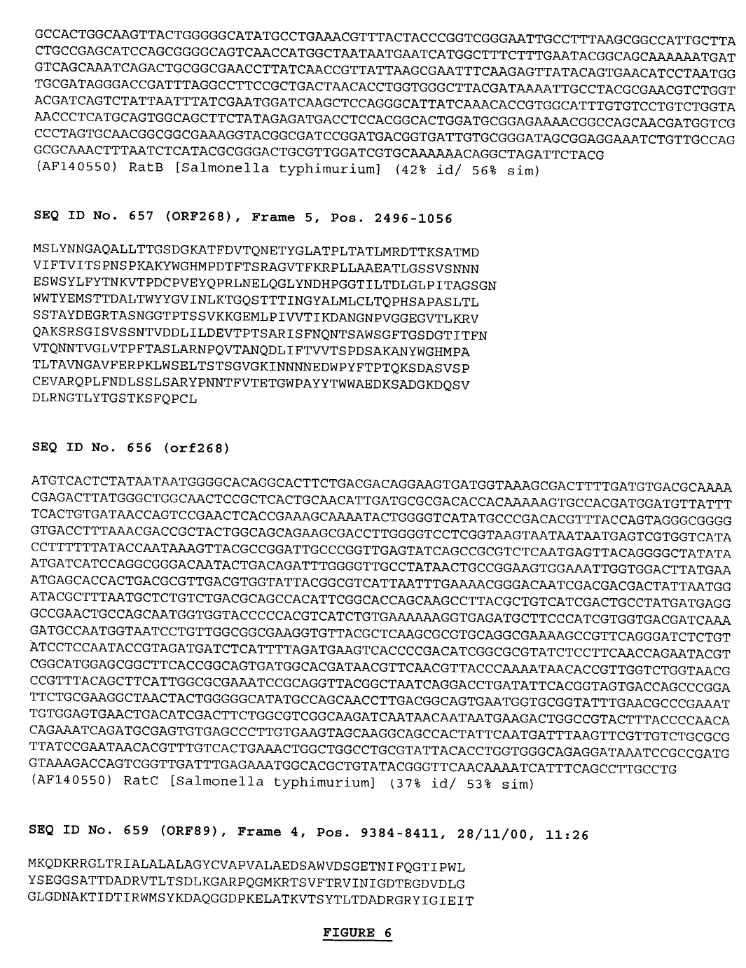

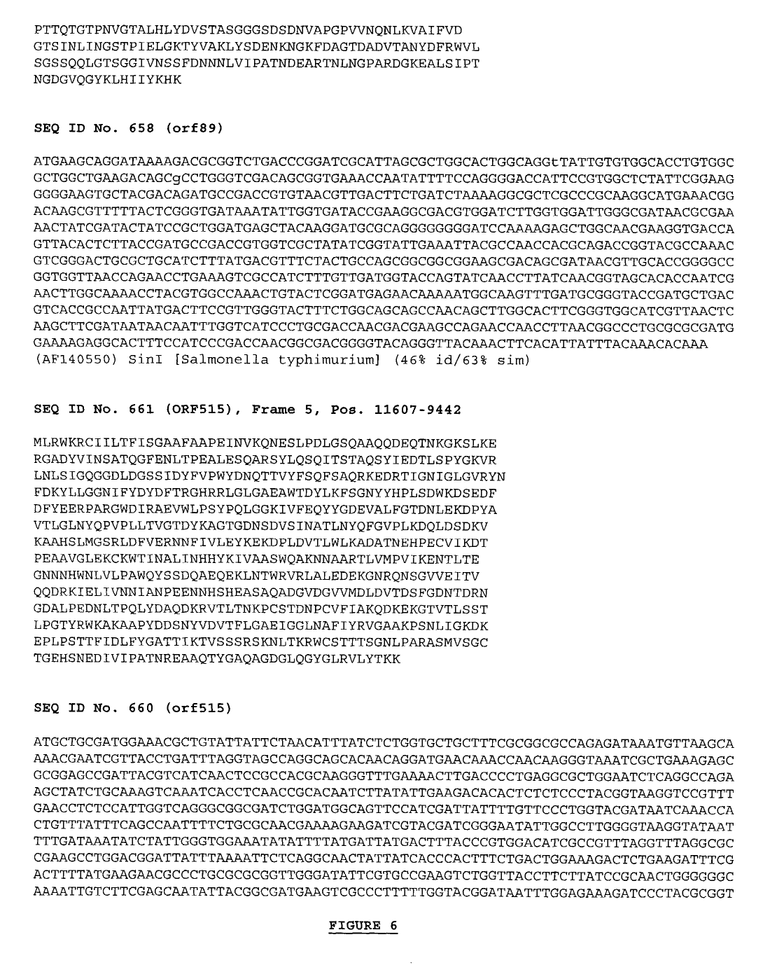

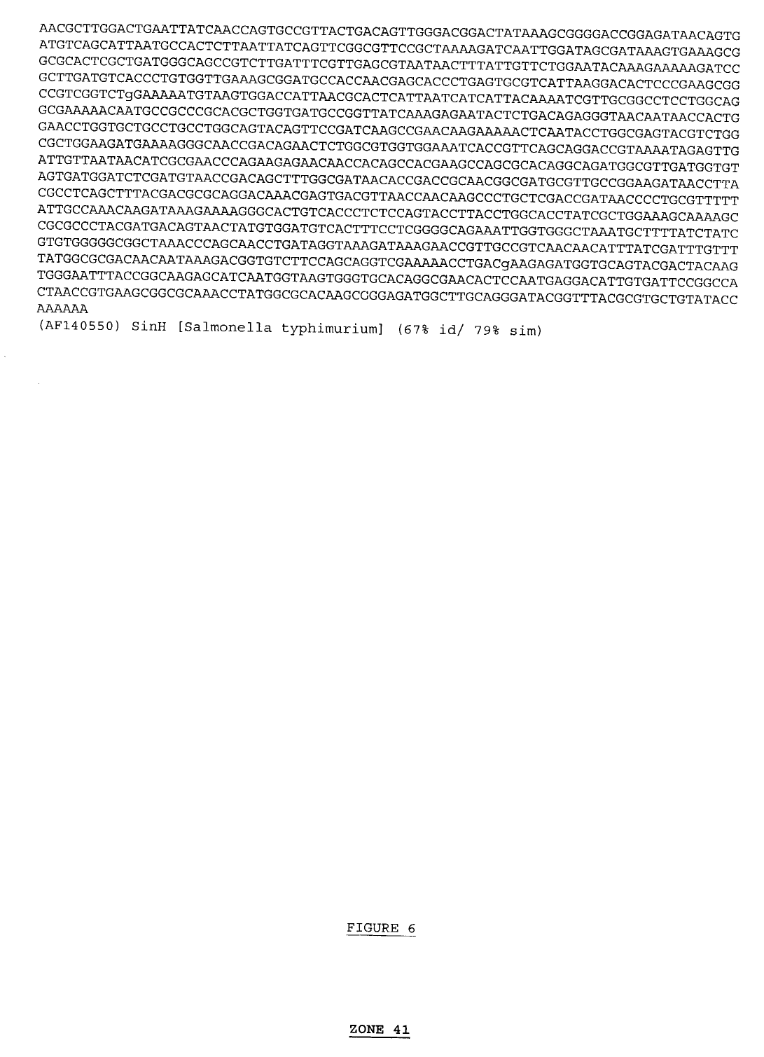

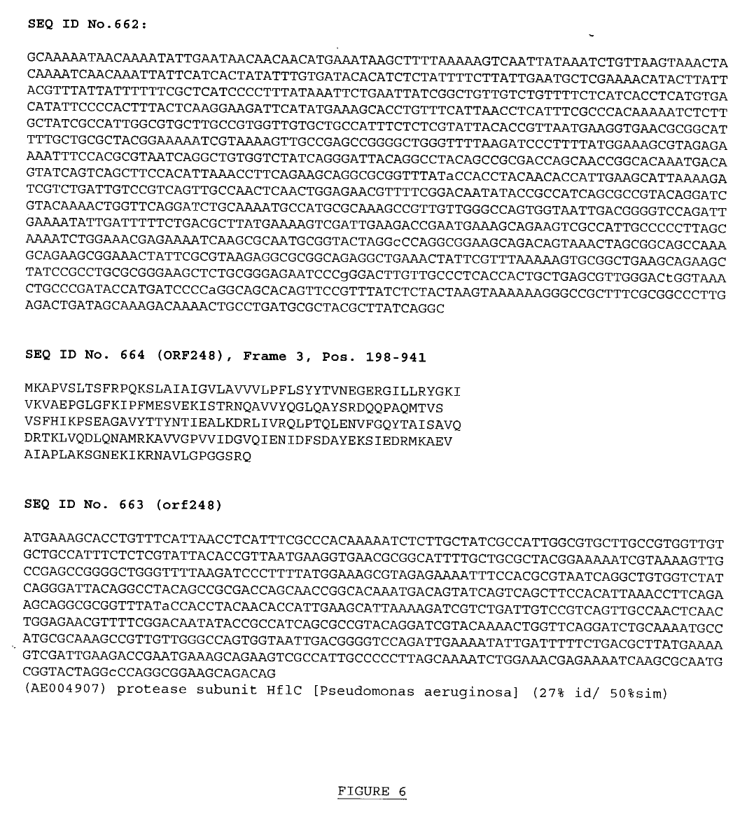

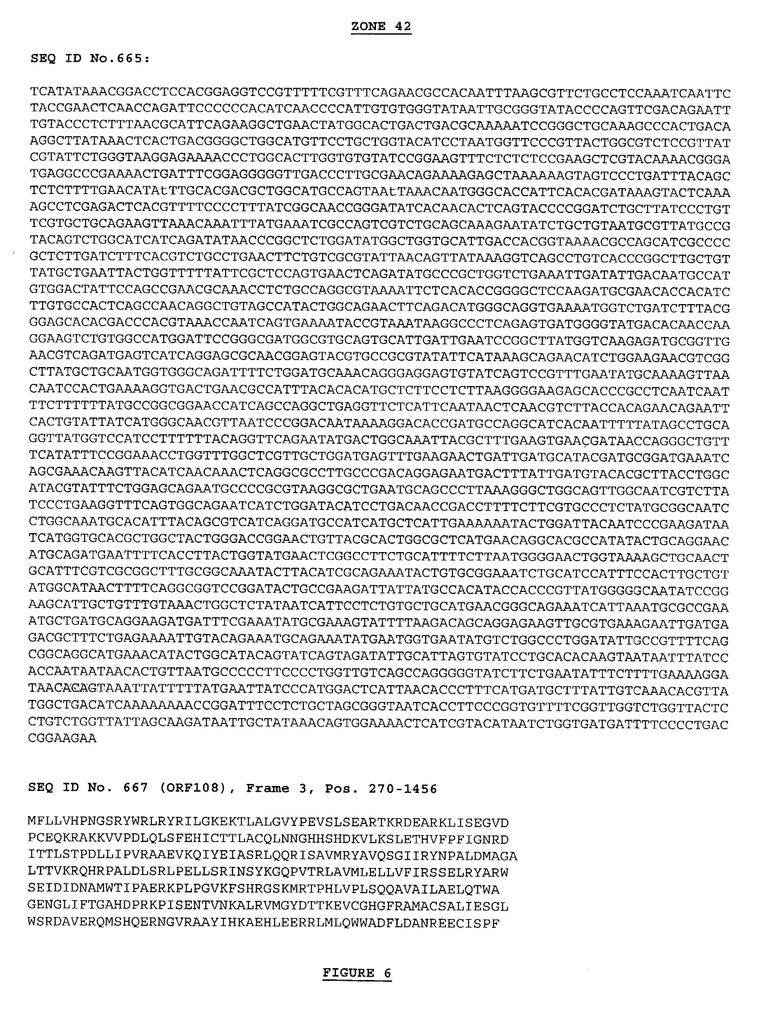

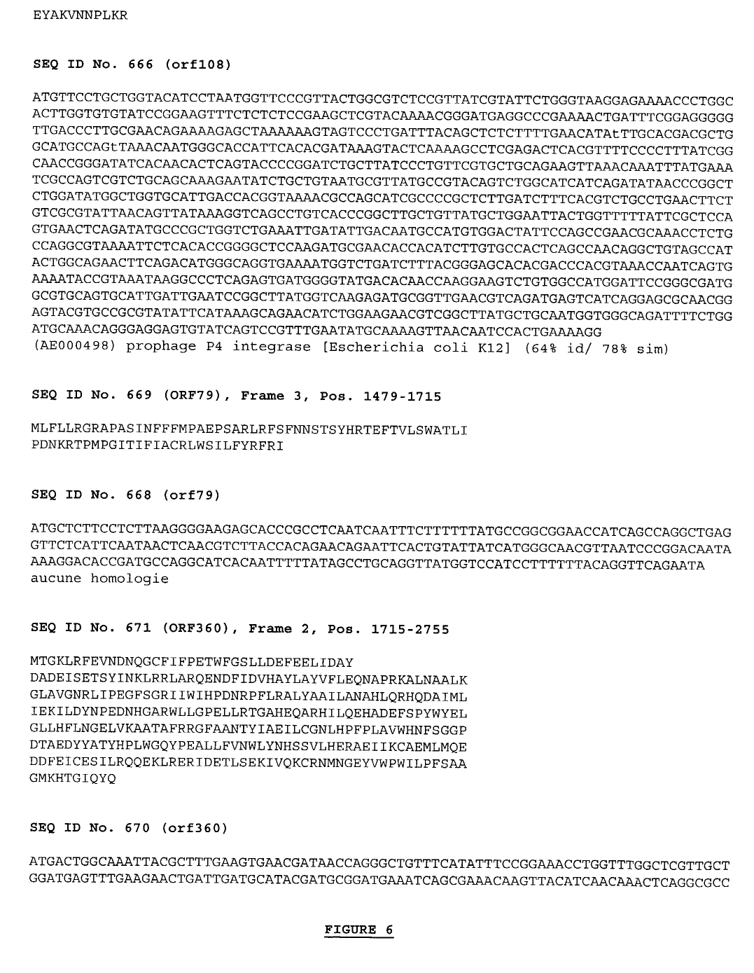

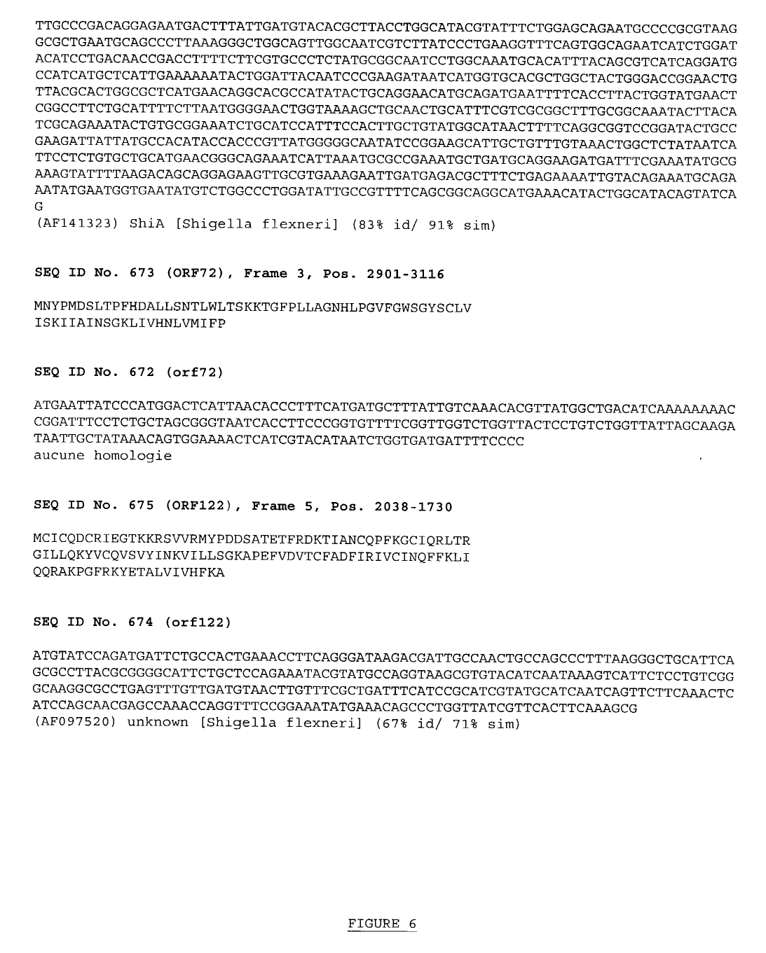

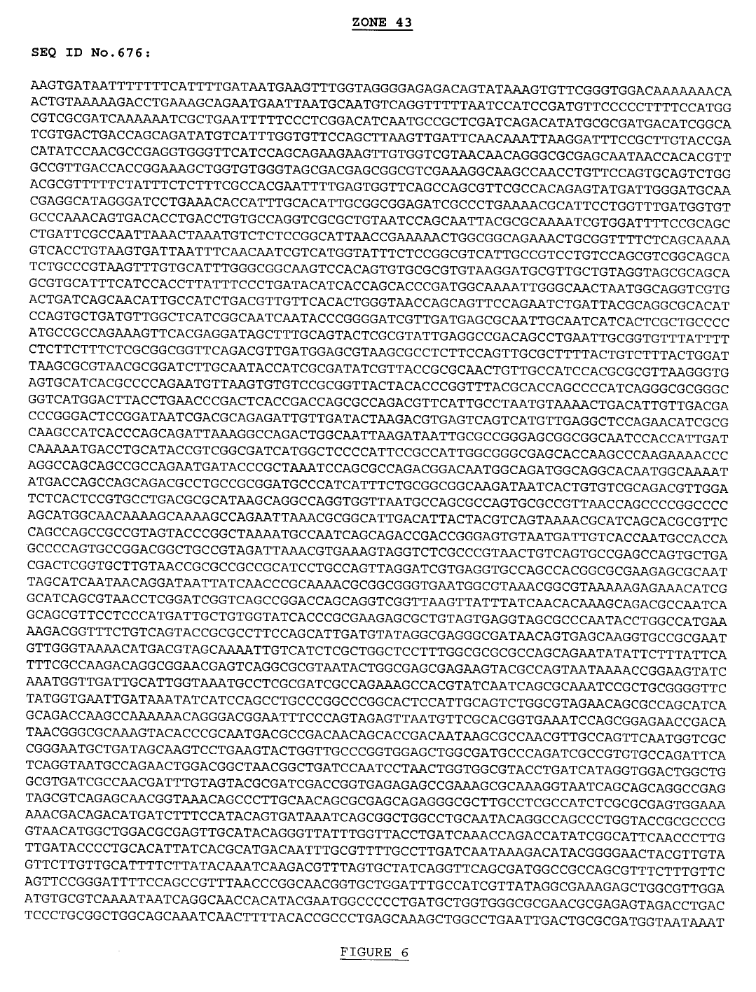

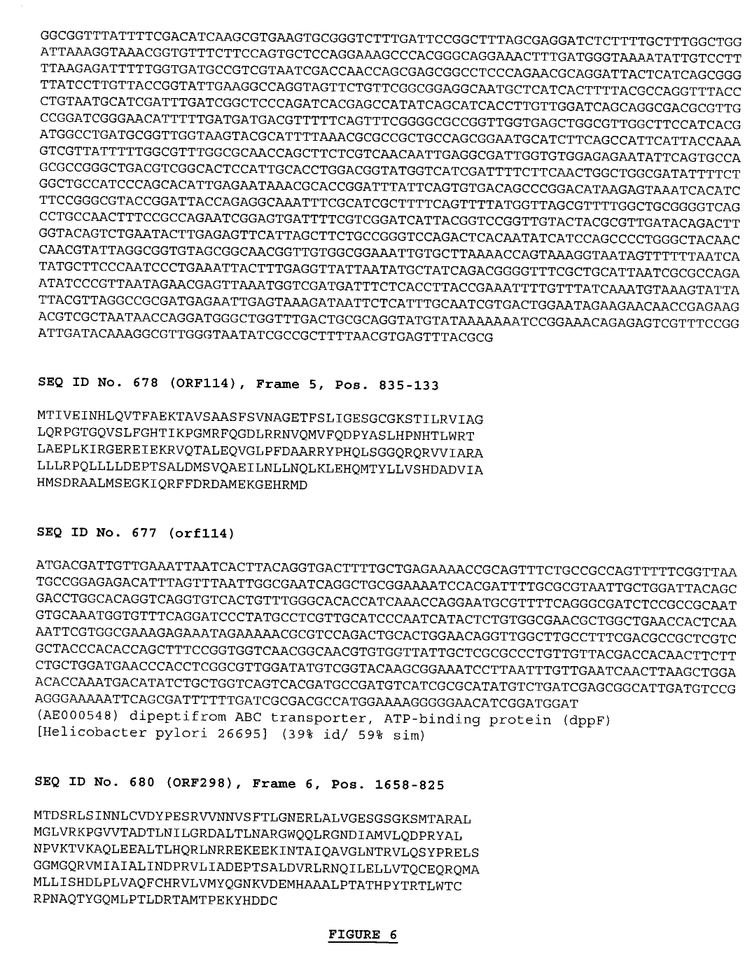

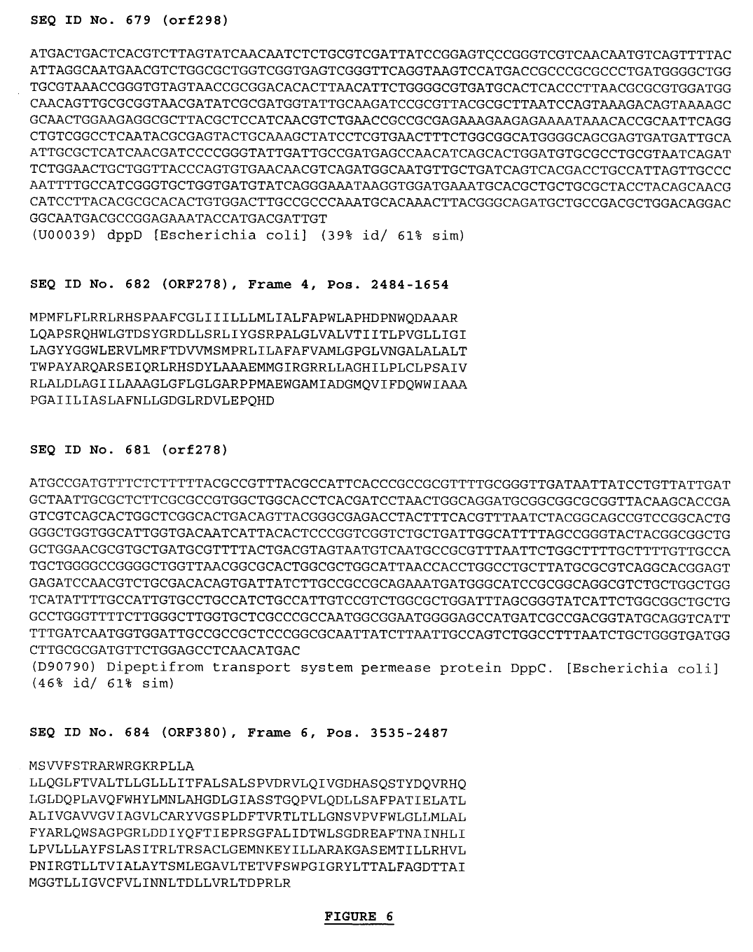

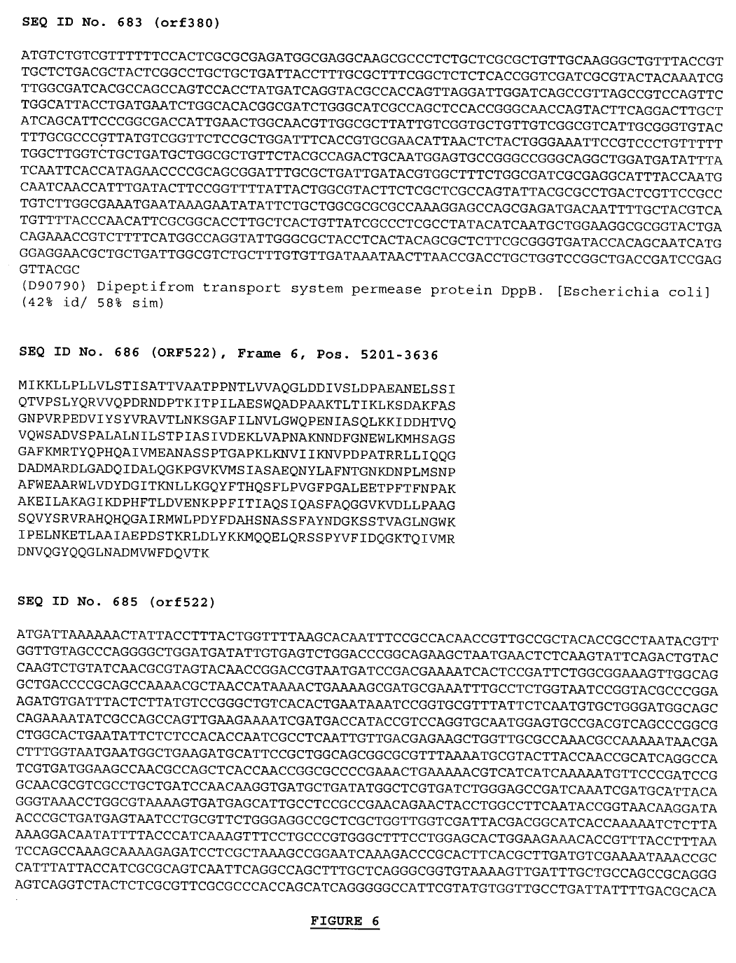

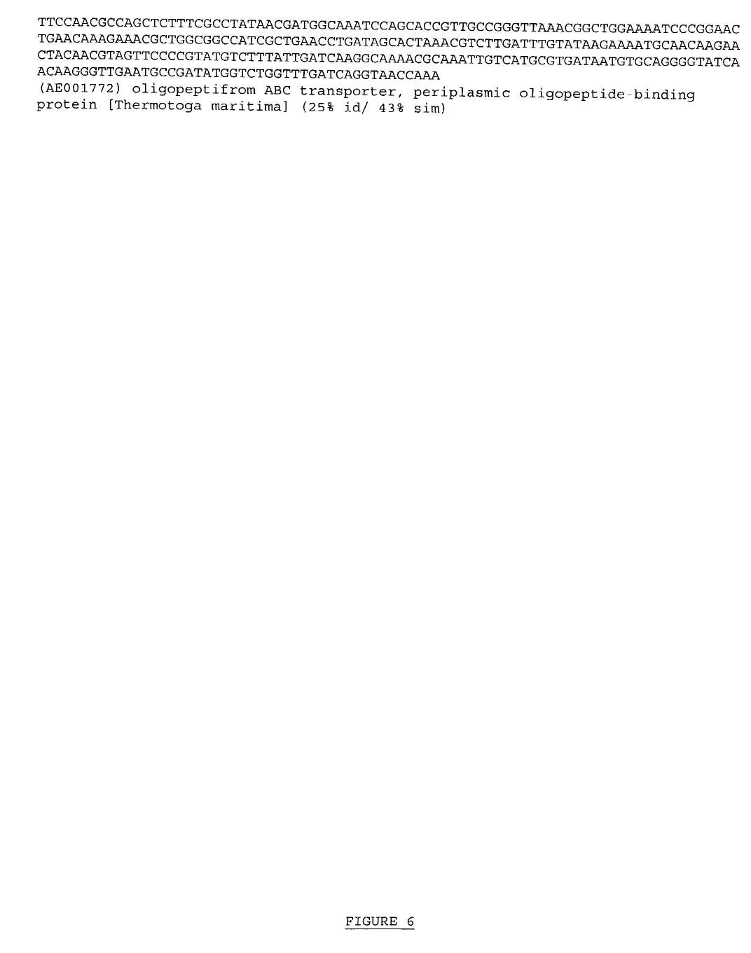

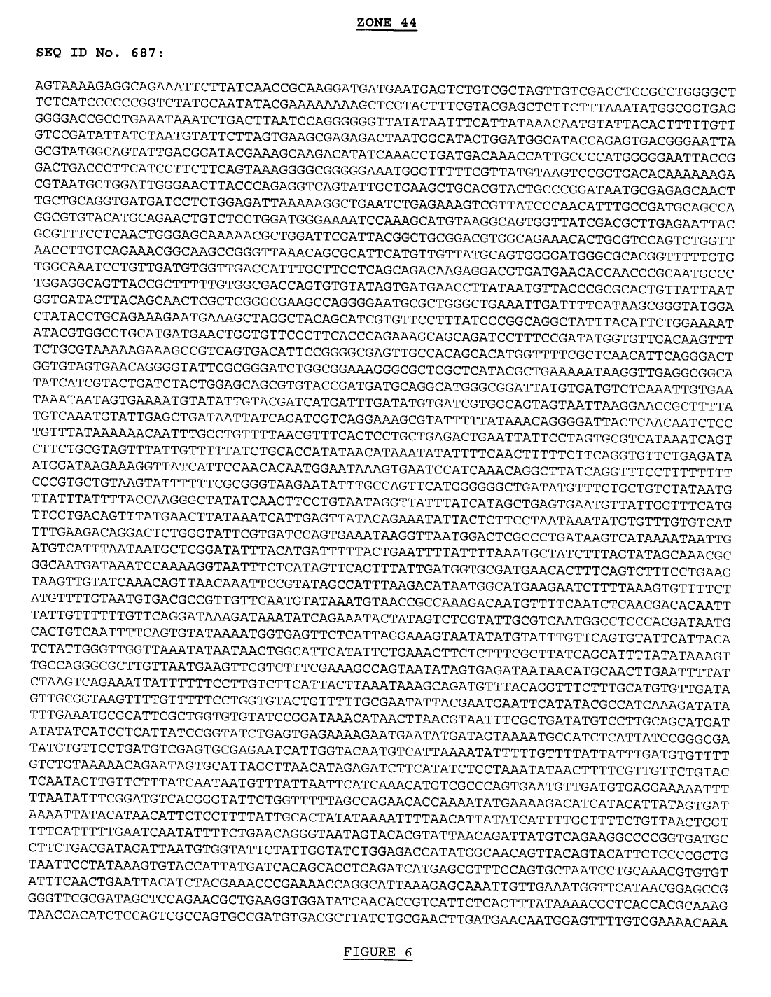

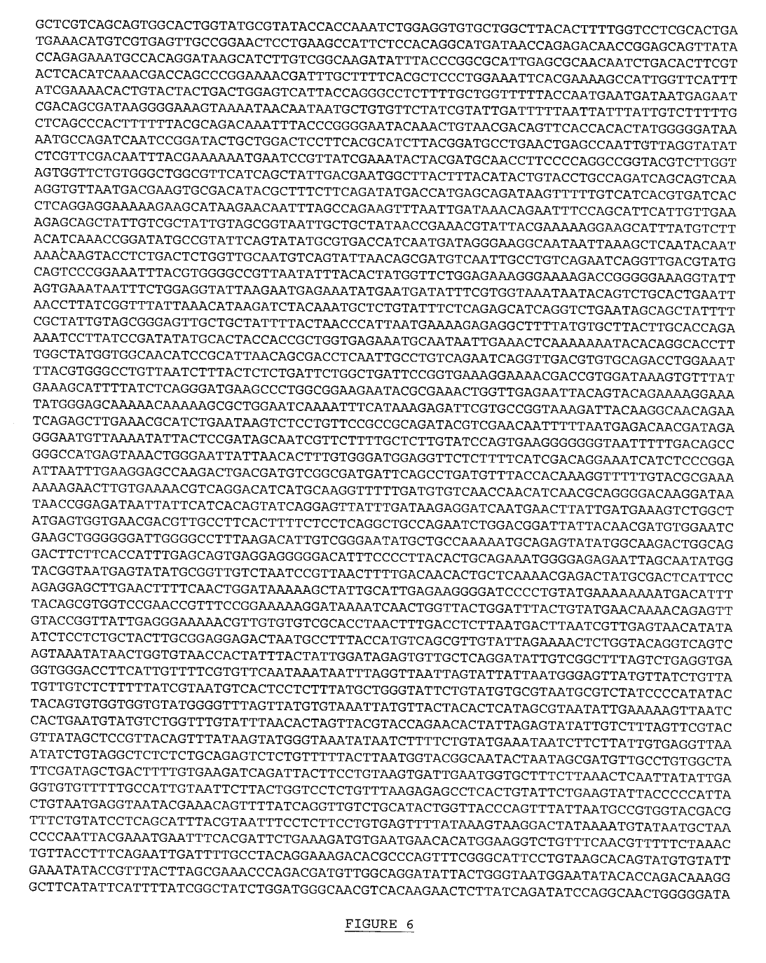

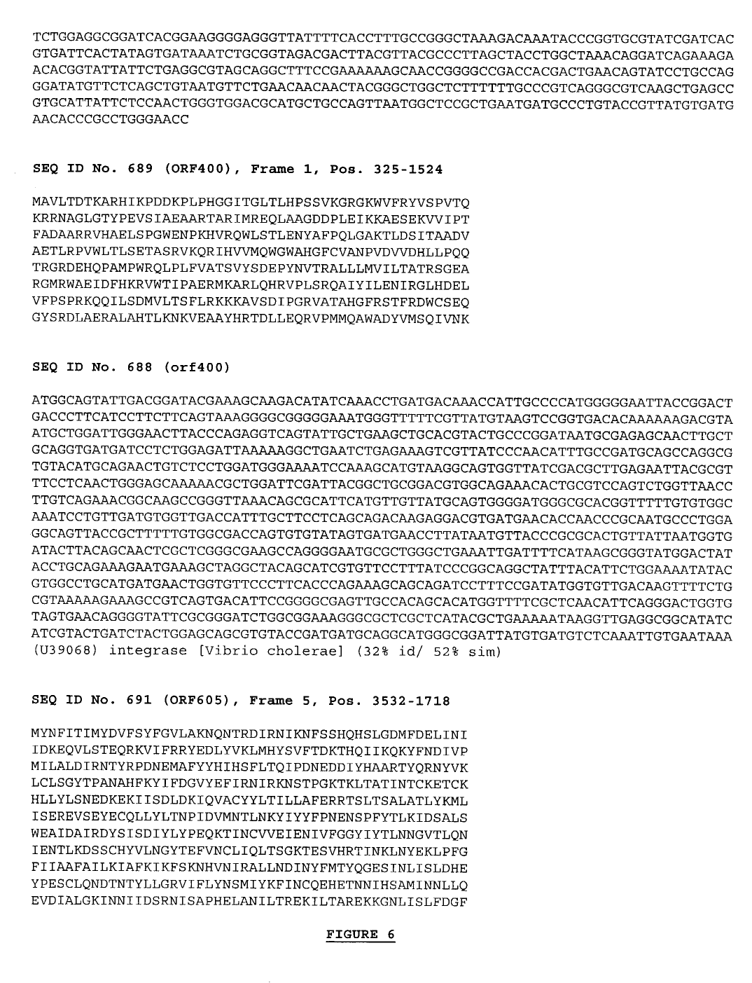

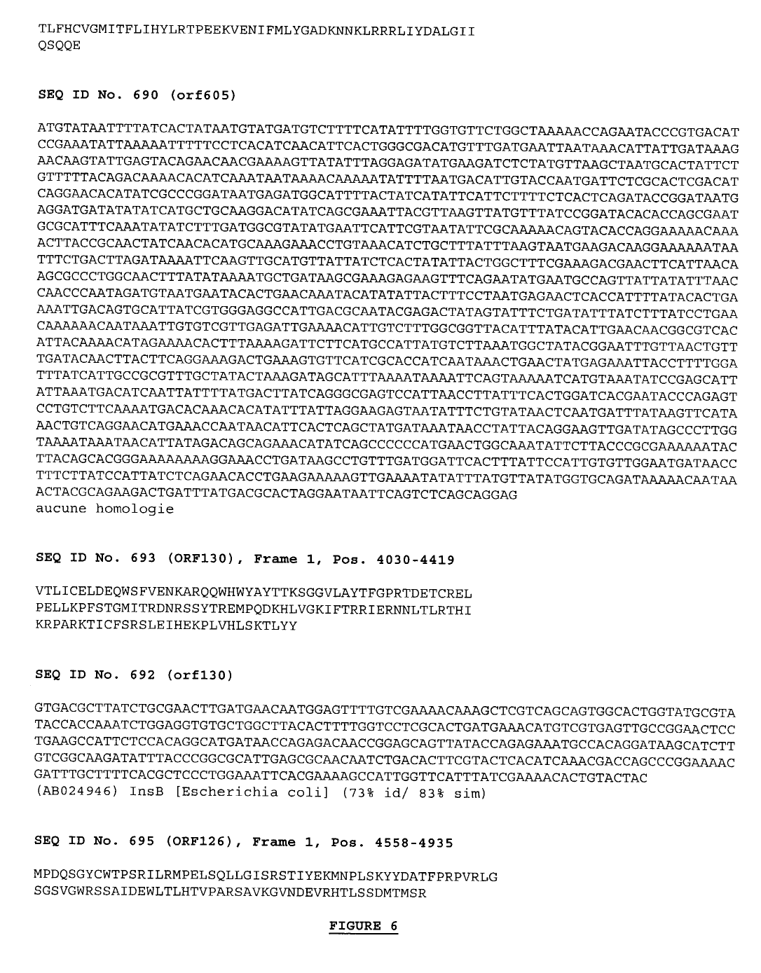

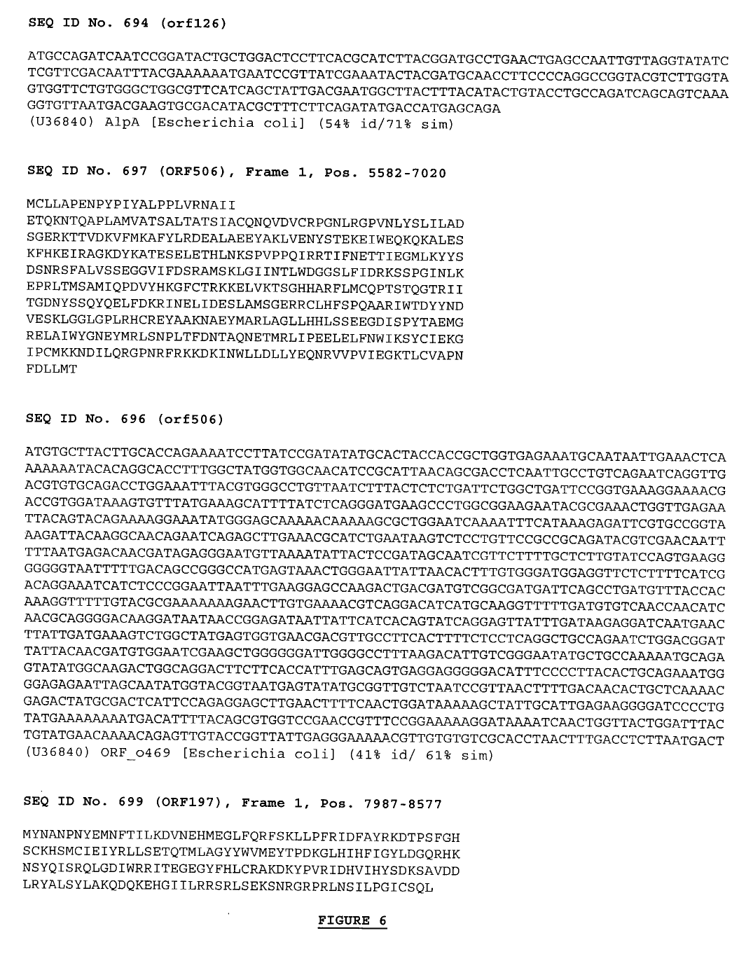

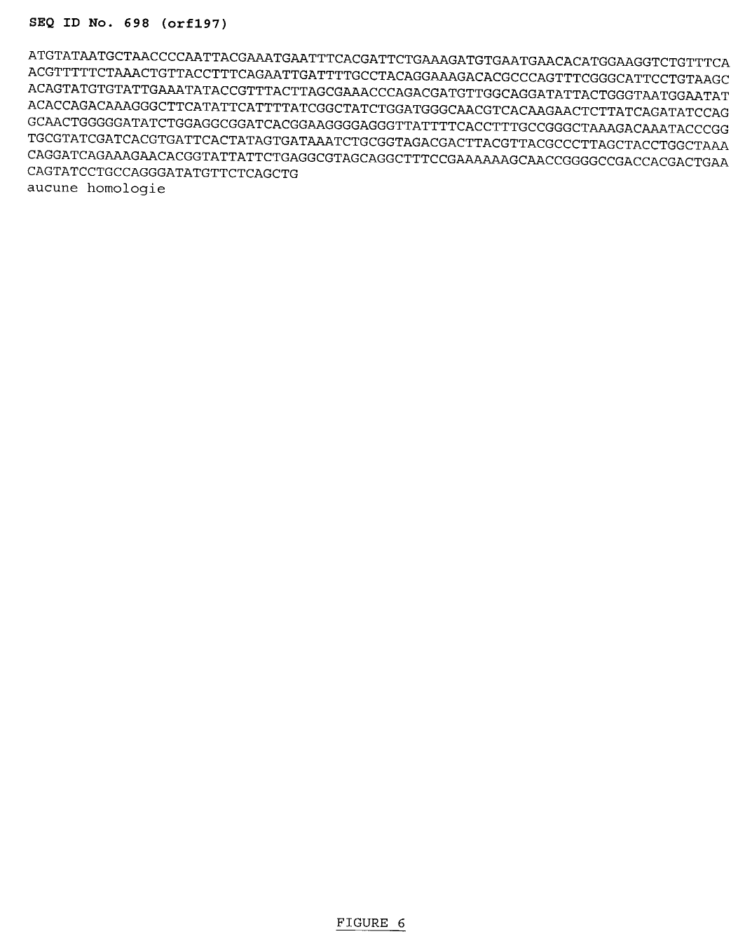

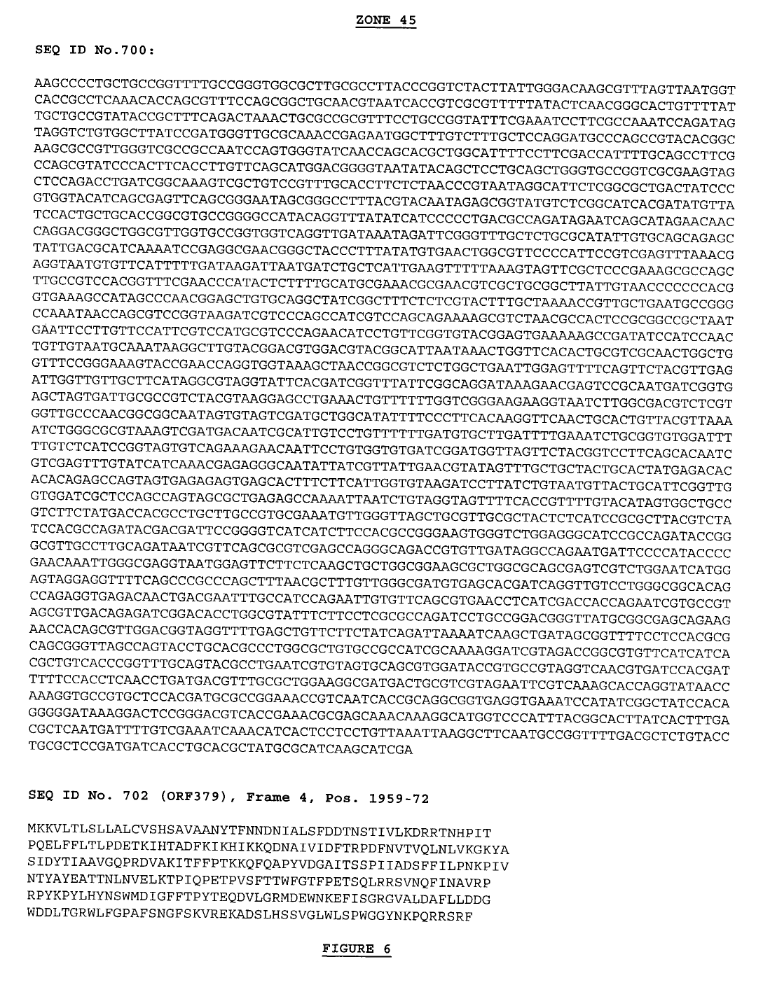

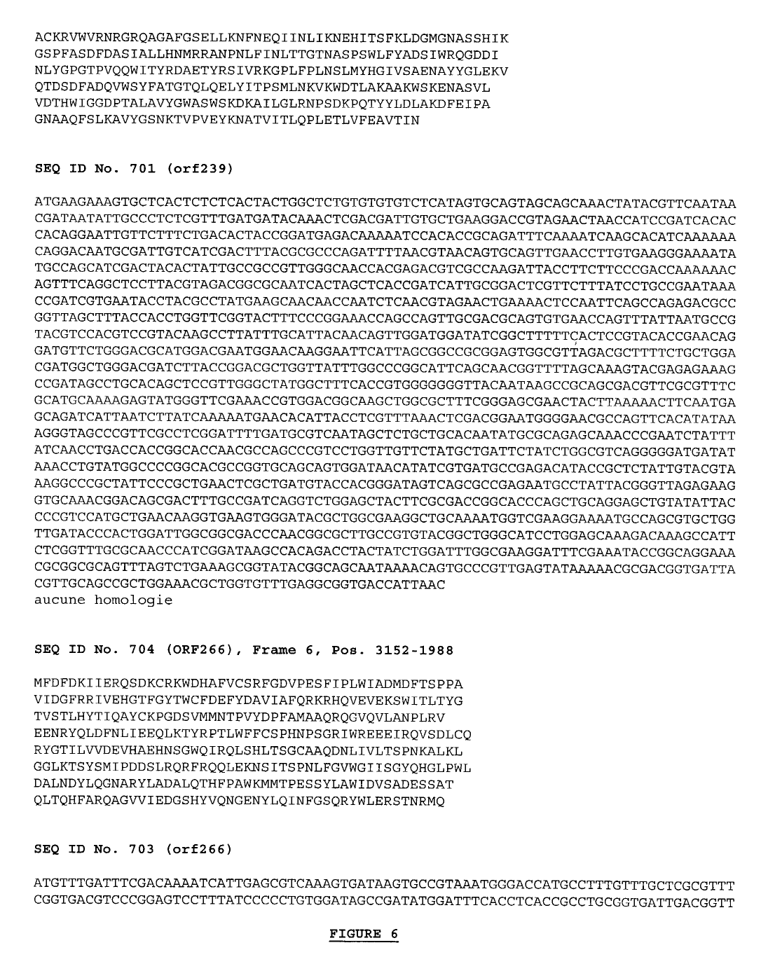

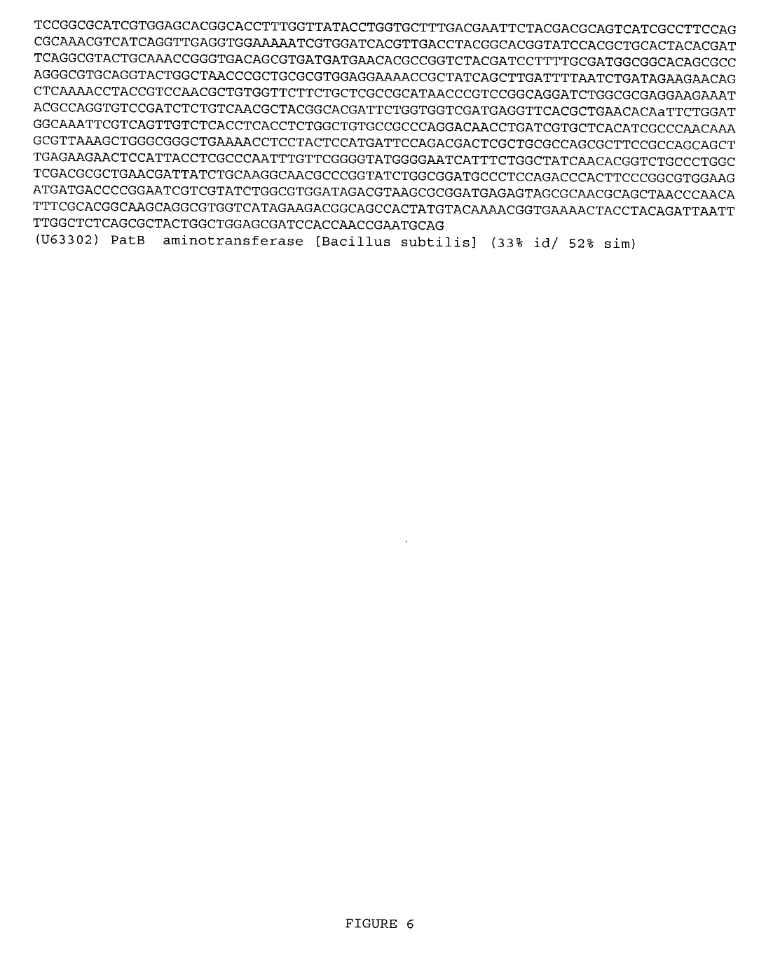

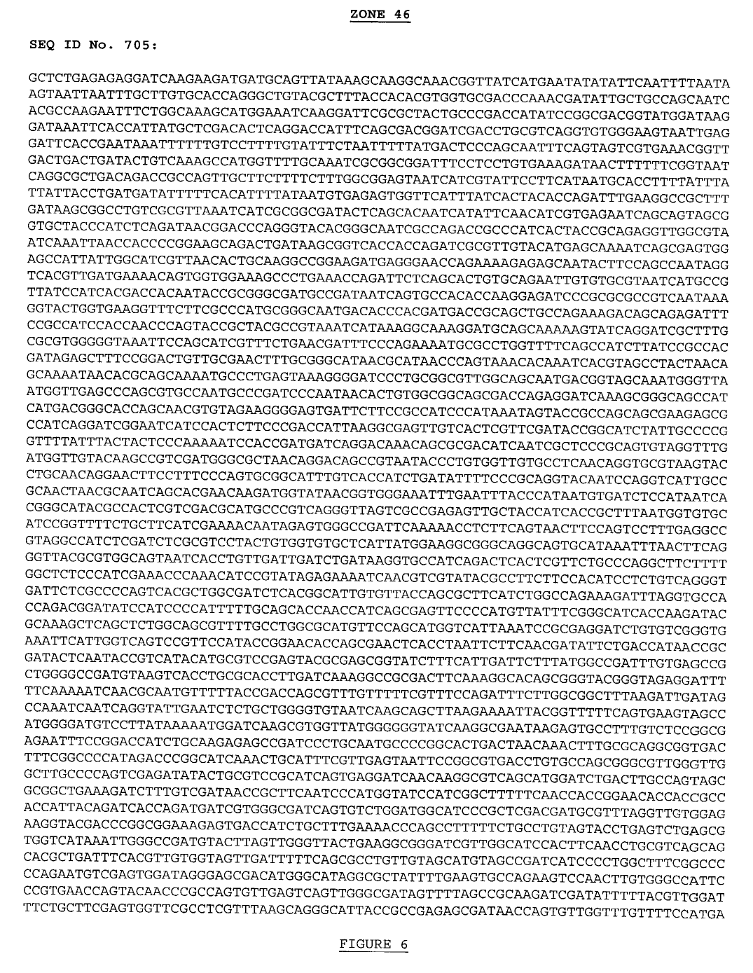

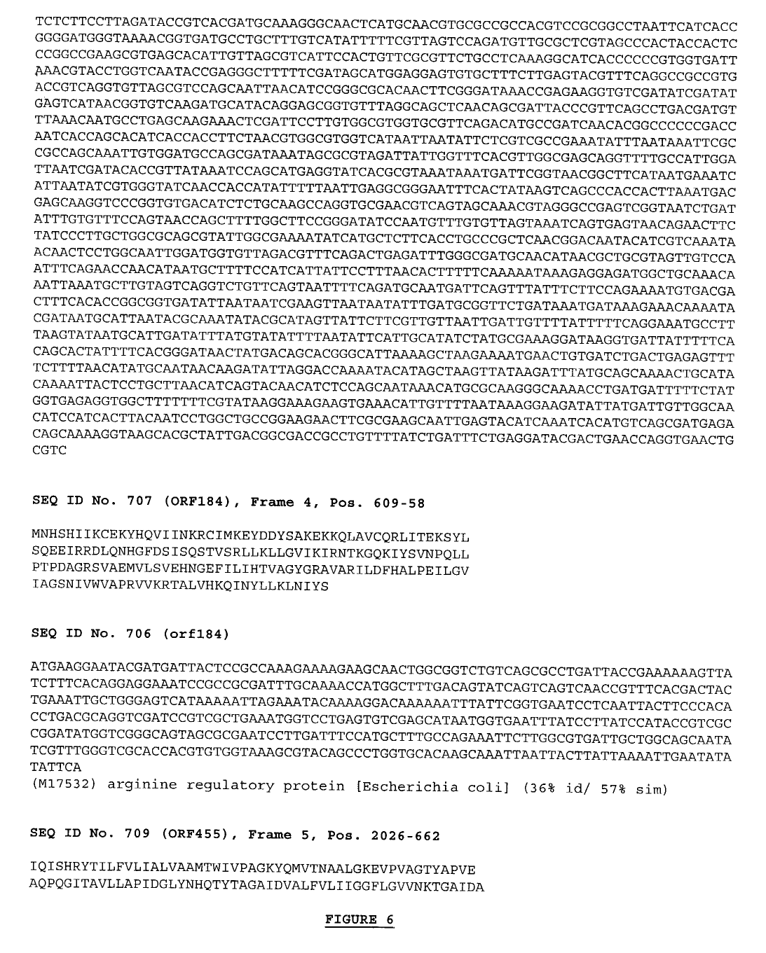

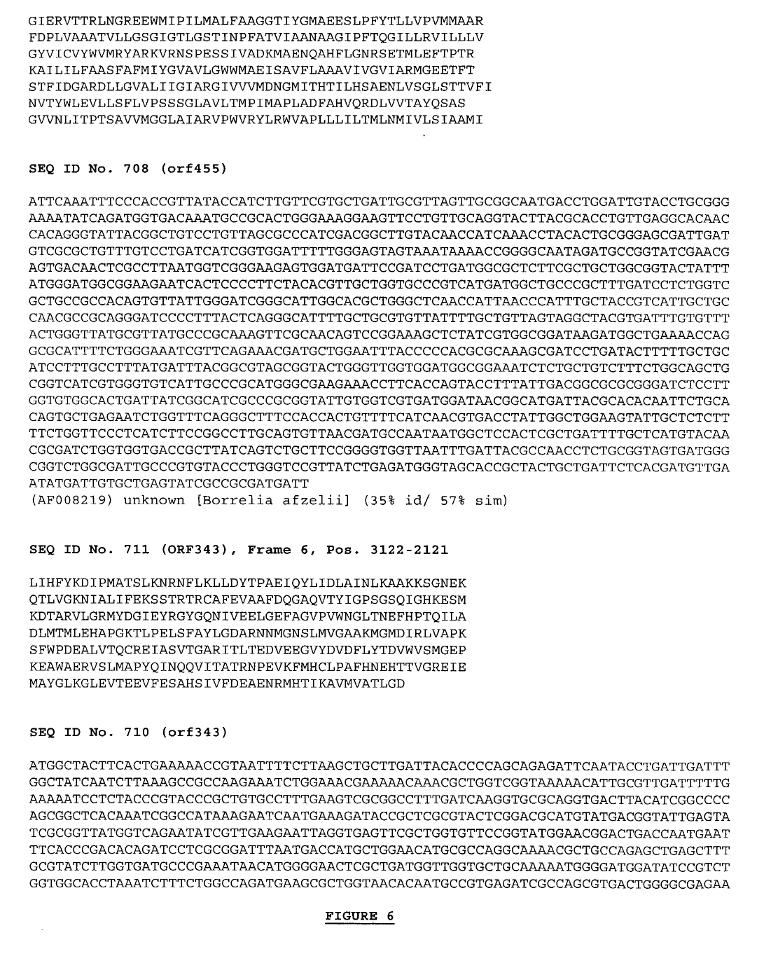

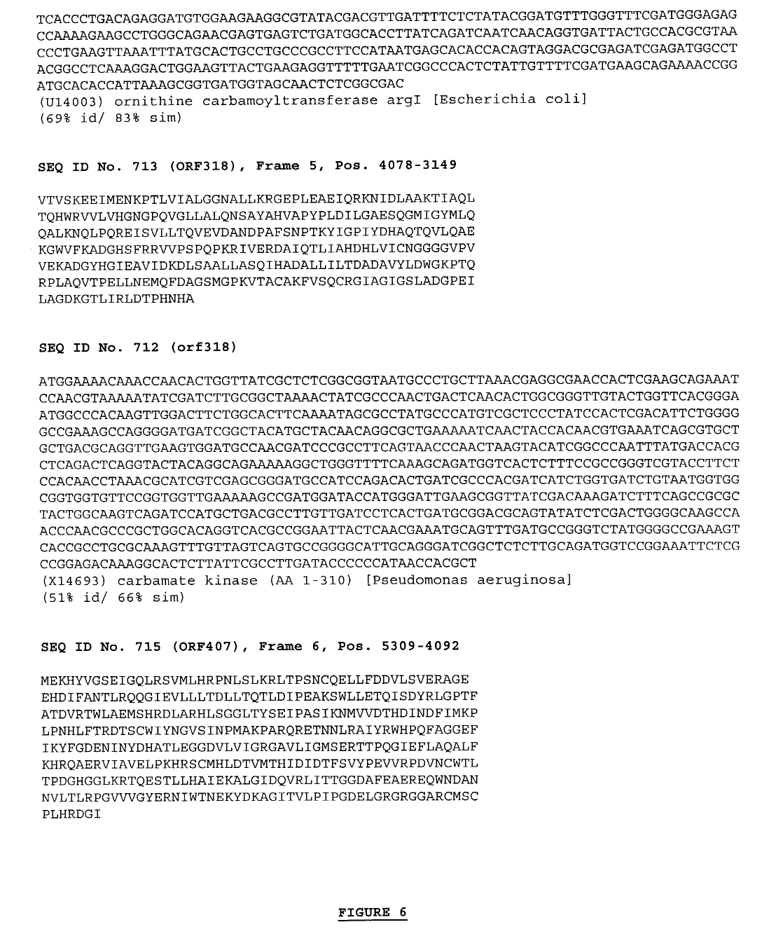

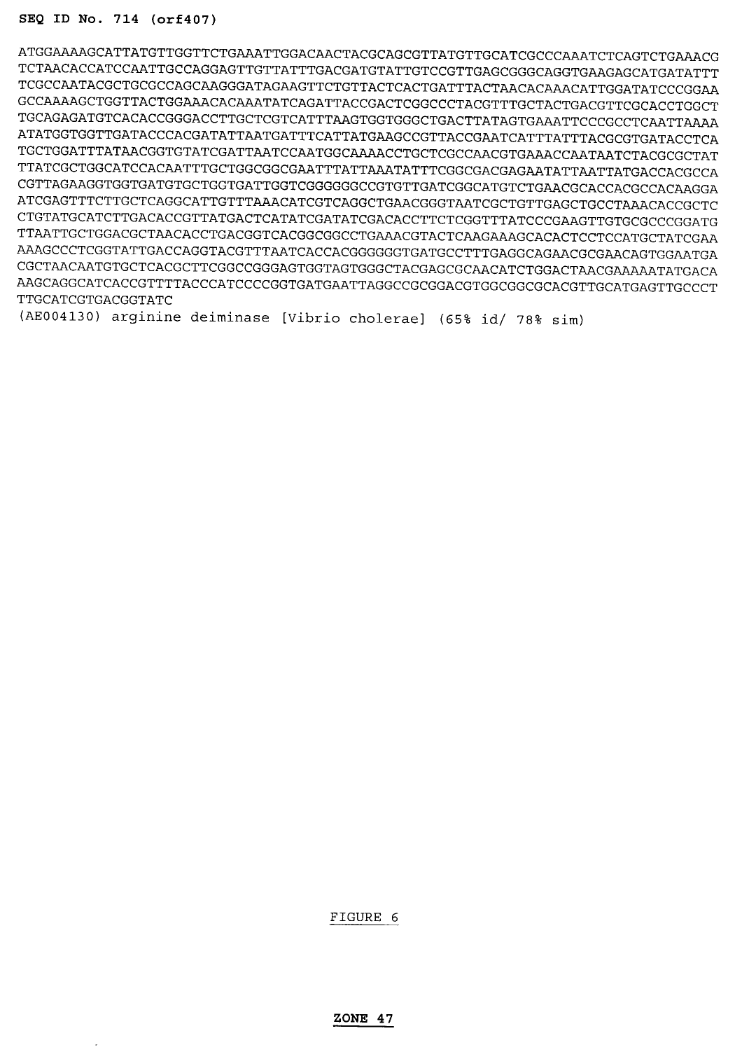

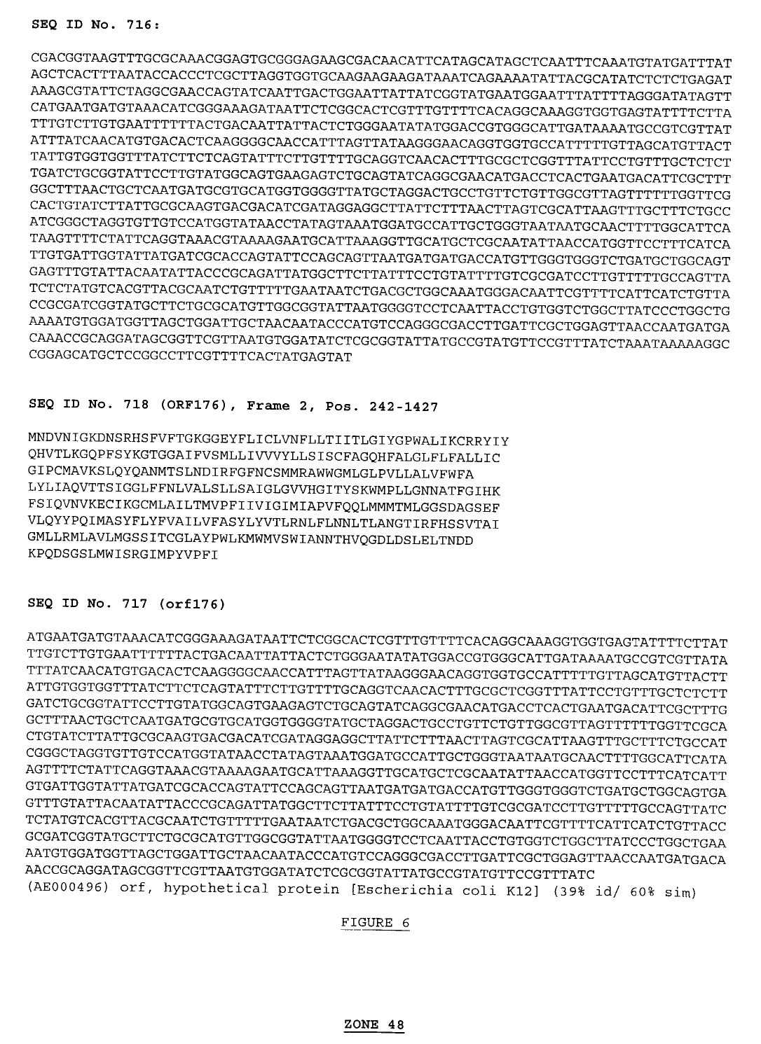

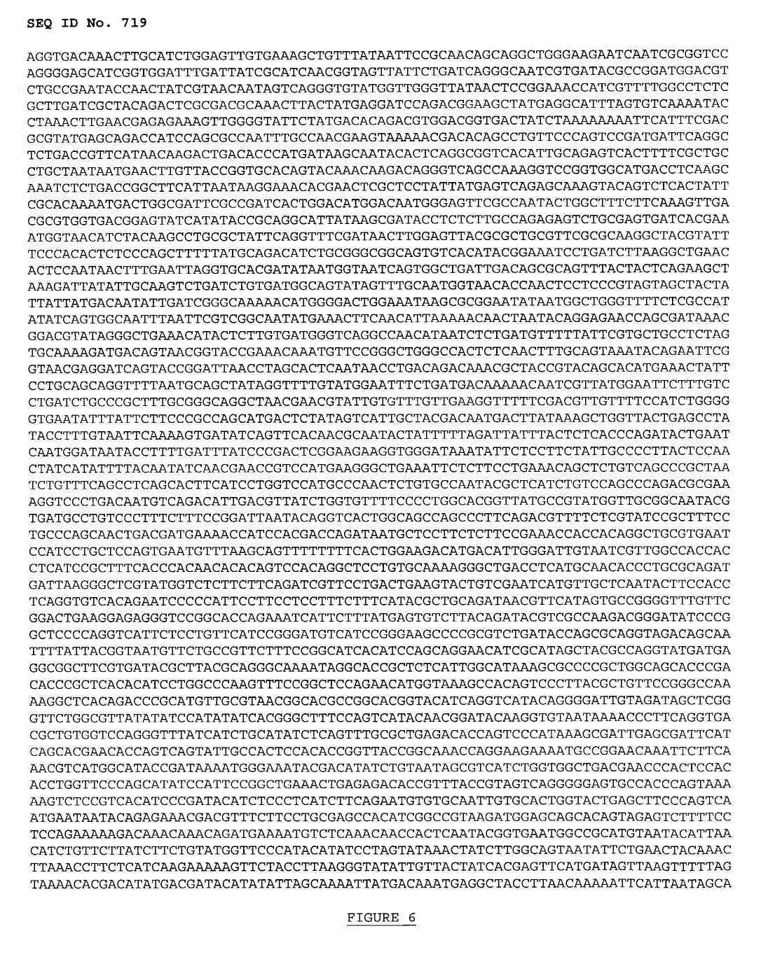

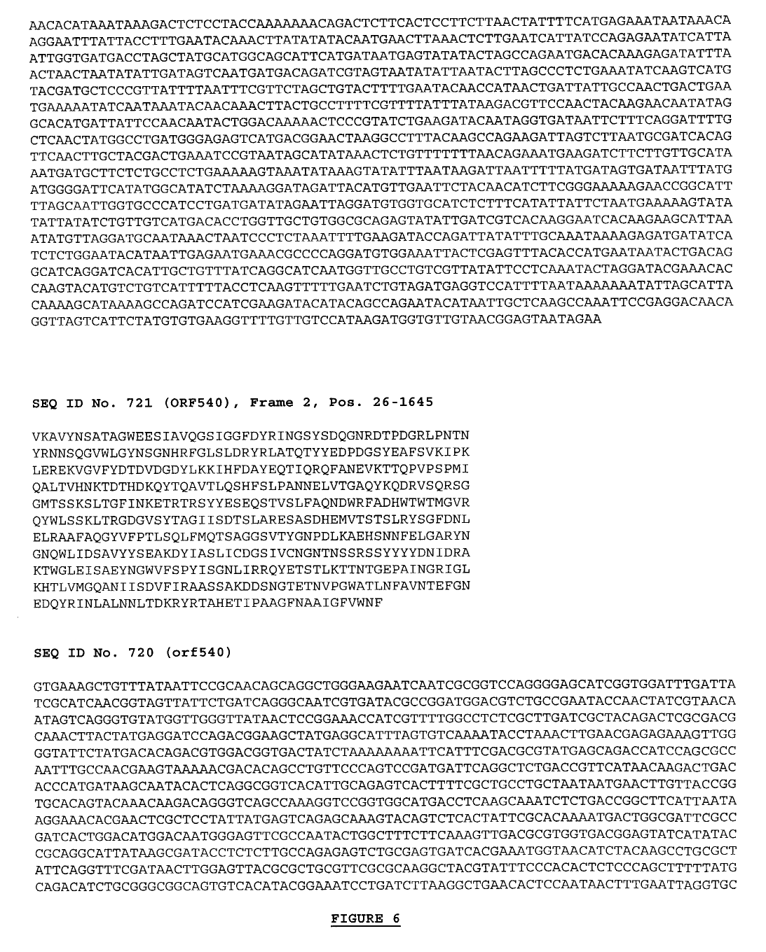

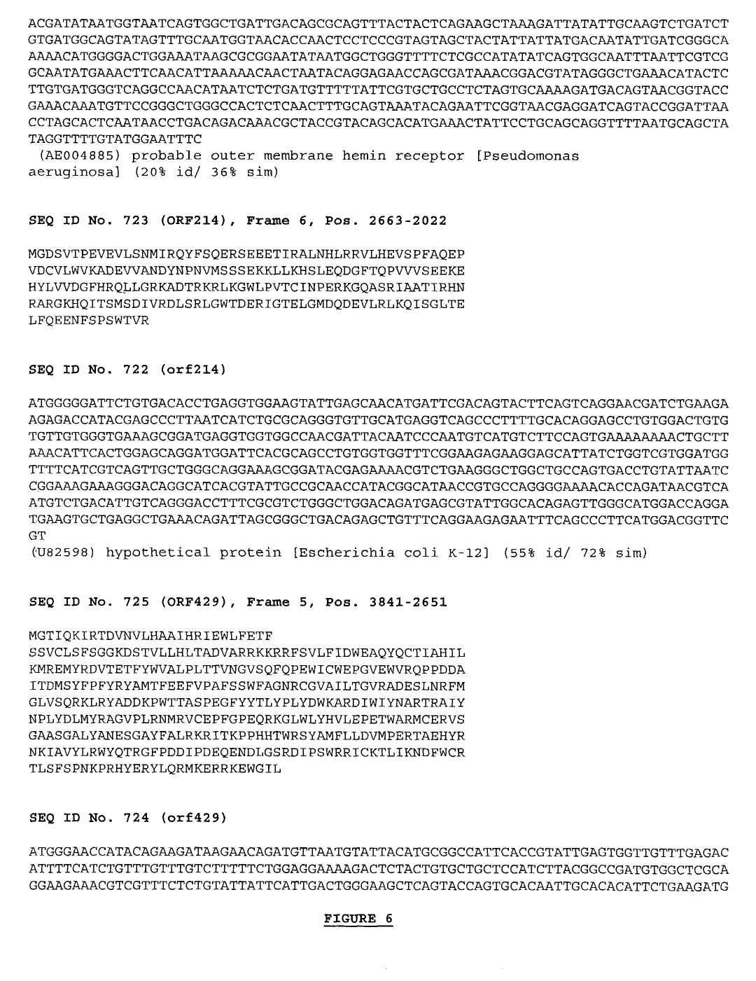

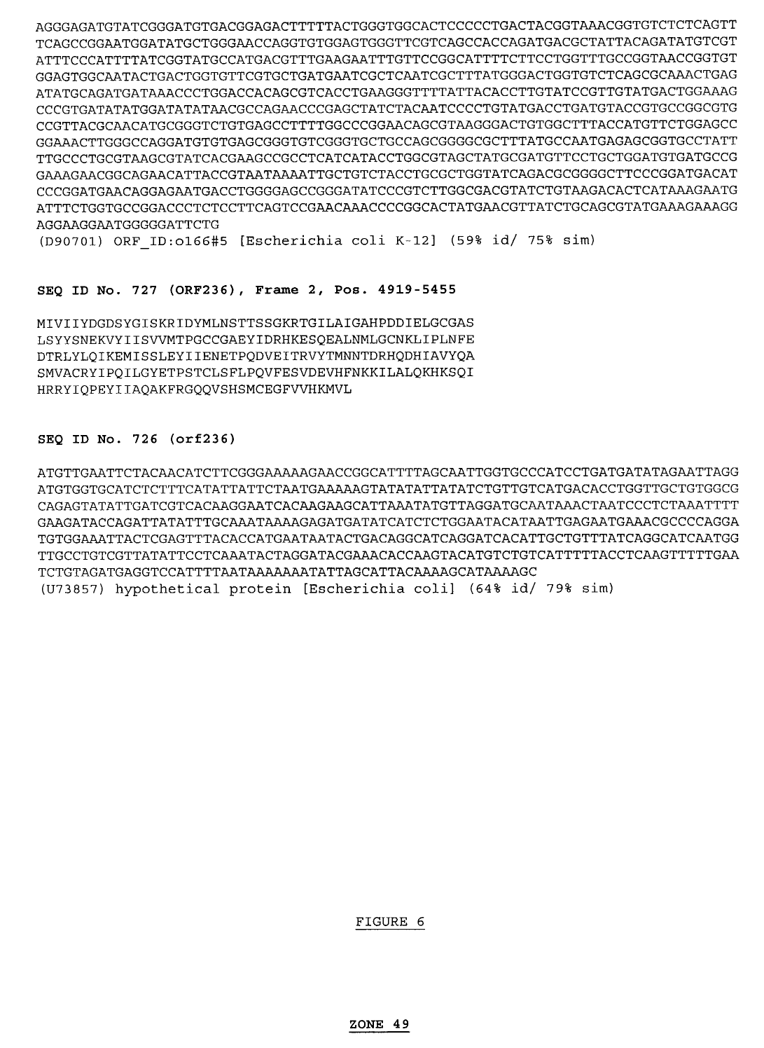

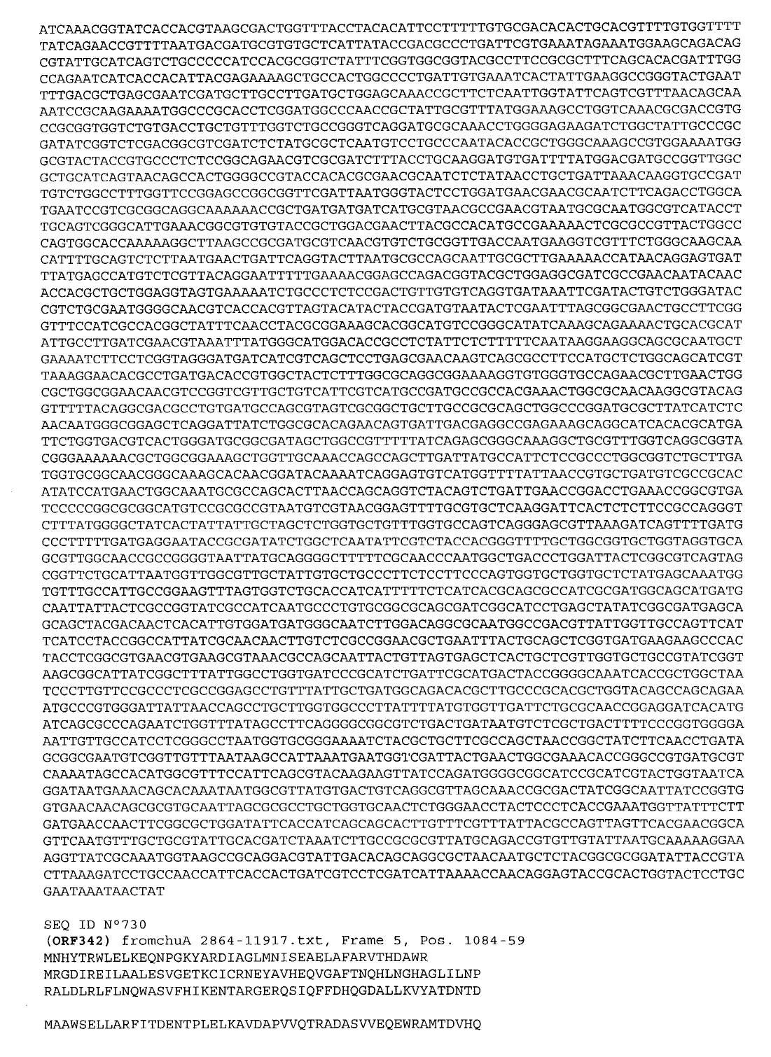

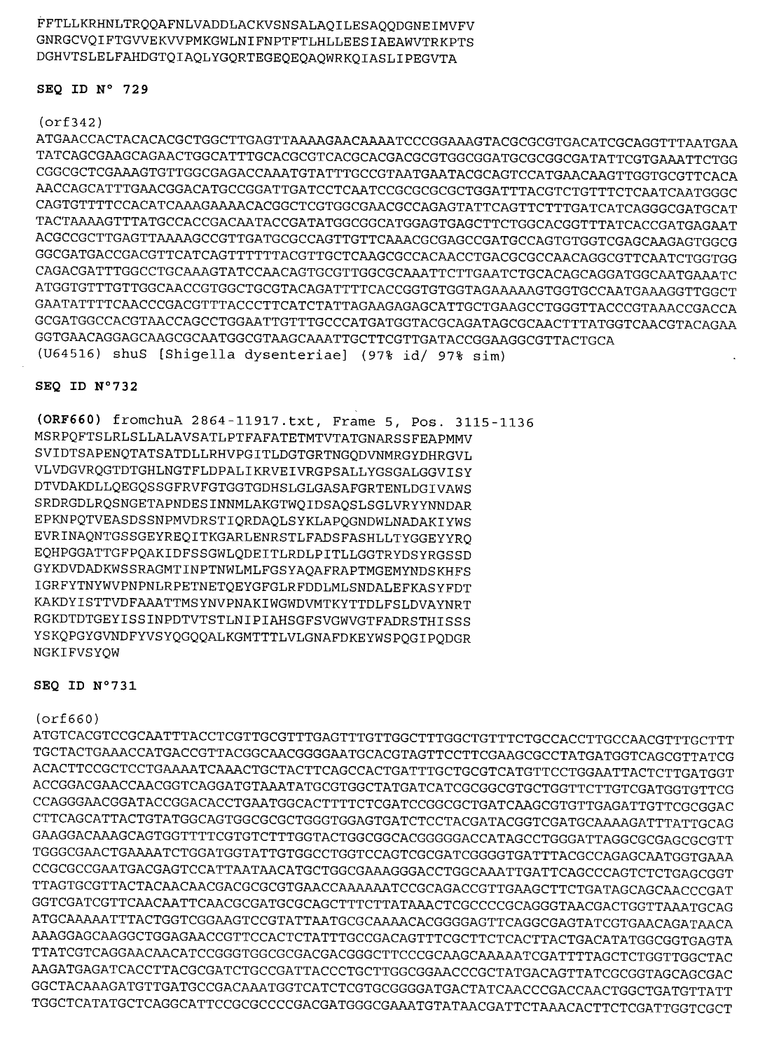

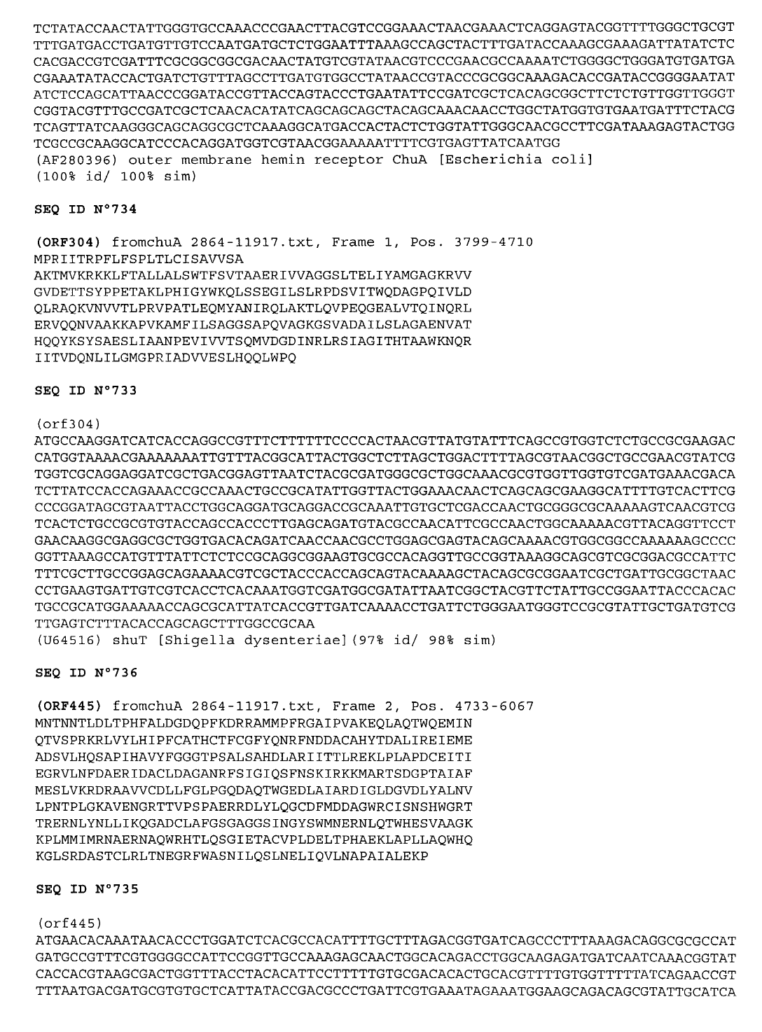

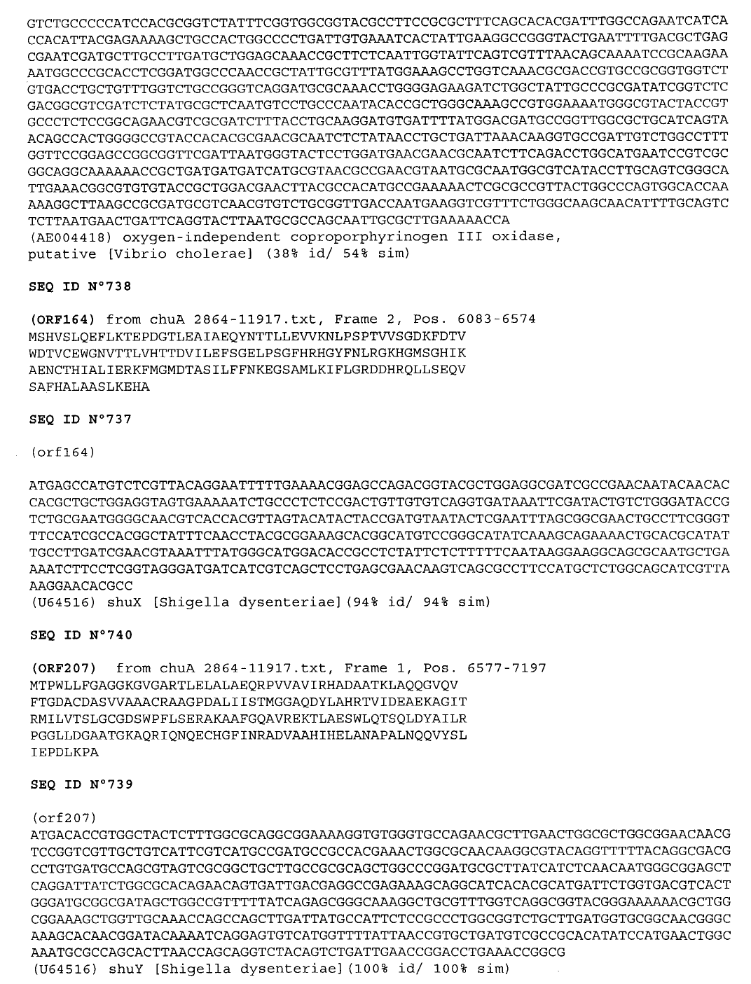

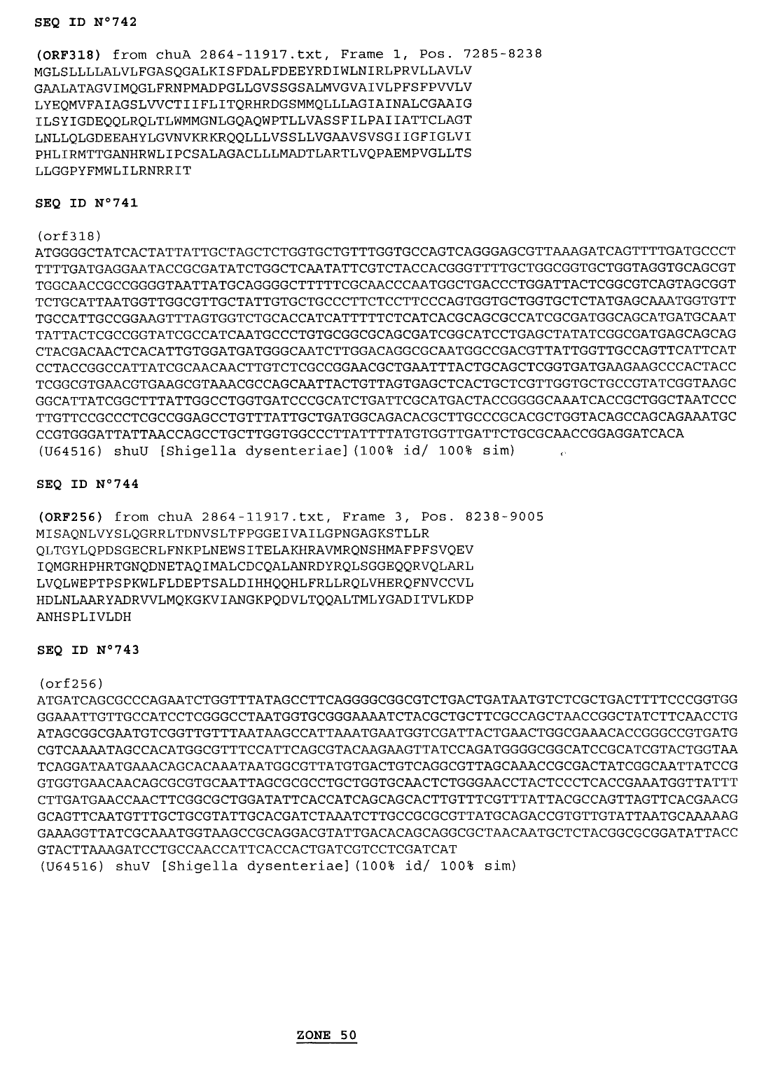

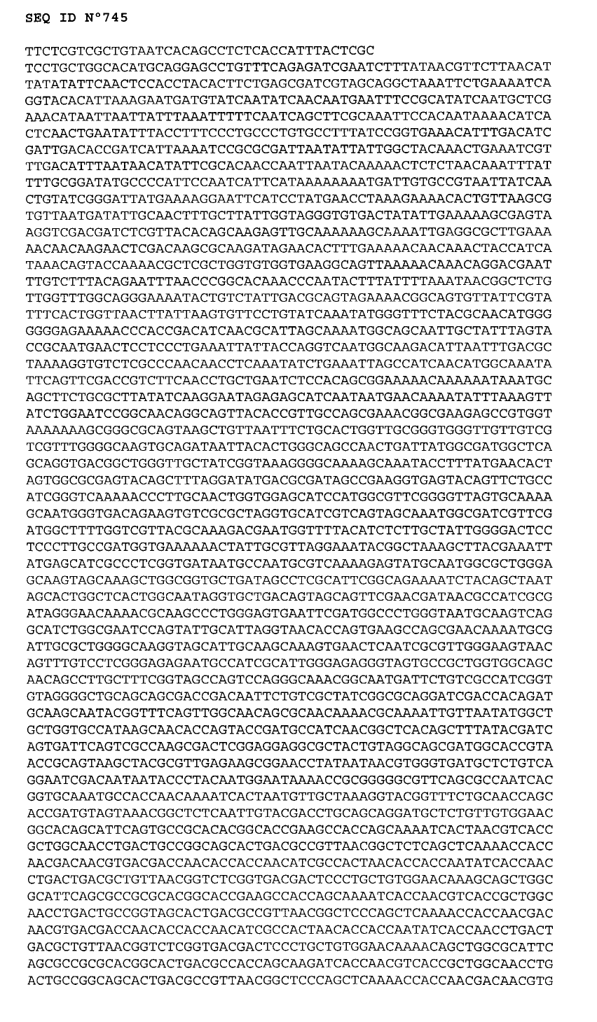

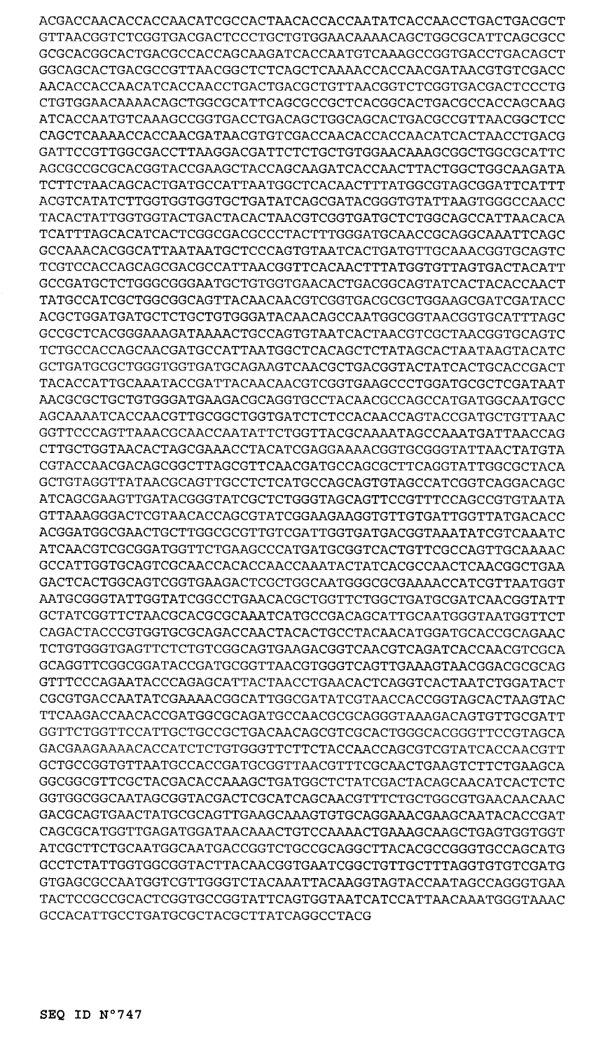

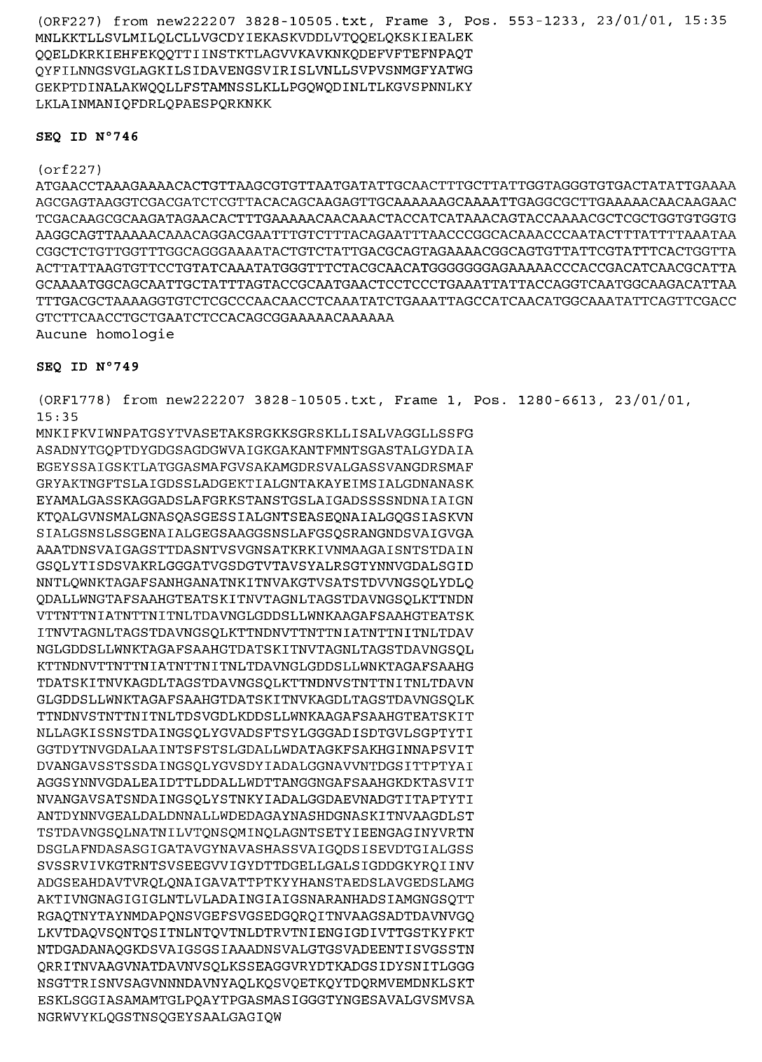

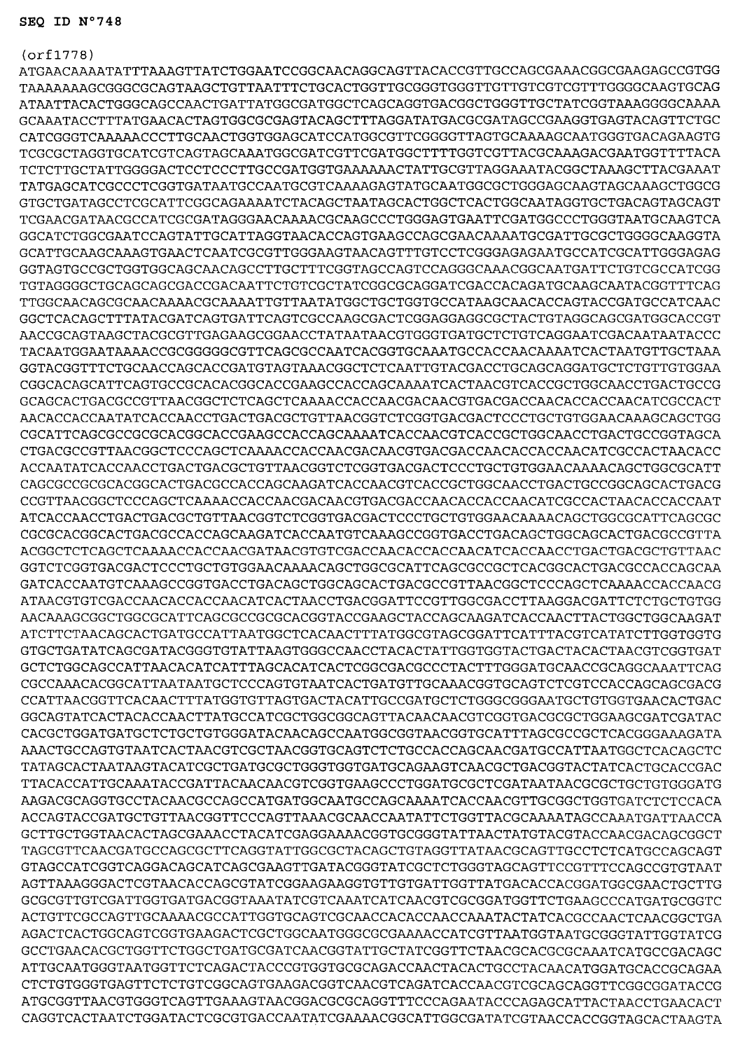

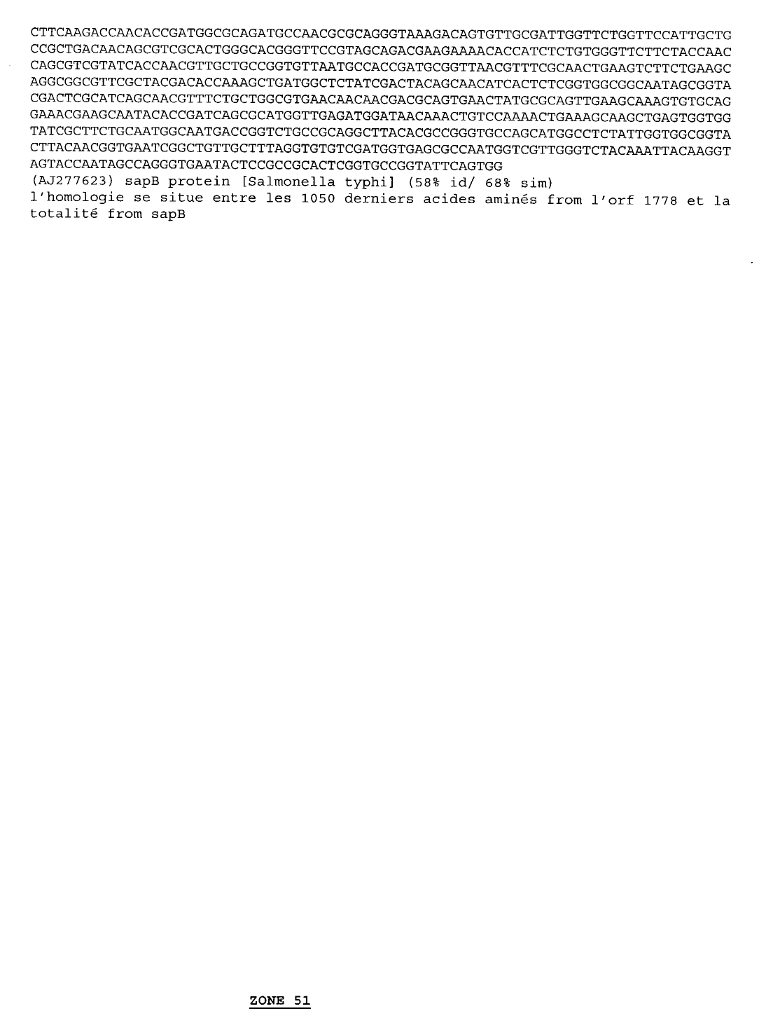

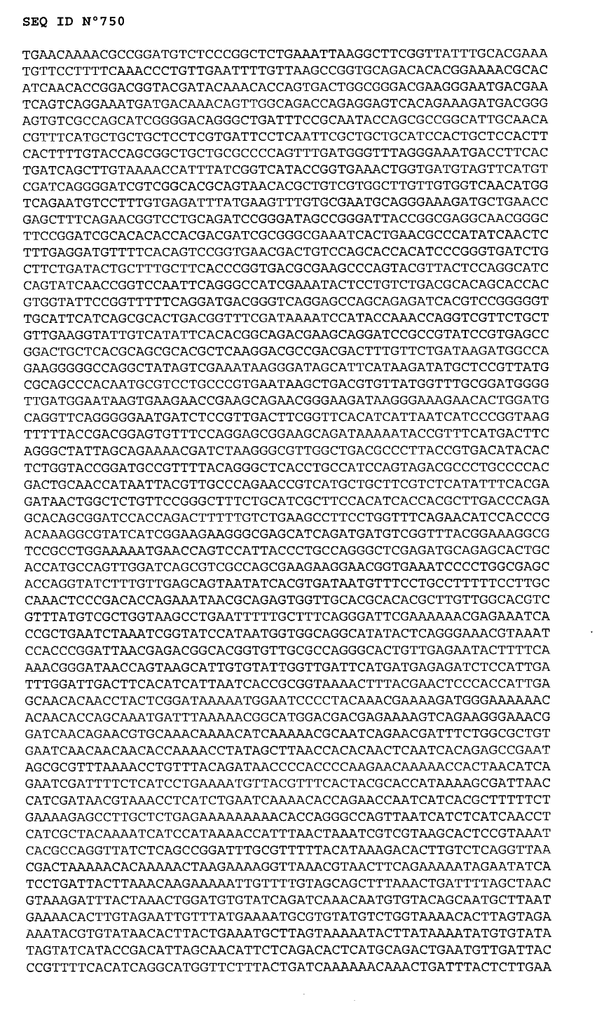

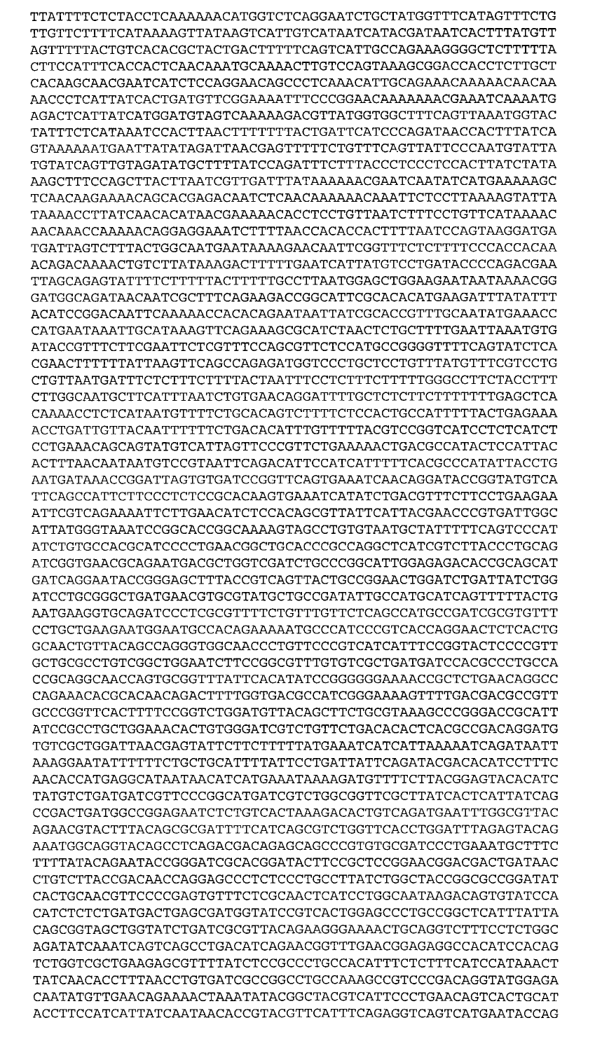

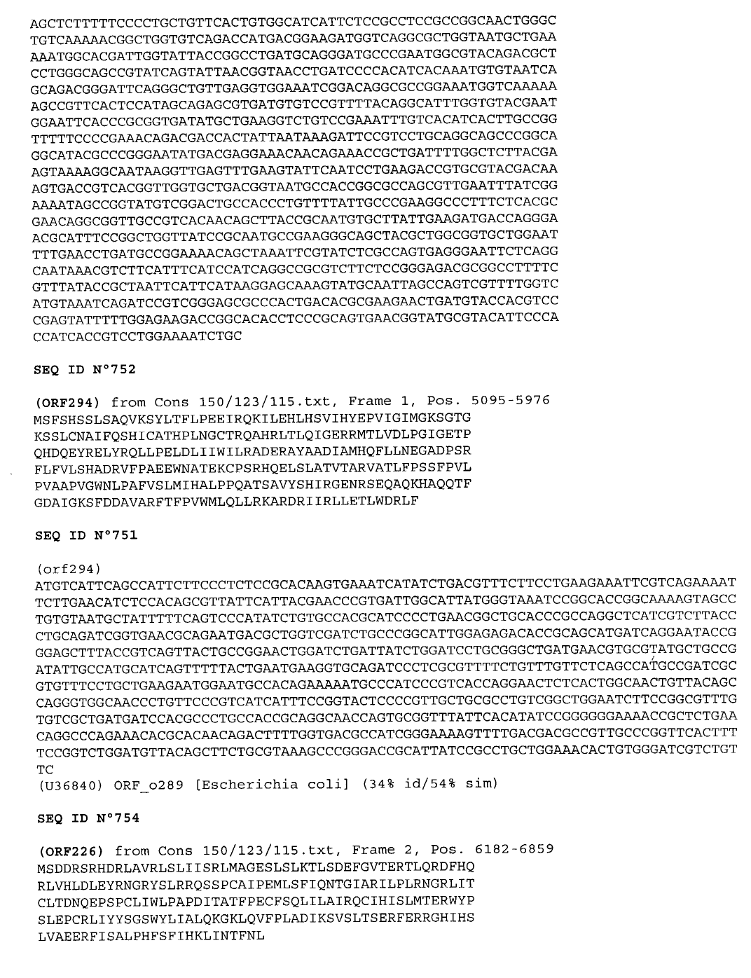

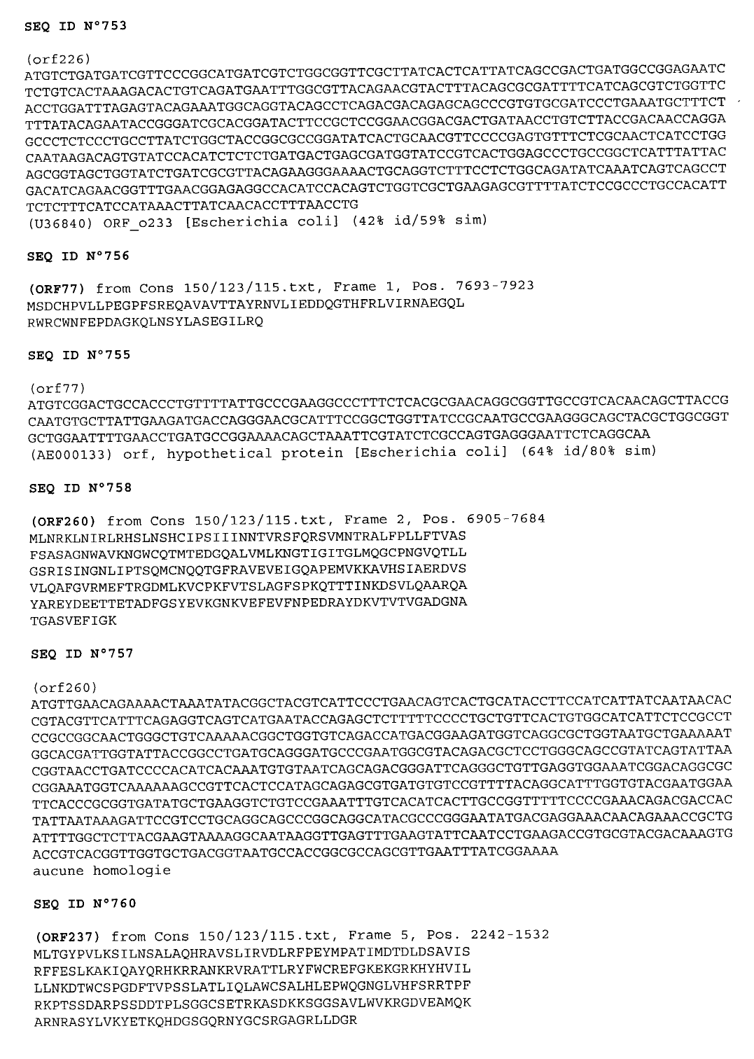

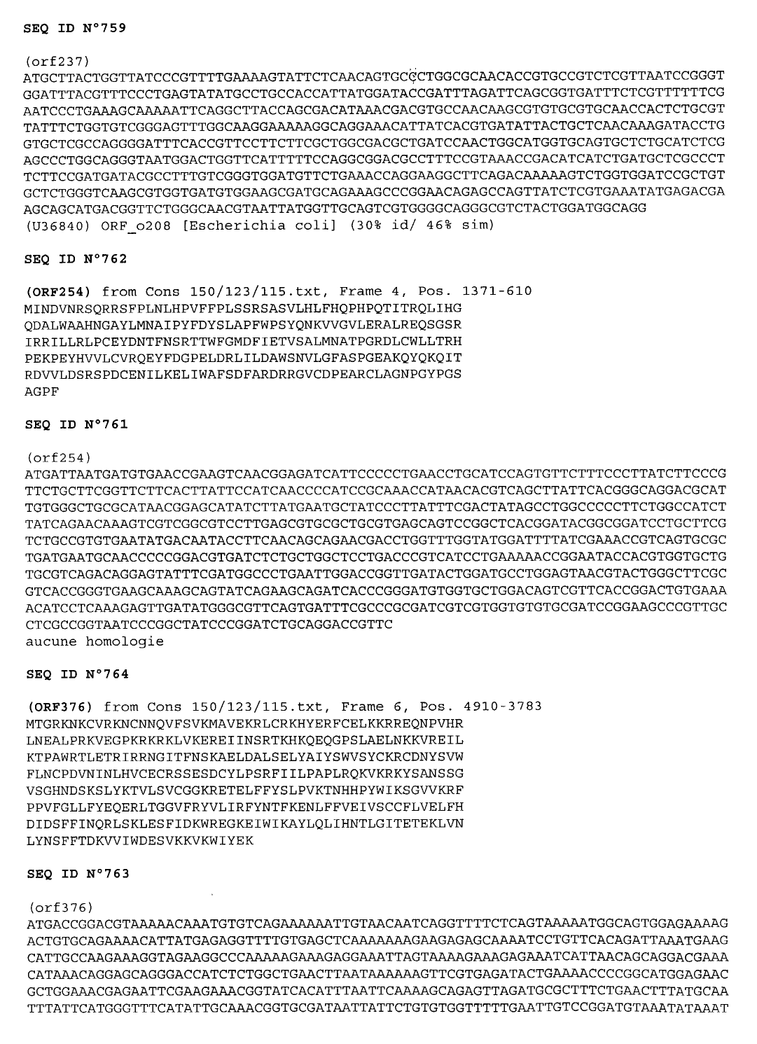

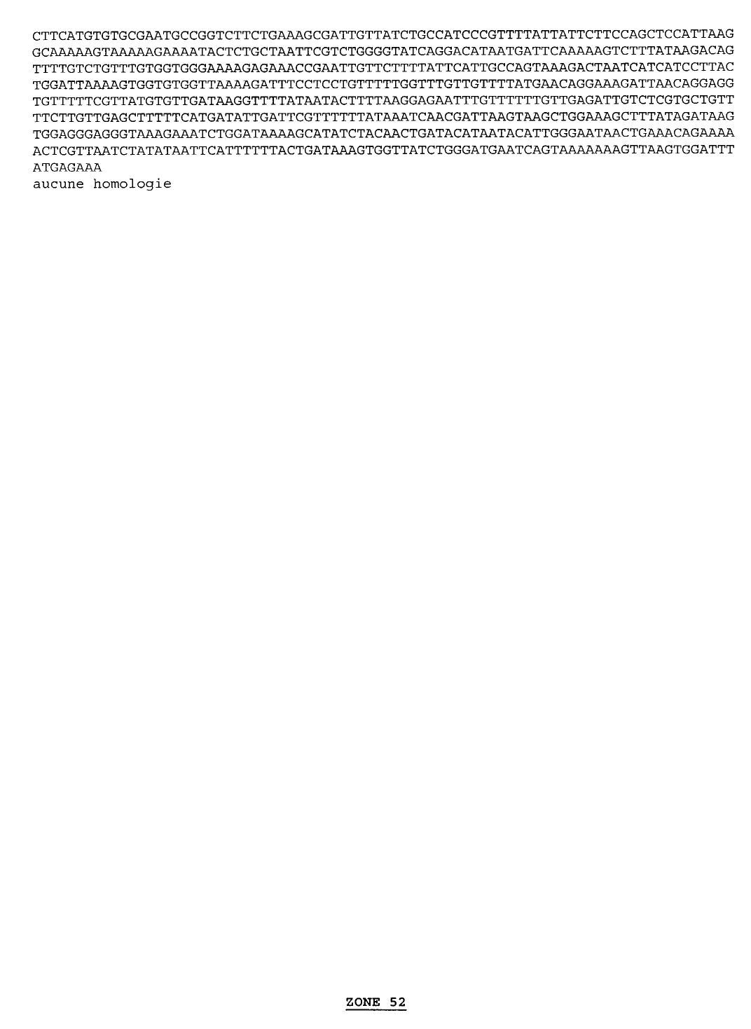

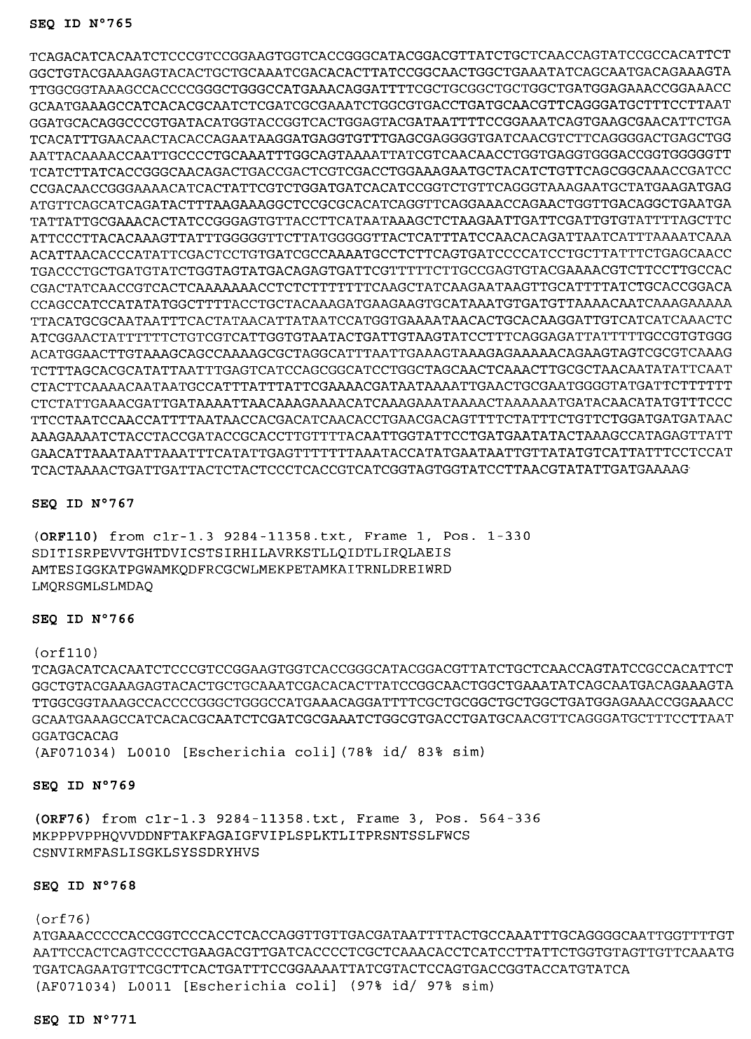

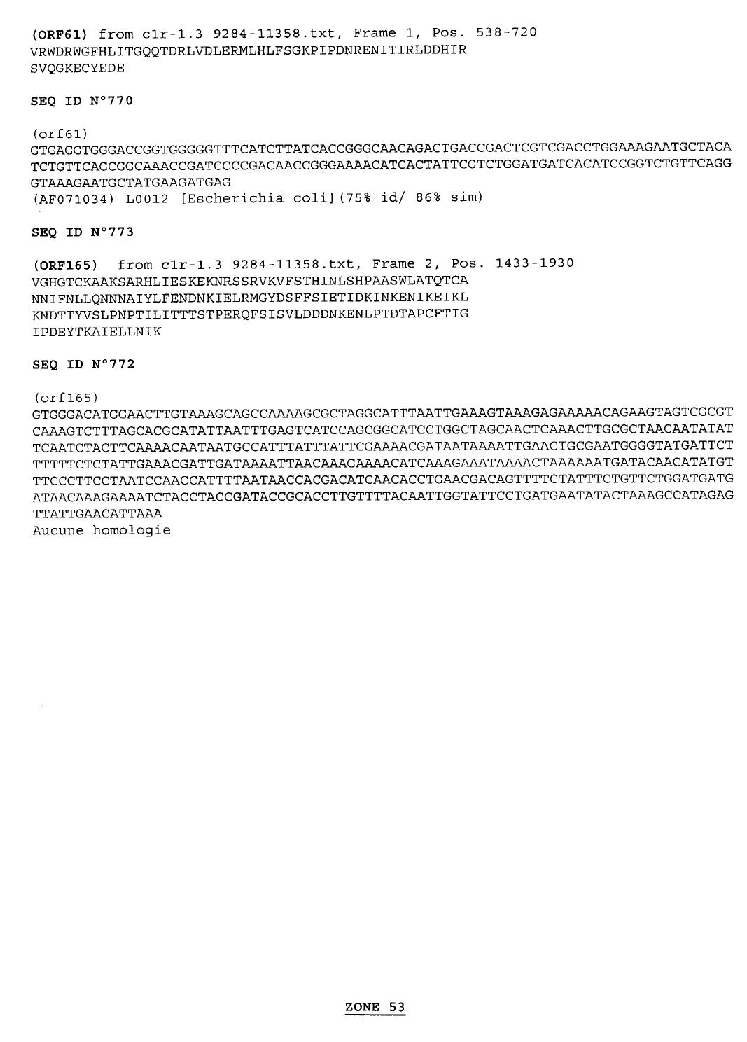

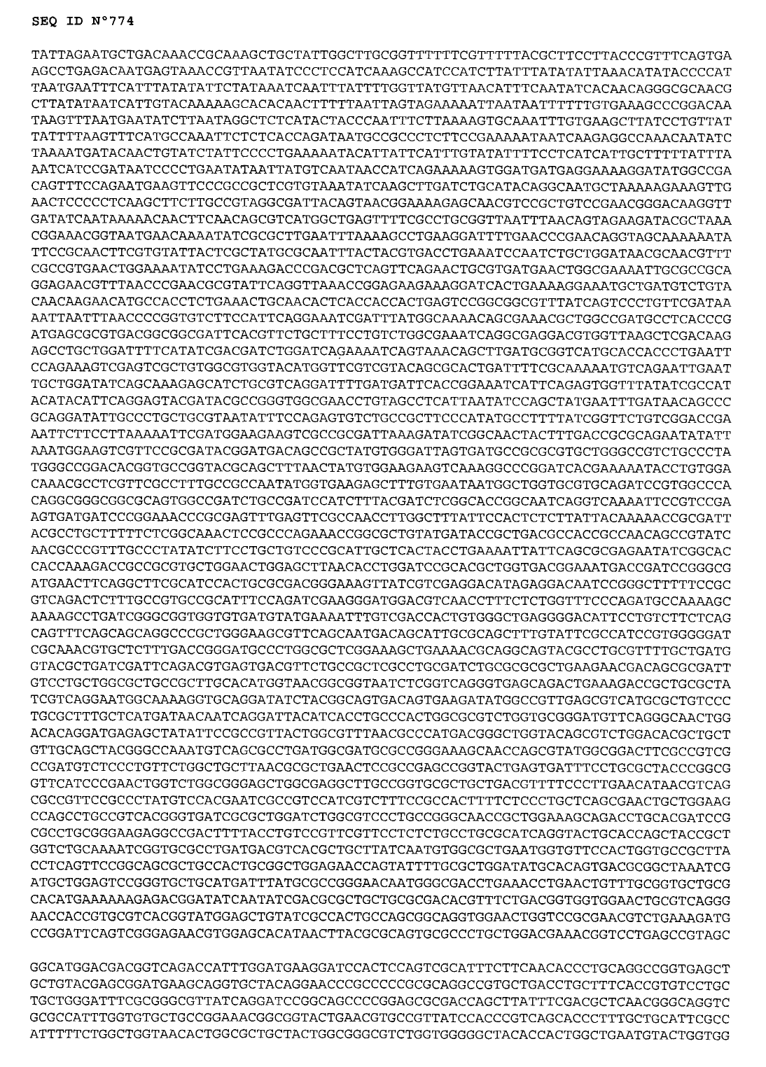

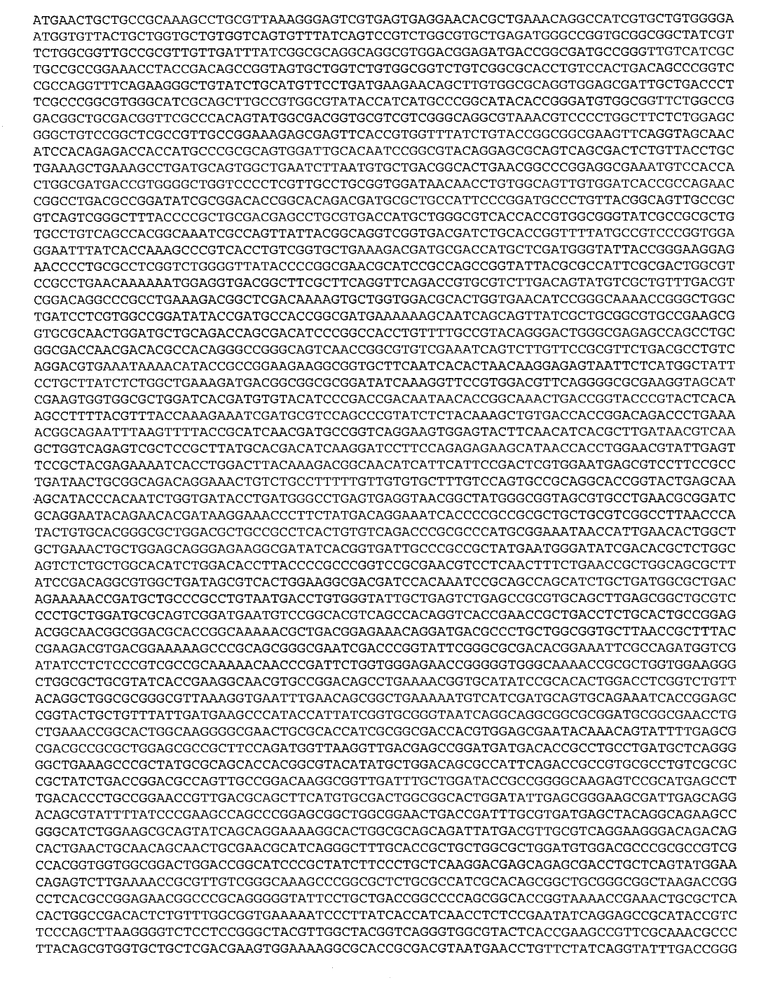

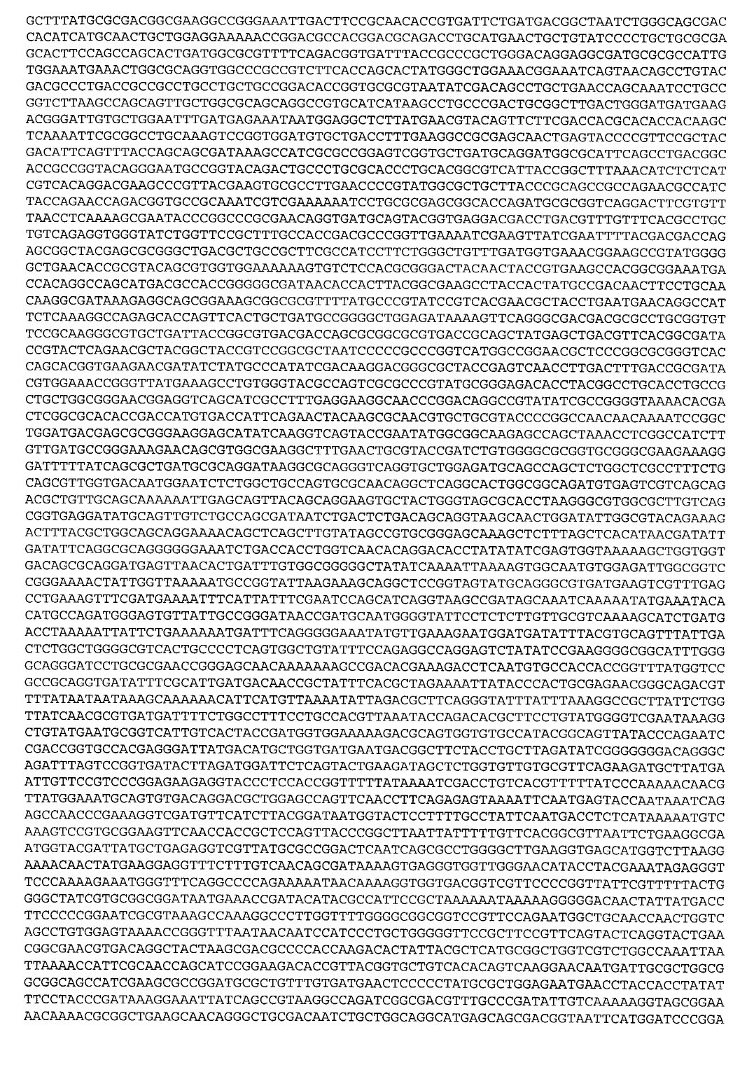

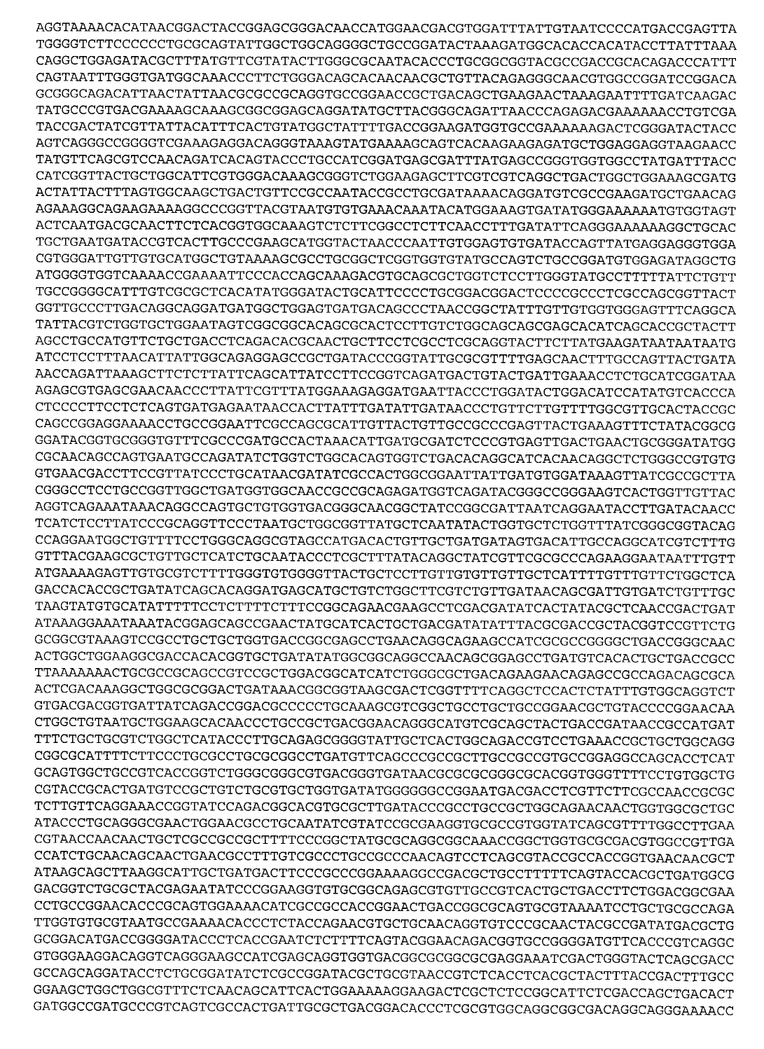

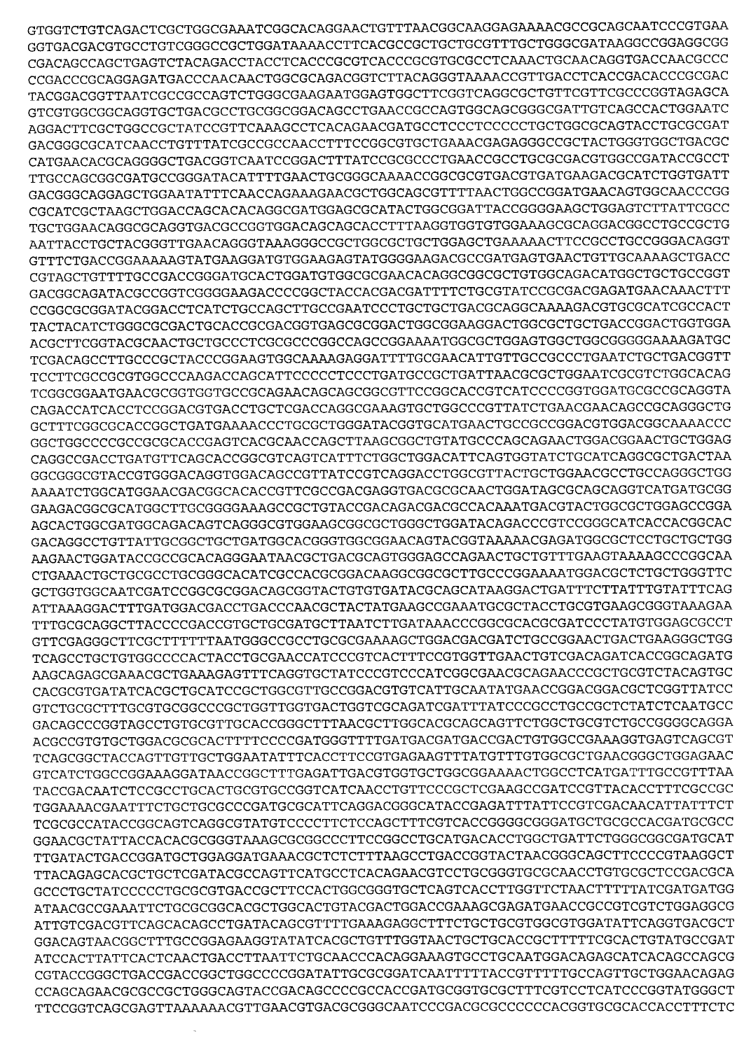

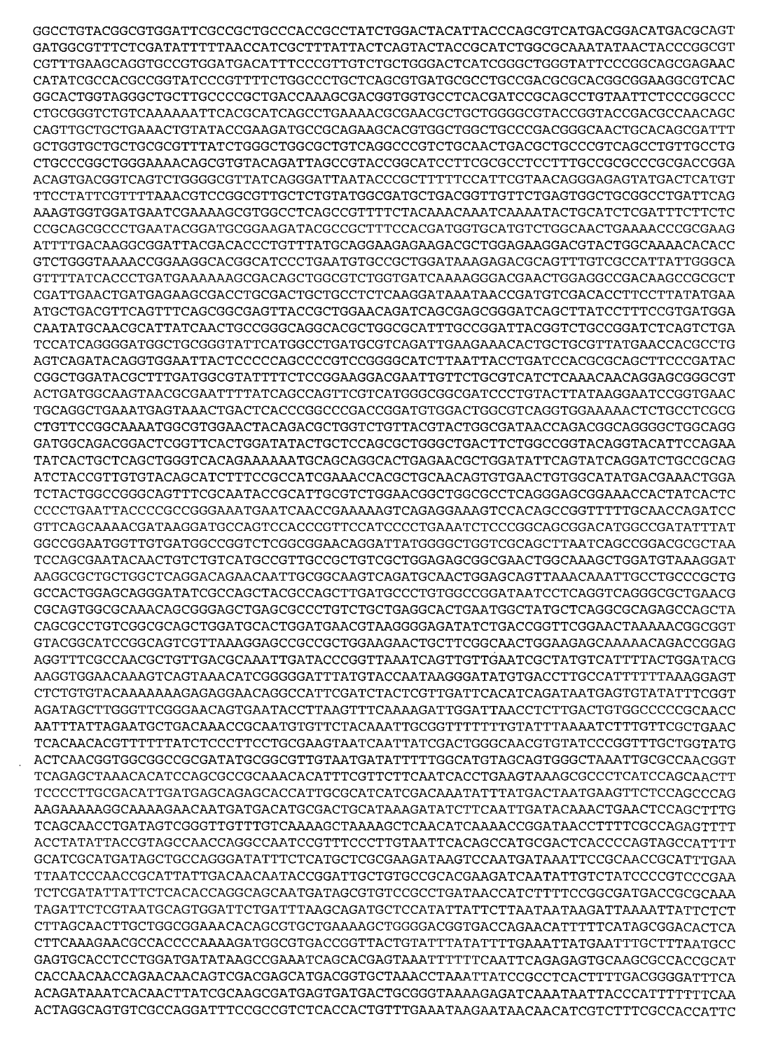

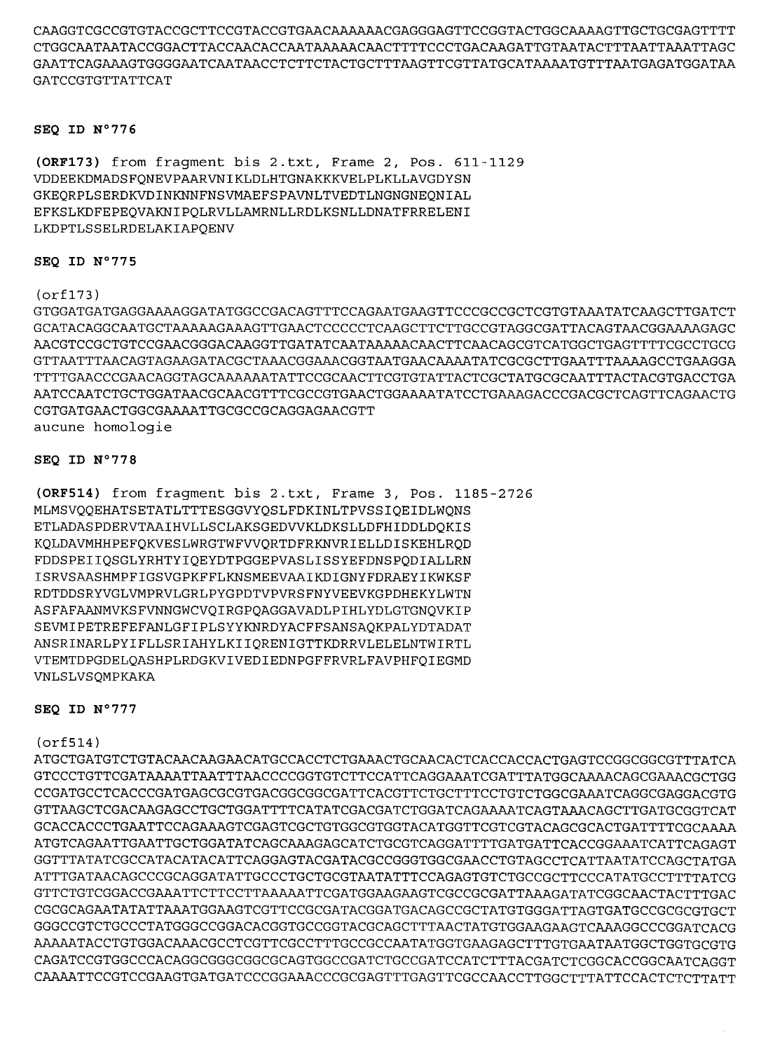

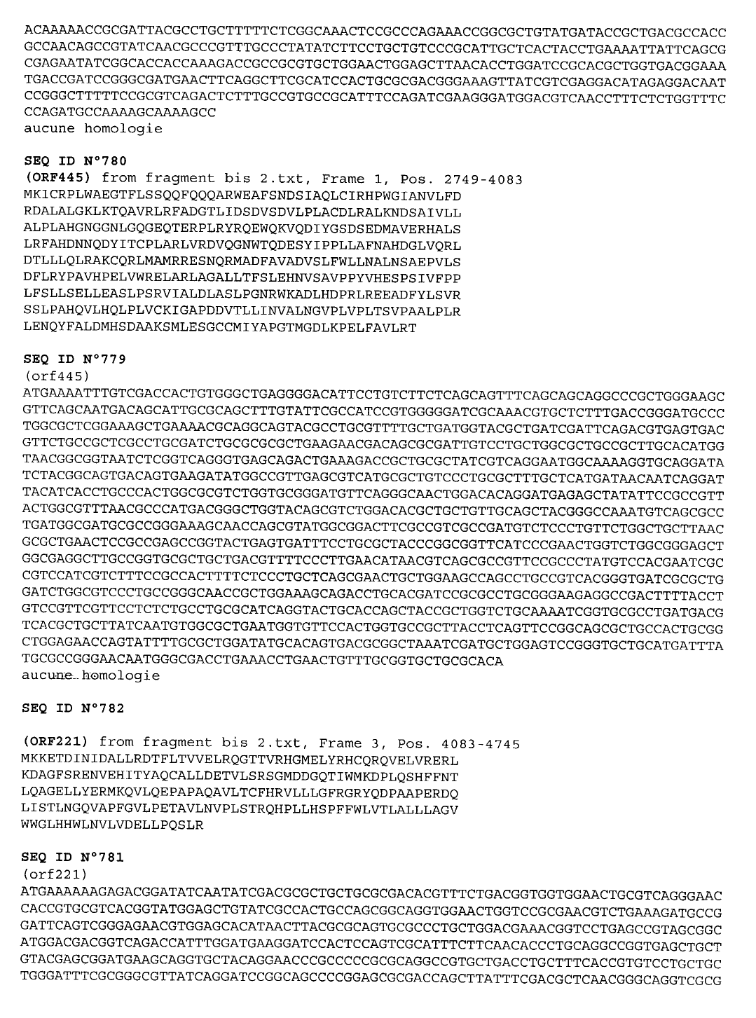

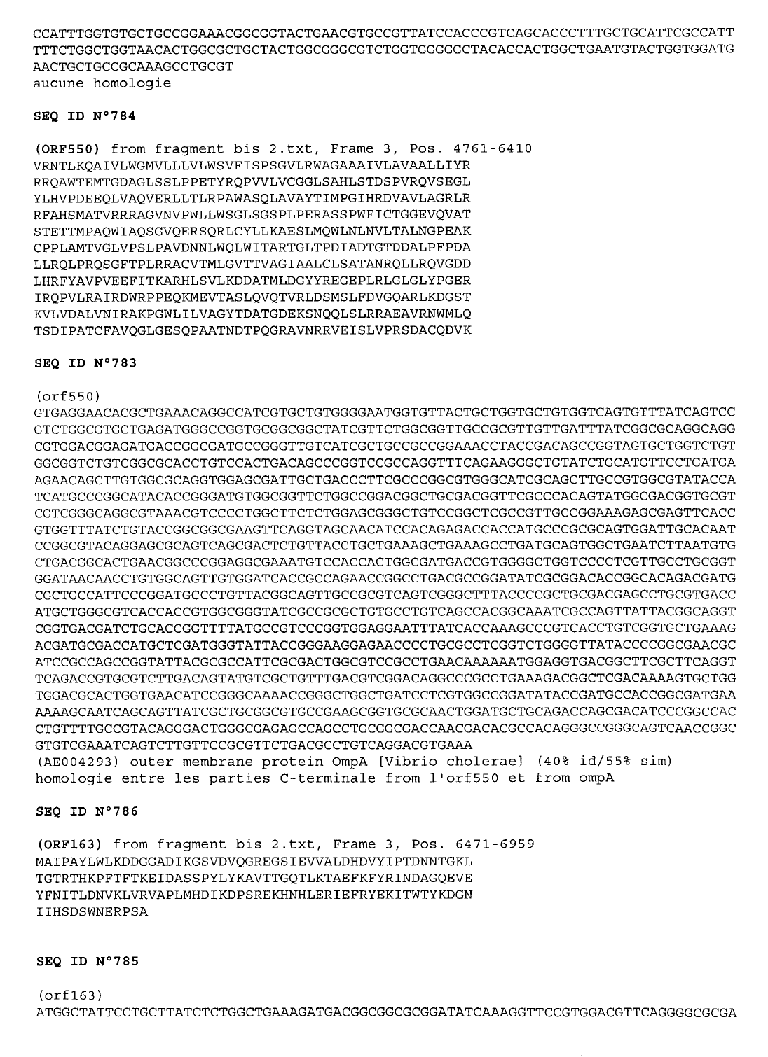

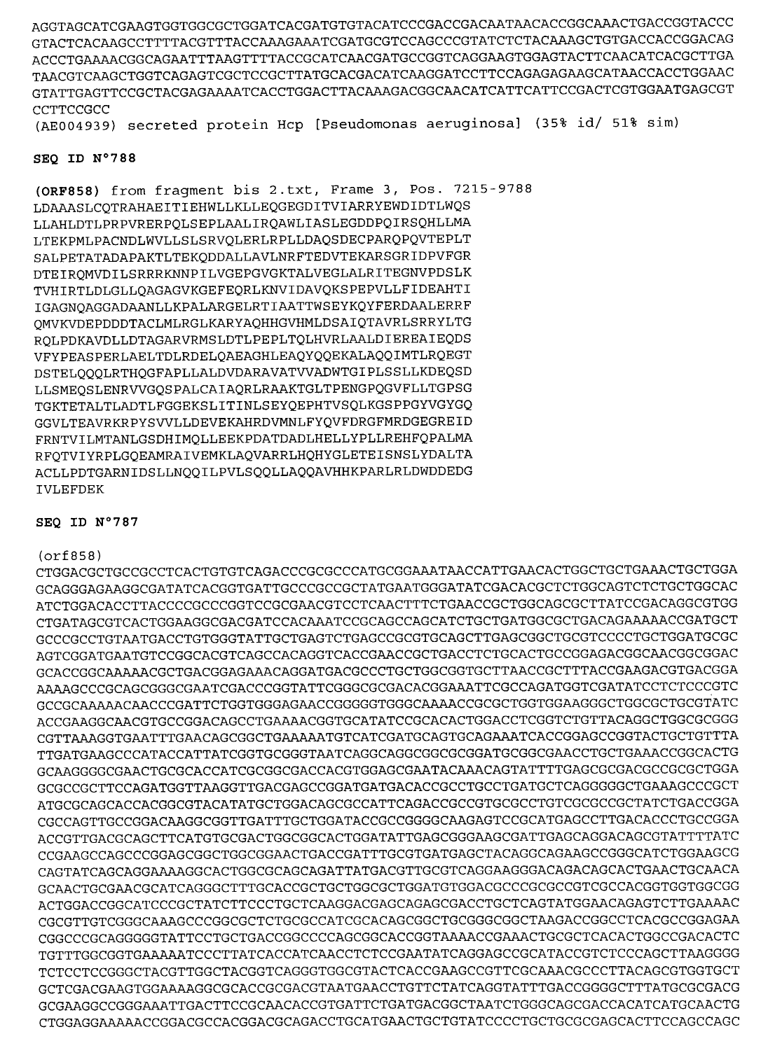

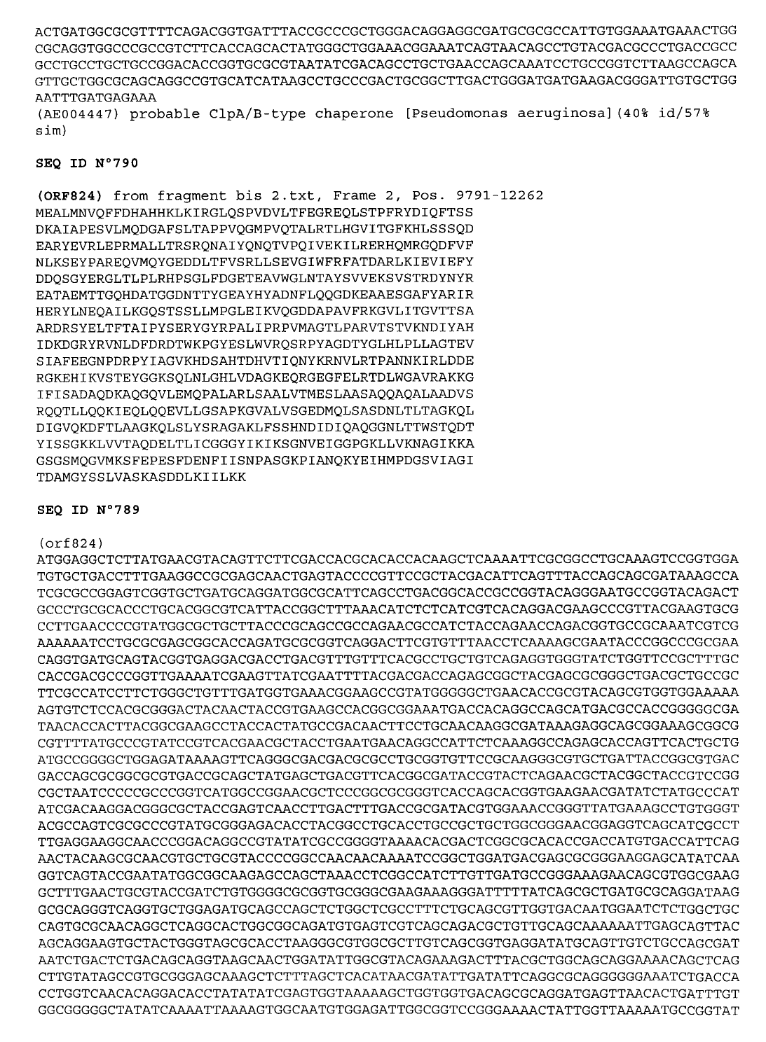

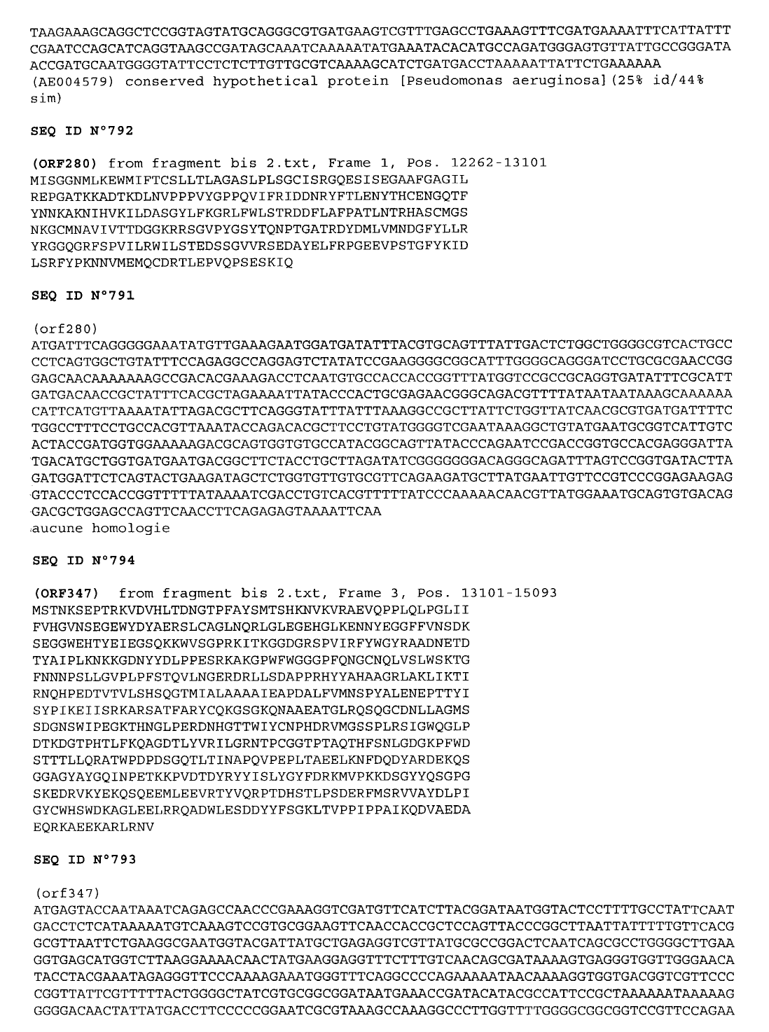

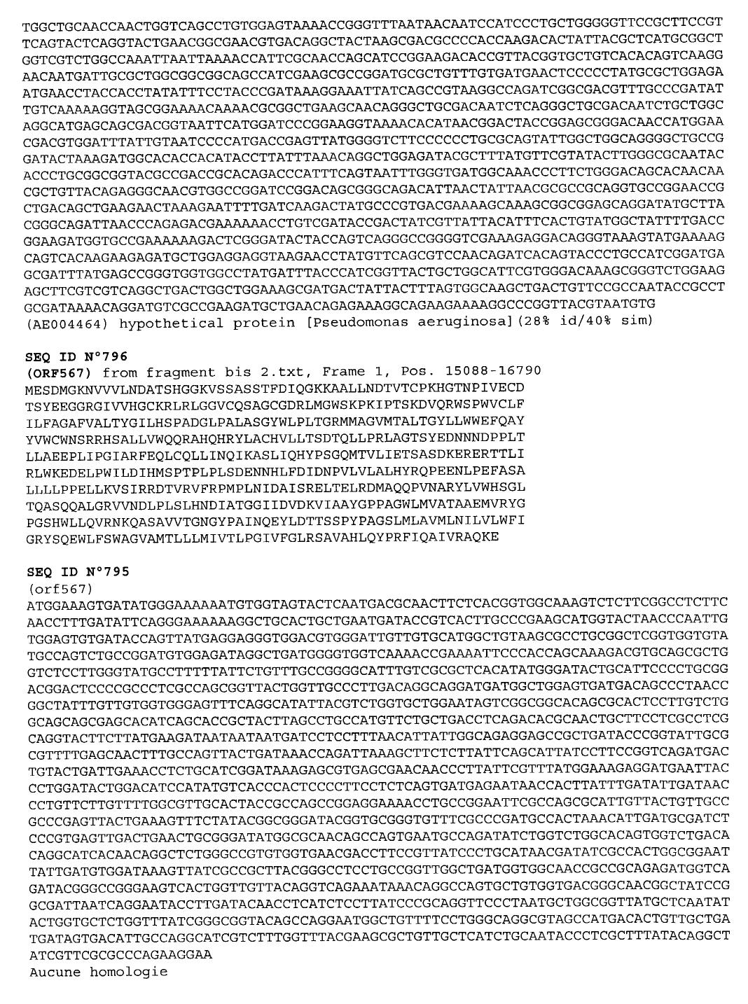

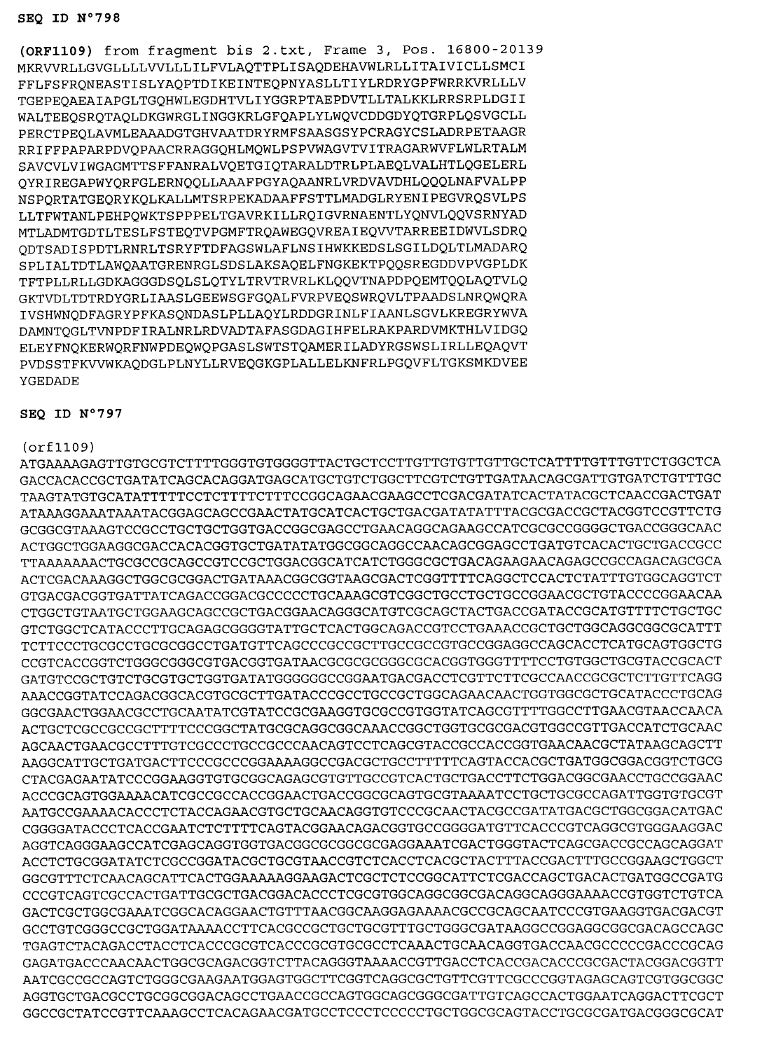

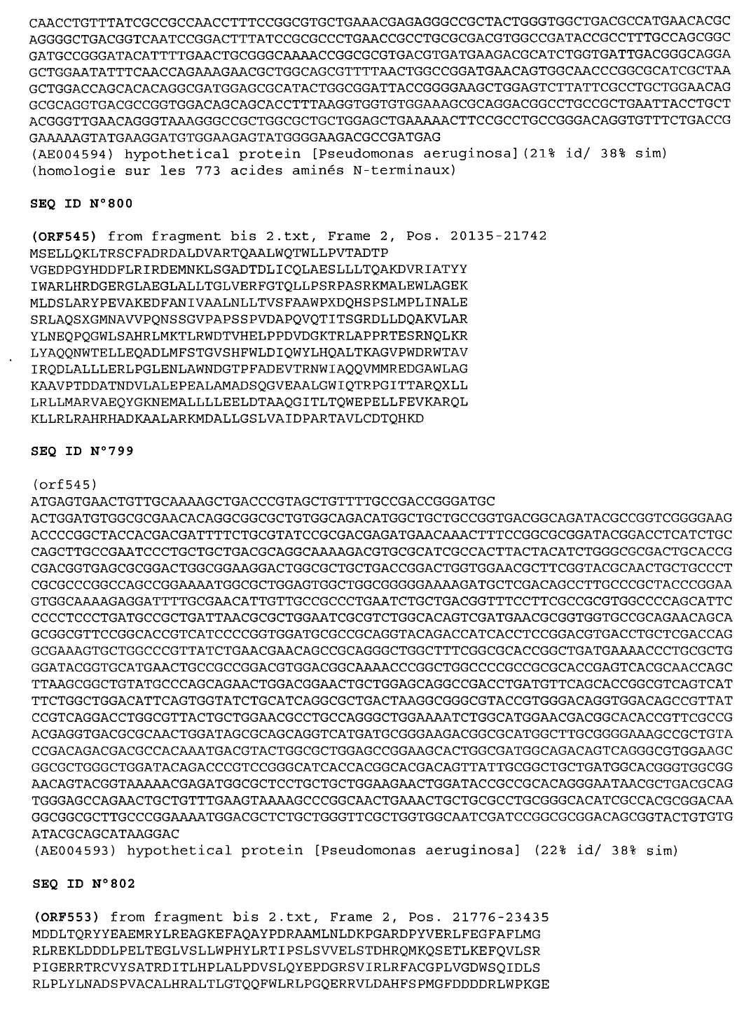

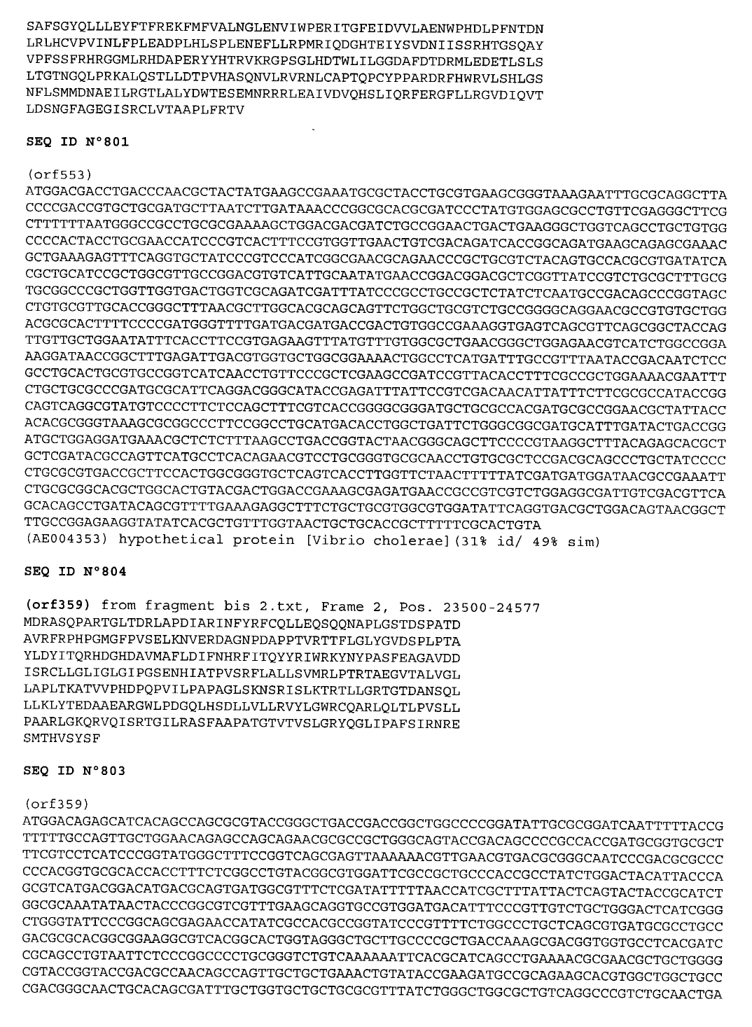

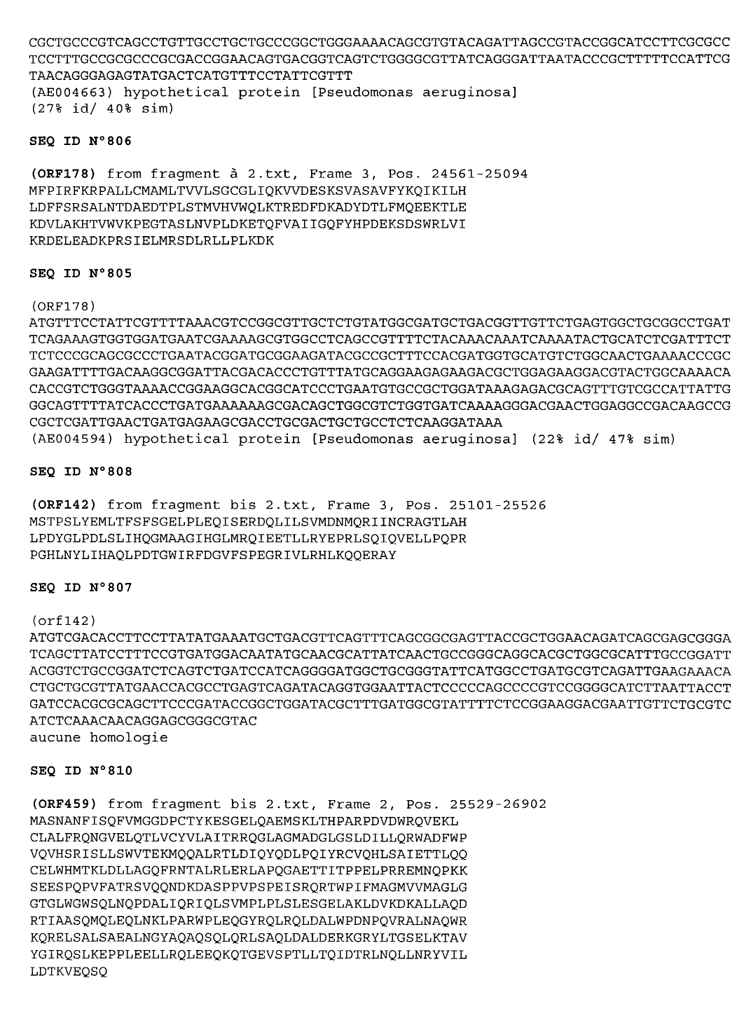

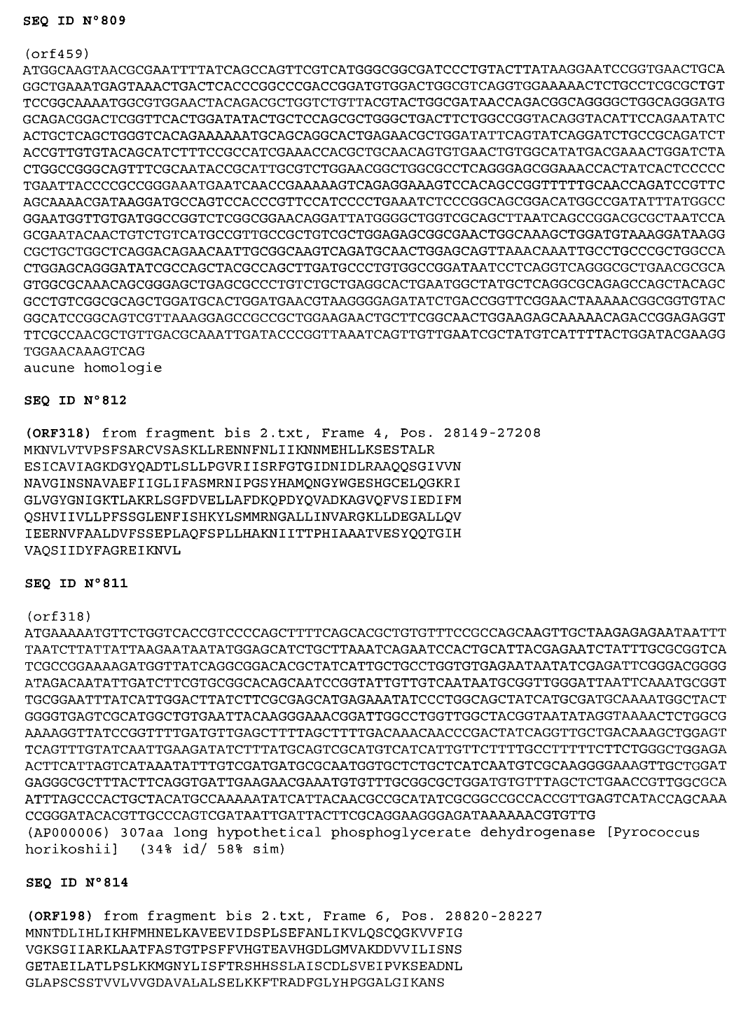

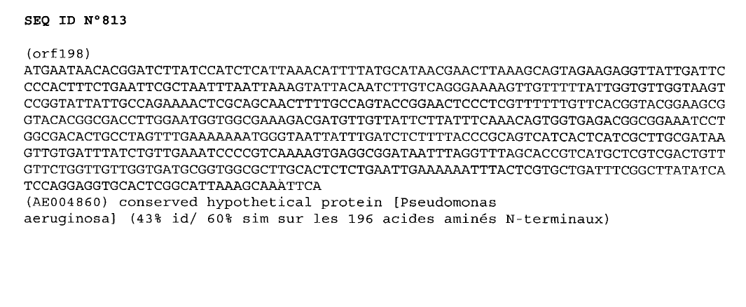

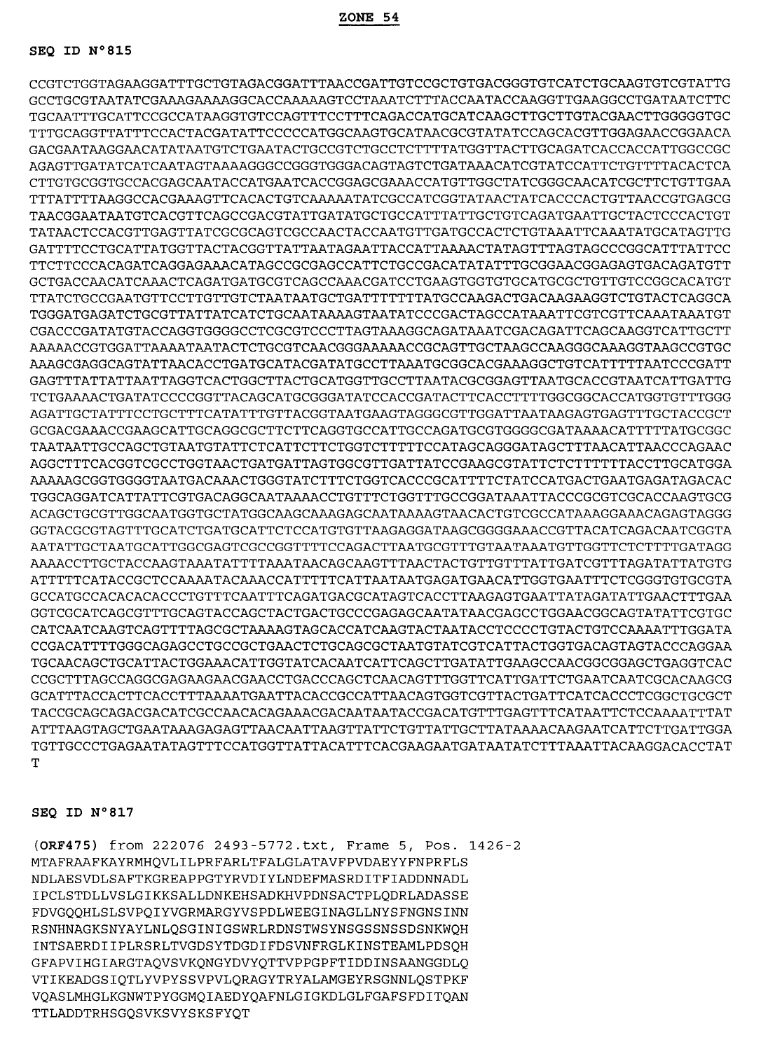

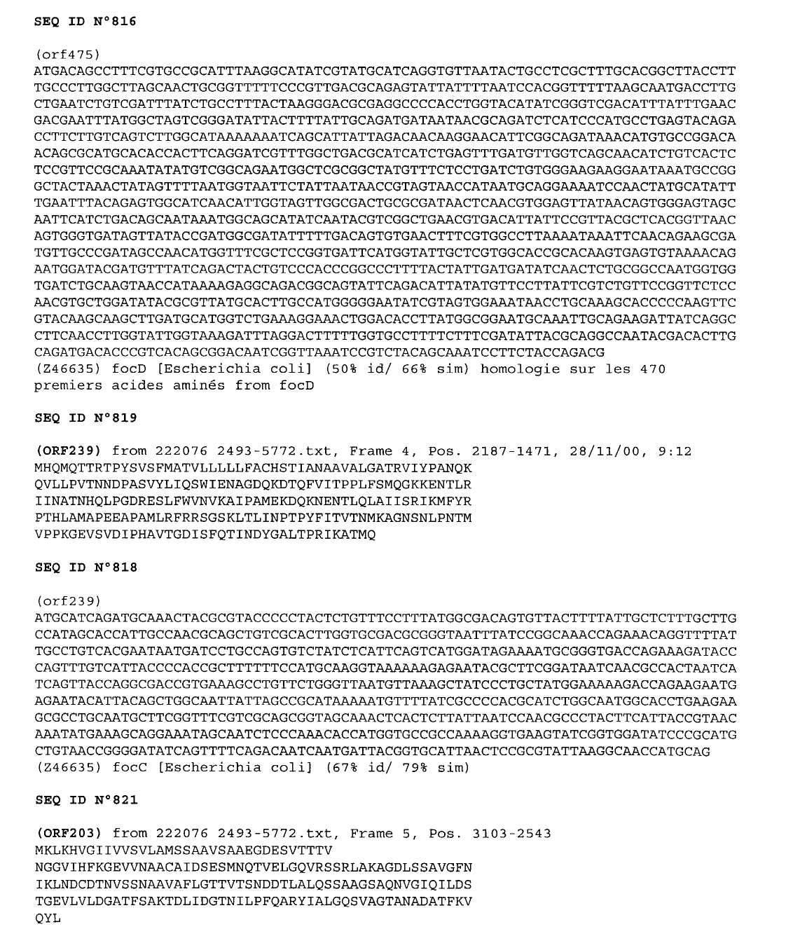

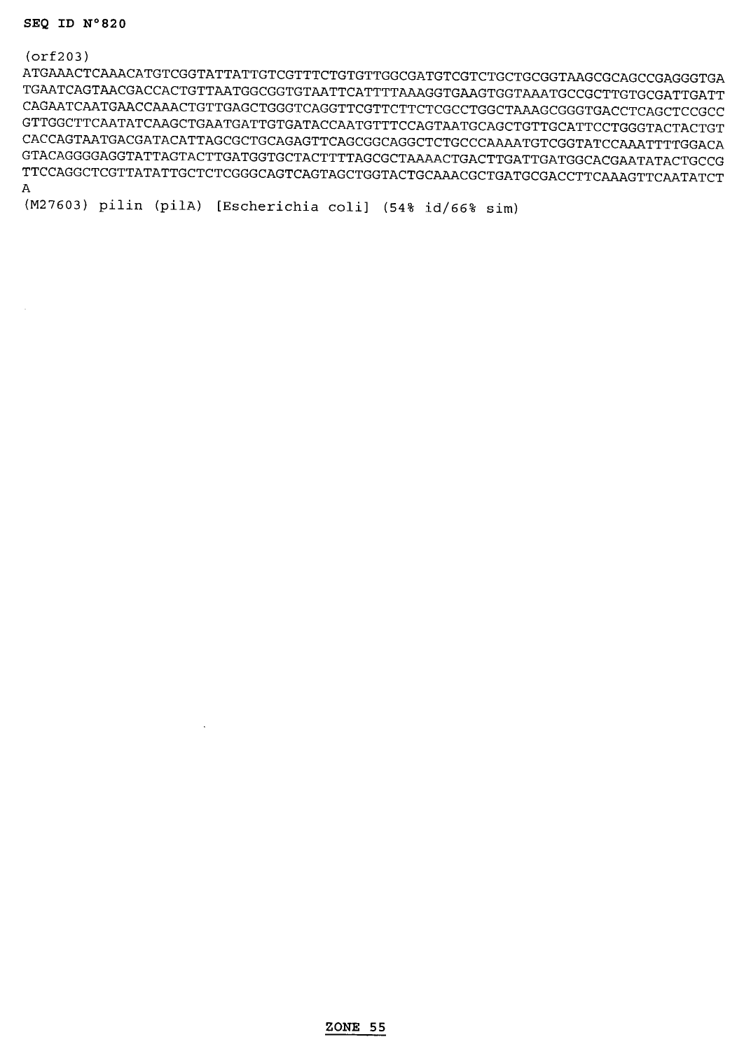

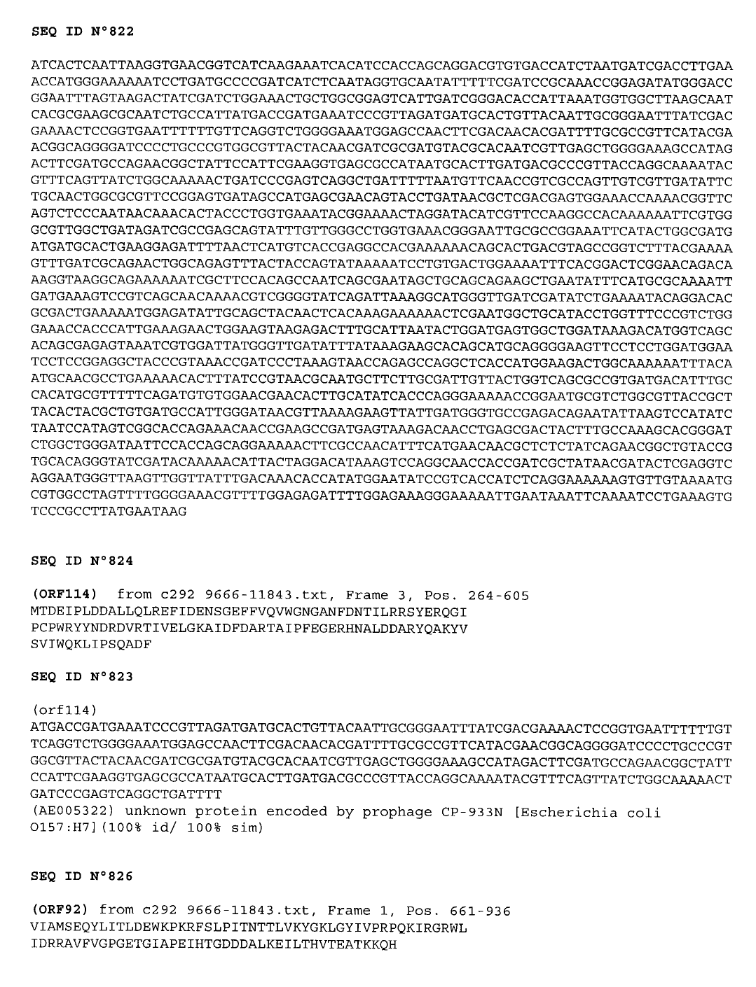

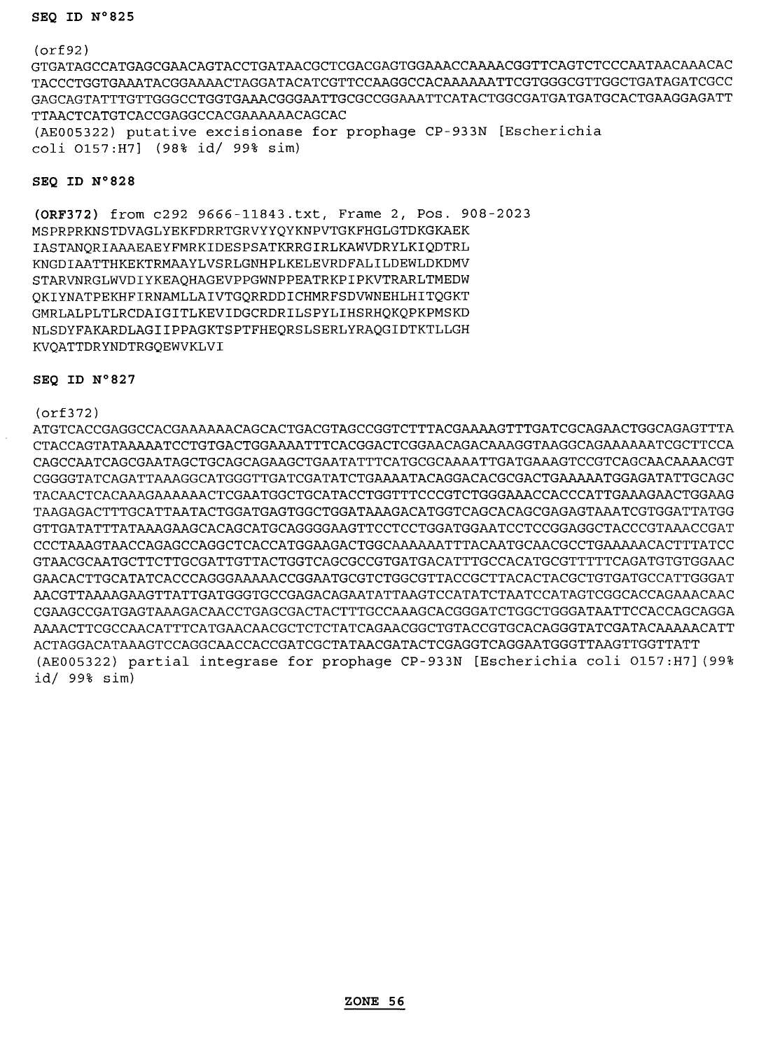

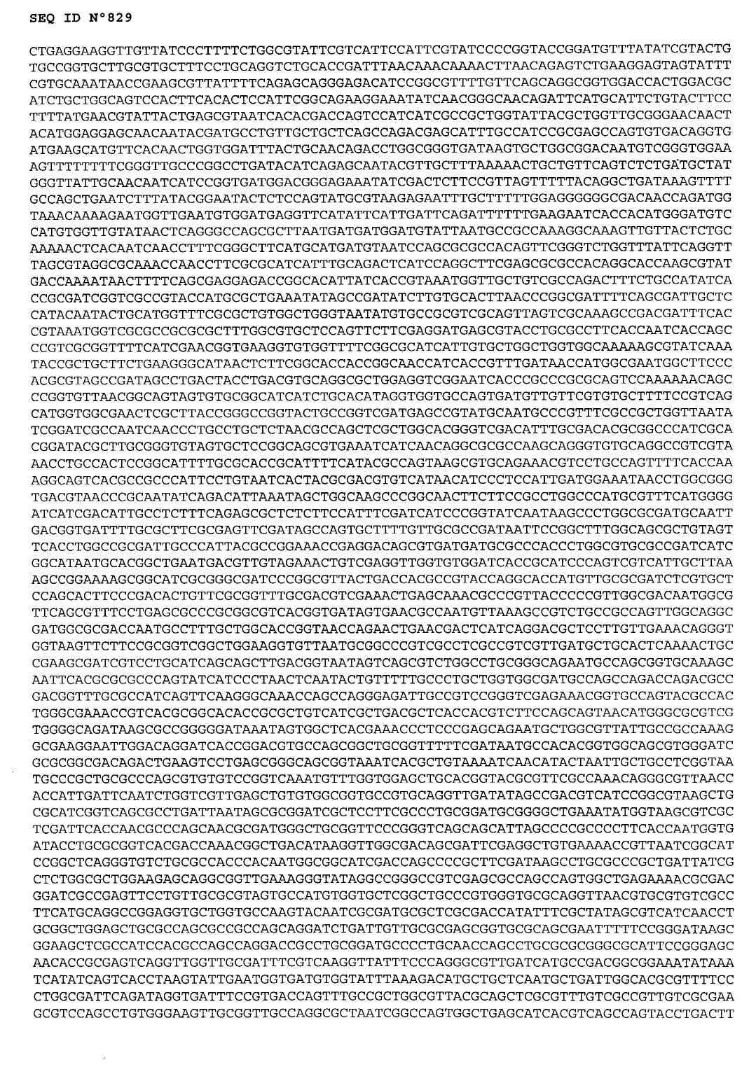

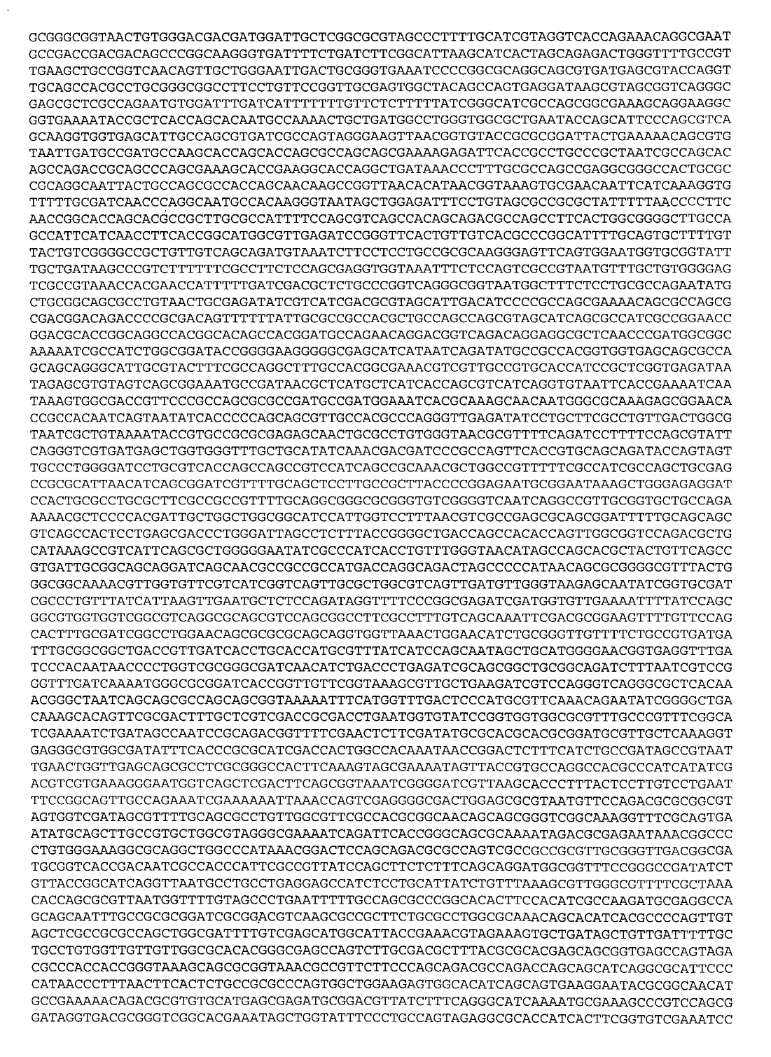

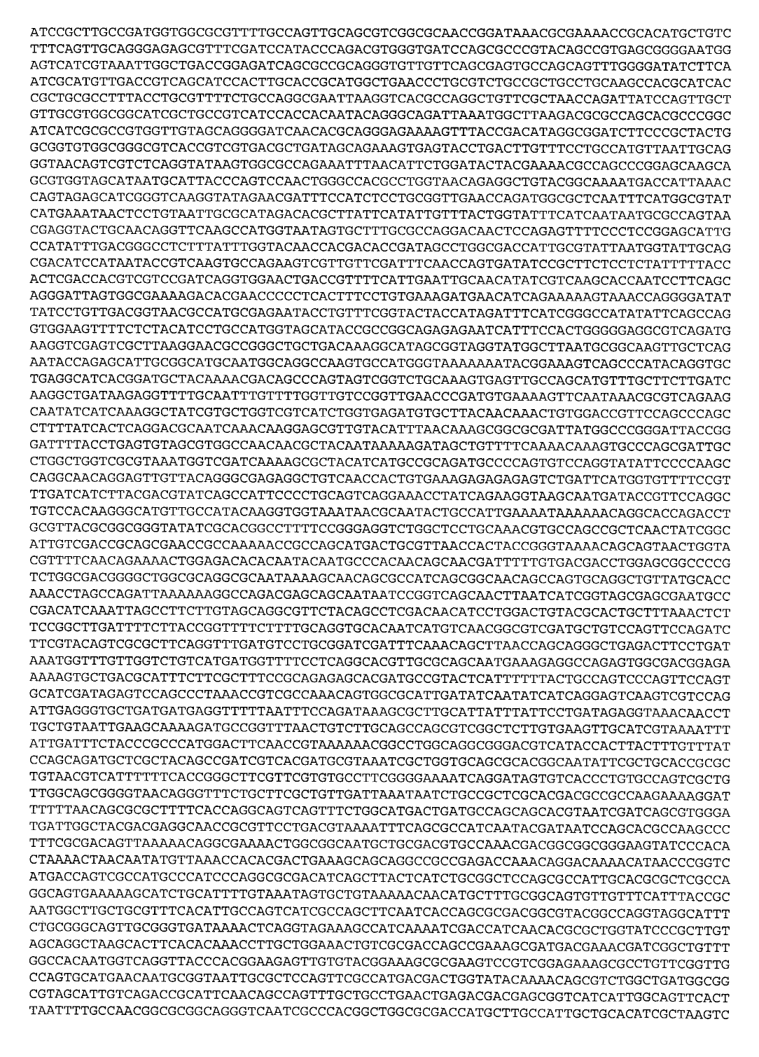

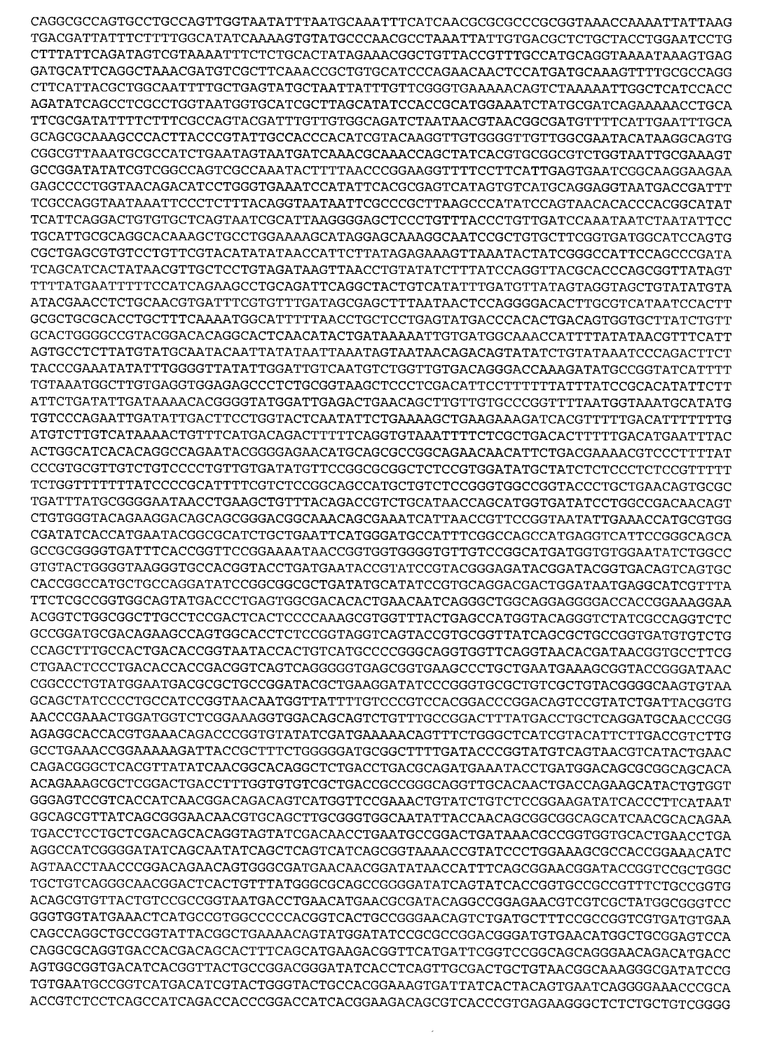

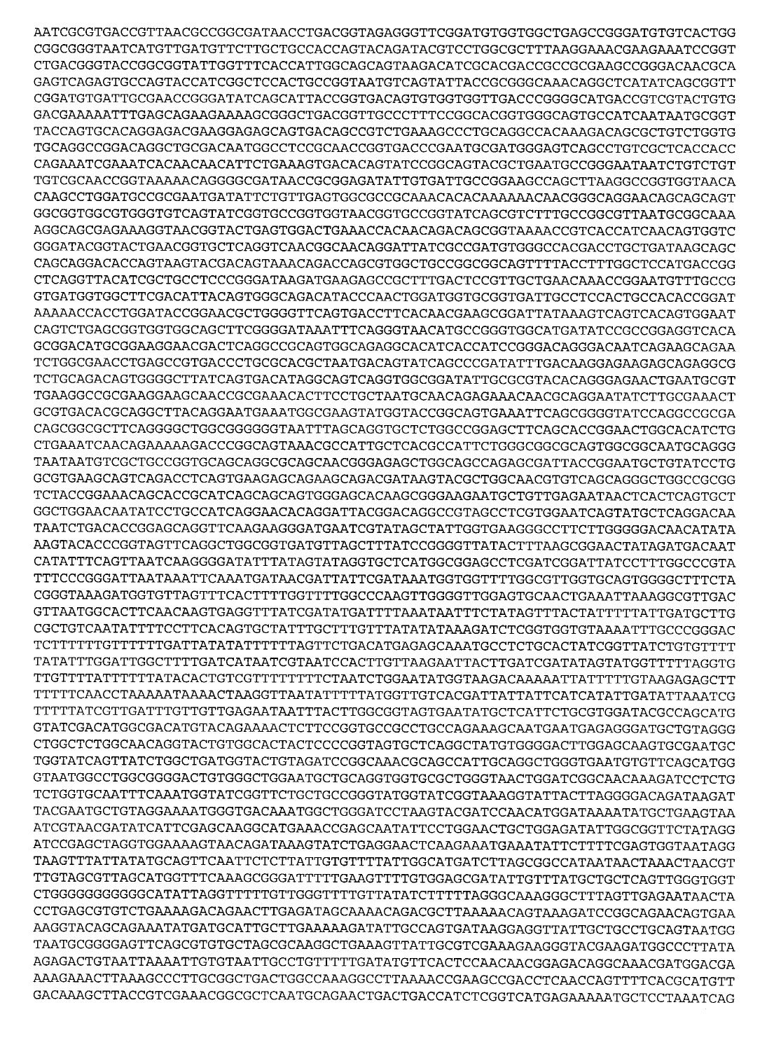

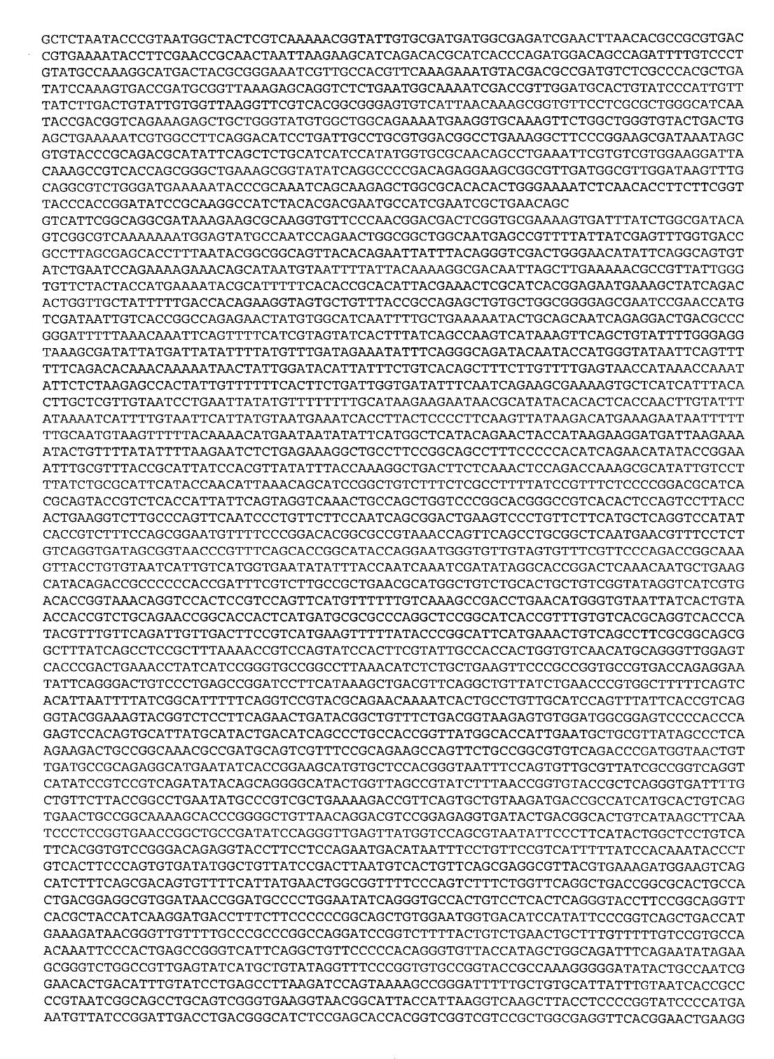

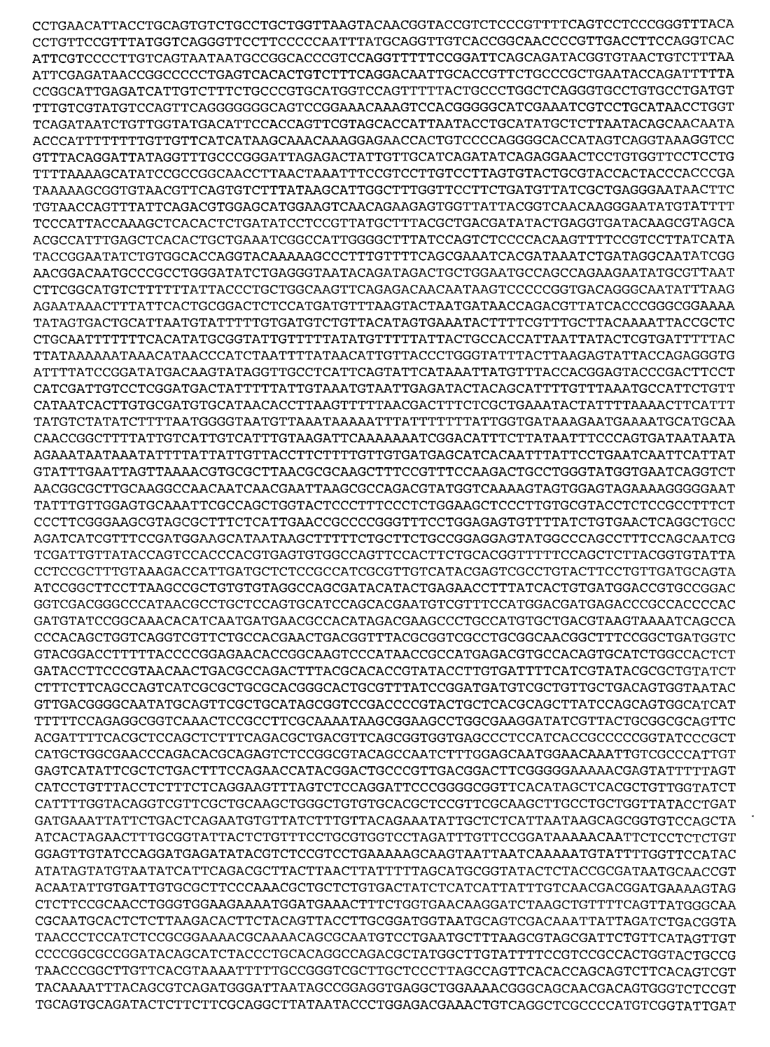

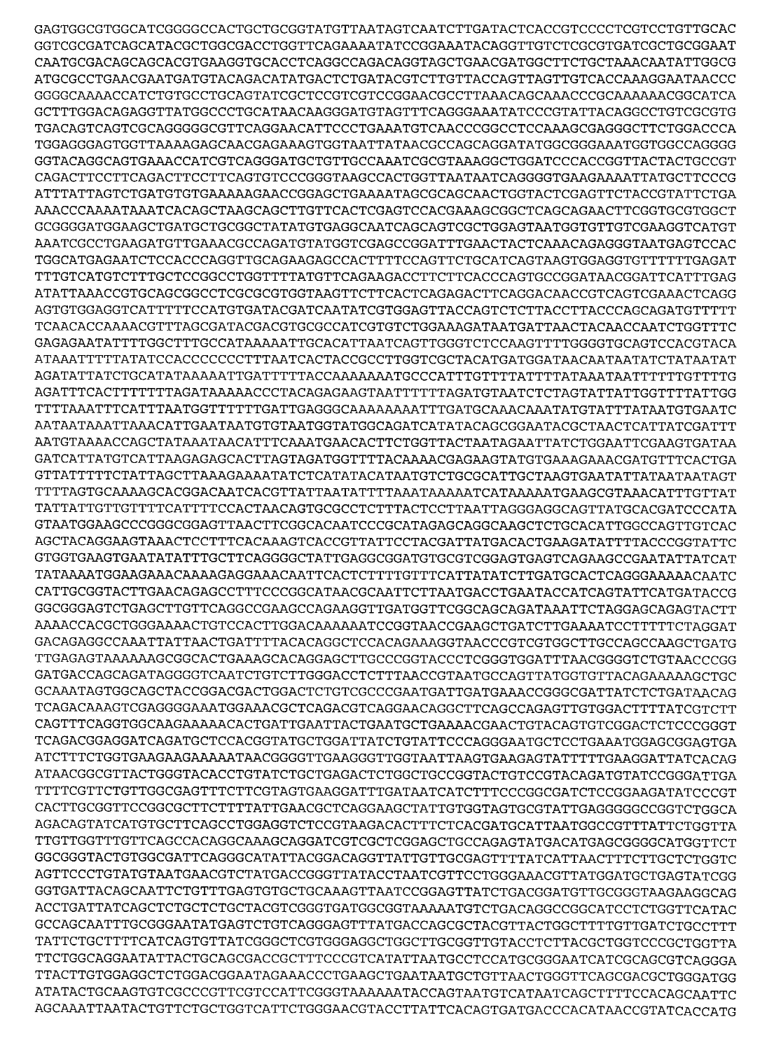

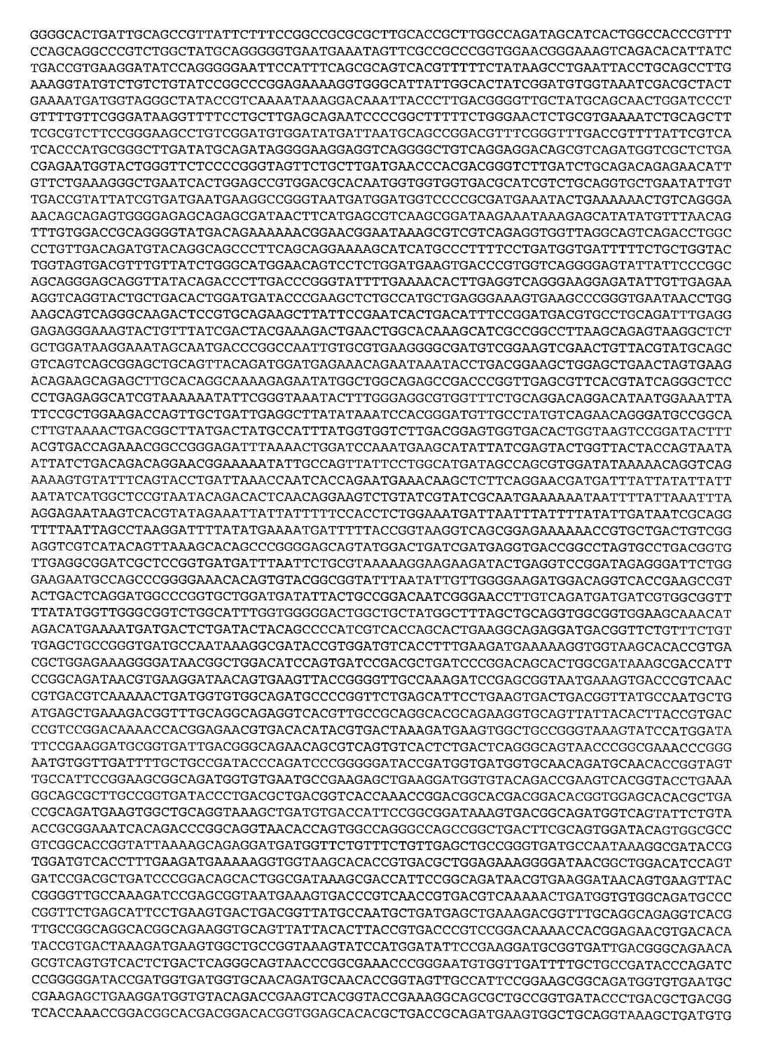

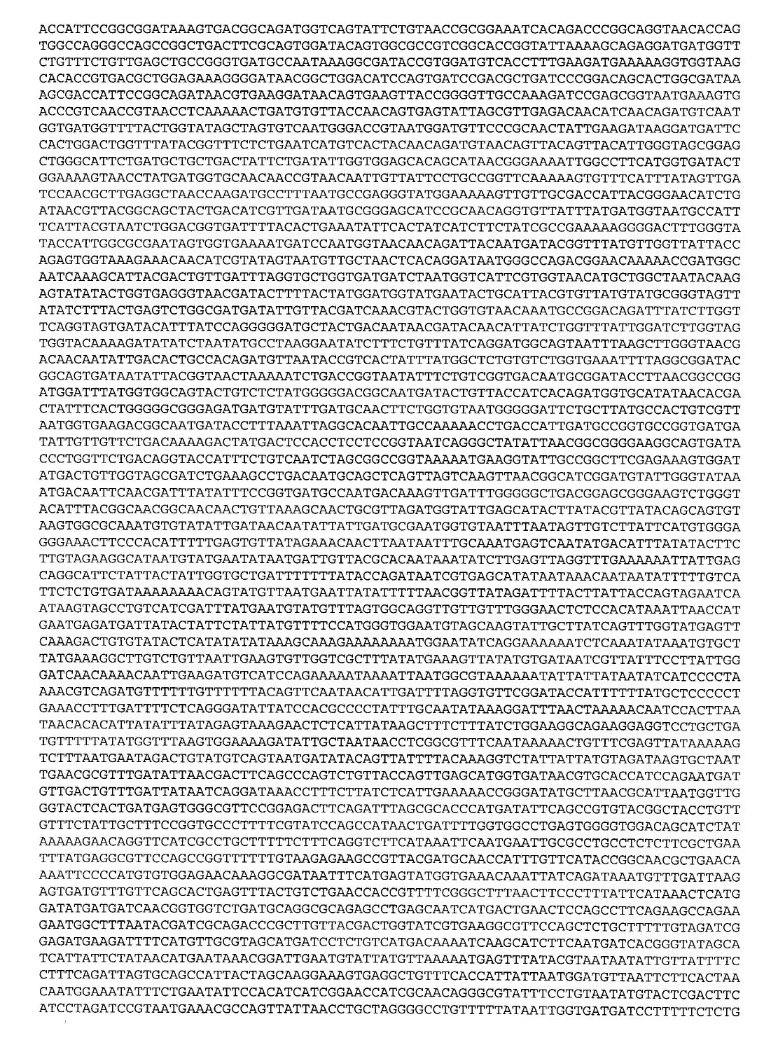

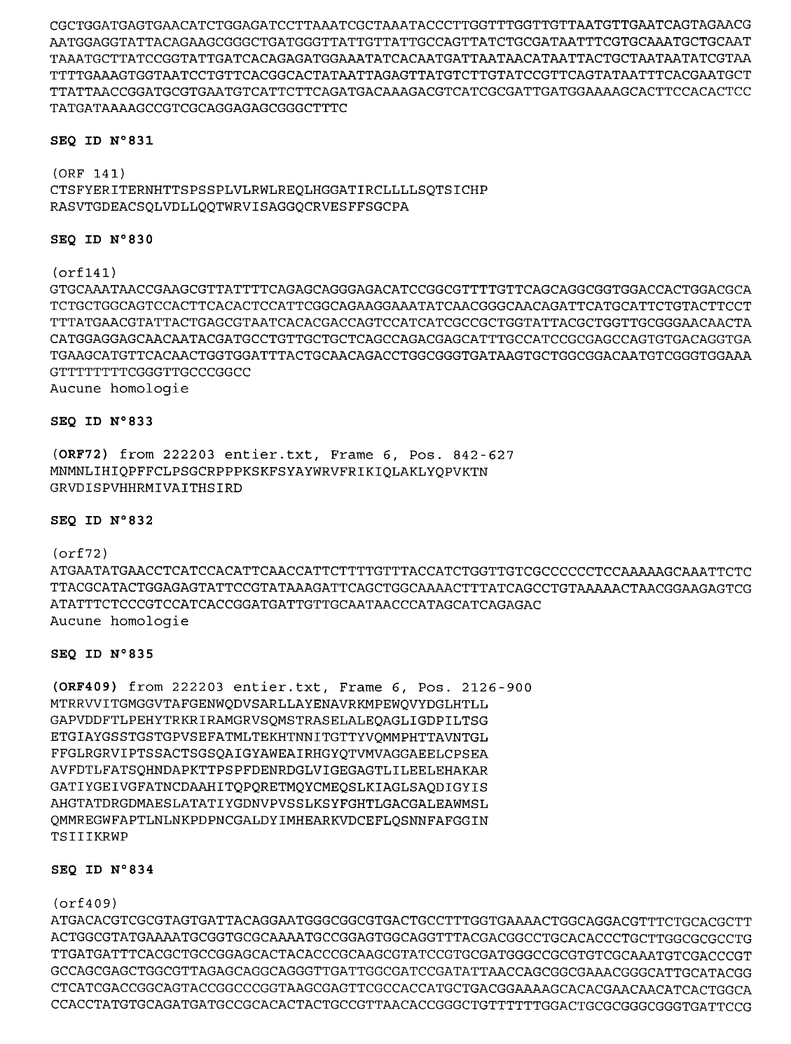

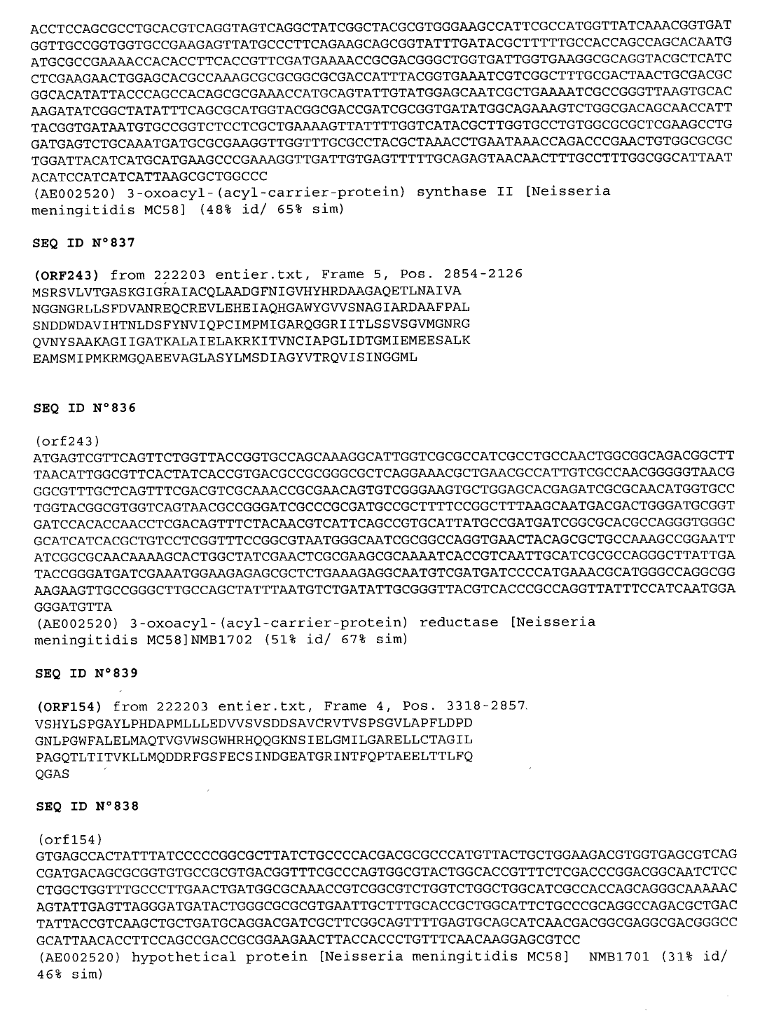

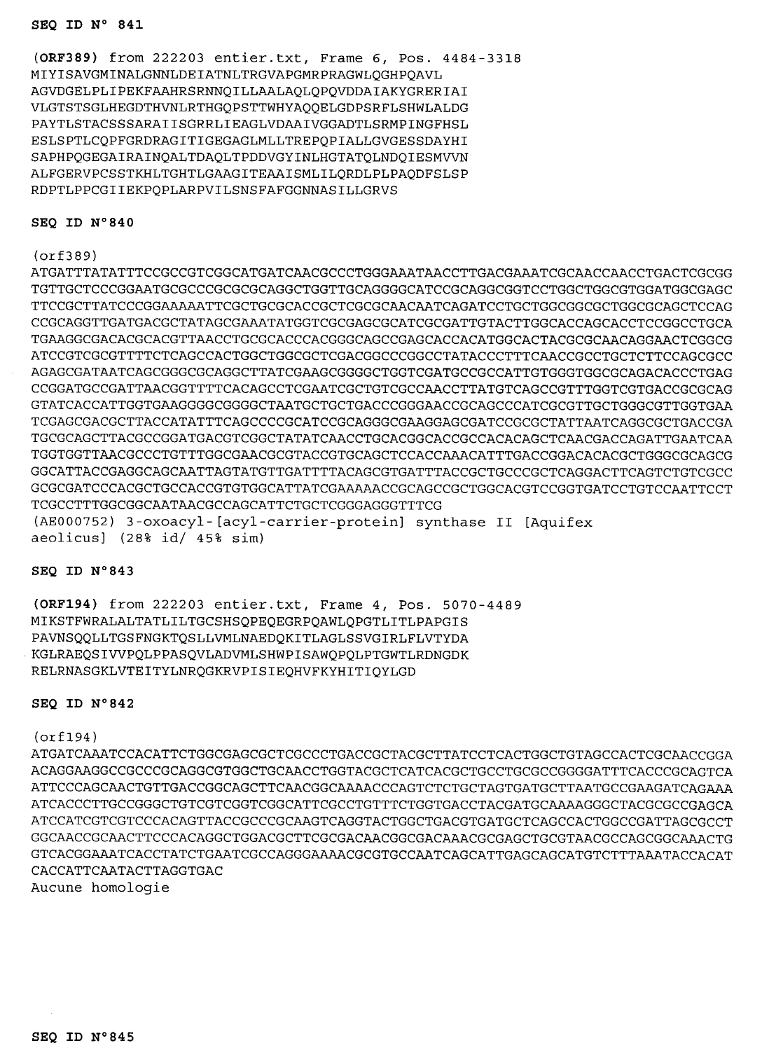

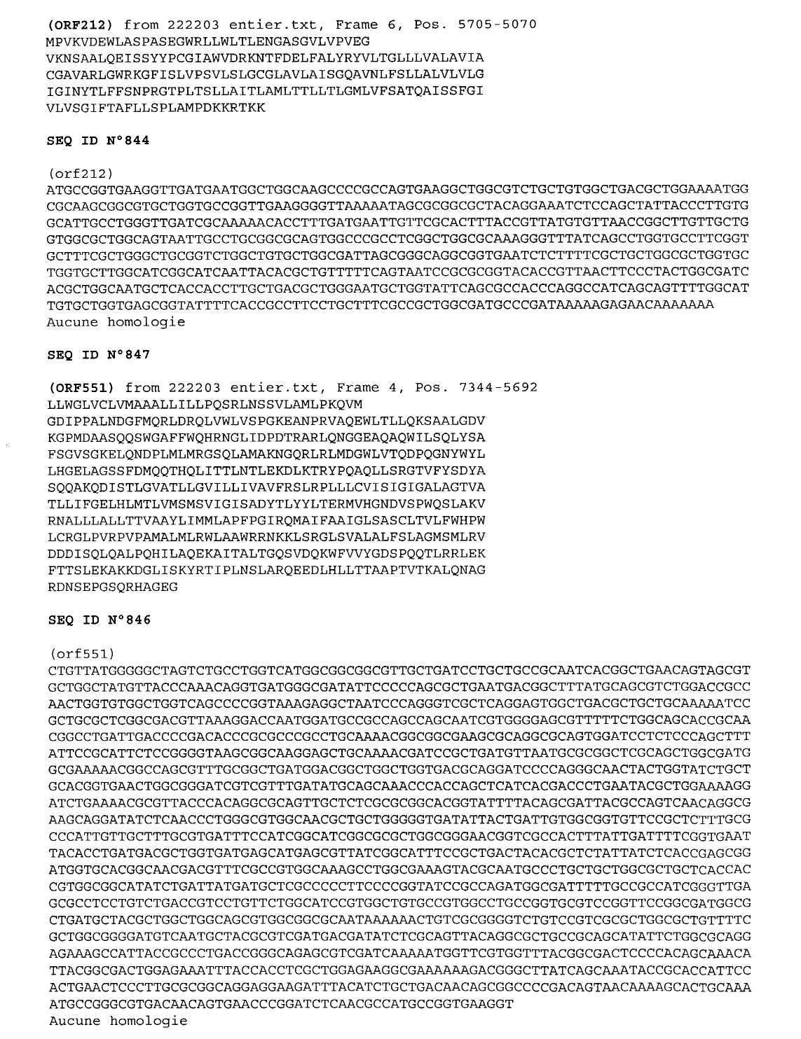

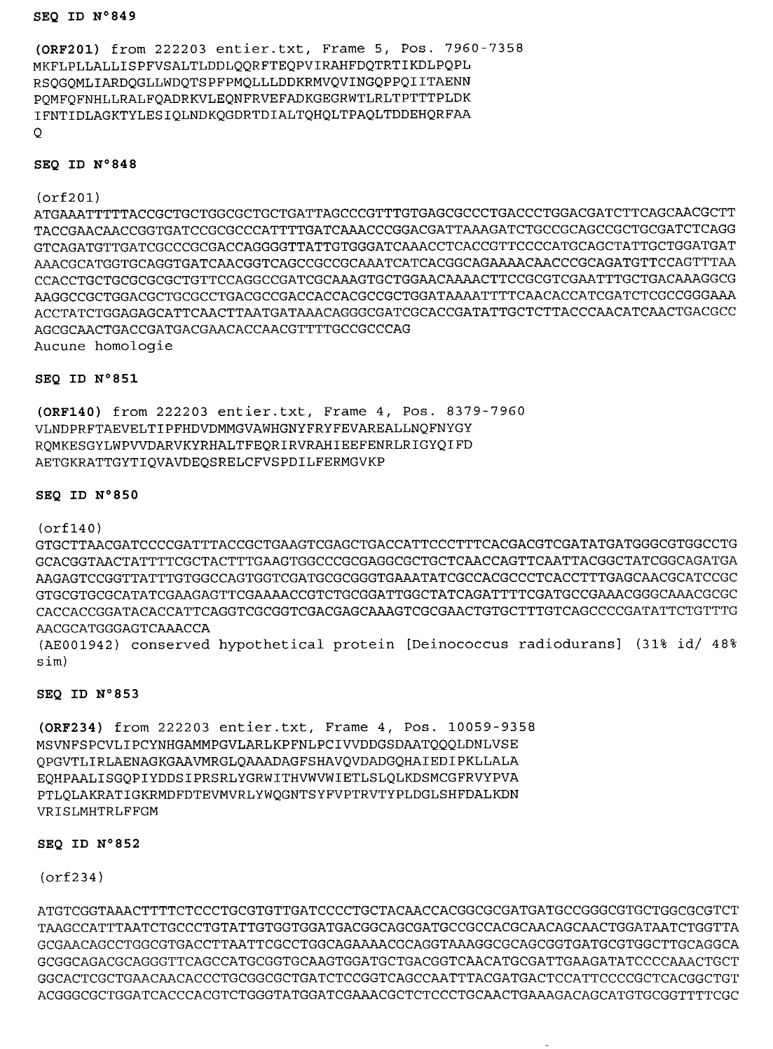

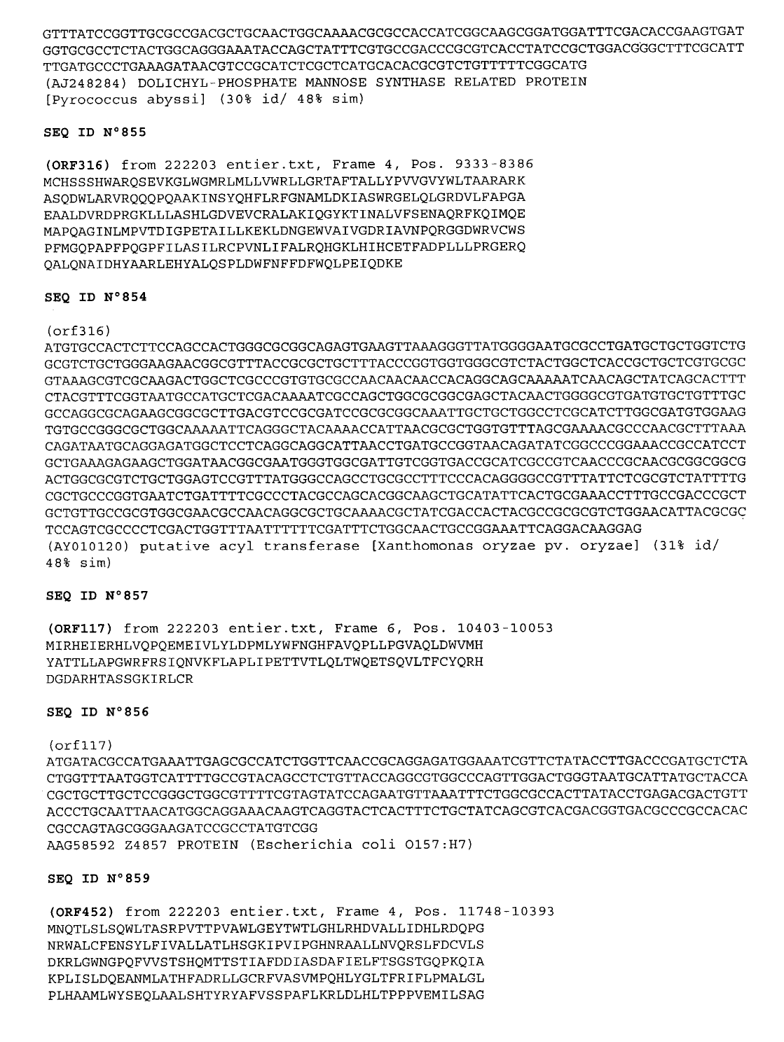

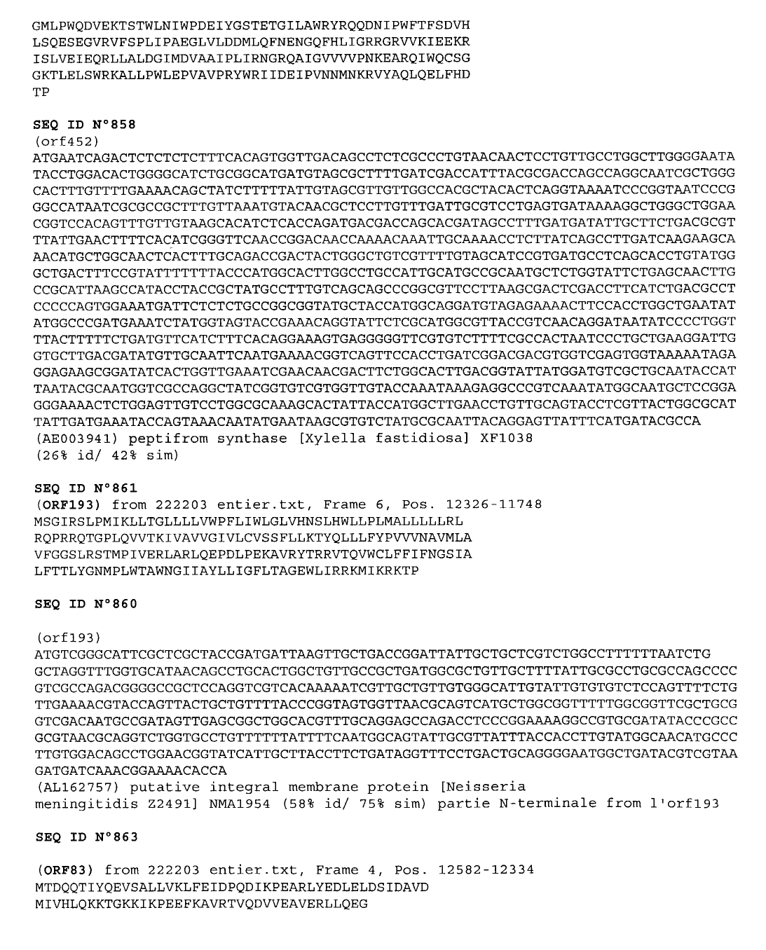

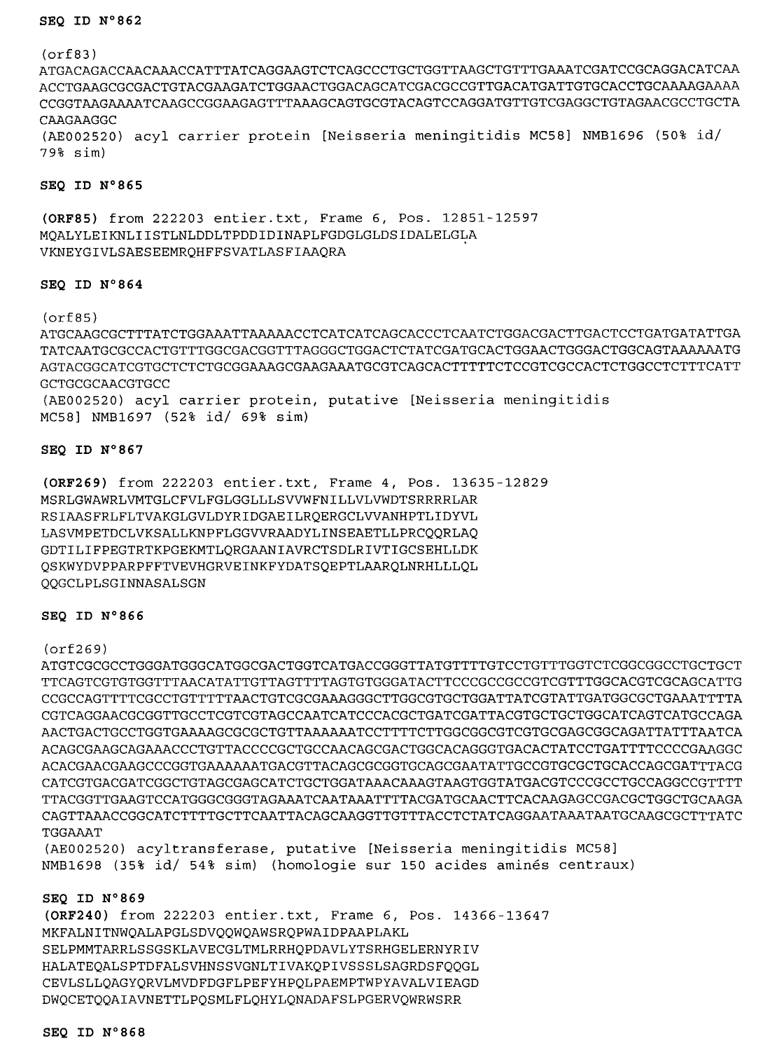

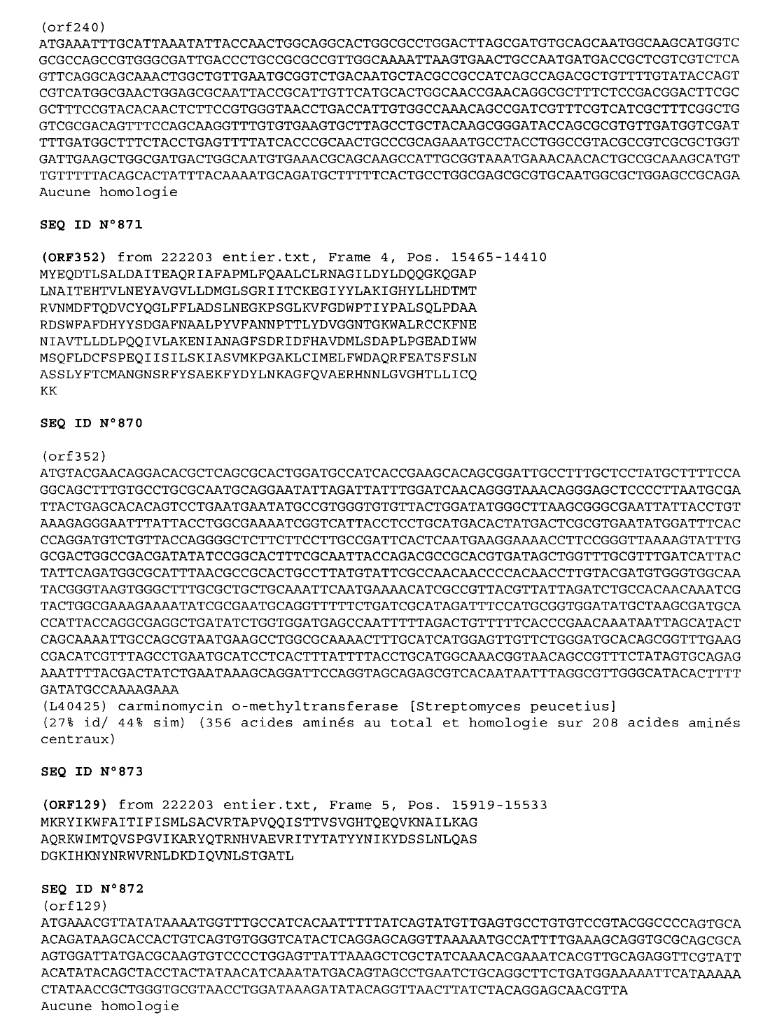

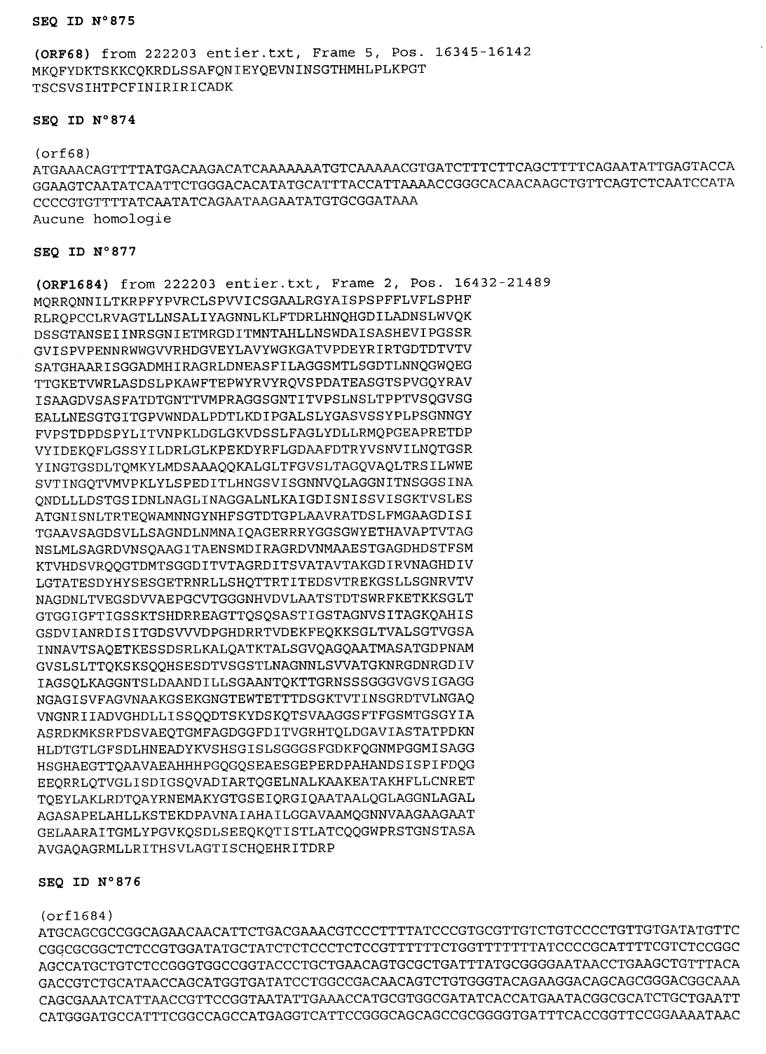

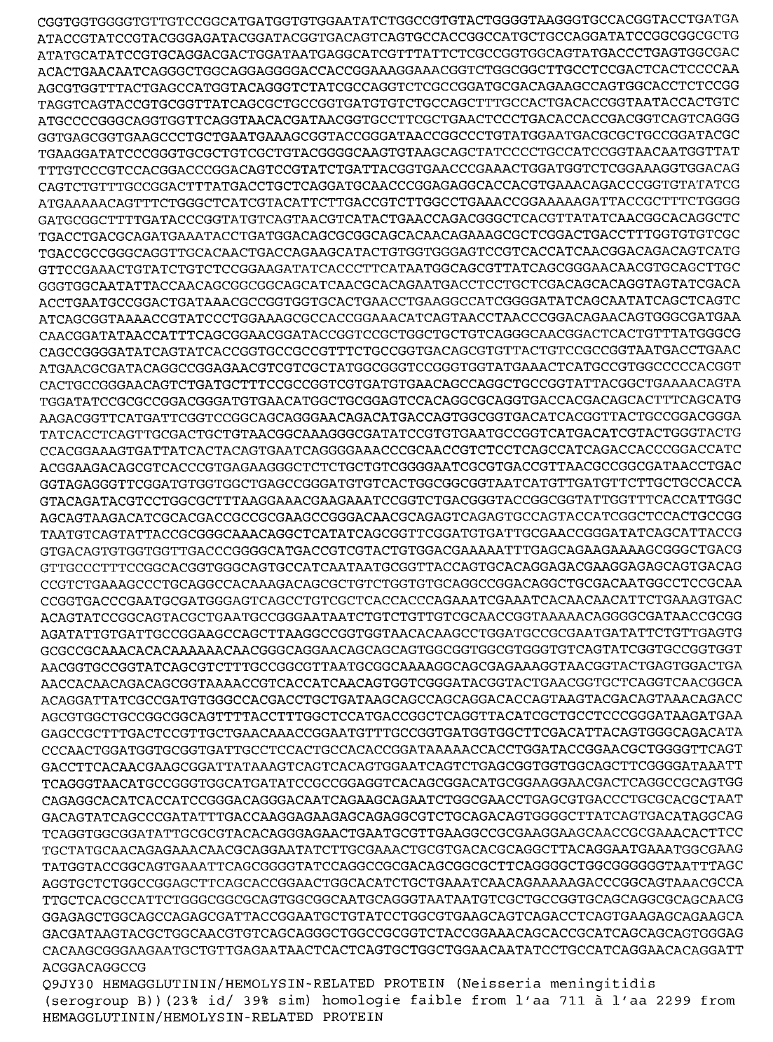

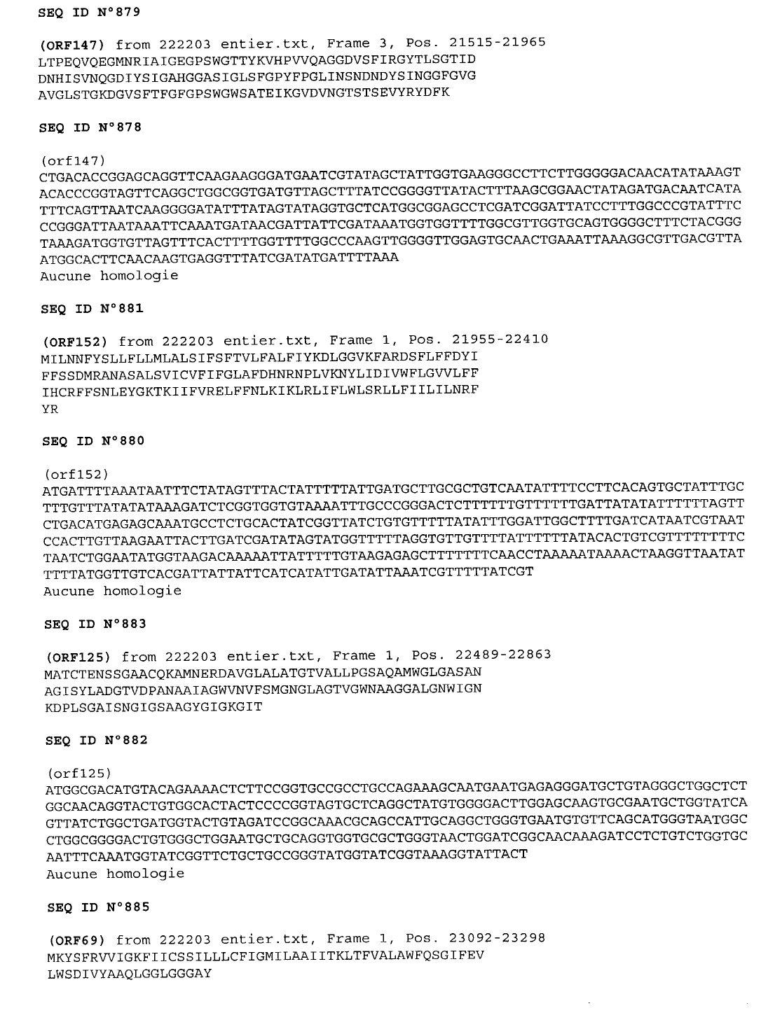

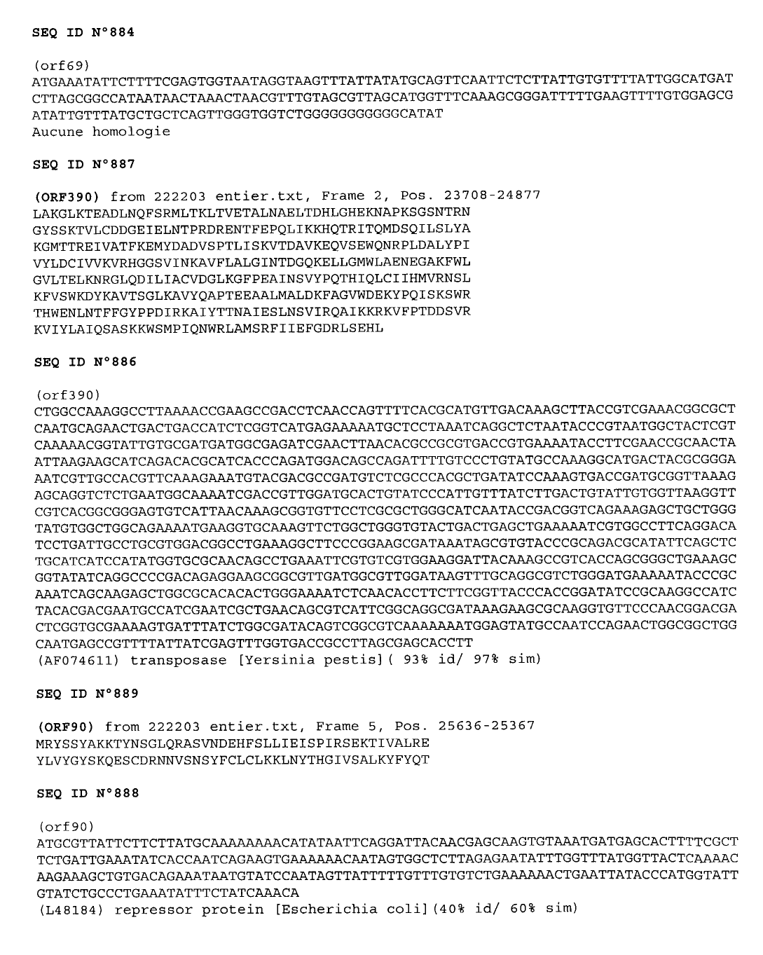

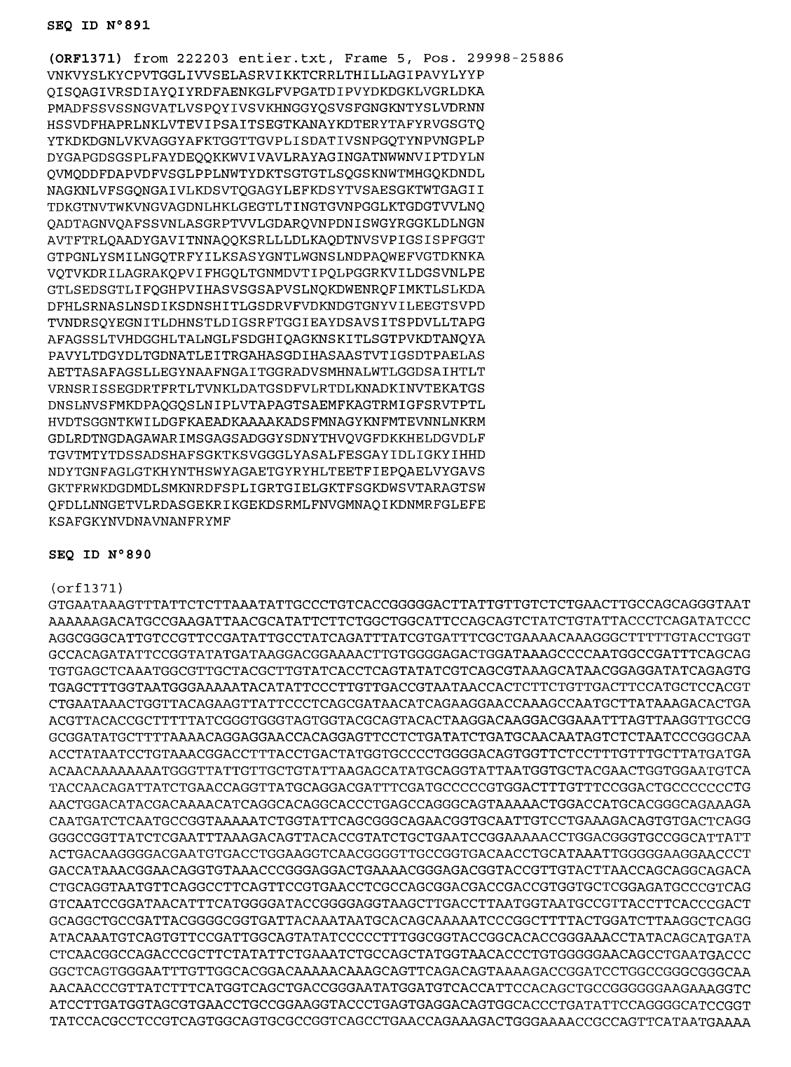

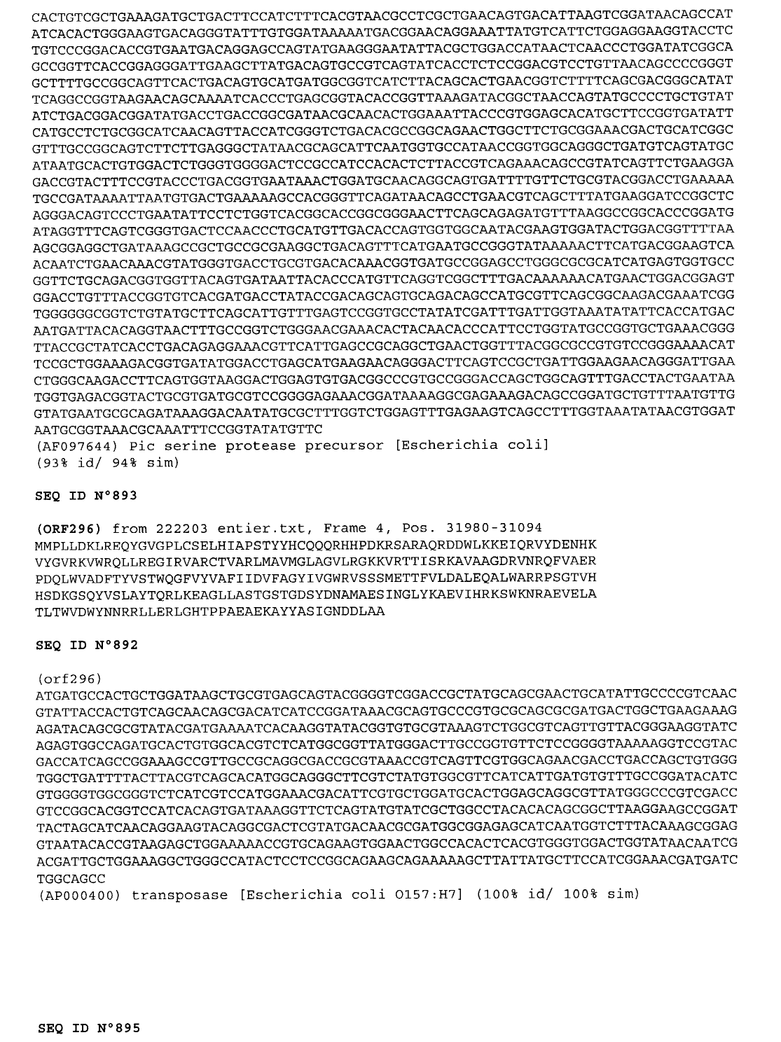

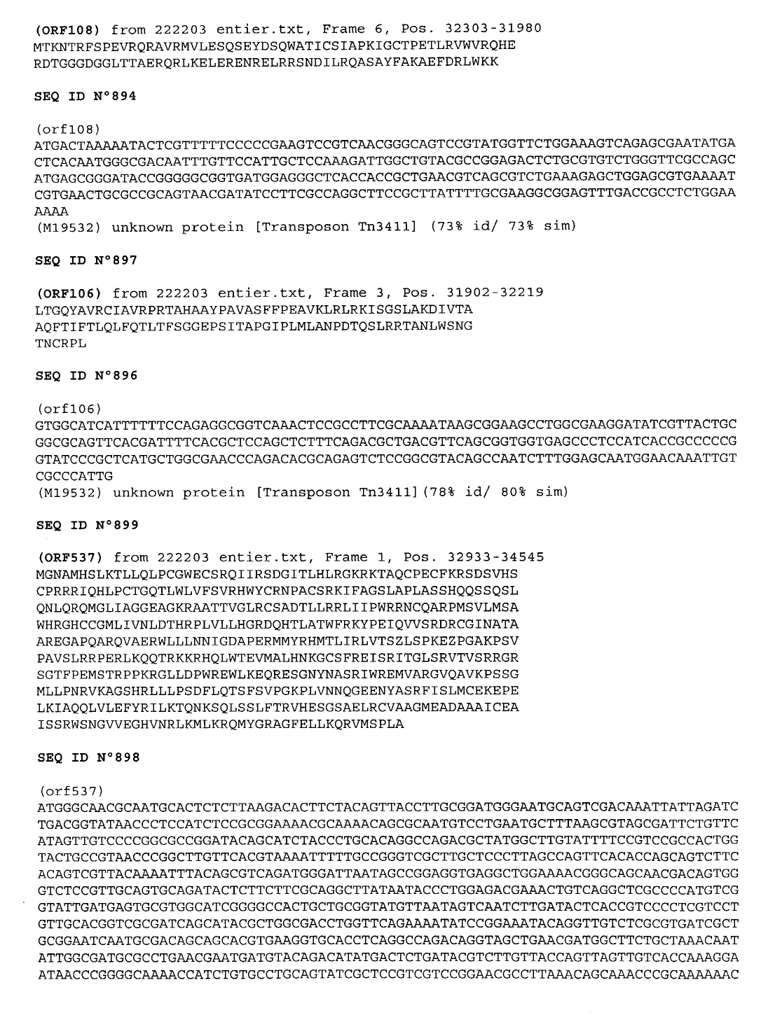

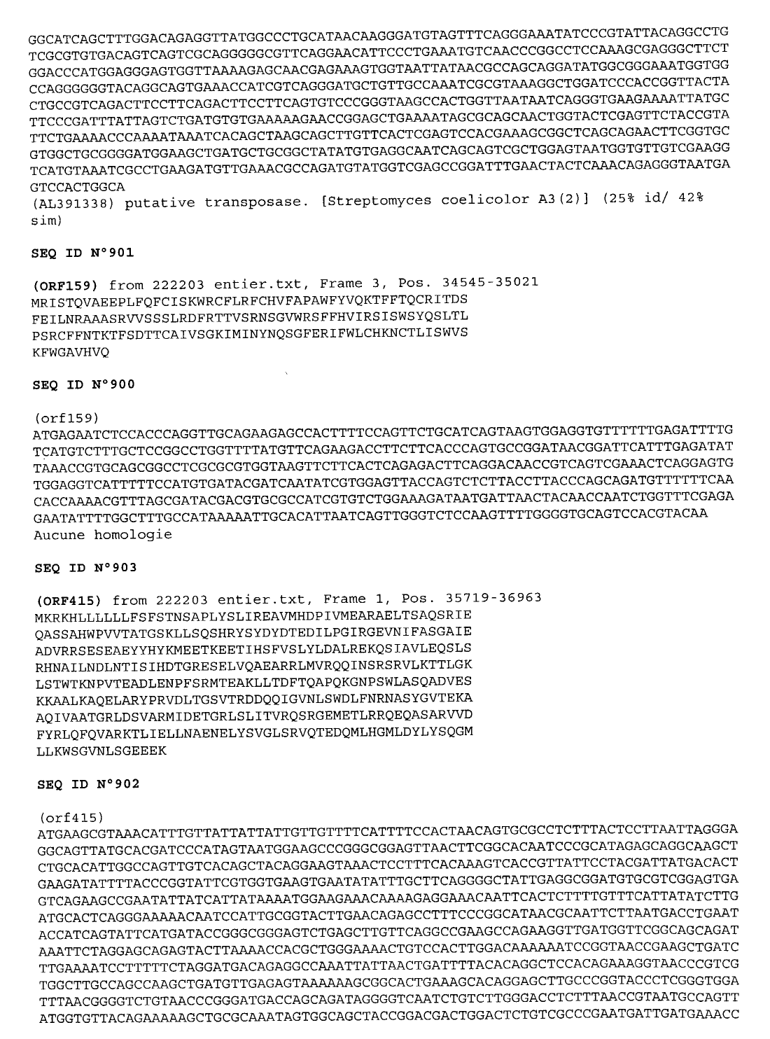

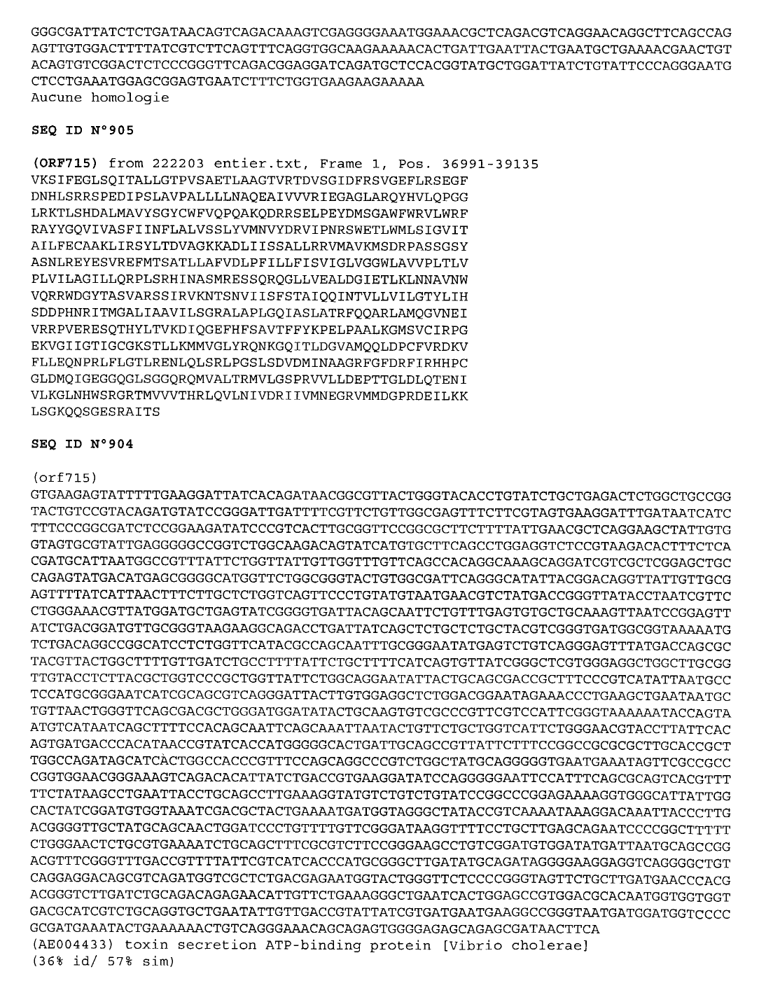

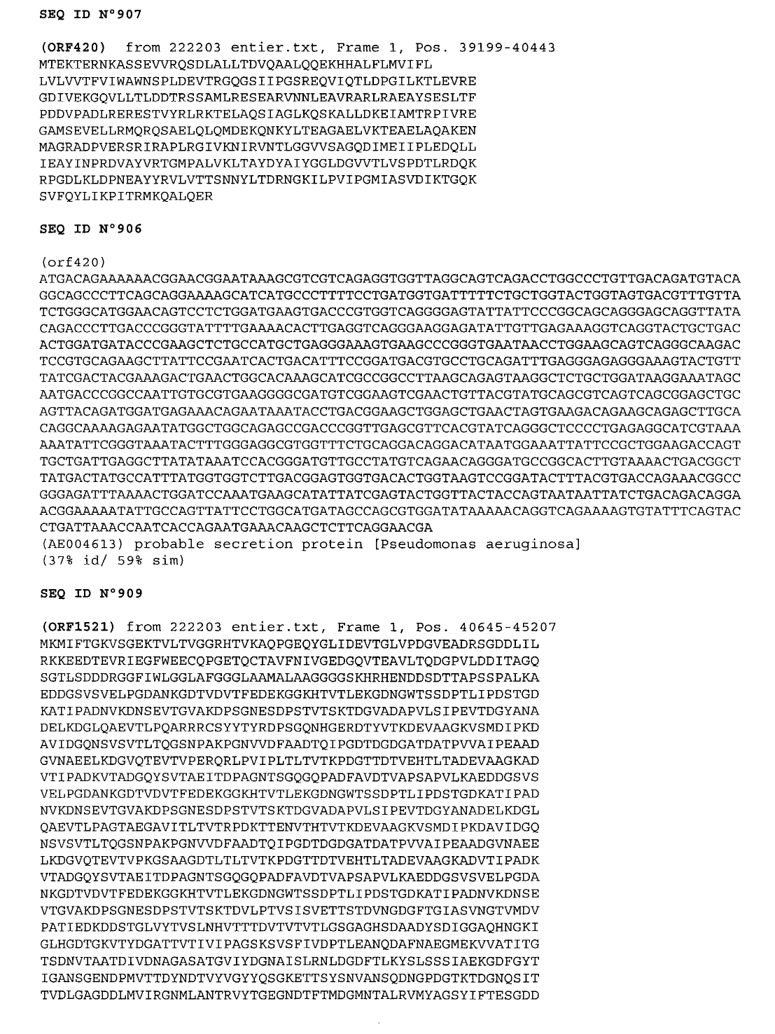

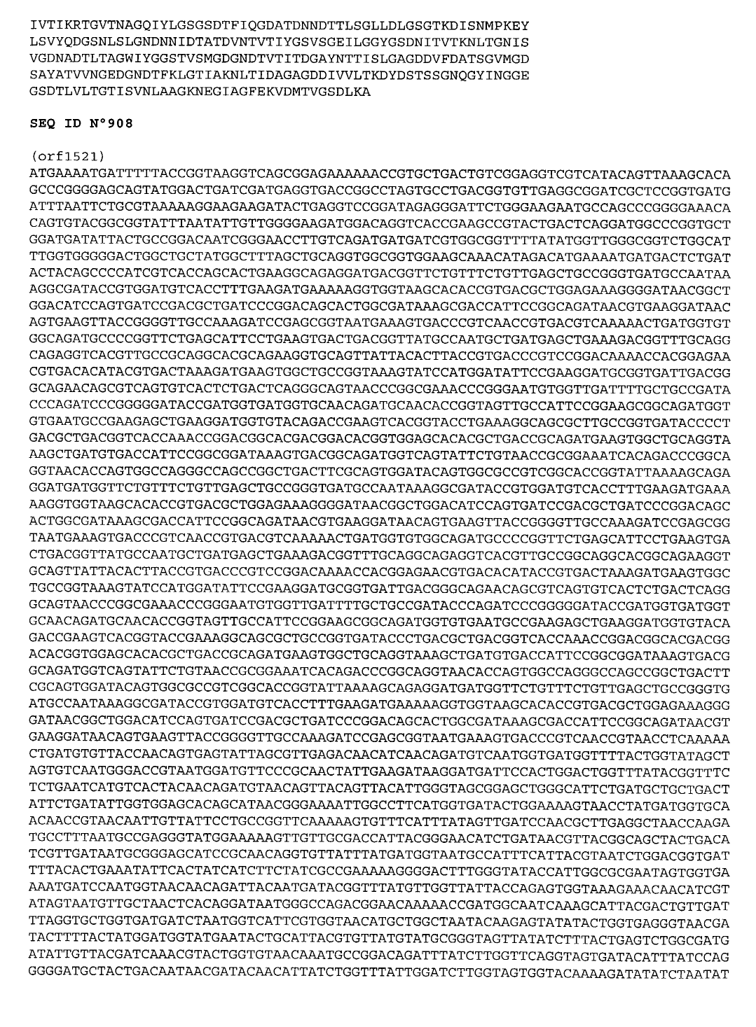

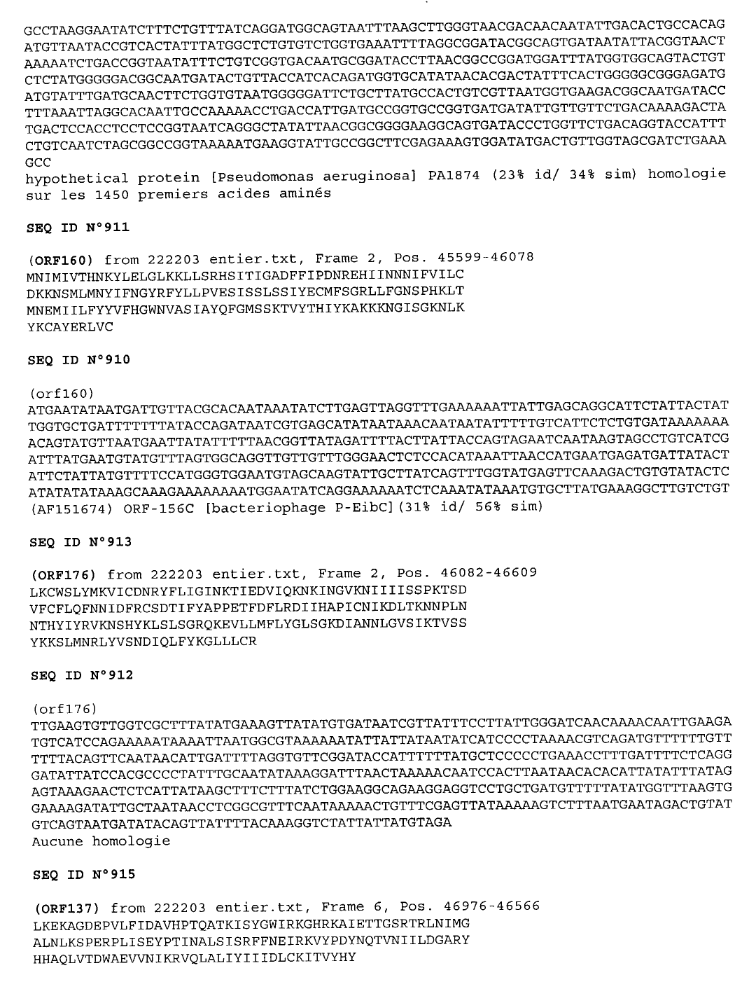

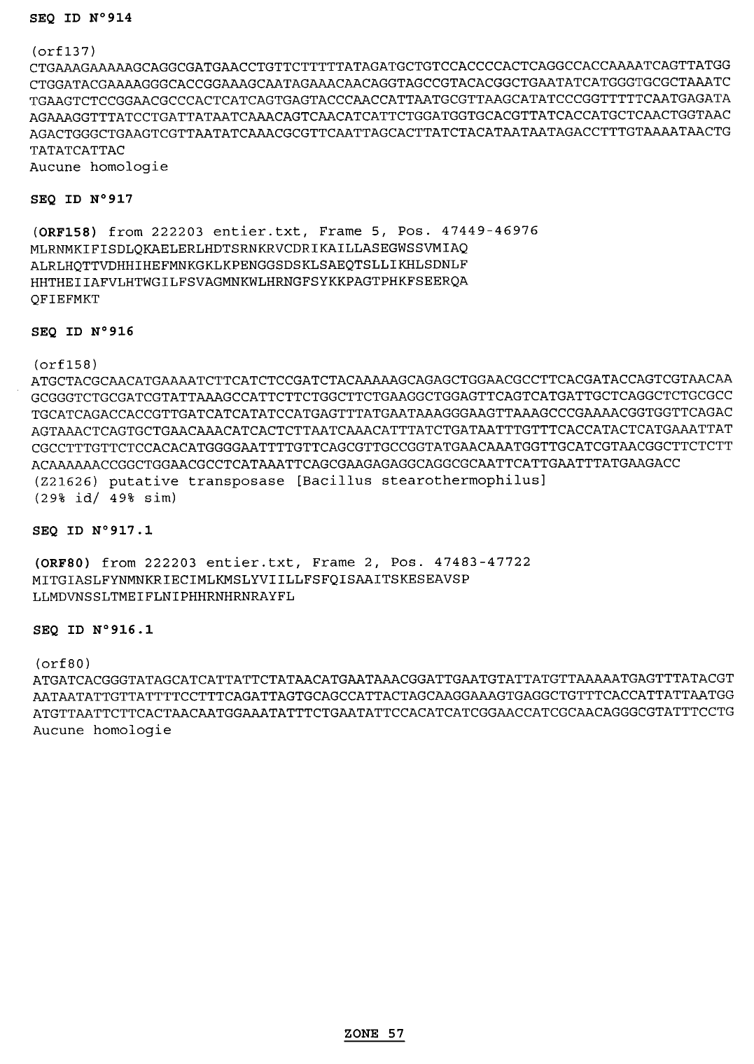

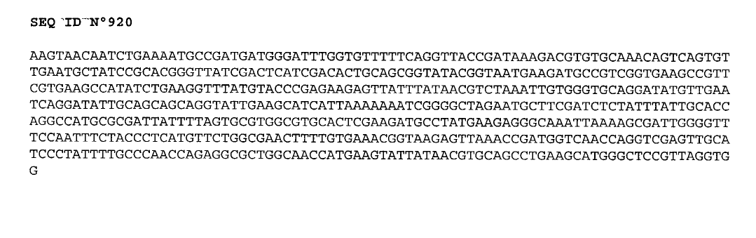

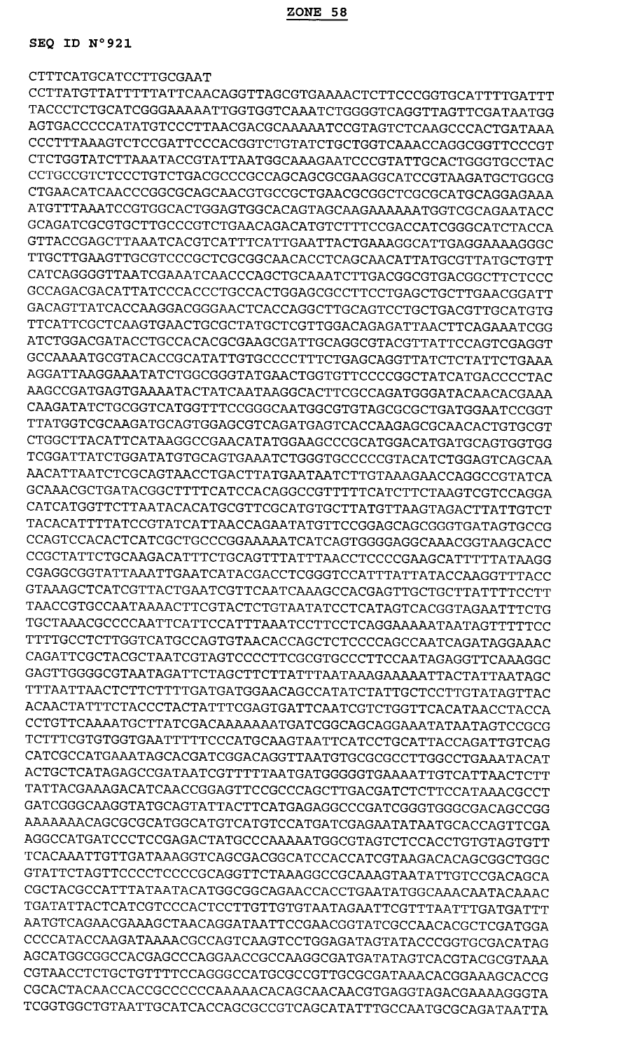

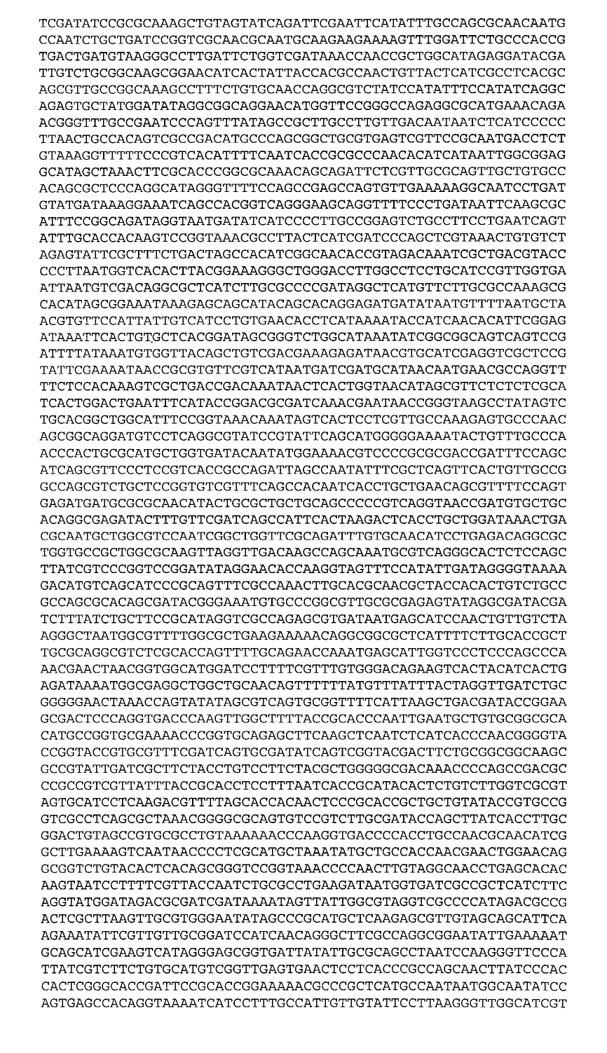

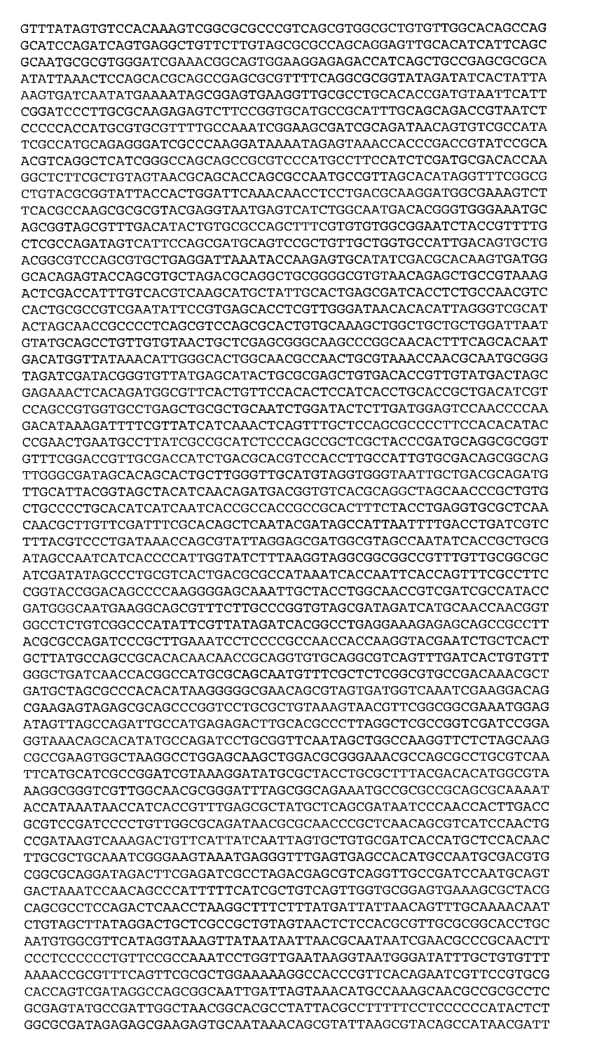

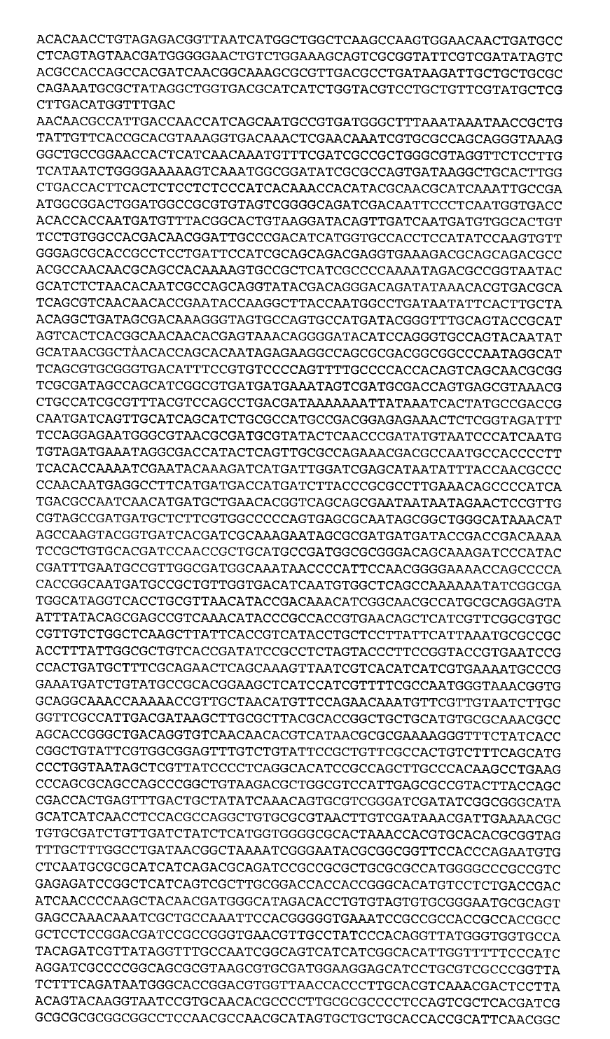

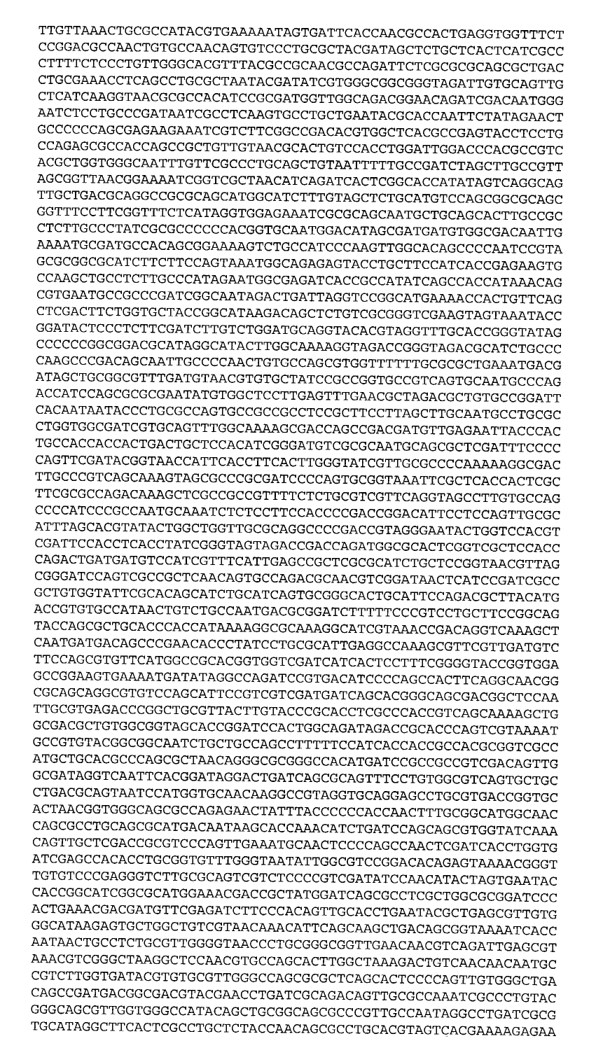

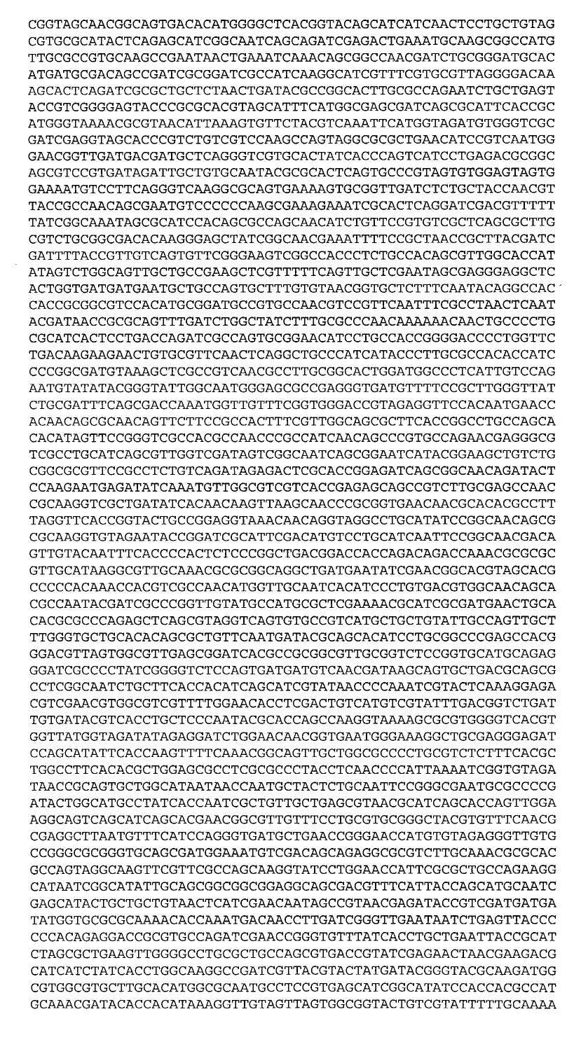

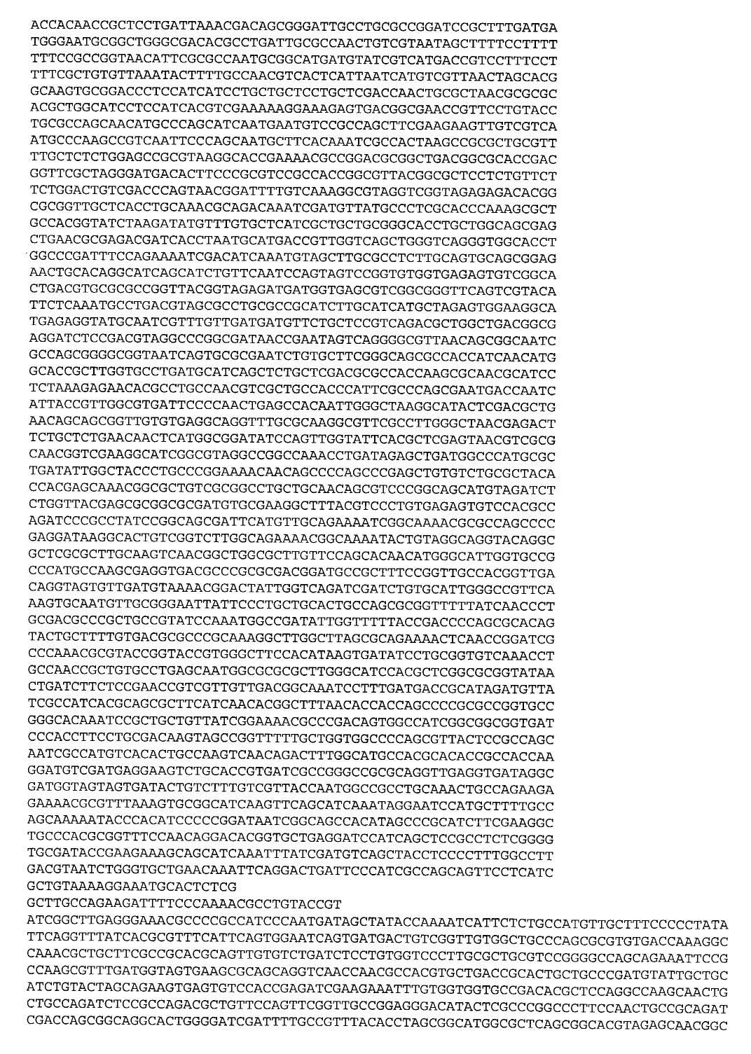

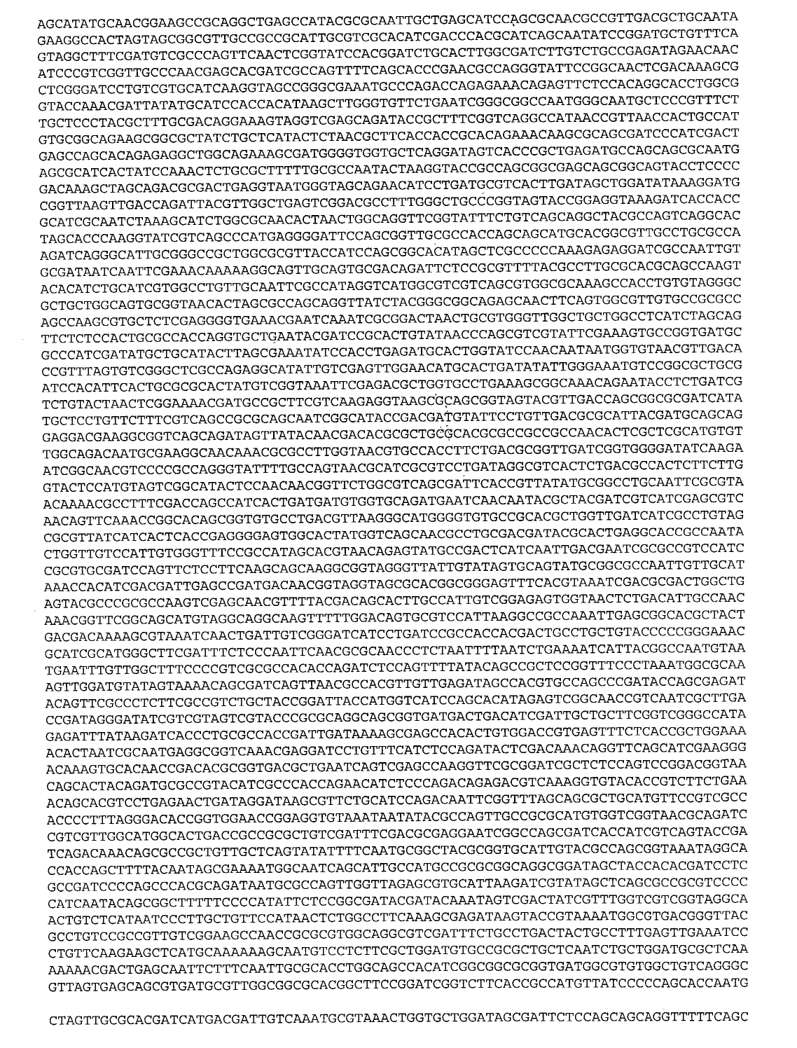

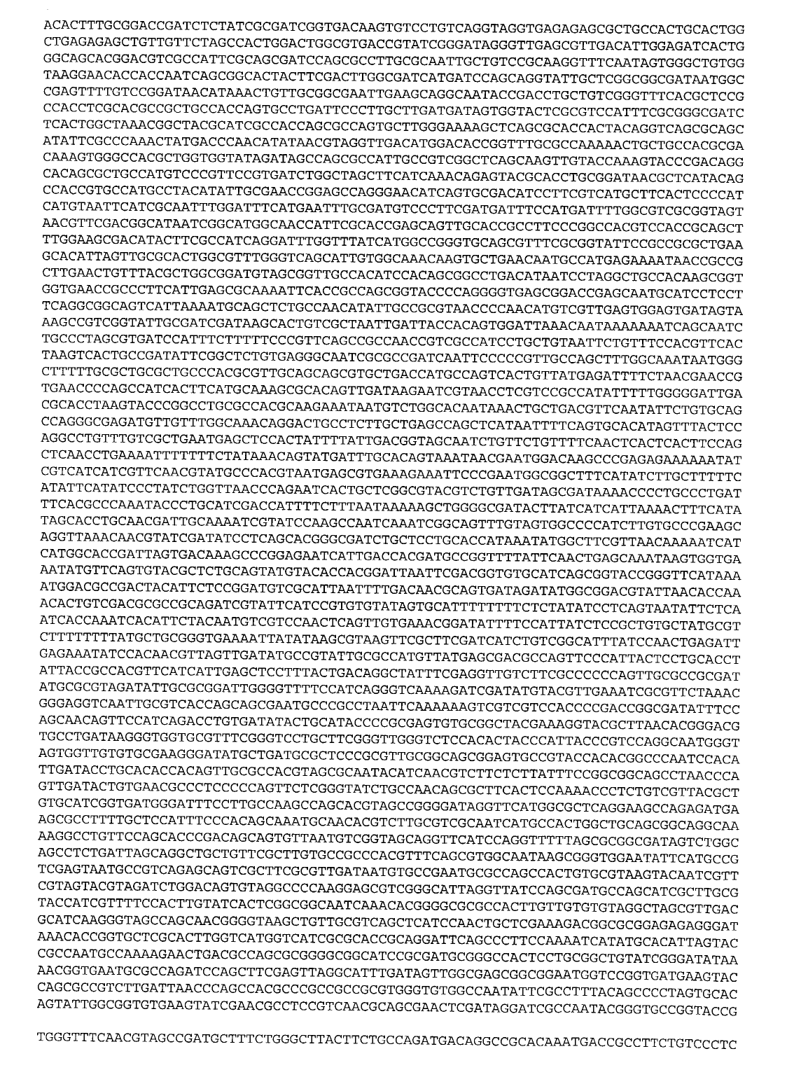

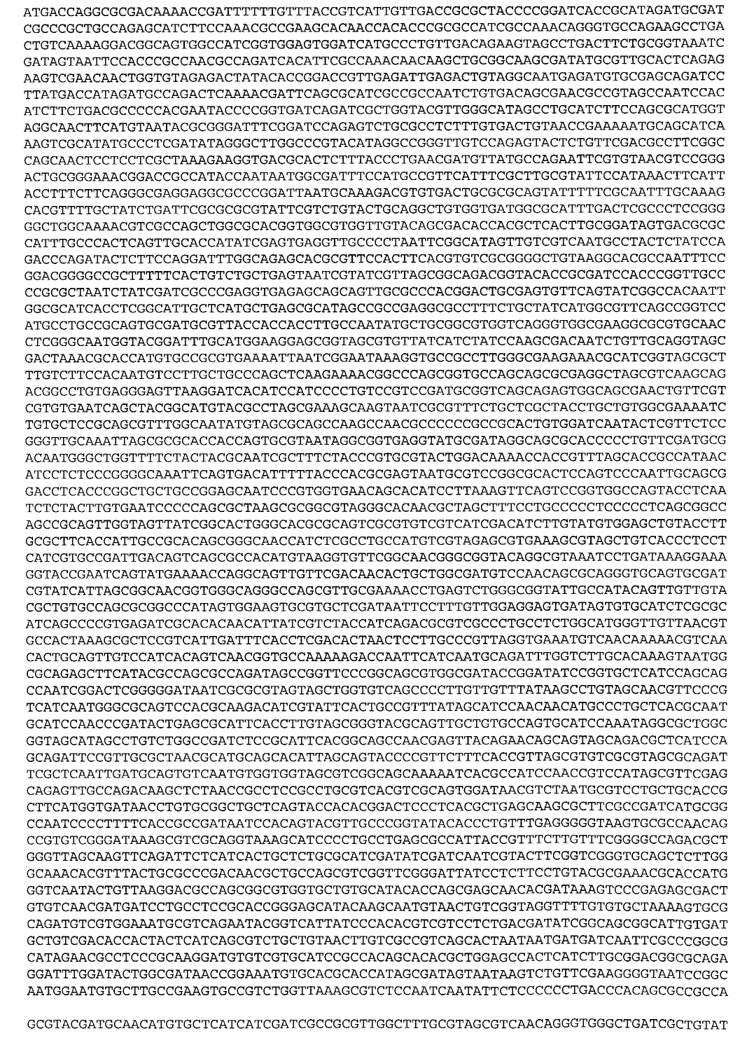

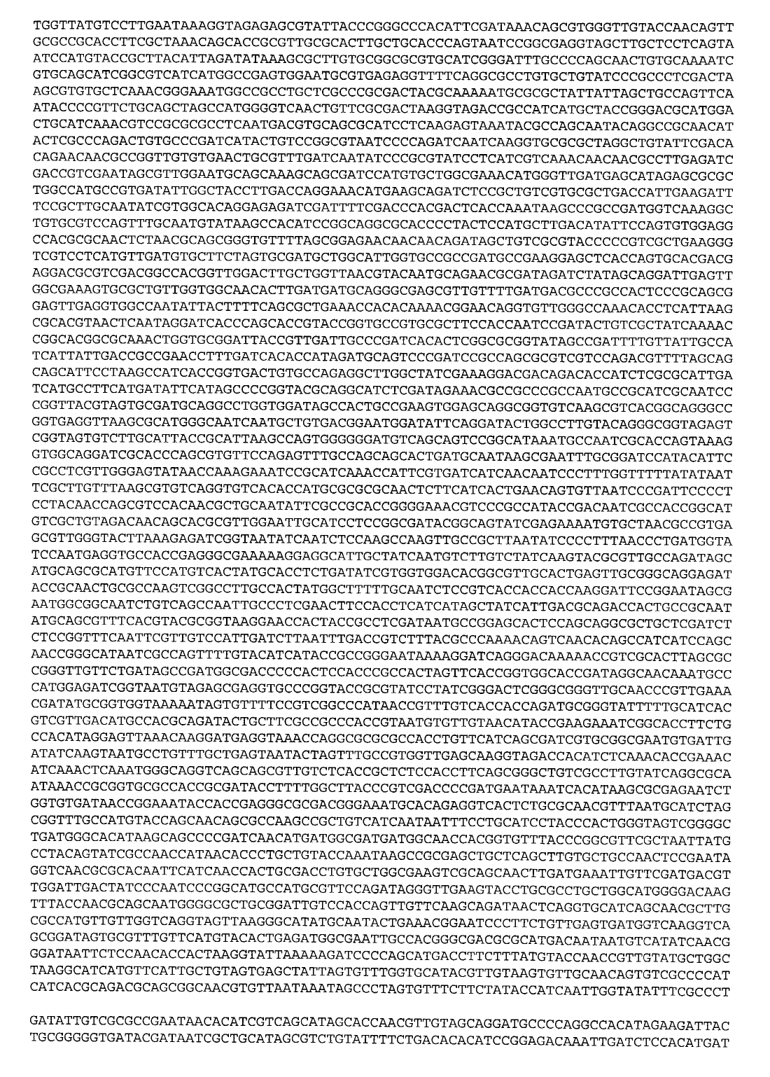

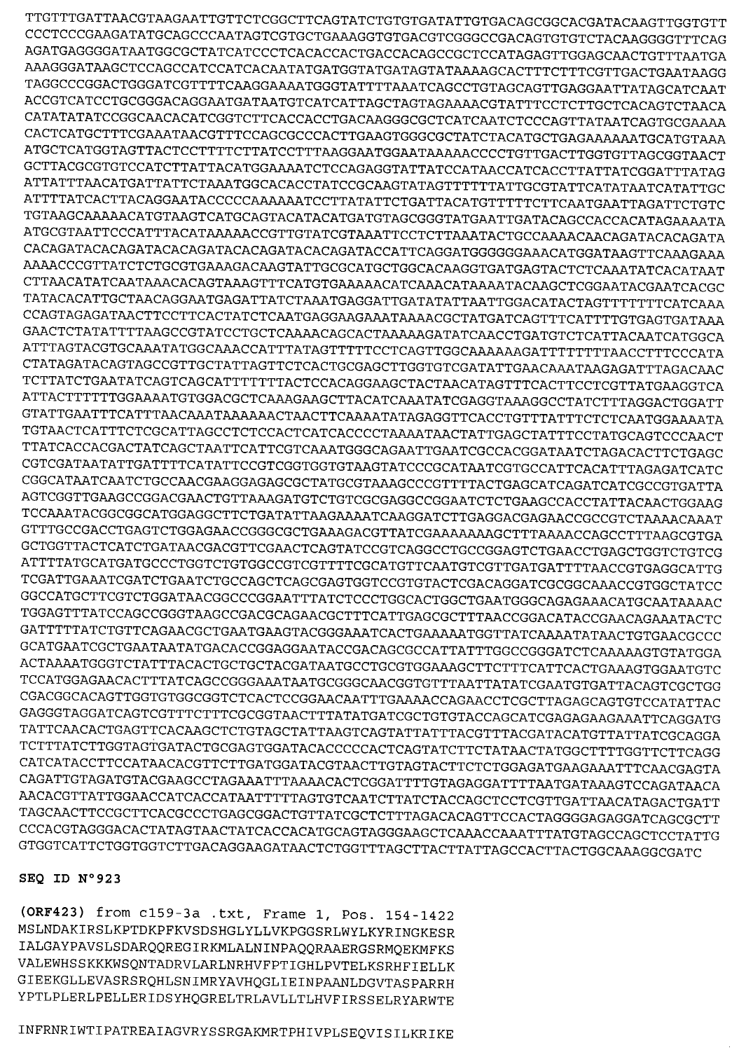

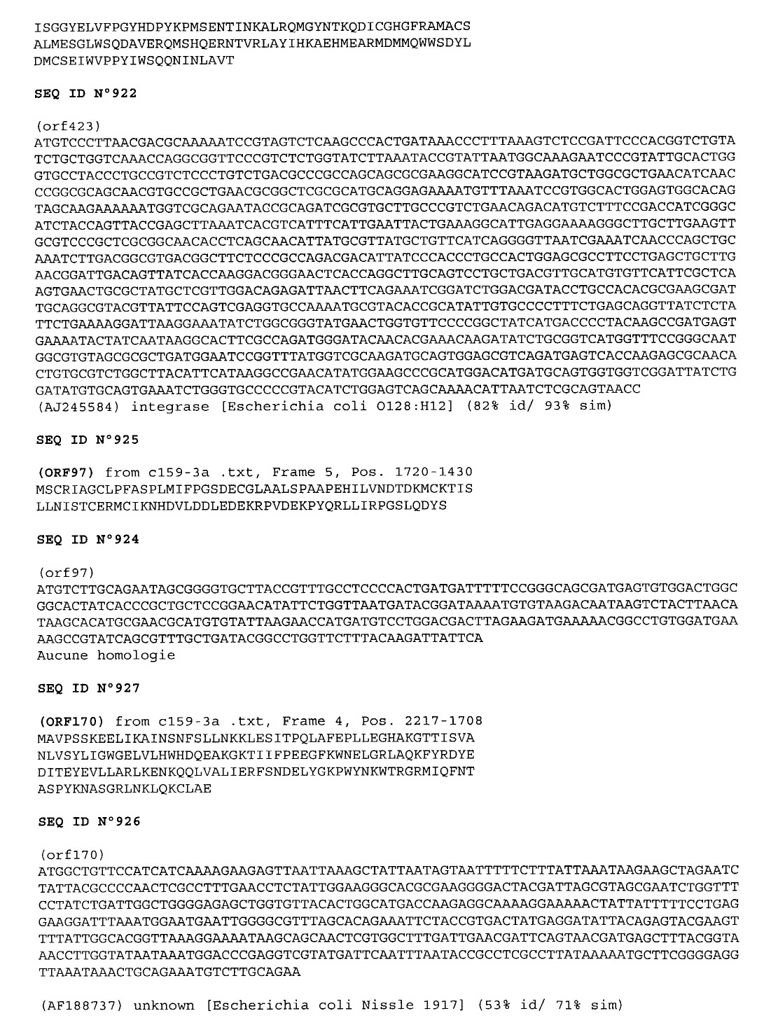

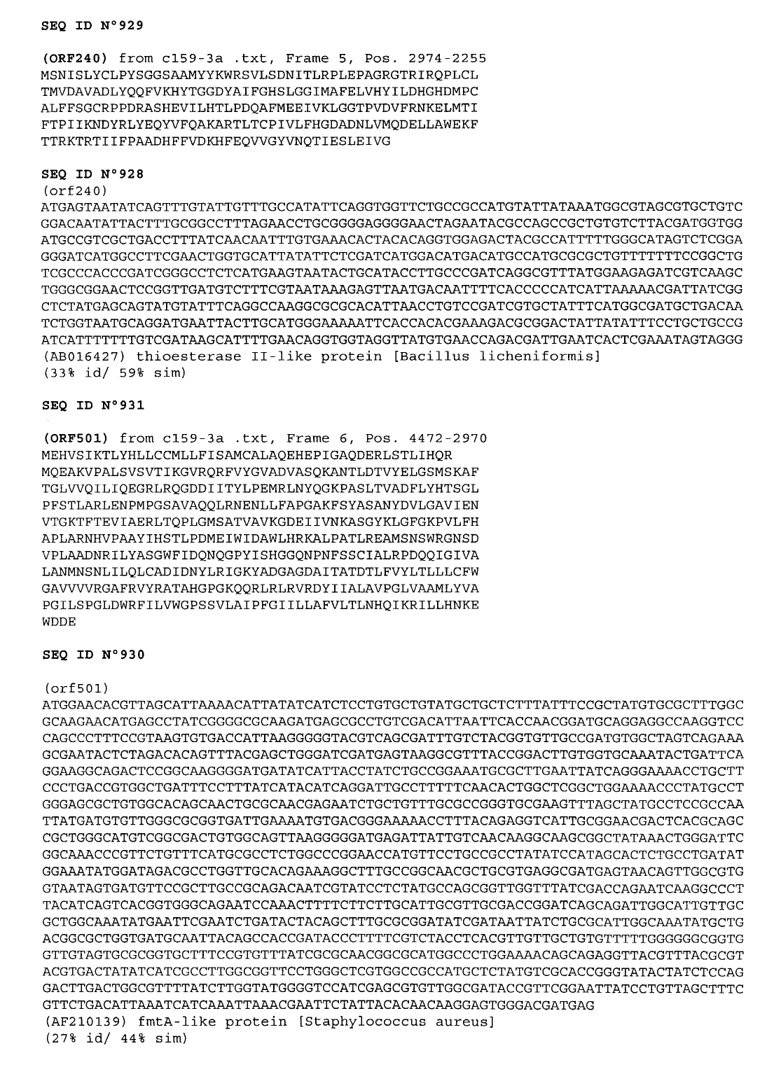

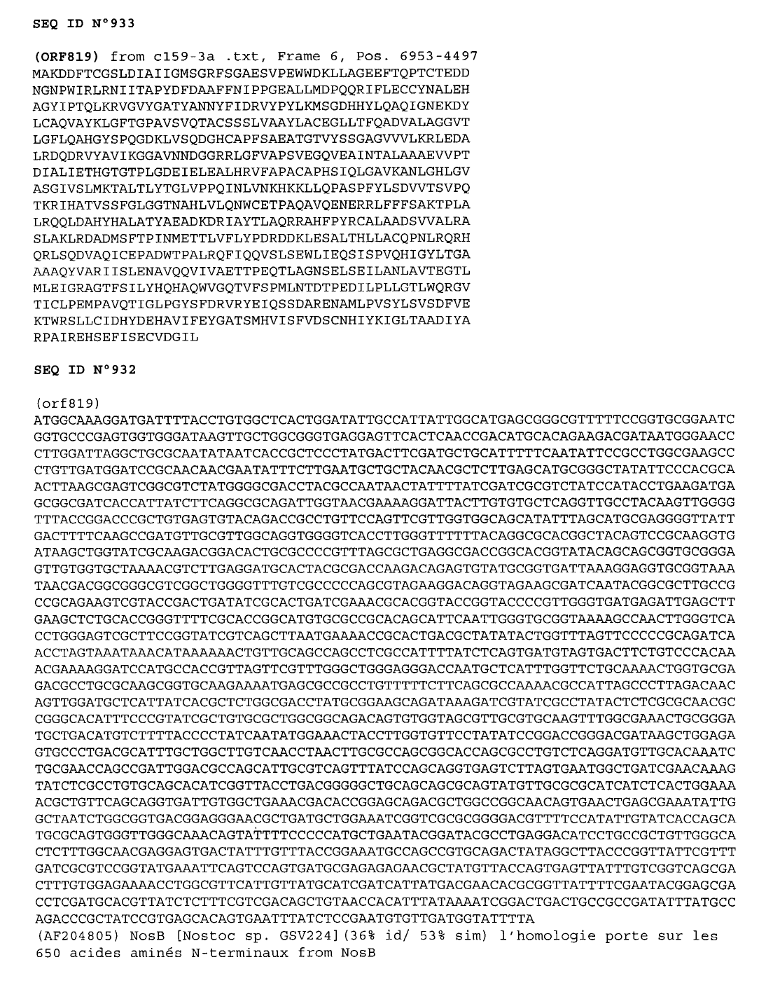

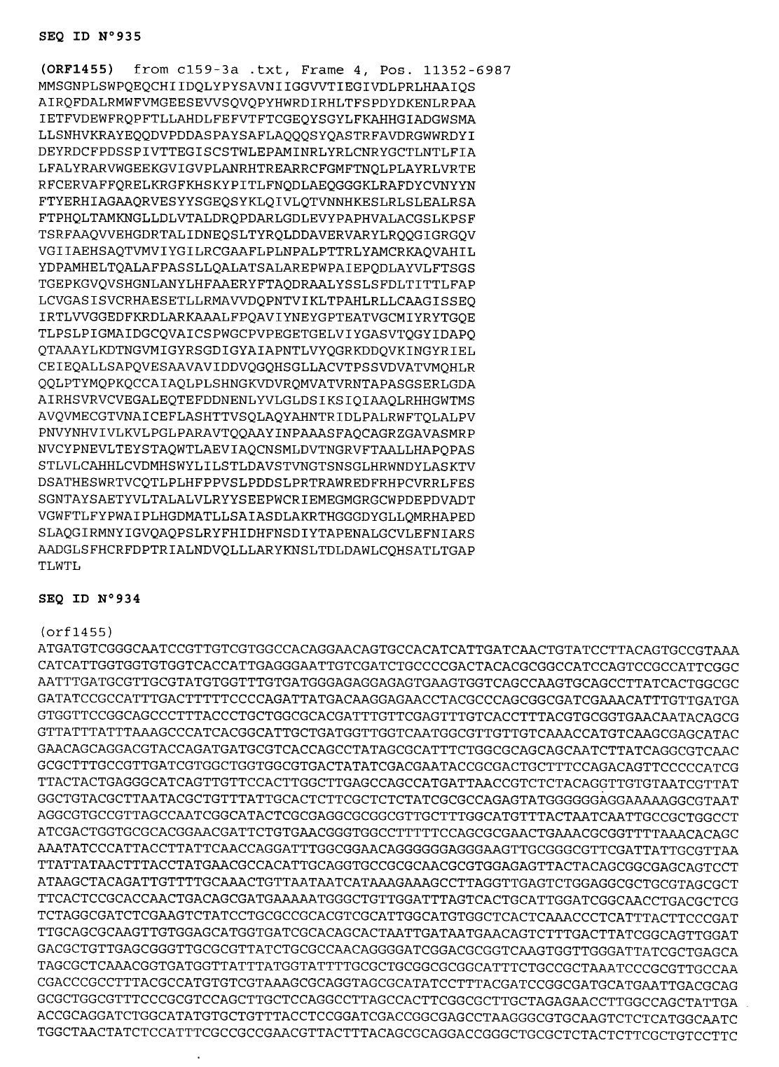

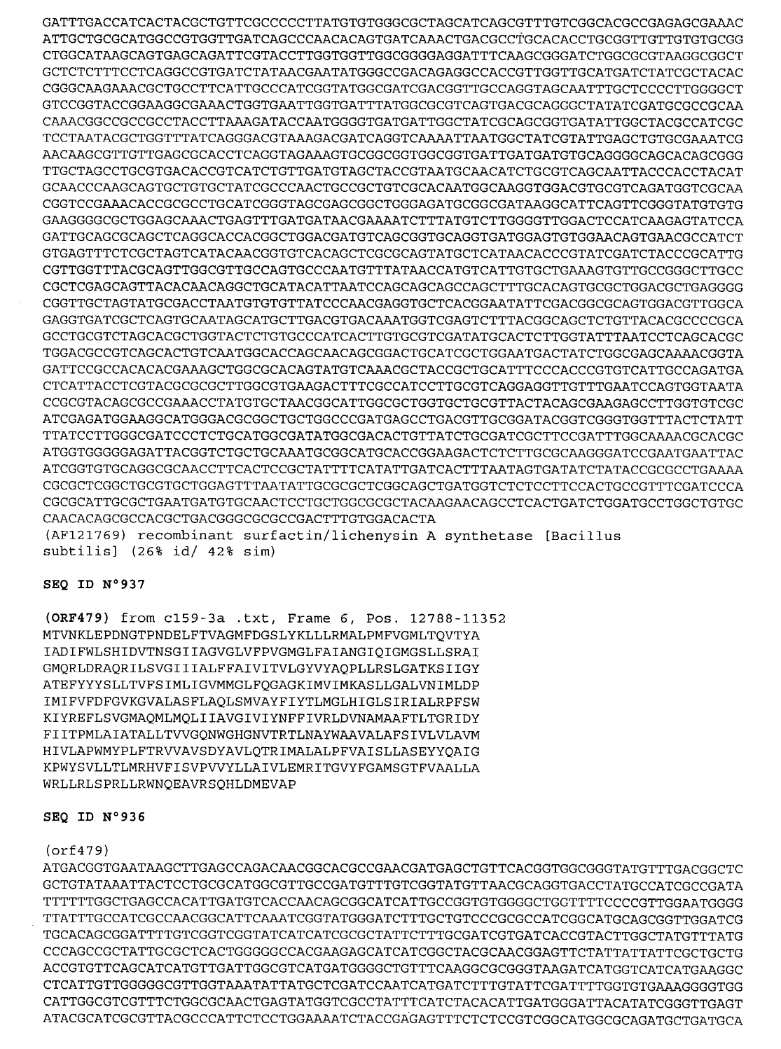

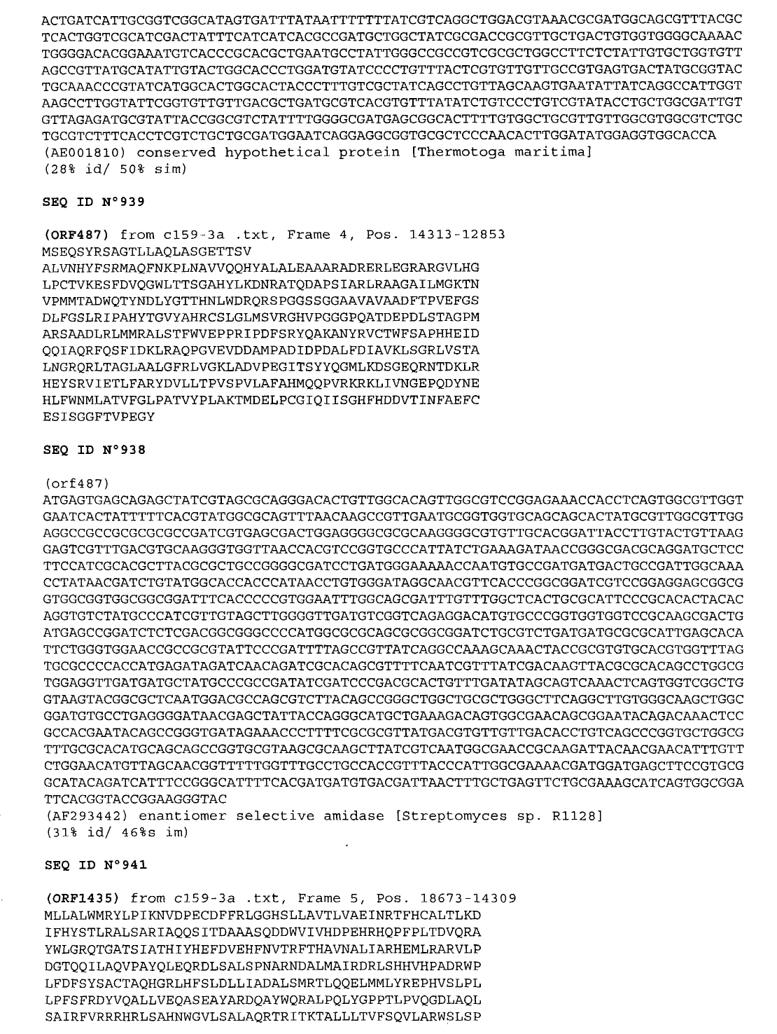

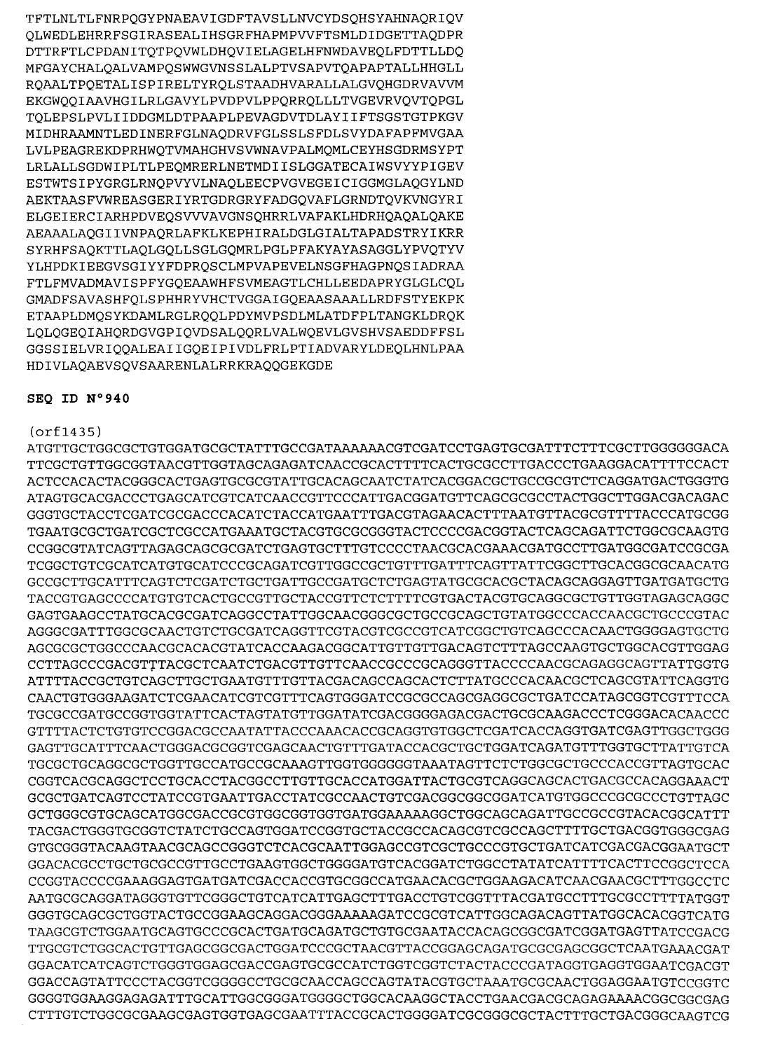

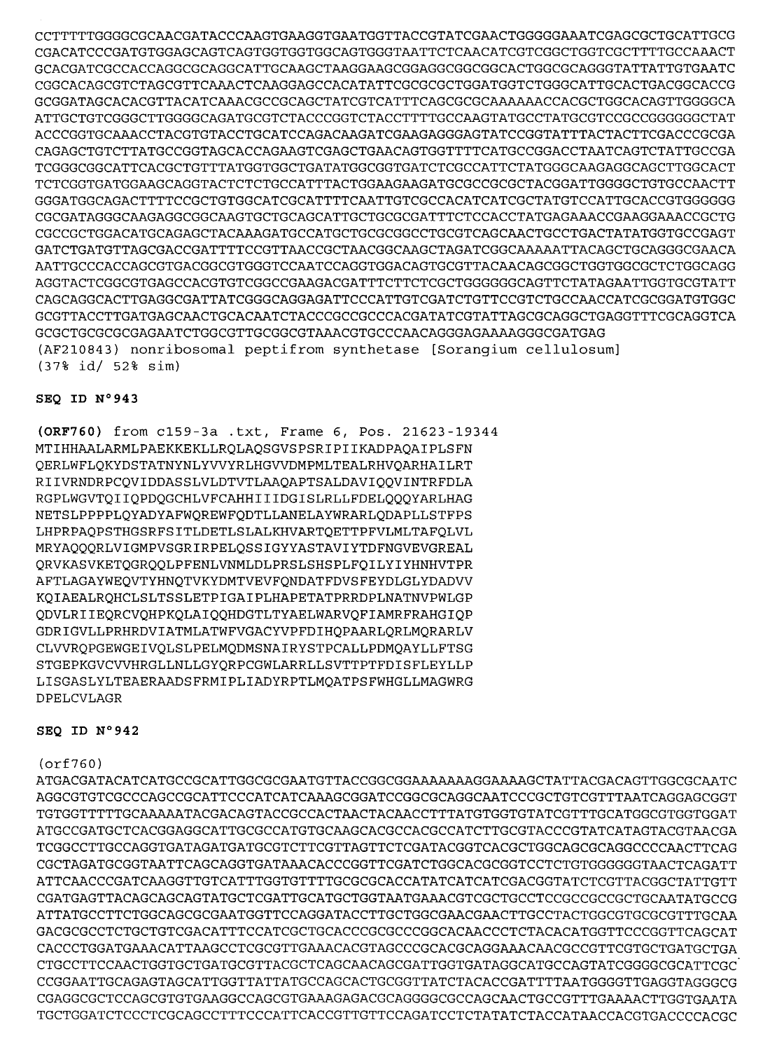

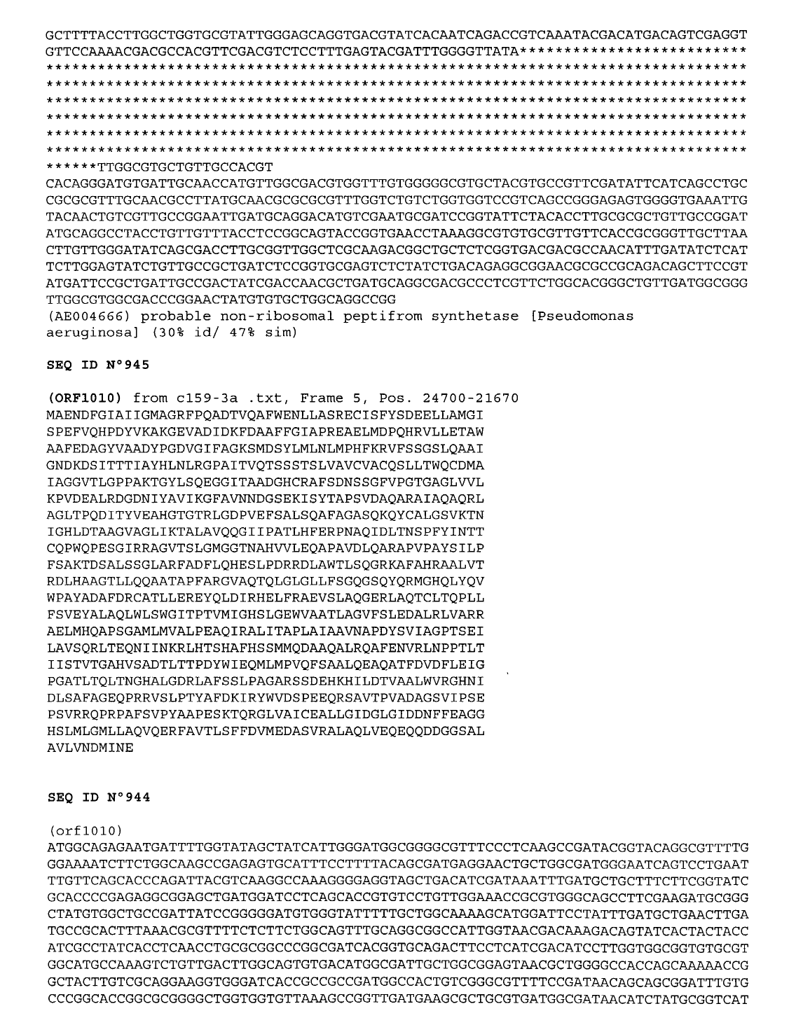

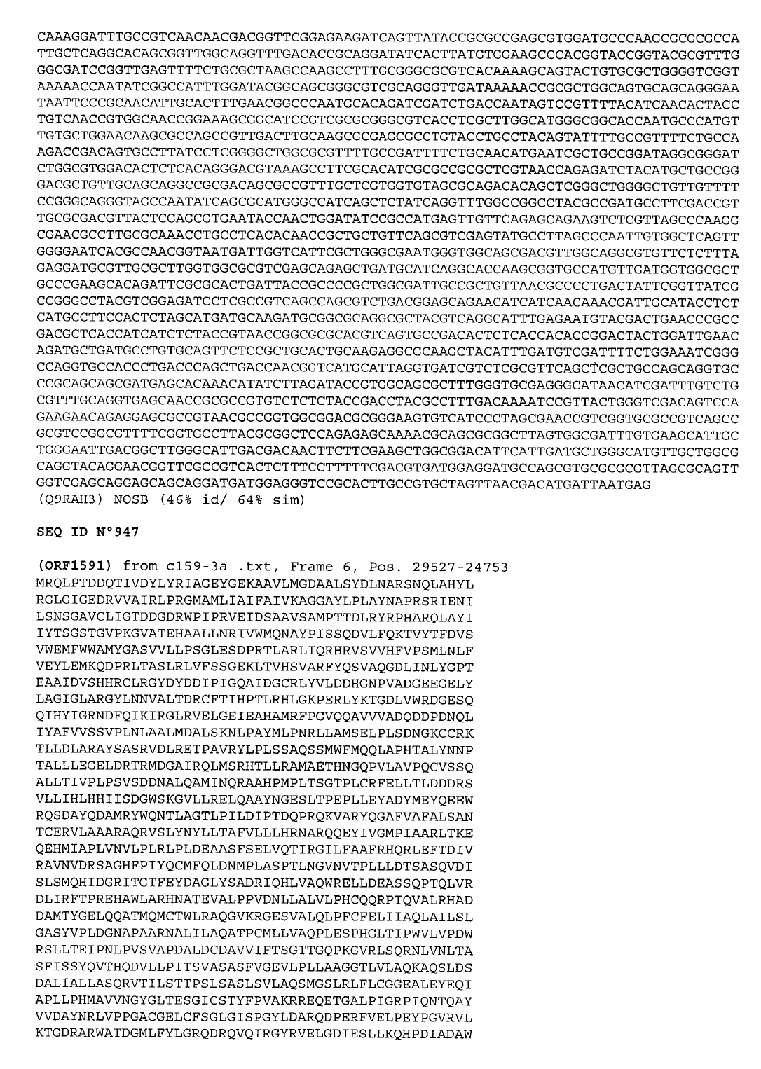

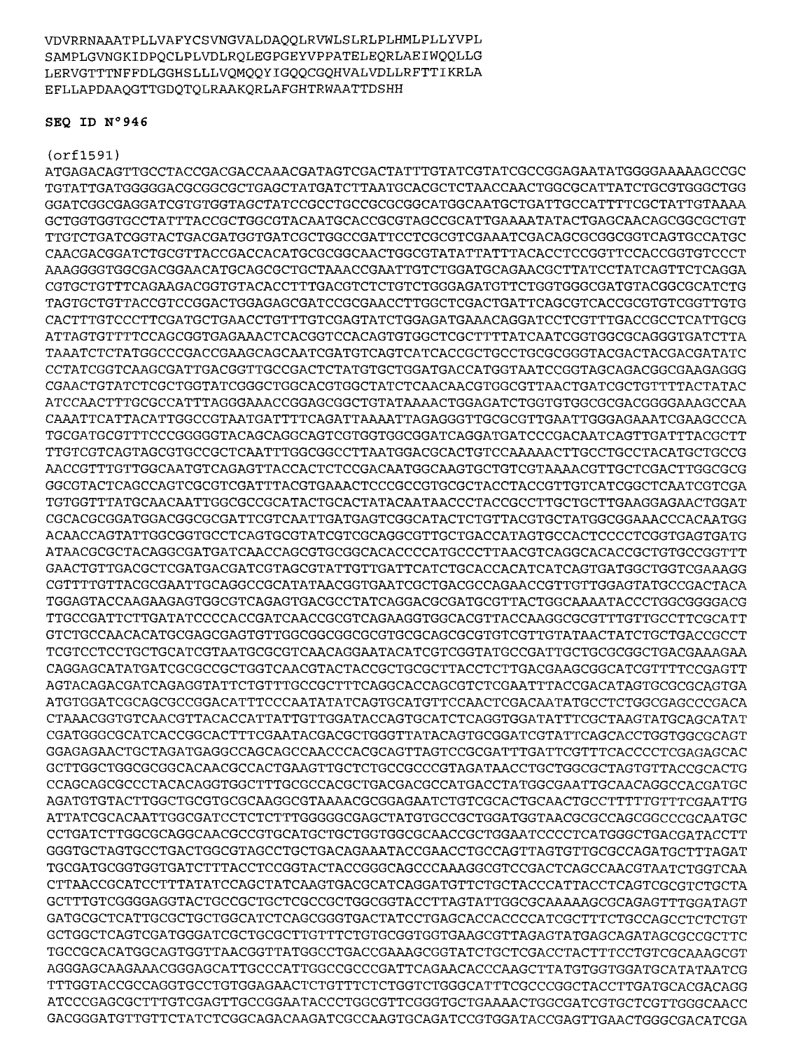

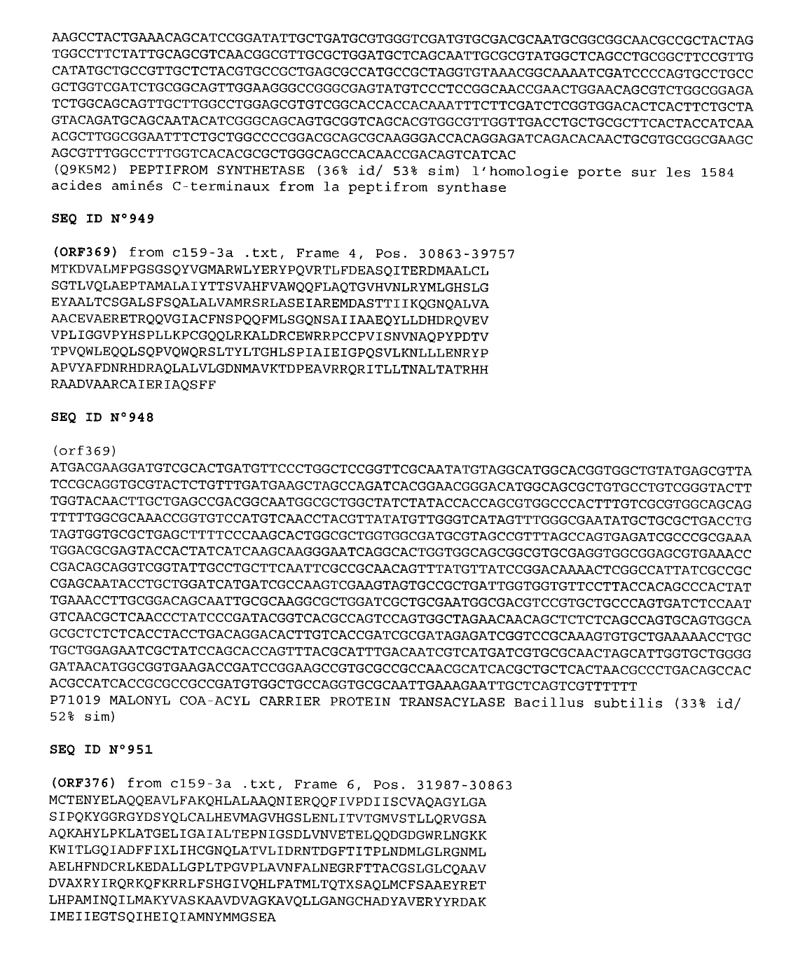

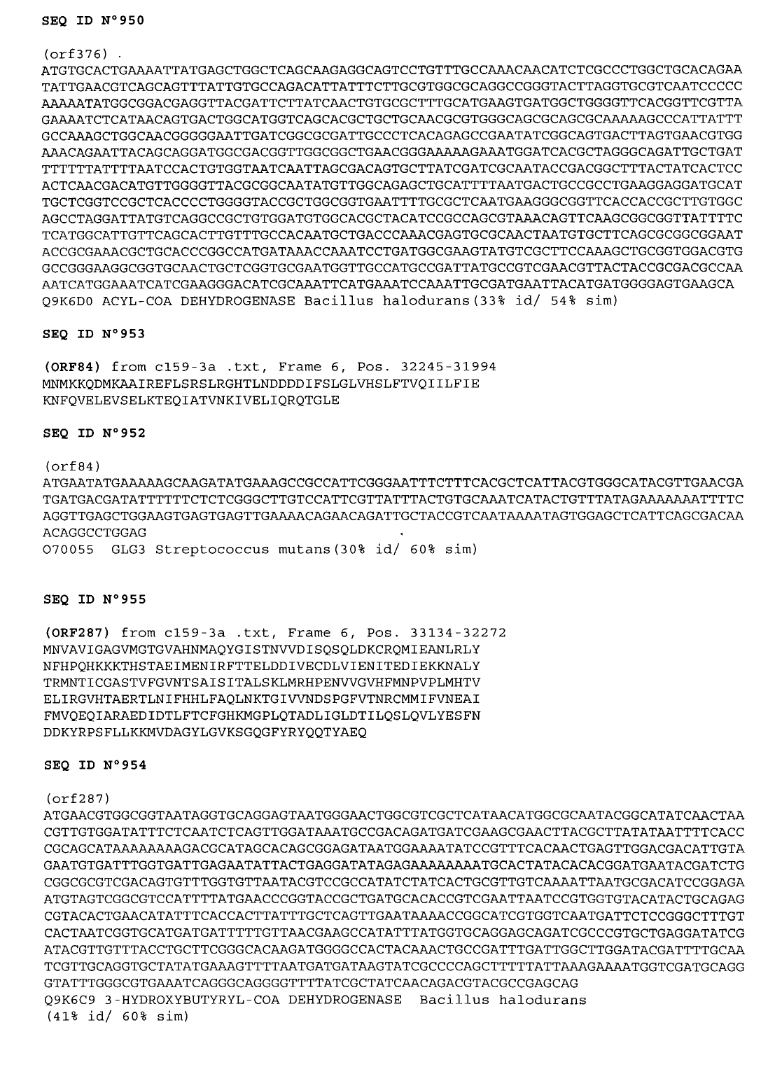

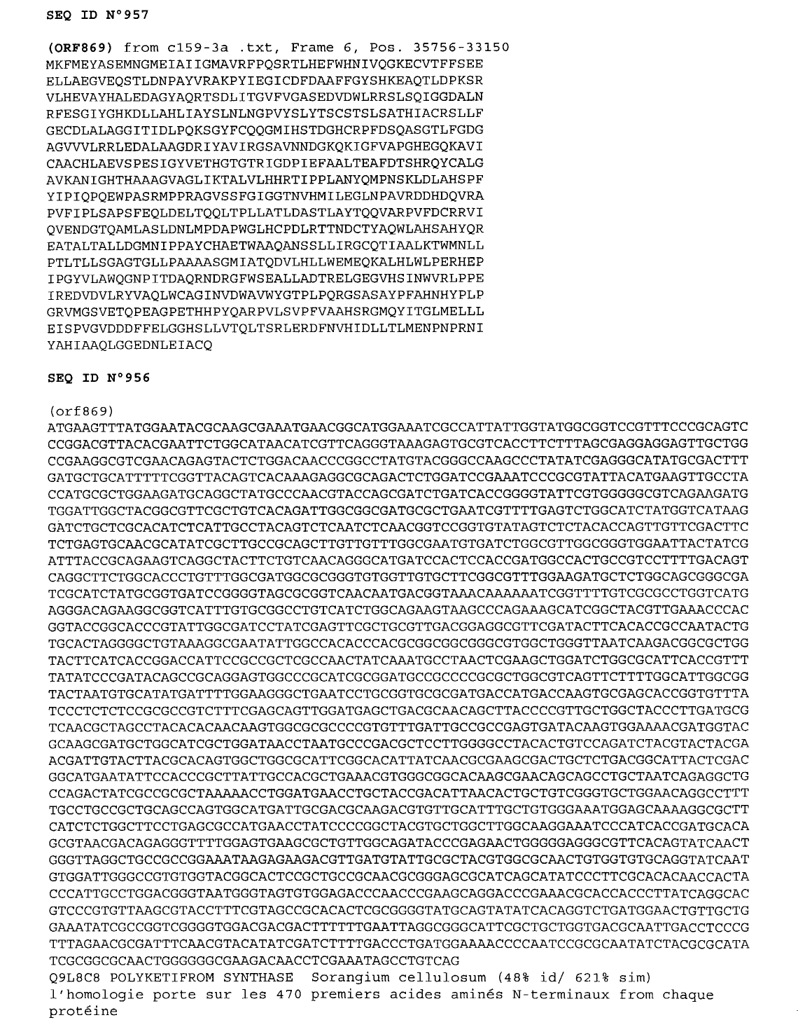

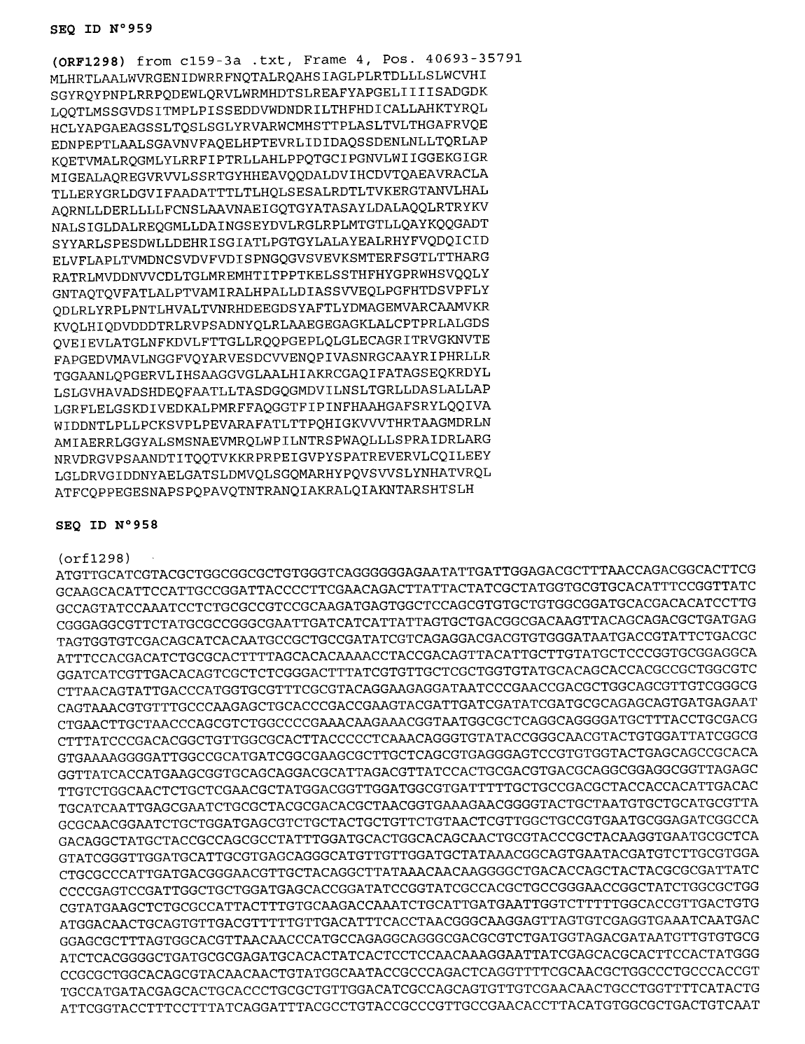

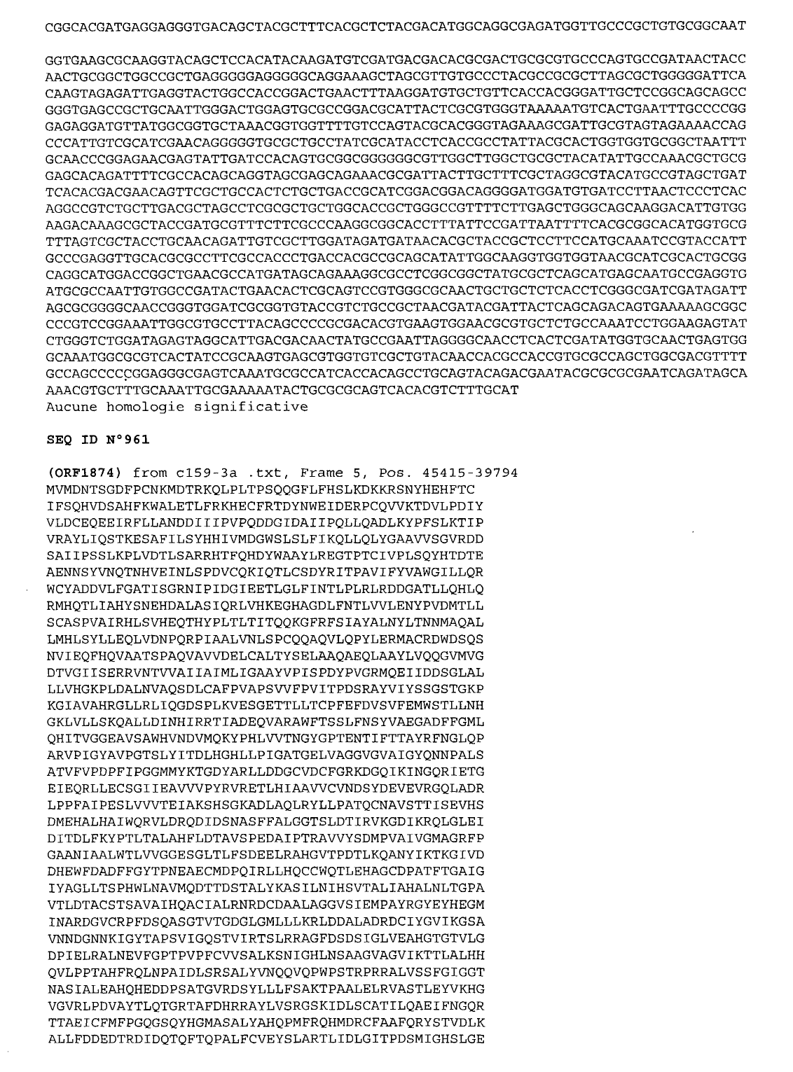

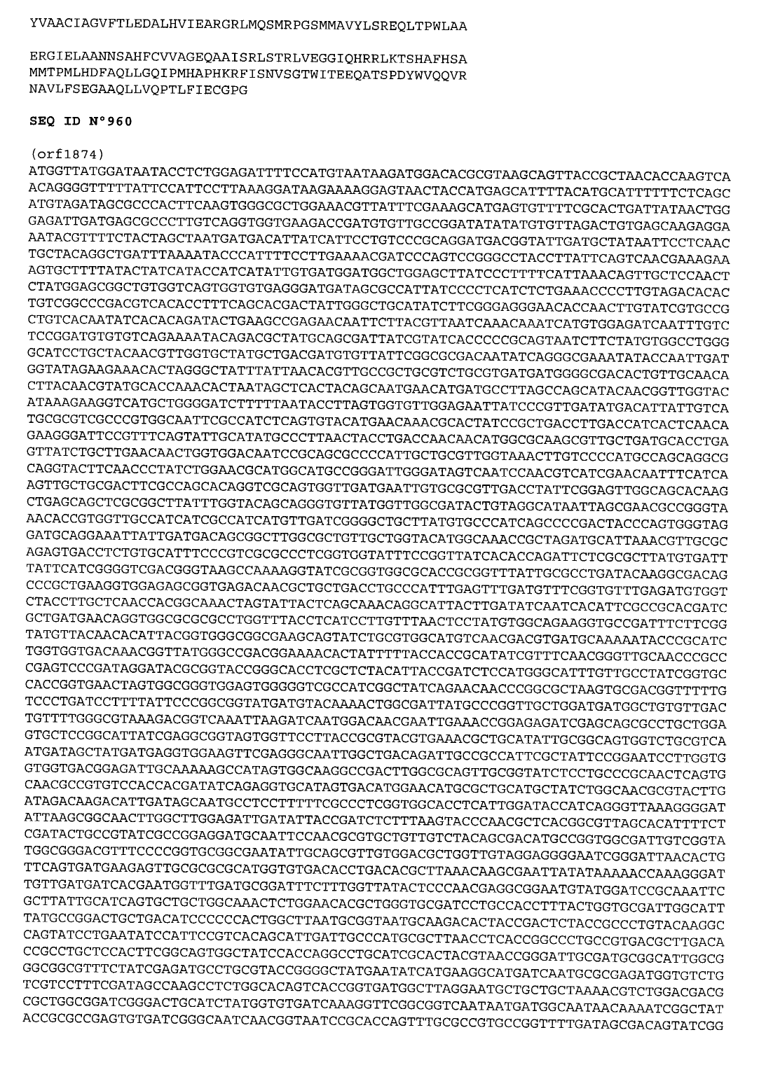

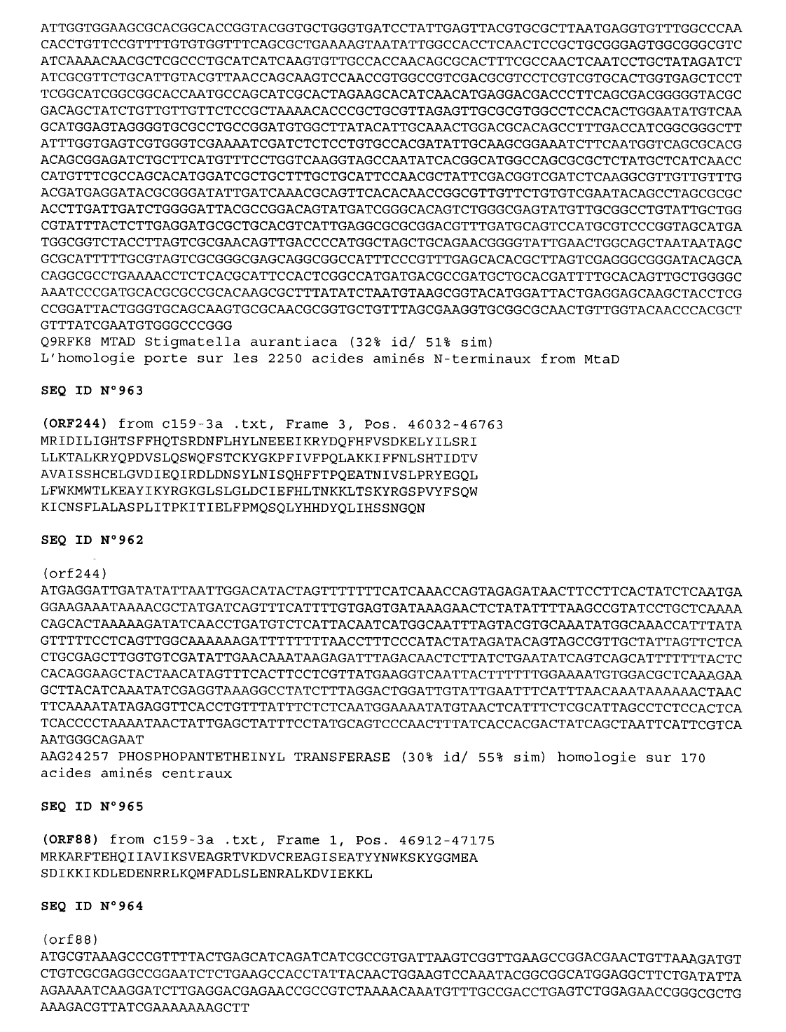

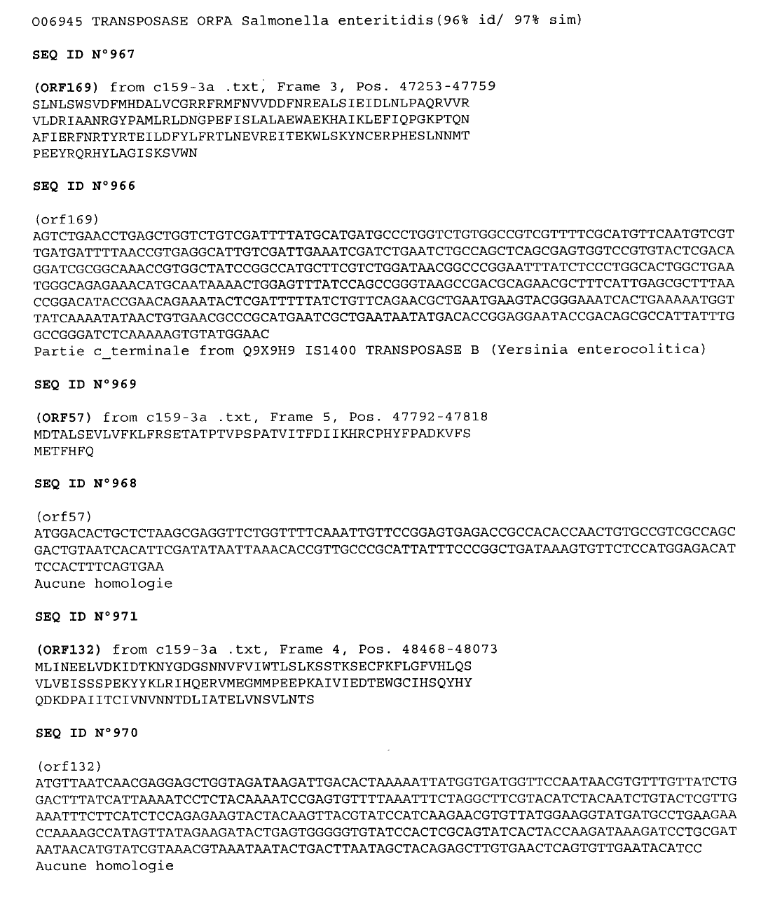

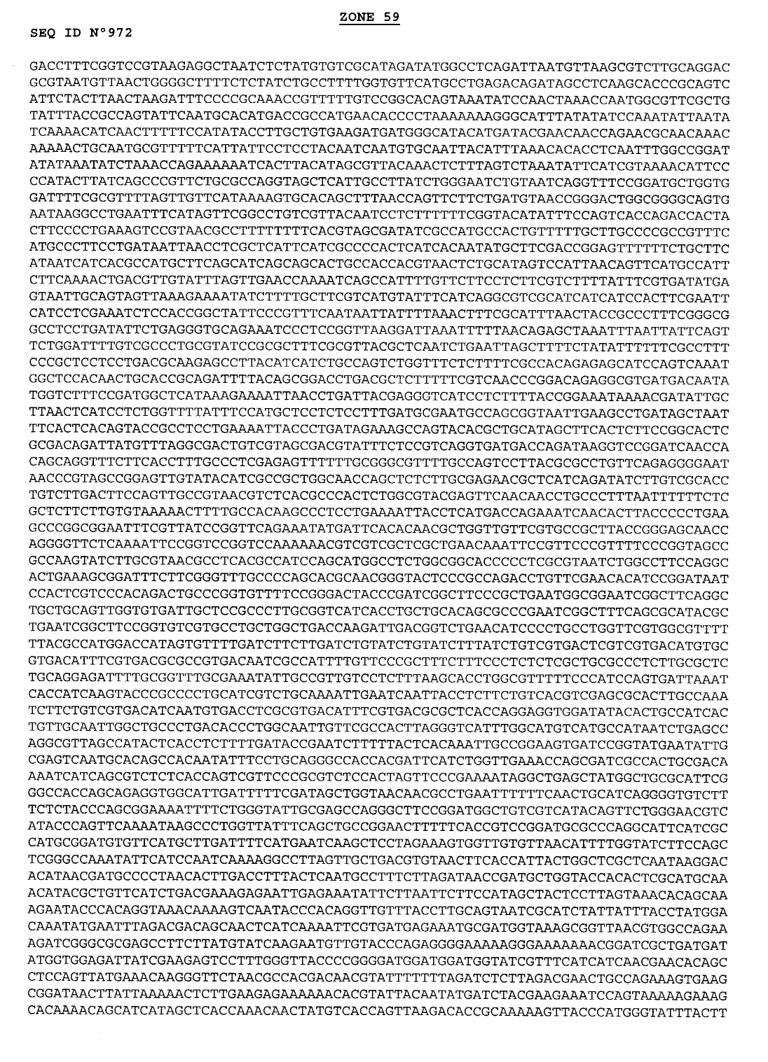

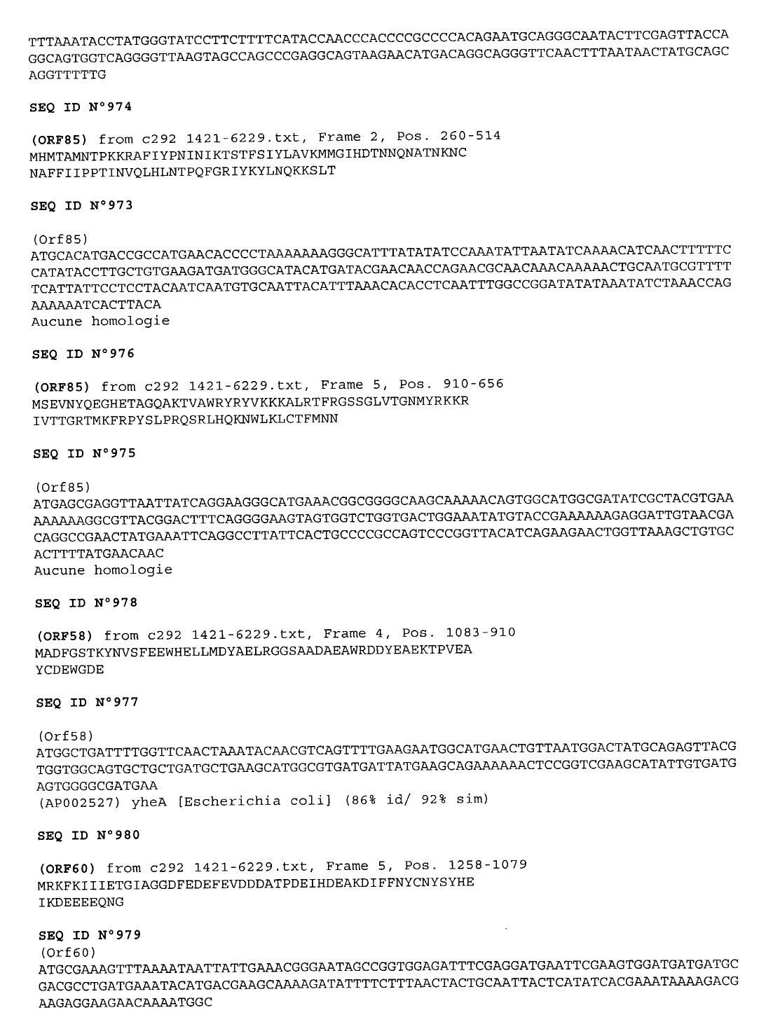

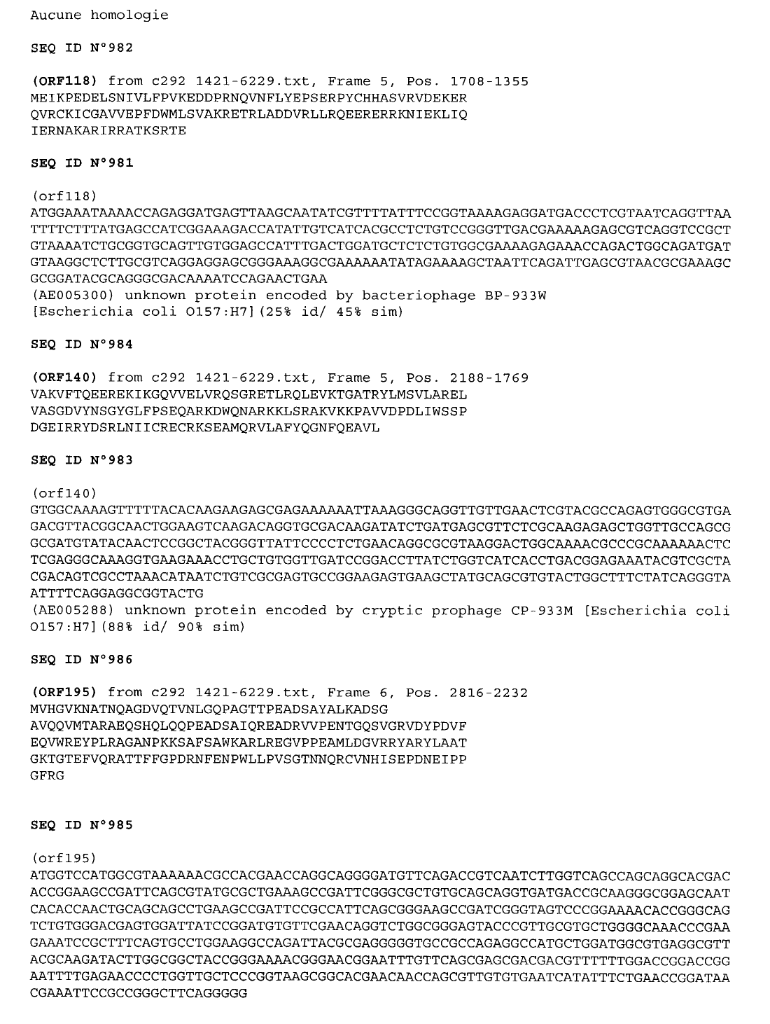

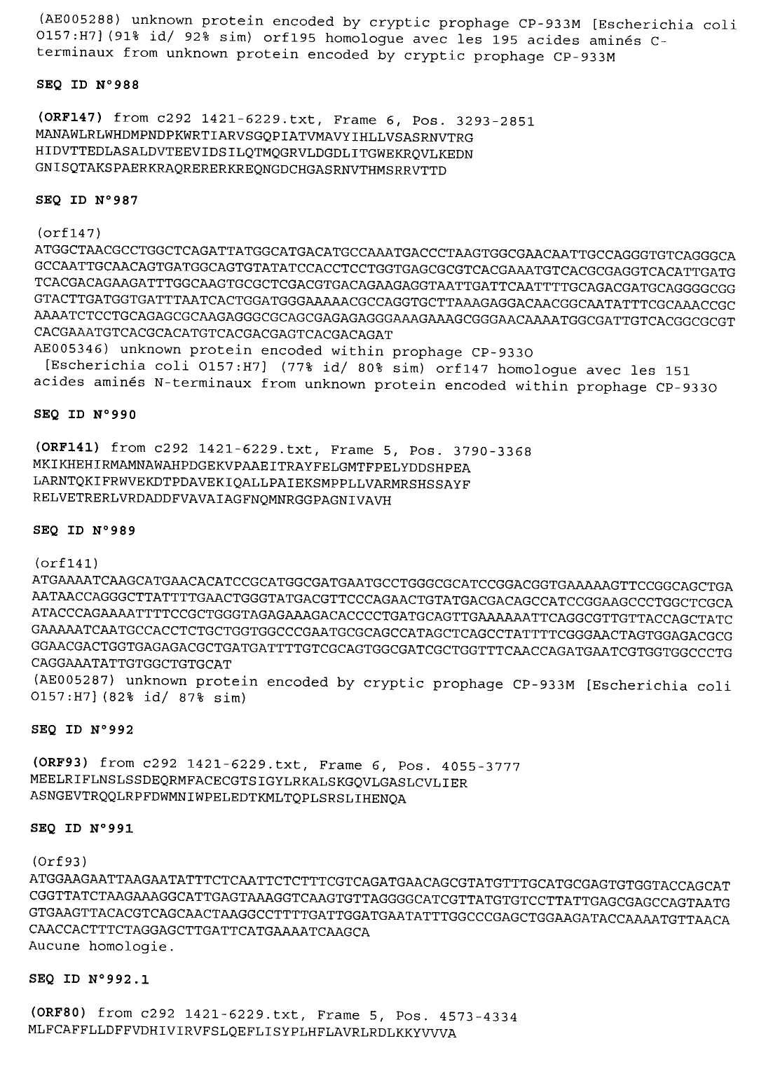

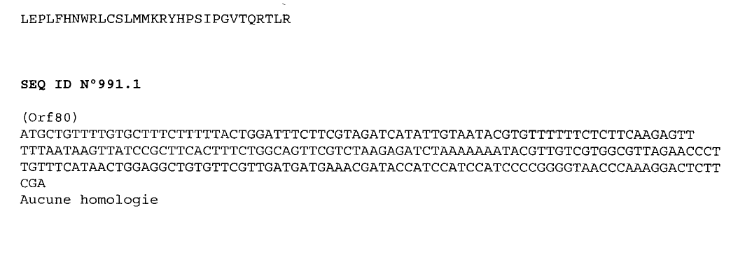

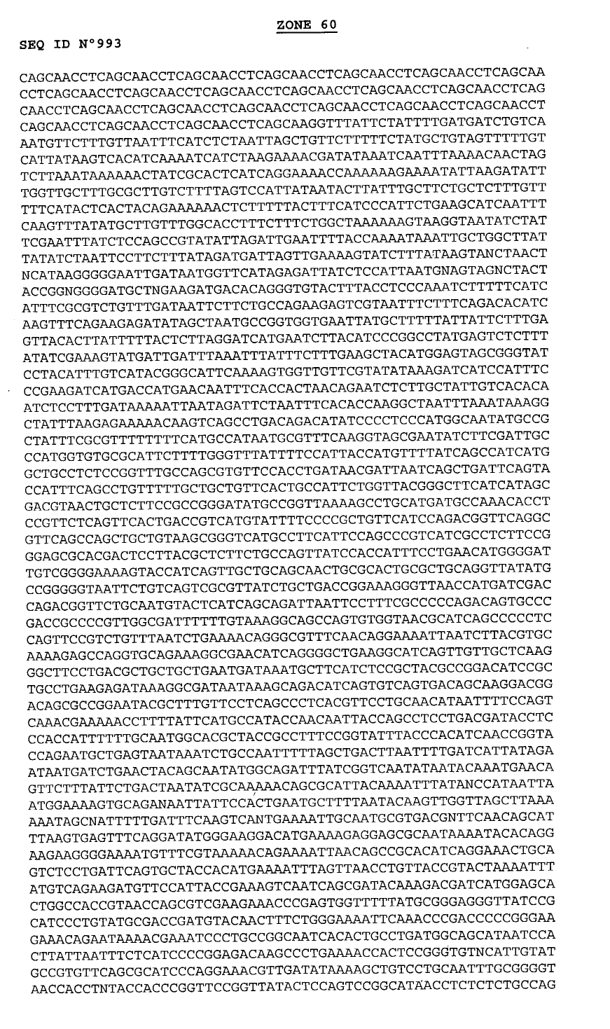

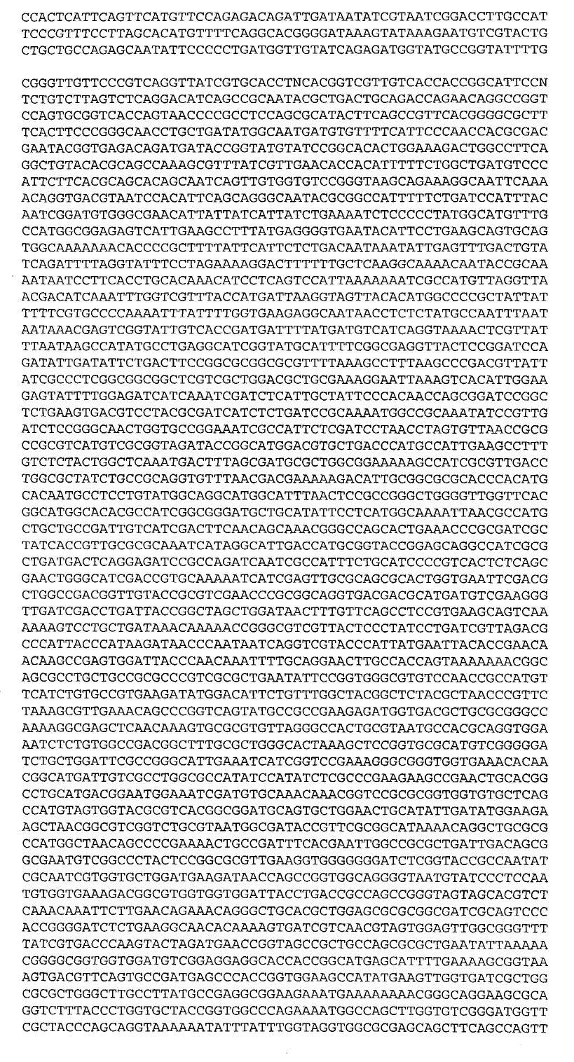

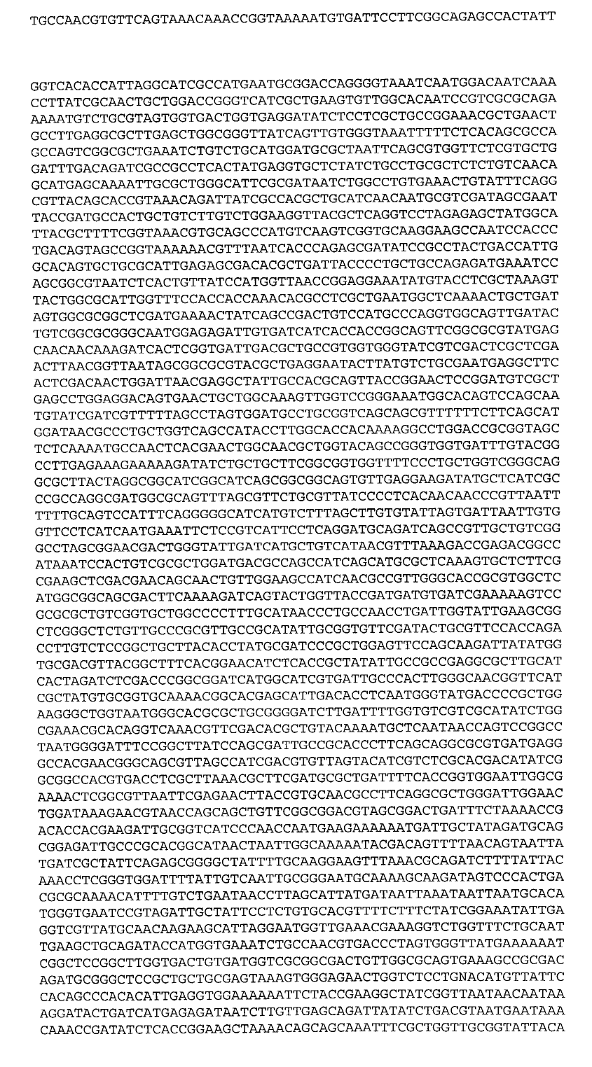

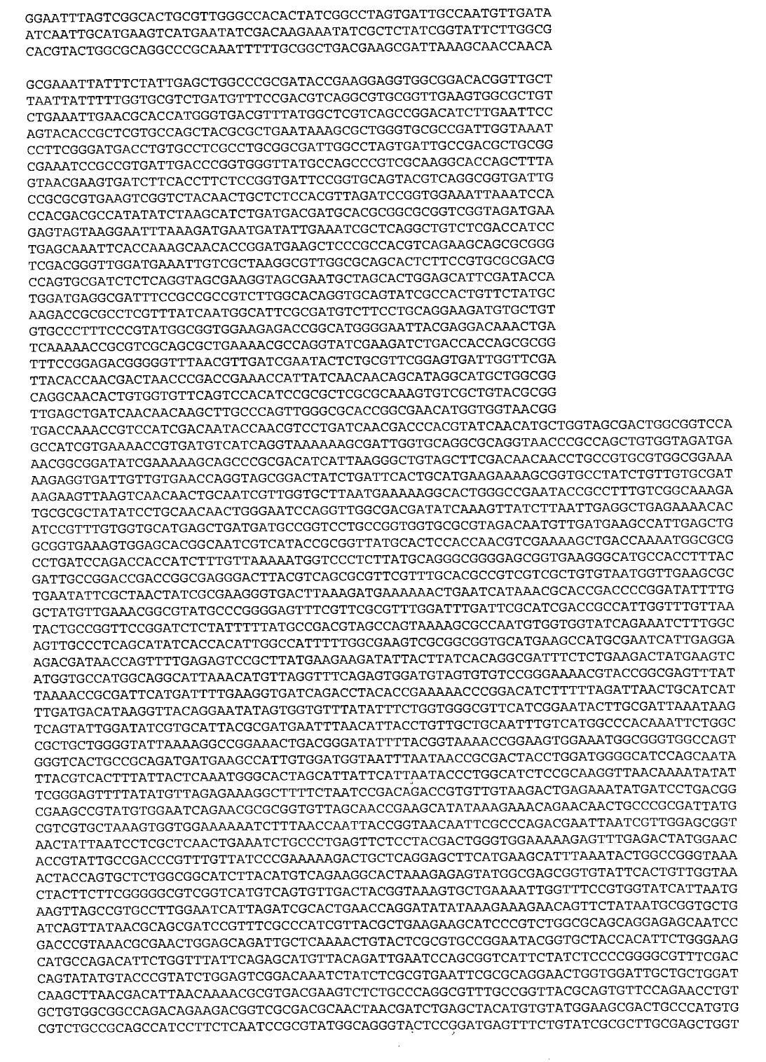

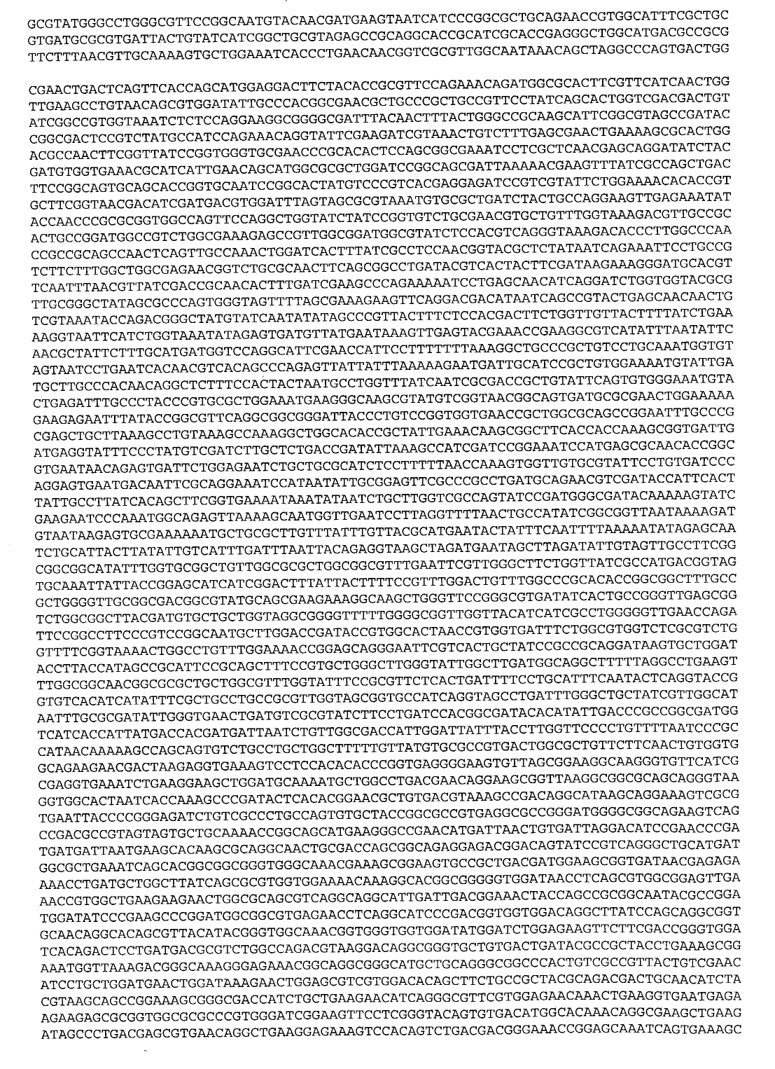

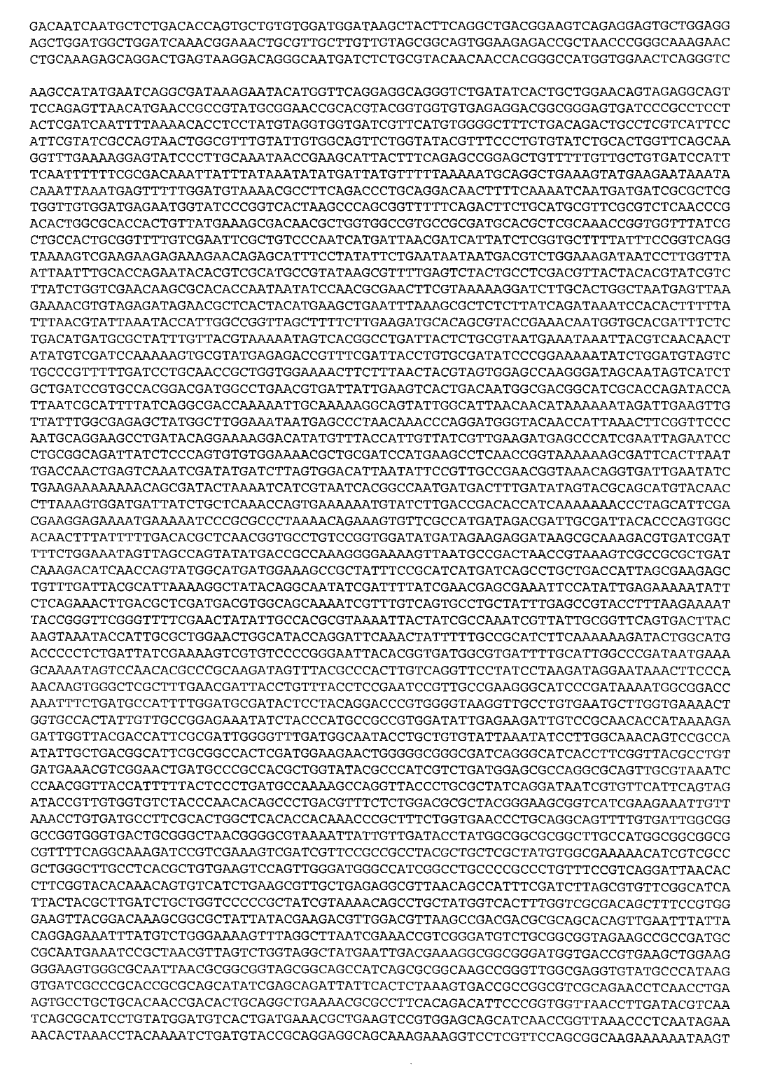

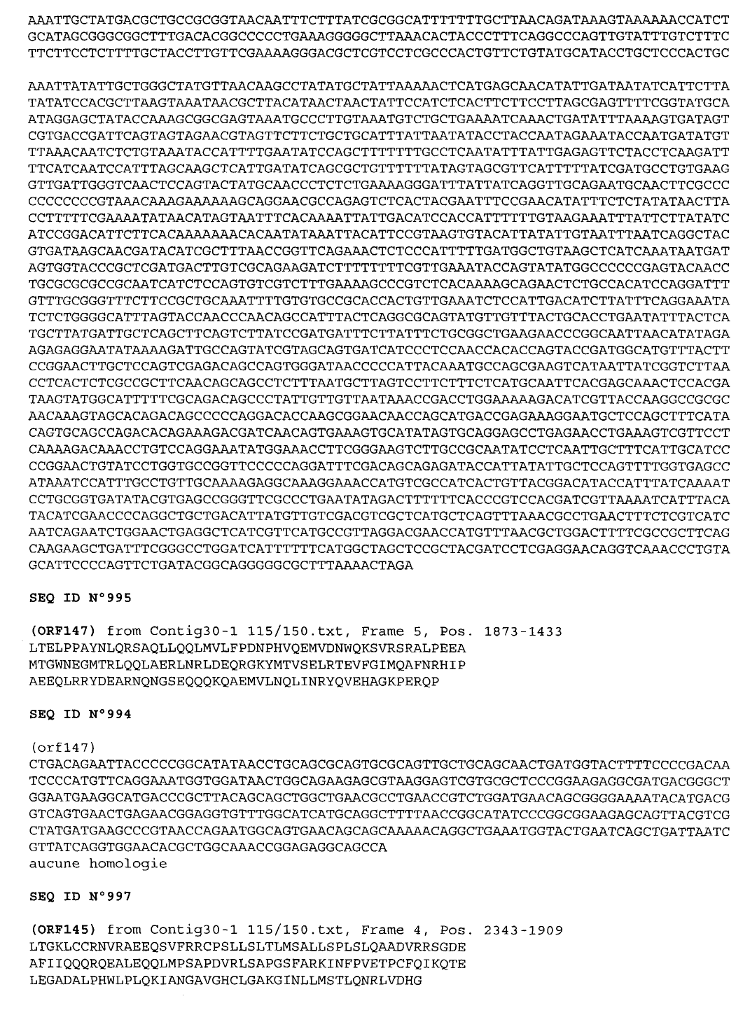

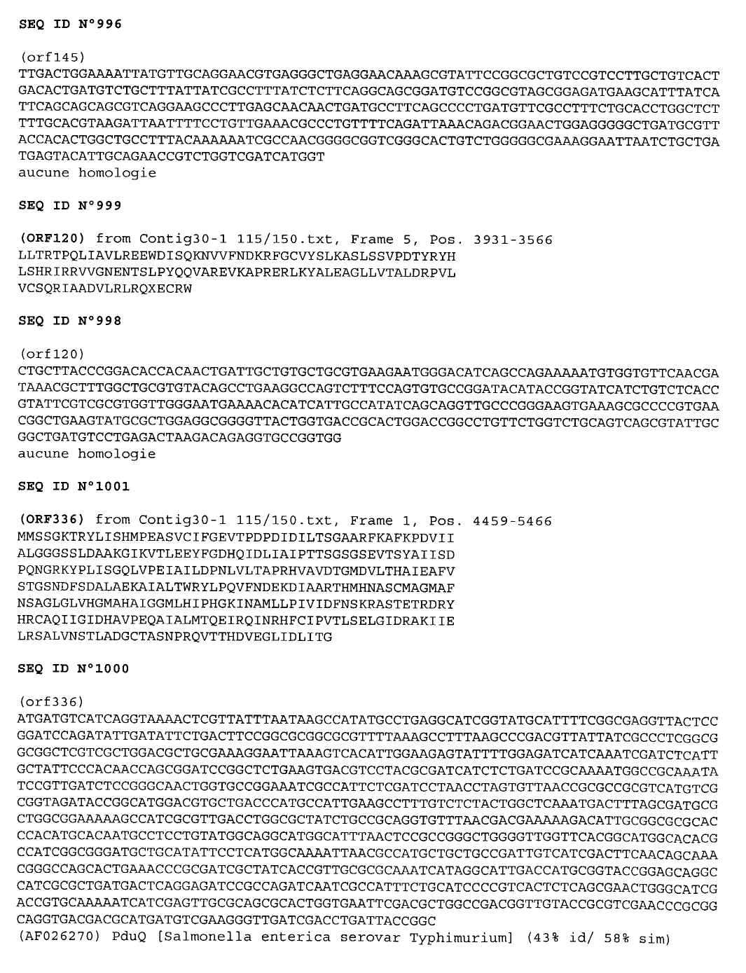

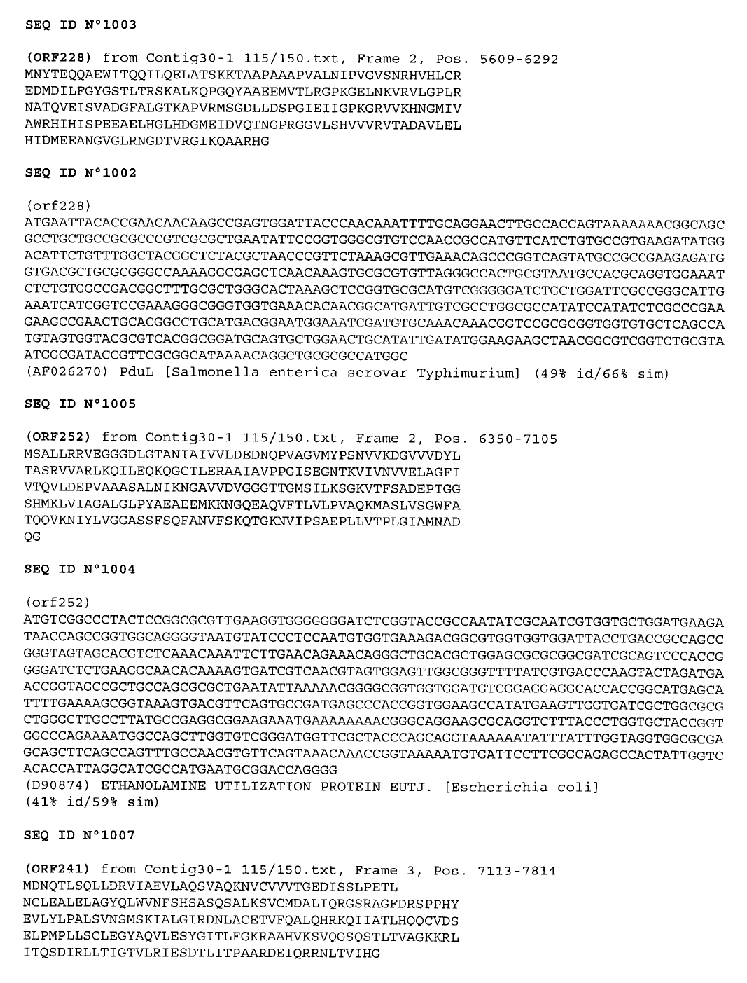

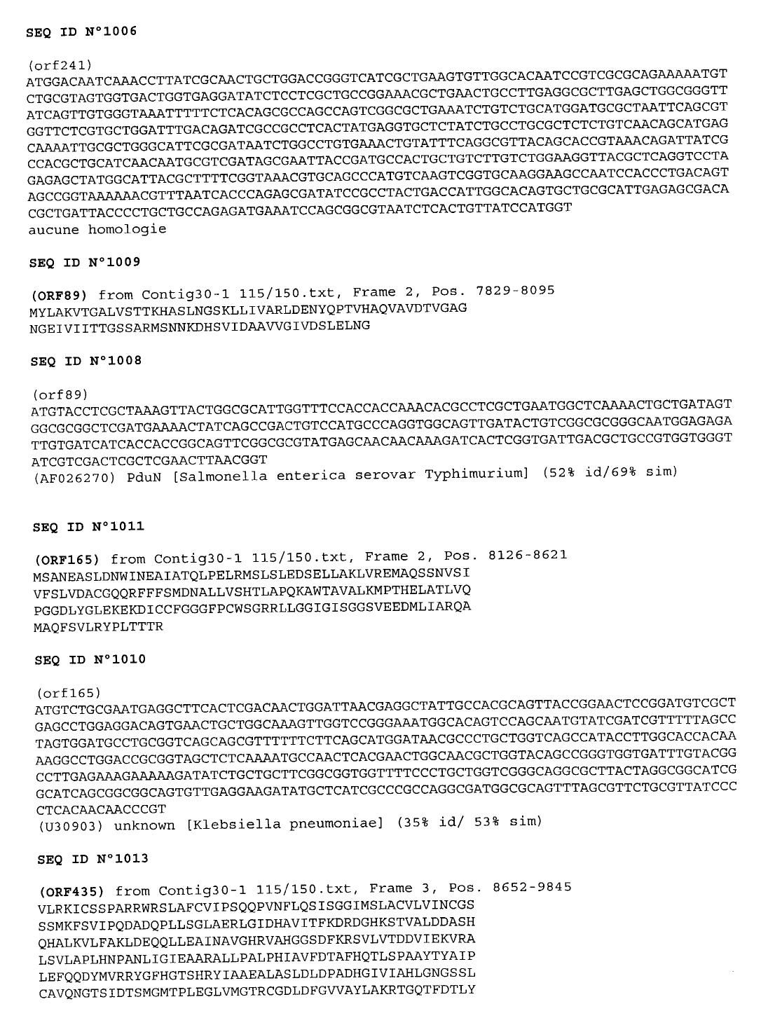

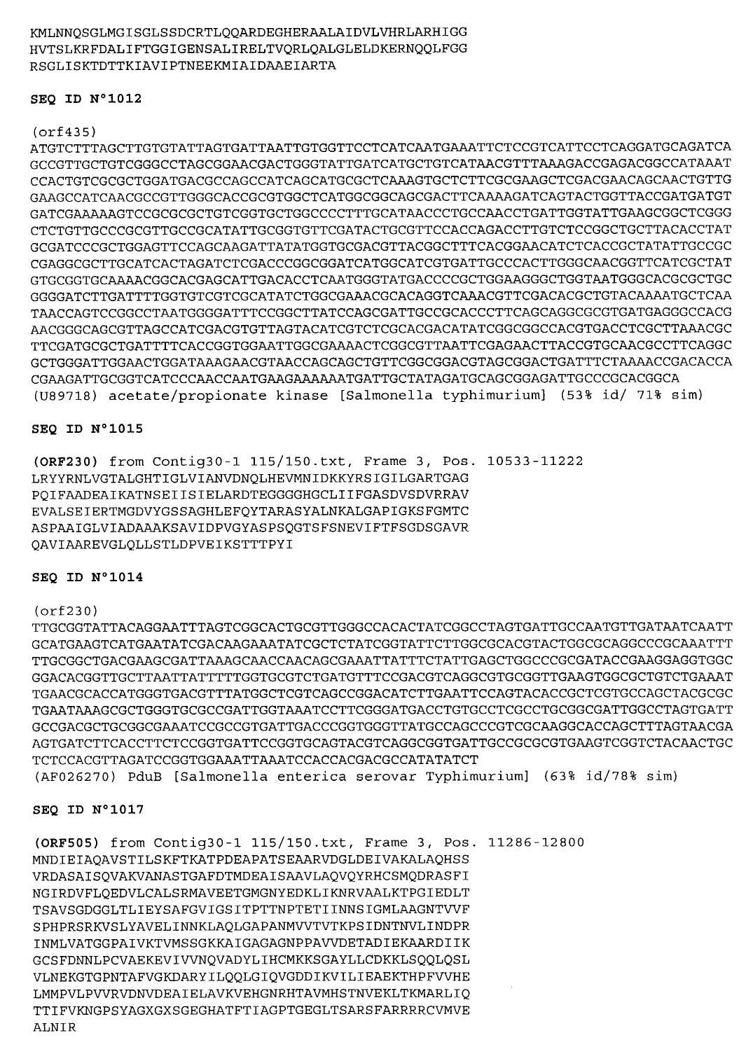

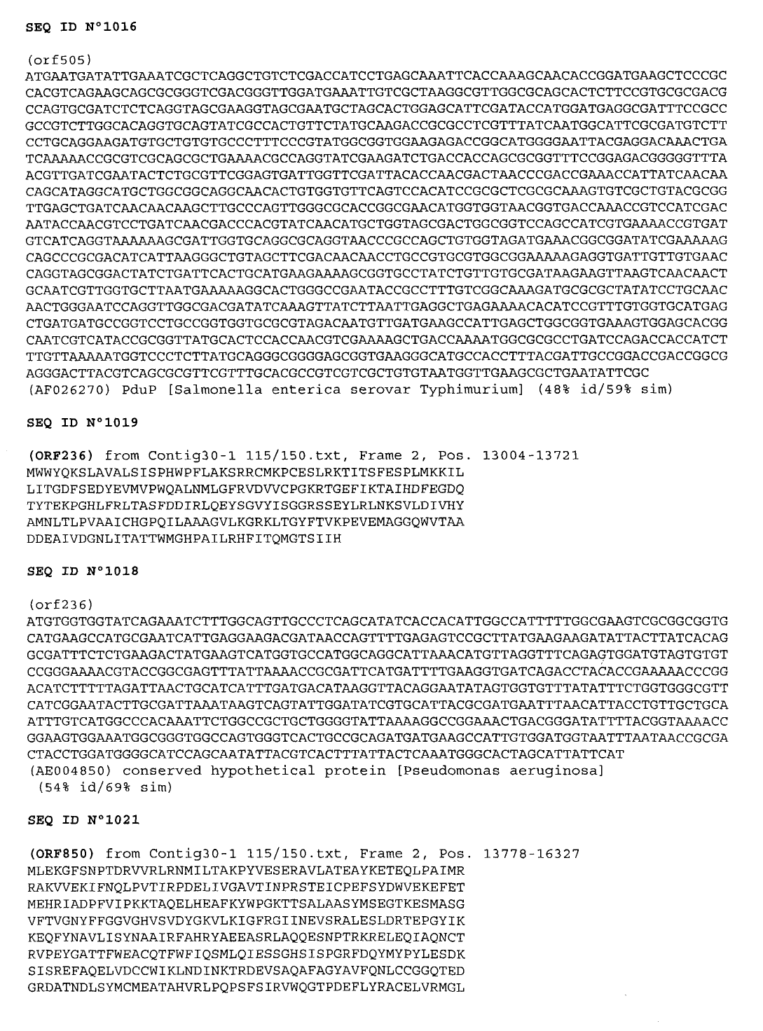

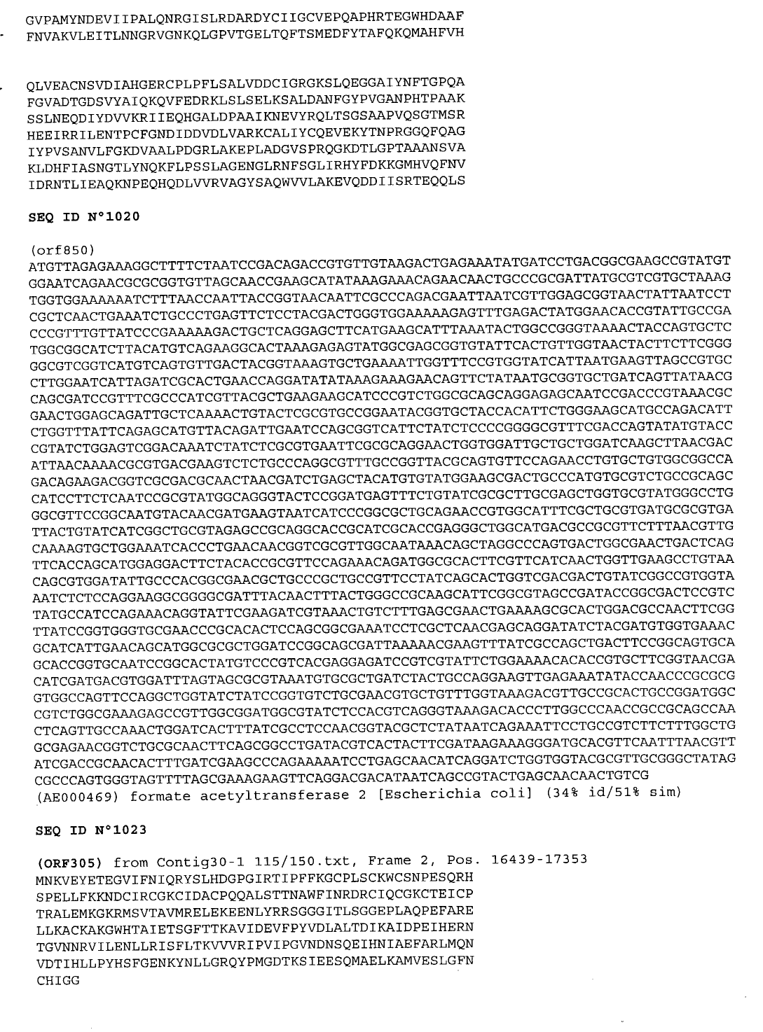

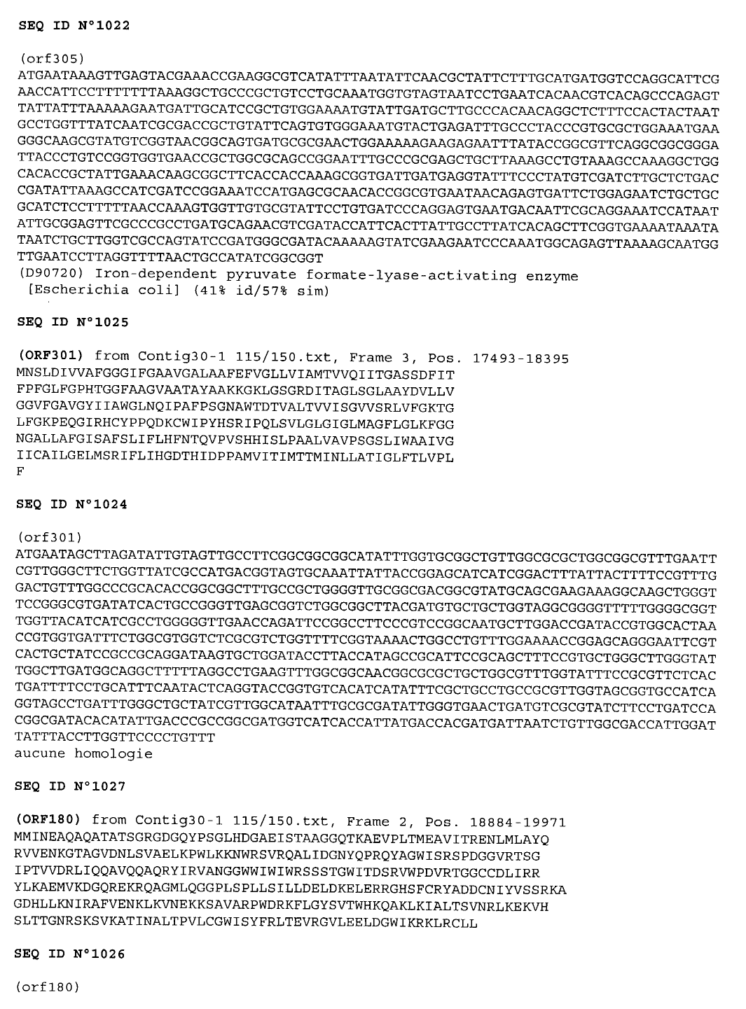

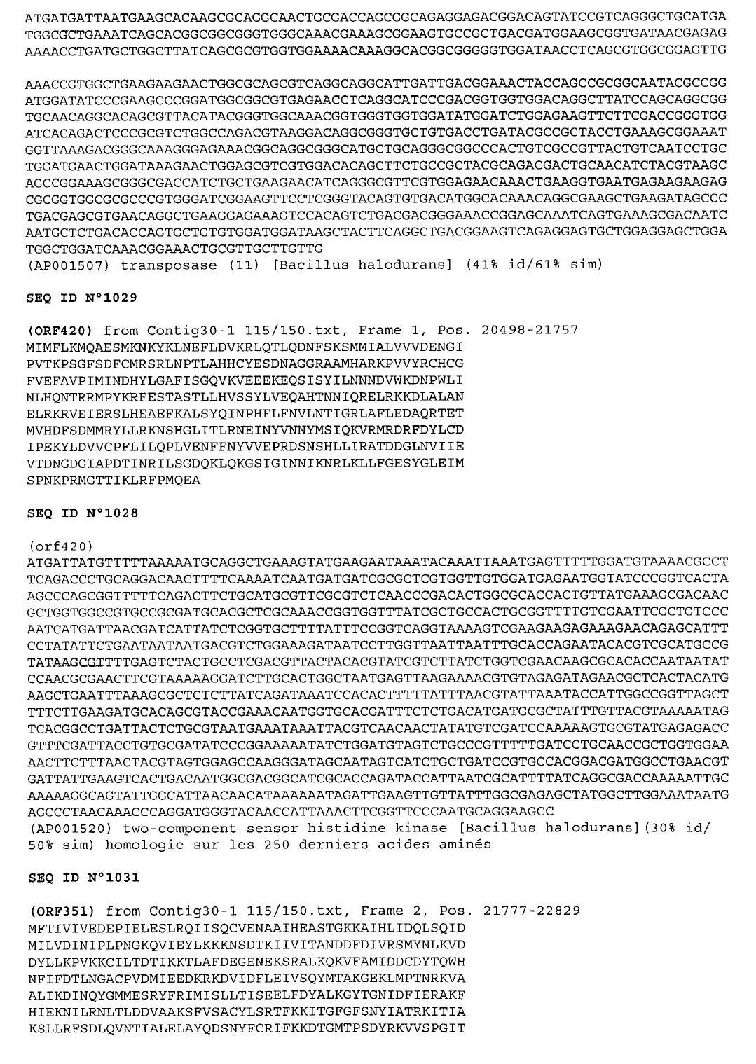

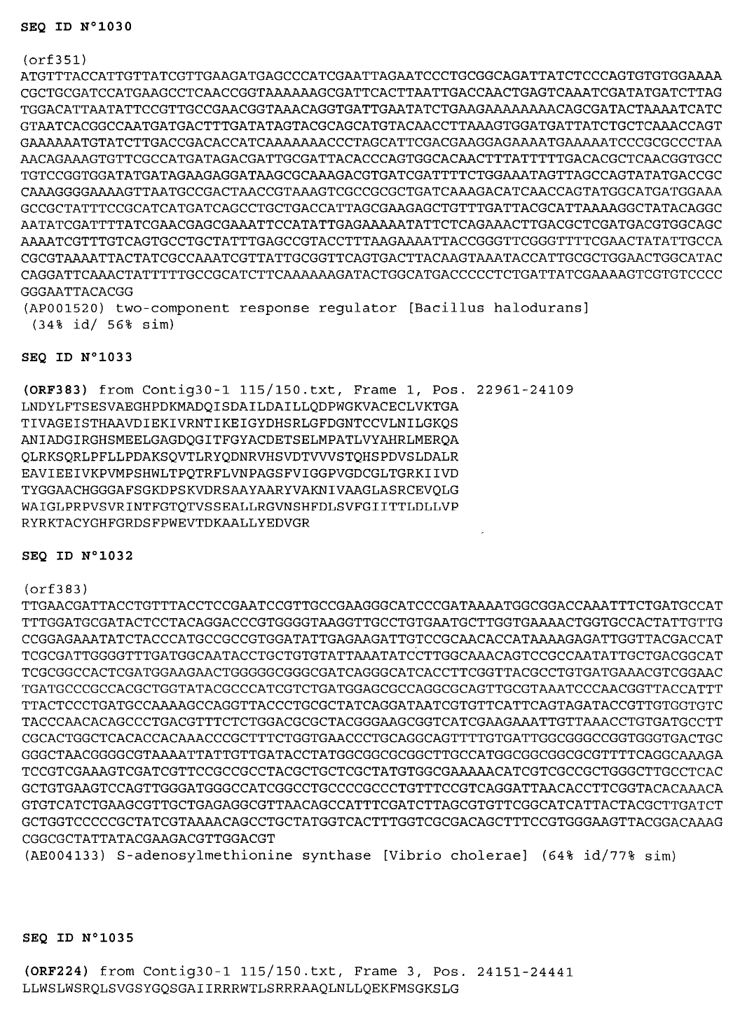

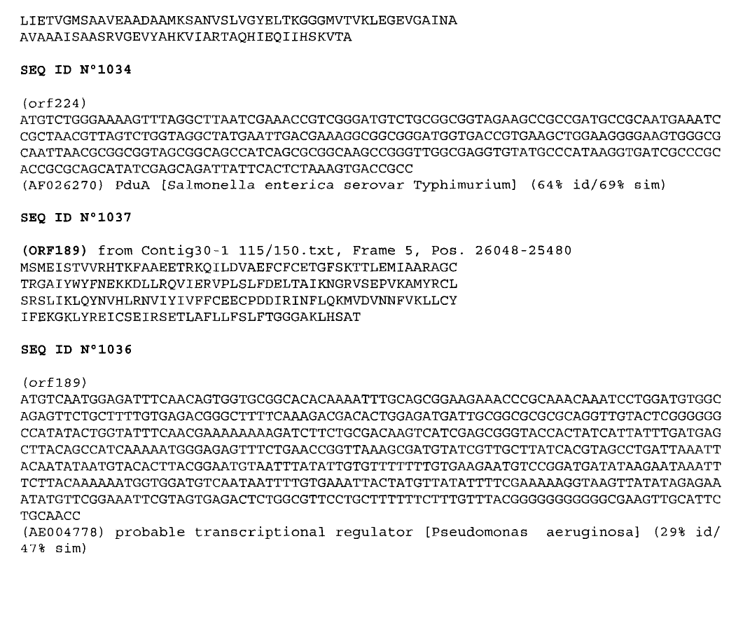

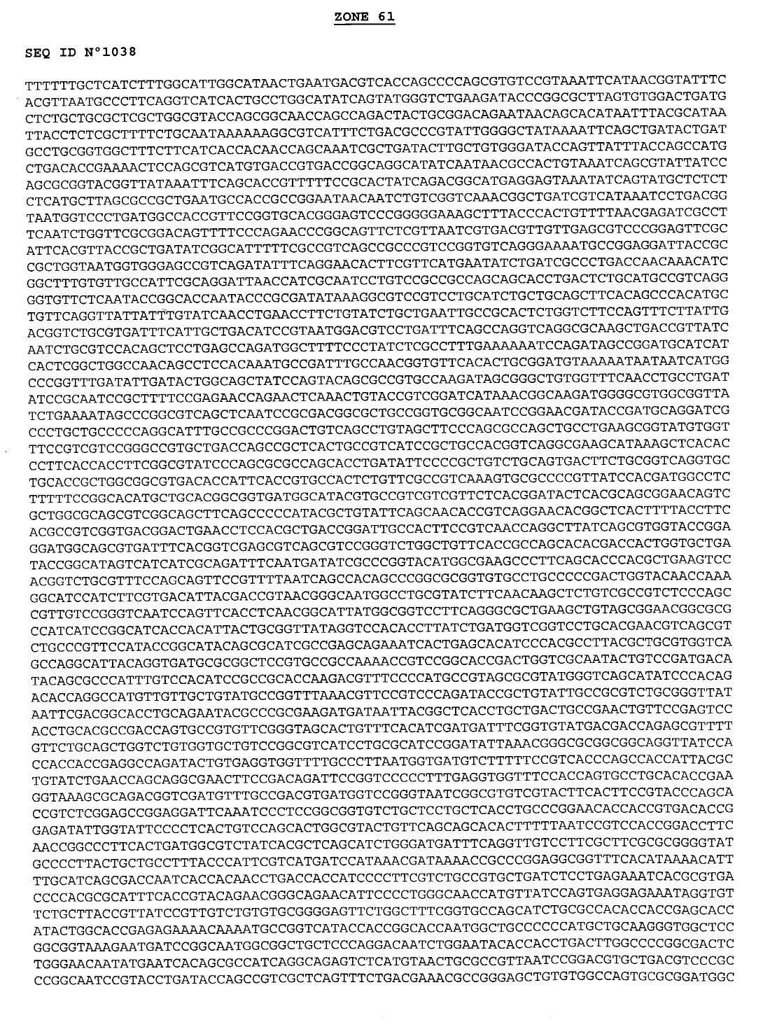

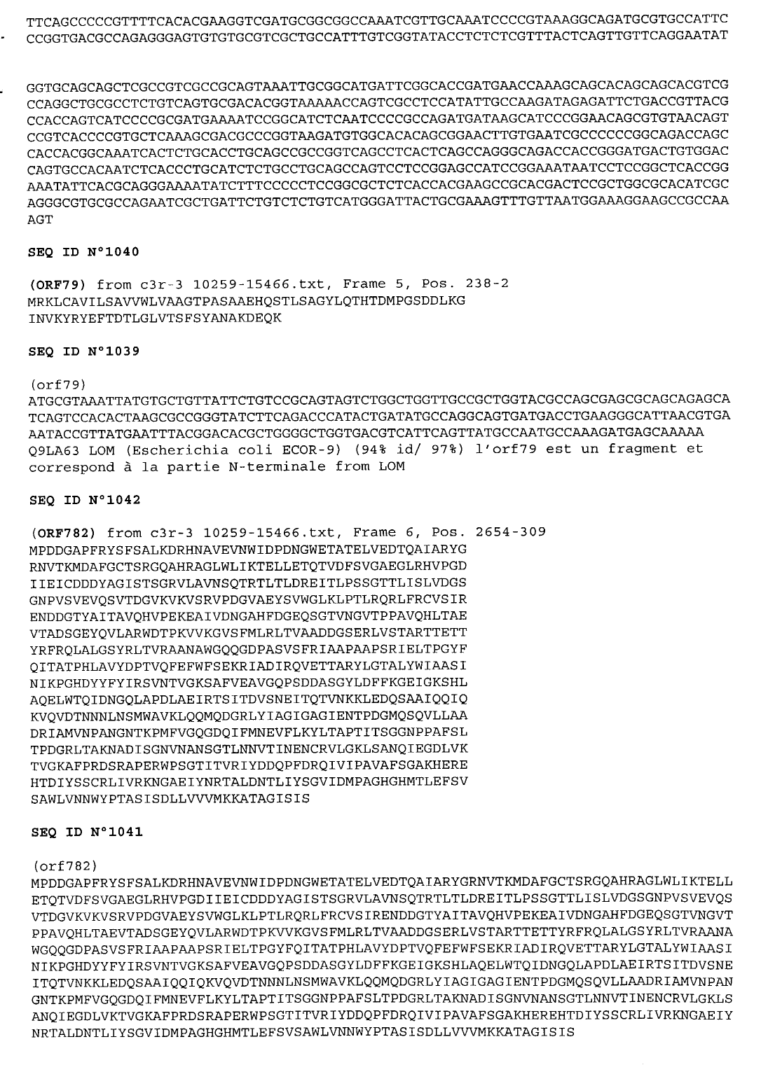

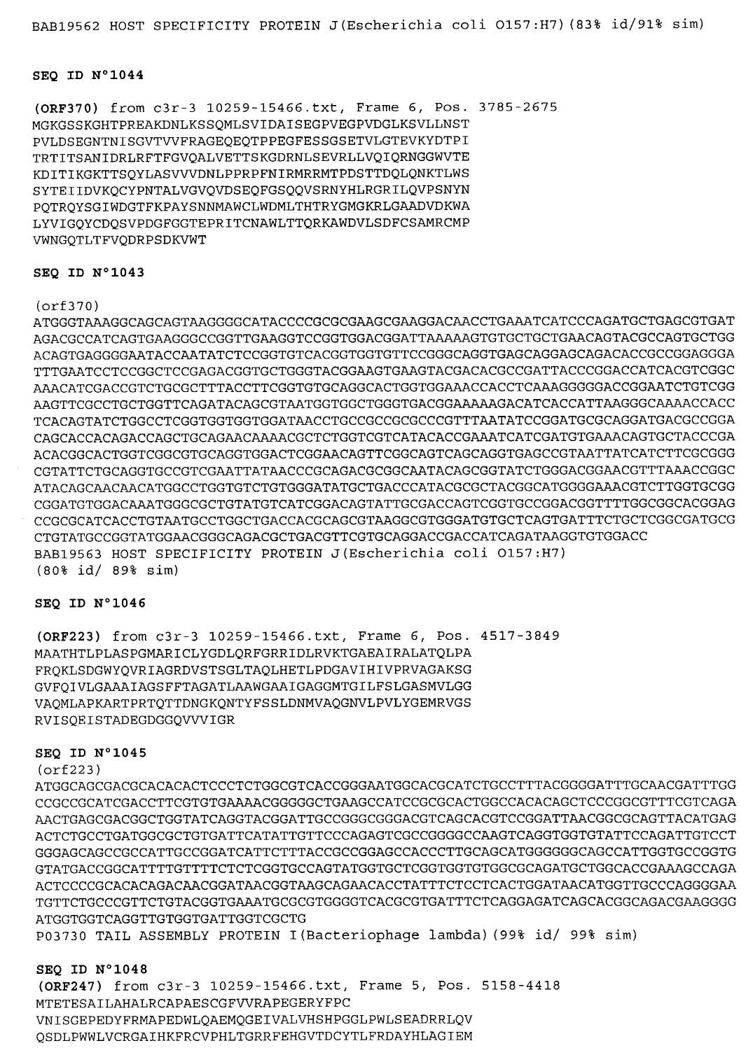

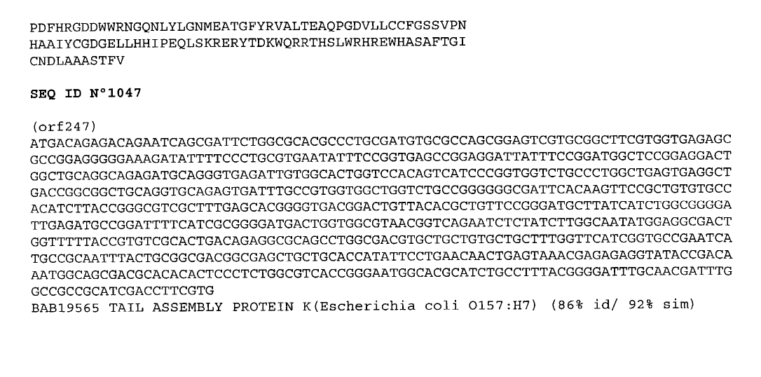

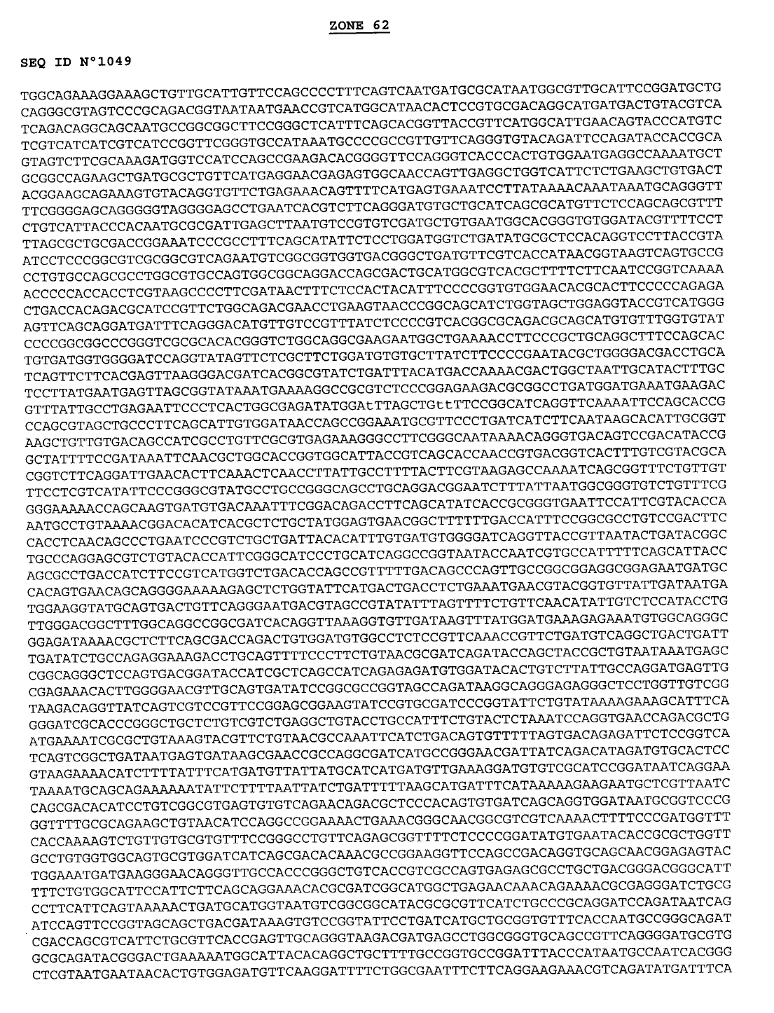

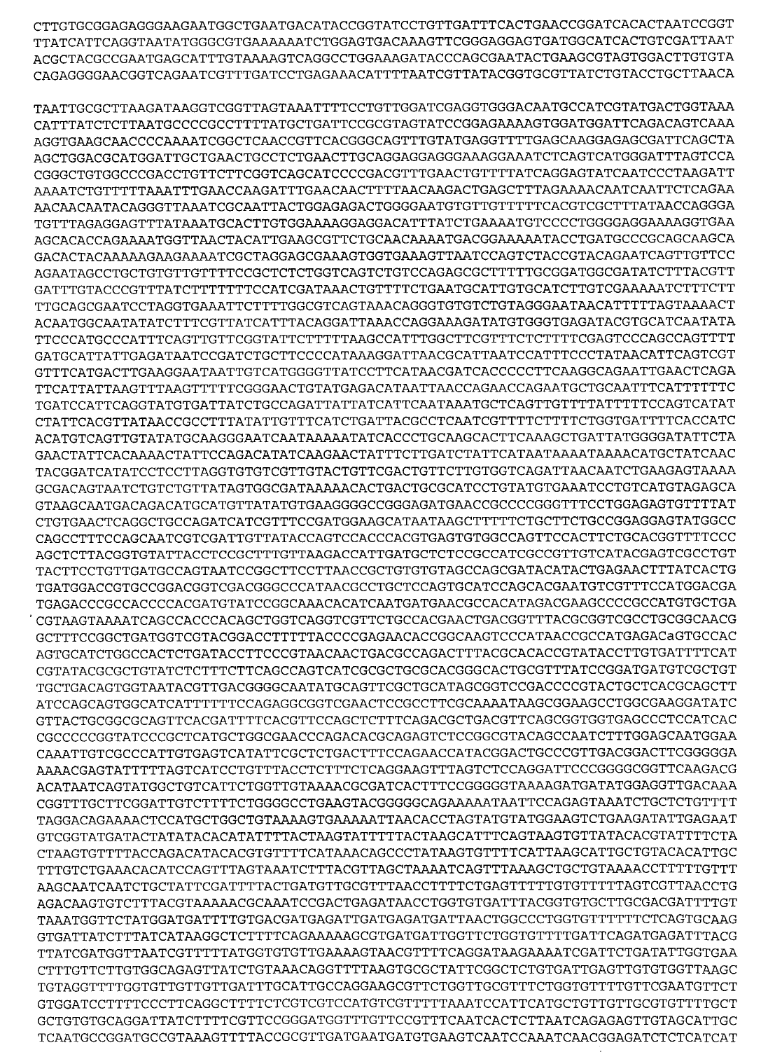

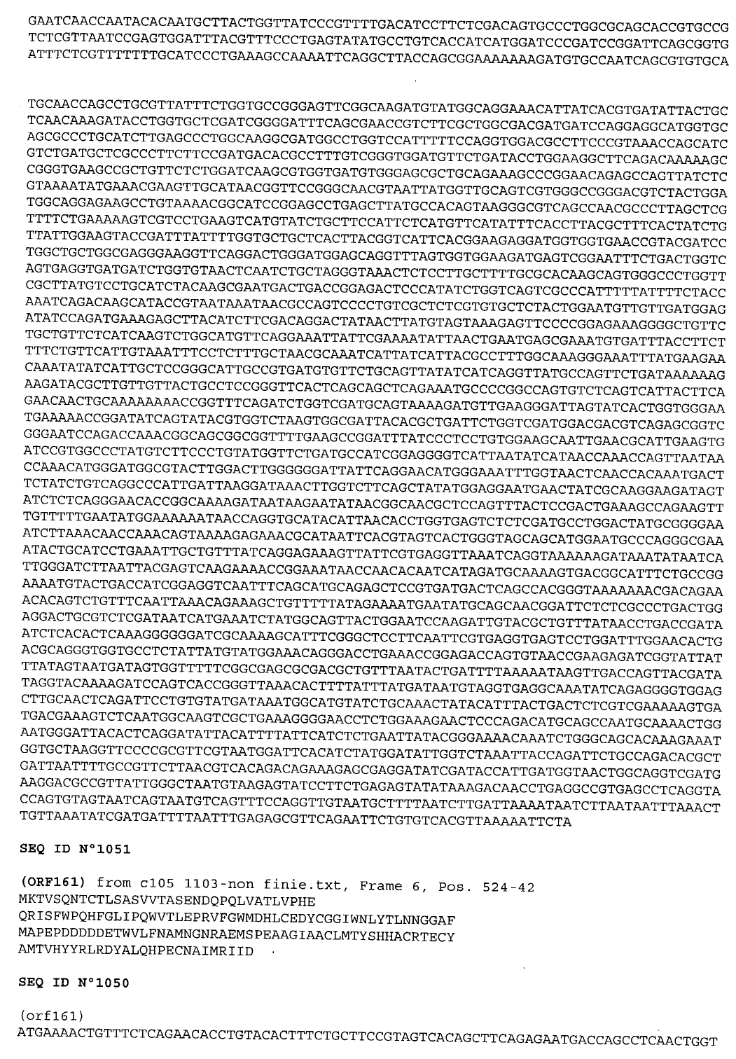

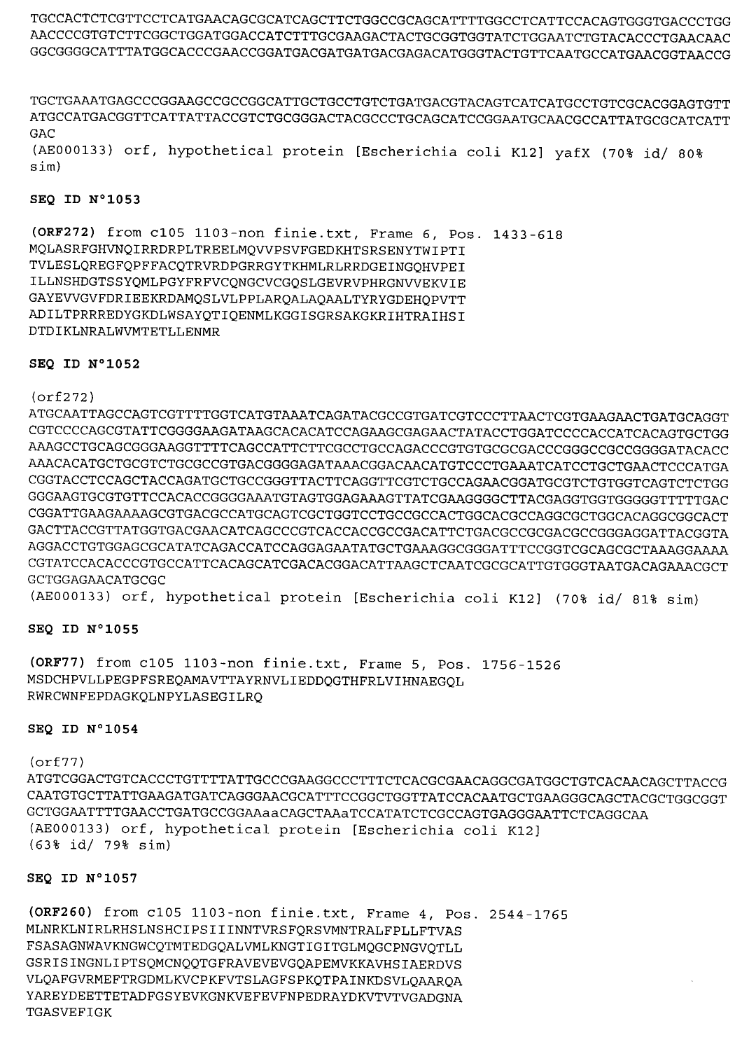

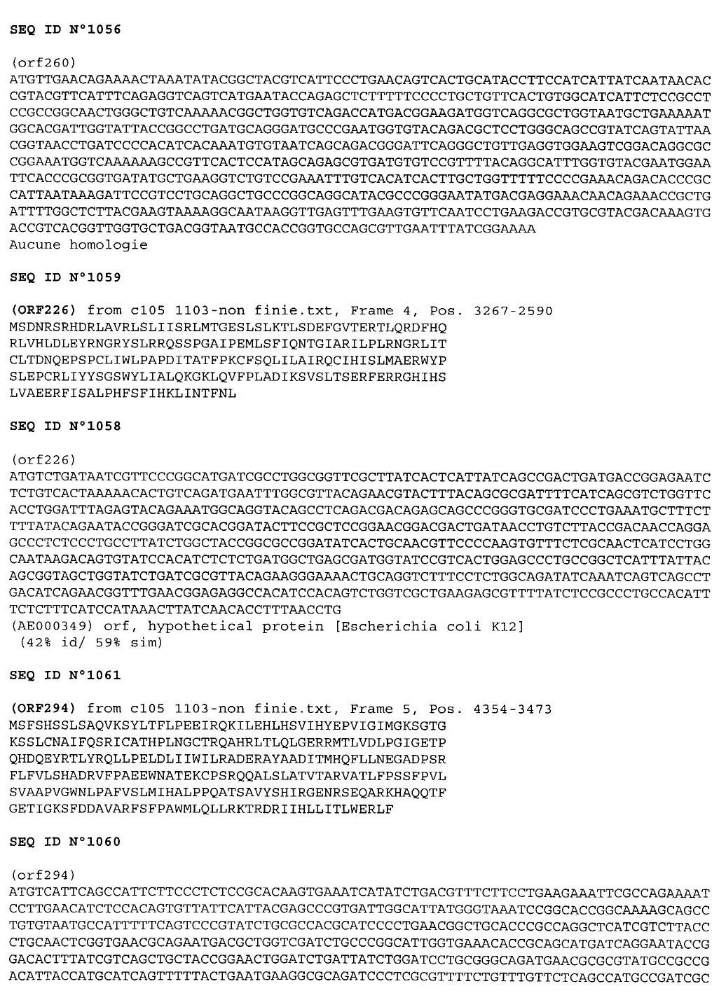

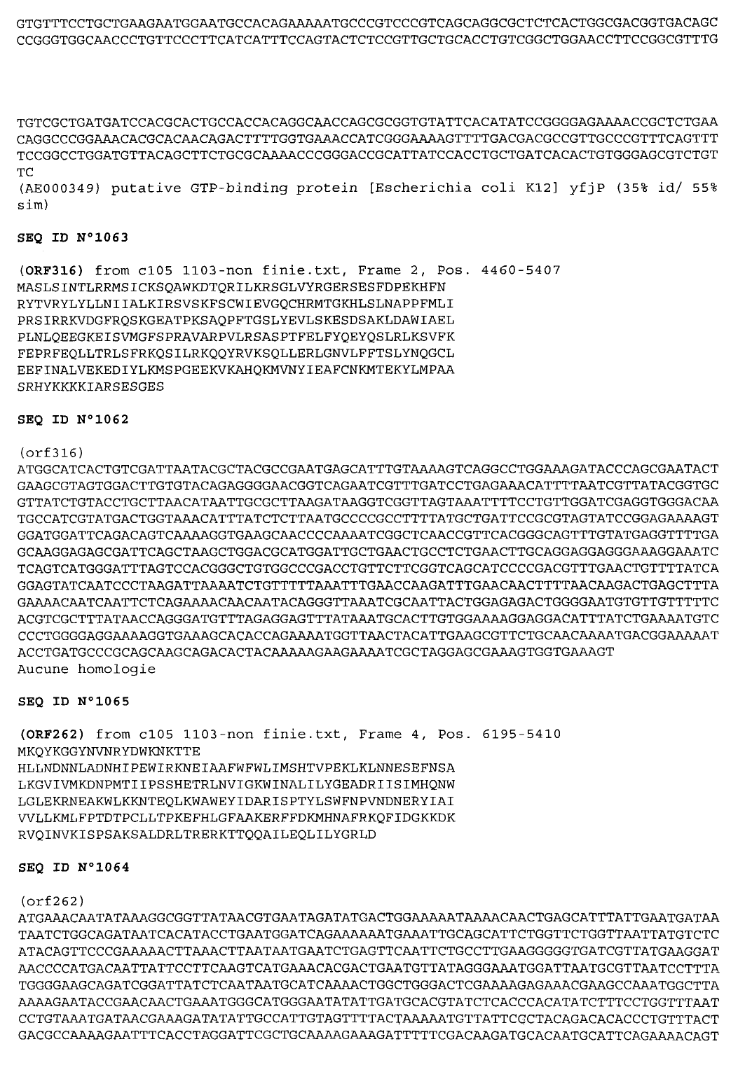

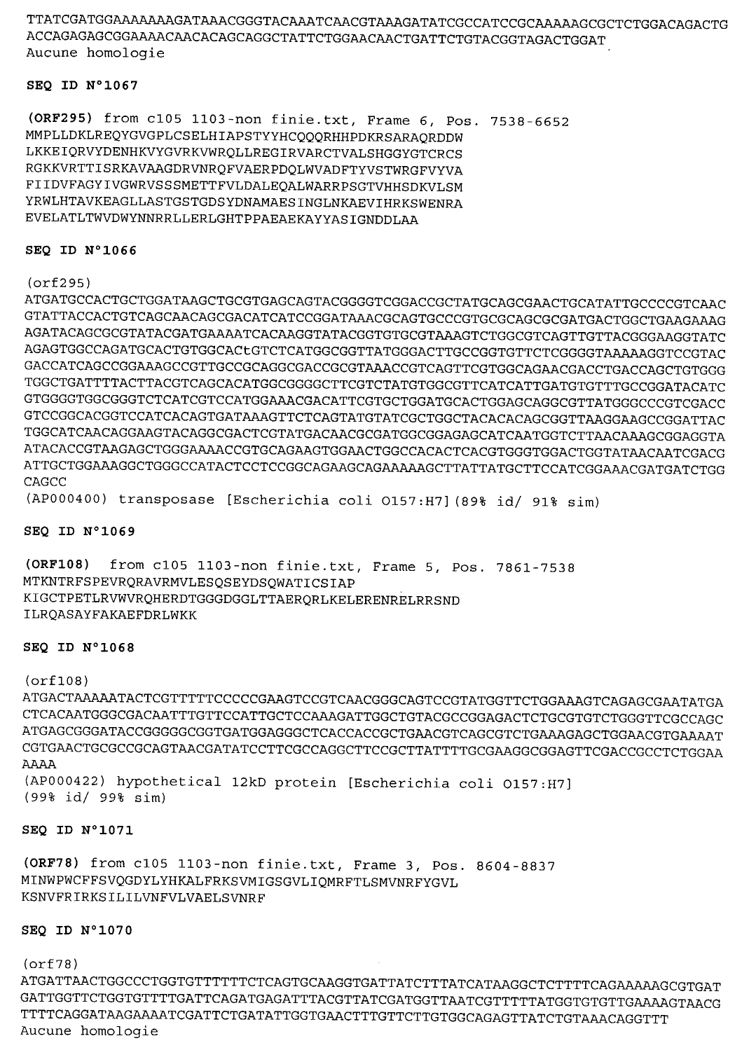

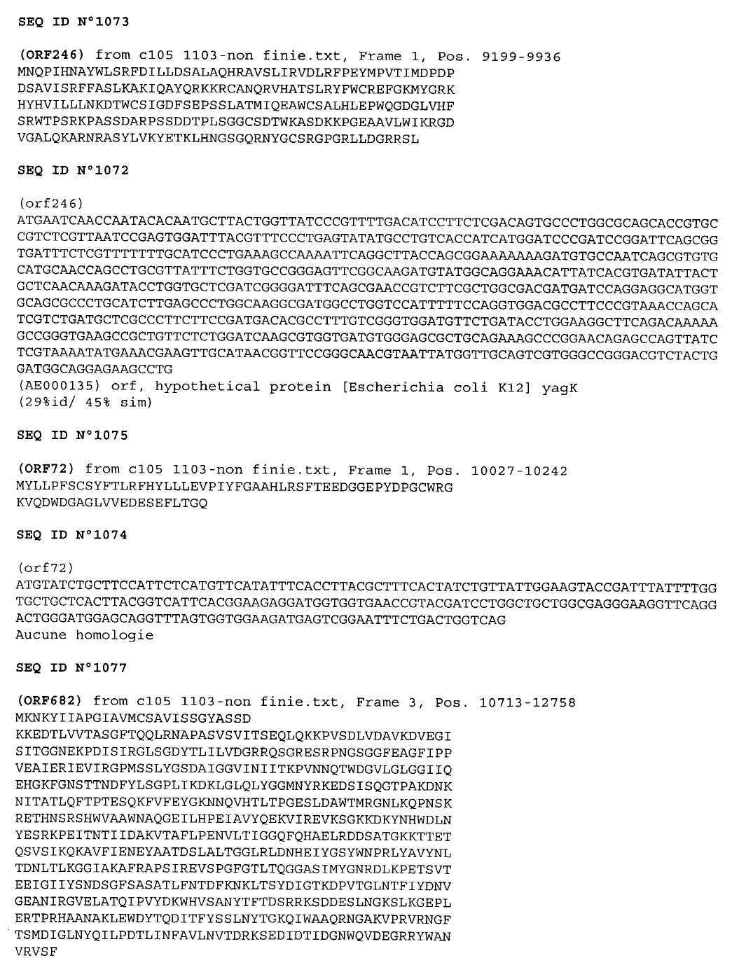

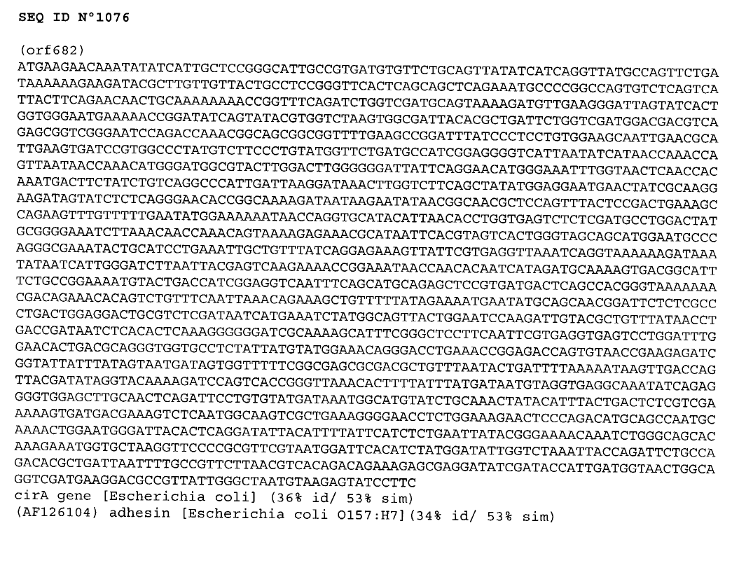

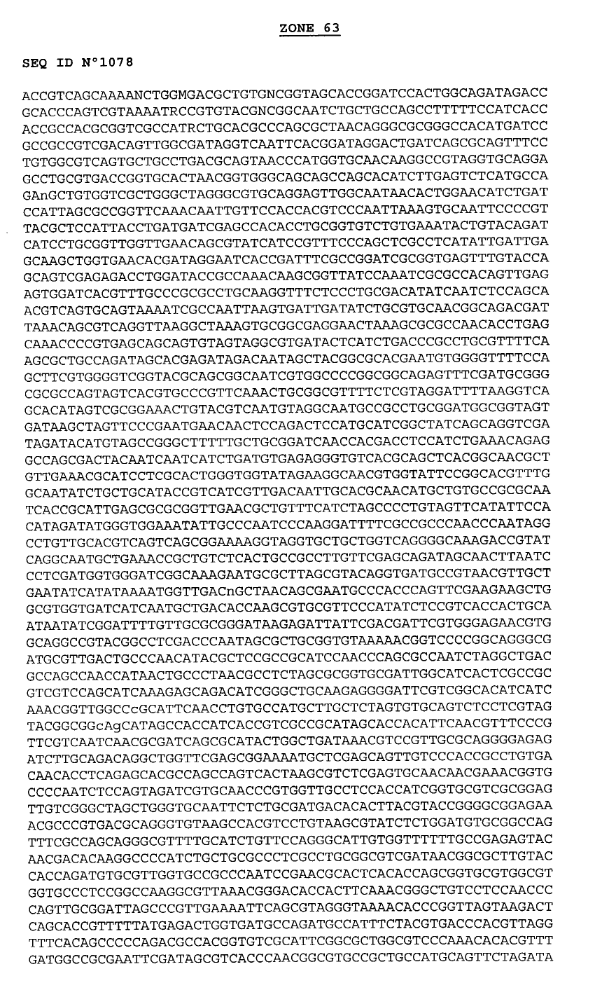

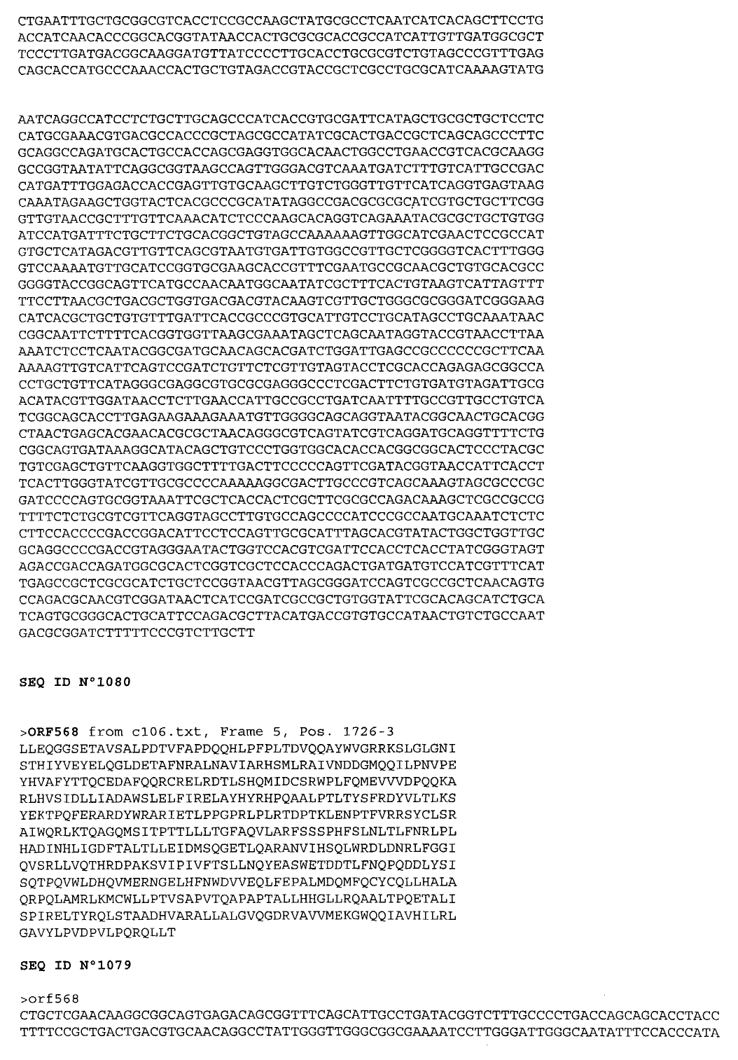

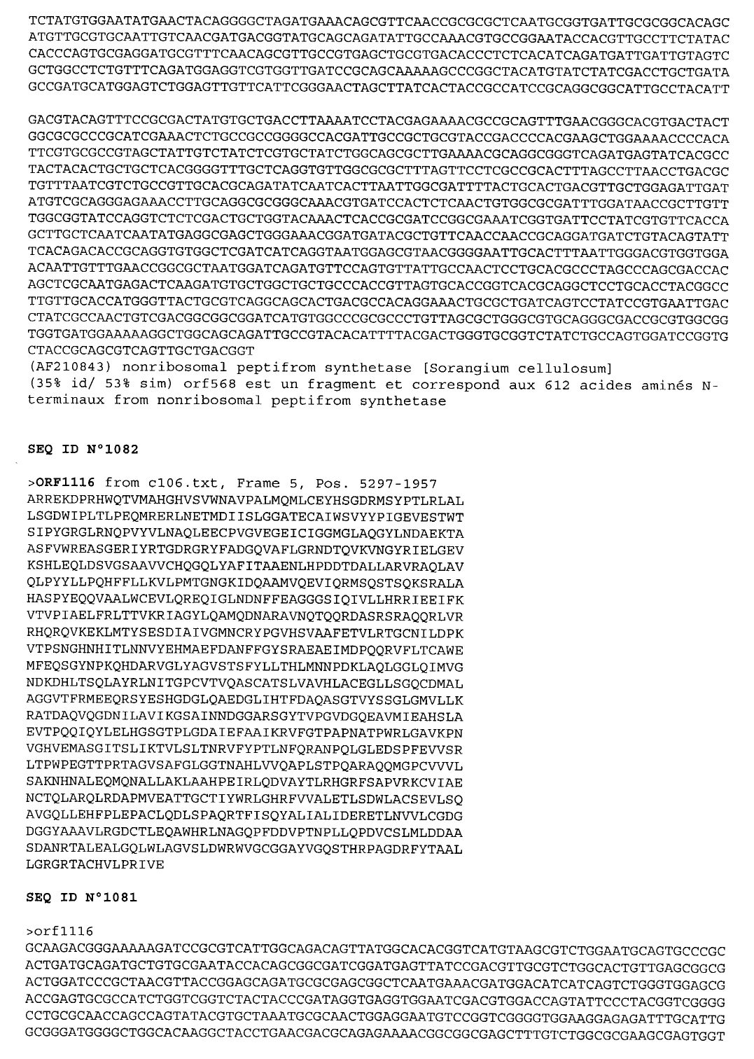

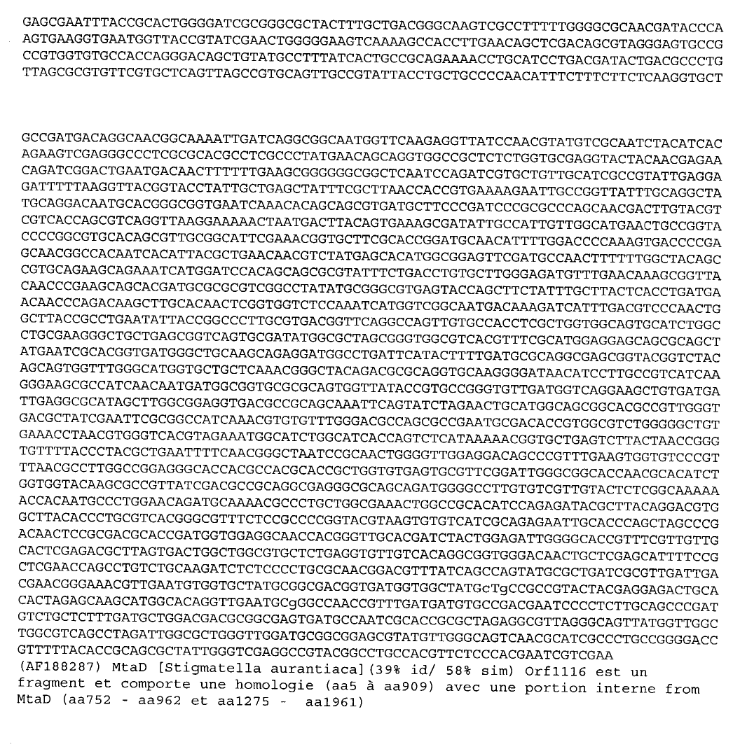

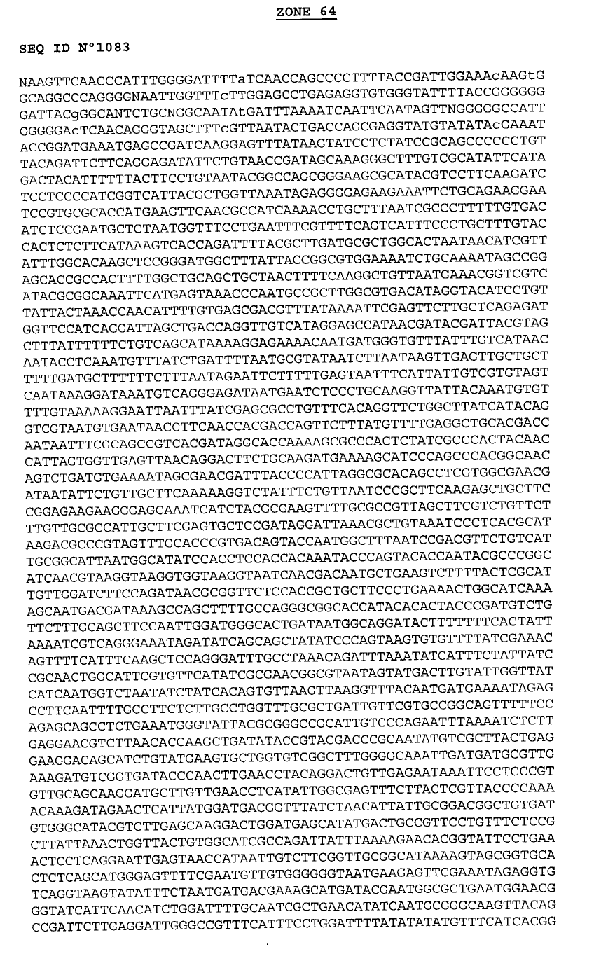

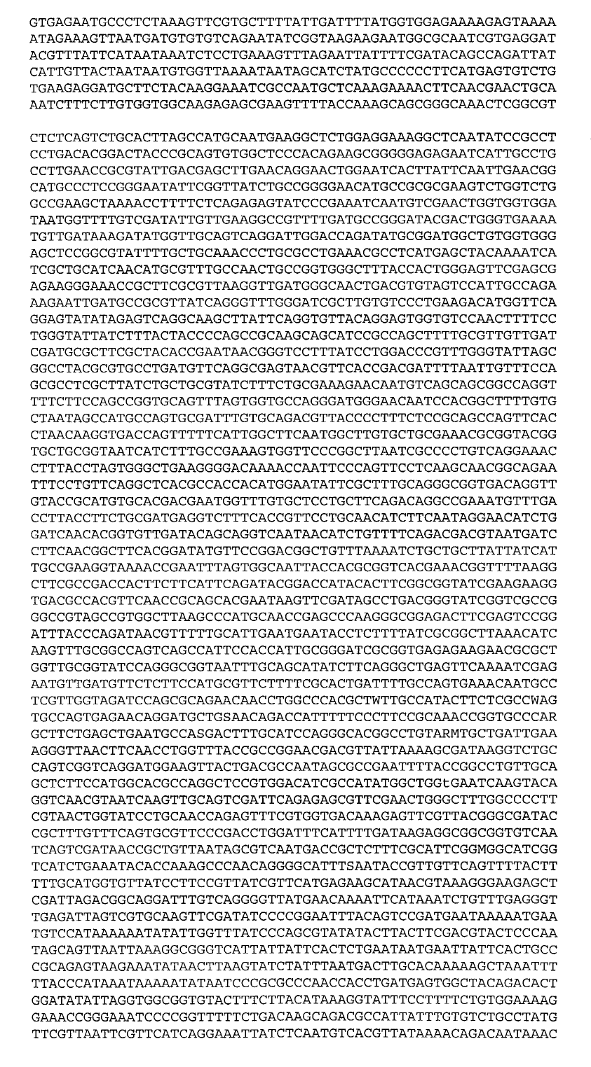

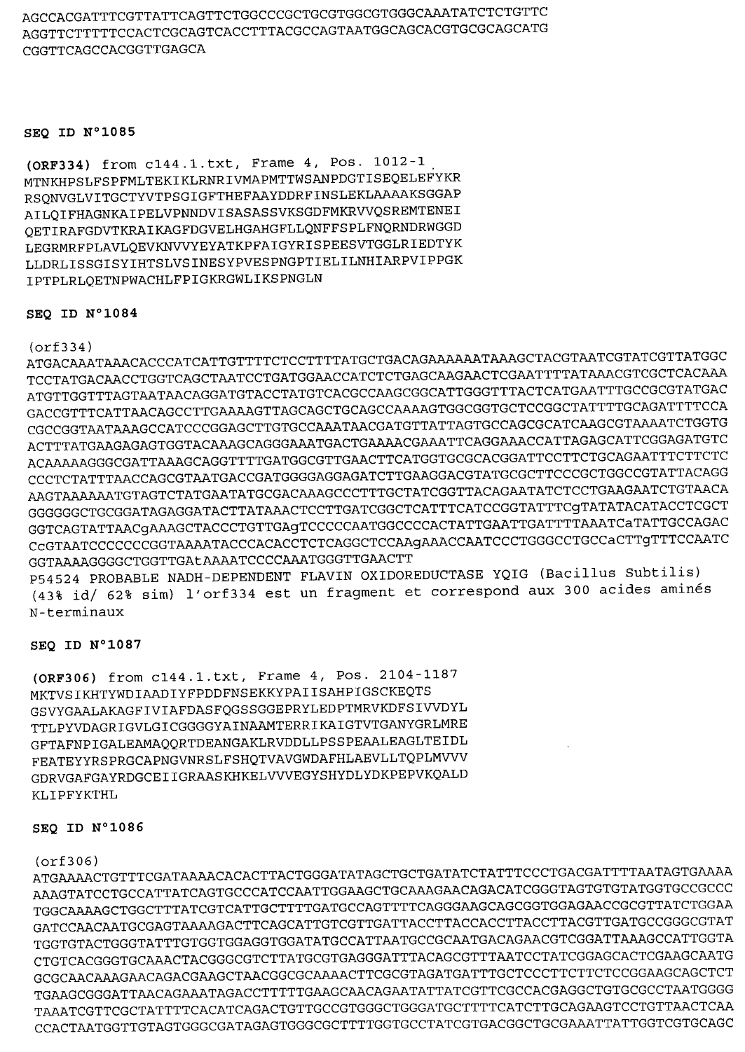

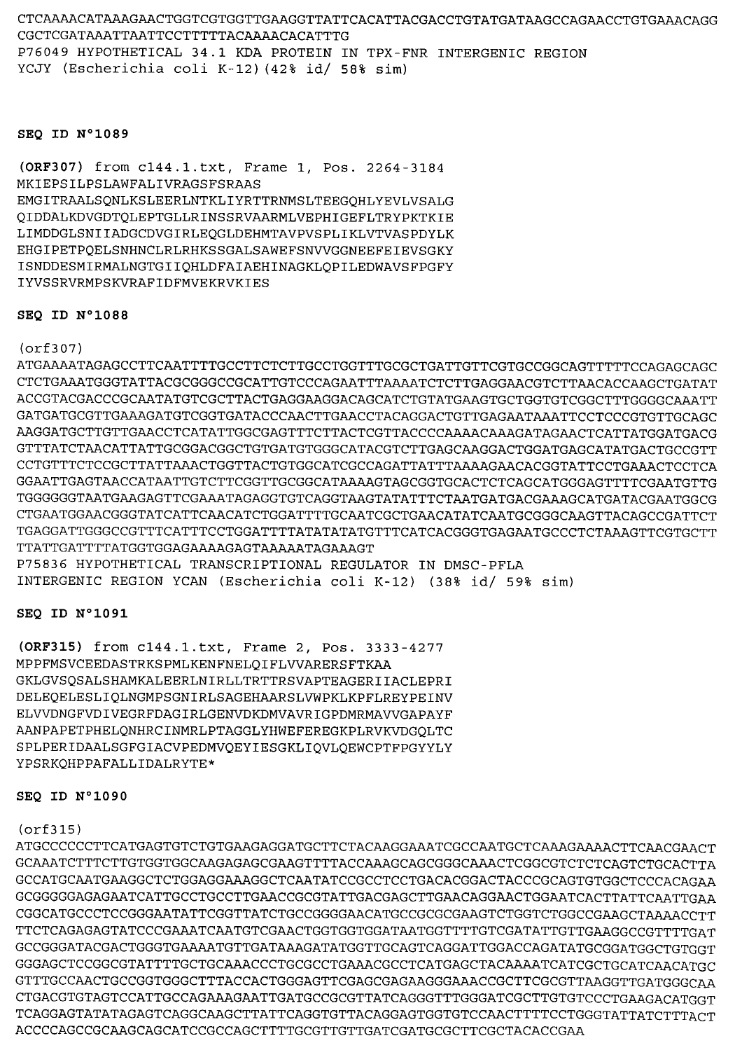

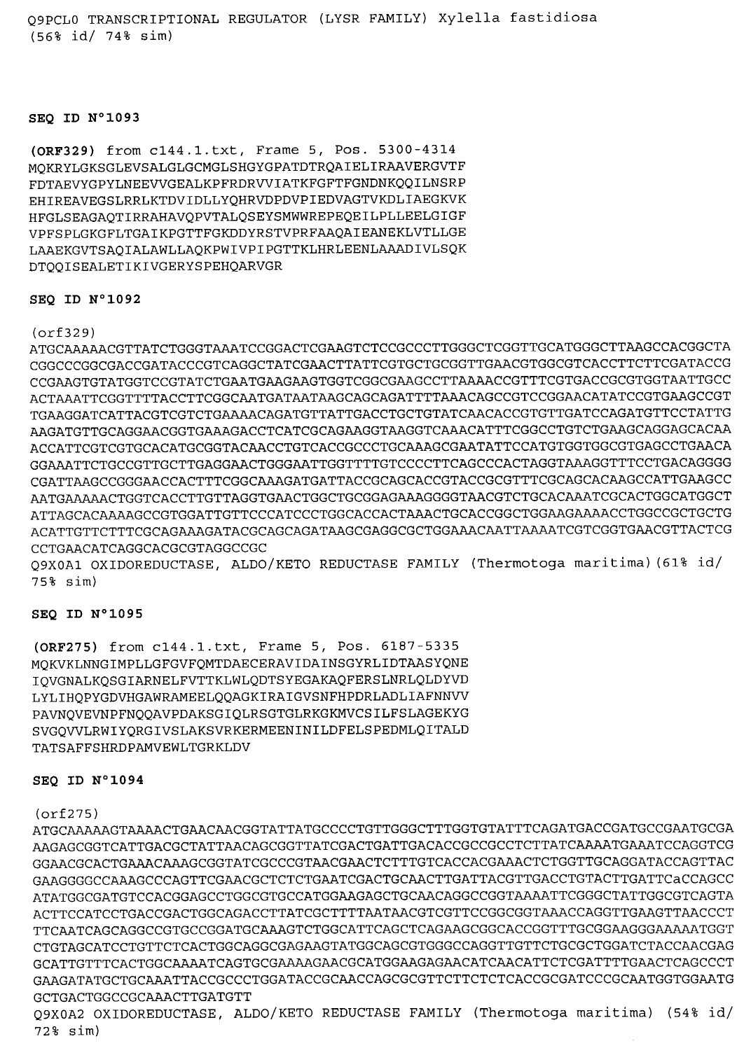

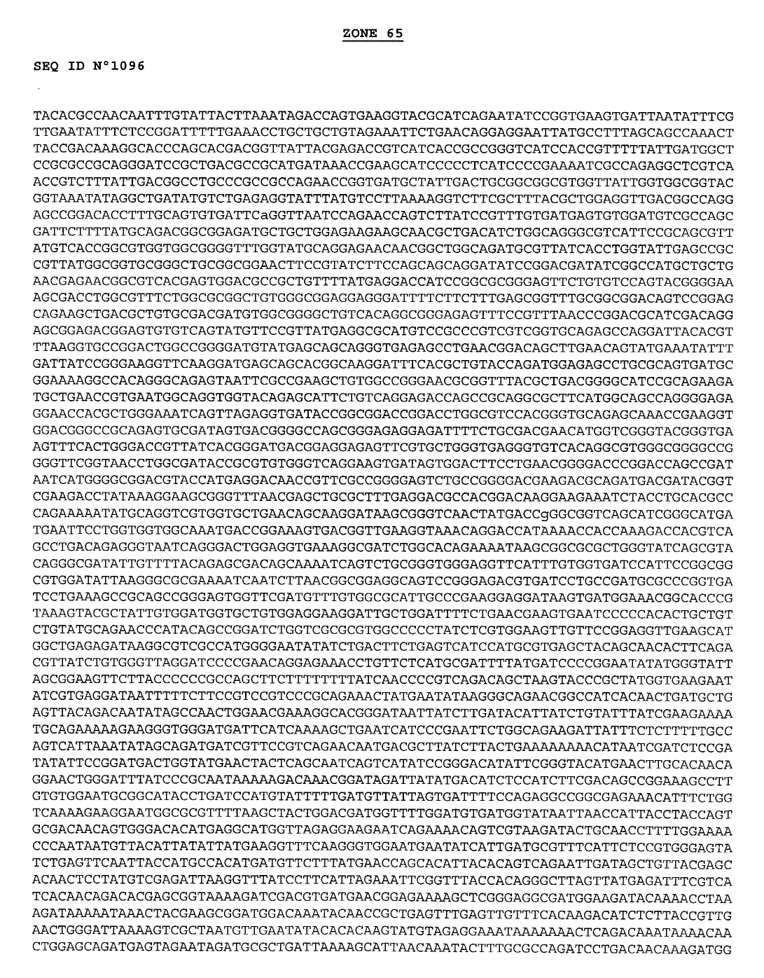

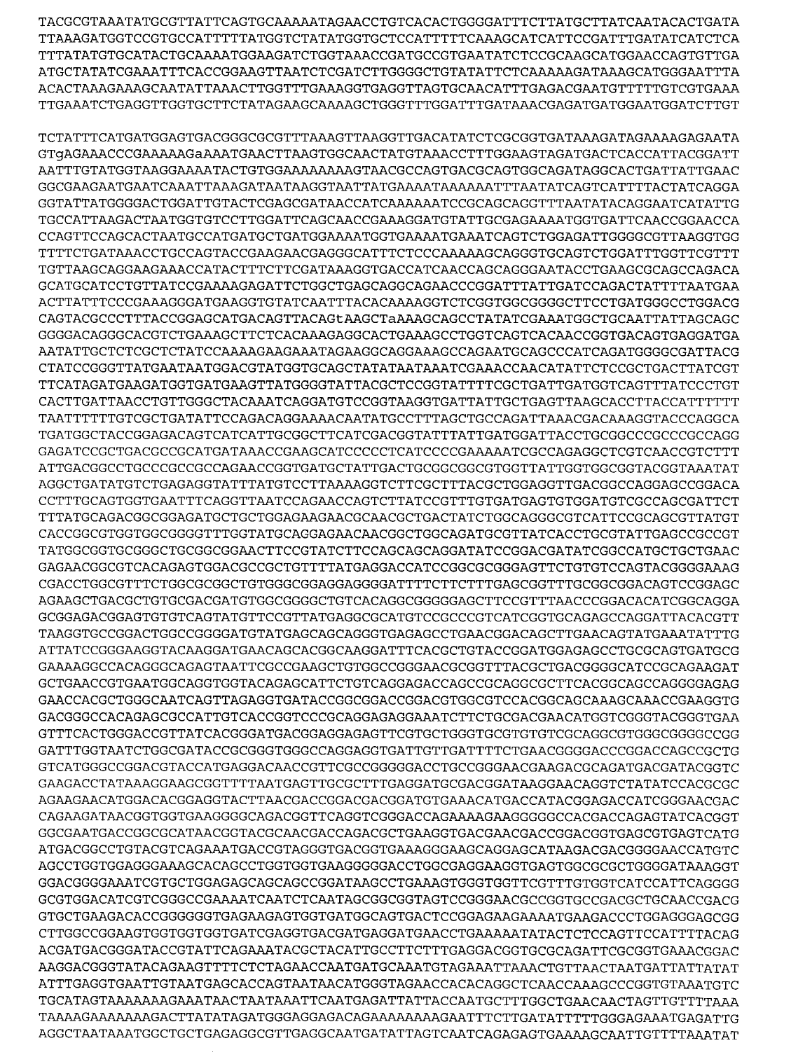

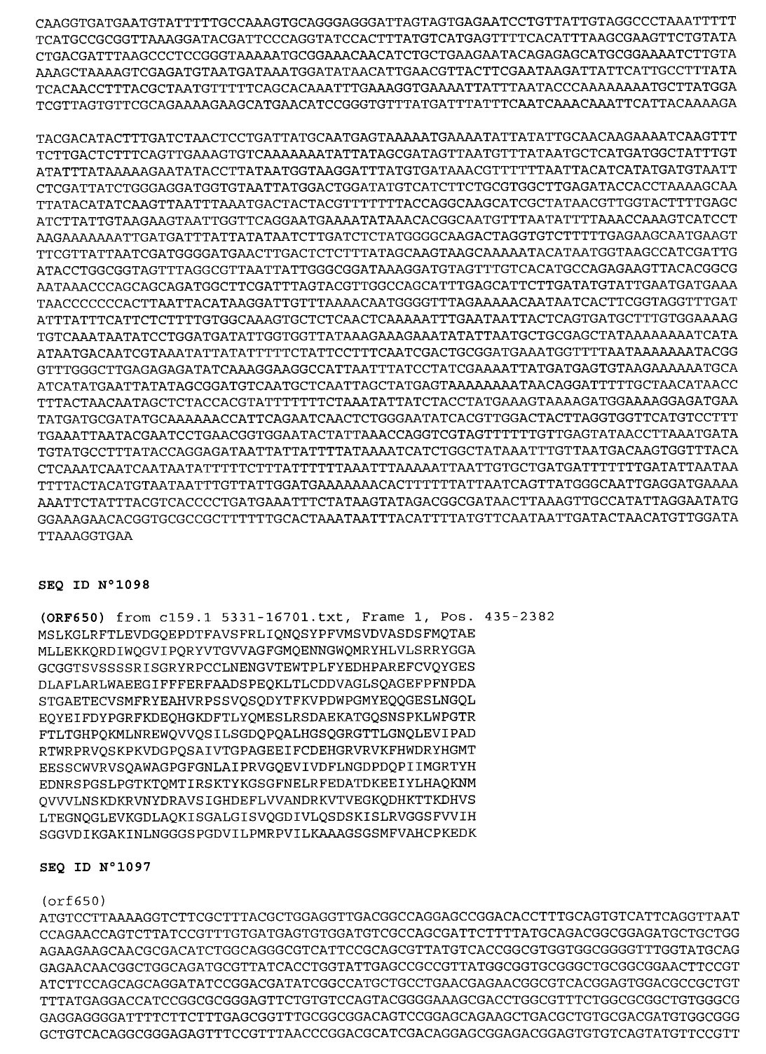

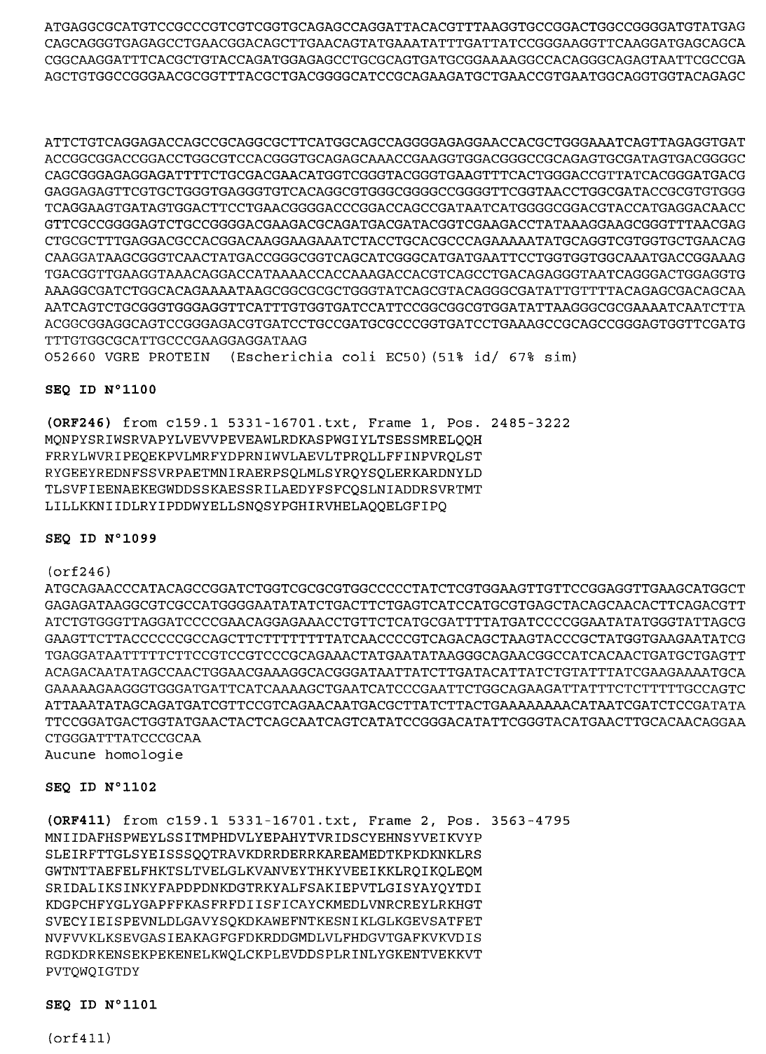

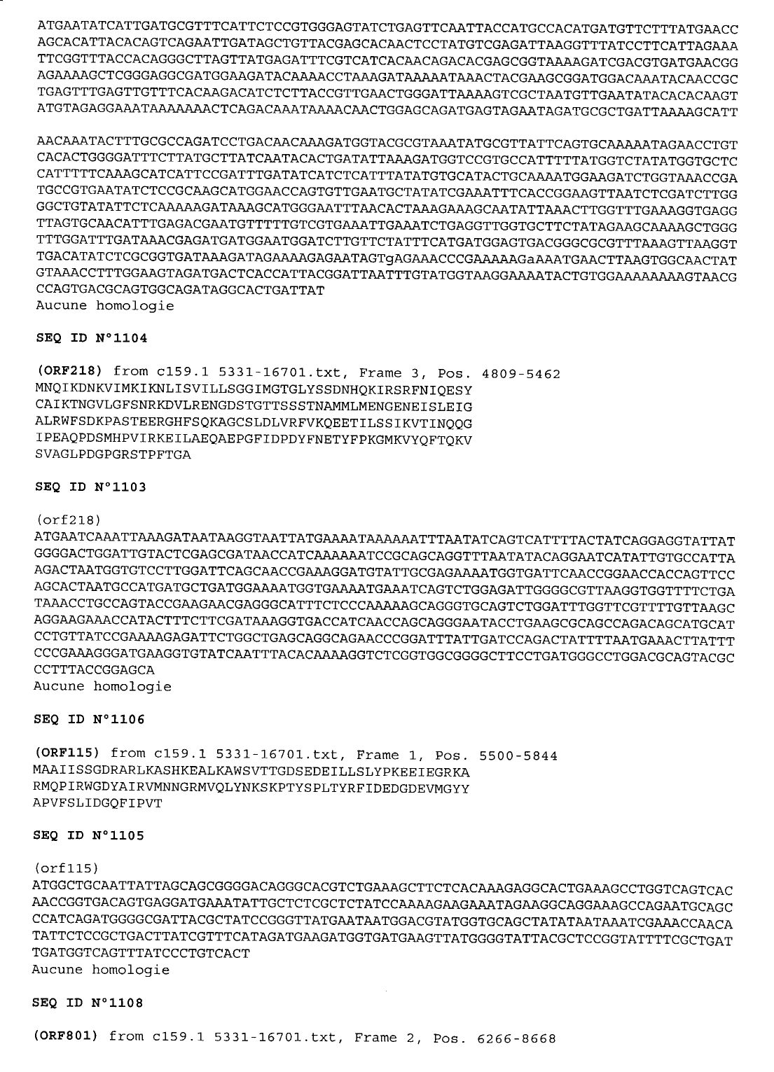

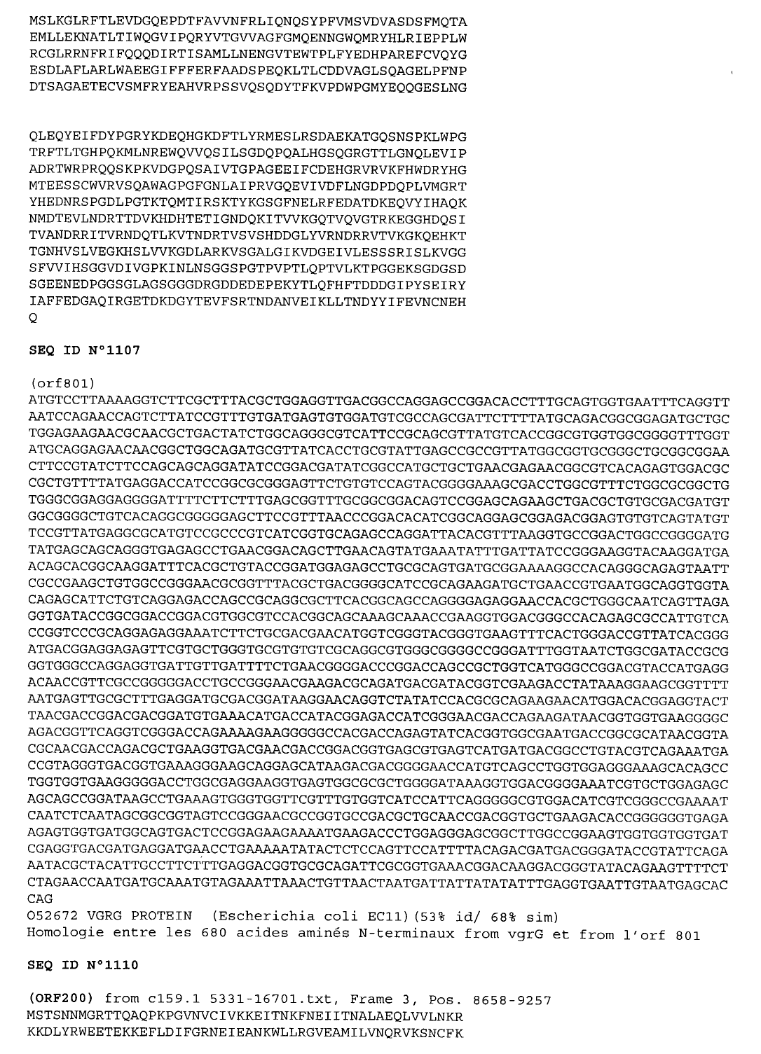

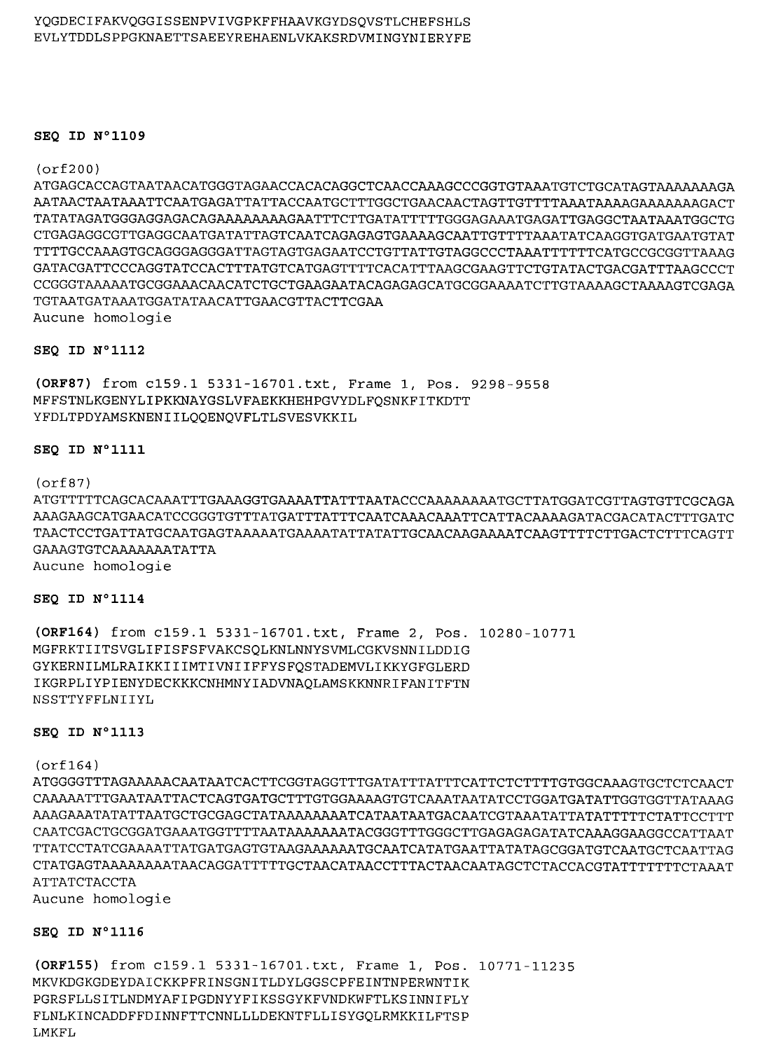

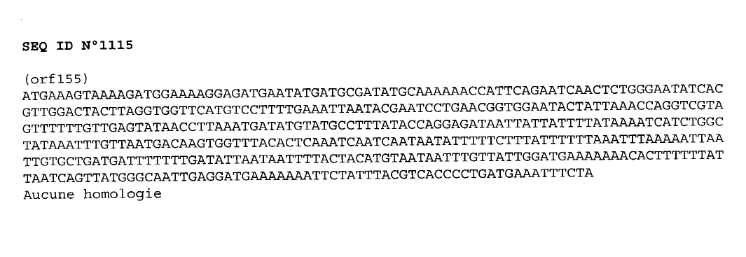

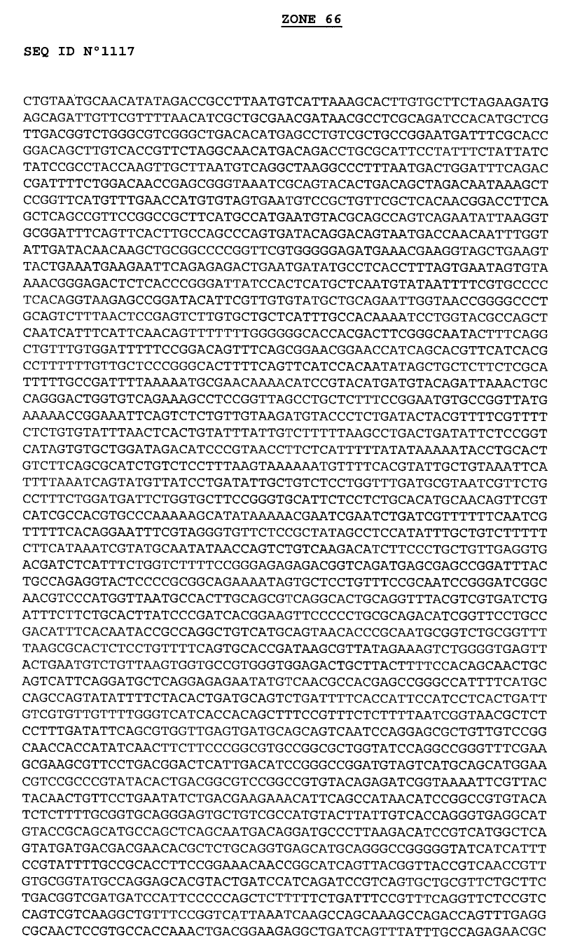

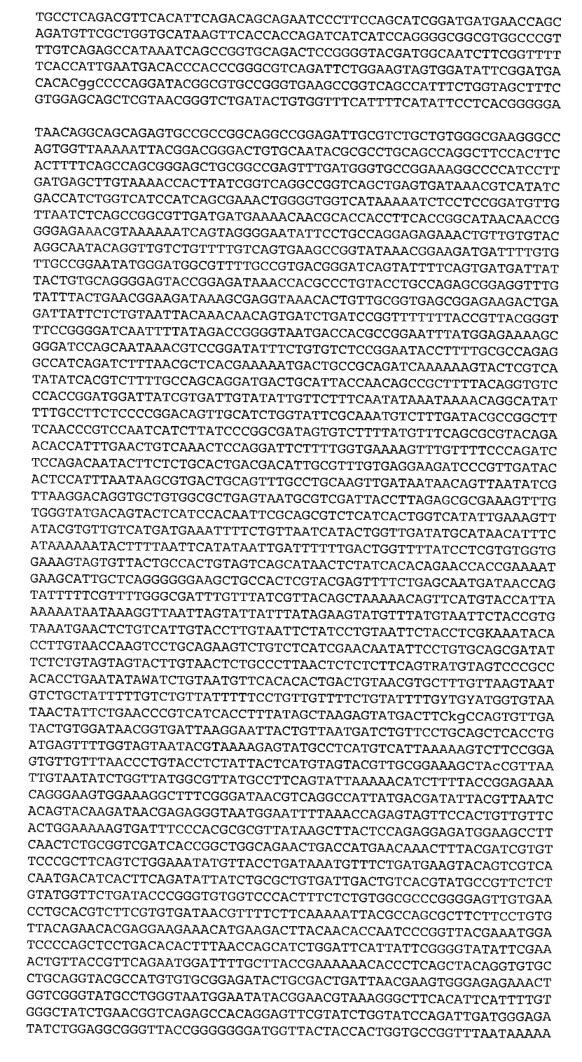

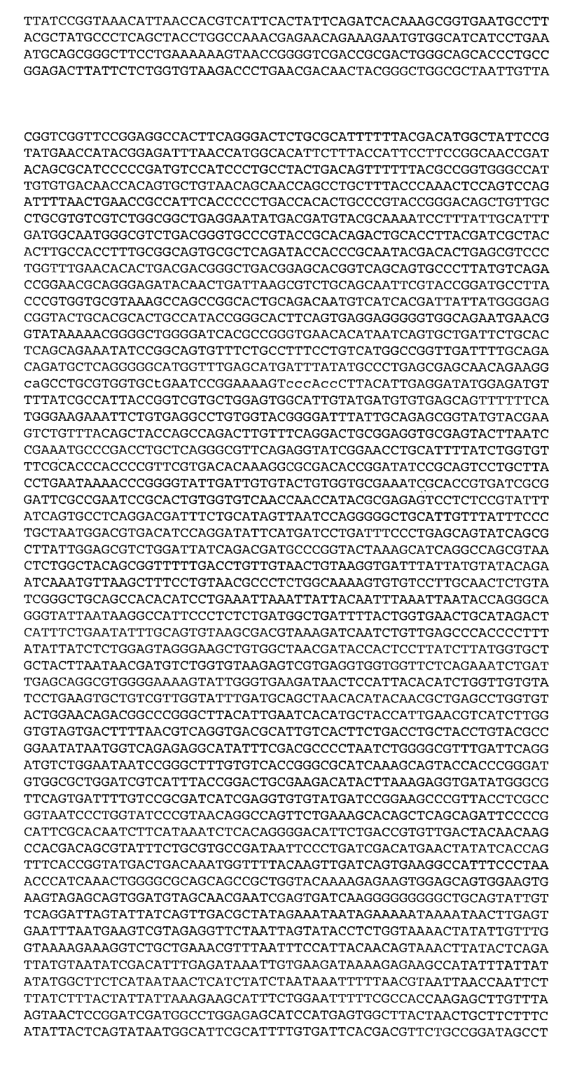

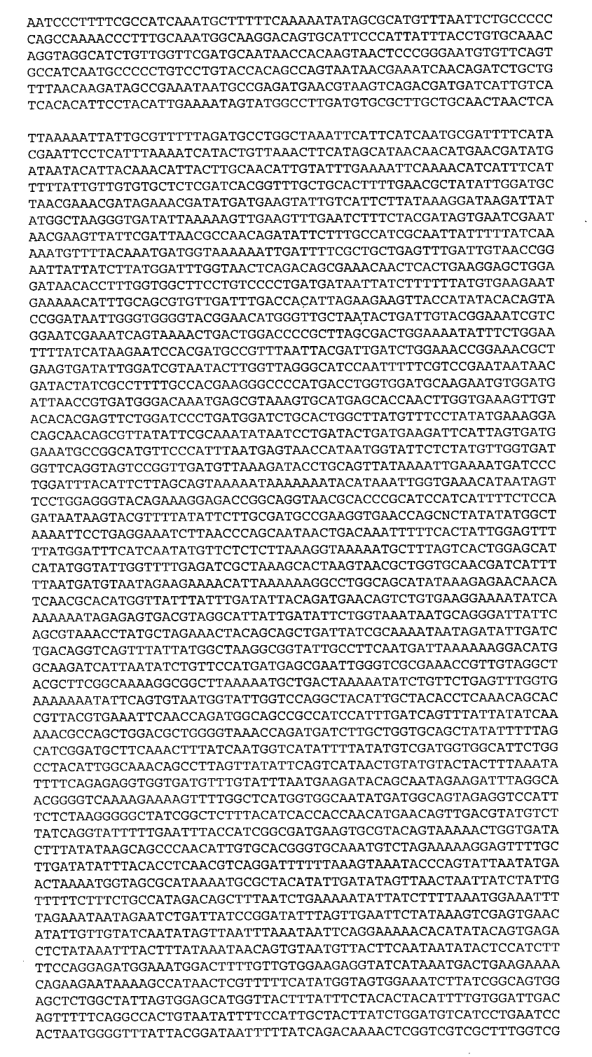

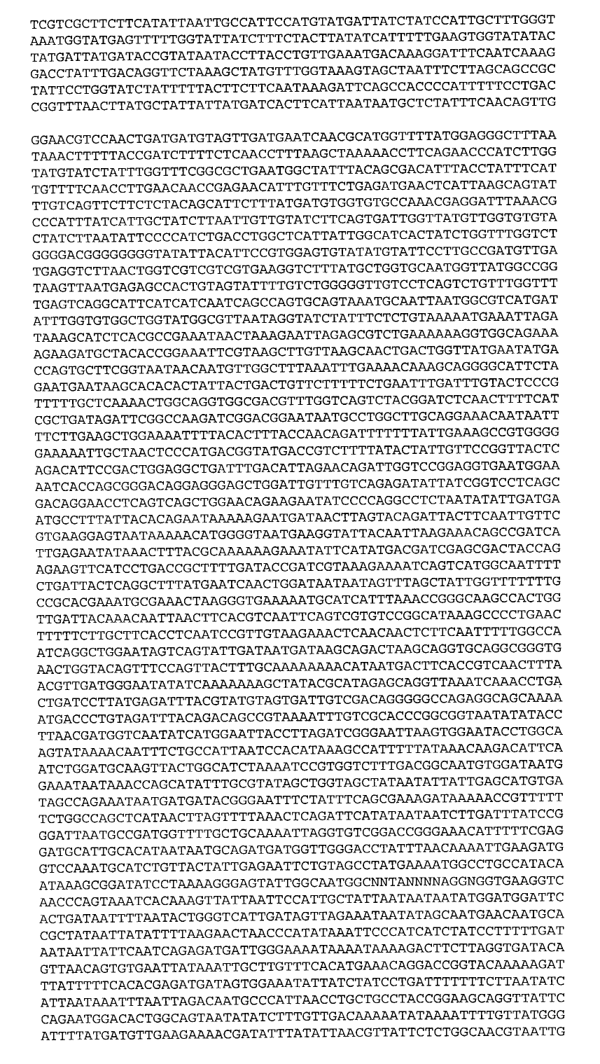

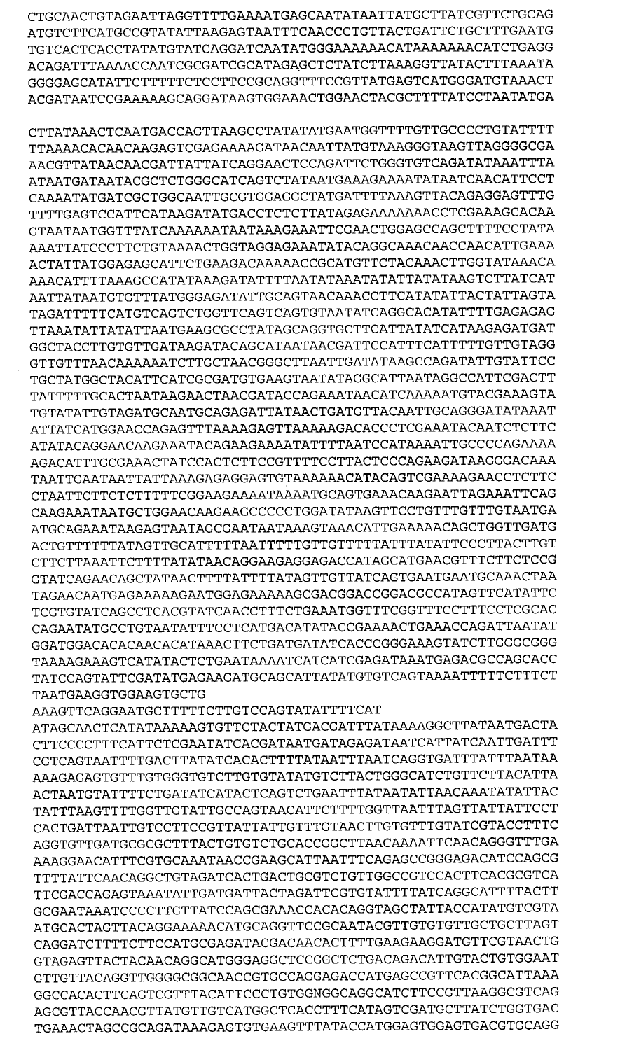

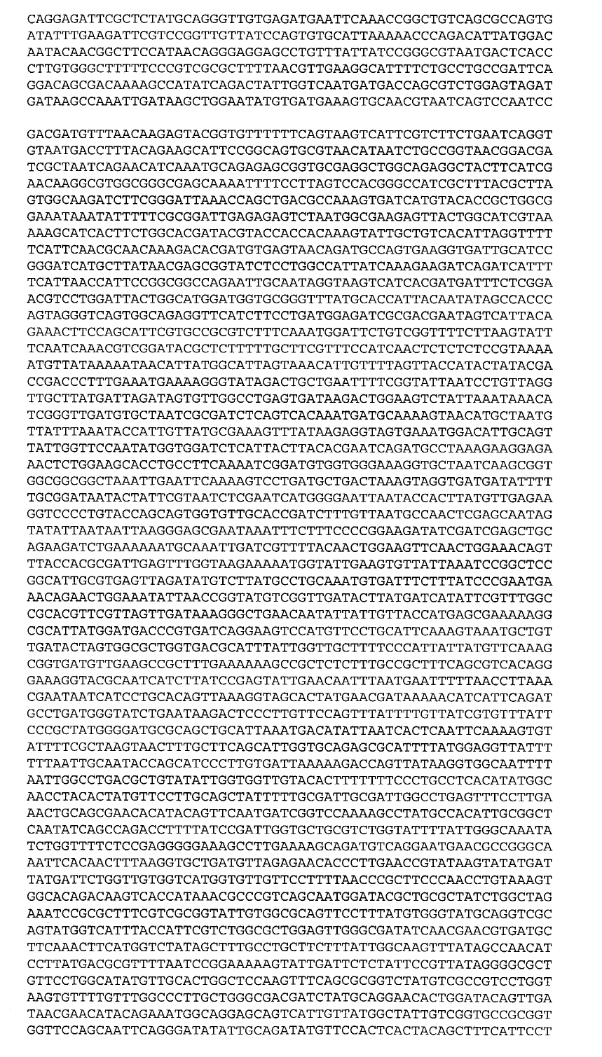

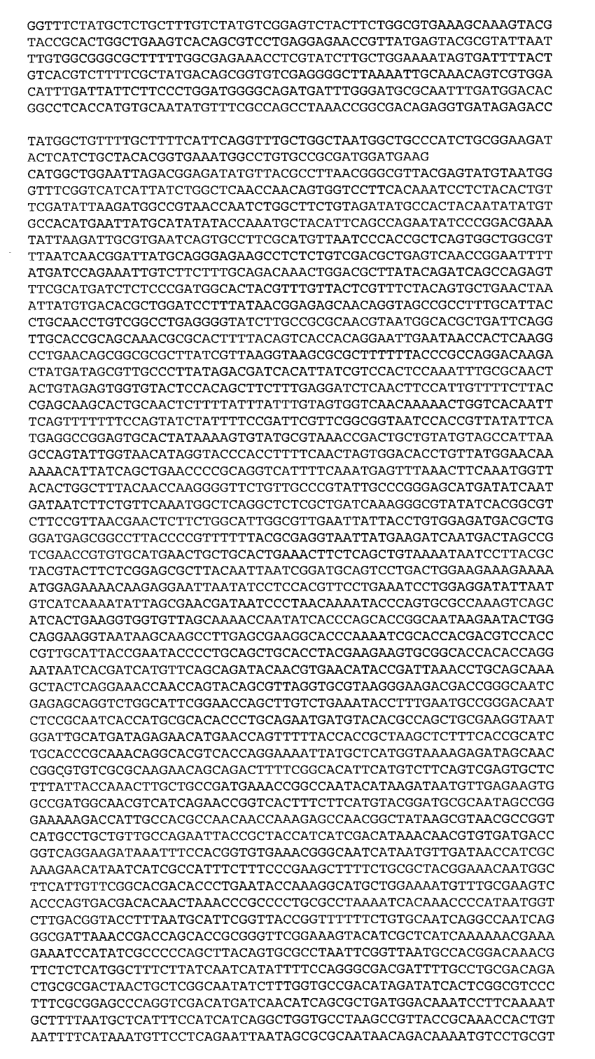

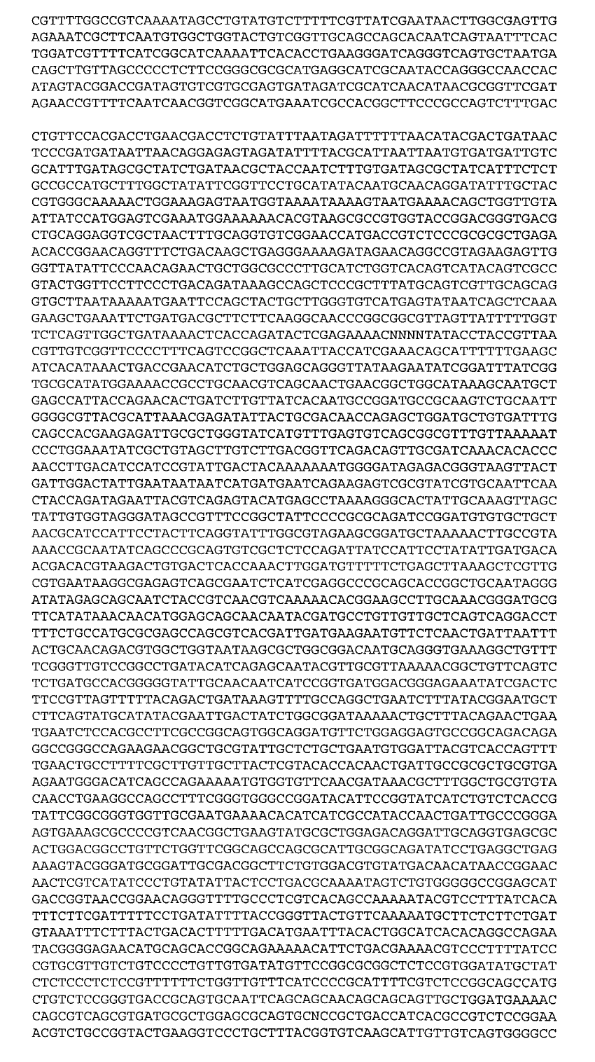

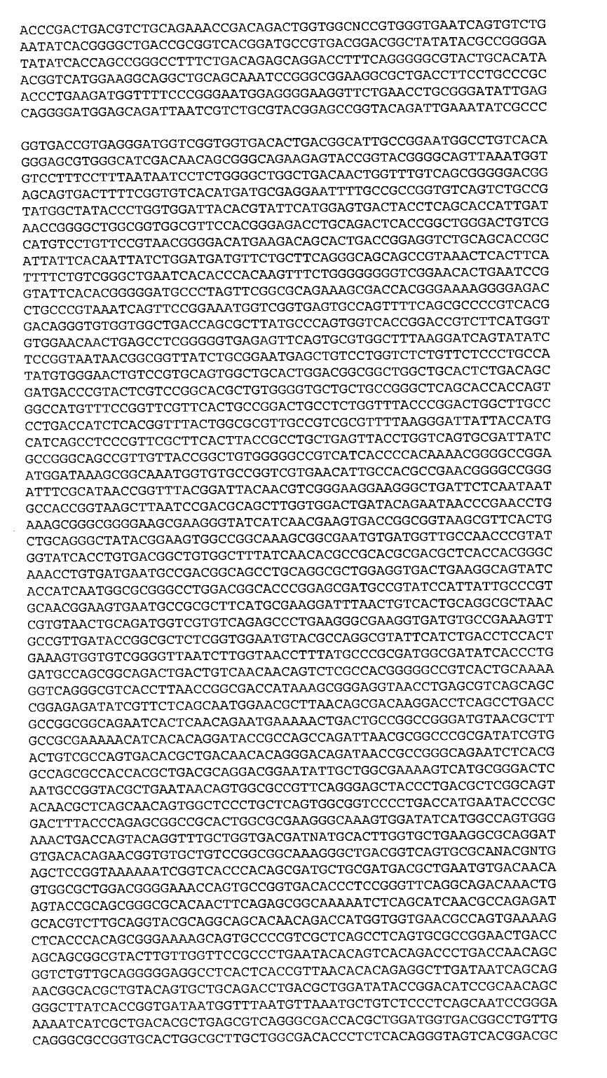

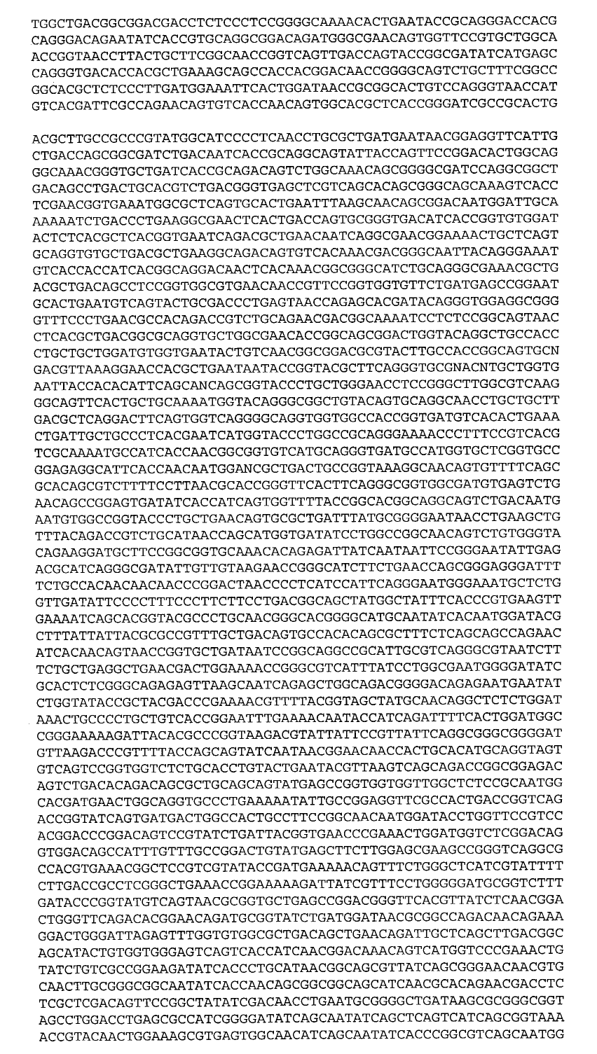

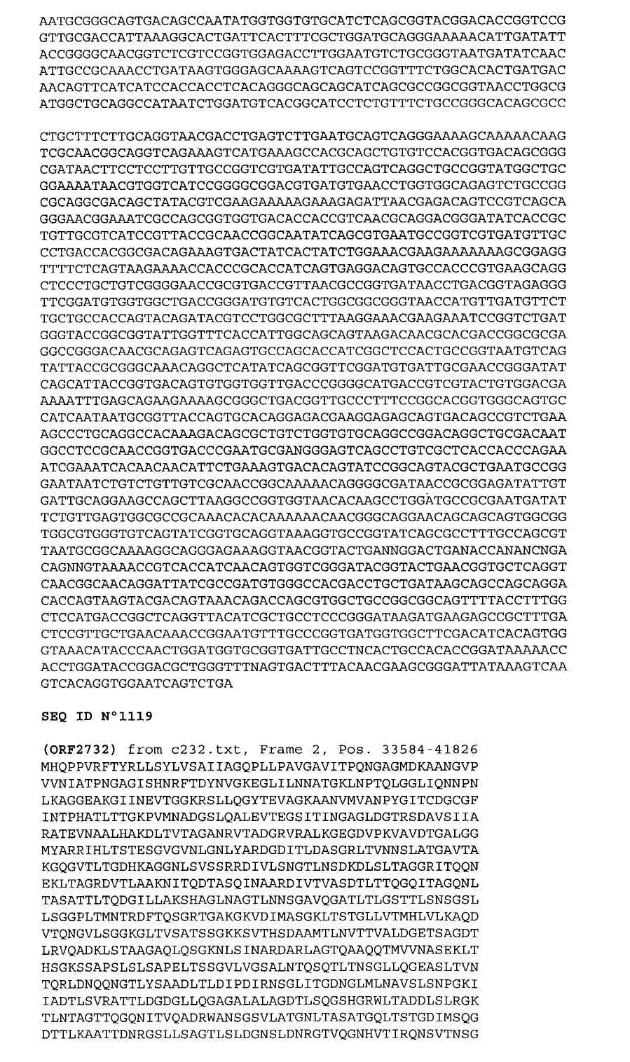

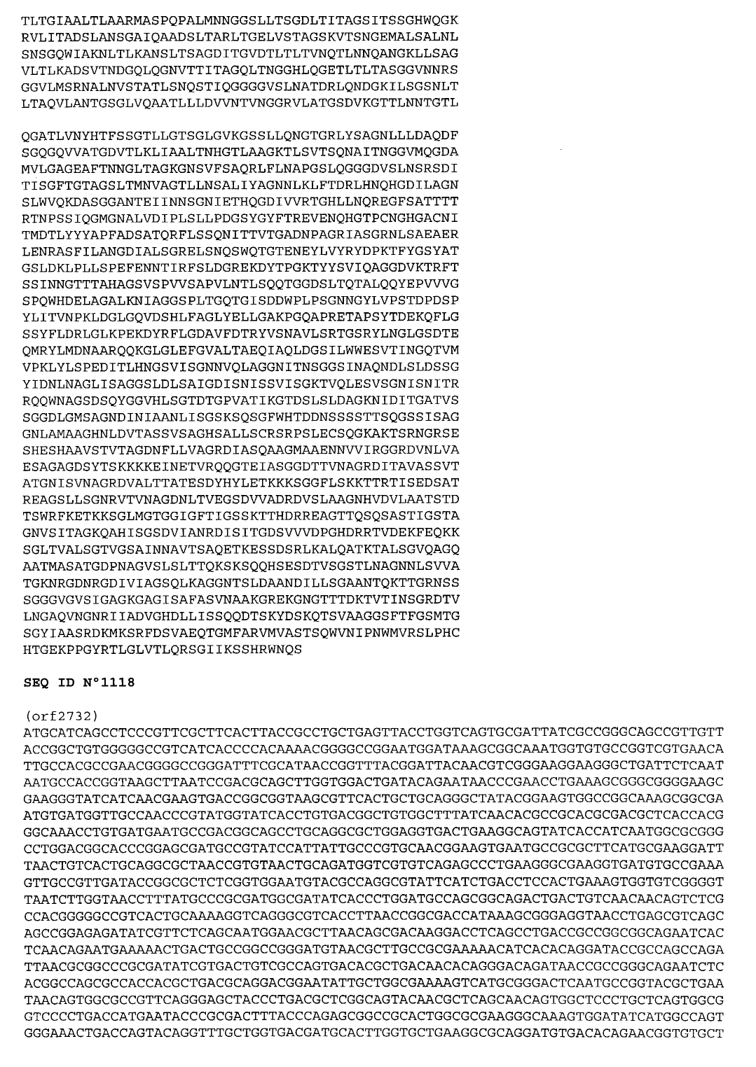

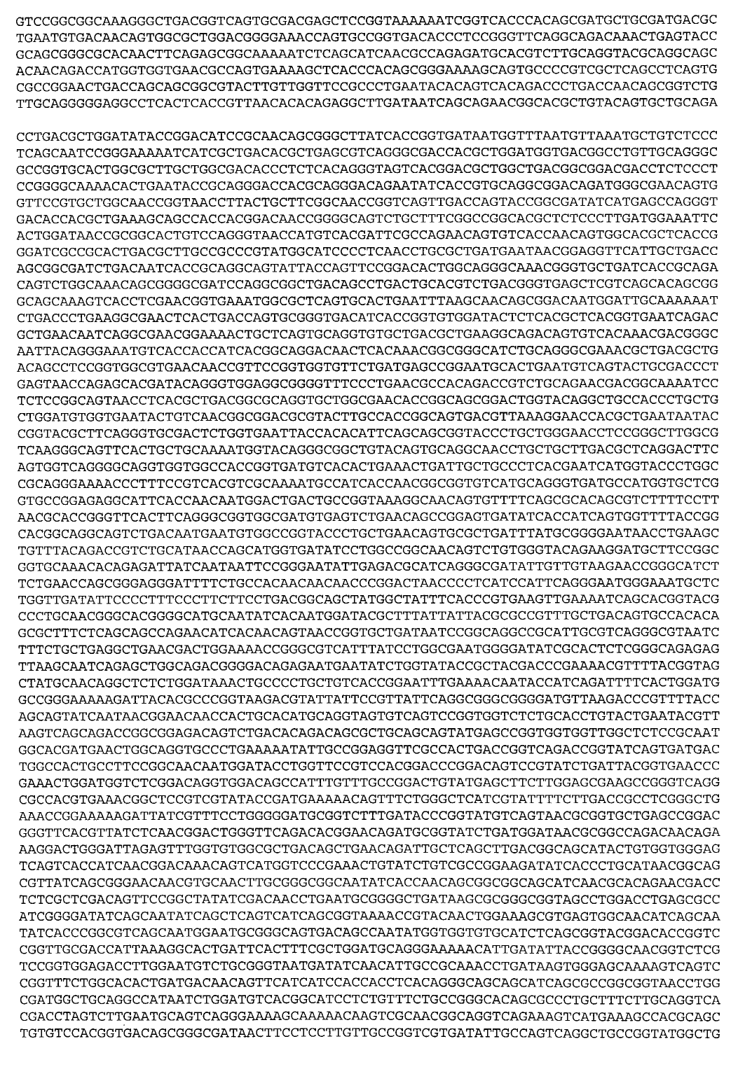

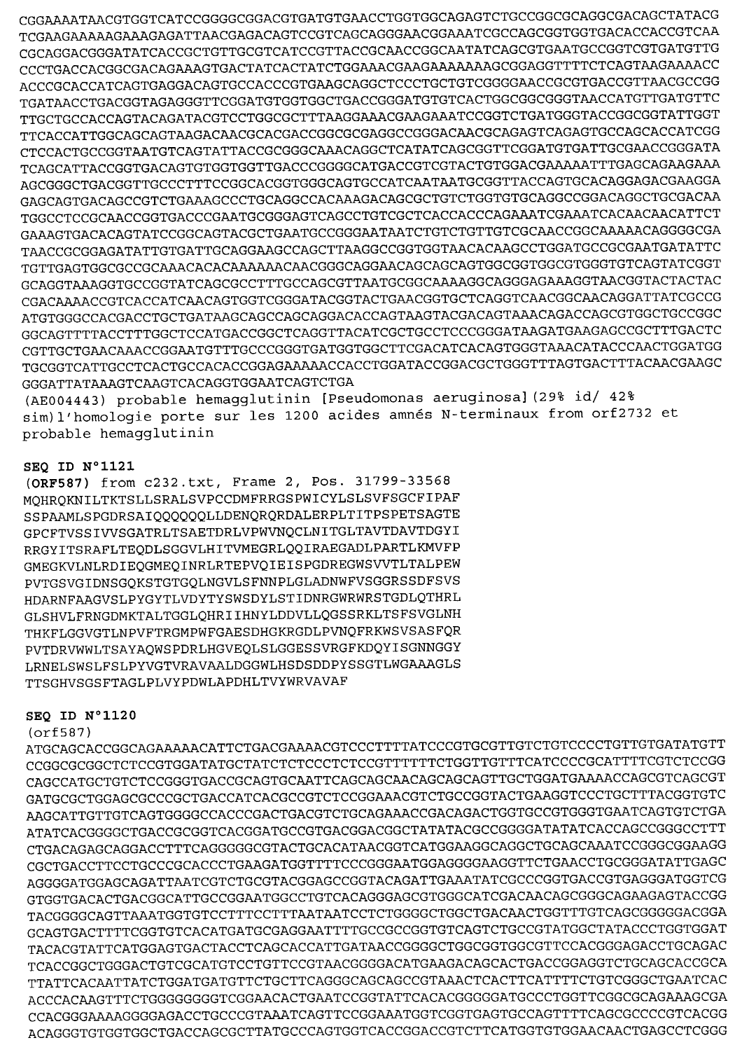

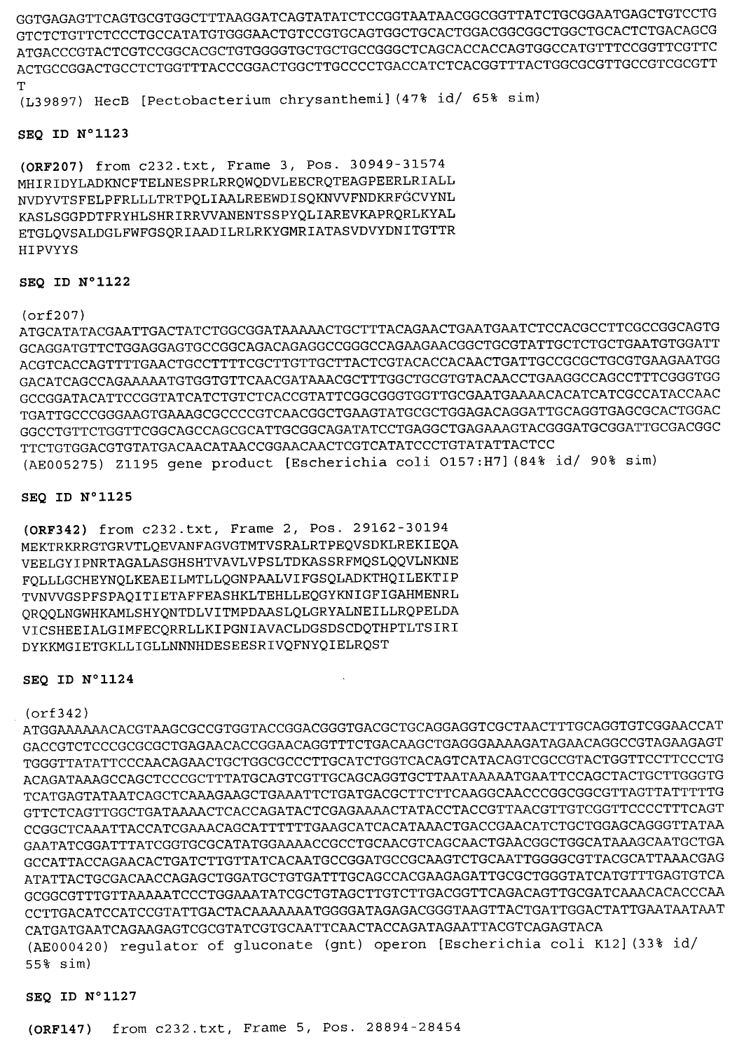

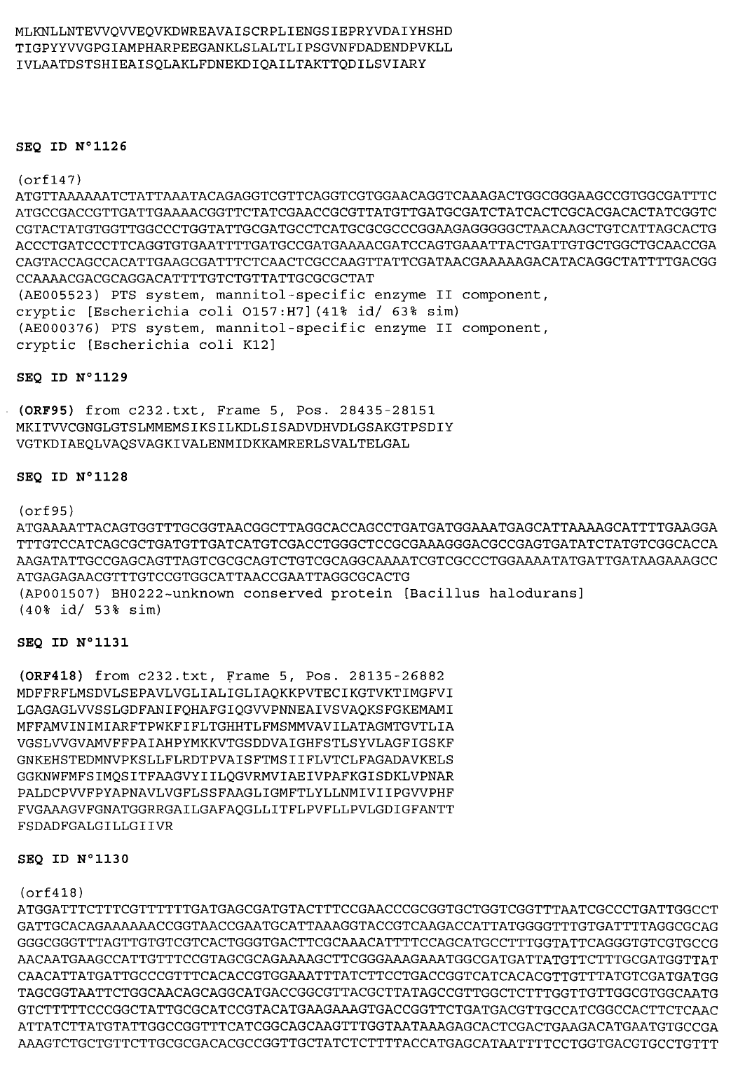

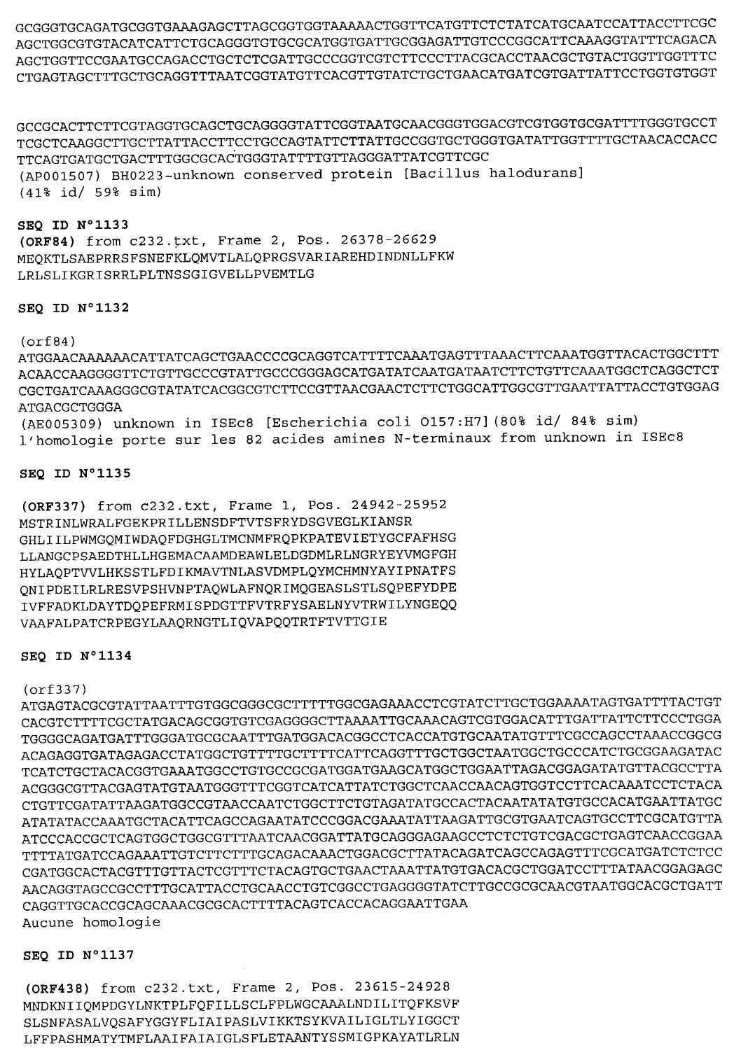

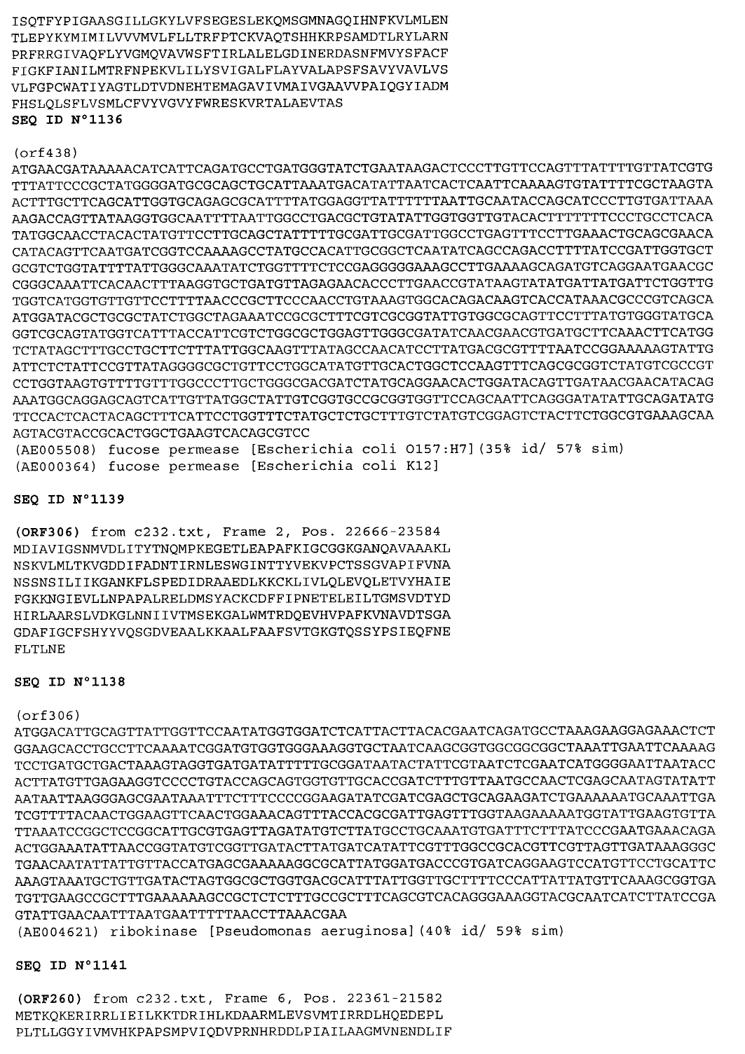

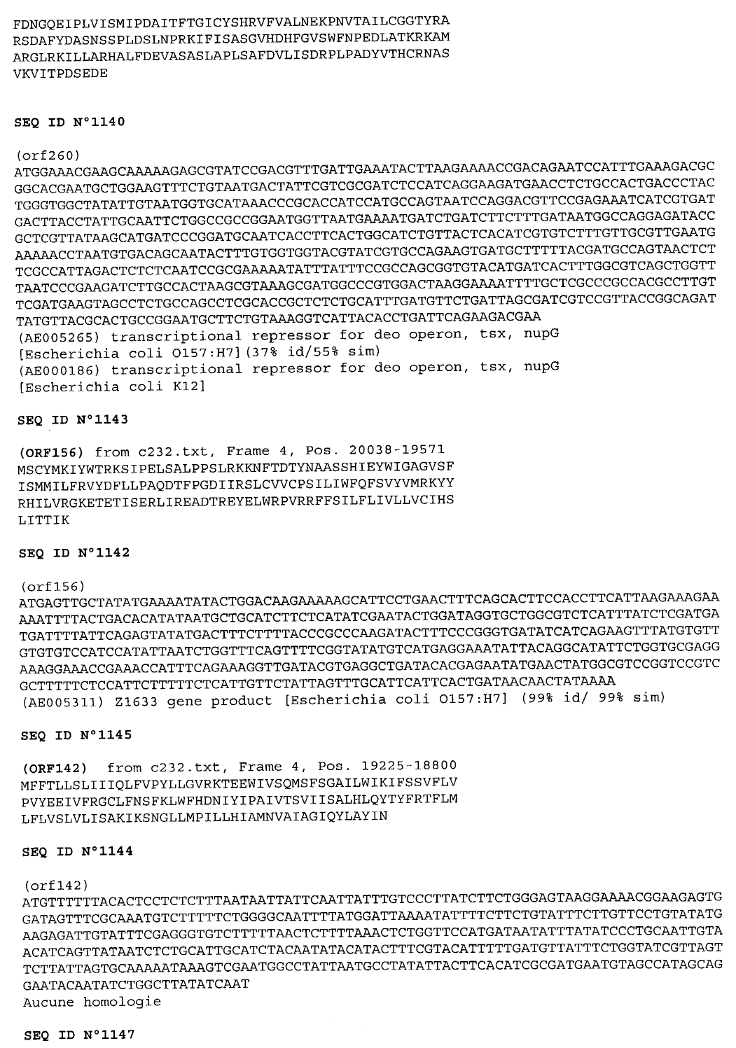

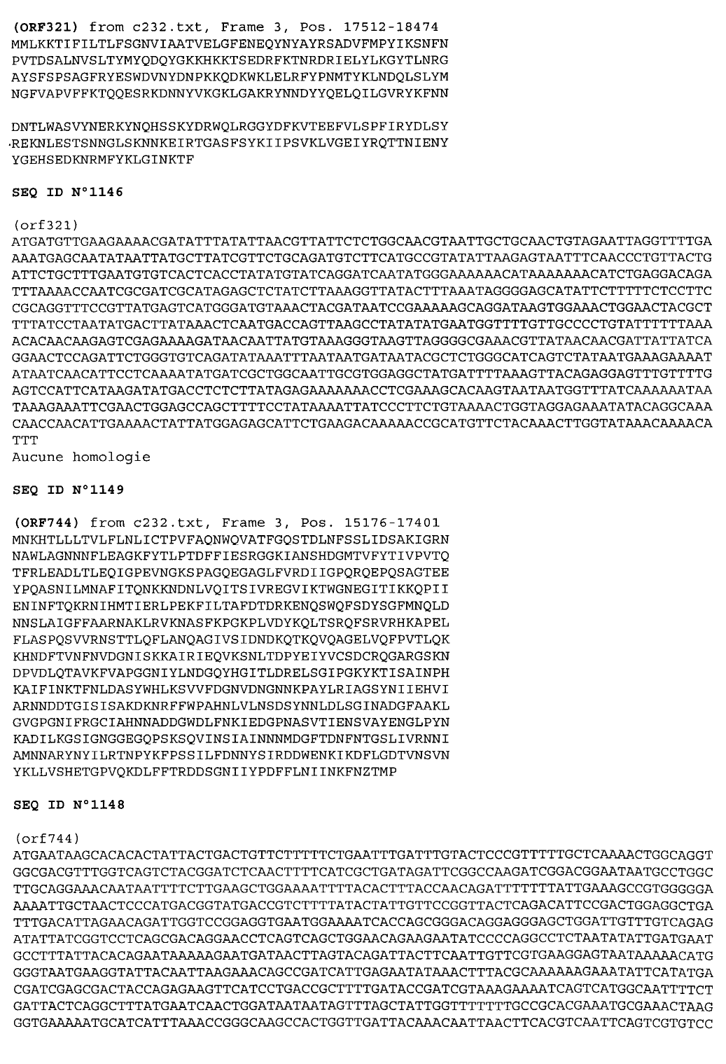

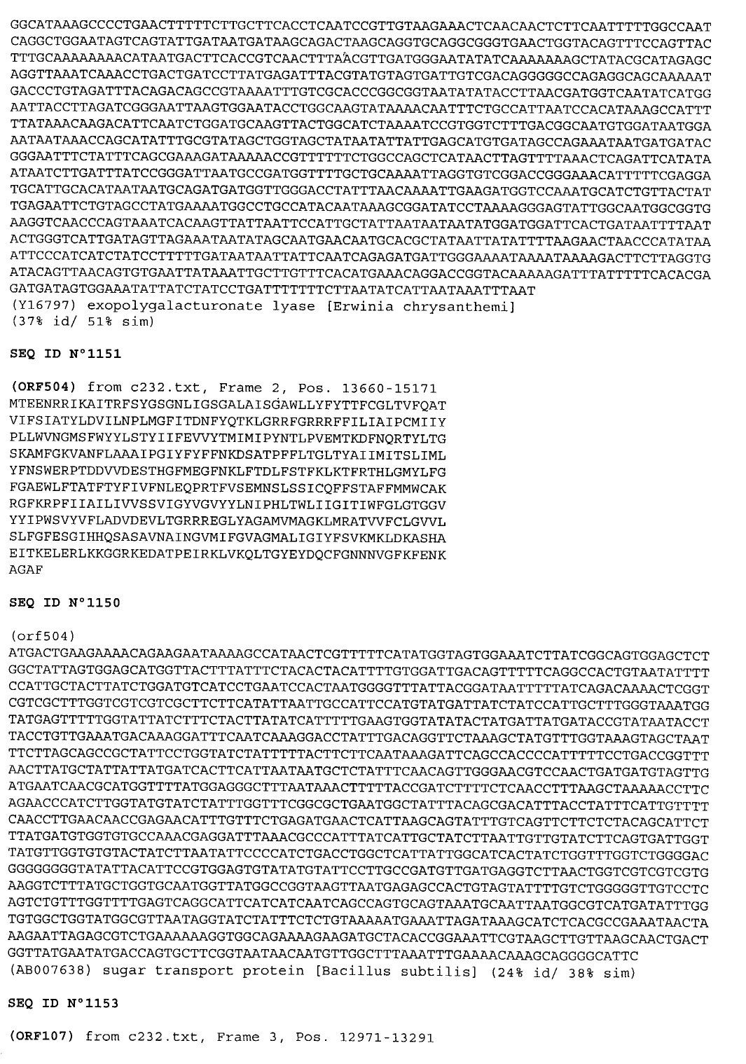

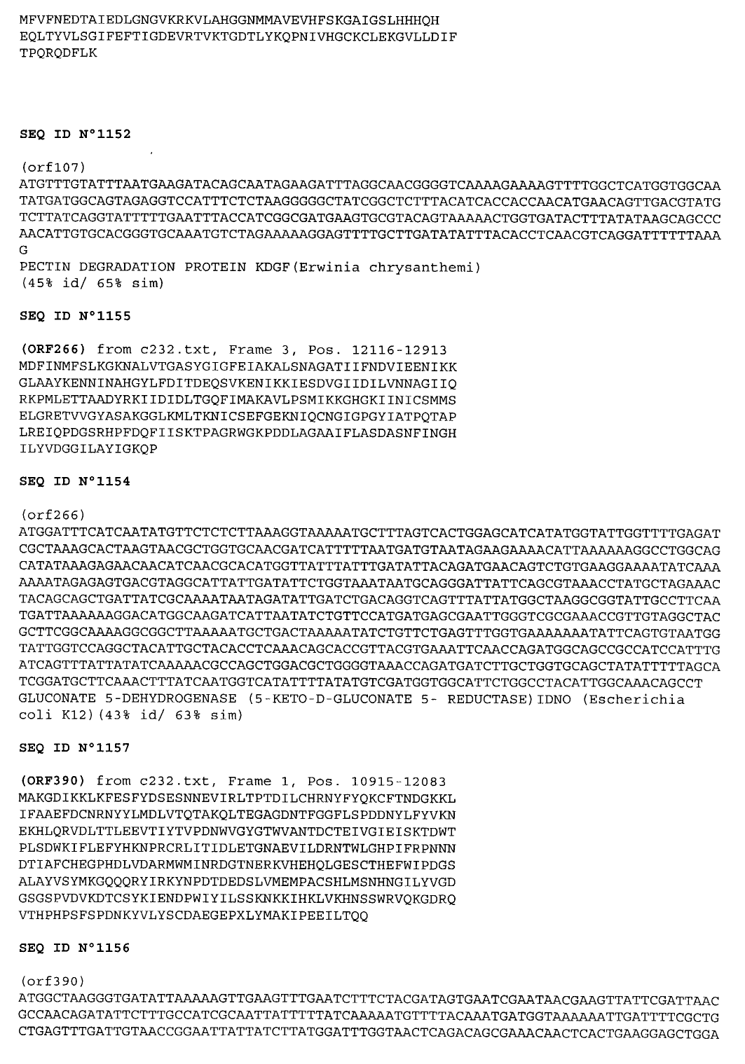

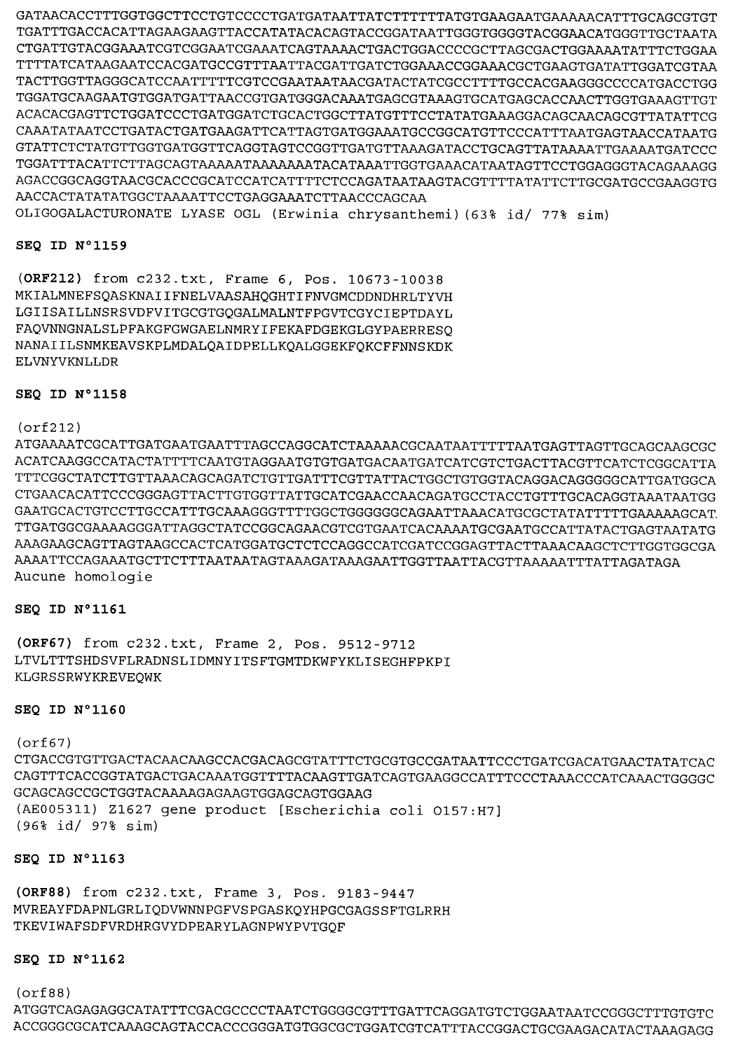

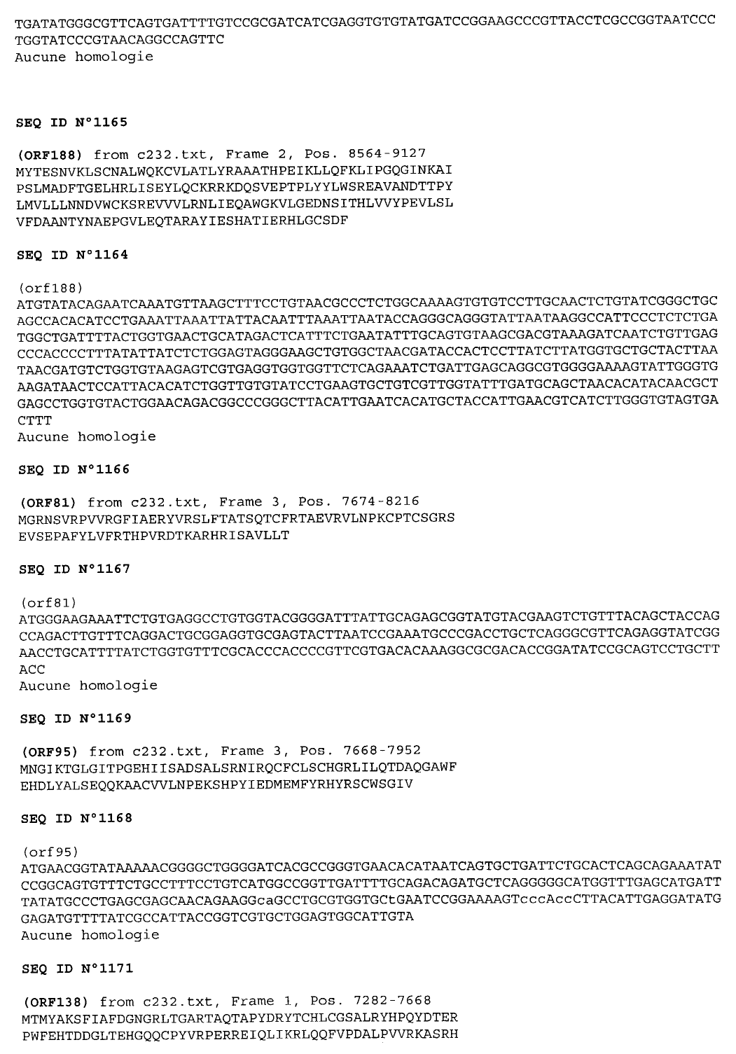

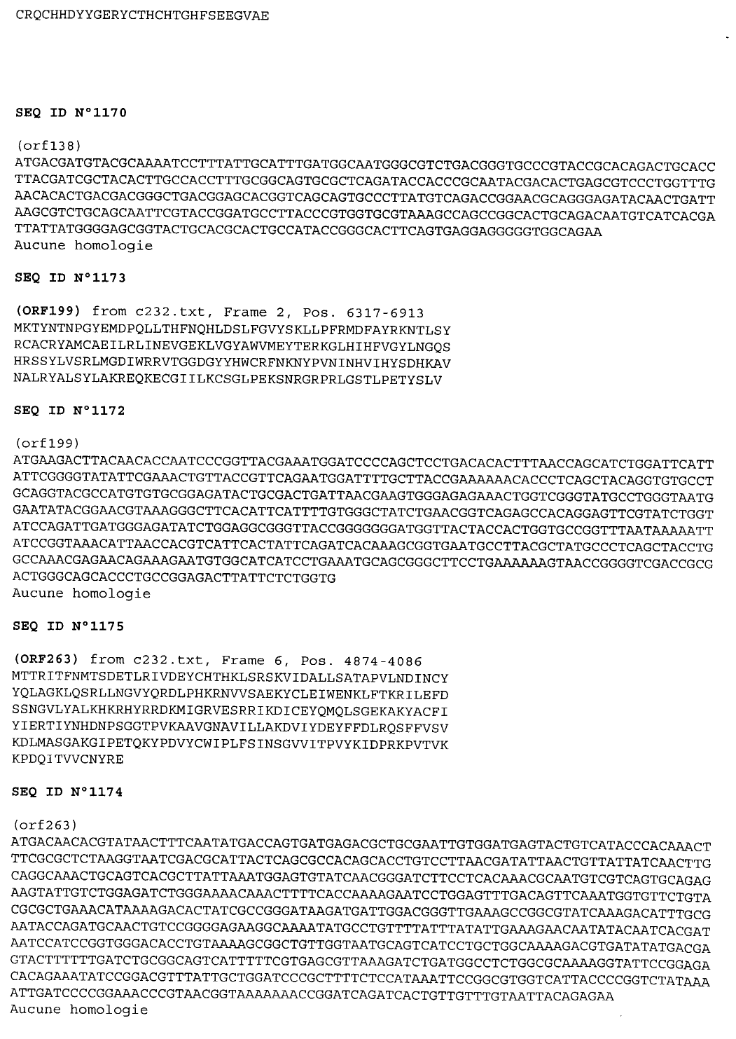

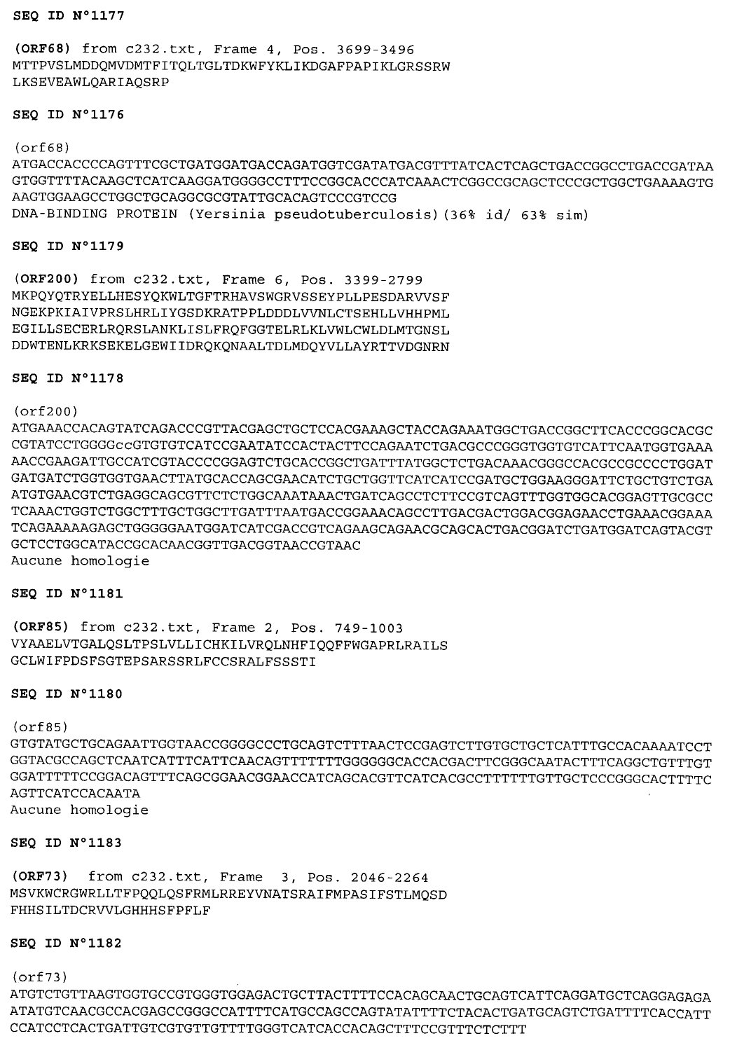

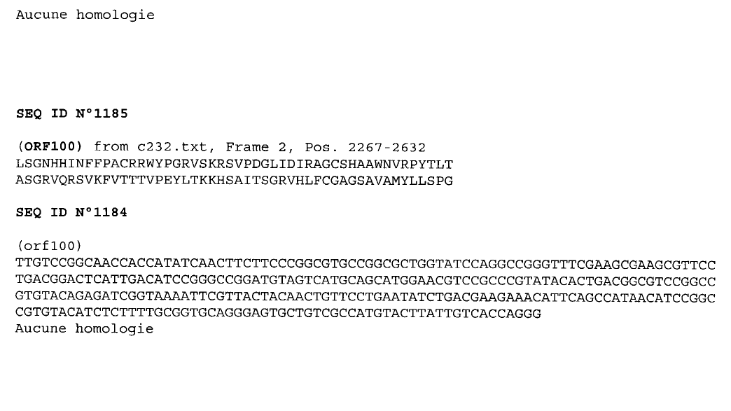

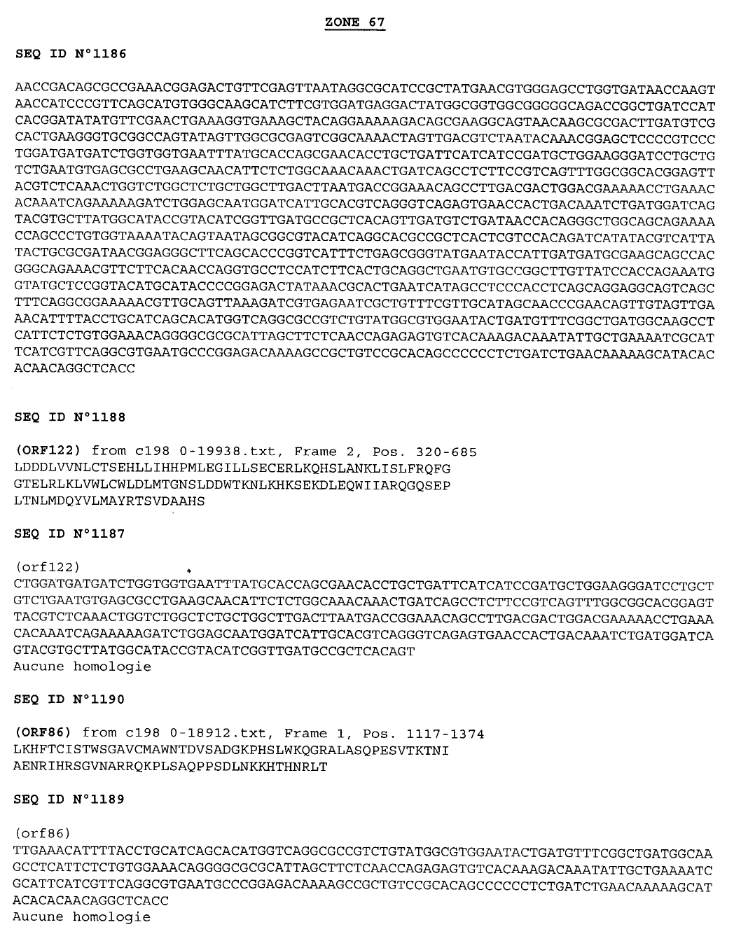

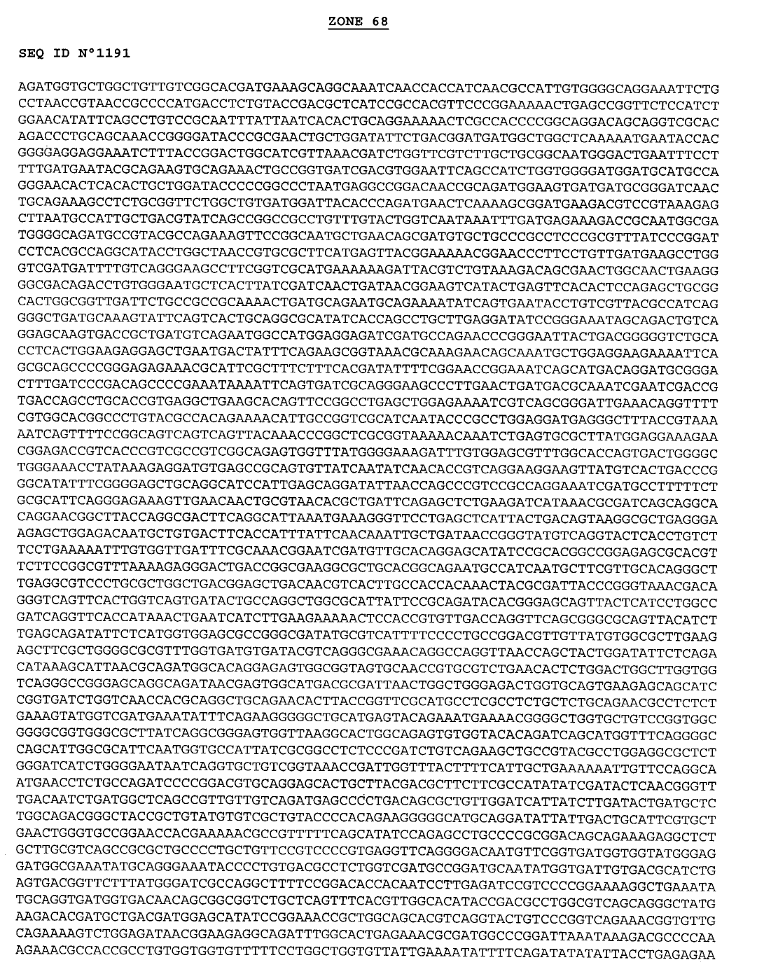

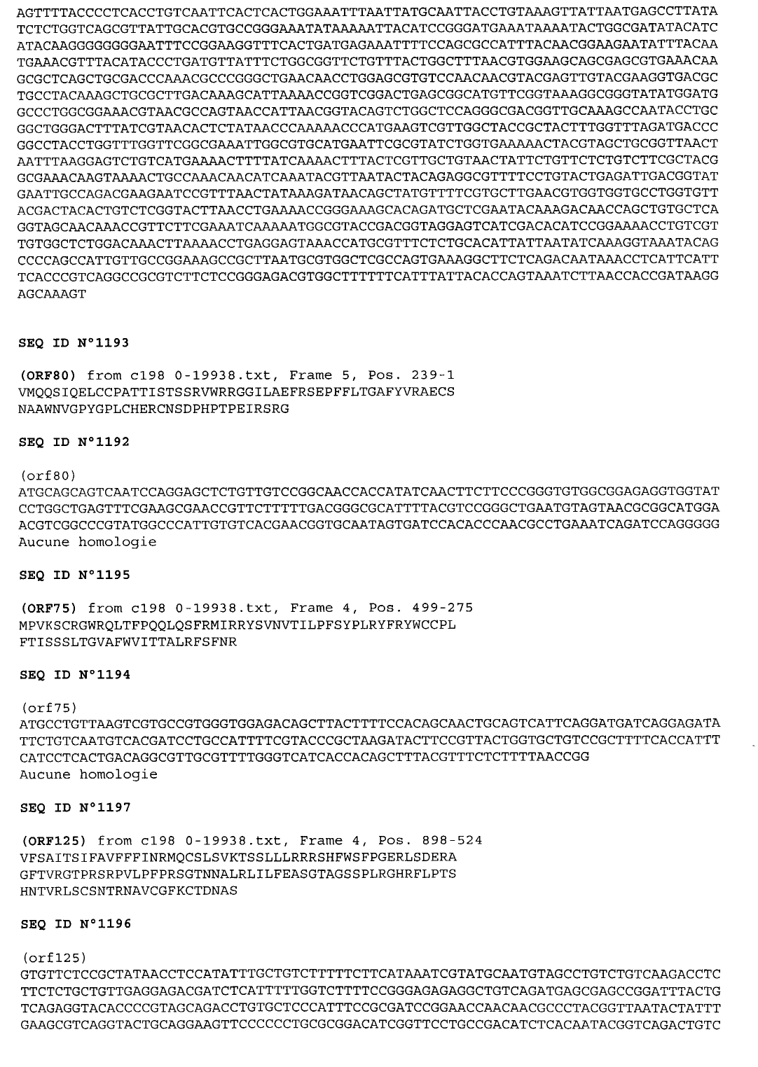

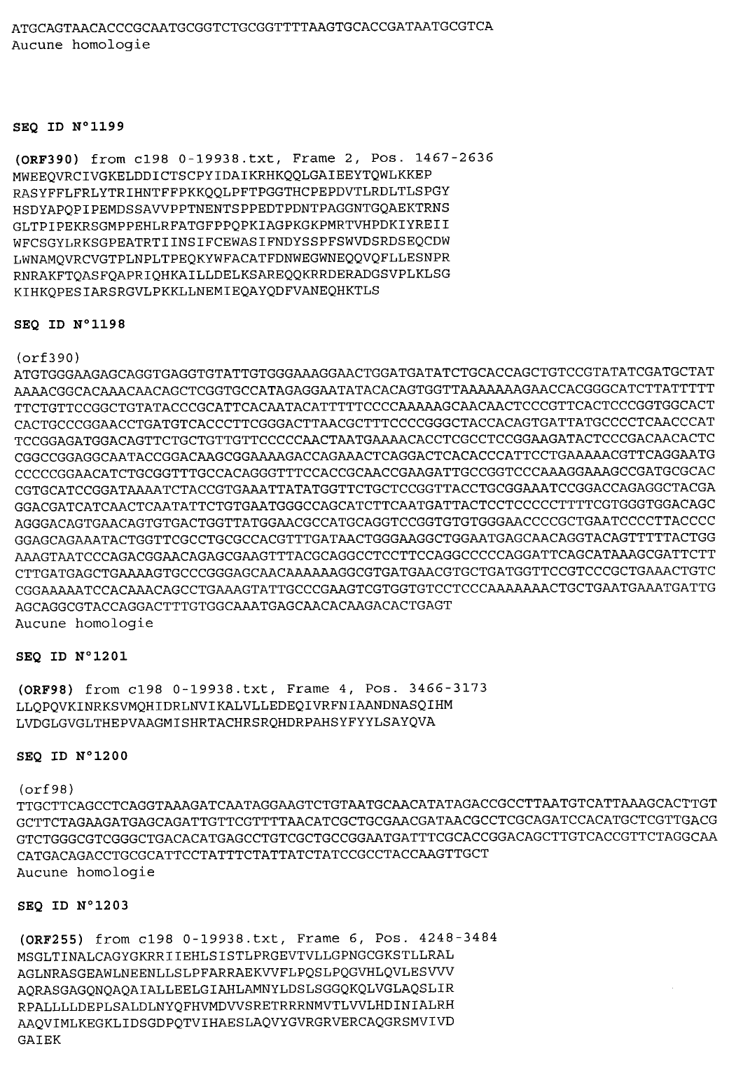

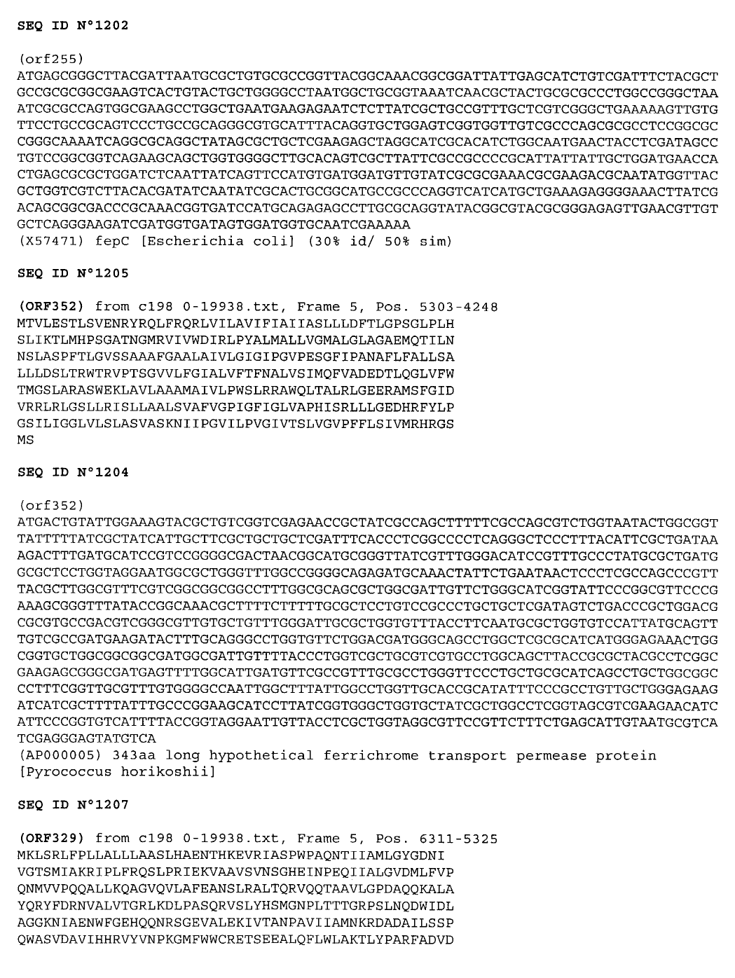

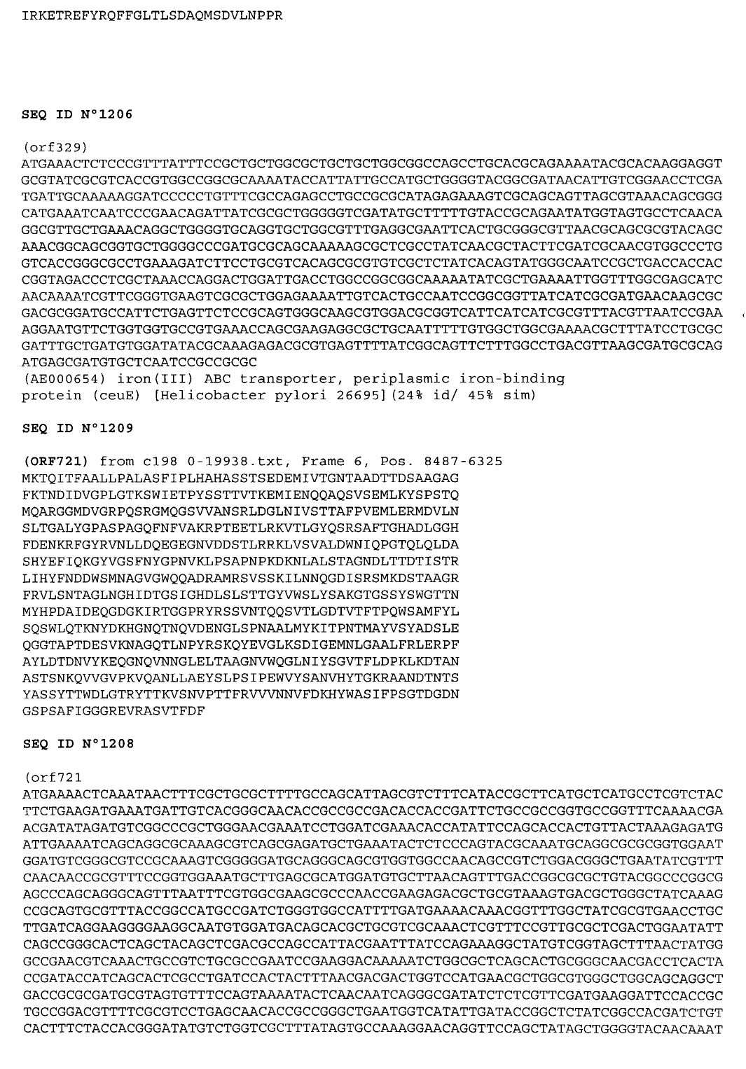

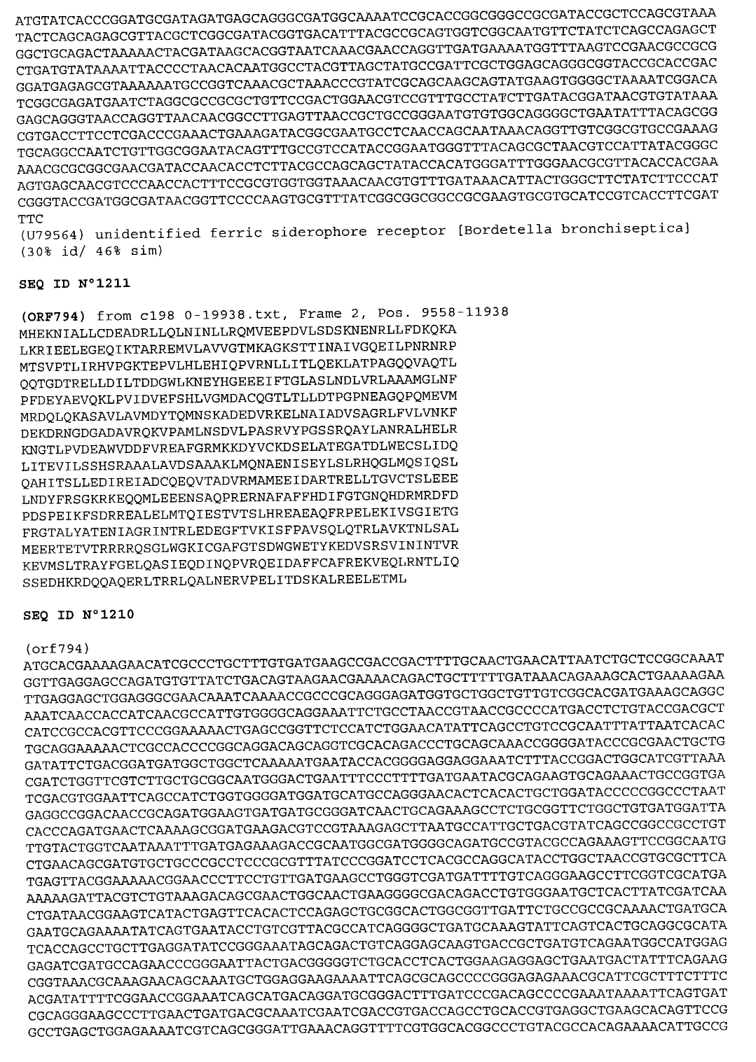

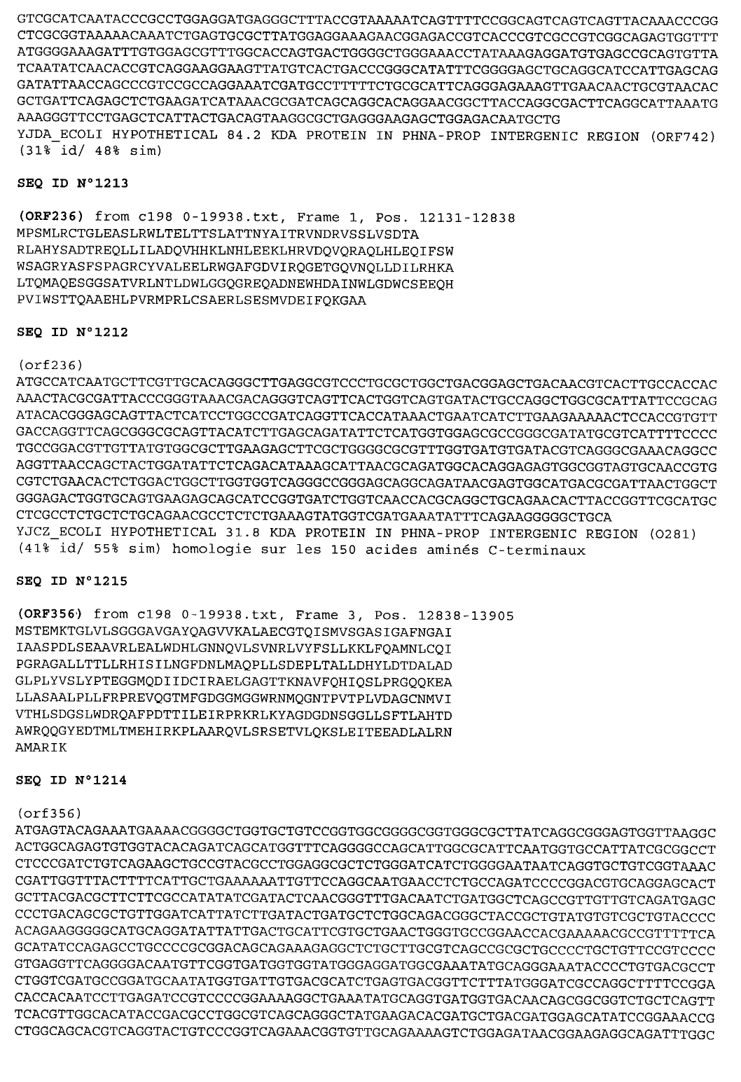

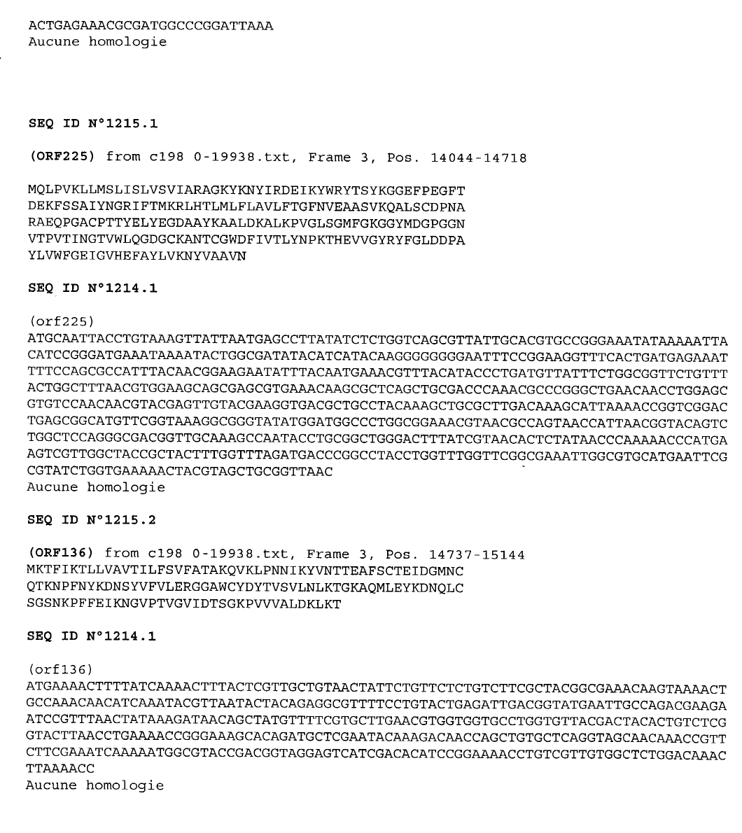

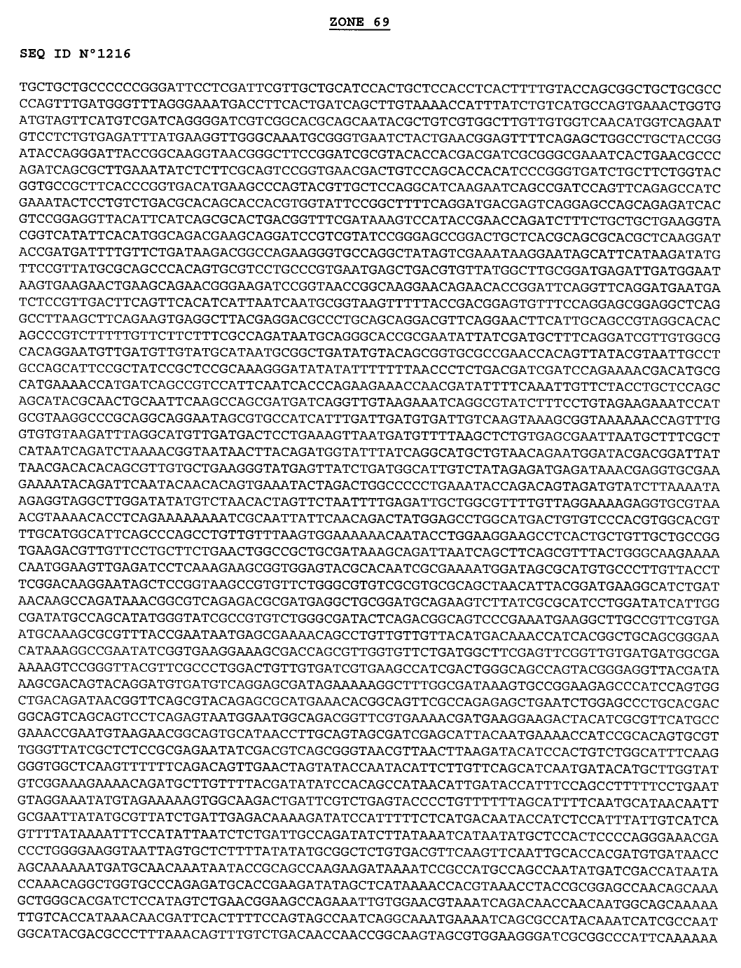

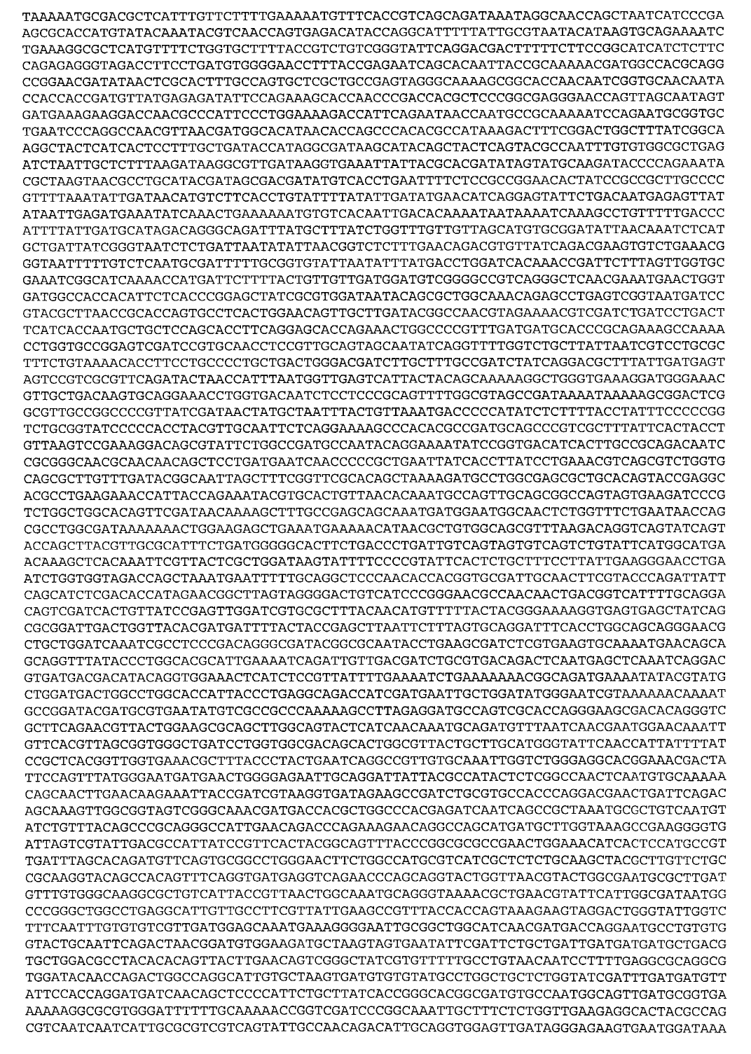

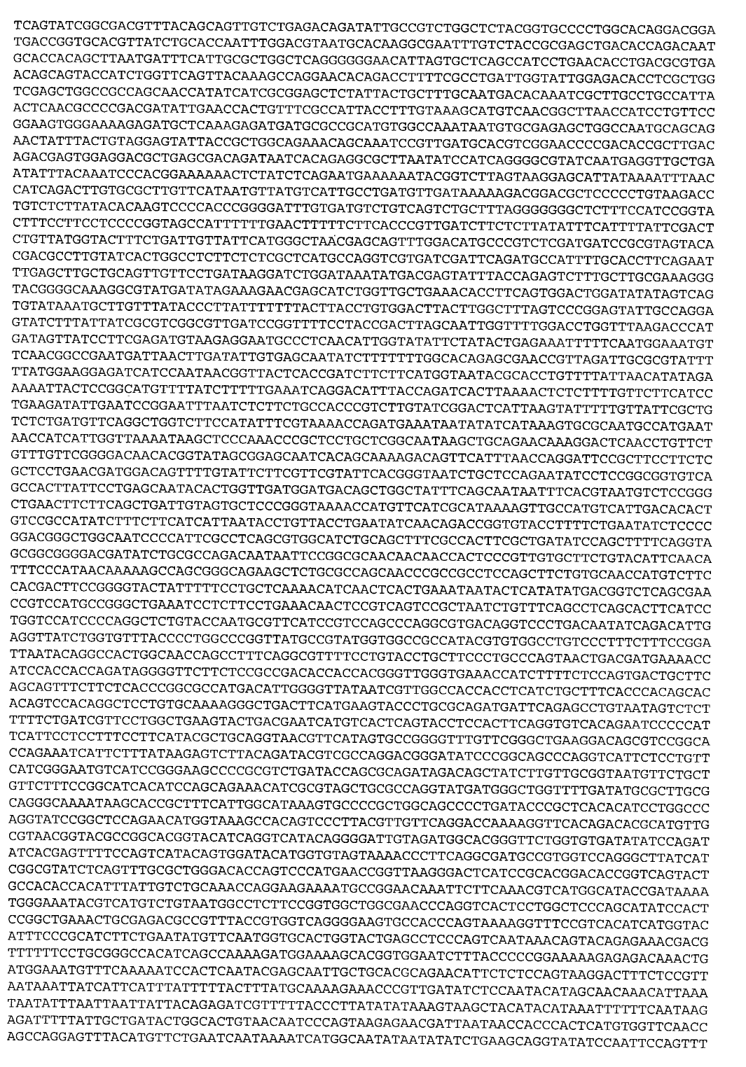

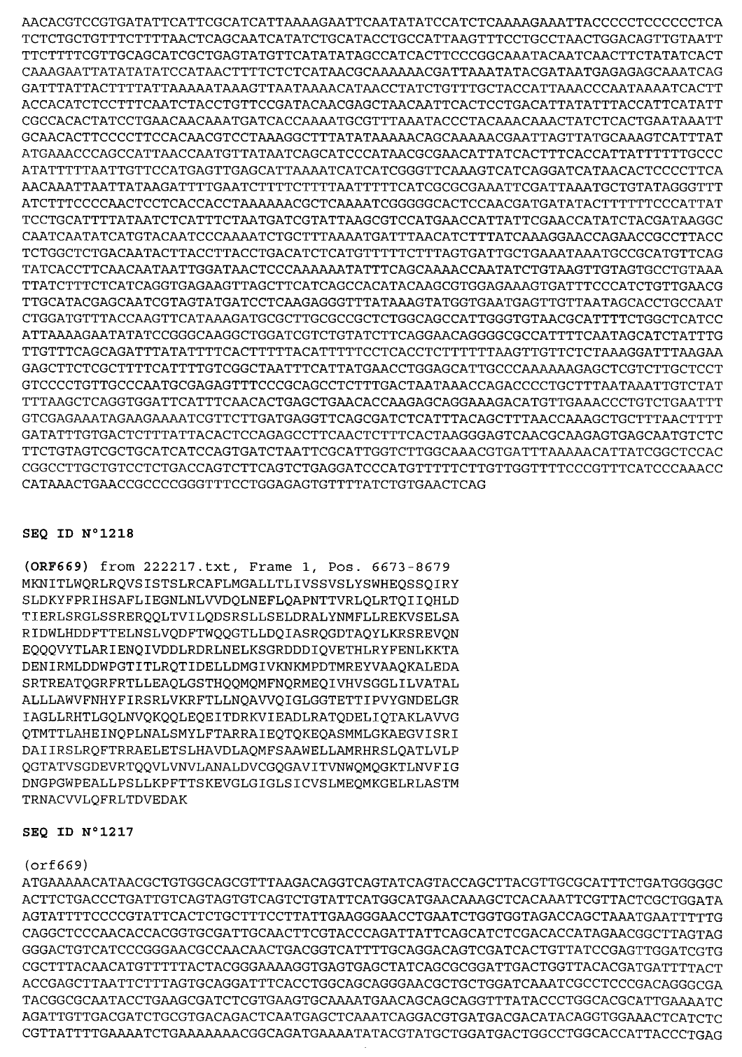

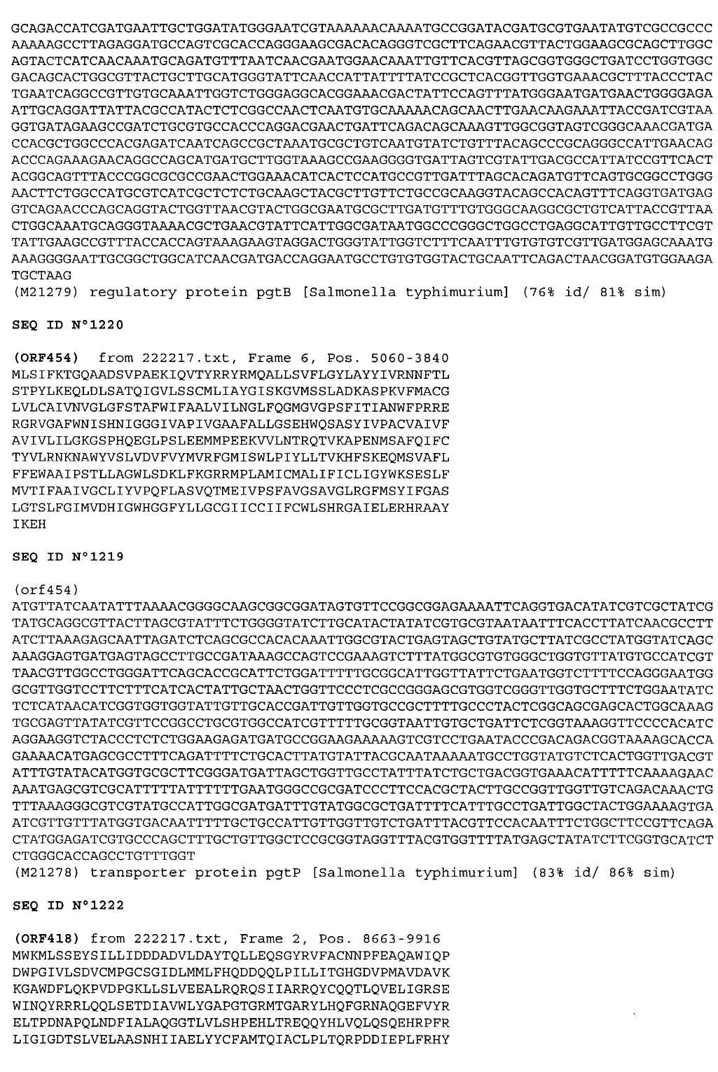

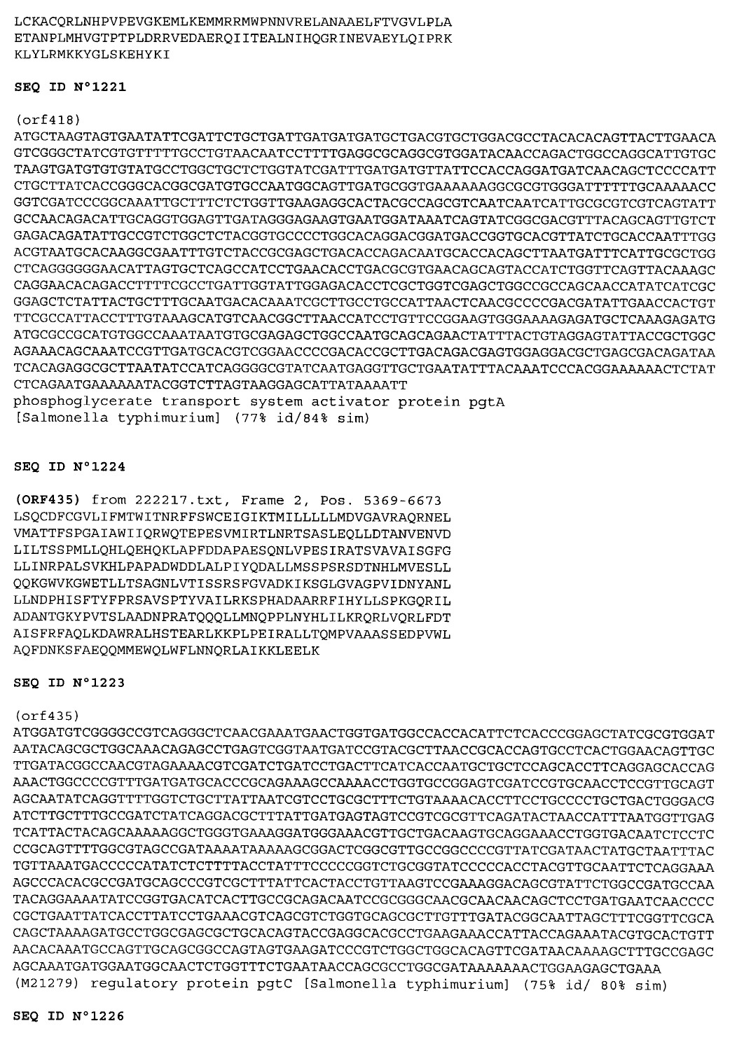

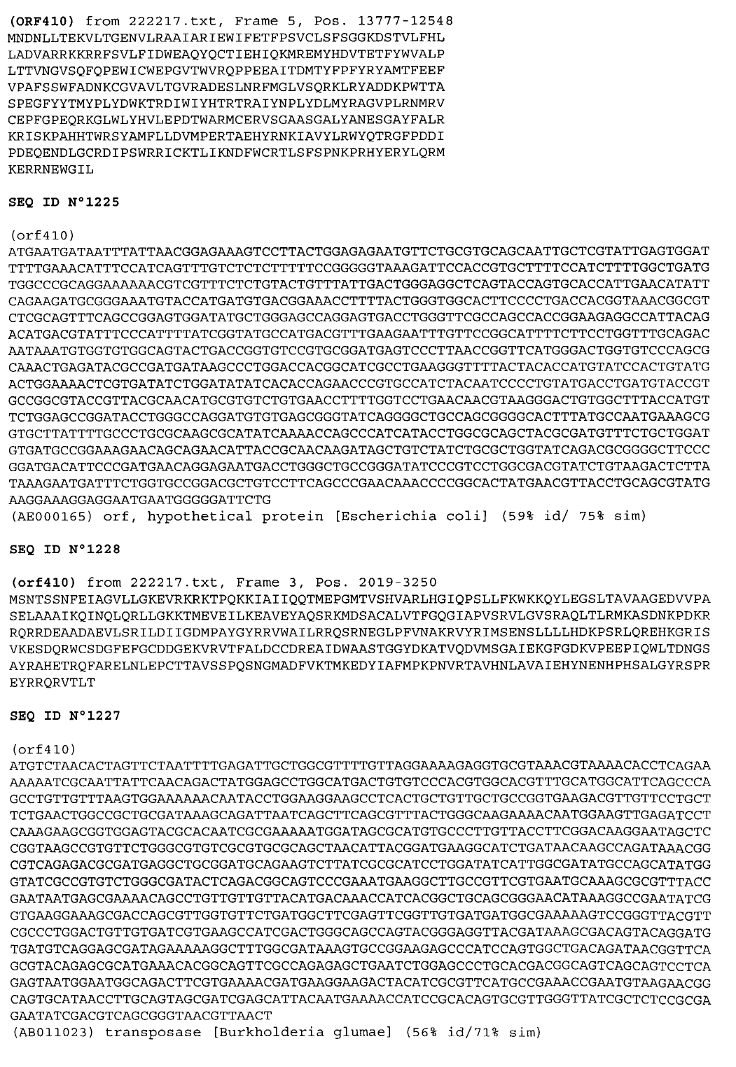

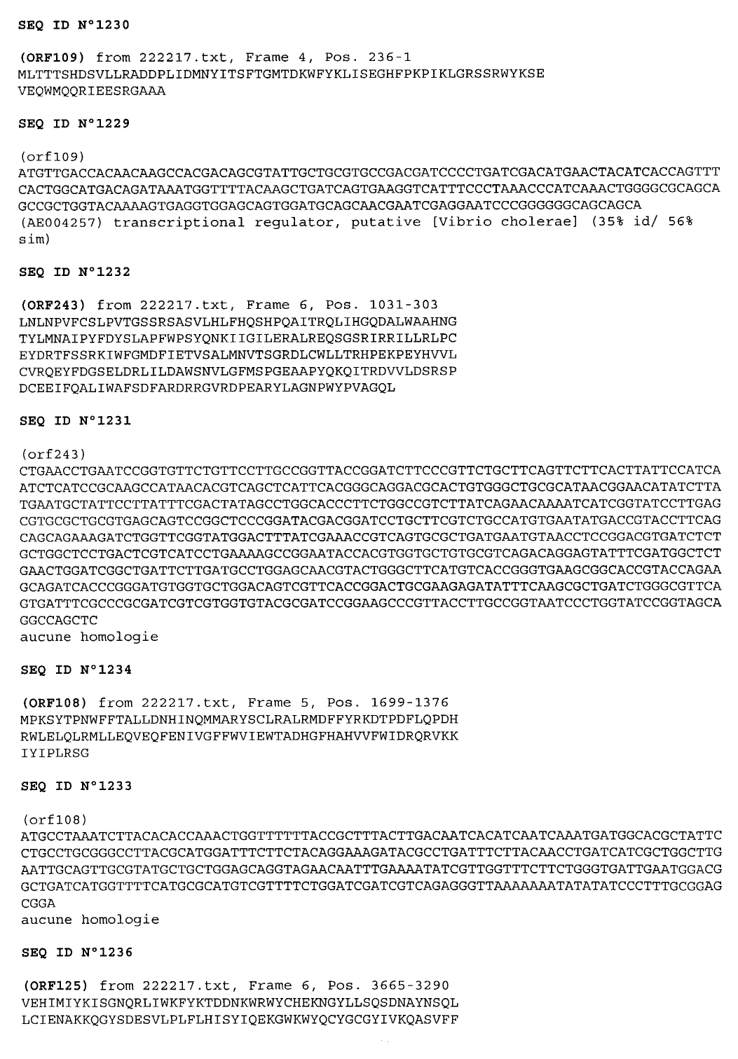

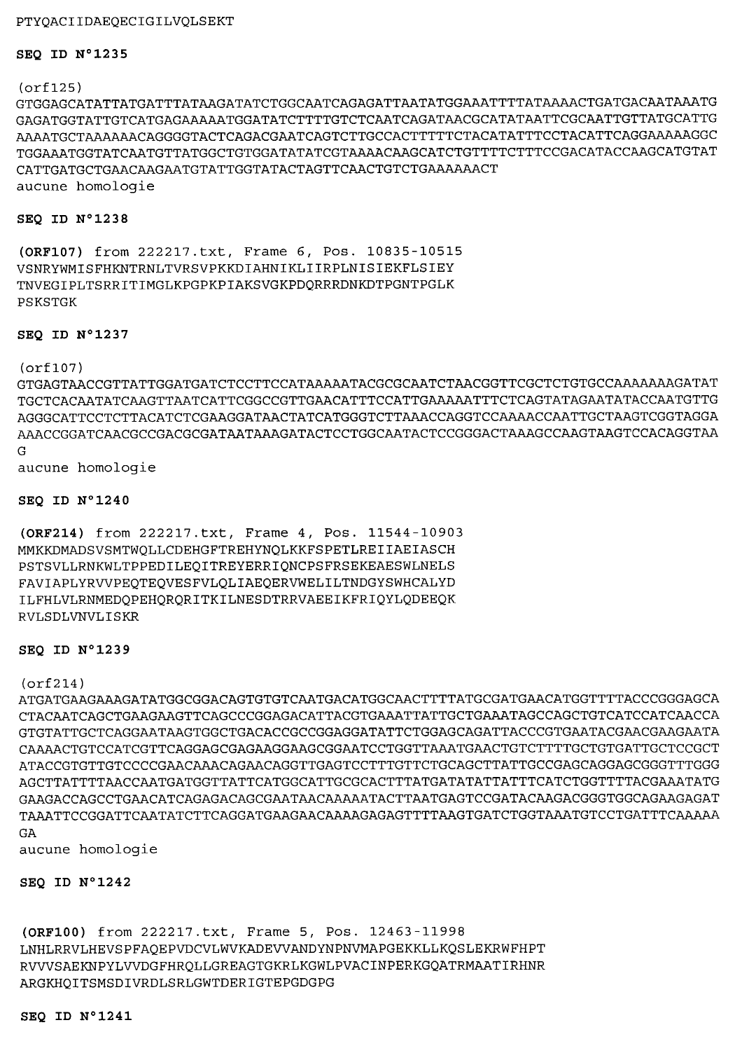

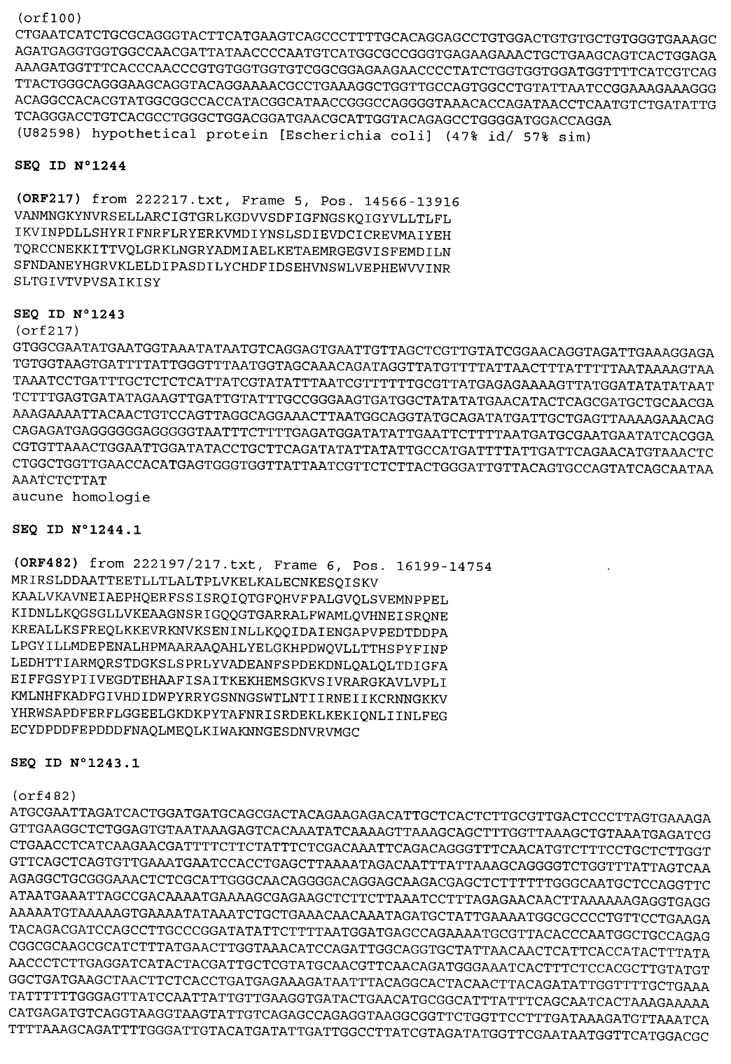

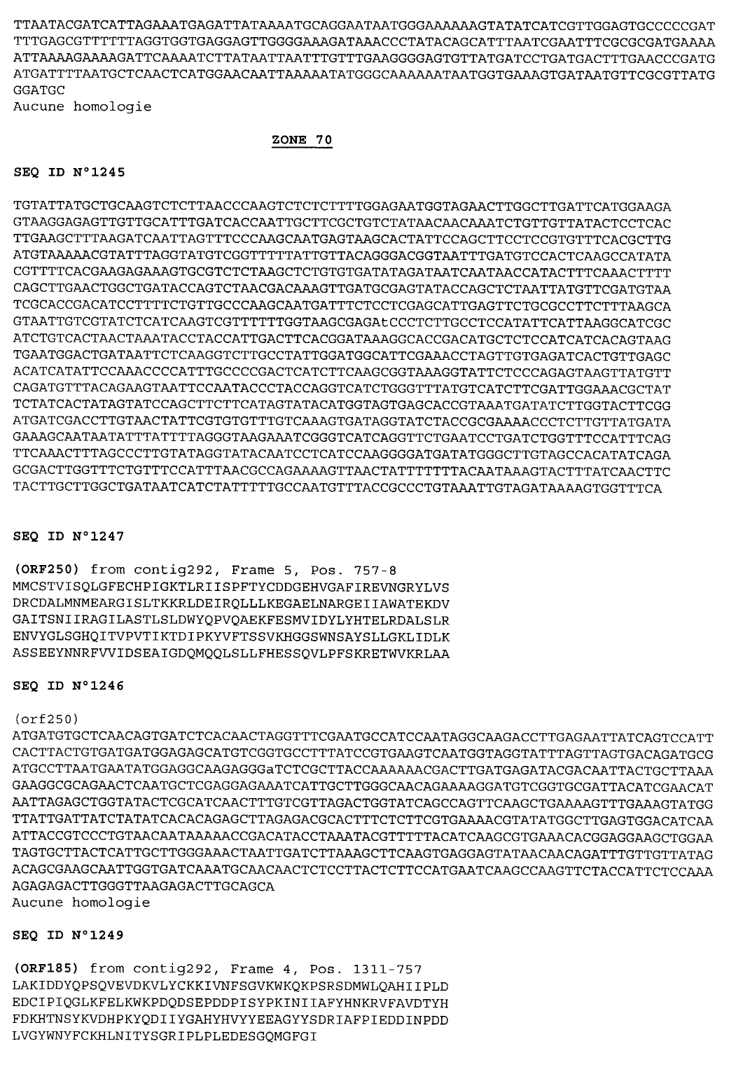

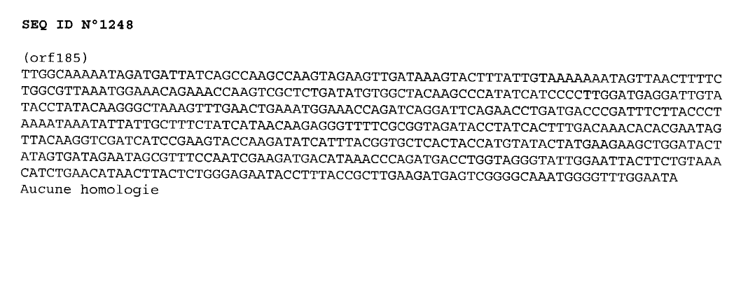

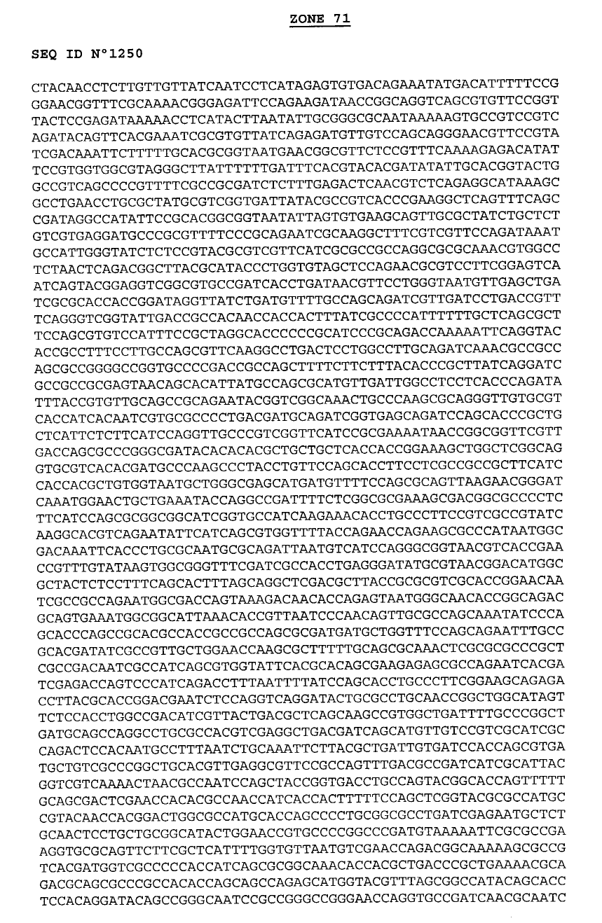

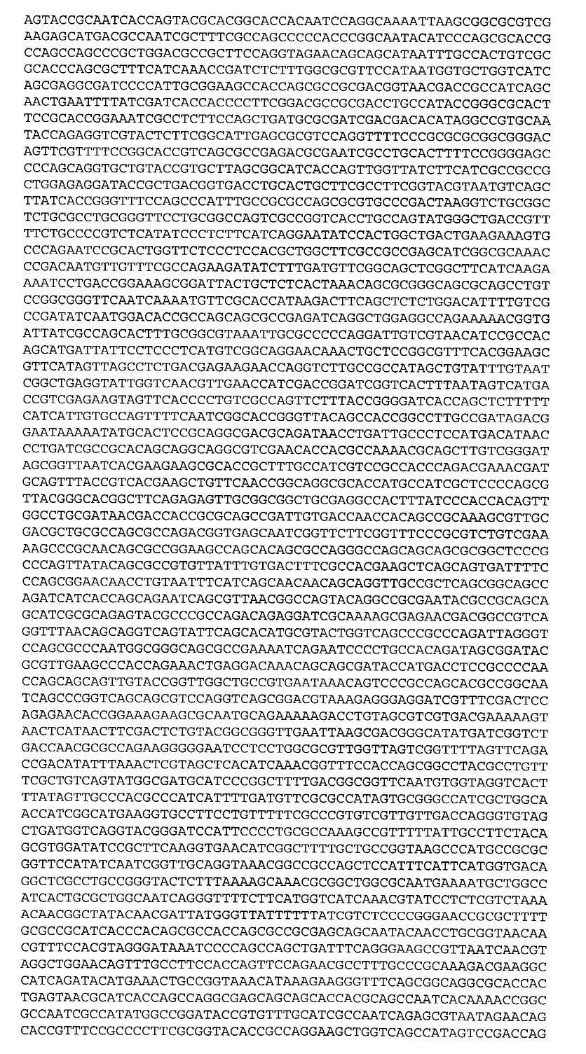

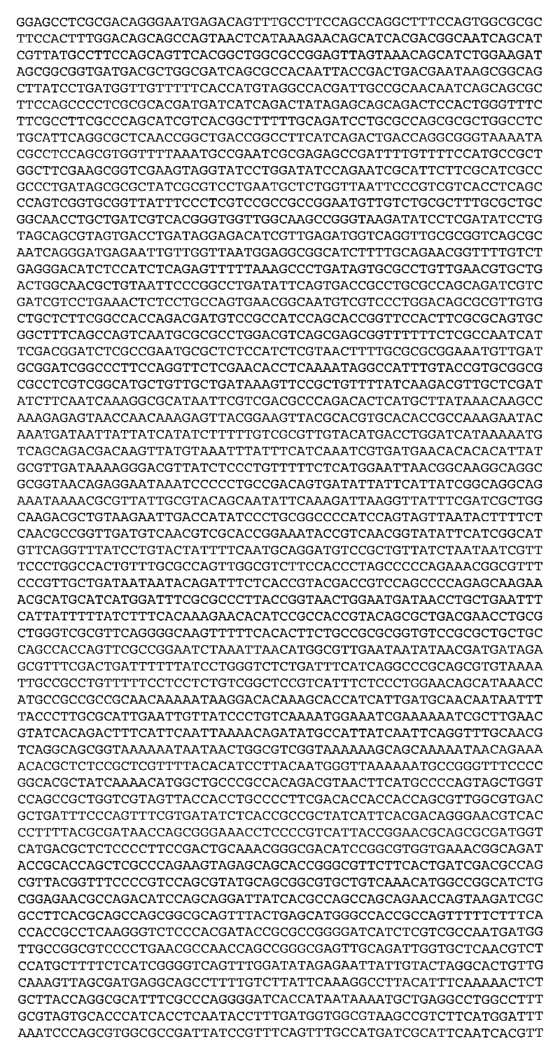

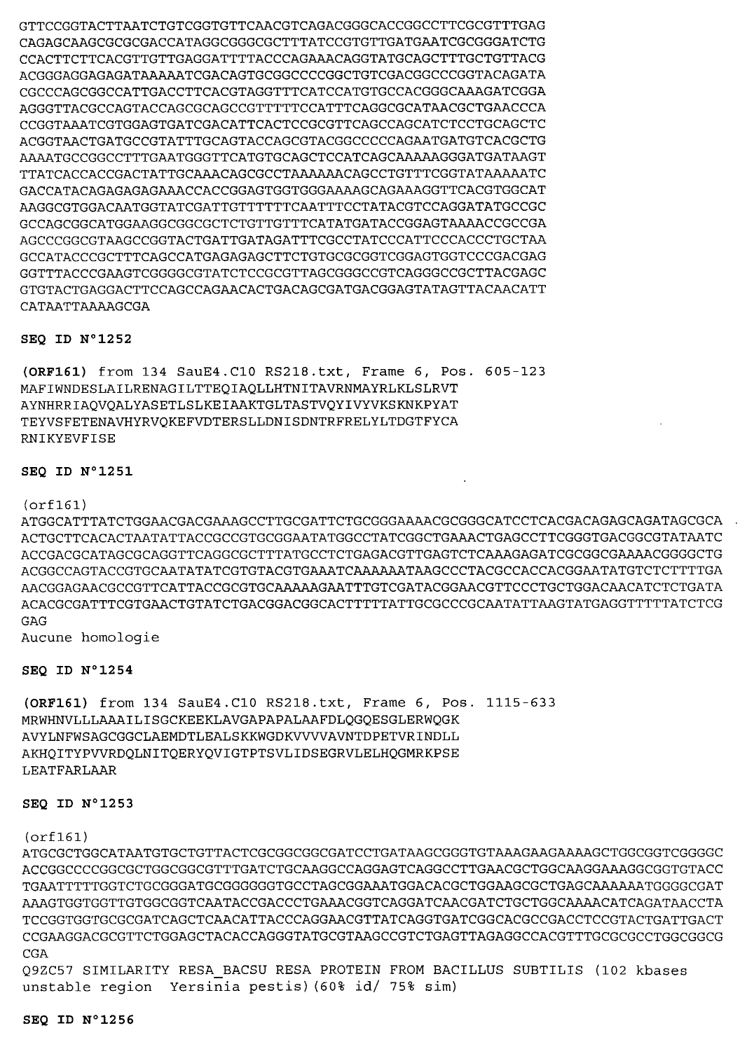

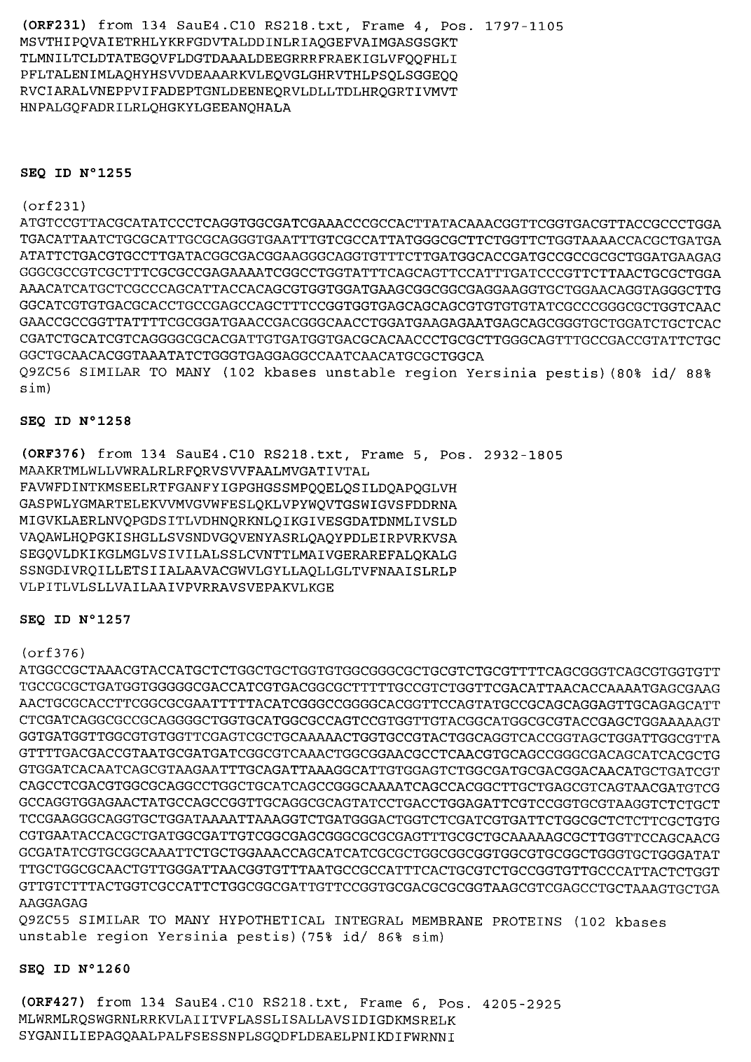

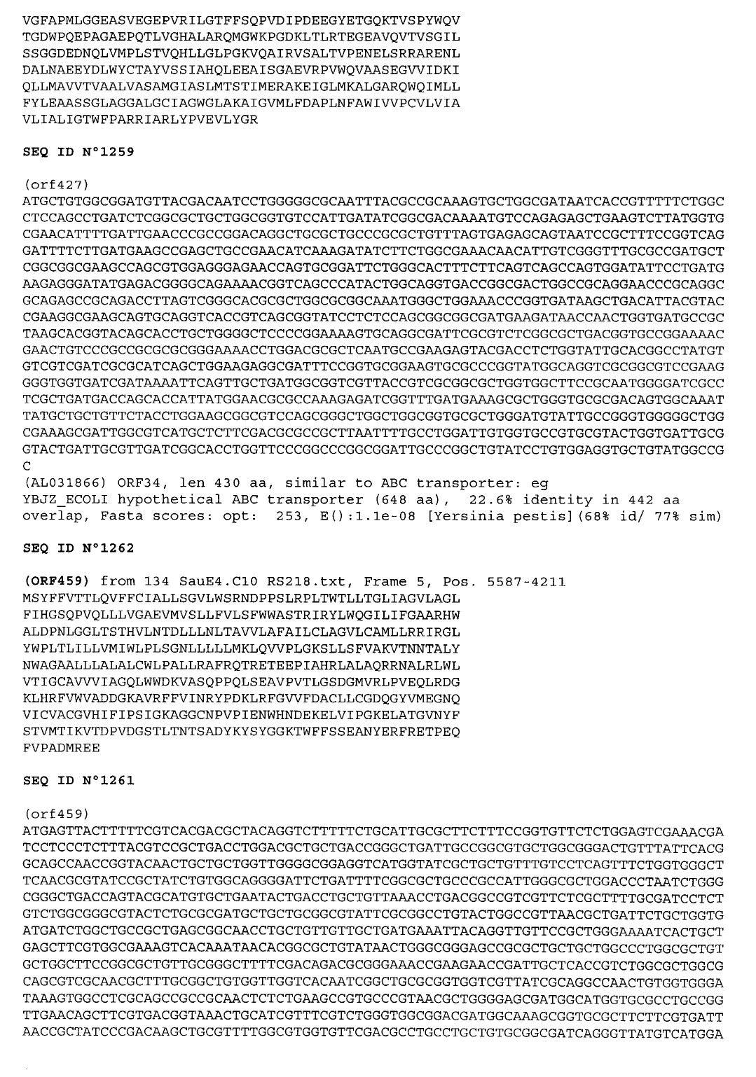

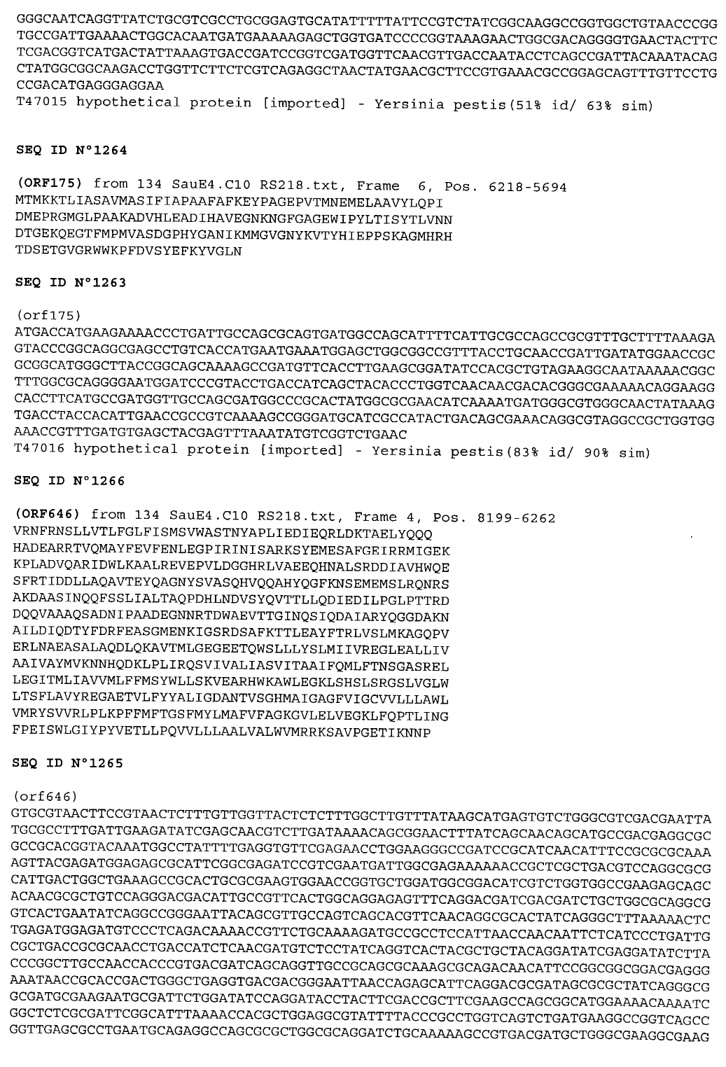

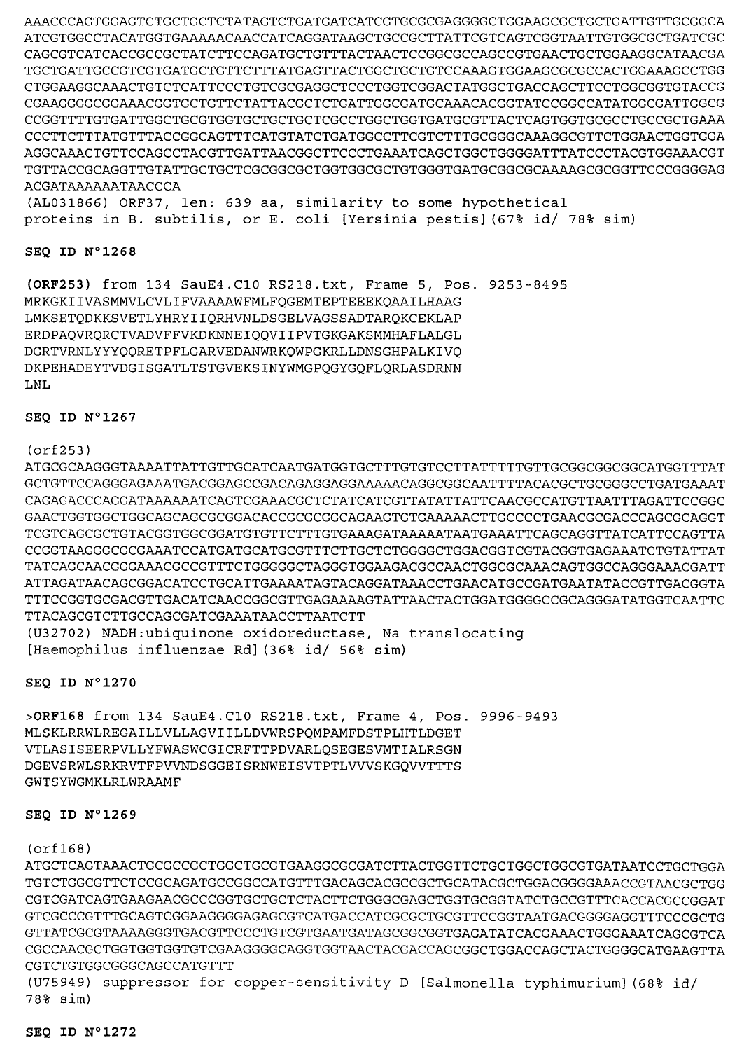

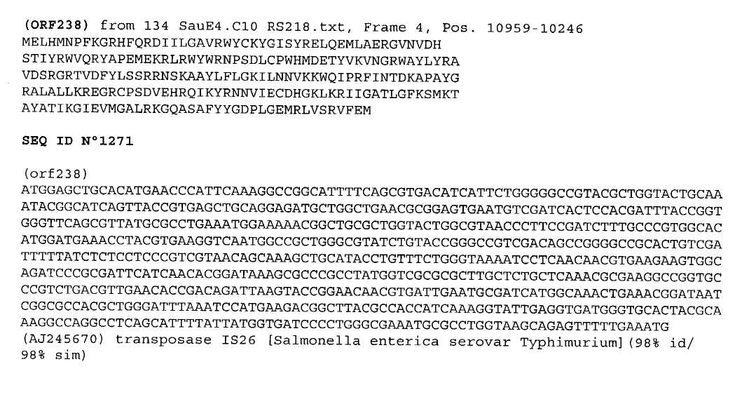

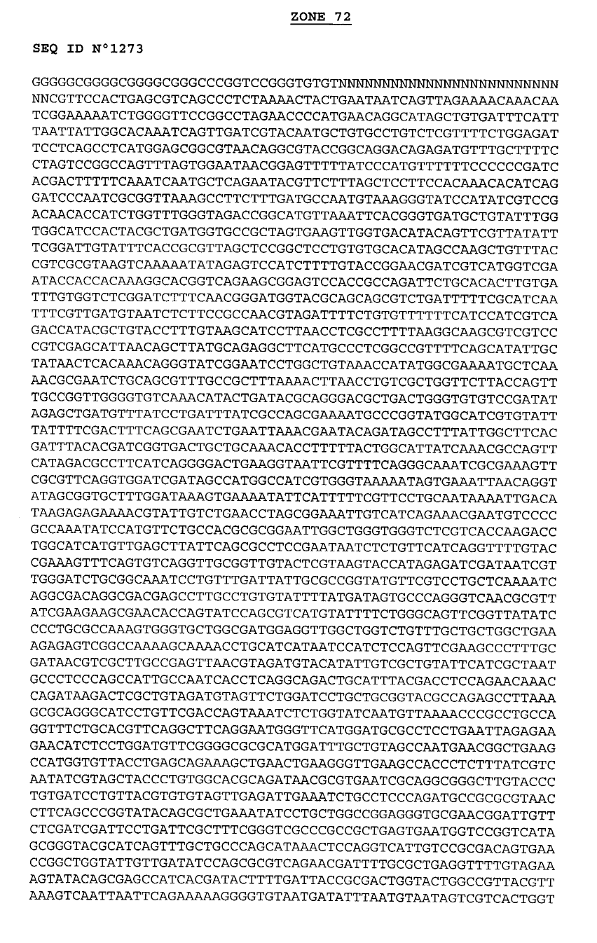

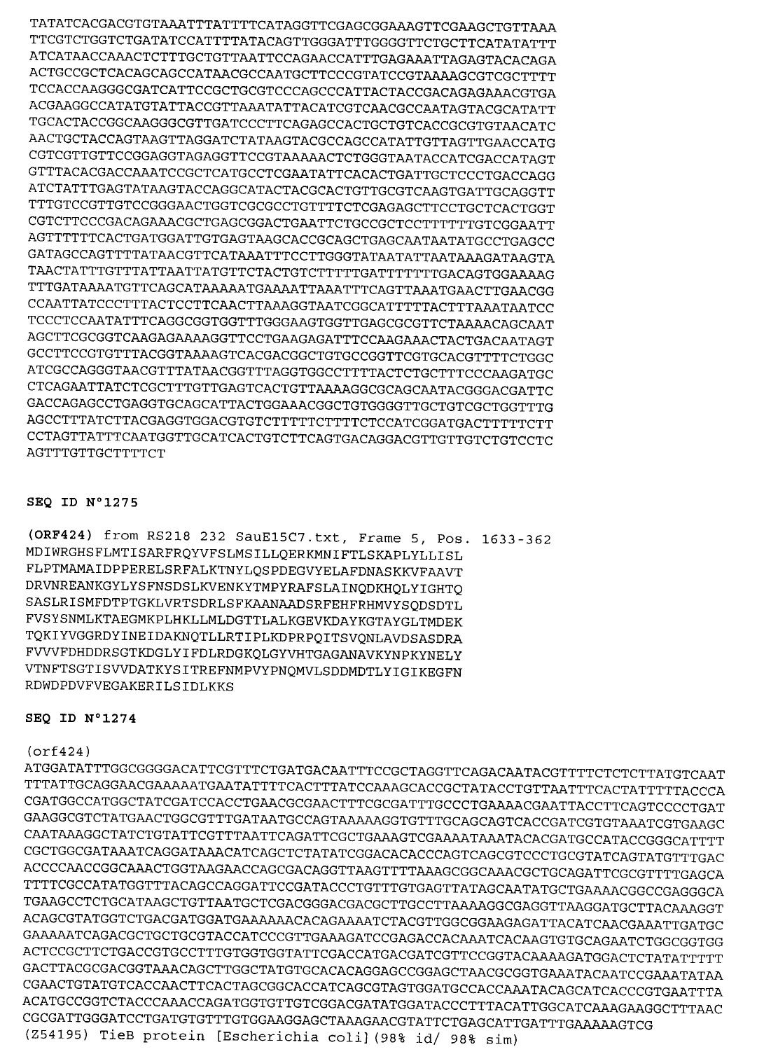

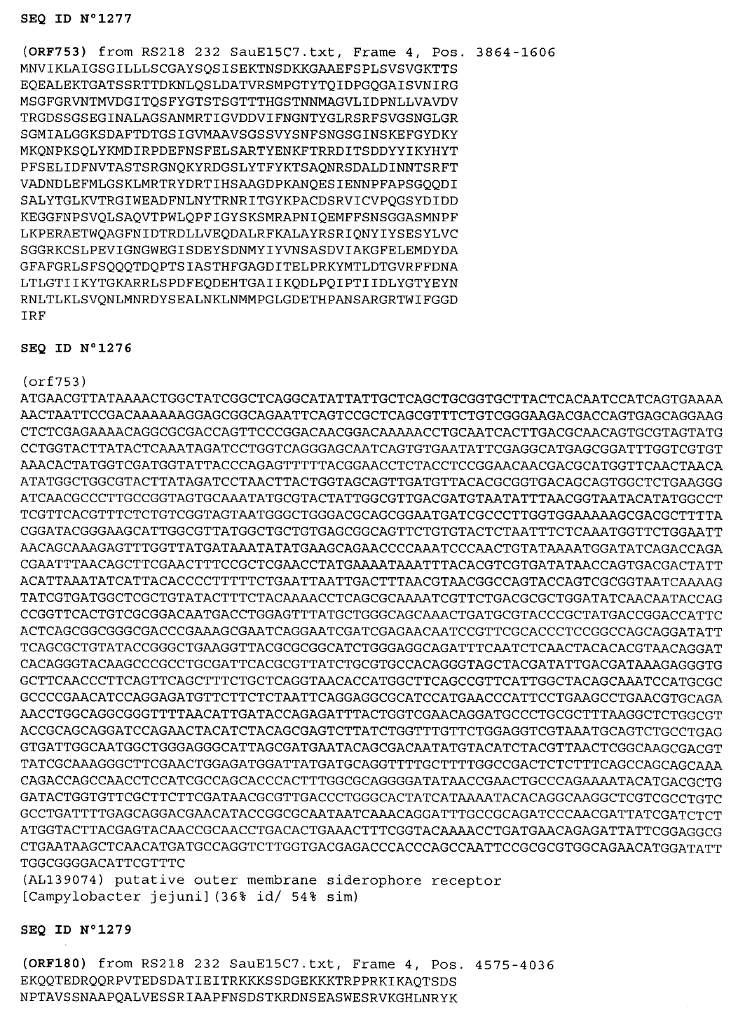

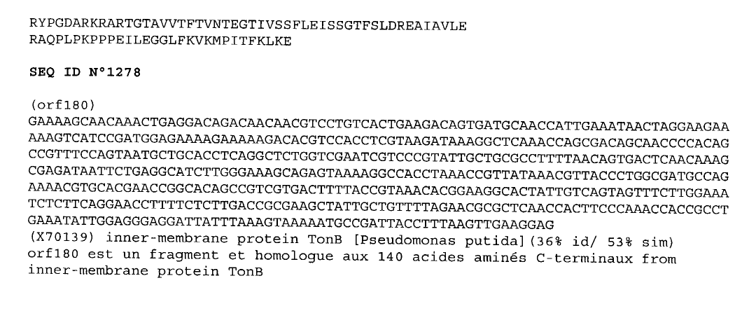

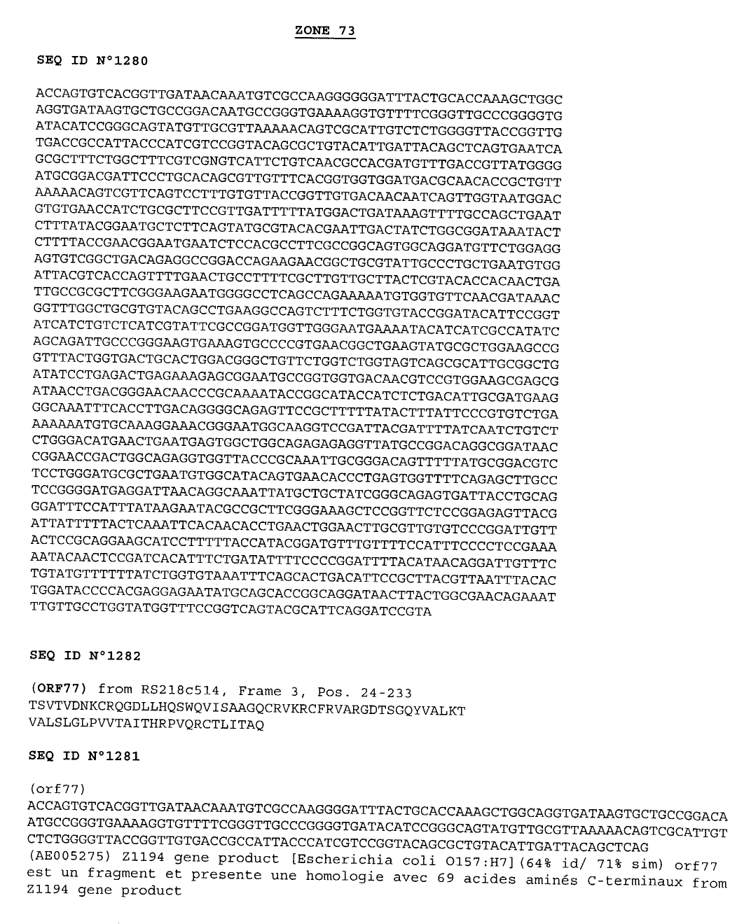

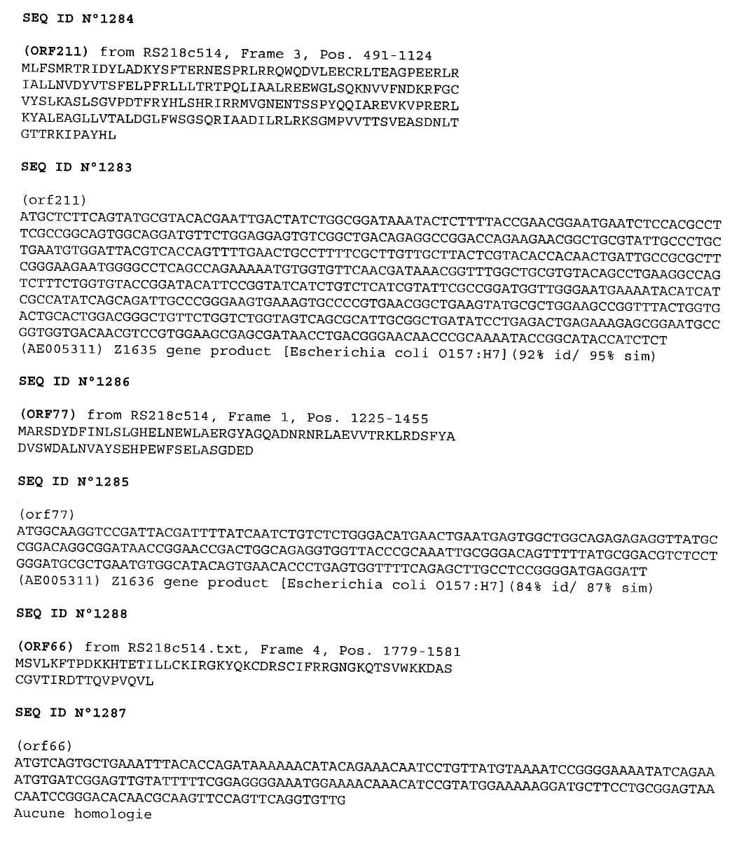

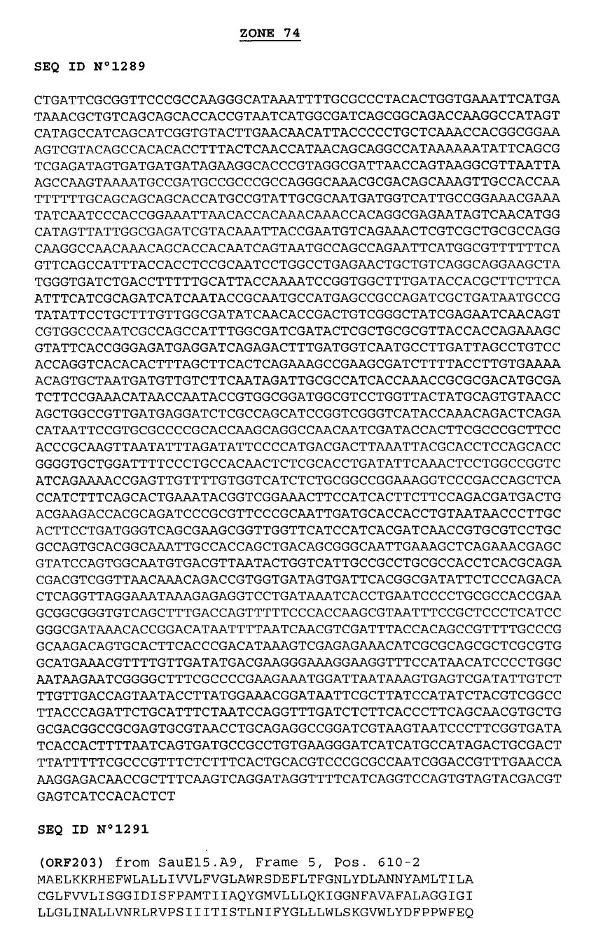

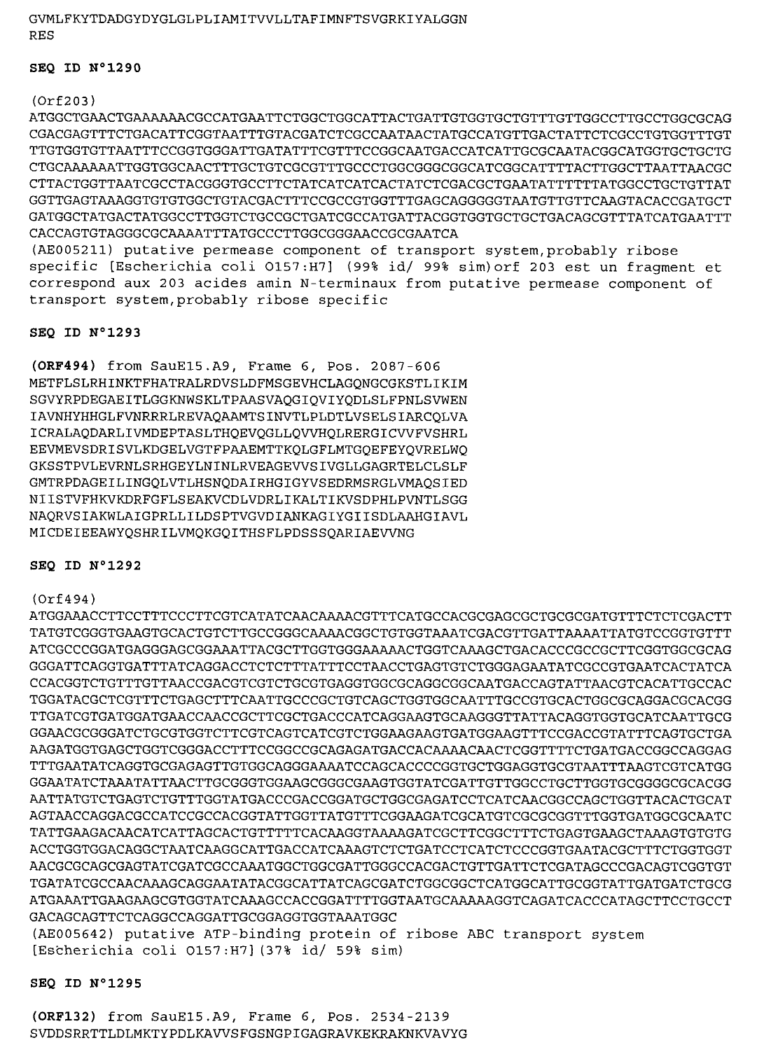

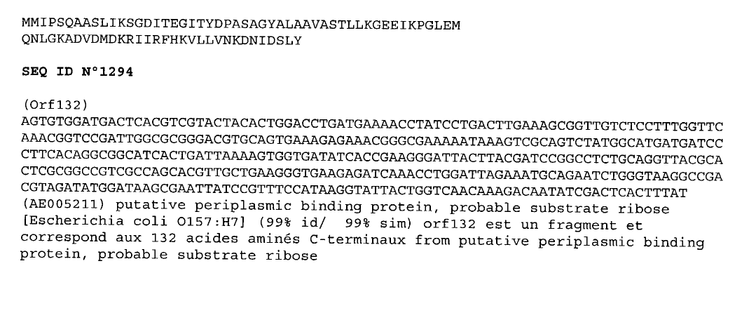

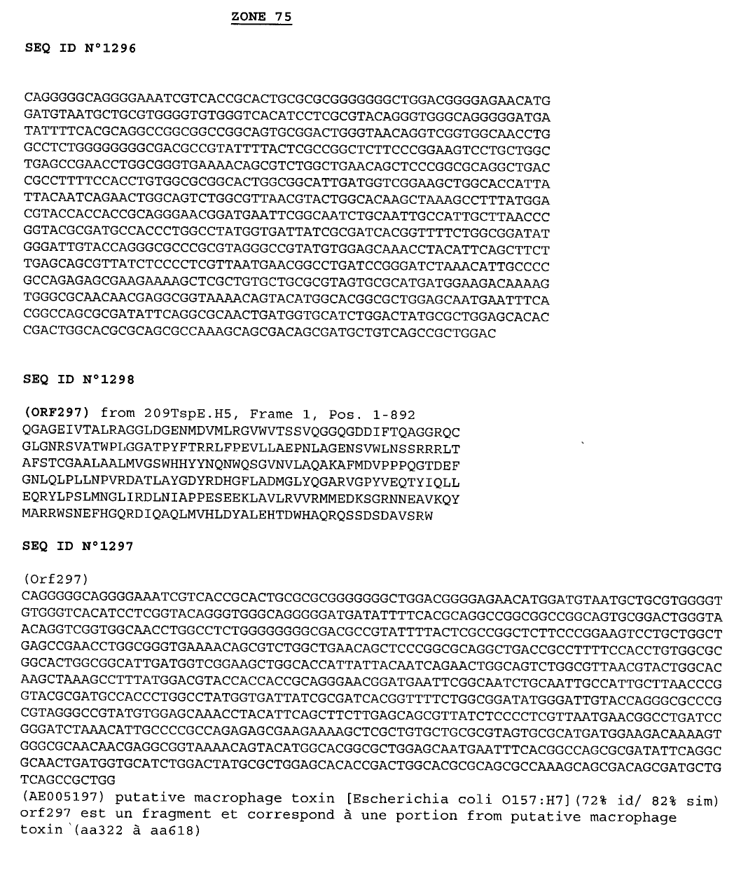

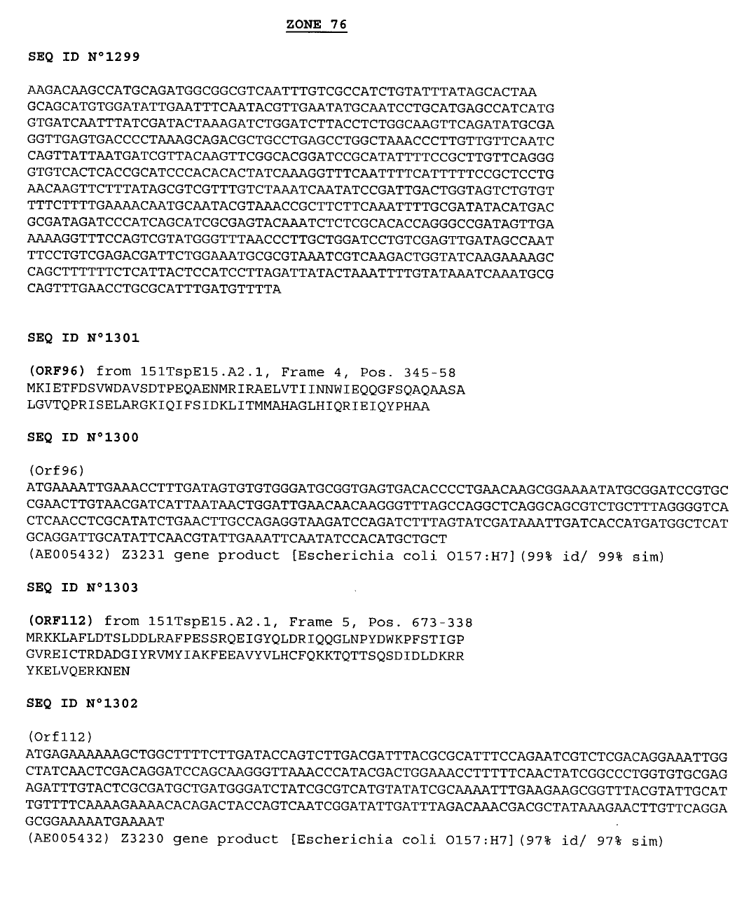

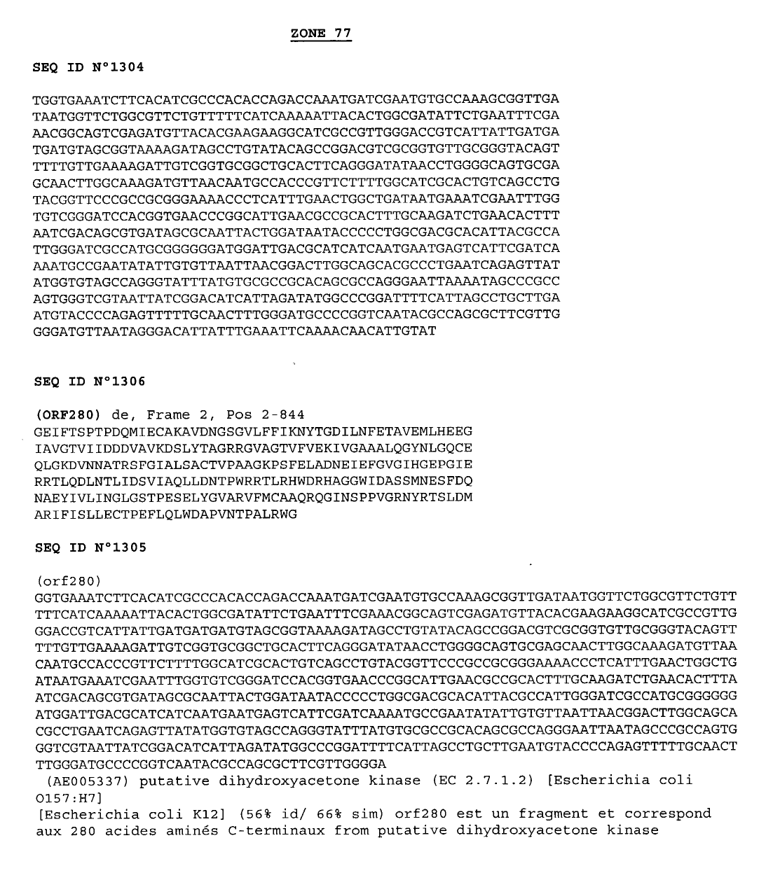

FIG. 6 represents the sequences of the CTF073+ K12− regions and RS218+ K12− regions obtained using the clones of FIG. 2,

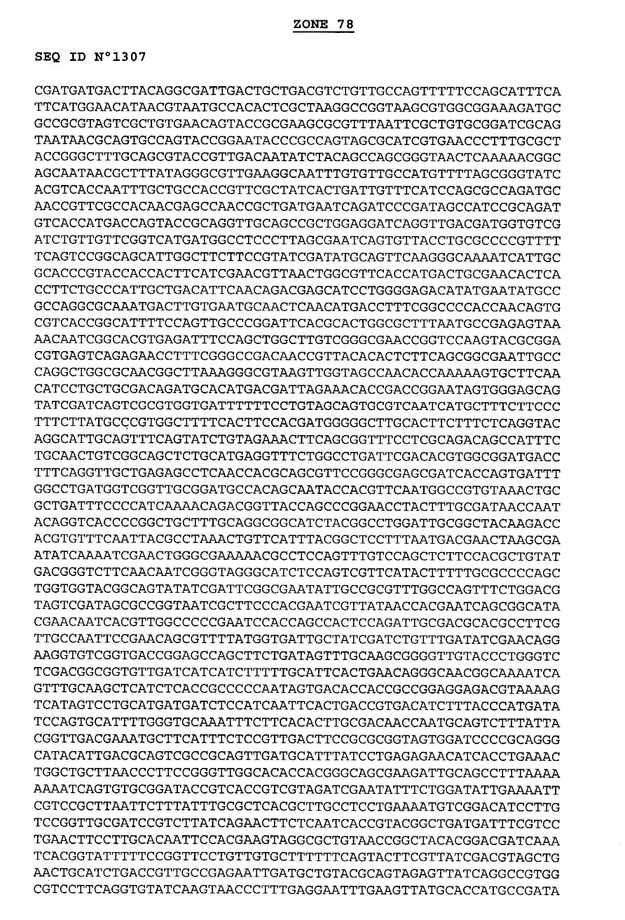

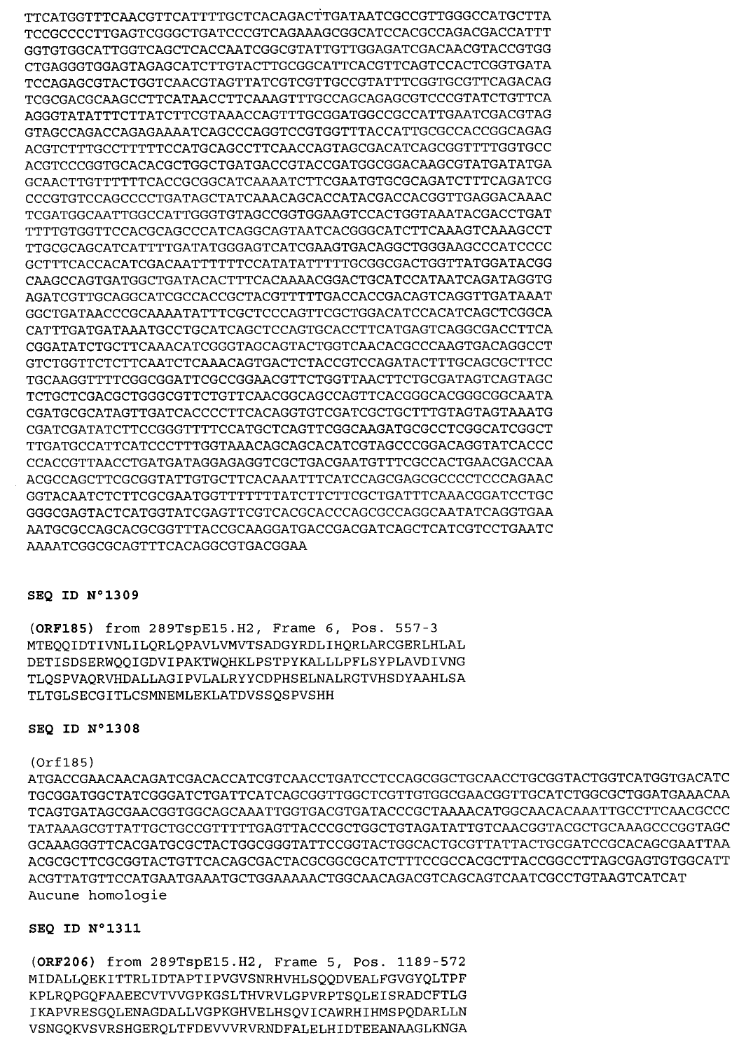

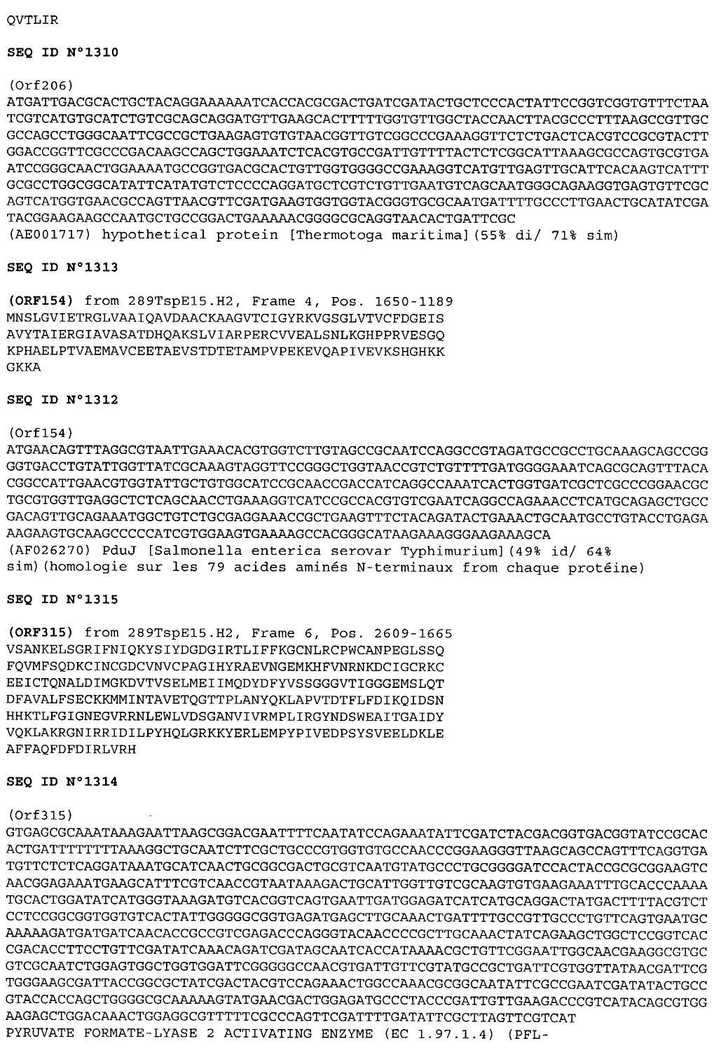

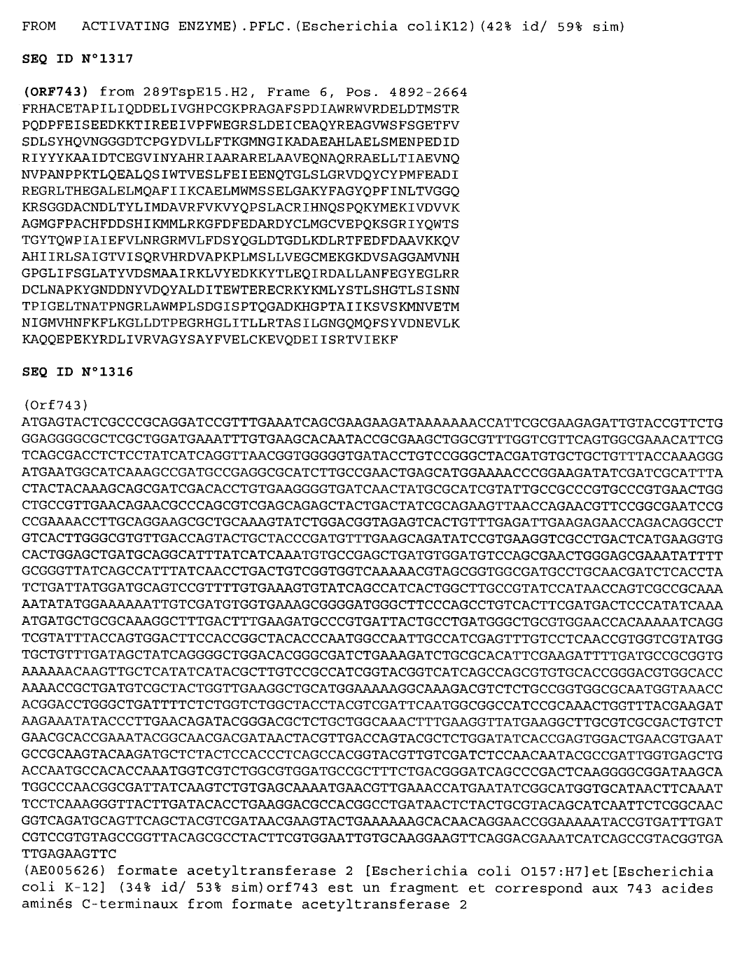

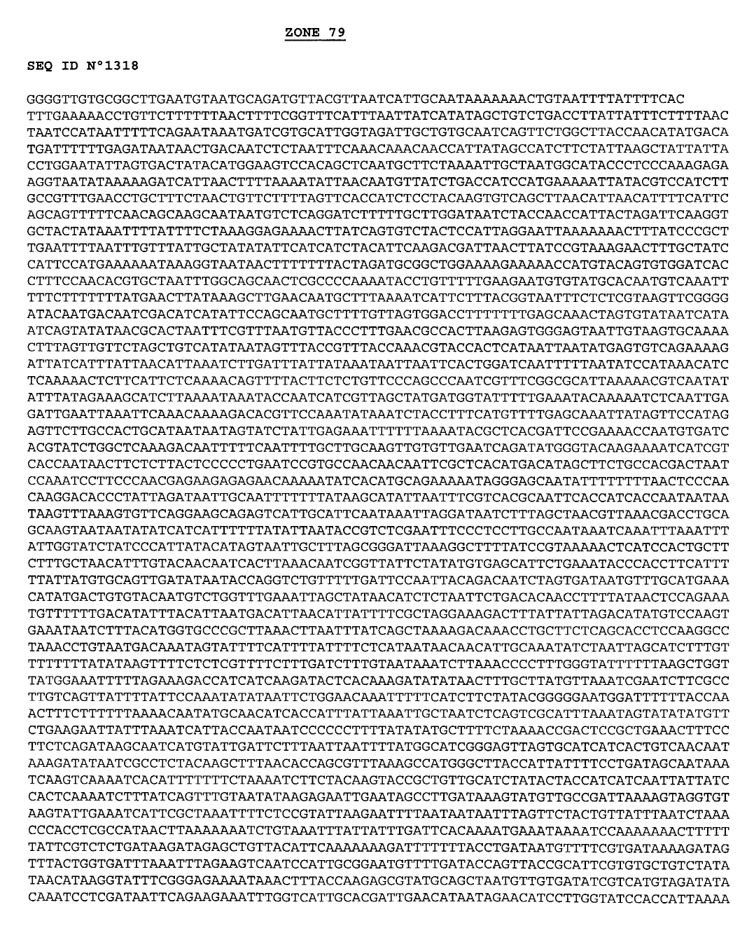

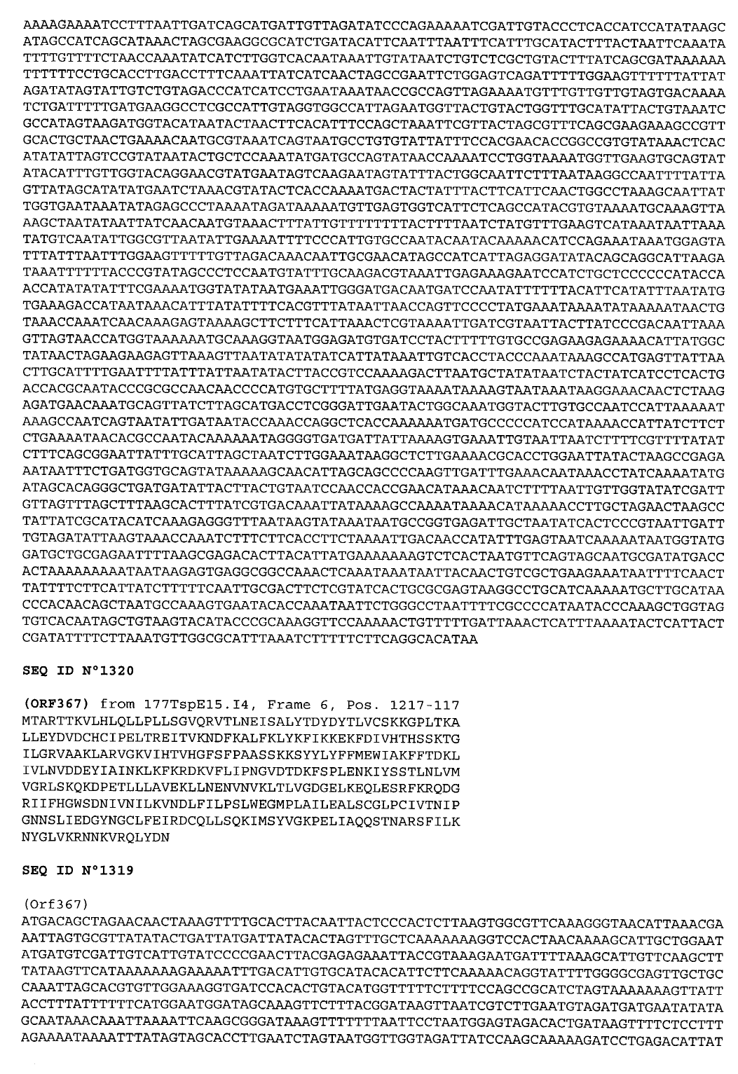

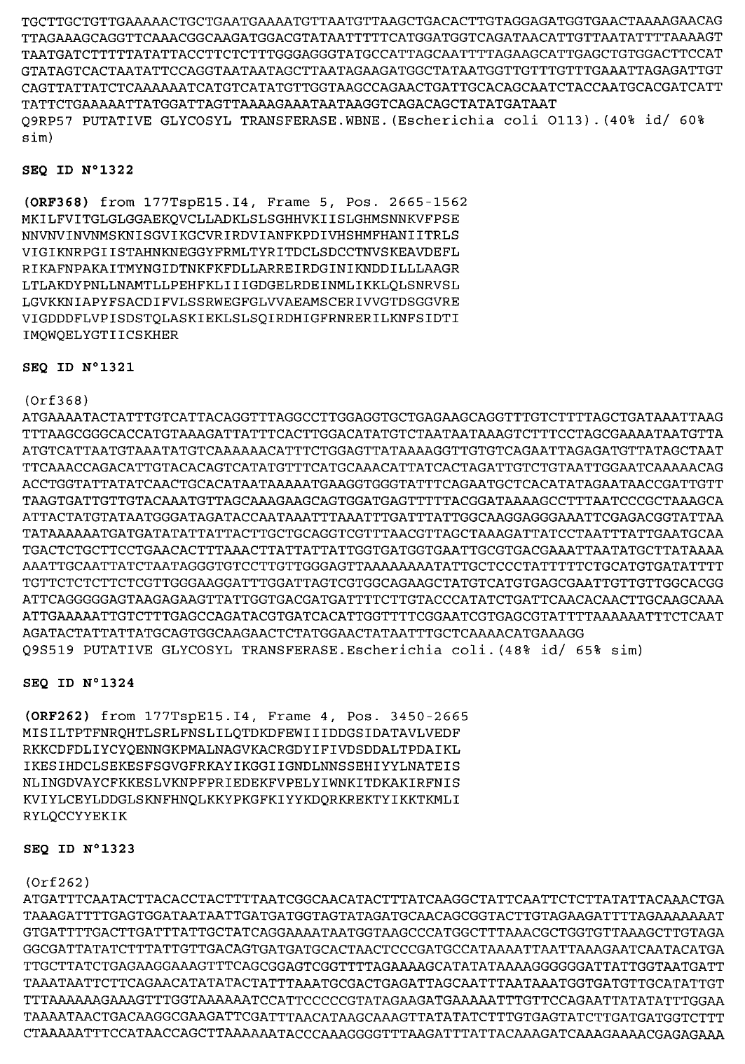

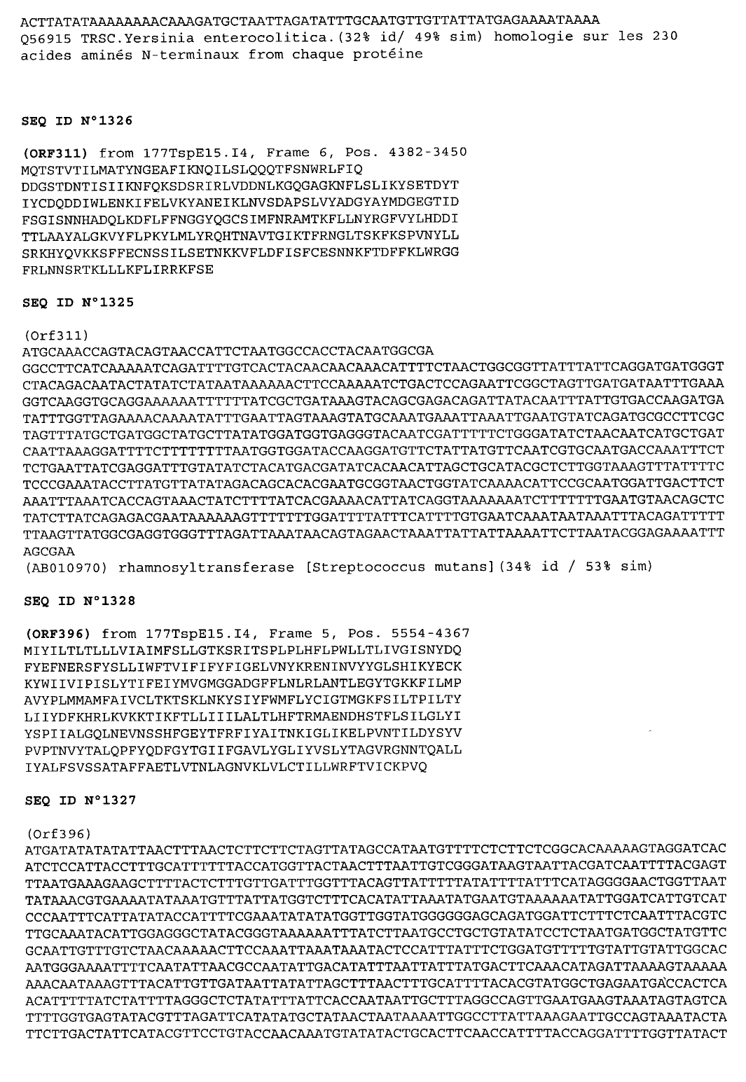

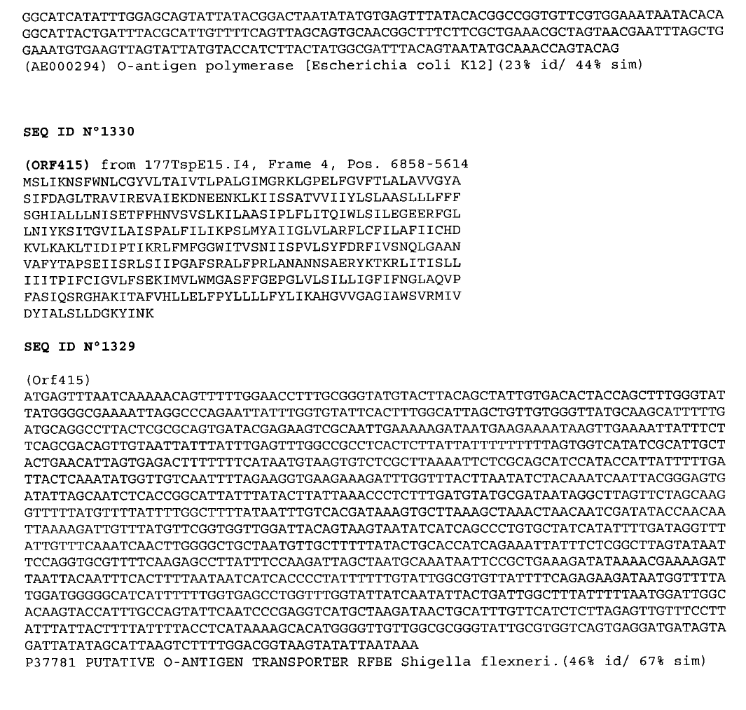

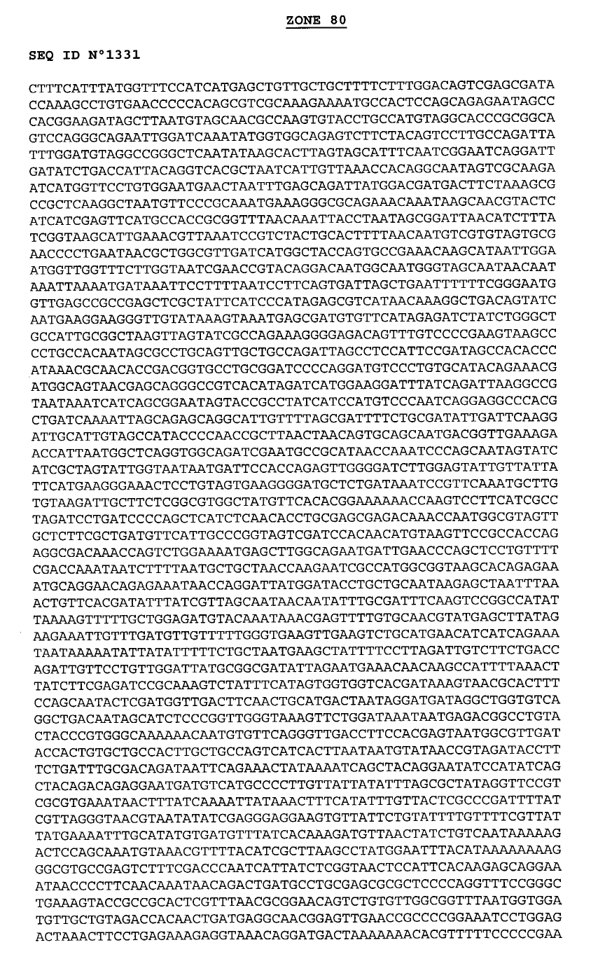

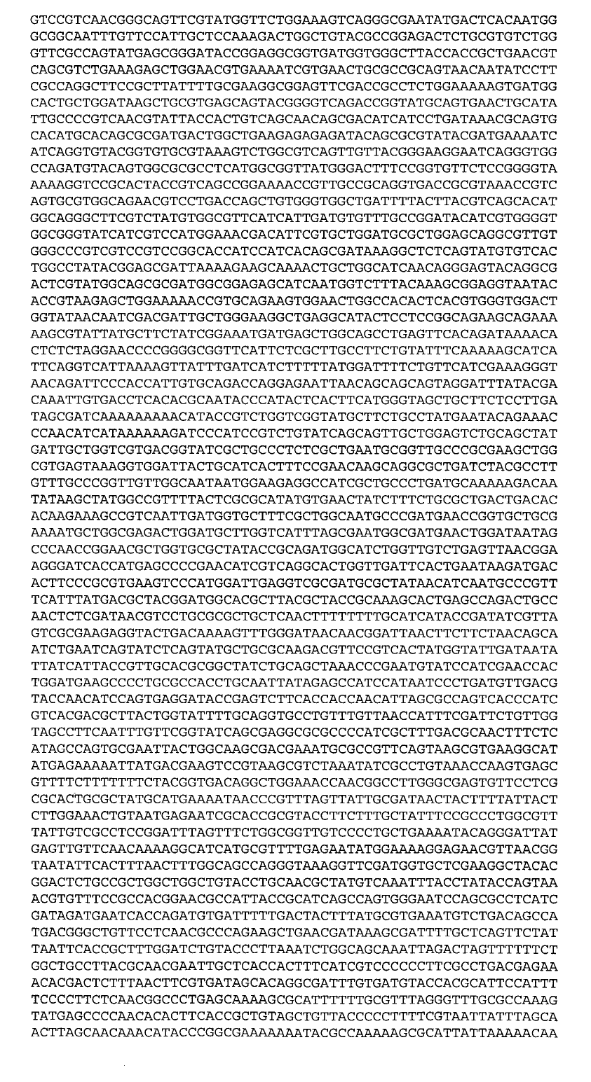

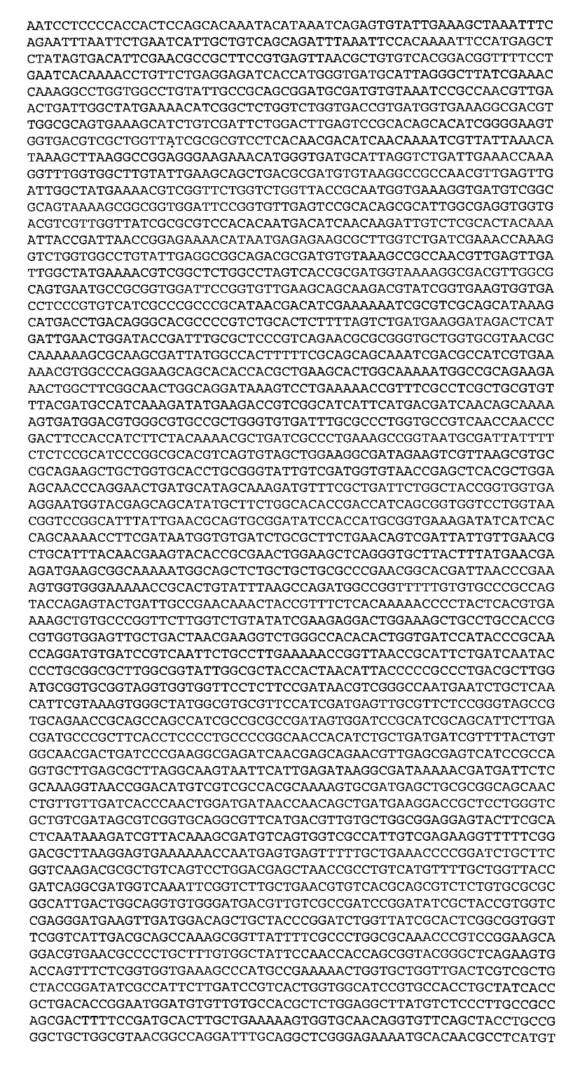

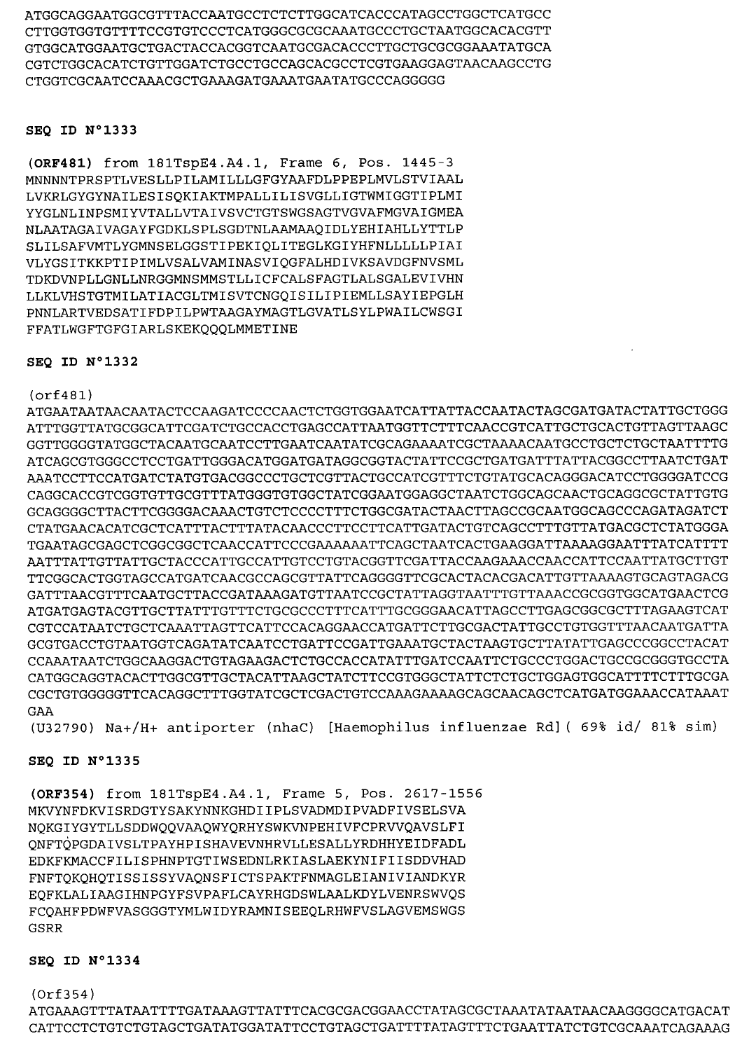

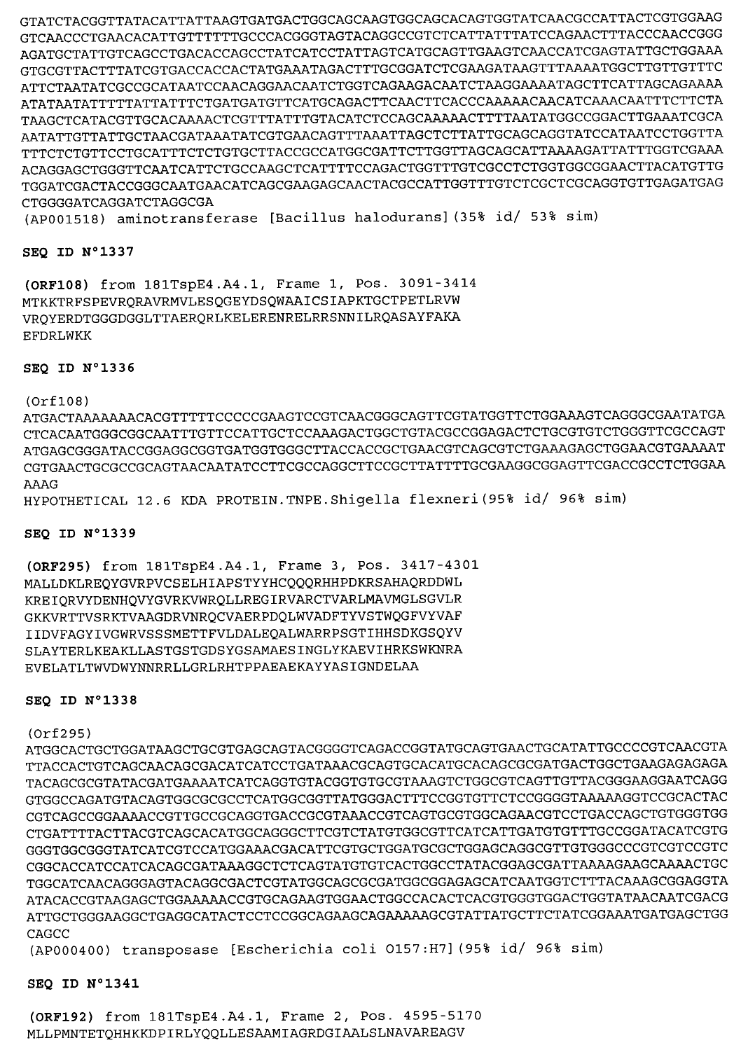

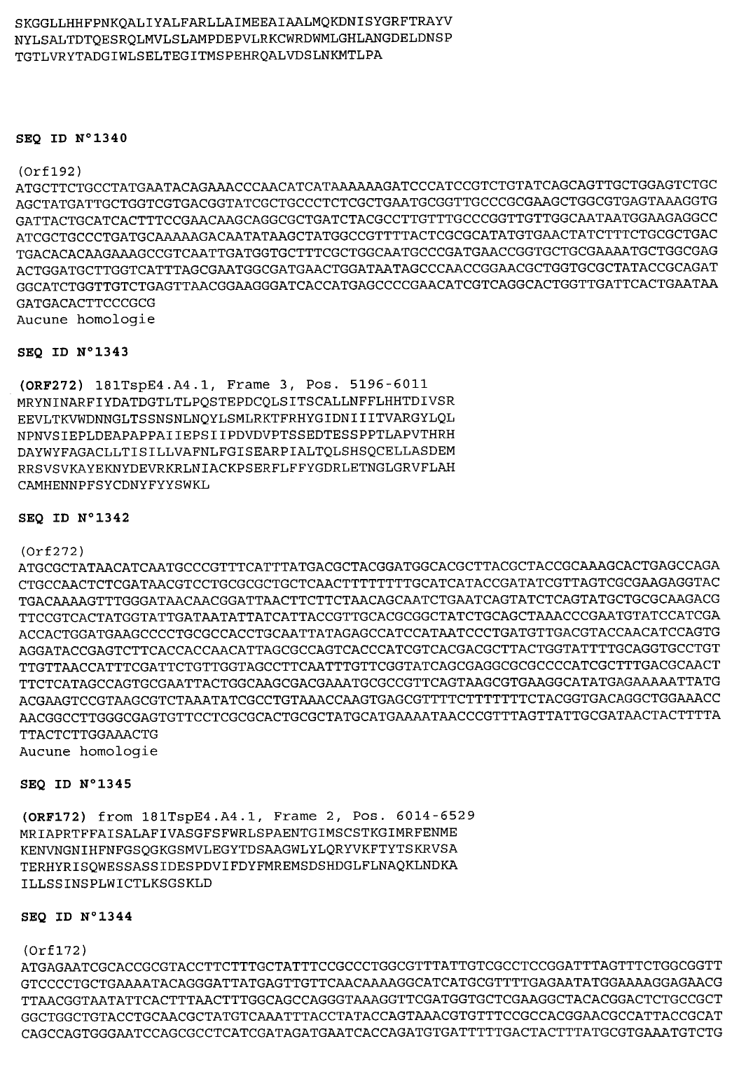

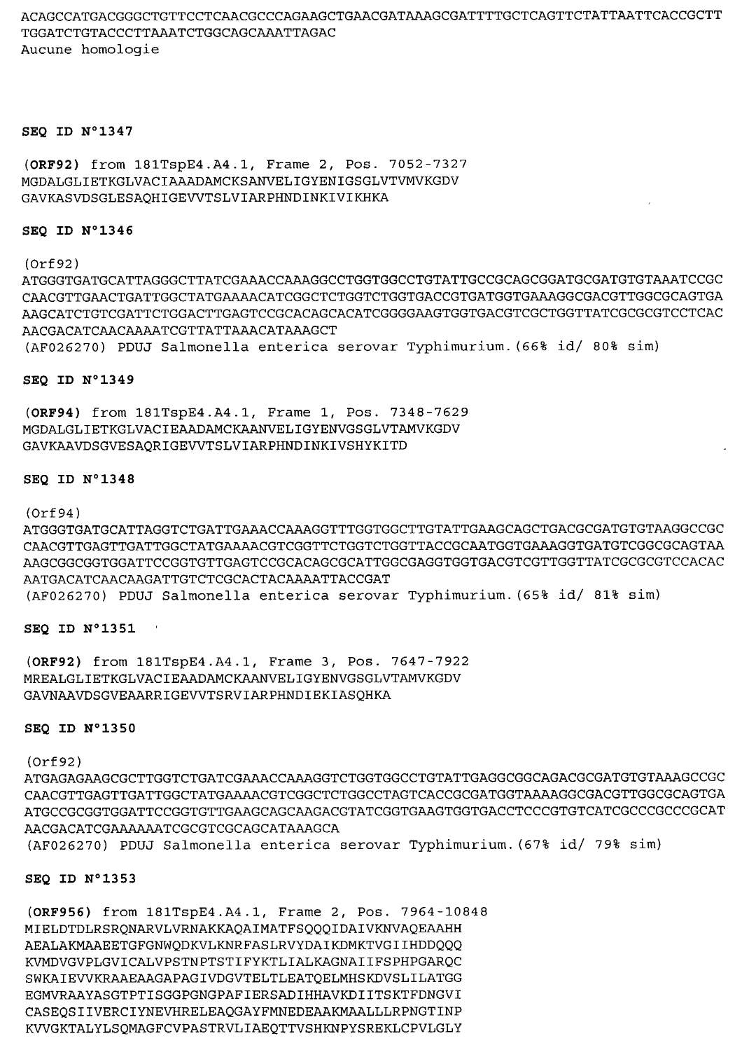

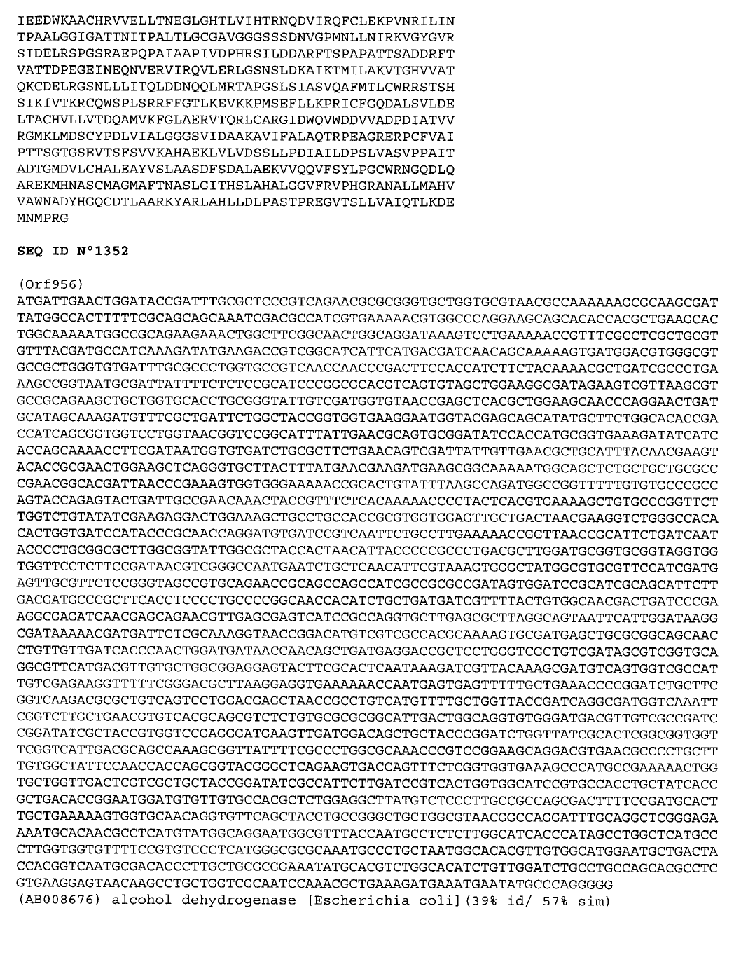

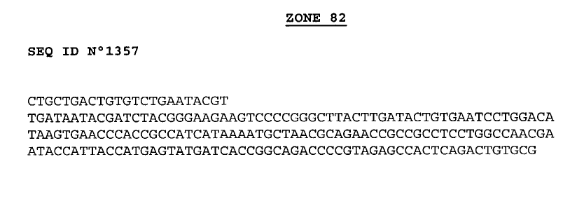

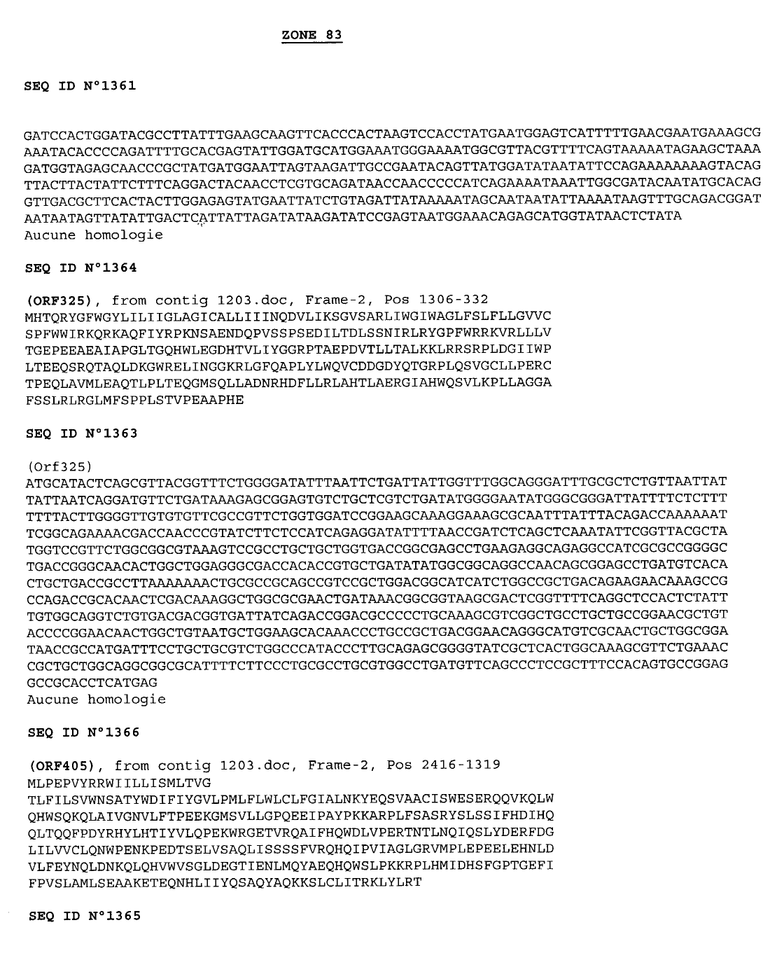

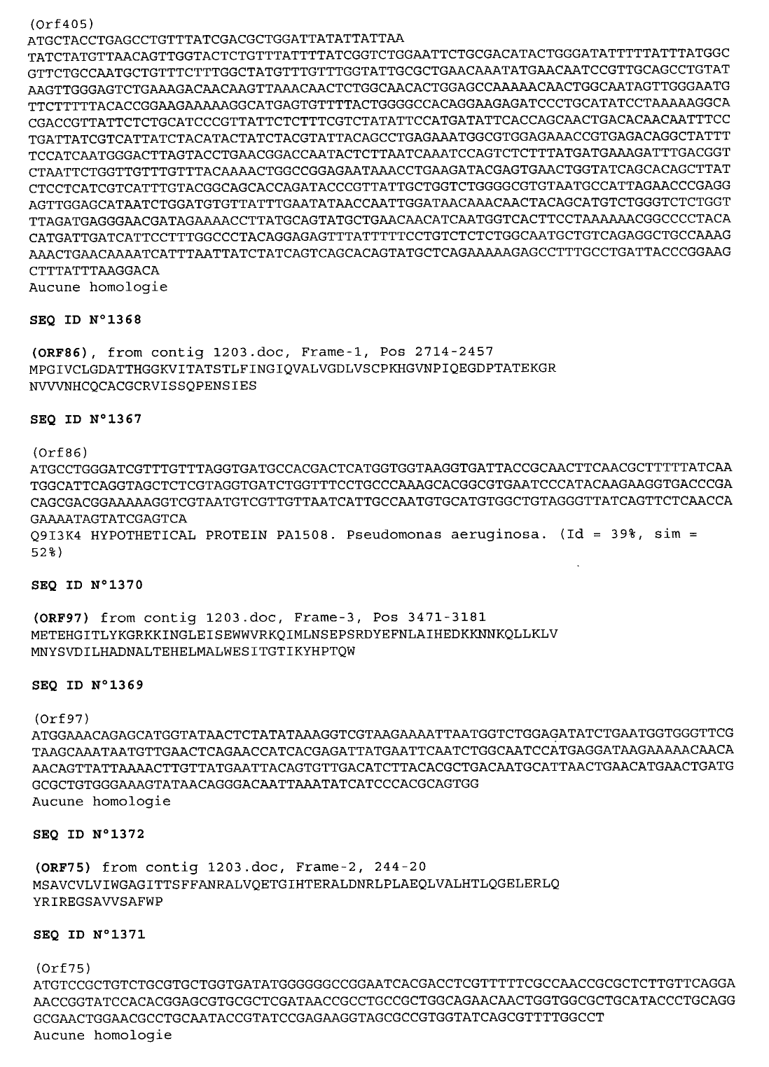

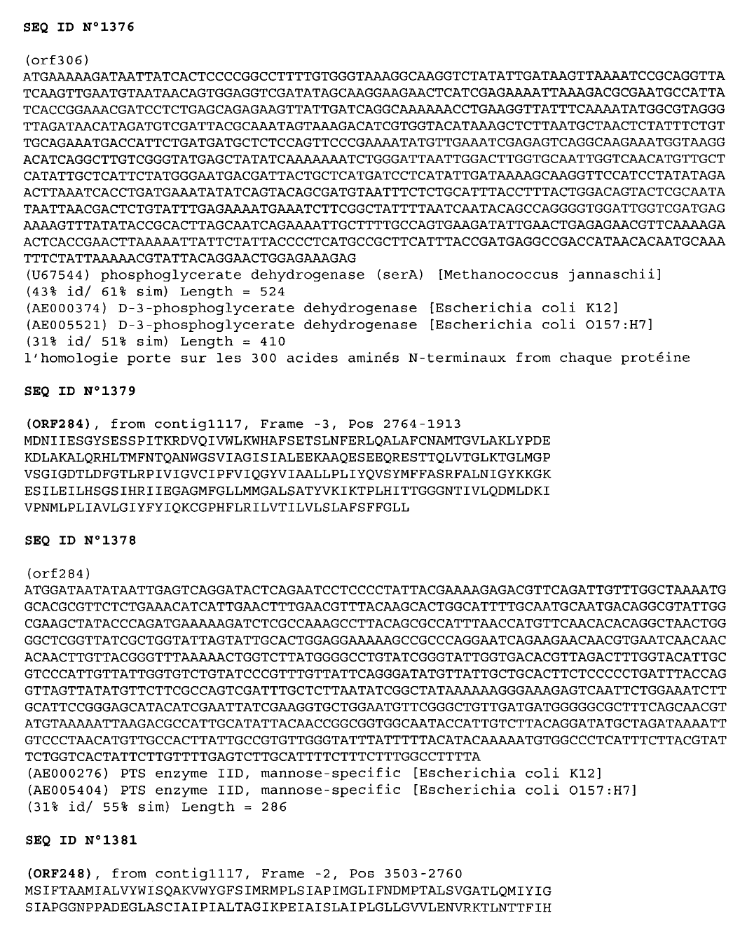

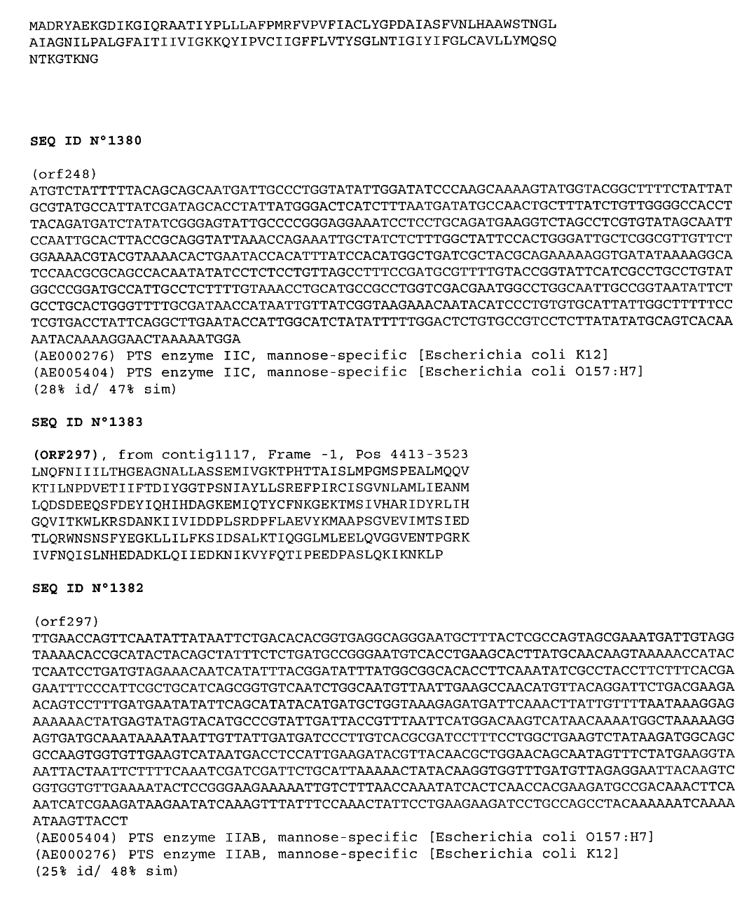

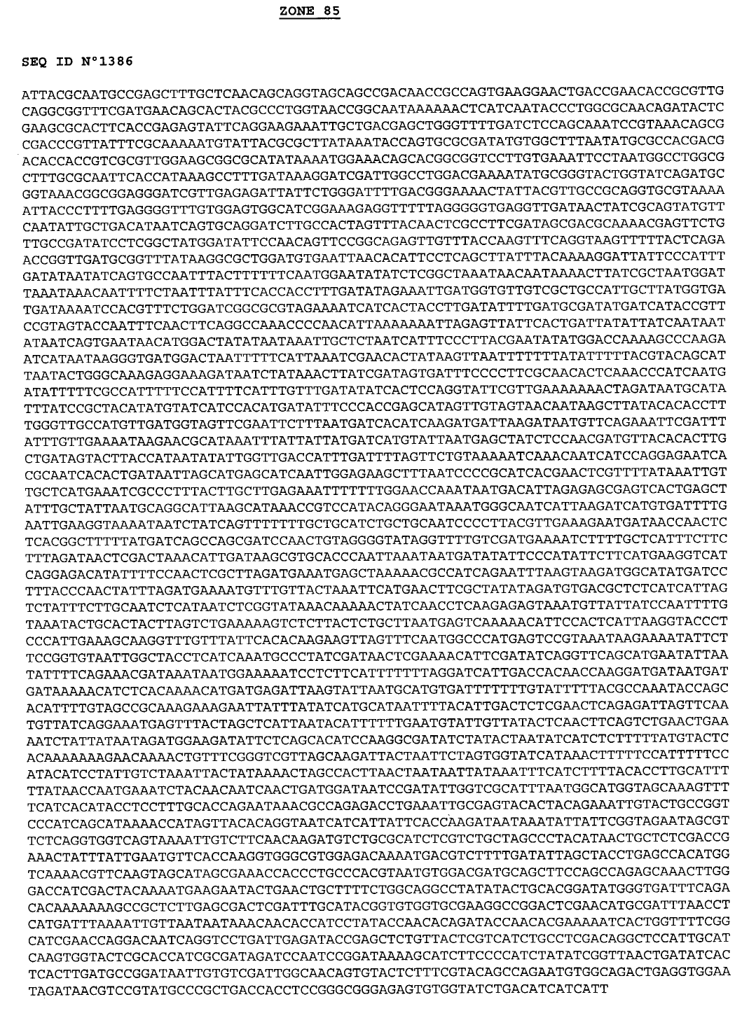

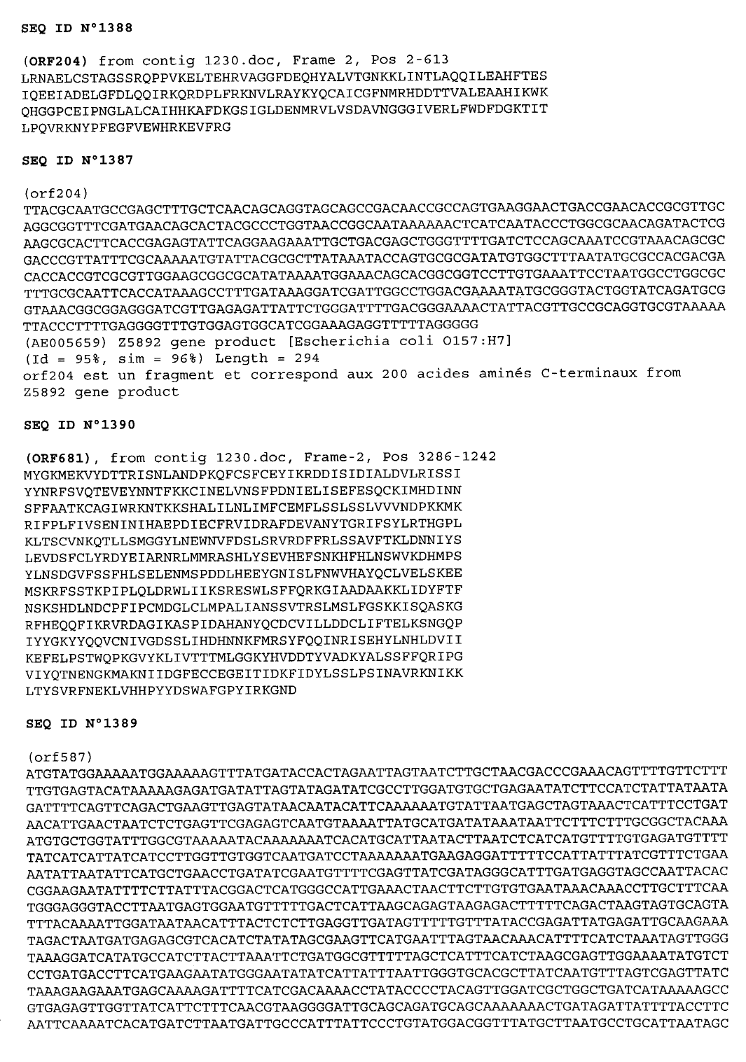

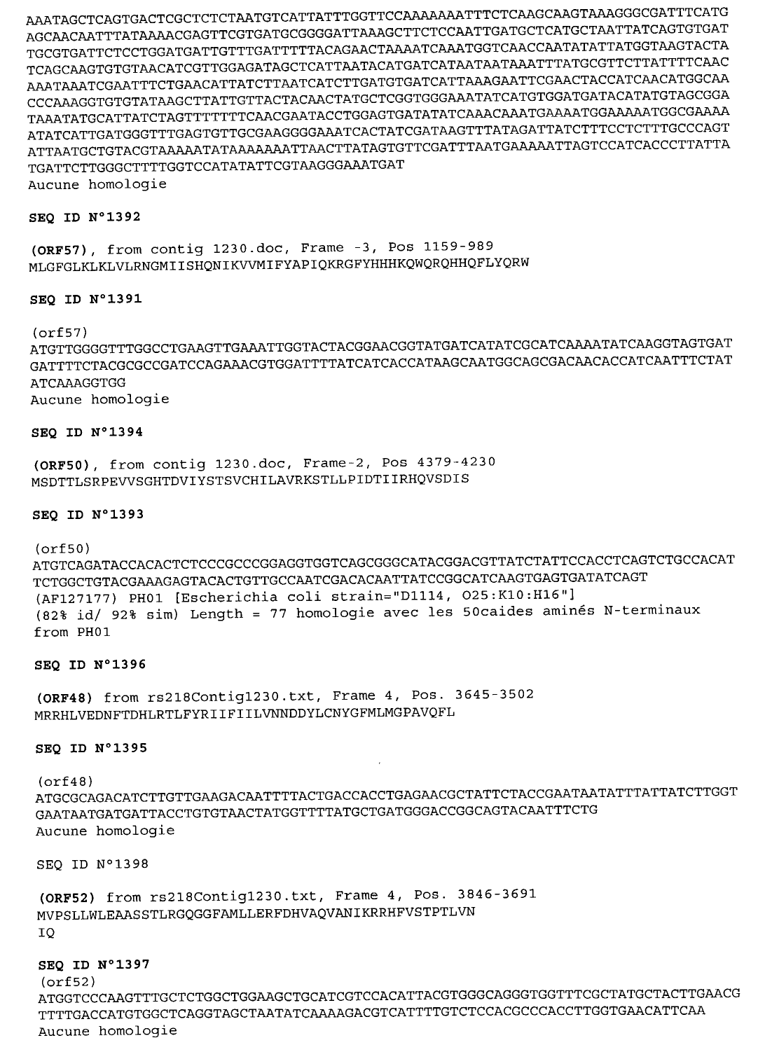

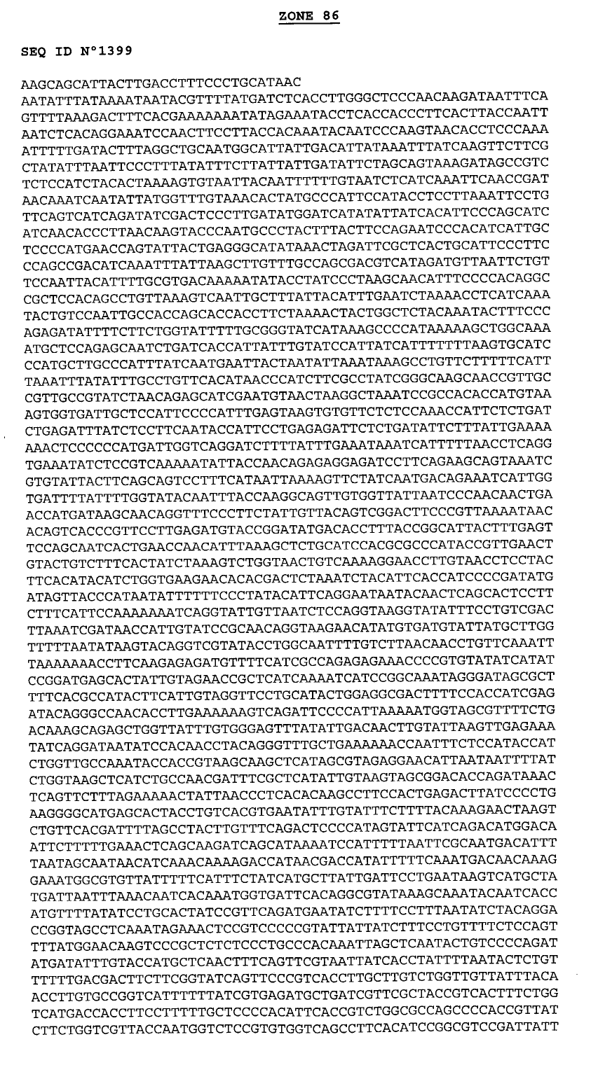

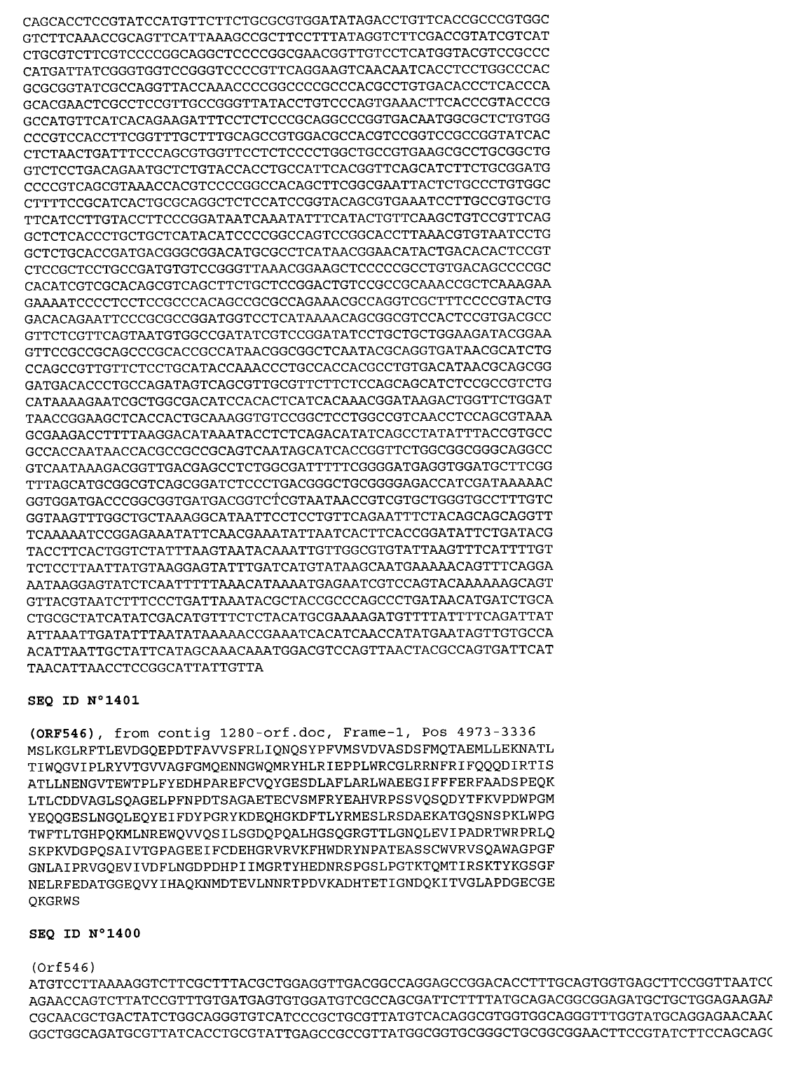

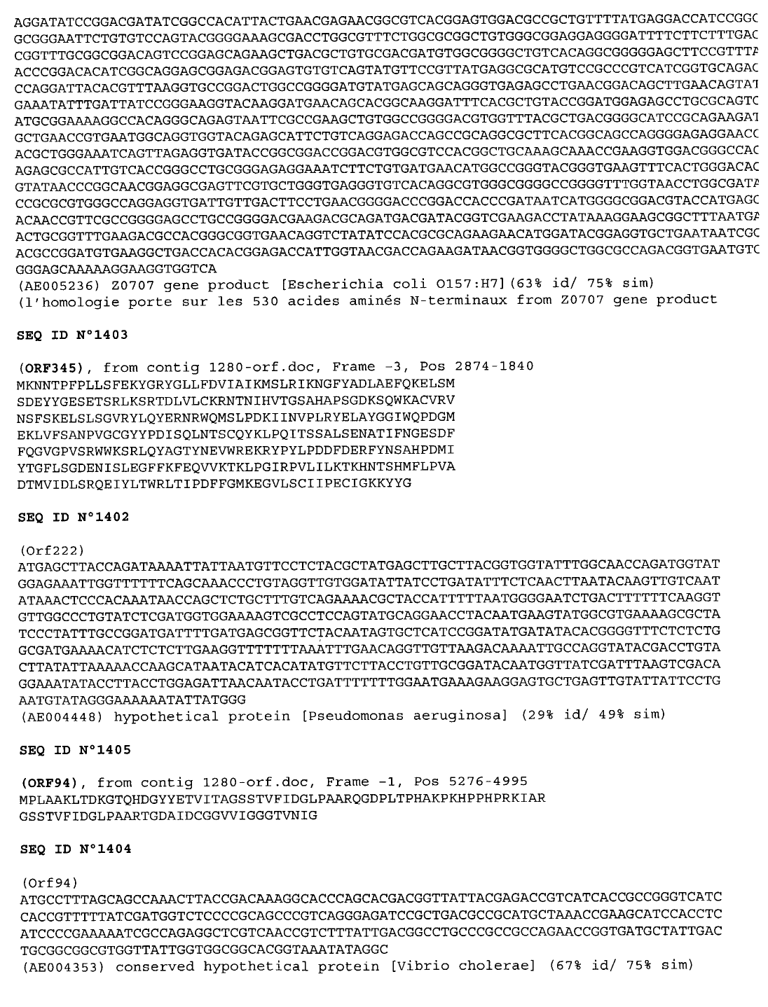

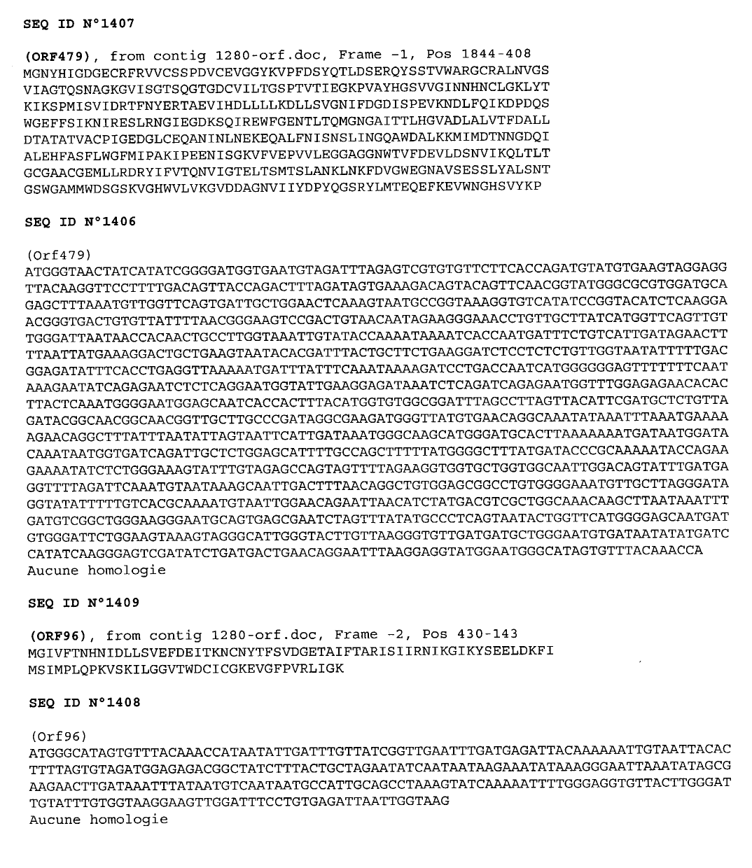

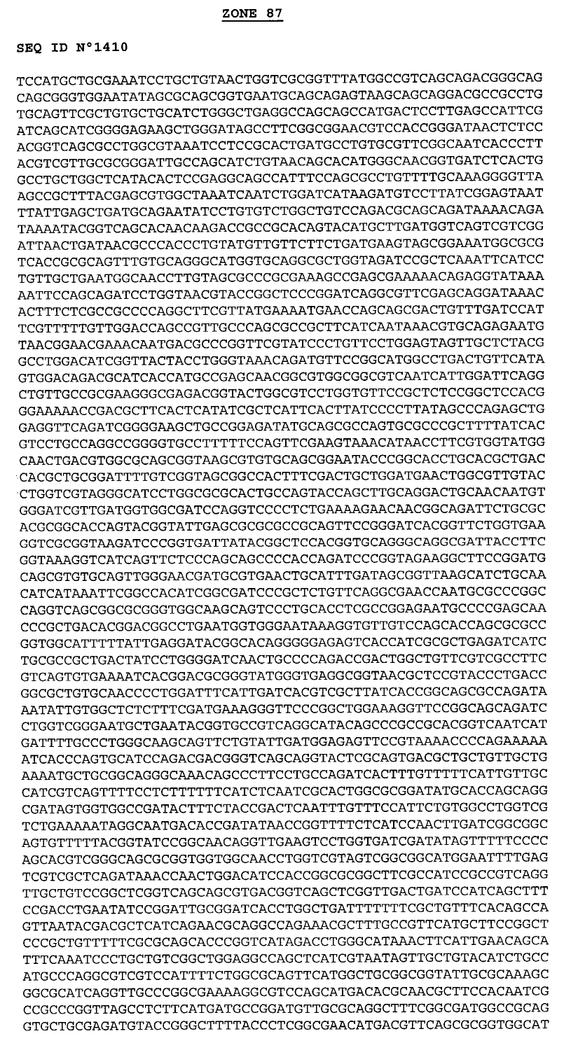

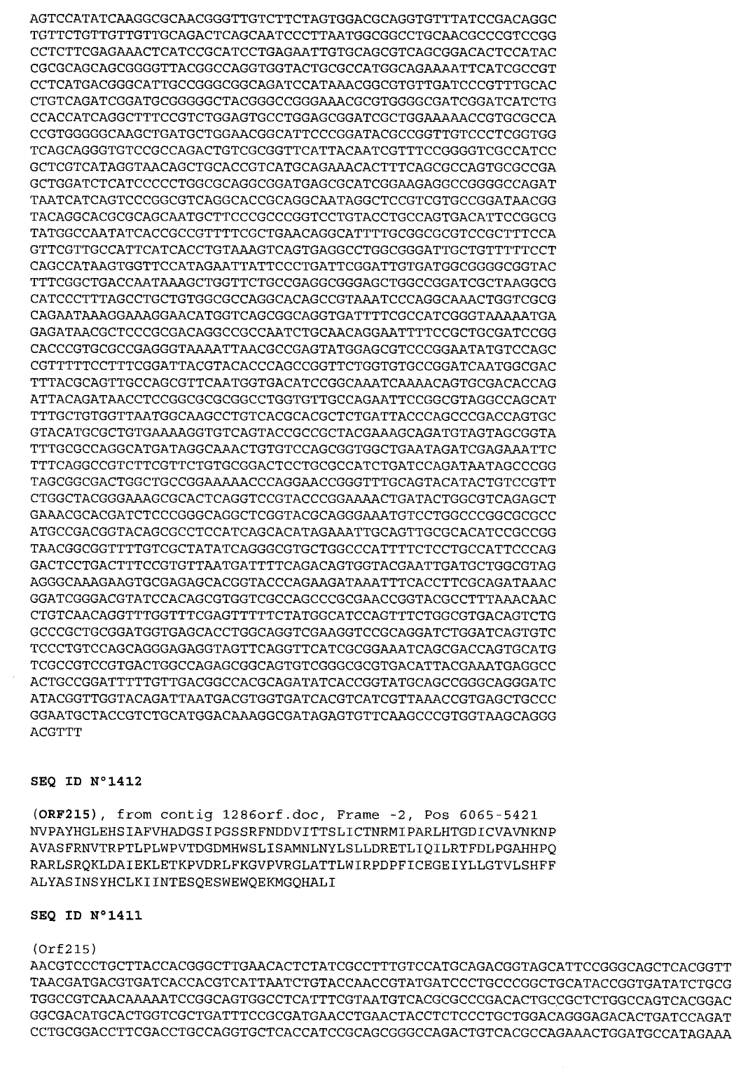

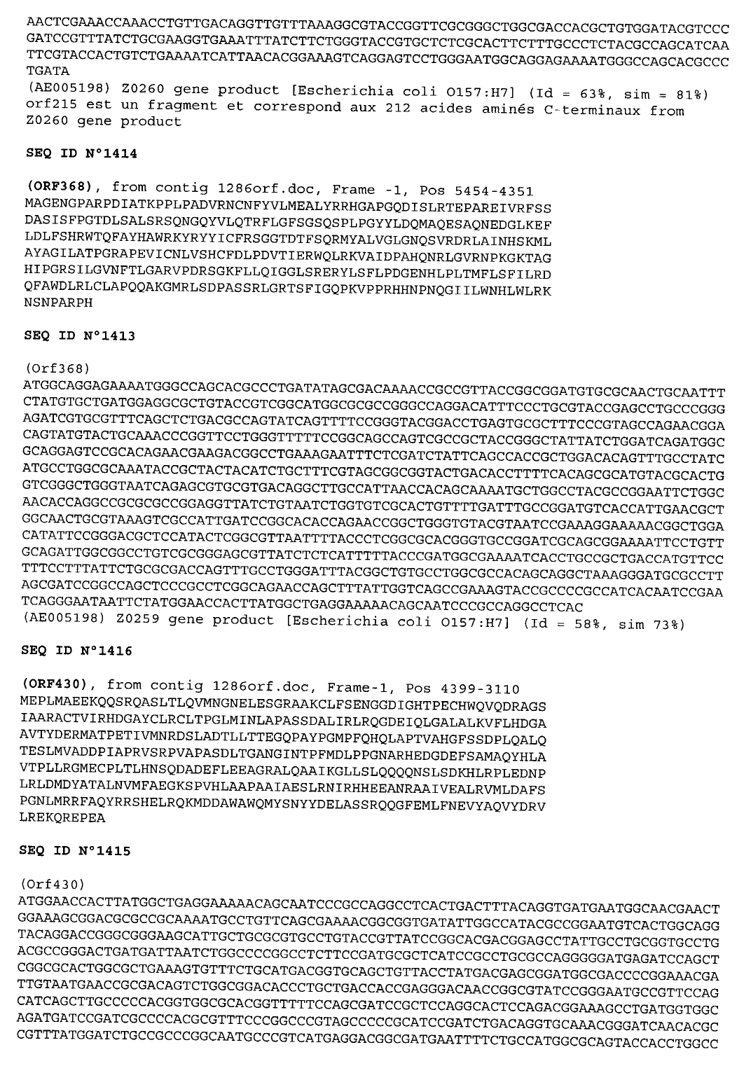

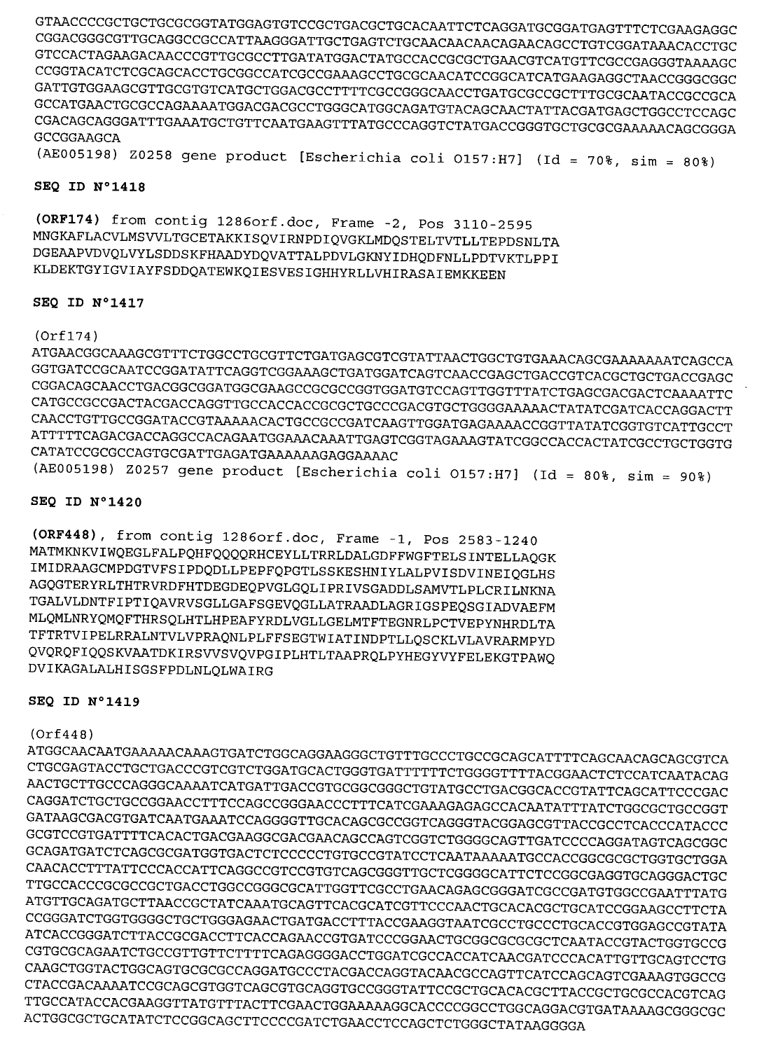

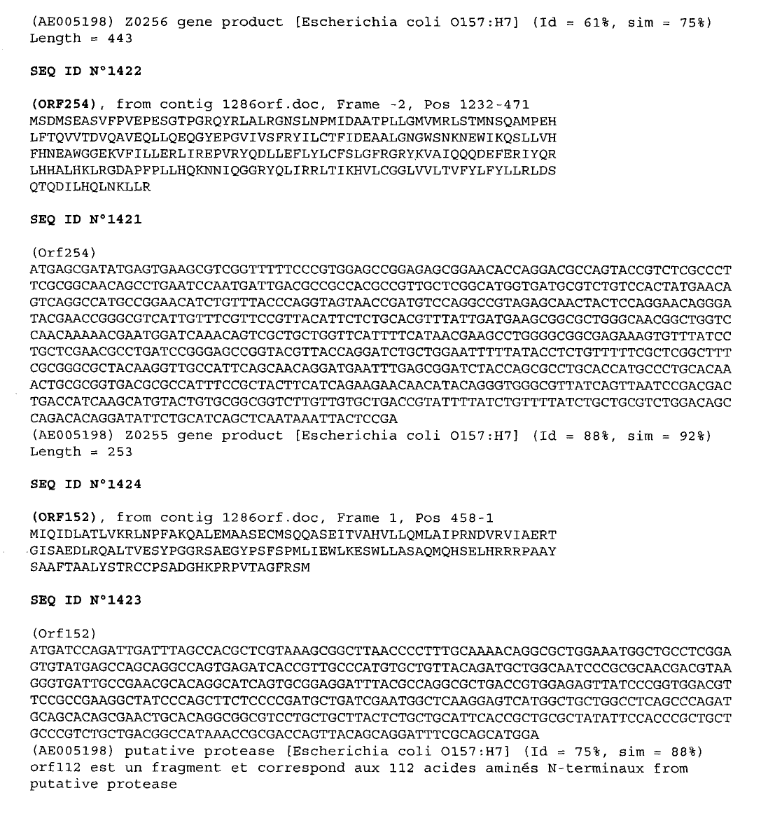

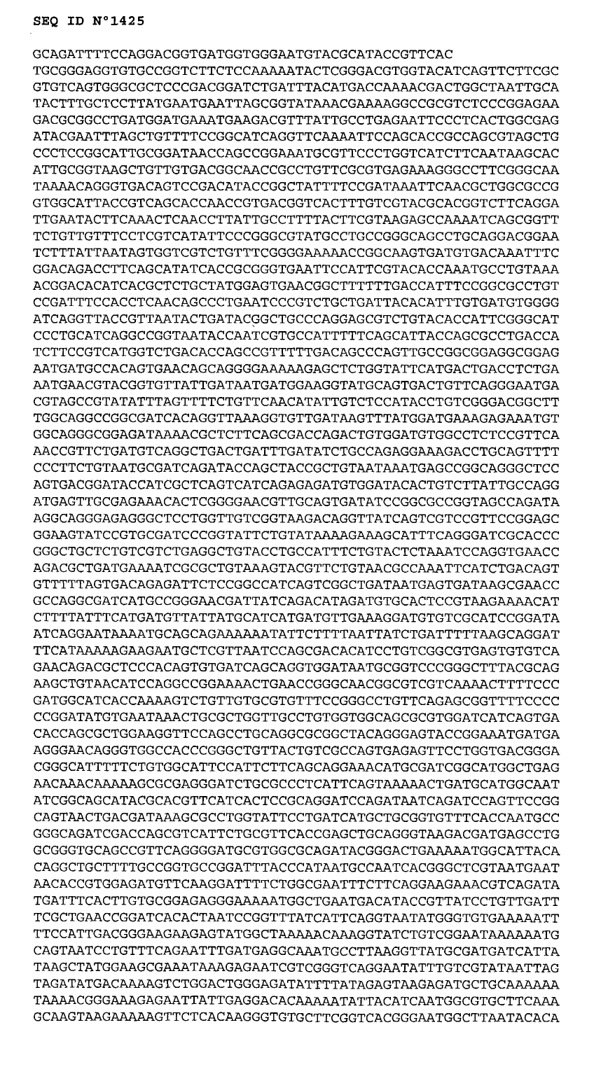

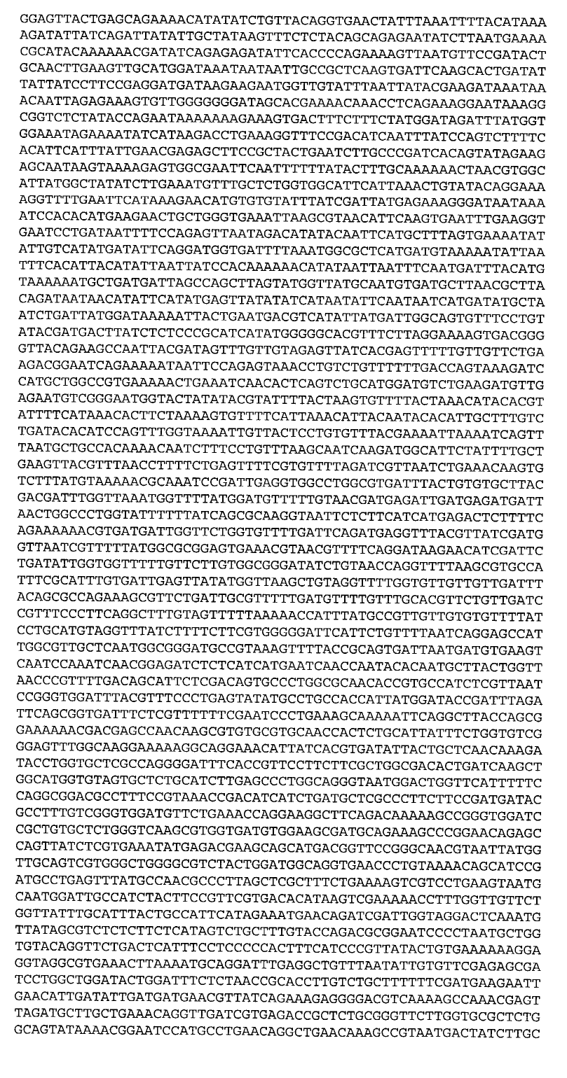

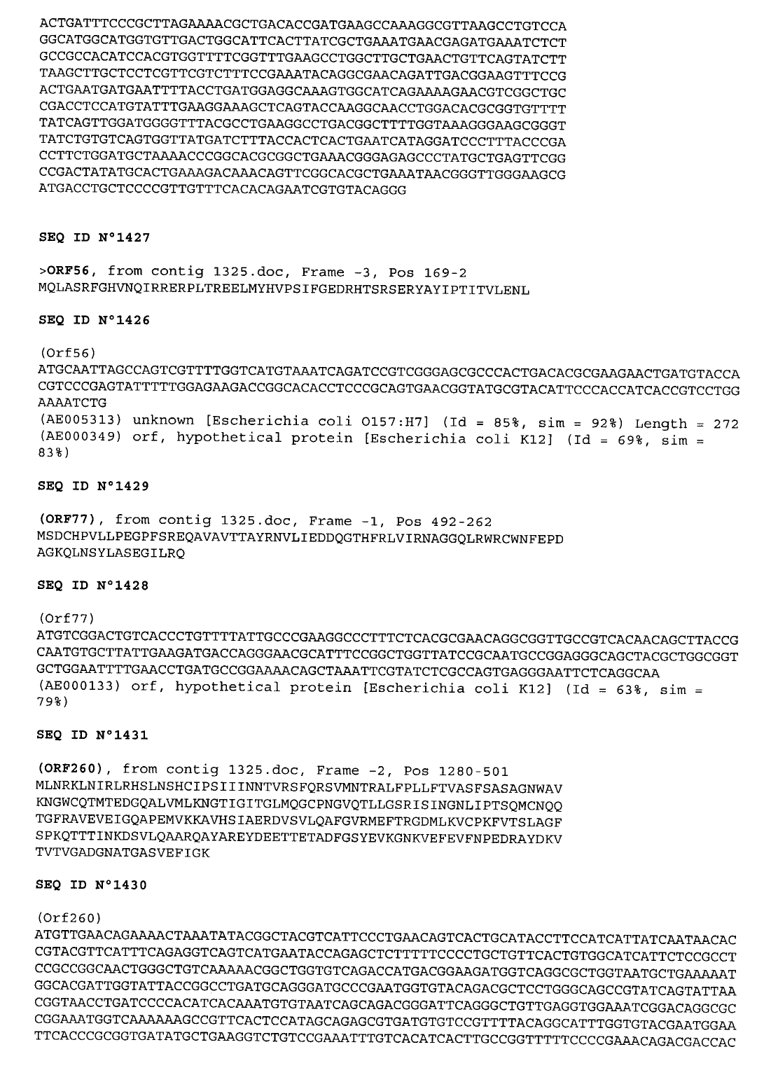

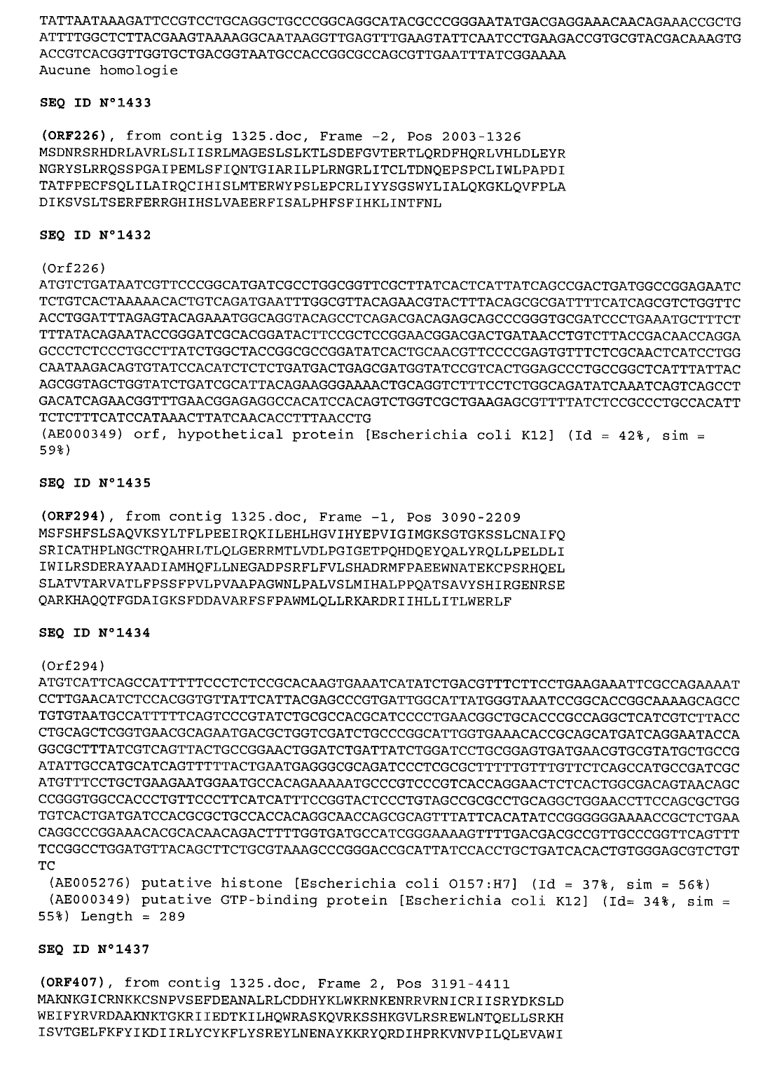

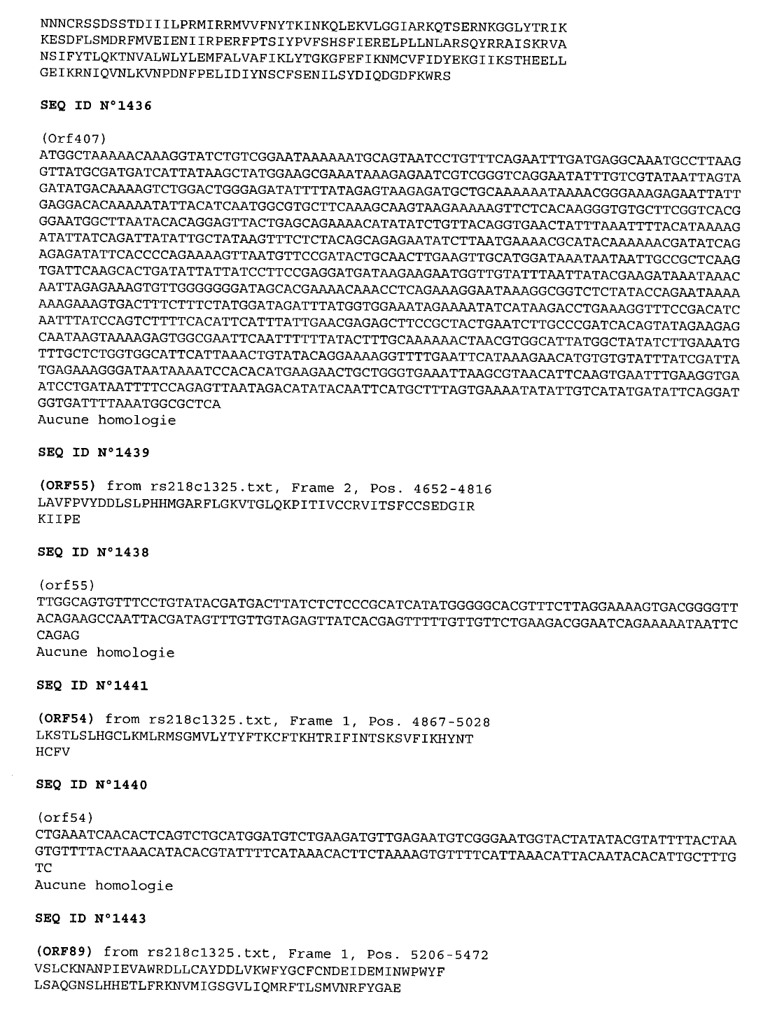

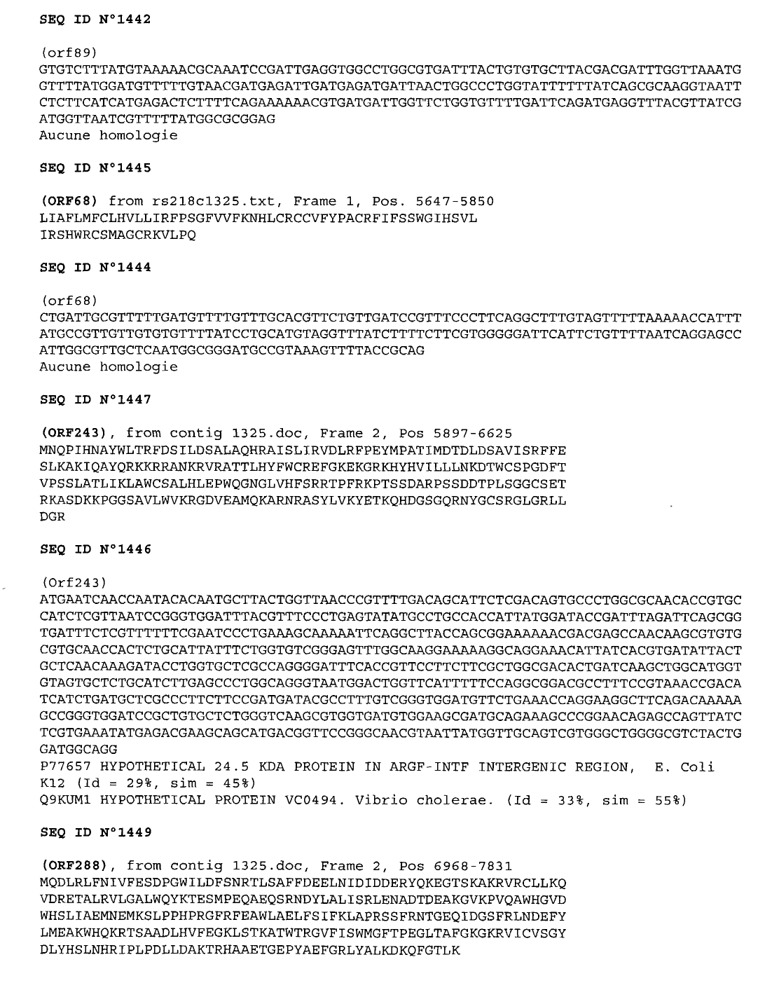

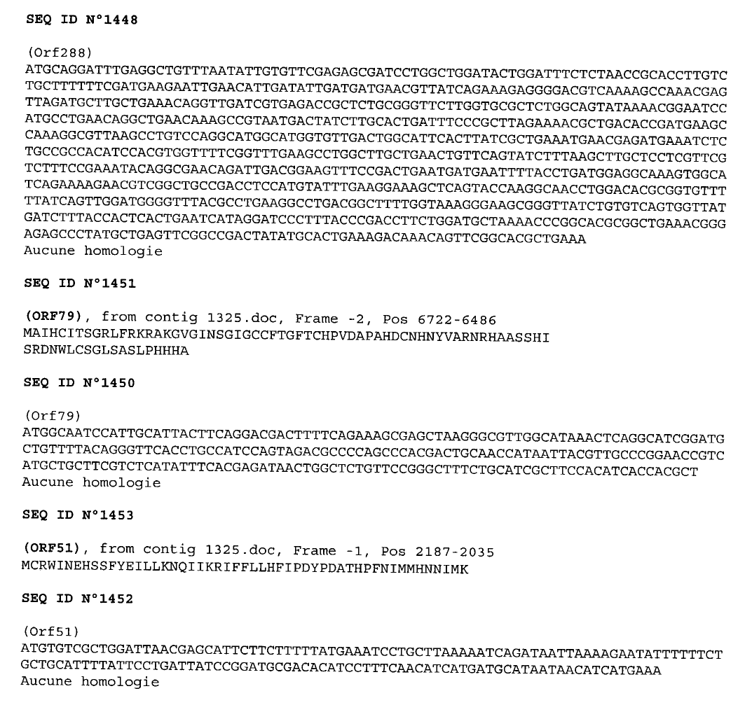

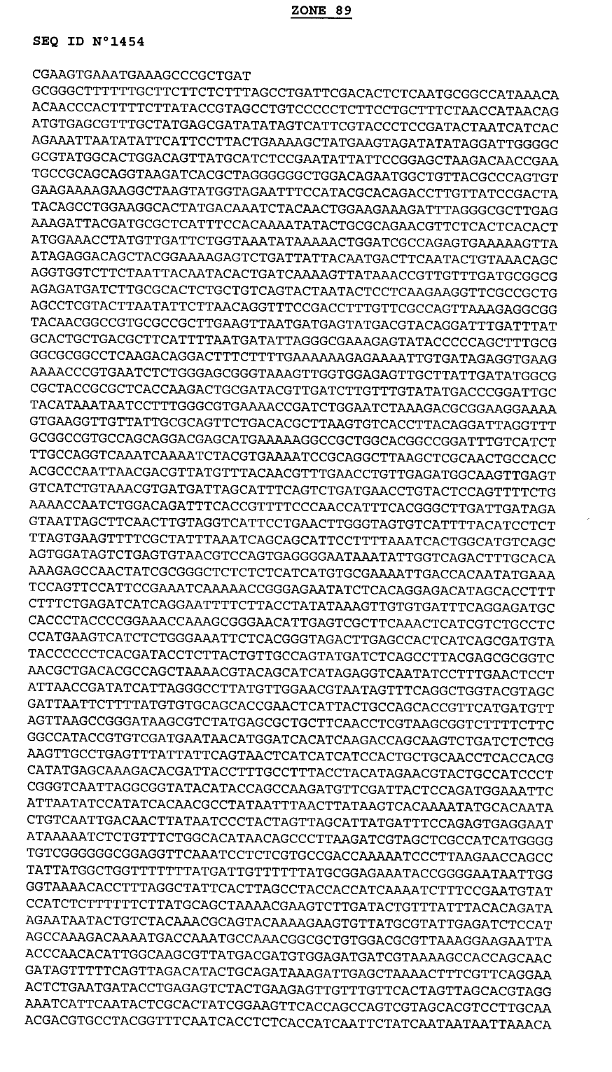

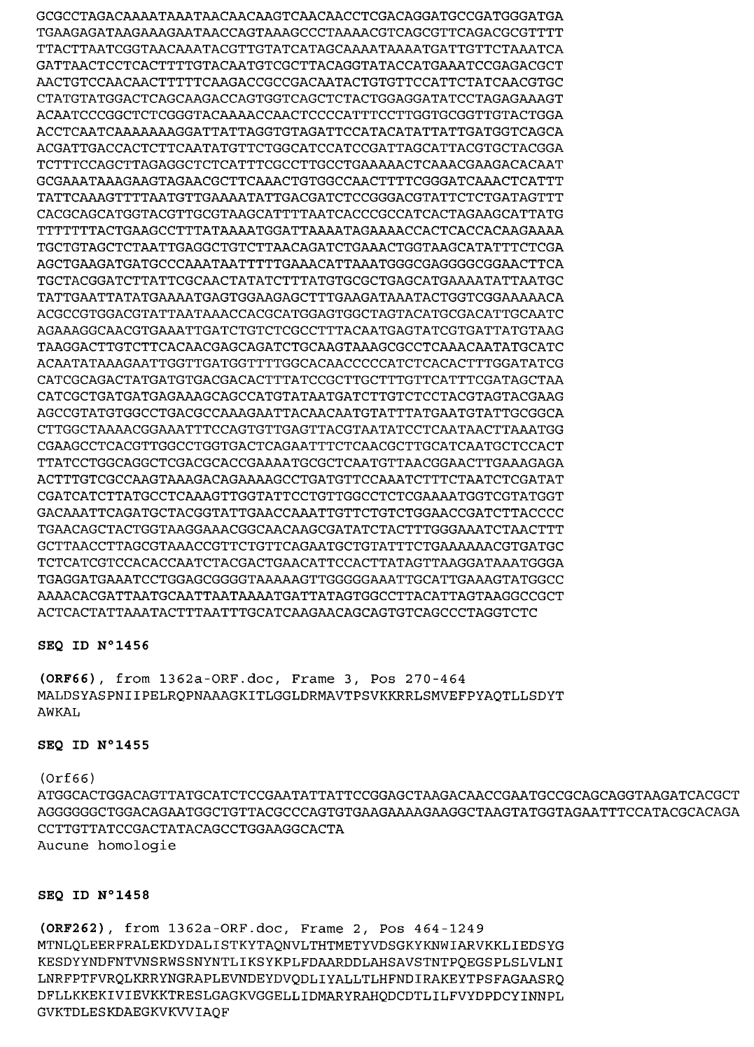

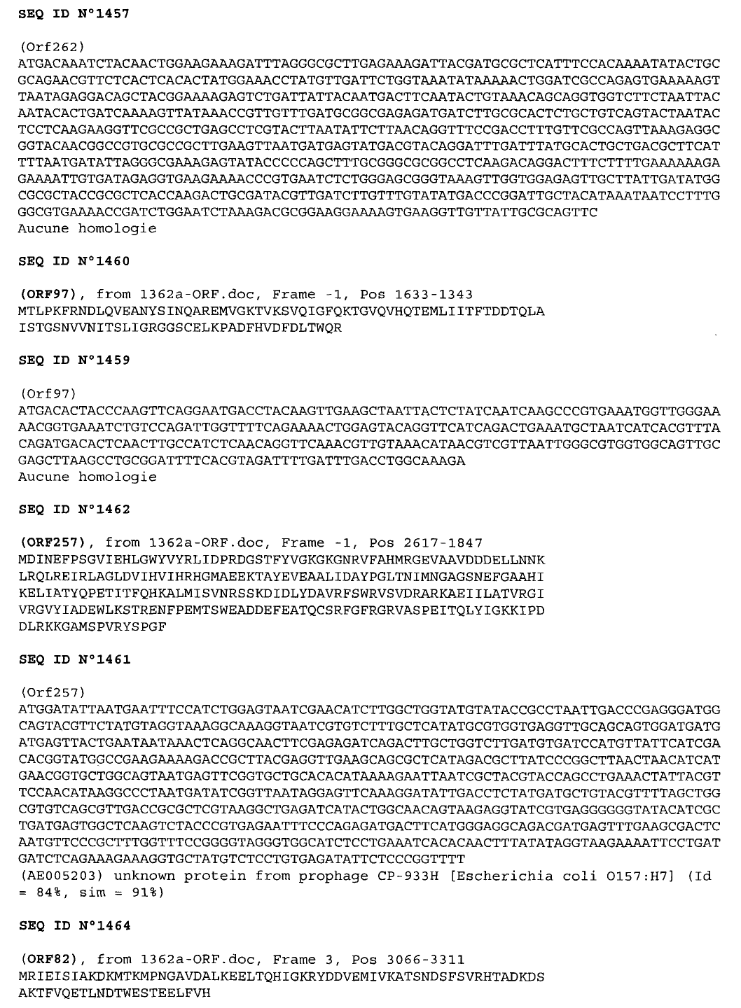

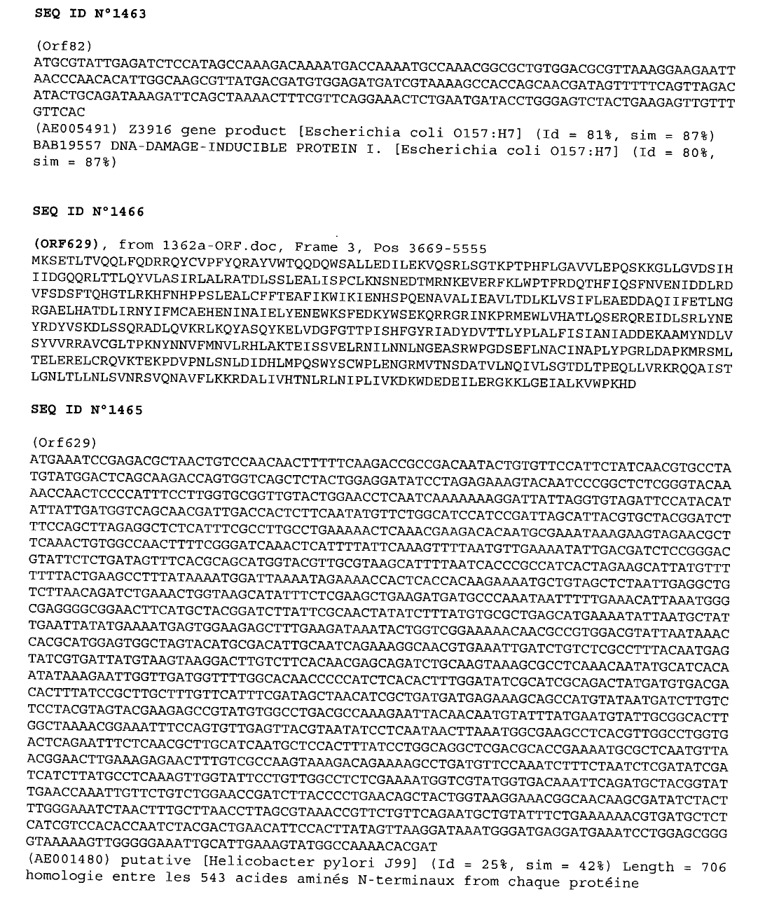

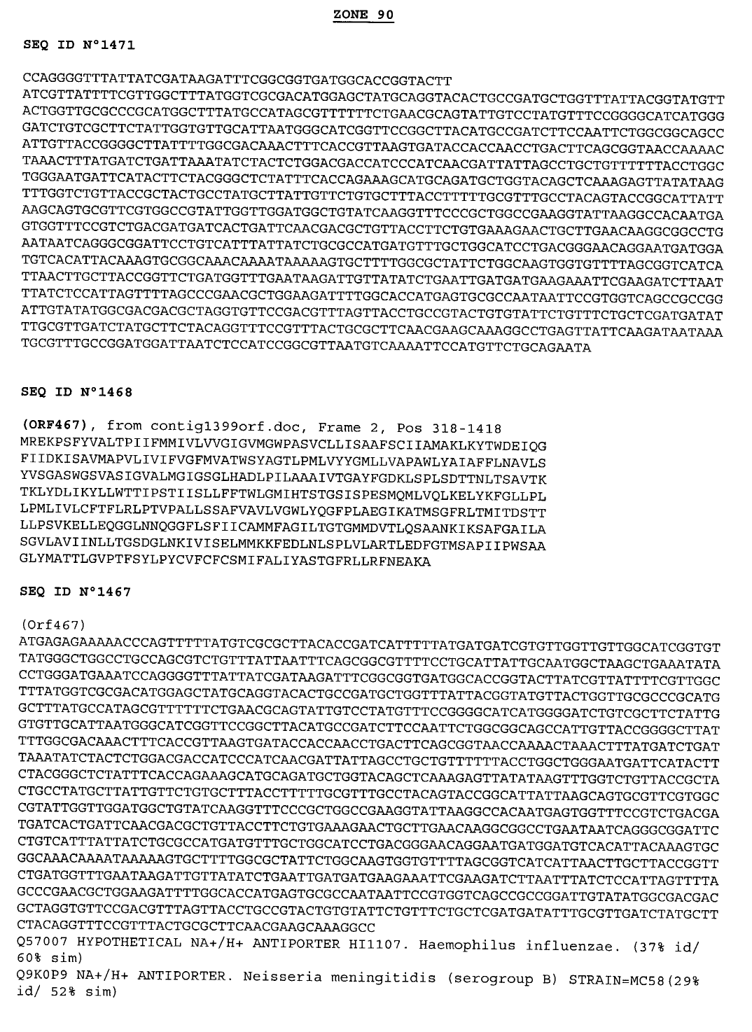

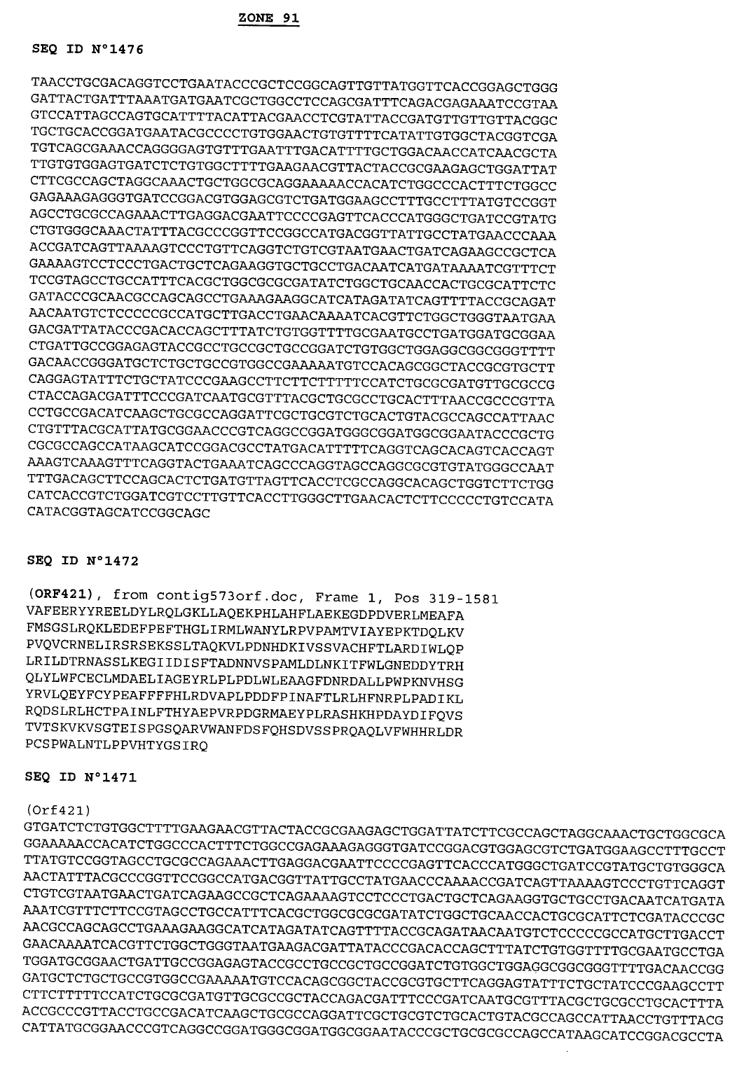

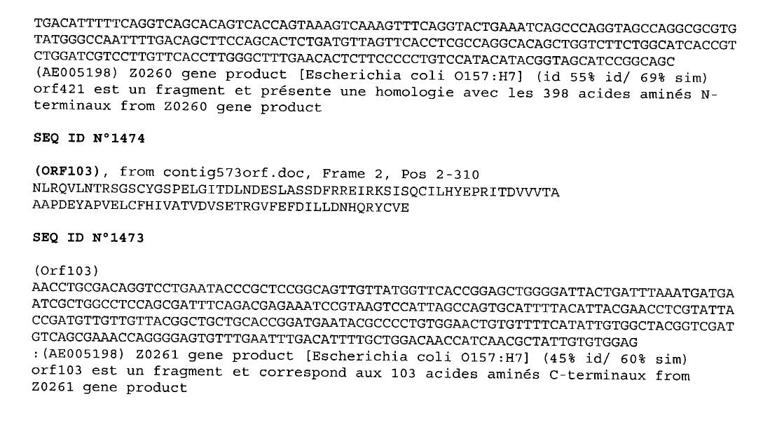

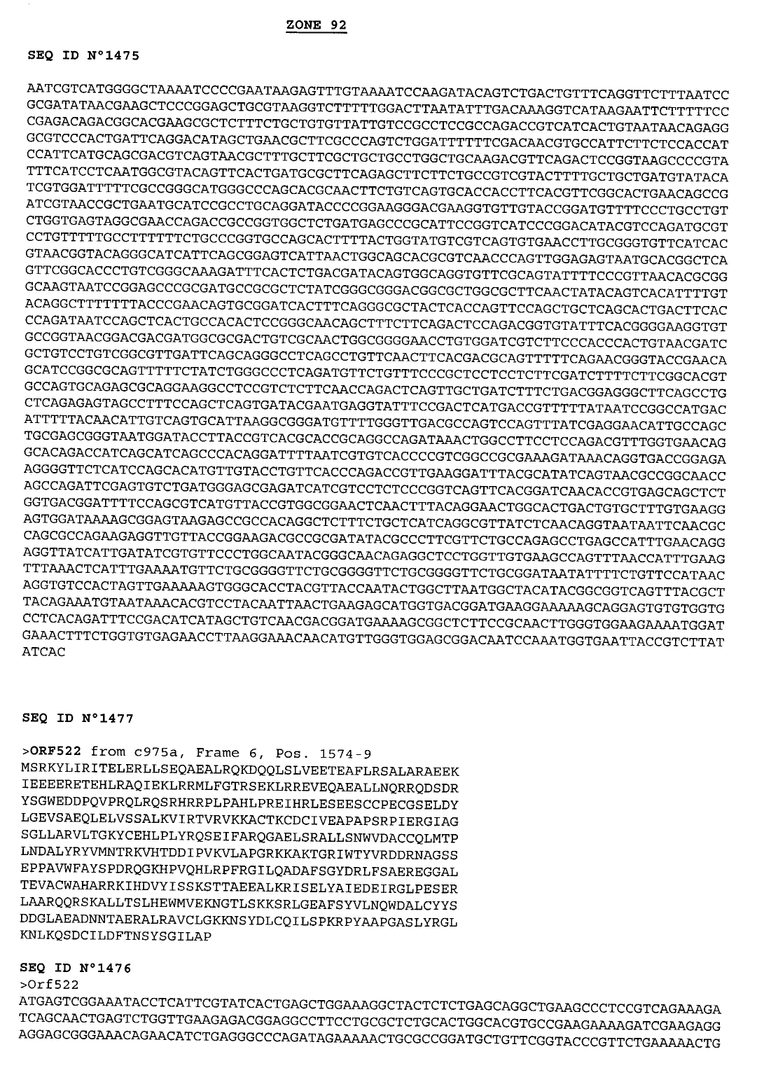

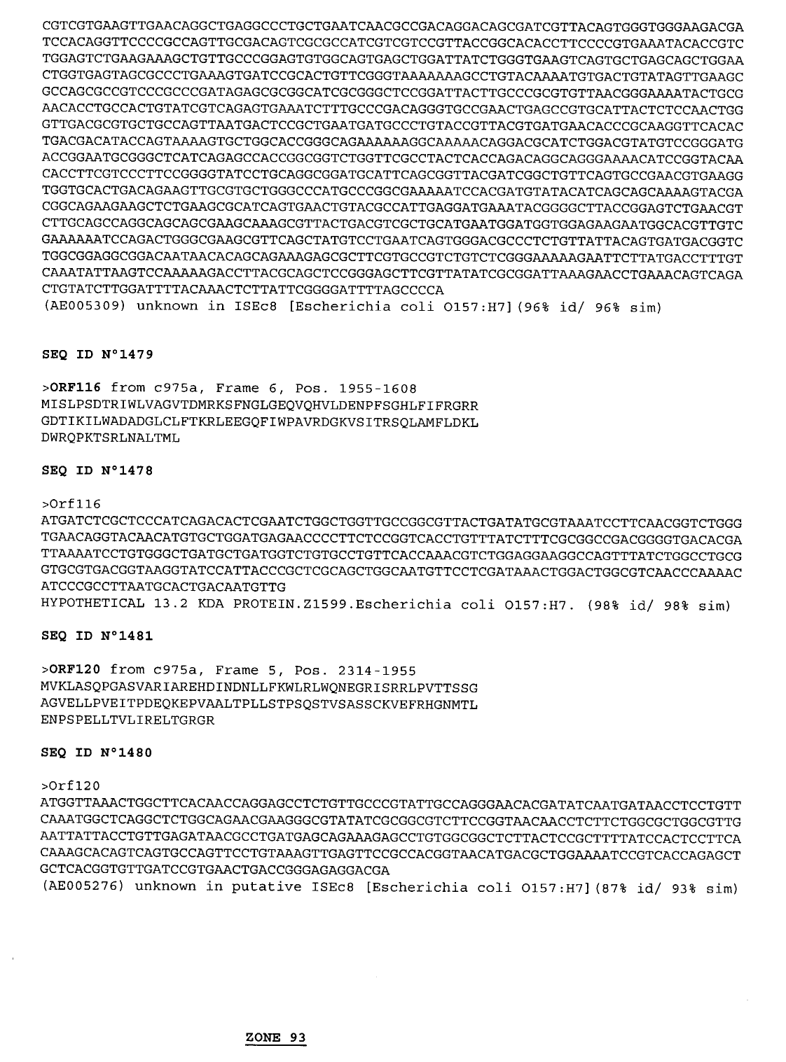

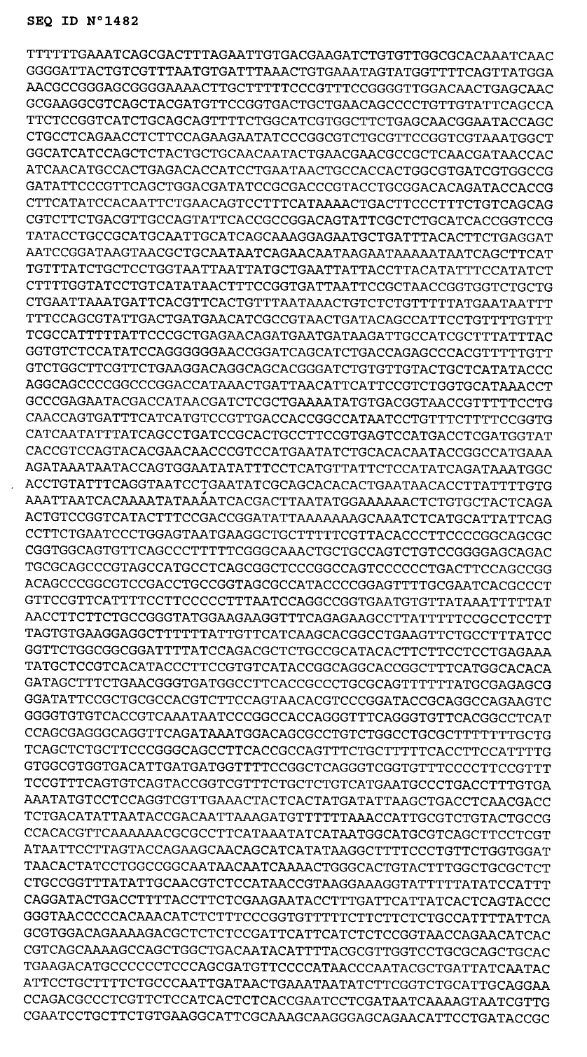

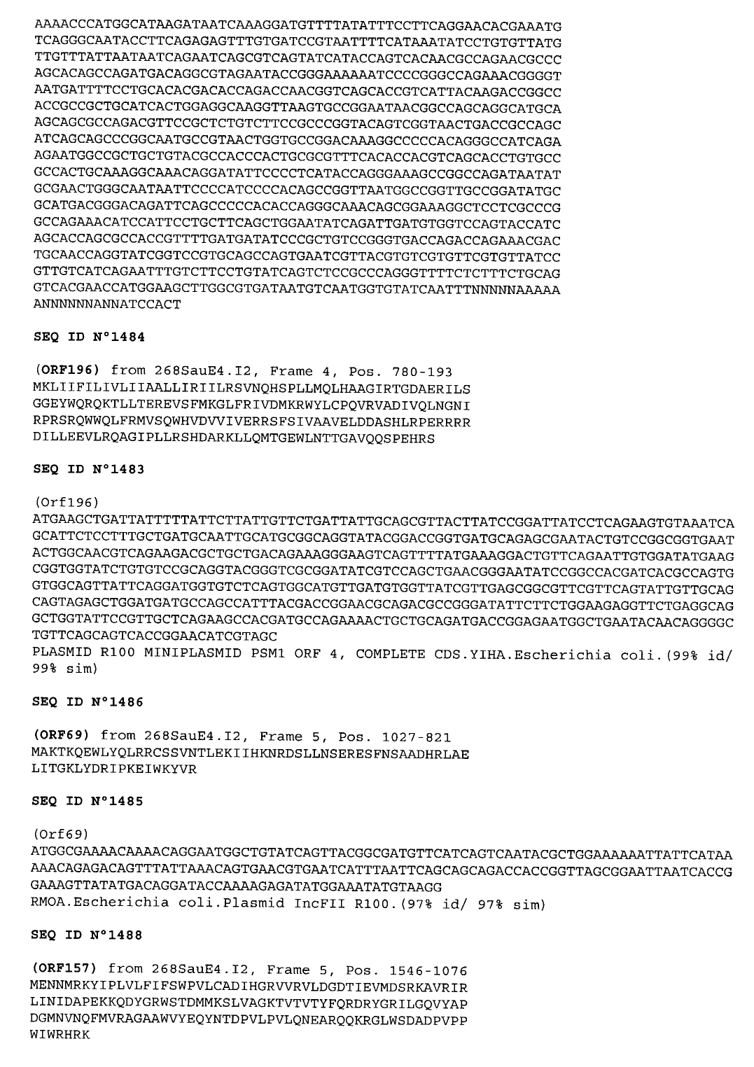

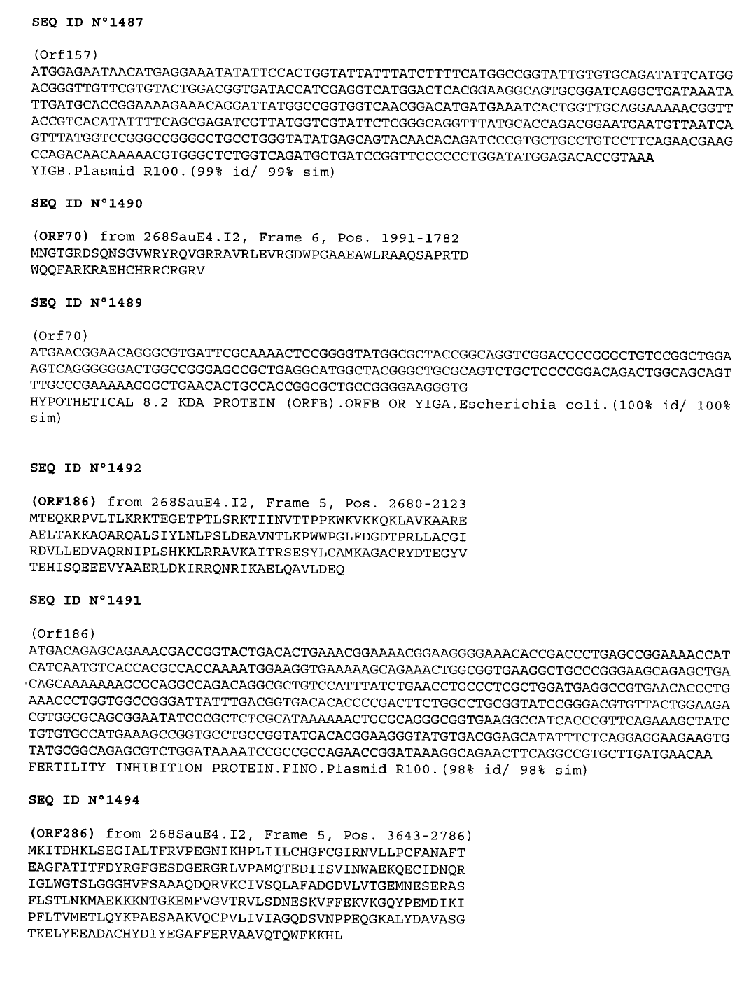

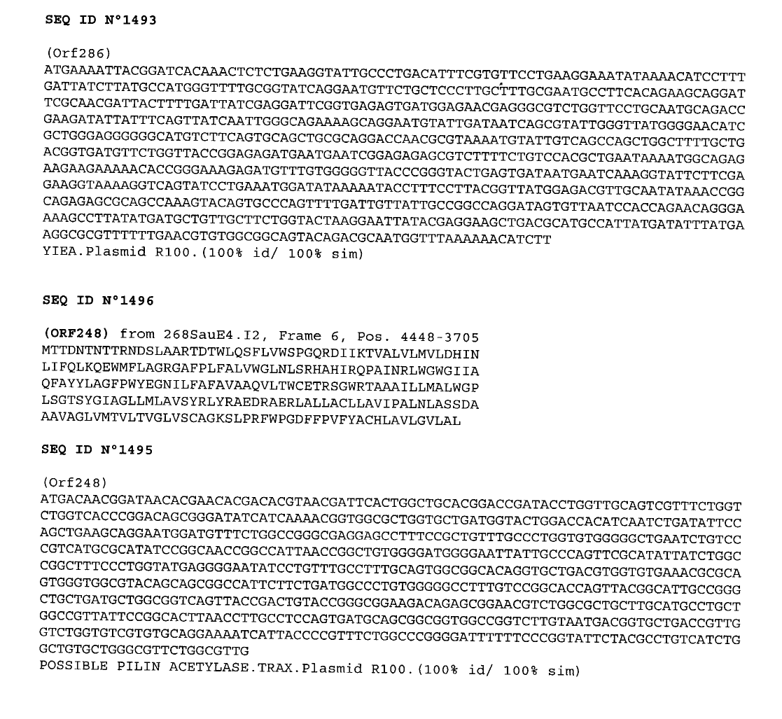

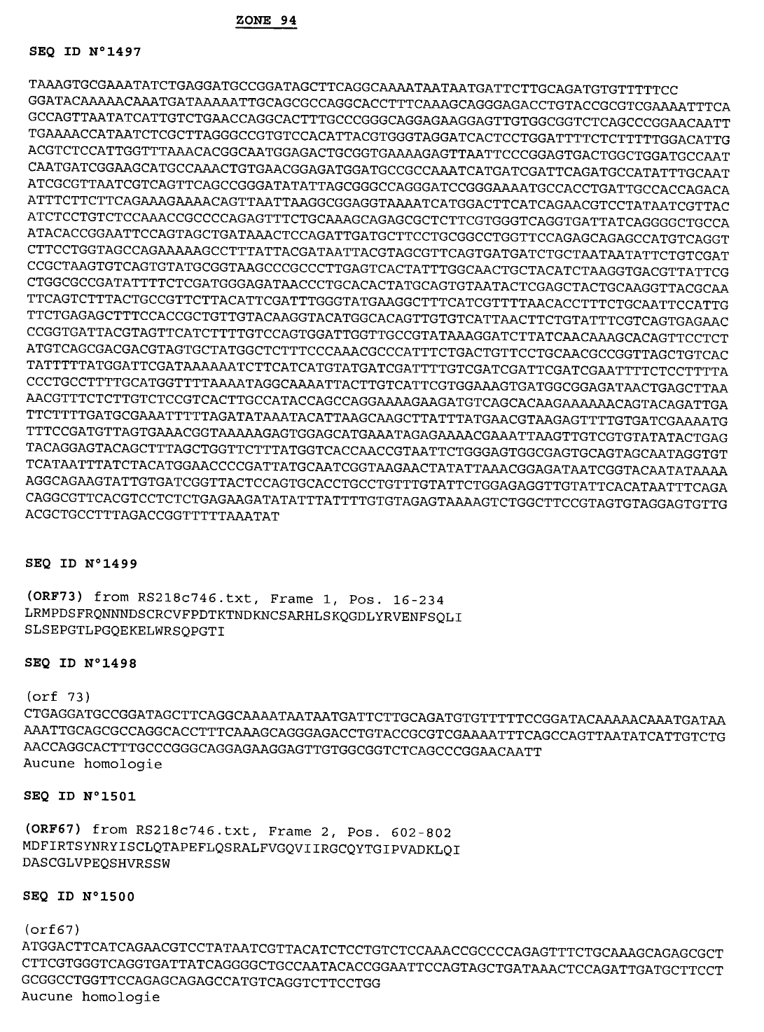

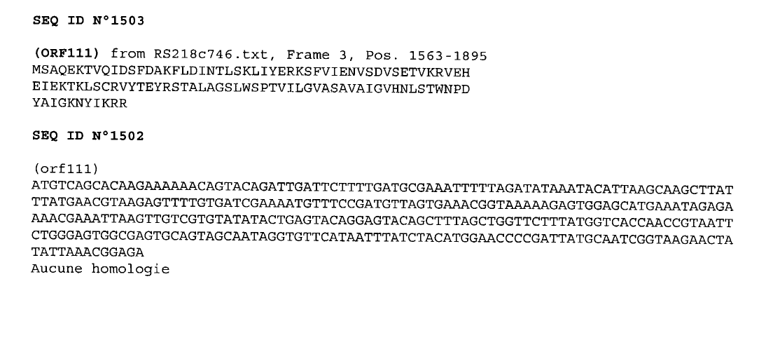

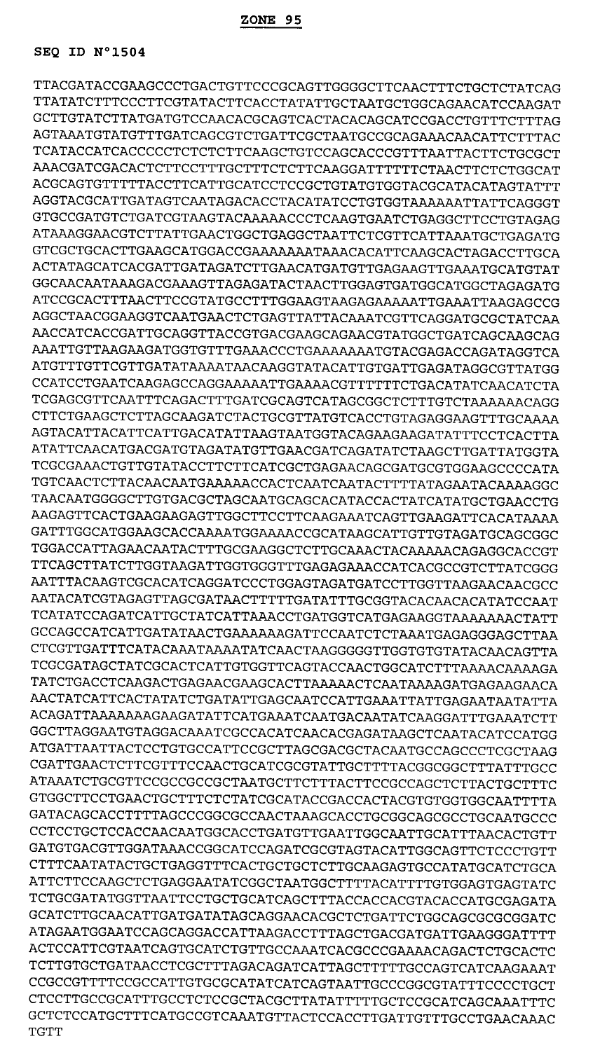

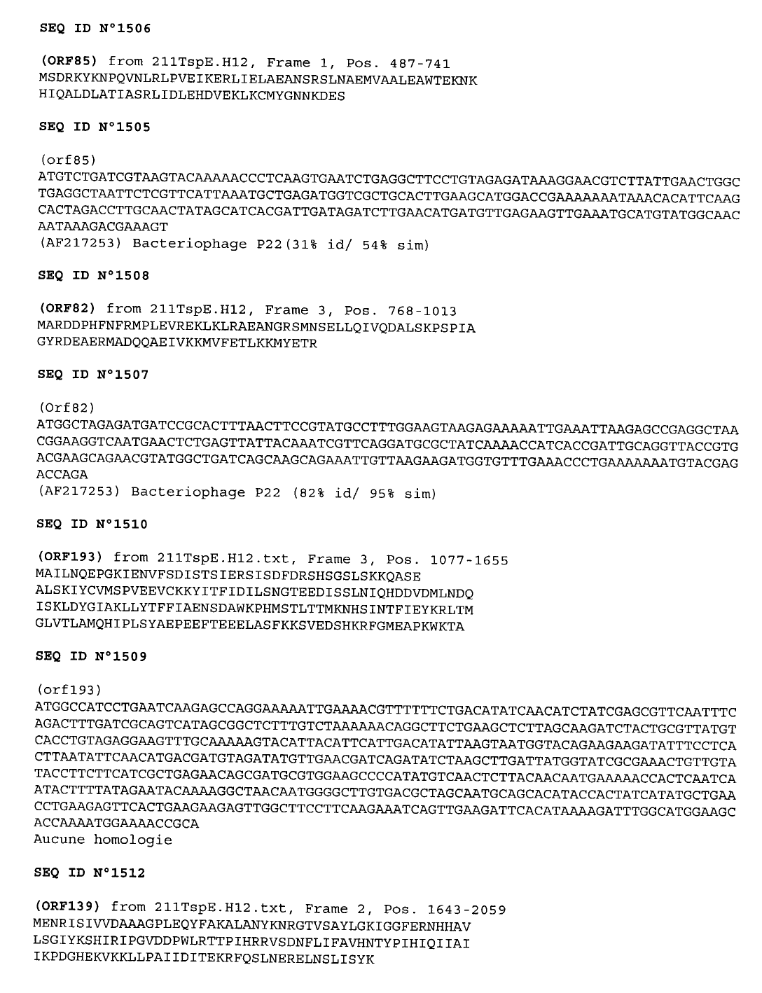

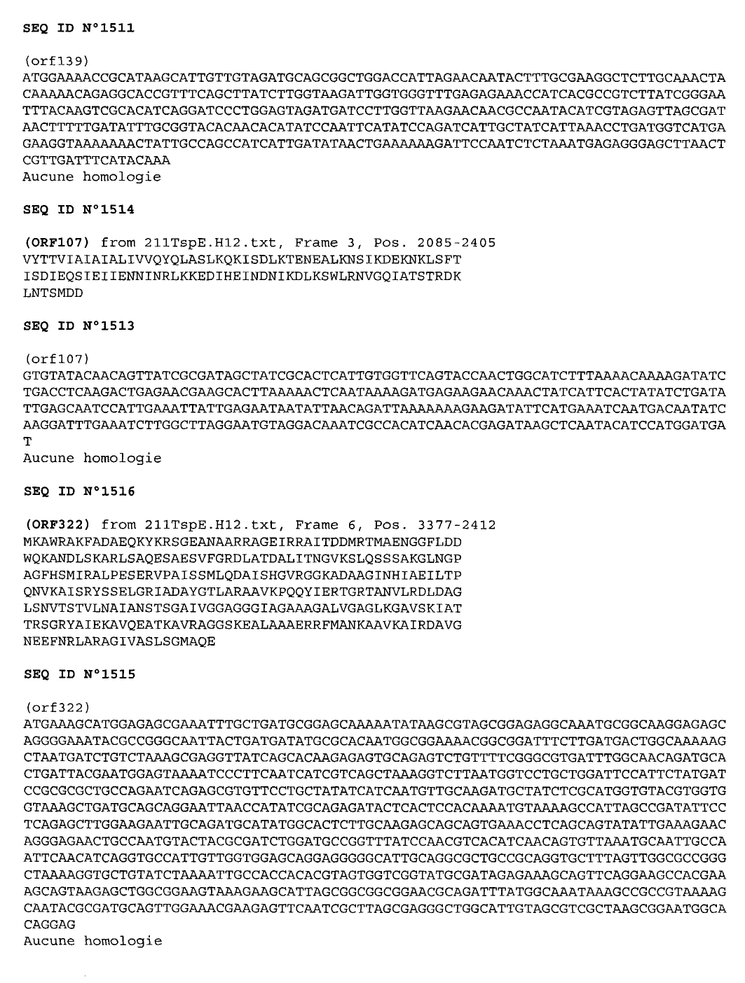

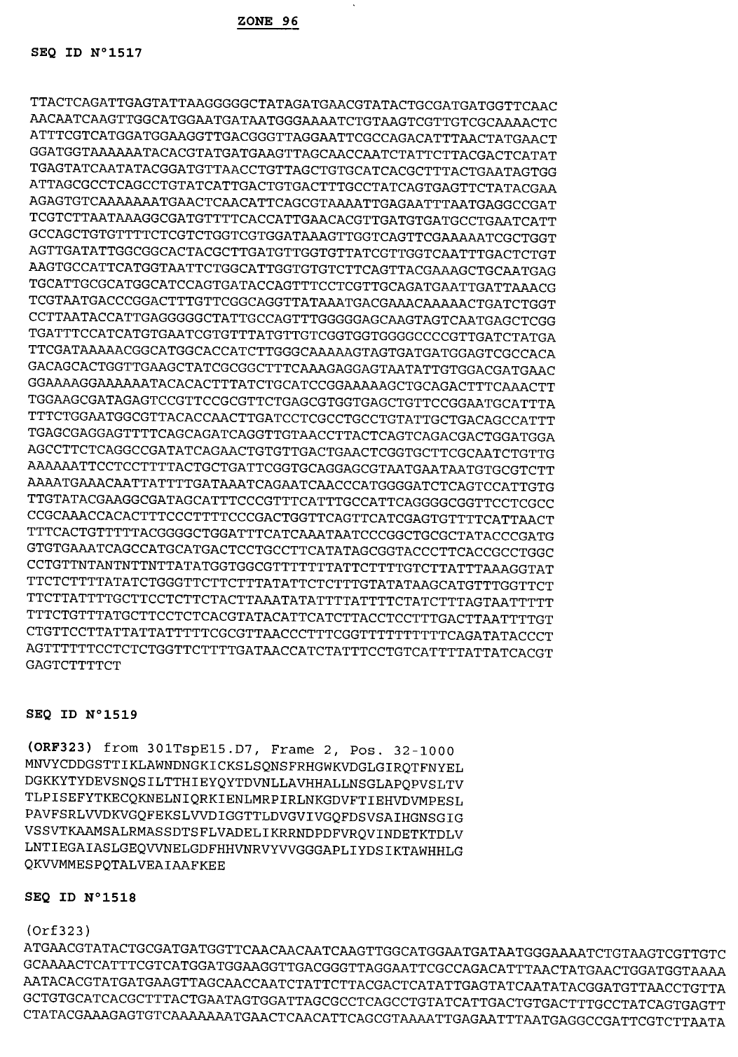

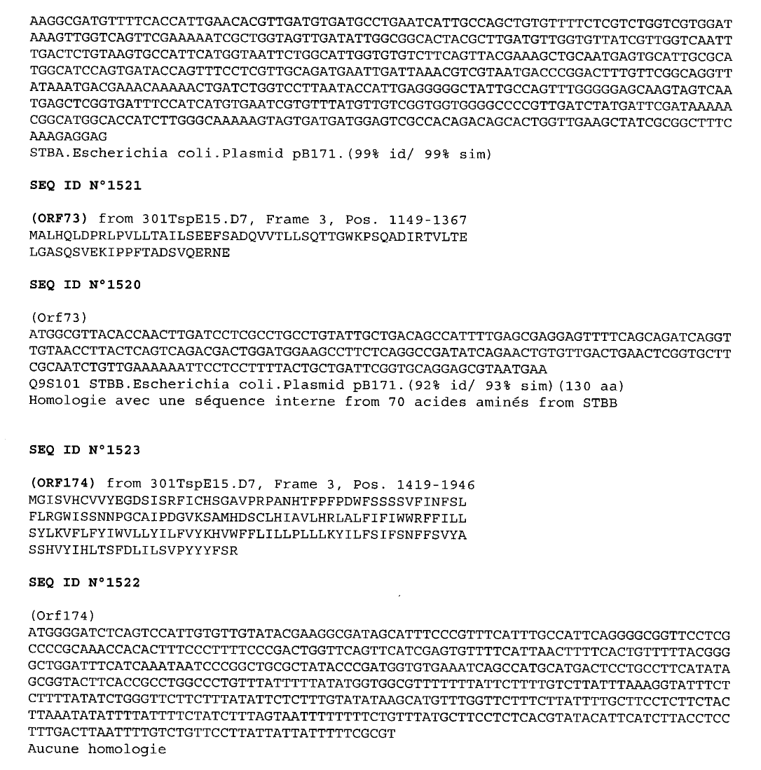

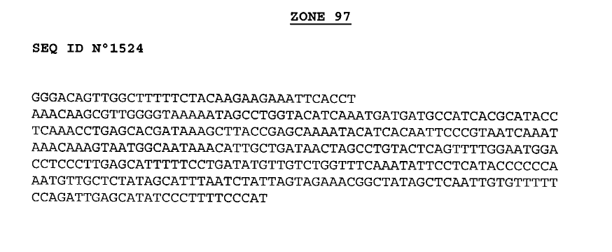

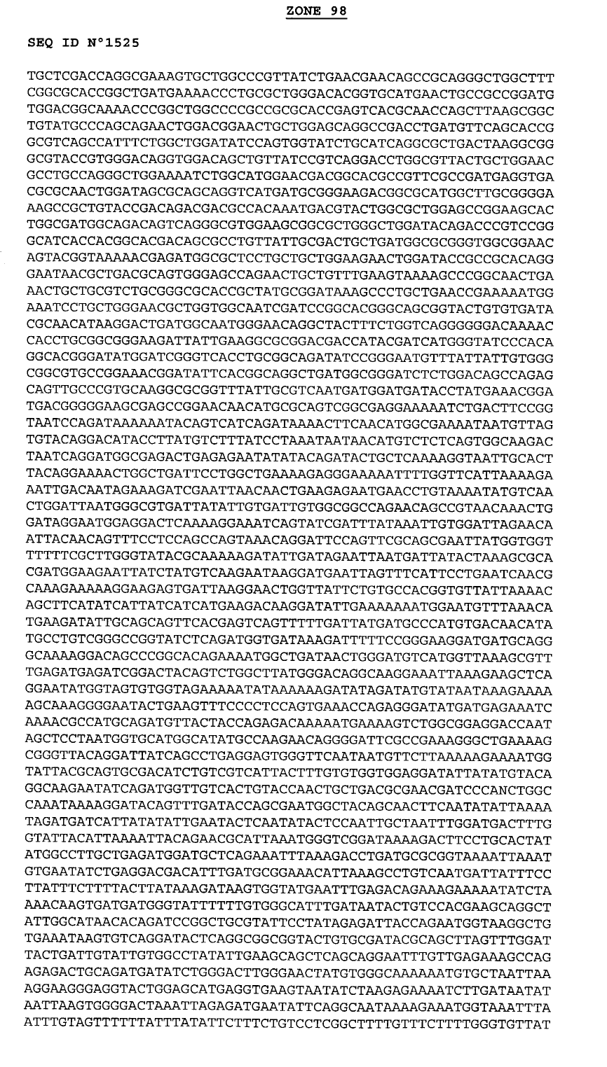

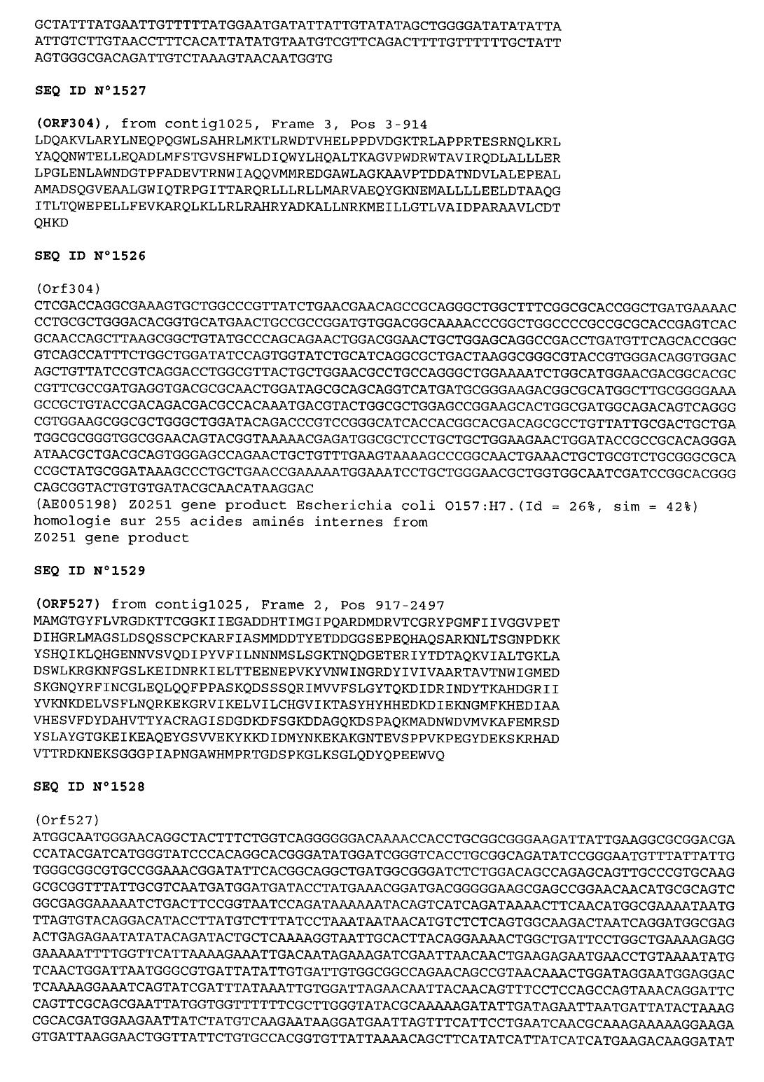

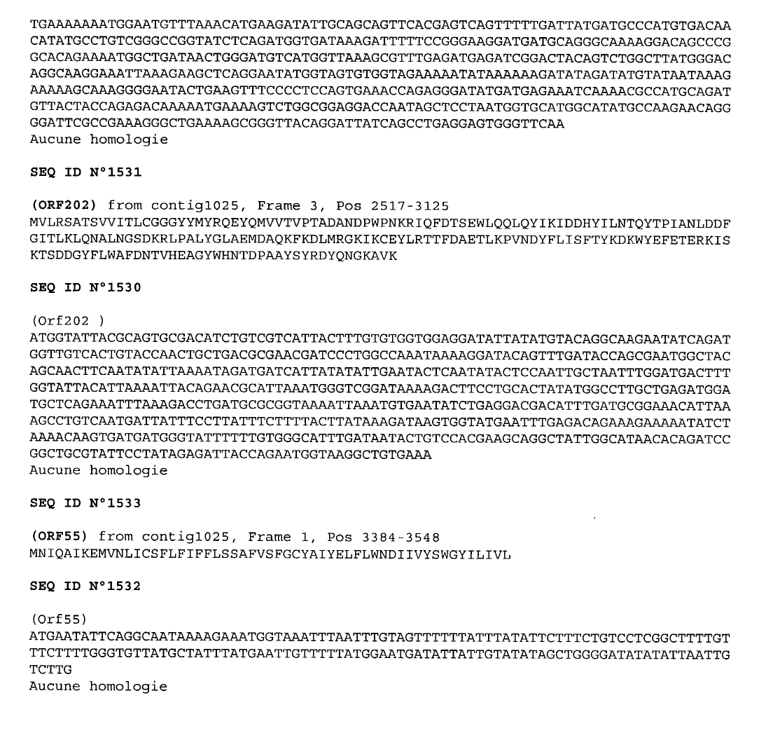

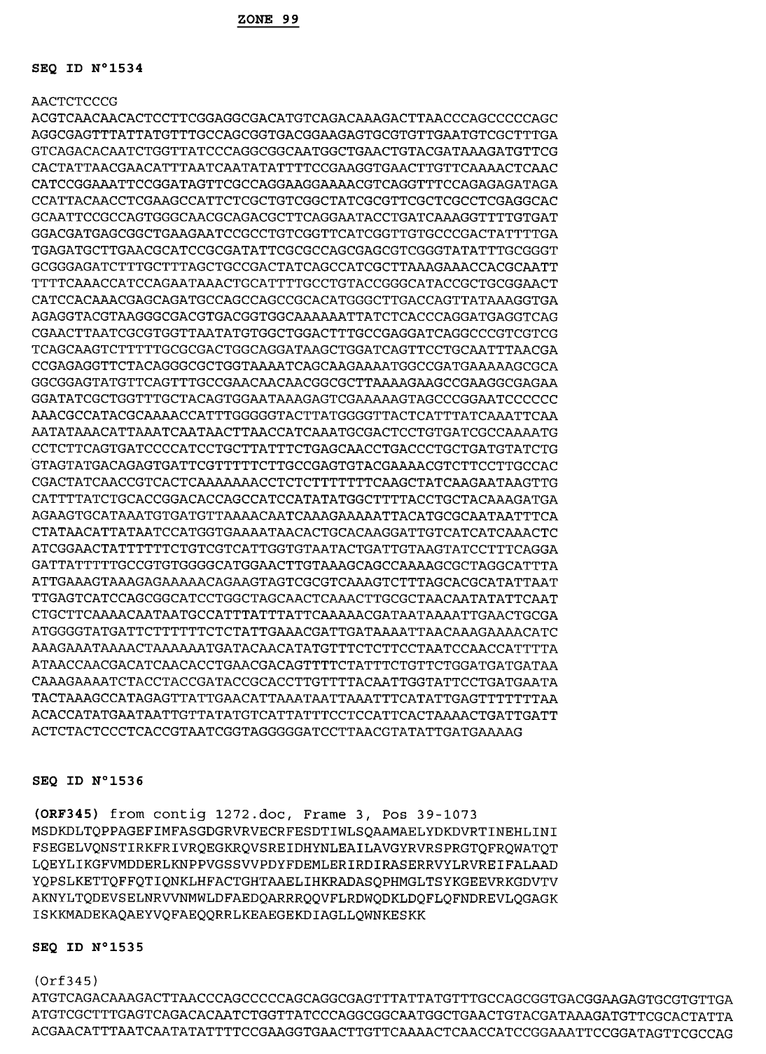

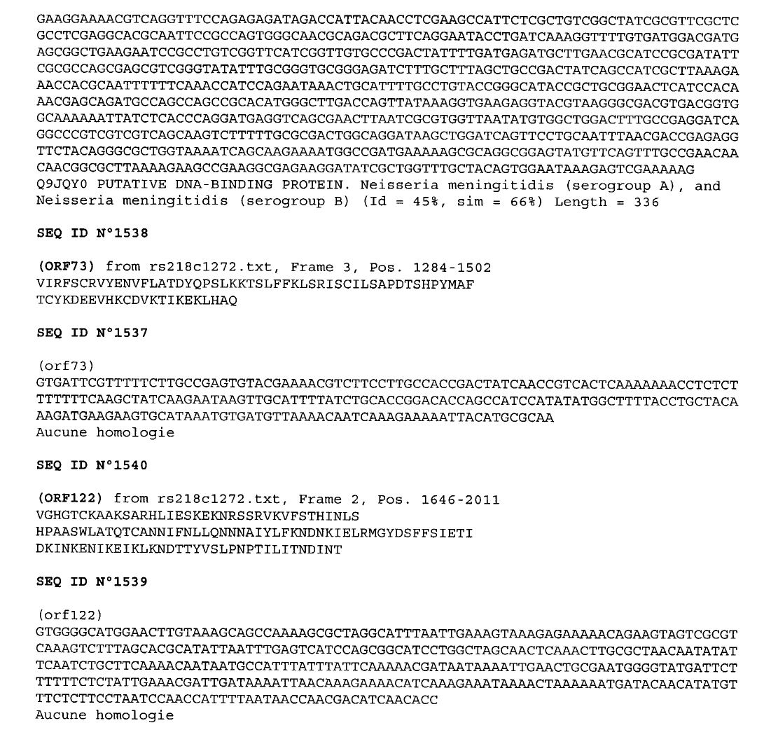

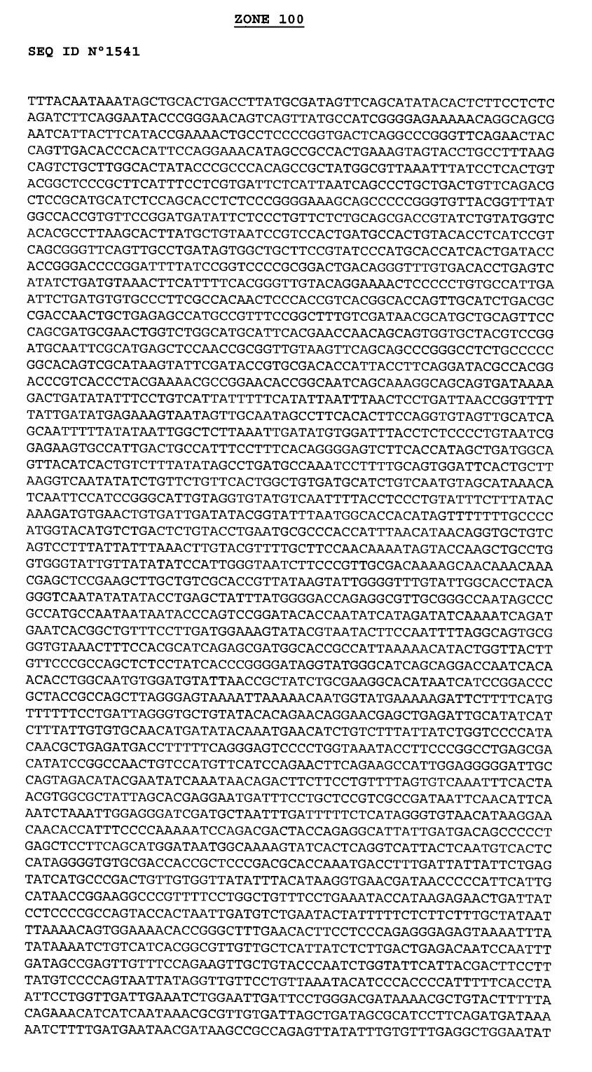

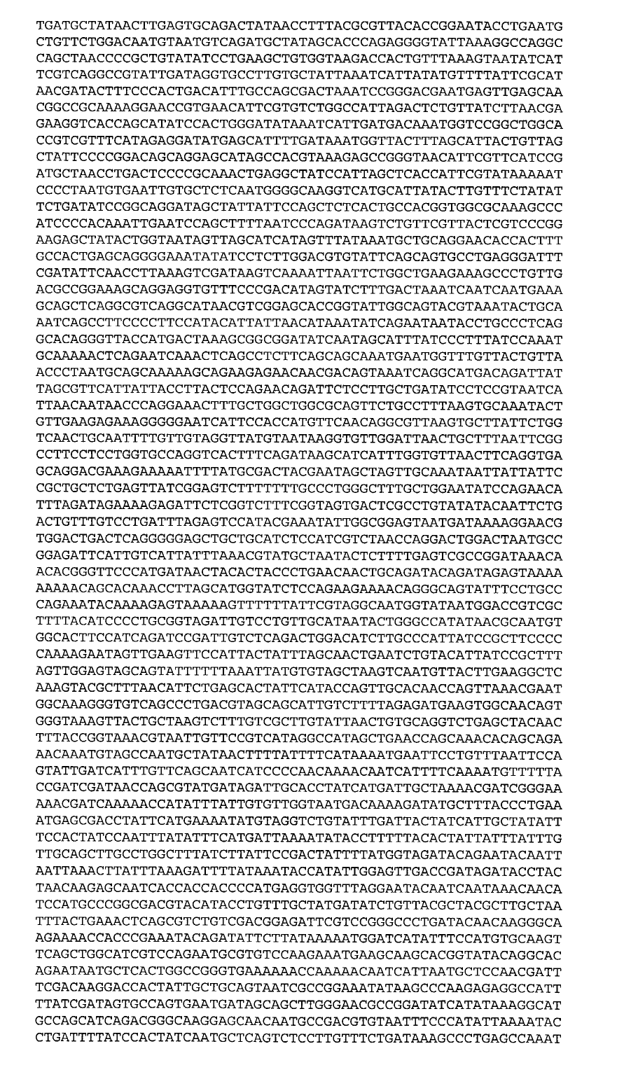

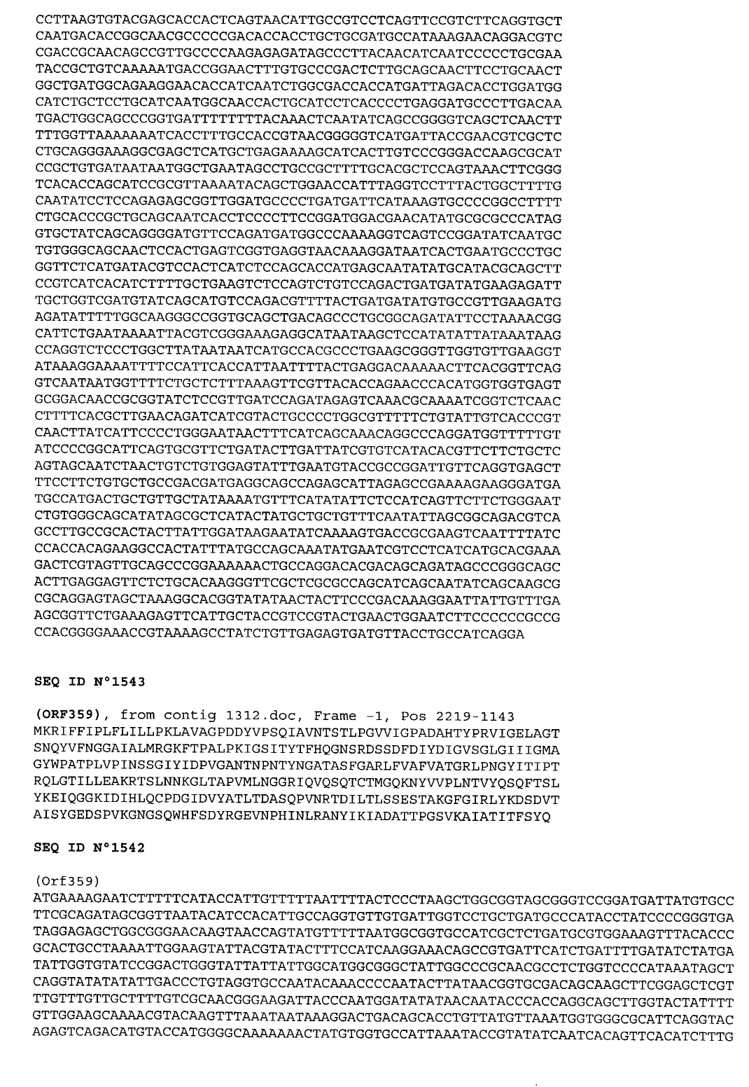

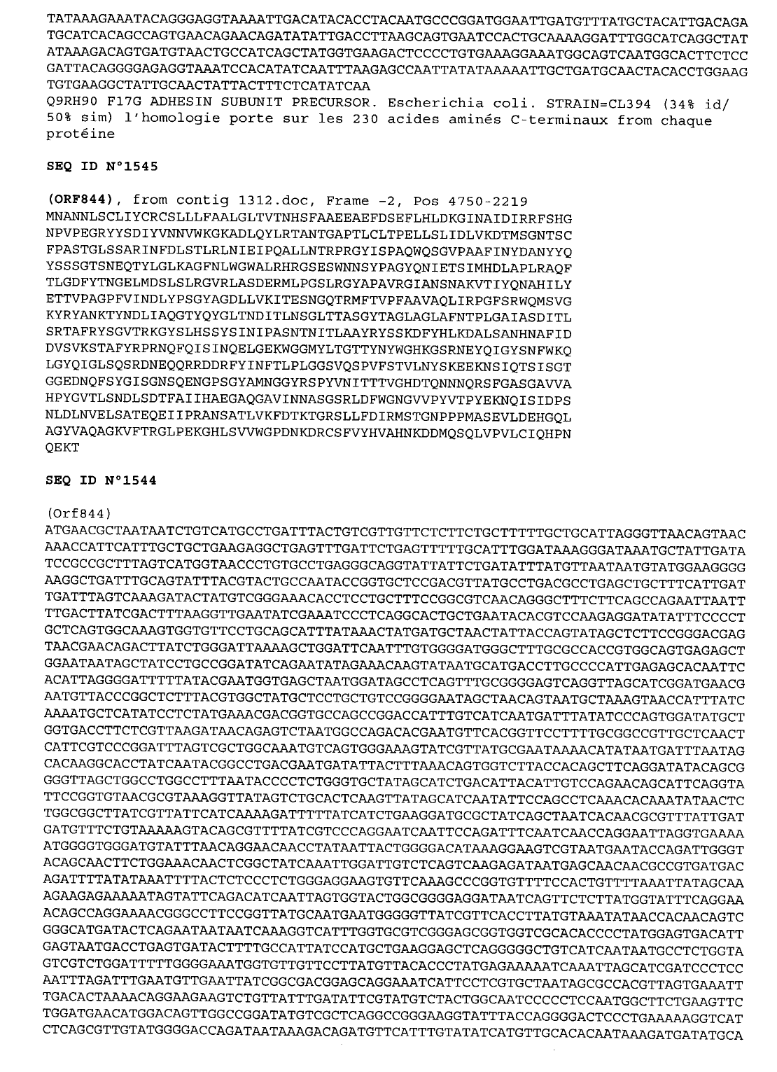

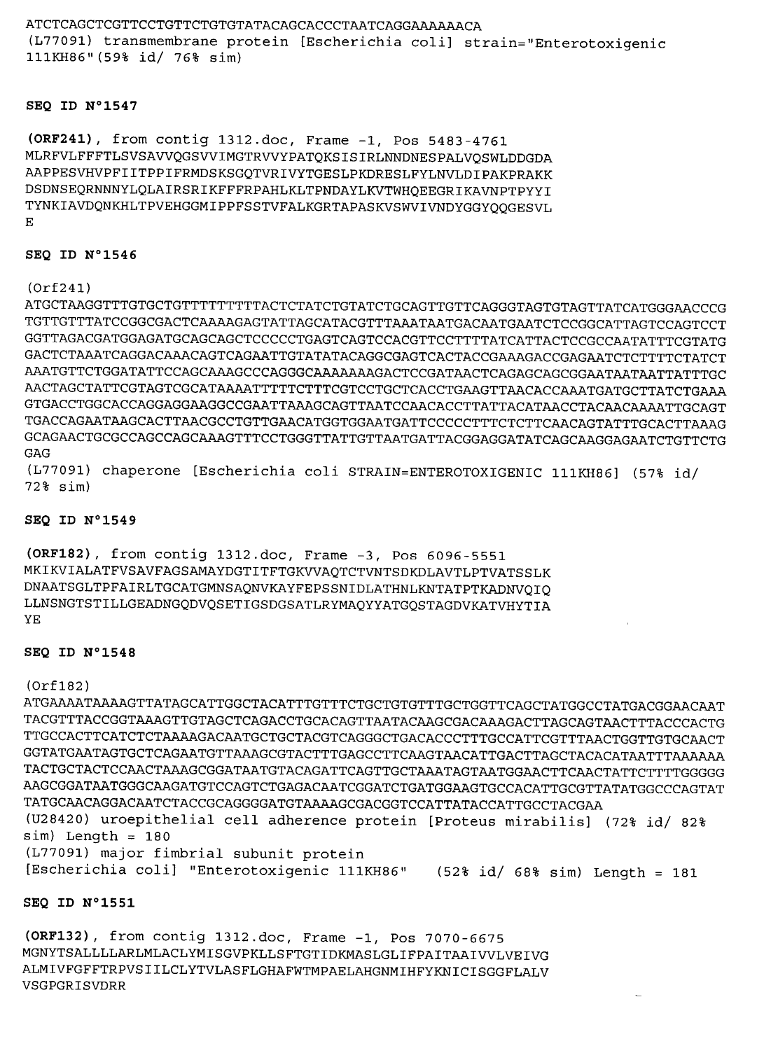

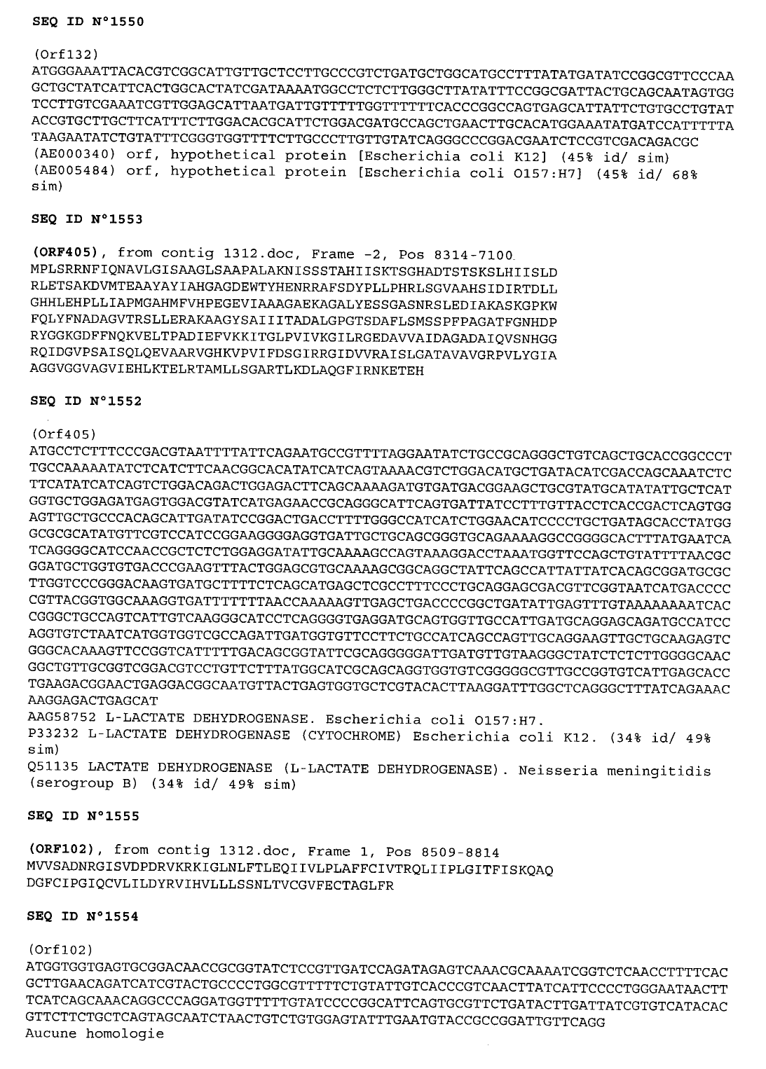

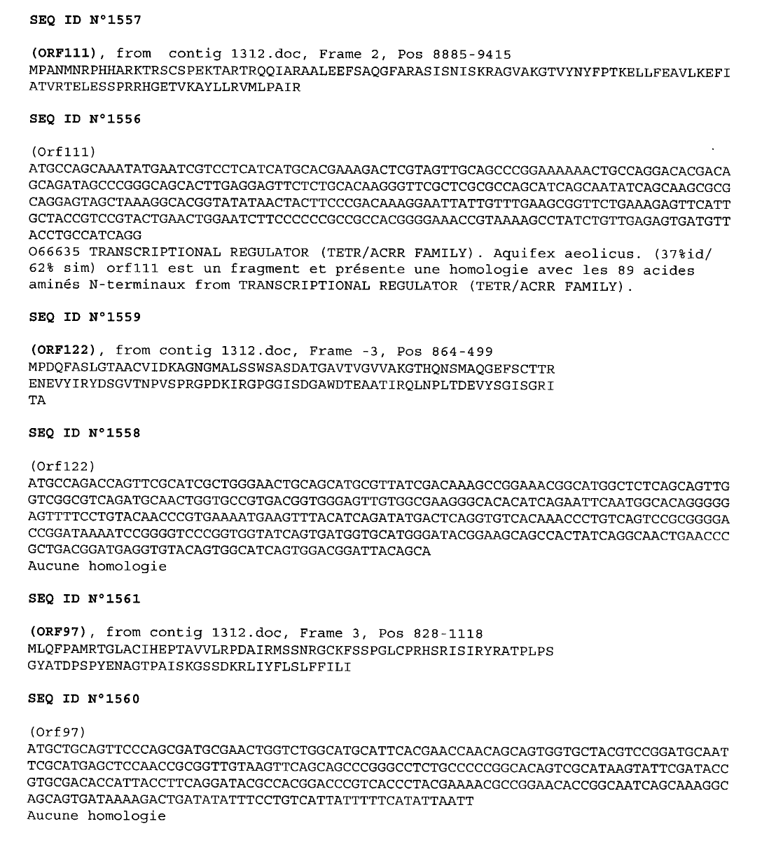

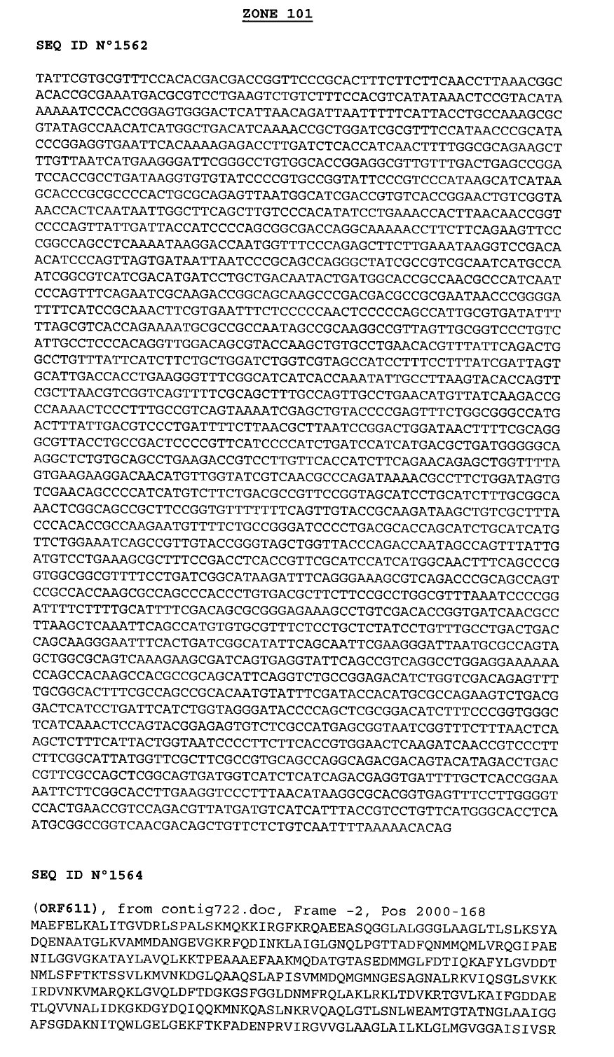

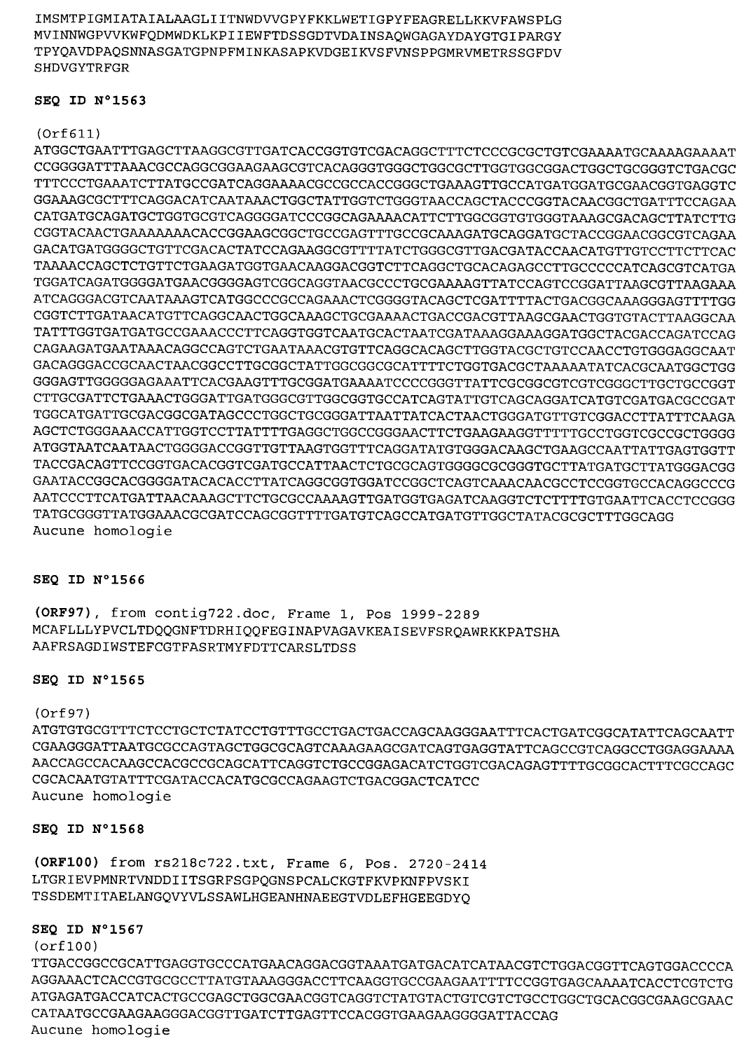

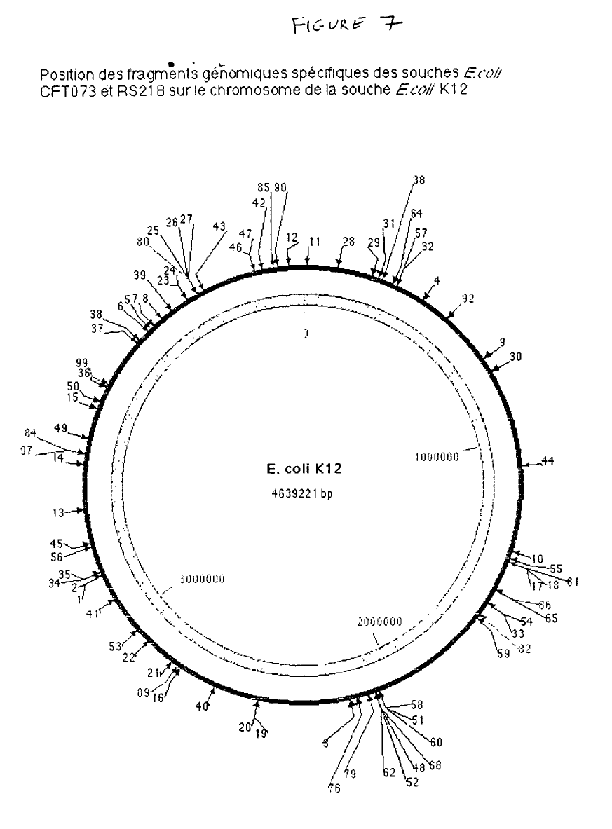

FIG. 7 represents the position of the genomic fragment specific to E. coli CFT073 and RS218 strains on the chromosome of E. coli K12 strain.

### EXAMPLE 1

Production of a C5+ A− Library and of a RS218+ K12− Library

Materials and Methods

Bacterial strains. The strains used for this subtractive hybridization are an E. coli isolated from the cerebrospinal fluid (CSF) of a newborn (E. coli CS of serotype 018:K1:H7; group B2) and two E. coli strains of group A, ECOR4 and ECOR15, which belong to the ECOR collection (ATCC). E. coli C5 harbours several virulent factors, such as the K1 capsular antigen, an sfa operon, the ibe 10 gene, Pap pili and the haemolysin (hly) gene. This strain belongs to the phylogenic group B2. In addition, the ECOR4 and ECOR15 strains, which belong to the phylogenic group A, express no identified virulence factor. Other E. coli strains were used: strain RS218 (serotype O18:K1:H7), an isolate from newborn CSF, which has been described in particular by Huang et al. 1995 (Infect. Immun. 63: 4470-4475) and which harbours the same virulence factors as the CS strain; and the E. coli K-12 laboratory strain MG1655 (group A), the genome of which has recently been sequenced (Blattner et al. 1997, Science 277: 1453-1461).

In addition, we used a set of 54 NMEC E. coli which could be associated with neonatal meningitis, obtained from the CSF of newborns suffering from meningitis (age range: 1 to 28 days) and belonging to the phylogenic group B2. This population was compared with the 15 E. coli strains of the phylogenic group B2 of the 72 strains from the ECOR collection, which are, themselves, not associated with meningitis. This collection is available from the ATCC (ATCC No. 3520 to No. 35391). These reference strains, isolated from various hosts and from various geographical sites are representative of the variation range in the E. coli species, and are divided into four main phylogenic groups (A, B1, B2 and D), four of them being unclassified. The bacteria were cultured at 37° C. in a Luria-Bertani broth or on Luria-Bertani agar. When necessary, ampicillin was used at a concentration of 100 μg per ml.

Southern transfer. The Southern transfers were carried out by capillary transfer onto positively charged nylon membranes. The hybridizations were carried out at 65° C. in 1% sodium dodecyl sulphate (SDS)—1M NaCl—50 mM Tris-HCl (pH 7.5)—1% blocking reagent (Boehringer Mannheim, Mannheim, Germany). The membranes were washed, first in 2×SSC (1×SSC corresponds to 0.15M NaCl+0.015M sodium citrate) for 15 min at room temperature, and then in 2×SSC—0.1% SDS for 30 min at 65° C. and finally, in 0.1×SSC for 5 min at room temperature. The detection by chemi-luminescence was carried out with the DIG luminescence detection kit for nucleic acid (Boehringer Mannheim) according to the manufacturer's instructions. The sfa and ibe 10 probes were produced by PCR using primers and an amplification method described previously (Bingen et al. 1997, J. Clin. Microbiol. 35: 2981-2982).

Representational difference analysis. The chromosomal DNA of the ECOR strains was randomly sheared by repeatedly pushing it through a hypodermic needle so as to obtain fragments with a length of between approximately 3 and 10 kb. This digested DNA was purified by phenol extraction. The chromosomal DNA of E. coli C5 was cleaved with the Sau3AI or Tsp509l restriction endonuclease. This DNA (20 μg) was ligated with 10 nmol of hybridized oligonucleotides RBam12 (5′-GATCCTCGGTGA-3′; SEQ ID NO: 154) and RBam24 (5′-AGGACTCTCCAGCCTCTCACCGAG-3′; SEQ ID NO: 155) or REco12 (5′-AATTCTCGGTGA-3′; SEQ ID NO: 156) and REco24 (5′-AGCACTCTCCAGCCTCTCACCGAG-3′; SEQ ID NO: 157) when the restriction was carried out with SauAI or Tsp5091, respectively. The DNA was separated from the excess primers by electrophoresis in a 2% low melting point agarose gel. The portion of the gel containing fragments longer than 200 bp was excized and digested with β-agarase. This DNA was purified by phenol extraction.

For the subtractive hybridization (first round), 0.2 μg of E. coli C5 DNA, ligated to the oligonucleotides, was mixed with 40 μg of fragmented ECOR4 or ECOR15 DNA in a total volume of 8 μl of 3×EE buffer (1×EE buffer corresponds to 10 mM N-(2-hydroxyethyl)piperazine-N′-(3-propanesulphonic acid); 1 mM EDTA [pH 8.0]). The solution was covered with mineral oil, and the DNA was denatured by heating to 100° C. for 2 min; 2 μl of 5M NaCl were added and the mixture was left to hybridize at 55° C. for 48 h. The reaction mixture was diluted 10 times in preheated 3×EE buffer—1M NaCl and immediately placed in ice. A portion of the dilution (10 μl) was added to 400 μl of PCR reaction mixture (10 mM Tris HCl [pH 9.0], 50 mM KCl, 1.5 mM MgCl₂, 0.1% Triton X-100, a 0.25 mM concentration of each deoxynucleotide triphosphate and 50 U of Taq polymerase per ml) and the whole mixture was incubated for 3 min at 70° C. in order to fill the ends of the re-hybridized E. coli C5 fragments. After denaturation at 94° C. for 5 min an addition of RBam24 or REco24 oligonucleotides (0.1 mmol per 100 μl), the hybridizations were amplified by PCR (30 cycles of 1 min at 94° C., 1 min at 70° C. and 3 min at 72° C., and then 1 min at 94° C. and 10 min at 72° C., in a GeneAmp 9600 thermocycler [Perkin-Elmer]). The PCR products were purified on a gel in order to separate the E. coli C5 fragments from the primer and from the high molecular weight ECOR subtraction DNA. The second round of subtractive hybridization was carried out using 40 μg of fragmented ECOR4 or ECOR15 E. coli DNA and 25 ng of DNA ligated to RBam24 or REco24 obtained from the first round. The products of the second round of subtraction were radiolabelled en masse and used as probe in Southern hybridization experiments, in order to verify that the amplified fragments were indeed unique to the DNA of the B2 strain and absent from the strains of group A. Thus, four subtractive libraries were produced.

Analysis of clones from the subtractive libraries. The DNA of the subtractive libraries was cloned into the BamH1 (Sau3AI libraries) or EcoR1 (Tsp5091 libraries) sites of pUC19 (New England Biolabs, Beverly, Mass.), and then transformed into Epicurian coli XL2-Blue ultra-competent cells (Stratagene, La Jolla, Calif.). The inserts were amplified by PCR reactions carried out on transformed colonies, using the following primers: P1 (5′-CATGCCTGCAGGTCGACTCT-3′; SEQ ID NO: 158) and P2 (5′-CGTTGTAAAACGACGGCCAG-3′; SEQ ID NO: 159). The clones were named according to the following (in order): the restriction enzyme used (“Tsp” or “Sau” ), the strain used for the subtraction (E4 or E15) and an alphanumeric name.

(i) DNA sequencing. After purification of the PCR products by reversible immobilization on a solid phase, the purified PCR fragments were sequenced using the Big-Dye Terminator Cycle Sequencing Ready Reaction kit with AmpliTaq DNA polymerase (Perkin-Elmer), on an ABI PRISM 377 XL automatic DNA sequencer (Perkin-Elmer), according to the manufacturer's instructions. When problems in obtaining a sequence of good quality were encountered with a given primer, the sequencing reaction was carried out with the dGDT Big-Dye Terminator Cycle Sequencing Ready Reaction kit (Perkin-Elmer). The sequences were screened for homologies with already published sequences, using the BLASTN and BLASTX computer programs (National Centre for Biotechnology Information, NCBI, Altschul et al. 1997, Nucleic Acids Res. 25: 3389-3402).

(ii) Southern-blot hybridization. In order to verify their specificity, the PCR products obtained, using the P1 and P2 primers, from the transformant colonies were labelled by incorporating digoxygenin-11-dUTP (Boehringer, Mannheim), and used as probes for the Southern-blot analysis of the DraI-digested chromosomal DNA originating from the E. coli C5, ECOR4 and ECOR15 strains and the E. coli K-12 strain MG1655.

(iii) Pulsed-field gel electrophoresis and mapping of the clones on the chromosomes of the RS218 and C5 strains. The position of the DNA sequences corresponding to the difference products cloned was determined with respect to the map of E. coli RS218 (Rode et al. 1999, Infect. Immun. 67:230-236) by probing Southern transfers of pulse-field agarose gels. The DNA of the strain RS218 was digested with BlnI, NotI and XbaI, and subjected to pulsed-field gel electrophoresis, as was the DNA of the strain C5, which was digested with BlnI and NotI. The gels were 1% agarose in 0.5×Tris-borate-EDTA buffer, and they were subjected to electrophoresis at 6 V/cm for 27 h, with pulsed durations varying in a linear manner between 2 and 49 s. The positions on the RS218 chromosome of sequences which are reactive with each of the clones were determined by comparing the BlnI and NotI recognized restriction fragments with the published macrorestriction map (Rode et al. 1999).

Results

Production of libraries of DNA fragments of the strain C5 of group B2, which are not found in the genome of E. coli of group A. Using the technique of representational difference analysis, we subtracted the chromosomes of two strains of group A (ECOR4 and ECOR15) from the chromosome of the strain of group B2 (strain C5). Four libraries were produced and named SauE4, SauE15, TspE4 and TspE15, according to the enzyme used to digest the chromosome of the strain C5 and the strain used for the subtraction. In each case, the amplified difference product from the second round of subtraction was labelled and used as probe against the DraI-digested DNA of C5, RS218, ECOR4 and ECOR15. Strong reactivity with the chromosome of the strains of group B2 was observed. In addition, there was little or no signal in the lanes corresponding to the subtractive strains (group A). 494 of the clones obtained were isolated for sequencing. Among them, 140 exhibit significant homology with the sequence of E. coli K-12, and were eliminated. Among the 354 remaining fragments, 259 sequences (SEQ ID NO: 1 to NO: 153, and NO: 170 to NO: 253) were unique. Table 1 below shows all the clones which exhibit significant homology with genes described previously.

TABLE 1
Summary of the BLAST search for the C5+ A− clones which exhibit significant
homologies^{a}

| Clone^{b} SEQ ID NO: | Size of | Database sequences | | | GenBank Accession |
|---|---|---|---|---|---|
| 170 to NO: 253 | insert (bp) | exhibiting similarity^{c} | Score | Probability | No. |
| SauE15.A7 | 190 | iroC (N), ATP cassette transporter | 157 | e⁻³⁷ | U62129 |
| | | (locus regulated by | | | |
| | | iron), Salmonella enterica | | | |
| | | serovar Typhi | | | |
| SauE15.B2 | 294 | repB (N), replication protein, | 123 | e⁻²⁶ | AF053946 |
| | | plasmid pCD1, Yersinia pestis | | | |
| SauE15.B6 | 157 | kps (N), promoter region of | 242 | e⁻⁶² | U05251 |
| | | region 3 of the polysialic acid | | | |
| | | gene cluster, E. coli | | | |
| SauE15.B9 | 107 | traD (N), sex factor F plasmid, | 198 | e⁻⁵⁰ | M29254 |
| | | E. coli | | | |
| SauE15.B10 | 240 | ORF 34 and 35 (P), 102 kb | 69 | e⁻¹² | CAA21357 |
| | | unstable region, Y. pestis | | | |
| SauE15.B12 | 479 | Unknown protein (P), E. coli | 102 | e⁻²¹ | AF044503 |
| | | ec11 | | | |
| SauE15.C1 | 100 | r6 (N), transposase, | 198 | e⁻⁴⁹ | AF081285 |
| | | pathogenicity island of E. coli | | | |
| | | CFT073 | | | |
| SauE15.C6 | 119 | IS100 (N), Yersinia pestis | 228 | e⁻⁵⁸ | L19030 |
| SauE15.C7 | 155 | TonB-dependent HI1217 receptor | 52 | e⁻⁷ | P45114 |
| | | precursor (P), Haemophilus | | | |
| | | influenzae | | | |
| SauE15.C9 | 273 | rhuM (N), pathogenicity island | 311 | e⁻⁸³ | AF106566 |
| | | of Salmonella enterica serovar | | | |
| | | Typhimurium (SPI 3) | | | |
| SauE15.C11 | 77 | orfE (N), distal region of the | 129 | e⁻²⁹ | X55815 |
| | | tra operon promoter, plasmid | | | |
| | | R100, S. flexneri | | | |
| SauE15.D4 | 153 | IS100 (n), Y. pestis | 287 | e⁻⁷⁶ | L19030 |
| SauE15.D8 | 347 | r3 (N) , beta-cystathionase, | 615 | e⁻¹⁷⁴ | AF081286 |
| | | pathogenicity island of E. coli | | | |
| | | CFT073 | | | |
| SauE15.E4 | 281 | senB (N), enterotoxin E. coli | 541 | e⁻¹⁵² | Z54195 |
| SauE15.E11 | 314 | traJ, Y (N), plasmid R1-19, | 523 | e⁻¹⁴⁷ | M19710 |
| | | E. coli | | | |
| SauE15.F3 | 422 | chuA (P), gene for haem use, | 98 | e⁻²⁰ | U67920 |
| | | E. coli 0157: H7 | | | |
| SauE15.F9 | 137 | Thacesterase (P), Bacillus sp | 48 | e⁻⁶ | AB016427 |
| SauE15.F10 | 210 | r3 (N), beta-cystathionase, | 408 | e⁻¹¹² | AF081286 |
| | | pathogenicity island of E. coli | | | |
| | | CFT073 | | | |
| SauE15.G3 | 206 | traG (N), plasmid R100, S. | 165 | e⁻³⁹ | U01159 |
| | | flexneri | | | |
| SauE15.G6 | 328 | IS100 (N), Y. pestis | 480 | e⁻¹³⁴ | L19030 |
| SauE15.H5 | 200 | HMWP1 protein (P), Yersinia | 80 | e⁻¹⁵ | CAA73127 |
| | | enterocolitica | | | |
| SauE15.H7 | 150 | Oxidoreductase (P), Thermotoga | 160 | e⁻¹¹ | AE001762 |
| | | maritima | | | |
| SauE15.H10 | 141 | traT (N), plasmid R100, E. coli | 280 | e⁻⁷⁴ | J01769 |
| SauE15.H11 | 160 | Haemoglobin protease (p), | 50 | e⁻⁶ | CAA11507 |
| | | E. coli EB1 | | | |
| SauE15.I3 | 176 | asst (N), aryl sulphate sulpho- | 341 | e⁻⁹² | U32616 |
| | | transferase, Klebsiella sp. | | | |
| SauE15.I11 | 162 | chuA (N), gene for haem use, | 305 | e⁻⁸² | U67920 |
| | | E. coli 0157: H7 | | | |
| SauE15.J7 | 118 | troB (N), glucosyl transferase | 74 | e⁻¹² | U62129 |
| | | homologue S. typhi | | | |
| SauE15.J9 | 96 | IS100 (N), Y. pestis | 174 | e⁻⁴² | L19030 |
| SauE15.M4 | 193 | r3 and malX (N), pathogenicity | 383 | e⁻¹⁰⁴ | AF081286 |
| | | island of E. coli CFT073 | | | |
| SauE15.M8 | 149 | De1ta- (L- α-aminoadipyl) -L- | 65 | e⁻¹¹ | P26046 |
| | | cysteinyl-D-valine synthetase | | | |
| | | (P) Penicillium sp. | | | |
| SauE15.M12 | 119 | senB (N), enterotoxin of | 228 | e⁻⁵⁸ | Z54195 |
| | | enteroinvasive E. coli | | | |
| SauE15.N7 | 188 | Plasmid pColBM-C1139 (N), | 208 | e⁻⁵² | M35683 |
| | | E. coli | | | |
| SauE4.A2 | 321 | orf 36 (N), 102 kb unstable | 135 | e⁻³⁰ | AL031866 |
| | | region of Y. pestis | | | |
| SauE4.A5 | 249 | r3 (N), beta-cystathionase, | 355 | e⁻⁹⁶ | AF081286 |
| | | pathogenicity island of E. coli | | | |
| | | CFT073 | | | |
| SauE4.B4 | 360 | IS200 (N), E. coli | 523 | e⁻¹⁴⁷ | L25845 |
| SauE4.C7 | 275 | Hippurate hydrolase (P), | 54 | e⁻⁷ | P45493 |
| | | Campylobacter jejuni | | | |
| SauE4.C11 | 255 | Pristinamycin I synthetase (p), | 51 | e⁻⁶ | CAA67248 |
| | | Streptomyces spp. | | | |
| SauE4.D3 | 239 | hlyB, (N), haemolysin, E. coli | 474 | e⁻¹³² | M81823 |
| SauE4.E3 | 263 | shuX genes (N), genes for haem | 387 | e⁻¹⁰⁶ | U64516 |
| | | use Shigella dysenteriae | | | |
| SauE4.E11 | 242 | IS66 (N), E. coli | 329 | e⁻⁸⁸ | AF119170 |
| SauE4.F8 | 188 | sorC genes (N), sor operon for | 139 | e⁻³¹ | X66059 |
| | | L-sorbose use, Klebsiella | | | |
| | | pneumoniae | | | |
| SauE4.F9 | 439 | YfkN (P) Bacillus subtilis | 57 | e⁻⁸ | BAA23404 |
| SauE4.F12 | 324 | kpsM (N), region 3 of the | 642 | 0 | M57382 |
| | | polysialic acid gene cluster, | | | |
| | | E. coli K1 | | | |
| SauE4.H2 | 85 | sorM (N), sor operon for | 105 | e⁻²² | X66059 |
| | | L-sorbose use, K. pneumoniae | | | |
| SauE4.I2 | 431 | yihA, (N), plasmid R100, | 829 | 0 | AP000342 |
| | | S. flexneri | | | |
| TspE4.A5 | 271 | pap and prsK (N), pili | 498 | e⁻¹³⁹ | X61239 |
| | | P-protein, E. coli | | | |
| TspE4.A8 | 216 | 17 kD orf of the pili prs | 387 | e⁻¹⁰⁶ | X61238 |
| | | operon (N), cytoplasmic | | | |
| | | protein, E. coli | | | |
| TspE4.A9 | 179 | kpsT (N), region 3 of the | 347 | e⁻⁹⁴ | M57381 |
| | | polysialic acid gene cluster, | | | |
| | | E. coli K1 | | | |
| TspE4.A10 | 212 | HecB (P), putative transporter | 73 | e⁻¹³ | AAC31980 |
| | | of the haemolysin activator, | | | |
| | | Erwinia chrysanthemi | | | |
| TspE4.B1 | 229 | r1 (N), pathogenicity island of | 430 | e⁻¹¹⁹ | AF081286 |
| | | E. coli CFT073 | | | |
| TspE4.B5 | 215 | Sensory transduction histidine | 52 | e⁻⁷ | BAA18223 |
| | | kinase (P), Synechocystis sp. | | | |
| TspE4.B9 | 319 | senB (N), enterotoxin of | 617 | e⁻¹⁷⁵ | Z54195 |
| | | enteroinvasive E. coli | | | |
| TspE4.B12 | 430 | IS100 (N), Y. pestis | 698 | 0 | L19030 |
| TspE4.C10 | 267 | Intergenic capsular cluster (N) | 466 | e⁻¹²⁹ | AF118251 |
| | | of E. coli K42 | | | |
| TspE4.D2 | 232 | waaL (N), lipid A core of | 404 | e⁻¹¹¹ | AF019746 |
| | | E. coli : surface polymer ligase | | | |
| TspE4.D4 | 245 | orf 169 (N), plasmid F, E. coli | 456 | e⁻¹²⁶ | X17539 |
| TspE4.D10 | 222 | cnfl (N) cytotoxic necrosis | 440 | e⁻¹²² | X70670 |
| | | factor, E. coli | | | |
| TspE4.D11 | 217 | hlyB (N), haemolysin, E. coli | 422 | e⁻¹¹⁷ | M81823 |
| TspE4.E3 | 298 | hlyD (N), haemolysin, E. coli | 553 | e⁻¹⁵⁶ | M10133 |
| TspE4.E4 | 267 | orf 95 (N), plasmid F, E. coli | 482 | e⁻¹³⁴ | X17539 |
| TspE4.E6 | 190 | L-sorbose P reductase (P), | 112 | e⁻²⁵ | P37084 |
| | | K. pneumoniae | | | |
| TspE4.E8 | 285 | hlyB (N) haemolysin, E. coli | 541 | e⁻¹⁵² | M81823 |
| TspE4.G7 | 238 | tra (N), plasmid F, E. coli | 448 | e⁻¹²⁴ | X61575 |
| TspE4.G8 | 323 | Transmembrane protein (P), | 82 | e⁻¹⁵ | AAA92620 |
| | | E. coli | | | |
| TspE4.H1 | 283 | Arginine deiminase (P) | 63 | e⁻¹⁰ | P13981 |
| | | Pseudomonas aeruginosa | | | |
| TspE4.H9 | 179 | traT (N), plasmid R100, E. coli | 353 | e⁻⁹⁶ | J01769 |
| TspE4.H10 | 223 | prf and papI (N), adhesin | 418 | e⁻¹¹⁵ | X76613 |
| | | regulatory gene, E. coli | | | |
| TspE4.H11 | 279 | orf 9 (N) , plasmid F, E. coli | 456 | e⁻¹²⁷ | X17539 |
| TspE4.I10 | 269 | neuC (N), capsule gene cluster, | 492 | e⁻¹³⁷ | M84026 |
| | | E. coli | | | |
| TspE4.J1 | 327 | yhtA (N), plasmid R100, E. coli | 521 | e⁻¹⁴⁶ | AP000342 |
| TspE4.J6 | 221 | chuA (N), gene for haem use, | 375 | e⁻¹⁰² | U67920 |
| | | E. coli O157:H7 | | | |
| TspE4.K3 | 180 | iss (N), survival in serum, | 270 | e⁻⁷⁰ | AF042279 |
| | | E. coli | | | |
| TspE4.K8 | 184 | IS100 (N), E. coli prf and papB | 190 | e⁻⁴⁷ | L19030 |
| | | (N), E. coli | 143 | e⁻³² | X76613 |
| TspE15.A1 | 332 | Na⁺/H⁺antiporter (P), | 96 | e⁻²⁰ | Q57007 |
| | | H. influenzae | | | |
| TspE15.C1 | 299 | hra (N), heat-resistant | 537 | e⁻¹⁵¹ | U07174 |
| | | agglutinin, E. coli 99 | | | |
| TspE15.C3 | 386 | hcp (N), E. coli | 81 | e⁻¹⁴ | AF044503 |
| TspE15.D7 | 239 | STBA protein (P), plasmid NR1, | 87 | e⁻¹⁷ | P11904 |
| | | E. coli | | | |
| TspE15.D9 | 230 | chuA (P), gene for haem use, | 89 | e⁻¹⁸ | AAC44857 |
| | | E. coli O157:H7 | | | |
| TspE15.E7 | 360 | kpsS (N), region 1 of the | 531 | e⁻¹⁴⁹ | X74567 |
| | | capsule gene cluster, E. coli | | | |
| | | K5 | | | |
| TspE15.G12 | 287 | Putative aminotransferase (P), | 72 | e⁻¹² | Q08432 |
| | | B. subtilis | | | |
| TspE15.H2 | 258 | Pyruvate formate lyase | 51 | e⁻⁶ | AAB89799 |
| | | activation enzyme (P), | | | |
| | | Streptococcus mutans | | | |
| TspE15.H5 | 310 | cnfl (N) cytotoxic necrosis | 601 | e⁻¹⁷⁰ | U42629 |
| | | factor, E. coli | | | |
| TspE15.H9 | 273 | Major fimbrial subunit of | 48 | e⁻⁵ | I41206 |
| | | fimbriae resembling F17 (P), | | | |
| | | E. coli | | | |
| TspE15.I2 | 112 | prs and papE (N), pili-P | 222 | e⁻⁵⁷ | X62158 |
| | | protein, E. coli | | | |

^{a}Only the homologies with a probability of at least e⁻⁵ were selected. The homologies with bacterio-phages (n = 21) are not given.
^{b}The clones are named according to the name of the enzyme followed by the name of the strain used for the subtraction (E4 or E15) and a code composed of one letter and one number.
^{c}The name of the sequence of the gene is given (with the type of similarity between brackets), followed by the product or by the function which is encoded by the gene and/or the location of the gene, and also the name of the organism. N, similarity at the nucleotide level; P, similarity at the protein level; ORF, open reading frame.

Some of the clones correspond to genes which are already known to be present in the strain C5, such as pap, hly and kps. Among the 494 clones, none proved to be homologous to sfa or ibe10. Additional rounds of subtraction and/or the sequencing of additional clones make it possible to obtain greater exhaustiveness, until this is complete. In addition, sequences are found which correspond to virulence factors described in strains which, to date, have never been associated with neonatal meningitis: (i) prs, cnf and hra, all of which form part of a PAI (pathogenicity island) in the uropathogenic E. coli strain J96; (ii) chuA, a gene involved in the iron transport system and found in enterohaemorrhagic E. coli 0157:H7; and (iii) senB, a gene encoding an enterotoxin on the virulence plasmid of enteroinvasive E. coli and Shigella. Finally, 153 of the fragments sequenced (SEQ ID NO: 1 to NO: 153) exhibited no significant homology with any published sequence.

Mapping of the sequences specific for NMEC, on the chromosome of E. coli. The availability of a physical map of the chromosome of E. coli RS218 (Rode et al. 1999, Infect. Immun. 67: 230-236) made possible the investigation of the distribution of the sequences mentioned above. Of the 64 clones which were chosen for this purpose, 7 exhibit homology with known virulence factors (kps, hly, prs, hra, cnf1, chuA and senB) and 57 exhibit no known homology. These latter clones were chosen randomly from the TspE4 and SauE15 libraries. These two libraries were chosen because they contain most of the genes expected in the B2s (for example, pap, hly and kps), and were therefore considered to be sufficiently complete to be representative. All the clones exhibited homology, by Southern hybridization, with respect to the chromosome of the strain RS218. The PCR products of these clones were labelled and used to probe Southern transfers of RS218 DNA digested with the BlnI, NotI and XbaI enzymes (enzymes which cleave infrequently). The location of both sfa and ibe10 was determined. In order to evaluate the B2+ A− specificity thereof, each of these clones was used to probe the DraI-digested DNA of strains ECOR4, ECOR15 and MG1655: they proved to be nonreactive with respect to these strains.

The mapping of these clones revealed a non-random distribution of the C5+ A− sequences. This distribution is illustrated by FIG. 1. In this figure, the upper arrows indicate the six regions which were found in this study and which are represented by NotI and BlnI fragments with a high density of clones specific for C5. The clones exhibiting known homologies are indicated, as are the positions of the sfa and ibe10 probes. Region 6 was divided into two subregions according to the mapping of the clones on various XbaI fragments. The exponents next to the names of the clones indicate the following: 1, TspE4.A7 was positioned by SauE15.F12 overlap; 2, SauE15.F12 also exhibits reactivity on the plasmid; and 3, TspE4.A8 also exhibits weak reactivity on NotI fragment P.

Forty-four of the clones were clustered in six distinct groups on the chromosome. One clone encoding a portion of the chuA gene found in enterohaemorrhagic E. coli was not associated with any of these clusters and remained isolated. Region 1 is contained in BlnI fragment m (85 kb). The clones of region 2 were mapped on NotI fragments p and n (˜240 kb). Region 3 is contained in NotI fragment a (˜310 kb) and region 4 is contained in BlnI fragment h (210 kb). Region 5 is located on BlnI fragment j (135 kb). Region 6, which overlaps NotI fragment b and BlnI fragment b (˜550 kb), was divided into two subregions, regions 6a and 6b, with the XbaI enzyme. This latter subregion contains clones which exhibit homologies with Cnfl, hly, prs and hra. The sfa and ibe10 probes hybridize in regions 2 and 6a, respectively. The genes encoding the capsule were not linked to any of these regions. Six clones exhibiting no homology with known sequences and the senB gene were all located on a large plasmid present in the strain RS218.

Distribution of the C5+ A− genomic regions among two collections of E. coli strains. In order to refine the relevance of these regions in terms of diagnosis and pathogenesis, we determined the frequency of the appearance of clones located on regions 1, 3, 4, 5 and 6b, and also the clone containing a portion of the chuA gene, in a collection of 54 E. coli which were associated with neonatal meningitis and which belong to the phylogenic group B2 (54 NMEC). We excluded regions 2 and 6a from this study since they contain the sfa and ibe10 genes, as it has been possible to establish the distribution thereof among the E. coli strains of group B2. For each region, two to four clones were used independently to screen Southern transfers of the genomic DNA of NMEC isolates. The control group corresponded to the 15 B2 strains of the ECOR collection; the strains have not, to date, been associated with meningitis. The results are given in Table 2 below.

TABLE 2
Strains isolated from cases of neonatal meningitis and from the ECOR collection,
which hybridized with the subtractive clones used as probe
% of strains positive for hybridization by Southern transfer,
with given clones representive of various regions or homologous to chuA

| | | | | | | | | | | | (Region 6b) | | |
|---|---|---|---|---|---|---|---|---|---|---|---|---|---|
| | | (Region 1) | | (Region 3) | | (Region 4) | | (Region 5) | | | chuA | | |
| | | TspE4. | TspE4. | SauE15. | TspE4. | SauE15. | SauE15. | TspE4. | SauE15. | TspE4. | SauE15. | PAI | TspE4. |
| Source | n | K6 | H5 | M9 | H6 | L4 | K12 | C4 | M10 | C2 | N4 | V^{a} | J6 |
| Meningitis | 54 | 91^{b} | 91^{b} | 80^{b} | 80^{b} | 81^{b} | 81^{b} | 81^{b} | 100 | 98^{c} | 17^{b} | 17^{b} | 100 |
| belonging to the | | | | | | | | | | | | | |
| phylogenic group | | | | | | | | | | | | | |
| B2 | | | | | | | | | | | | | |
| ECOR collection, | 15 | 40 | 40 | 13 | 13 | 47 | 47 | 47 | 100 | 87 | 47 | 47 | 100 |
| phylogenic group | | | | | | | | | | | | | |
| B2 | | | | | | | | | | | | | |

^{a}The prevalence of PAI V was evaluated using clones TspE4.D11, TspE4.D10 and TspE15.C1, which are homologous to the hly, cnf1 and hra genes, respectively.
^{b}p < 0.05, with respect to the strains of the ECOR collection (the existence of a difference in the distribution of the clones studied was tested using the χ² test).
^{c}Nonsignificant, with respect to the strains of the ECOR collection.

All the regions mentioned above are widely present among the NMEC, except region 6b which is under-represented in the strains associated with meningitis. In addition, regions 1, 3 and 4 appeared with a frequency which was notably higher (p<0.05) among the NMEC than among the other B2 strains, thus suggesting that these regions contain DNAs encoding NMEC-specific factors. These regions, their portions and the clones which they contain therefore constitute a source of polynucleotides involved in neonatal meningitis. They can, therefore, be used as active principles in anti-neonatal meningitis vaccines, and allow the development of medicinal products intended to prevent, alleviate or treat neonatal meningitis using products which slow down or block the transcription and/or translation of these polynucleotides, or which slow down or block the activity of the polypeptides which they encode.

Discussion

In these studies, we carried out a subtractive hybridization in order to identify the regions of the chromosome which are capable of encoding phylogenic attributes of interest. We carried out two rounds of subtractive hybridization, choosing to subtract the DNA population of a B2 E. coli (E. coli C5 associated with neonatal meningitis) from that of E. coli strains of group A. We thus obtained libraries of C5+ A− clones containing inserts ranging from 100 to 500 bp long in which the specific NMEC clones could also be identified. Among the 259 clones representative of these libraries, 153 are novel as products (SEQ ID NO: 1 to NO: 153), the other fragments exhibit homology with known products. Among the fragments with homology, some are described for the first time as being of nature C5+ A−; this is in particular the case of chuA (ATCC Accession Number U67920) and of the four clones which appear to correspond to it: TspE4J6 (SEQ ID NO: 241, 221 bp, probability of e⁻¹⁰², score of 375), SauE15.I11 (SEQ ID NO: 195, 162 bp, probability of e⁻⁸², score of 305), SauE15.F3 (SEQ ID NO: 185, 422 bp, probability of e⁻²⁰, score of 98) and TspE15.D9 (SEQ ID NO: 248, 230 bp, probability of e⁻¹⁸, score of 89). It is also the case of the island PAIV, and of regions 1, 6a and 6b which were identified (cf. examples). The invention also demonstrates that these DNA fragments are not dispersed randomly on the chromosome of E. coli, and that there are chromosomal regions of E. coli which are of nature C5+ A− (regions 1, 2, 3, 4, 5, 6a and 6b).

The specificity of the subtractive libraries was evaluated (i) by Southern transfer with nonpathogenic strains, and (ii) by sequence analyses which showed 72% of the clones exhibit no homology with the published sequence of E. coli K-12. Some clones correspond to genes associated with virulence (kps, pap and hly). On the other hand, we have not isolated any clone corresponding to the sfa and ibe10 genes. However, clones derived from regions containing these two genes were obtained. Taken together, these data confirm the complete nature of these libraries.

In addition, while, in the examples described above, 494 clones were isolated for sequencing and made it possible to obtain 259 clones which are different from each other and which exhibit no significant homology with E. coli K12, it will become clearly apparent to those skilled in the art that the initial number of clones initially isolated and sequenced can, if desired, be considerably increased, for example by starting with 3000 clones or more instead of 494. Automatic sequencing machines make it possible to easily treat such a number of clones. Increasing the number of clones initially sequenced makes it possible, in particular, to increase the final set of clones which are different from each other and which exhibit no significant homology with E. coli K12. This makes it possible to increase the level of exhaustiveness of the set obtained. Alternatively, or in combination, it is possible to choose to increase the number of strains from which the DNA population originates (B2 E. coli) and/or to increase the number of subtractive libraries prepared (using other restriction enzymes). In order to increase the level of specificity obtained, it is possible to choose to multiply the subtraction cycles: in the example described above, only two cycles were necessary to obtain said 259 clones, but it is possible to choose to carry out a third or a fourth cycle. It is also possible to choose to use other restriction enzymes, any enzyme allowing fragments of approximately 100 to 1 500 bp, with a mean of approximately 300 to 500 bp, to be obtained being, a priori, the most suitable (i.e. any restriction enzyme having short restriction sites, for example 4 bp, such as Tsp509I or Sau3AI, and also MspI or MaeII). Advantageously, two to three rounds of subtraction followed by the elimination of those of the clones which exhibit homology with a strain of group A such as E. coli K12 gives a very good level of specificity. Varying the nature of the restriction enzymes used, in particular between each subtraction cycle, further increases the level of exhaustiveness and/or of specificity of the set obtained. In order to evaluate the level of exhaustiveness effectively obtained, this evaluation can be carried out by investigating the presence or absence of known markers such as sfa or ibe10.

In the C5+ A− set isolated herein, particularly discriminating DNAs can be identified (cf. Table 2 above): most of them (more than 80% of them) are in fact present in the E. coli of group B2 of the ECOR collection at a frequency greater than 40% (clones of regions 1, 4, 5 and 6b), some of them even being present at a frequency greater than 50%, or even greater than 80% (clones of region 5 and clones corresponding to chuA), reaching a B2 frequency of 100% for approximately 15% of the set obtained (some clones of region 5 and isolated clone corresponding to chuA).

Seven C5+ A− regions (regions 1, 2, 3, 4, 5, 6a and 6b) were also identified. An epidemiological approach was undertaken in order to study the role of these regions in the infectious process of NMEC (E. coli which it has been possible to associate with neonatal meningitis). Given that the majority of NMEC belong to the phylogenic group B2, we determined the prevalence of each region, and also of chuA, among NMEC of group B2, and among the strains of group B2 not associated with meningitis (ECOR collection). Although small, this control group was chosen since it is composed of reference strains from the ECOR collection, which is considered to be representative of the range of genotypic variations of the species. We used two to four clones of each region and the TspE4.J6 clone (SEQ ID NO: 241), a homologue of chuA, as probes against Southern transfers of genomic DNA prepared from isolates belonging to the two groups. The presence of this clone among the meningitis isolates indicates that all these regions, except region 6b, are widely represented among NMEC, thus suggesting the involvement of genes encoded by these regions in the pathogenesis of these strains. On the other hand, and surprisingly, region 6b, which resembles PAI V, has a low prevalence in NMEC (17%) but is widely represented in the strains of group B2 of the ECOR collection (47%). Region 5 has a high prevalence, but it is similar in both collections and may thus correspond to segments which are highly characteristic of the phylogenic group B2 with respect to the group A. Region 5 of E. coli thus appears to be an advantageous source of DNA fragments present in a great majority (more than 80%) of E. coli of group B2 of the ECOR collection, and absent from the majority of E. coli of group A of the ECOR collection. The same distribution was observed for chuA, but interestingly, these genes were present, without exception, in all the strains of group B2 tested. As regards regions 1, 3 and 4, they appear to be clearly more common in NMEC than in the other B2 E. coli . Given that regions 1, 3 and 4 do not, moreover, contain any known virulence factors, these regions appear to correspond to DNA islands associated with invasion of the meninges by E. coli in infants and newborn.

It may be noted that, in the prior art, the sequences of regions 1, 3, 4 and 5 were not available, that they had never been described or isolated and that no function was known for them. Regions 1, 3, 4 and 5 therefore appear to be novel. As regards regions 2, 6a and 6b, they comprise DNAs which were known as products, but this is the first description of the existence of such regions and the first description of their nature C5+ A−.

### EXAMPLE 2

Distribution of the C5+ A− Fragments Among the ECOR Strains

The frequency of presence of the fragments obtained in Example 1 (C5+ A− fragments) among ECOR E. coli of groups B1 and D was then measured by Southern hybridization as described in Example 1. The results obtained for 14 of them (SEQ ID NO: 56, 116, 43, 51, 141, 130, 45, 50, 52, 119, 127, 125, 55 and 37) are given in Table 3 below.

TABLE 3

| ECOR | PREVALENCE OF THE SUBTRACTIVE CLONES WITH NO HOMOLOGY, IN THE STRAINS | | | | | | | |
|---|---|---|---|---|---|---|---|---|
| COLLECTION | OF THE ECOR COLLECTION | | | | | | | |
| GROUPS | SAUE15-N6 | TSPE4-B2 | SAUE15-K10 | SAUE15-N6 | TSPE4-H6 | TSPE4-F6 | SAUE15-L4 | SAUE15-L11 |
| A n = 16 | 0 | 0 | 0 | 0 | 0 | 0 | 0 | 0 |
| B1 n = 16 | 0 | 3 | 0 | 0 | 0 | 0 | 0 | 0 |
| | | (18.75%) | | | | | | |
| B2 n = 115 | 13 | 11 | 9 | 2 | 2 | 4 | 7 | 15 |
| | (86.67%) | (73.55%) | (60%) | (13.33%) | (13.33%) | (26.67%) | (46.67%) | (100%) |
| D n = 12 | 0 | 0 | 0 | 0 | | 0 | 0 | 6 |
| | | | | | | | | (50%) |

| ECOR | PREVALENCE OF THE SUBTRACTIVE CLONES WITH NO HOMOLOGY, | | | | | |
|---|---|---|---|---|---|---|
| COLLECTION | IN THE STRAINS OF THE ECOR COLLECTION | | | | | |
| GROUPS | SAUE15-M10 | TSPE4-C2 | TSPE4-E7 | TSPE4-D8 | SAUE15-N4 | SAUE15-I12 |
| A n = 16 | 0 | 0 | 2 | 0 | 0 | 0 |
| | | | (12.5%) | | | |
| B1 n = 16 | 0 | 15 | 0 | 1 | 0 | 3 |
| | | (93.75%) | | (6.25%) | | (18.75%) |
| B2 n = 115 | 15 | 12 | 12 | 10 | 7 | 15 |
| | (100%) | (80%) | (80%) | (66.67%) | (46.67%) | (100%) |
| D n = 12 | 3 | 2 | 3 | 0 | 1 | 12 |
| | (25%) | (16.67%) | (25%) | | (8.33%) | (100%) |

Between brackets: Frequency of the clone among the ECOR group under consideration (percentage of strains of this ECOR group in which the clone is present).

It is observed that the vast majority of the fragments tested (more than 90%) are absent (frequency of presence 0%) from the 16 E. coli strains of group A which the ECOR collection contains, and that they are all present at a frequency greater than 10% in the 15 E. coli strains of group B2 which the ECOR collection contains. More than 75% of the fragments are present in the 15 ECOR E. coli strains of group B2 at a frequency greater than 40%, 50% of them are present therein at a frequency greater than 70% and approximately 35% of them are present therein at a frequency greater than or equal to 80%.

It is also observed that some of these fragments are also present in ECOR E. coli strains of groups B1 and/or in ECOR E. coli strains of group D. It is in particular noted that the TspE4.C2 clone (SEQ ID NO: 119) is present in the ECOR E. coli of group B1 at a frequency greater than 90%, while at the same time being completely absent from the ECOR E. coli of group A. The SauE15.12 clone (SEQ ID NO: 37) is, itself, present with a frequency of 100% in the ECOR E. coli of group D and with a frequency of 100% in the ECOR E. coli of group B2, while at the same time being completely absent from the ECOR E. coli of group A and barely present in the ECOR E. coli of group B1.

All the fragments tested herein have in common the fact that they are present at a frequency greater than 10% in the ECOR E. coli of group B2 and at a frequency of less than 25%, particularly less than 10% and notably less than 5% in the ECOR E. coli of group A.

Since the ECOR collection represents the genetic diversity of the E. coli species, the various results obtained indicate that the set of DNAs isolated according to the invention constitute, taken alone or in combination, particularly suitable tools for the phylogenic identification of E. coli strains.

In order to further refine knowledge of the phylogenic distribution of the isolated fragments, the epidemiological study was pursued for several other C5+ A− clones. The results are given in Table 4 below (groups A, B1, B2 and D, group X including the 4 strains of the ECOR collection which are not assigned to any of these 4 groups).

TABLE 4

| | | ECOR | | | (n = 72) | |
|---|---|---|---|---|---|---|
| | SEQ ID | A | B1 | B2 | D | X |
| Clone | NO | n = 25 | n = 16 | n = 15 | n = 12 | n = 4 |
| SauE15.B10 | 174 | 4 | 0 | 0 | 41.7 | 0 |
| SauE4.A2 | 202 | 4 | 0 | 0 | 41.7 | 0 |
| SauE15.C7 | 178 | 4 | 0 | 0 | 41.7 | 0 |
| | 221 | 4 | 0 | 0 | 41.7 | 0 |
| TspE4.B9 | | | | | | |
| TspE15.G6 | 102 | 12 | 0 | 53.3 | 83.3 | 0 |
| SauE4.C11 | 206 | 0 | 0 | 66.7 | 0 | 0 |
| SauE4.E6 | 71 | 0 | 0 | 66.7 | 0 | 0 |
| TspE4.A11 | 114 | | | | | |
| SauE15.C12 | 13 | 0 | 0 | 100 | 66.7 | 0 |
| SauE4.G11 | 77 | | | | | |
| SauE15.A12 | 8 | 0 | 0 | 93.3 | 16.7 | 0 |
| SauE15.B12 | 175 | 0 | 12.5 | 93.3 | 0 | 0 |
| SauE15.J7 | 196 | 0 | 18.75 | 73.3 | 0 | 50 |
| SauE15.A7 | 170 | | | | | |
| SauE15.I8 | 36 | 0 | 0 | 13.3 | 0 | 0 |
| TspE4.C3 | 120 | 24 | 43.75 | 86.7 | 66.7 | 25 |
| TspE4.F6 | 130 | 0 | 0 | 26.7 | 0 | 0 |

It can be observed that, as for the fragments previously tested, the majority of the fragments are present at a frequency greater than 10% in the E. coli of group B2 and at a frequency of less than 10% in the E. coli of group A. It can in particular be noted that the SauE15.C12, SauE15.A12 and SauE15.B2 clones are present at a frequency of 100%, 93.3% and 93.3%, respectively, in the ECOR E. coli of group B2, and that all three of them are present at a frequency of 0% in the ECOR E. coli of group A.

One clone, TspE15.G6, is however present at a frequency of 12% in the E. coli of group A, at a frequency of 53.3% in the ECOR E. coli of group B2, and at a frequency of 83.3% in the ECOR E. coli of group D.

Four other clones, namely SauE15.B10, SauE4.A2, SauE15.C7 and TspE4.B9 appear, themselves, not to be present in the ECOR E. coli of group B2; these clones are, however, present at a frequency greater than 10% in the ECOR E. coli of group D (41.7%), and at a frequency of less than 10% in the ECOR E. coli of group A (4%).

The choice of the E. coli strain C5 as the strain of group B2 for the subtractive hybridization which enabled the isolation of these fragments (cf. Example 1 above) is probably not unrelated to these results. The E. coli strain C5, although belonging to the phylogenic group B2, in fact comprises a plasmid some sequences of which are also present in E. coli of group D (frequency of 41%).

The choice of such a strain for isolating the set of fragments which are very generally absent from the ECOR E. coli of group A therefore makes it possible to isolate the entire set of fragments which are present with greater frequency in the ECOR E. coli of group B2 and/or D, with respect to the ECOR E. coli of group A. The majority of the fragments tested are, moreover, completely absent from the ECOR E. coli of group A. When their frequency of presence among the ECOR E. coli of group A is not zero, it remains low (maximum measured for the fragments of this example=24%), and it is always less, by a factor of approximately 3 or 4, than the frequency observed either in the ECOR E. coli of group B2 or in the ECOR E. coli of group D, or in each of them.

Since the extra-intestinal pathogenicity of E. coli is associated with strains of group B2 or D, and not with strains of group A, the fragments isolated in accordance with the invention using a B2/D E. coli strain such as E. coli C5 have, in addition to phylogenic diagnostic applications (cf. Example 5 below) , applications of particular value for preventing, alleviating and combating any extra-intestinal development of E. coli (systemic and nondiarrhoeal development).

This being so, it will become clearly apparent to those skilled in the art that the examples reported herein with the E. coli strain C5 can be carried out in a similar manner with another E. coli strain of B2/D type, such as RS218, such as E. coli CFT073, and/or with a D strain.

In conclusion, the results obtained show that the C5+ A− fragments isolated in accordance with the present invention are present in the ECOR E. coli of group A at a frequency lower than that which can be observed in the ECOR E. coli of group B2 and/or in the ECOR E. coli of group D (=nature B2/D+ A−).

Their frequency of presence in the ECOR E. coli of group A is generally zero; if this is not the case, it is lower than that observed in the ECOR E. coli of group B2 and/or D by a factor which is at the very least 2, preferably 3, more preferably 3.5, and very preferably 4. This B2/D+ A− set according to the invention in particular comprises mostly fragments which are present at a frequency greater than 10% in the ECOR E. coli of group B2 and/or D, and at a frequency of less than 25%, preferably 20%, more preferably 10%, and even more preferably 5%, in the ECOR E. coli of group A (with the proviso that this A frequency is always lower than that in the B2s and/or Ds).

The present invention also provides means which give access to the entire set of these polynucleotides which are of nature B2/D+ A−.

### EXAMPLE 3

Example of Medical Application of the B2/D+ A− Fragments Isolated (Systemic and Non-diarrhoeal Development of E. coli in an Extra-intestinal Compartment)

Since the B2/D+ A− E.coli are those which are particularly responsible for extra-intestinal infections, we attempted to determine whether some of the B2/D+ A− fragments thus isolated were, in fact, involved in a step essential to the extra-intestinal infectious process of E. coli, namely survival and multiplication in the blood.

The approach used is that of differential transcriptional analysis (DTA) which consists in revealing the transcripts induced during this step. In order to also determine which characteristics of the serum are responsible for the variation of the level of gene expression of E. coli, the DTA was carried out under the following growth conditions:

the nutrient broth constitutes the control,

the bacteraemia phase is compared to the growth of the bacterium in the presence of human serum,

the iron deficiency which the culturing in serum induces is reproduced using a culture in nutrient broth supplemented with an iron chelator,

the effect of the complement is studied using growth in the presence of decomplemented serum.

Comparison of the transcriptomes obtained under each of these culture conditions makes it possible to reveal the genes specifically involved in the growth in serum, and to produce a functional group of the genes subjected to the same regulatory factor, such as iron content or stress induced by the bactericidal activity of the serum.

Materials and Methods

1. Bacterial Strains, Culture Media and Subtractive Clones

The E. coli strain C5 (serotype O18:K1:H7) was isolated from the CSF of a newborn. This virulent strain belongs to the phylogenic group B2 and exhibits the following virulent factors: The K1 capsular polysaccharide, S adhesin, Ibe10 invasin, the type P pilus and haemolysin, but does not produce aerobactin. The subtractive fragment library was obtained using the C5 strain. The nonpathogenic strains used were the E. coli K12 strain MG1655 and the strain ECOR15 belonging to the so-called nonpathogenic phylogenic group A, originating from the ECOR reference collection.

The bacterial inocula were prepared from 18 h cultures on tryptocasein-soybean agar (Sanofi Pasteur), with the colonies being resuspended in sterile water. After measuring the OD, this bacterial suspension, pure or diluted, was used to prepare the inoculum in the various culture media, adding a volume which was always less than 1/10th of the final volume.

The bacterial cultures were prepared at 37° C. with shaking, using either pure nutrient broth (Sanofi Pasteur) or nutrient broth supplemented with an iron chelator: 2,2′-dipyridyl (Sigma) at a concentration of 200 μM. The bacterial strains were also cultured in the presence of human serum. Two types of serum were obtained: one consisting of a pool of 4 sera originating from donor blood, the other corresponding to a single donor. The serum was collected after harvesting the blood in a dry tube, spontaneous coagulation for approximately 3 hours at room temperature, and then decanting. The serum was then stored in the form of 2.5 ml aliquots at −80° C. Decomplemented serum was obtained, from the pool, after incubation at 56° C. for 30 minutes.

The subcultures of the clones of the B2/D+ A− library were prepared in nutrient broth enriched with ampicillin (50 μg/ml) at 37° C.

2. Study of the Bactericidal Effect of the Serum

In order to evaluate the resistance of the E. coli strain C5 to the bactericidal effect of the serum, on the one hand, and the intact activity of the complement in the serum, on the other hand, growth curves were produced. The bacterial growth was analysed by taking various inocula of the strains ECOR15, K12 and C5, and carrying out bacterial counts of cultures in pure serum or in decomplemented serum. These counts were taken at times corresponding to 0 h, 3 h, 6 h and 24 h, by plating out pure or serially diluted cultures on Petri dishes using a “spiral meter” system.

3. Amplification of the Subtractive DNA Fragments Specific for E. coli K1 and Manufacture of the High Density Membranes

a. PCR of the Subtractive DNA Fragments

The subtractive DNA fragments cloned into the plasmid pUC19 were amplified by PCR reaction, without DNA extraction, directly from a 1/10th dilution of an 18 h culture broth of each clone. 5 μl of this bacterial solution were added to the reaction mixture, with a final volume of 50 μl, comprising: 1×PCR buffer (10 mM Tris-HCl pH 9; 1.5 mM MgCl2; 50 mM KCl; 1% Triton X100; 0.1% gelatin); 2U SuperTaq polymerase (ATCG Biotechnologie); 200 μM deoxynucleotide triphosphates; 6 μM specific primers. The primers used were as follows: P1 (5′-CATGCCTGCAGGTCGACTCT-3′; SEQ ID NO: 728), P2 (5′-CGTTGTAAAACGACGGCCAG-3′; SEQ ID NO: 729). The PCR reaction consisted of 30 cycles: 30 s at 95° C. (denaturation), 30 s at 55° C. (hybridization) and 30 s at 72° C. (elongation).

b. Preparation of the High Density Membranes

Membranes comprising the set of specific amplified fragments of the E. coli strain C5 were manufactured by the company Eurogentec. 180 nl of each of the PCR products were deposited in duplicate, in the form of microspots, onto 6 cm by 11 cm nylon membranes. In order to be able to normalize the signals recorded for each reverse transcript, spots consisting of 4-fold serial dilutions of the chromosomal DNA of the E. coli strain C5, on the one hand, and of dilutions of the product of a PCR of the 16S rRNA, on the other hand, were deposited. In addition, a negative control corresponding to the PCR product of a subtractive fragment of 17 bp was also deposited. These membranes are conserved sealed, at +4° C.

4. Synthesis of the ³³P-labelled Reverse Transcripts

a. Extraction of the Total RNA

In order to prevent degradation of the RNA by RNAses, all the extraction steps were carried out in ice, using gloves and RNAse-free material. The RNA is extracted from a bacterial pellet obtained after centrifugation (4 700 rpm at 4° C. for 3 min) of 5 ml of a 4-hour culture (approximately 10⁸ CFU/ml).

For the purpose of evaluating the influence of the extraction method, two techniques were used to extract the total RNA: the TRIZOL reagent and the BIORAD kit.

TRIZOL reagent (Gibco BRL): this reagent consists of a monophasic solution of phenol and of guanidine isothiocyanate, which allows extraction of the total RNA in a single step according to the method developed by Chomczynski and Sacchi. The bacterial cells are lysed by adding the TRIZOL reagent, vortexed for 30 s and subjected to a heat shock (incubation at 65° C. followed by freezing at −80° C.). The following steps are those of a conventional phenol-chloroform extraction. Finally, the recovered aqueous phase contains the RNA.

BIORAD kit: this kit contains no phenol-based solution. It consists in using a solution which allows the cells to be lysed during an incubation at 65° C. for 5 minutes. Next, a solution for precipitating the DNA and the proteins makes it possible to then recover the RNA which is in the aqueous phase.

For these two methods, the subsequent steps of precipitation and solubilization of the RNA are identical. The RNA is precipitated using isopropanol: the isopropanol is added to the aqueous solution containing the RNA, volume for volume. This mixture is incubated at −20° C. for approximately 15 h, and after centrifugation (13 000 rpm at 4° C. for 5 min), the RNA is obtained in the form of a pellet. The RNA is then washed with 70% ethanol and precipitated by centrifugation (13 000 rpm at 4° C. for 2 min). Finally, the RNA is solubilized in water in the first case (TRIZOL) or in a rehydration solution in the second case (BIORAD). The RNA samples are stored at −80° C.

b. Analysis of the RNA Samples Obtained

RNA Assay and Purity

The RNA is assayed using a spectophotometer, by measuring the absorbance at 260 nm, it being known that an optical density value at 260 nm corresponds to an RNA concentration of 40 μg/ml. The purity of the sample is estimated by calculating the OD_{260 nm}/OD_{280 nm} ratio: a ratio greater than 1.6 reflects an acceptable purity.

RNA Quality

Approximately 0.3 μg of RNA is analysed by 2% agarose gel electrophoresis—80 volts. The image obtained allows mainly the visualization of the bands corresponding to the 23S rRNA and the 16S rRNA, and a band corresponding to the 5S RNA and to the transfer RNAs (cf. figure), if the RNA preparation obtained is of good quality.

c. Synthesis of the ³³P-labelled cDNA Probe

The cDNA probe is synthesized using random priming. 10 μg of RNA are mixed together, in a final volume of 50 μl, with 10 μg of random hexamers; dATP, dTTP and dGTP, final concentration of 10 μM (Boehringer Mannheim); 40 U of Rnasin ribonuclease inhibitor (Promega); 1×RT buffer; 10 μg of bovine serum albumin (Promega). This reaction mixture is incubated at 50° C. for 5 minutes so as to linearize the mRNAs, and then brought back to +4° C. in ice. Finally, 100 U of M-MuLV Reverse Transcriptase (New England Biolabs) and 50 μCi of [α-33P]dCTP (Amersham Pharmacia Biotech) are added. Finally, this reaction mixture is incubated at 37° C. for 2 hours.

5. Molecular Hybridization

Prehybridization

Initially, the membrane is prehybridized in 5 ml of Church and Gilbert hybridization buffer (0.5 M NaPi, pH 7.2; 1 mM EDTA; 0.7% SDS) for a minimum of 30 minutes at 65° C.

Hybridization

After having been denatured (5 min at 95° C.) , the ³³P-labelled cDNA probe is directly added to 5 ml of hybridization buffer. The hybridization reaction is carried out at 65° C. for 15 to 18 h.

Washing

The membrane is first rinsed twice with washing buffer (40 mM NaPi, pH 7.2; 1 mM EDTA; 1% SDS). Then, four washing steps are carried out (30 min at 65° C.).

Exposure

Once wrapped in Saran (food film-wrap), the membrane is exposed to a ³³P-sensitive screen (Molecular Dynamics) for 48 h to 90 h.

Dehybridization

The membrane is incubated twice for 15 min at 37° C. in the presence of a solution of 0.2N NaOH and 0.1% SDS, and then it is rinsed with distilled water for 5 min.

6. Analysis of the Transcriptomes

Data Acquisition

The exposed ³³P-sensitive screen is scanned using a PhosphorImager (Storm 840, Molecular Dynamics) with a pixel size of 50 μm, and then the image obtained is analysed using the XdotsReader software (Cose). This software allows automatic recognition of the spots and is capable of calculating the pixel-density for each of the spots. It also makes it possible to subtract the background noise and to normalize the signal intensity. For each spot, the local background noise was subtracted and the signal intensity was normalized using the signal of the chromosome diluted 400-fold.

Data Analysis

First, the disparity between two spots from the same clone was calculated in order to eliminate the spots with a significant disparity, reflecting aberrant signals. Then, the mean of the intensity of the signals from a same pair was defined, and it is this mean intensity, net (after subtraction of the local background noise) and normalized, which will be considered for the data analysis. The intensity obtained for the 17 bp clone made it possible to define a positivity threshold, the signals lower than this intensity being considered as negative or “undetected”.

Results

1. Validation of the Model for Studying the Bactericidal Effect of the Serum

Ability of E. coli C5 to multiply in serum. The growth curves produced with an inoculum of 10⁷ CFU/ml for the E. coli strain C5 responsible for neonatal meningitis, and the nonpathogenic E. coli strains K12 and ECOR15, show that, in the presence of human serum, E. coli C5 is capable of surviving and of multiplying, whereas as E. coli strain K12 is killed in less than 2 hours and the strain ECOR15 experiences a decrease in growth of more than 2 logs in 2 hours, persisting at a level of between 10⁴ and 10⁵ CFU/ml. At a lower inoculum (10³ or 10⁵ CFU/ml), the strain C5 persists without growing for the first two hours of culturing, and then experiences a growth of 1 log in the 4th hour. The inoculum of 10⁷ CFU/ml was therefore selected for the transcriptome study.

In decomplemented serum, the growth of the strains K12 and ECOR15 is similar to that of the strain C5, which suggests that the bactericidal effect observed was due to the lytic activity of the complement.

In order to determine whether or not the survival of the strain C5 between 2 h and 6 h of culturing was due to the modification of the complement at 37° C., growth curves for the strain K12, in the presence of serum incubated beforehand at 37° C. for 2 h, 4 h and 6 h, were produced. The results of this experiment demonstrate that the serum still possesses its bactericidal activity, even after having been pre-incubated for 6 h at 37° C.

2. Isolation of the RNAs and Comparison of the Two Extraction Methods

Since the RNA extraction step is a fundamental step of the DTA, it appeared to us to be necessary to compare two extraction methods in order to determine whether or not the mode of extraction could have an influence on the transcriptome results. Preparations of good quality are characterized by the presence of the 23S, 16S and 5S rRNAs, detected in the form of clear bands by agarose gel electrophoresis. The RNAs extracted with the Biorad kit and the Trizol reagent, from a nutrient broth culture supplemented with dipyridyl, were analysed on 2% agarose gel under nondenaturing conditions. The bands obtained for the 23S and 16S rRNAs, and a band corresponding to the 5S rRNA and to the tRNAs reflect the good quality of the RNA preparations and of the equivalence of the extraction methods. In addition, the detection of RNA of low molecular weight shows that the extraction method makes it possible to isolate the mRNAs which are small in size, unlike other systems and in particular those using columns which retain only the mRNAs longer than 200 bp. However, a high molecular weight band corresponding to the chromosomal DNA appears clearly on the lane of the RNA obtained using the Biorad kit. The two transcriptomes obtained from these two types of RNA were compared visually (without integrating the signals), and appear to be identical. These results suggest the absence of an influence of the extraction methods and, in particular, of the contaminant chromosomal DNA. The latter point was, moreover, confirmed by carrying out a comparison between a probe obtained using Biorad RNA, with and without DNAse treatment.

3. Differential Expression of the Transcripts Corresponding to the DNA Fragments Specific for E. coli K1 Responsible for Neonatal Meningitis

The transcriptomes obtained by hybridization of the reverse transcripts originating from E. coli C5, under two different culture conditions, on the high density membranes comprising the set of clones of the B2/D+ A− library were analysed with the aid of the Cose software, using the 400-fold dilution of the chromosome as the normalization spot, the signal of which is close to the median of all of the signals. It appears clearly that a certain number of spots are lacking in signals. In order to determine objectively the reverse transcripts which will be considered to be negative or “undetected”, the normalized intensity recorded on the 17 bp clone (0.05) was used. In order to be safe, this threshold was doubled and, therefore, any spot with an intensity of less than 0.1 was considered to be “undetected”.

The normalized intensities of the signals obtained for all the clones for which the transcript was detected under at least one of the culturing conditions make it possible to visualize the level of transcription of the set of subtractive clones in the course of the three respective experimental conditions: growth in nutrient broth, in nutrient broth supplemented with 2,2′-dipyridyl (iron chelator) and in serum. It is noted that most of the signals detected have similar intensities whatever the culturing conditions, whereas certain fragments exhibit levels of transcription which vary by a factor of ten according to the culturing conditions. These results therefore suggest good reproducibility of the technique, with a capacity to detect different transcriptional levels.

The transcriptome obtained in nutrient broth was considered to reflect the basal level of transcription of the bacterium in favourable medium. The expression profile obtained under conditions of stress consisting of culturing in serum was analysed with respect to this control transcriptome. In addition, the transcriptomes obtained when culturing in nutrient broth and iron chelator were also prepared, in order to determine the respective roles of iron deficiency and of complement in the serum.

Table 5 below gives the ratios of the intensities of the signals obtained for some of the clones of the B2/D+ A− library under the various experimental conditions, with respect to the control condition represented by the growth in nutrient broth (NB). Overall, it appears that the transcripts induced in the serum (ser) are most commonly also induced in the presence of dipyridyl (dip) and of decomplemented serum (DC serum), with the exception of the SauE15.A12, SauE4.E6, SauE4.C11 and TspE15.G6 clones.

It is interesting to note that these four clones have transcripts induced by serum factors other than iron deficiency, since their level of transcription is not modified, or even decreased, in the presence of dipyridyl (cf. Table 5). The transcription of two of these clones (SauE15.A12 and SauE4.C11) is not induced in decomplemented serum, and they therefore represent genes which are excellent candidates for complement resistance.

TABLE 5

| | | RATIOS | | |
|---|---|---|---|---|
| Clone | SEQ ID NO: | ser/NB | dip/NB | DC serum/NB |
| SauE15.B10 | 174 | 3.58 | 2.04 | 2.72 |
| SauE4.A2 | 202 | 10.58 | 13.09 | 10.57 |
| SauE15.C7 | 178 | 12 | 3.86 | 9.58 |
| TspE4.B9 | 221 | 6.03 | 2.72 | 7.57 |
| TspE15.G6 | 102 | 2.22 | 0.08 | 1.72 |
| SauE4.C11 | 206 | 2.46 | 0.84 | 1.25 |
| SauE4.E6 | 71 | 3.27 | 0.44 | 2.76 |
| TspE4.A11 | 114 | 1.82 | 0.74 | 1.54 |
| SauE15.C12 | 13 | 2.65 | 3.35 | 3.34 |
| SauE4.G11 | 77 | 1.78 | 1.09 | 1.07 |
| SauE15.A12 | 8 | 4.58 | 1.08 | 1.26 |
| SauE15.B12 | 175 | 2.81 | 1.77 | 1.62 |
| SauE15.J7 | 196 | 3.06 | 1.71 | 3.14 |
| SauE15.A7 | 170 | 17.45 | 6.62 | 14.82 |
| SauE15.I8 | 36 | 1.99 | 0.11 | 1.24 |
| TspE4.C3 | 120 | 1.96 | 2.26 | 1.39 |
| TspE4.F6 | 130 | 2.22 | 0.69 | 1.45 |

Reproducibility:

In order to verify the reproducibility of the technique and to demonstrate that the differences in transcription level detected are not linked to factors specific to the pool of serum used, a new probe was prepared from a culture of E. coli C5 in a serum originating from a single donor. The transcriptome obtained with this new probe was compared with the transcriptome produced with the pool consisting of several sera. The straight line of regression obtained, and also the regression coefficient with a value of 0.85, indicate the excellent reproducibility of the technique.

Relationship Between the Normalized Intensity and the Amount of Reverse Transcripts:

With the aim of verifying that there is indeed a linearity between the normalized intensity and the amount of reverse transcripts, the intensities obtained for the chromosomal range points consisting of 1/100th, 1/400th and 1/1600th dilutions were recorded on a graph. The graph obtained shows the existence of a linear relationship between these values, making it possible to deduce an induction factor directly from the normalized intensities recorded.

Conclusion

It appears, therefore, that the DTA technique described herein is a reliable method for selecting, from the B2/D+ A− library obtained from E. coli C5, DNA fragments the transcription of which is increased in the presence of serum. These fragments make up genes which participate specifically in the systemic and non-diarrhoeal extra-intestinal development of E. coli in humans and animals. These genes can be isolated using said DNA fragments the transcription of which is increased in the presence of serum, according to conventional techniques of those skilled in the art (cf. Example 6).

These fragments and the genes which bear them can be used as active principles (in the form of naked DNA placed under the control of a eukaryotic promoter or in the form of DNA transfected into a cell) in a vaccine composition intended to prevent, alleviate or combat the systemic and non-diarrhoeal development of E. coli in a human or animal extra-intestinal compartment. For this purpose, these fragments and genes can, if desired, be modified, for example so as to produce inactivated isogenic mutants.

The polypeptides which they encode can also be used in such vaccine compositions, in an inactivated and immunogenic form.

These DNA fragments and these genes can also be used as anti-pathogenicity targets (with the objective of preventing the development of E. coli in an extra-intestinal area, and not in the intra-intestinal area): they allow the identification of compounds capable of specifically inhibiting their transcription and/or translation, or of compounds capable of inhibiting the activity of the proteins encoded by these genes. Such compounds can be used as active principles in pharmaceutical compositions (medicinal products) in order to prevent, alleviate or treat the systemic non-diarrhoeal development of E. coli in a human or animal extra-intestinal compartment.

Among the fragments which are of nature B2/D+ A−, and the transcripts of which are increased in the serum, those which are not present in E. coli which are agents of infections localized in the intestine, such as E. coli O157:H7, are more particularly preferred.

A systemic non-diarrhoeal development of E. coli in an extra-intestinal compartment is in particular observed in the context of diseases such as neonatal meningitis, septiceamias, sepsis or pyelonephritis. Such vaccines and pharmaceutical compositions are particularly useful in the context of hospital-acquired infections. Such vaccines are most particularly valuable for the vaccination of women (from adolescent to adult, and more particularly before and during a gestation period) as a prevention against pyelonephritis, in order to avoid contamination of the newborn during birth.

The vaccines and pharmaceutical compositions according to the invention are therefore particularly suitable for such pathologies.

The present invention is therefore aimed towards any polynucleotide capable of being obtained:

by subtractive hybridization of an E. coli strain of group B2 or D against one or more E. coli strains of group A,

isolation of the substraction DNA fragments, and

selection of those for which transcription is stimulated in the presence of serum, with respect to a standard nutrient medium.

The DNAs given in Table 5 above are examples of such polynucleotides.

The present application is thus aimed towards:

any vaccine composition comprising such a polynucleotide, or an inactivated isogenic mutant of such a polynucleotide (inactivation of the possible pathogenic potency), in particular for preventing, alleviating or combating the development (systemic and non-diarrhoeal) of E. coli in a human or animal extra-intestinal compartment,

any pharmaceutical composition comprising a compound capable of inhibiting the transcription and/or translation of such a polynucleotide or mutant, or capable of inhibiting the activity of a polypeptide encoded by such a polynucleotide or mutant.

The present invention is also aimed towards a method for identifying compounds which can be used as active principles in a pharmaceutical composition (medicinal product) intended to prevent, alleviate or combat the development (systemic and non-diarrhoeal) of E. coli in a human or animal extra-intestinal compartment. This method comprises detecting and selecting compounds capable of inhibiting the transcription and/or translation of said polynucleotides, or capable of inhibiting the activity of the polypeptides which they encode. The present invention is also aimed towards any kit suitable for the implementation of this method, said kits comprising at least one of said polynucleotides or polypeptides. Any transgenic cell and any non-human transgenic animal, into which at least one of said polynucleotides or inactivated isogenic mutant has been transfected, also enter into the domain of the present application. Such cells and transgenic animals are particularly useful for selecting active principles of interest.

### EXAMPLE 4

Production of the Sequences of the B2/D+ A− Regions

In Example 1, the isolation and the sequence of 259 DNA fragments which are of nature B2/D+ A− (153 of which are novel as products) are described, and means for producing the entire set of these products are provided.

Those skilled in the art will appreciate that, using these DNA fragments, the sequence of each of regions 1, 2, 3, 4, 5, 6a and 6b described, and ORFs which correspond to them, can be identified, isolated and sequenced.

Example 1 and FIG. 1 give the sequence of eight (novel) fragments belonging to region 1 (SEQ ID NO: 134, 144, 109, 115, 140, 135, 33 and 56). For region 2, 5 novel fragments (SEQ ID NO: 125, 123, 116, 43 and 40), and the presence of the sfa gene, are indicated. For region 3, 7 (novel) fragments (SEQ ID NO: 122, 130, 141, 25, 48, 51 and 57) are indicated. For region 4, three (novel) fragments (SEQ ID NO: 121, 44 and 45) are indicated. For region 5, 5 (novel) fragments (SEQ ID NO: 113, 119, 120, 23 and 52) are indicated. For region 6a, eight novel fragments (SEQ ID NO: 127, 133, 27, 34, 36, 42, 46 and 54), and the presence of the known ibe10 product, are indicated. For region 6b, four (novel) fragments (SEQ ID NO: 55, 38, 128 and 151), and the presence of four known products (SEQ ID NO: 212, 226, 201 and 229), are indicated.

The provision of the sequences allows those skilled in the art to obtain the complete sequence of the corresponding region, according to conventional techniques, and to identify, in these regions, the presence of possible open reading frames (ORFs).

One of the conventional techniques consists in developing primers for amplifying the desired region, based on the sequences of the fragments of this region, and in carrying out a PCR amplification using various combinations of said primers placed in contact with the polynucleotide population of an E. coli strain of group B2 or D (which may or not be ECOR), under conventional PCR conditions. The PCR products which overlap are sequenced on both strands, using the chain termination technique and automated sequencing.

If necessary, the sequence obtained can be extended beyond the limit of the clones available by cloning, for example in lambda DASH-II, partial fragments of approximately 15 kb obtained by restriction carried out on said polynucleotide population. The inserts overlapping the desired region are then identified by hybridization with clones of this region. The inserted DNA is then sequenced from the end of the inserts, and these sequences are used to develop novel primers which will be used to directly amplify the chromosomal (and non-phage) DNA. Amplification of the chromosomal DNA is then obtained using these novel primers and those of the shorter sequence already obtained. These PCR strands are also sequenced on both strands, which then produces the complete sequence of the desired region.

Alternatively, the sequence of such regions can be obtained by extension of the sequencing of the chromosome of an E. coli strain of group B2 or D, from points at which a clone which is of nature B2/D+ A− is located (cf. Example 6 below).

The open reading frames of the sequences obtained for the regions can then be analysed according to conventional techniques for seeking ORFs. A search is carried out in particular for ORFs which begin with ATG or CTG and which have a high codon use index.

### EXAMPLE 5

Method and Kit for Identifying the Phylogenic Group of an E. coli Strain

As indicated in Examples 1, 2 and 3, the polynucleotides which are of nature B2/D+ A− can be used to determine the phylogenic group of any E. coli strain. They can be used alone, in combination together or in combination with other products, depending on the result and the precision of phylogeny desired. In the event of positive detection, the set of DNAs which are of nature B2/D+ A− makes it possible to eliminate the hypothesis that a strain belongs to the group A. More finely, the presence or absence of the chuA gene, or of the fragments which are of nature B2/D+ A− (in particular SEQ ID NO: 241, 195, 185 and 248), each make it possible to completely distinguish between group A or B1, on the one hand, and group B2 or D, on the other hand. The presence or absence of the TspE4.C2 fragment (SEQ ID NO: 119), of the gene which corresponds to it or of the other fragments, of this gene, which are of nature B2/D+ A− make it possible to completely distinguish between group A and group Bl. The presence or absence of the SauE15.I12 fragment (SEQ ID NO: 37), of the gene which corresponds to it or of the other fragments, of this gene, which are of nature B2/D+ A− make it possible to completely distinguish between group B2 or D and group A. The detection of such presences or absences can take place by any means available to those skilled in the art. It can in particular be carried out using said polynucleotides as probes (Southern technique), or by constructing amplification primers capable of amplifying one of said polynucleotides, so as to carry out a PCR. The construction of probes and of primers can be carried out according to any technique known to those skilled in the art; examples of such constructions are given below. When these polynucleotides are coding polynucleotides, the detection of the corresponding polypeptides (using antibodies directed against these polypeptides) constitutes a variant of implementation.

Using the teaching given in the present application, those skilled in the art can design the decision tree which corresponds to the level of phylogenic precision desired.

More particularly described here, is an example of a phylogenic identification method which allows a phylogenic precision of at least 99% for E. coli. This method is based on the PCR detection of two genes (chuA and yjaA), the sequence of which is known, but which are of novel nature, and on the detection of the TspE4.C2 novel DNA fragment (SEQ ID NO: 119). The method was evaluated by testing 220 strains which had already been grouped together using reference methods (multilocus enzymatic electrophoresis, MLEE and/or ribotyping). The inventors in fact demonstrated that the known chuA gene is present in 100% of the ECOR strains of group D, in 100% of the ECOR strains of group B2, and in 0% of the ECOR strains of group A and of the ECOR strains of group B1. They also demonstrated that the yjaA gene, the sequence of which is known, is present in 100% of the ECOR B2 strains and in 0% of the ECOR strains of group D. The yjaA gene was, until then, only known to be present in the E. coli strain K12 (group A), but had no known function. It was also demonstrated that the TspE4.C2 novel fragment is present in approximately 94% of the ECOR strains of group B1 and in 0% of the ECOR strains of group A. The combination of these three phylogenic markers makes it possible to access a level of effectiveness of distinction between the groups A, B1, B2, B2 and D, which is greater than 99%. A technical procedure for implementing this combination is also described, which is technically very advantageous: triplex PCR. This novel method is rapid and simple, it can be used directly on a bacterial colony, and it does not require having a reference collection, unlike the techniques of the prior art, and MLEE and ribotyping in particular. The method described therefore represents the first method which may constitute a real clinical tool for routine analyses.

Materials and Methods

Bacterial strains. The 72 strains of the ECOR collection are available from the ATCC. These reference strains, isolated from various hosts and various geographical locations, are representative of the range of genotypic variation of the species. Sixty-eight of these strains belong to the four main phylogenic groups (A, B1, B2 and D), and 4 are unclassified.

A set of 86 E. coli strains having caused neonatal meningitis (NMEC), 34 E. coli strains responsible for neonatal septicaemia without meningitis, 30 E. coli strains isolated from healthy newborns, and the uropathogenic E. coli strain J96 (Q4:K6) were also tested. The distribution by phylogenic group of 69 of the 86 NMEC strains has already been described (Binger et al. 1998, J. Infect. Dis. 177:642-650). The other 17 NMEC and the remaining 65 clinical isolates were classified by means of ribotyping as previously described in Binger et al. (ref. above). The laboratory E. coli K-12 strain MG1655, which belongs to the phylogenic group A, was also used.

The bacteria were cultured at 37° C. on Luria Bertani broth medium or agar. If necessary, ampicillin (100 μg per ml) was used.

PCR amplification. In a first step, the PCR was carried out according to a standard protocol. The reaction was carried out in a volume of 20 μl containing 2 μl of 10×buffer (supplied with Taq polymerase), 20 pmol of each primer, 2 μM of each dNTP, 2.5 U of Taq polymerase (ATGC Biotechnologie, Noisy-le-Grand, France) and 200 ng of genomic DNA. The PCR was carried out using a Perkin-Elmer GeneAmp 9600 thermal cycling machine, with MicroAm tubes, under the following conditions: denaturation for 5 minutes at 94° C., 30 cycles of 30 seconds at 94° C., 30 seconds at 55° C. and 30 seconds at 72° C., and a final extension step of 7 minutes at 72° C., using the pairs of primers:

| | |
|---|---|
| chuA.1 | |
| (5′-GACGAACCAACGGTCAGGAT-3′; and | SEQ ID NO: 160) |
| chuA.2 | |
| (5′-TGCCGCCAGTACCAAAGACA-3′;, | SEQ ID NO: 161) |
| for chuA | |
| yjaA.1 | |
| (5′-TGAAGTGTCAGGAGACGCTG-3′; and | SEQ ID NO: 162) |
| yjaA.2 | |
| (5′-ATGGAGAATGCGTTCCTCAAC-3′;, | SEQ ID NO: 163) |
| for yjaA and | |
| TspE4C2.1 | |
| (5′-GAGTAATGTCGGGGCATTCA-3′; and | SEQ ID NO: 164) |
| TspE4C2.2 | |
| (5′-CGCGCCAACAAAGTATTACG-3′; | SEQ ID NO: 165) |
| for TspE4C2. | (SEQ ID NO: 119) |

These pairs of primers generate, by amplification, fragments of 279 bp, of 211 bp and of 152 bp, respectively.

According to a simplified protocol, a two-step triplex polymerase reaction was used. The components of the reaction are the same as in the standard protocol, except

(i) the DNA was provided directly by 3 μl of bacterial lysate or a colony fraction,

(ii) the six primers mentioned above were mixed together,

(iii) the PCR steps were as follows: denaturation for 4 minutes at 94° C., 30 cycles of 5 seconds at 94° C. and 10 seconds at 59° C., and a final extension step of 5 minutes at 72° C.

Southern transfer. The Southern transfer was carried out by transfer by capillarity onto positively charged nylon membranes. The hybridization was carried out at 65° C. in 1% SDS/1M NaCl/50 mM Tris HCl, pH 7.5/1% of blocking agent (Boehringer Mannheim, Mannheim, Germany). The membranes were washed in 2×SSC for 15 minutes at room temperature; then in 2×SSC/0.1% SDS for 30 minutes at 65° C. and finally, in 0.1×SSC for 5 minutes at room temperature. The detection of chemiluminescence was carried out according to the manufacturer's instructions (DIG Luminescence Detection Kit for nucleic acids, Boehringer Mannheim). The probes were produced by PCR according to the manufacturer's instructions (PCR DIG Probe Synthesis Kit, Boehringer Mannheim) and using the primers and the amplification procedure described above for the standard protocol.

Results

Two hundred and twenty strains were analysed. Their phylogenic groups determined by reference methods are as follows: 43 strains belong to group A, 23 to group B1, 41 to group D and 113 to group B2.

TABLE 6
PCR amplification of genes chuA et yjuA and of TSPE4.C2 DNA
fragment in E. coli strains of various
collections, depending on their phylogenic group.

| | | Groups according | Number of strains | | |
|---|---|---|---|---|---|
| Strains or isolates | | to RM (number of | having a positive amplification | | |
| collection | Method | strains) | chuA | yjuA | TSPE4.C2 |
| ECOR | MLEE | A(25) | 0 | 18(72%) | 0 |
| (n = 68) | | B1(16) | 0 | 1(6%) | 15(94%) |
| | | D(12) | 12(100%) | 0 | 2(17%) |
| | | B2(15) | 15(100%) | 15(100%) | 12(80%) |
| Neonatal meningitis | Ribotyping | A(5) | 0 | 5(100%) | 0 |
| ((n = 86) | | B1(3) | 0 | 1(33%) | 2(66%) |
| | | D(18) | 18(100%) | 0 | 2(11%) |
| | | B2(60) | 60(100%) | 60(100%) | 59(98%) |
| Other clinical | Method of the | A(12) | 0 | 9(75%) | 0 |
| strains (n = 64) | invention | B1(4) | 0 | 0 | 4(100%) |
| | | D(11) | 11(100%) | 0 | 1(9%) |
| | | B2(37) | 37(100%) | 37(100%) | 34(92%) |
| E. coli K12 | MLEE | A(1) | 0 | 1 | 0 |
| E. coli J96 | Ribotyping | B2(1) | 1 | 1 | 0 |
| | (this study) | | | | |
| Other clinical | | A(43) | 0 | 33(77%) | 0 |
| strains (n = 220) | | B1(23) | 0 | 2(9%) | 21(91%) |
| | | D(41) | 41(100%) | 0 | 5(12%) |
| | | B2(113) | 113(100%) | 113(100%) | 105(93%) |

R.M: phylogenic groups assessed by reference methods (MLEE or ribotyping)
MLEE: multilocus enzymatic electrophoresis Herzer et al. 1990, J. Bacteriol. 172: 6175-6178
Ribotyping: Binzen et al. 1998, J. Infect. Dis. 177: 642-650

Table 6 above shows the results obtained with the method according to the invention and the reference methods for the complete set of strains, according to phylogenic group. The chuA gene is present in all the strains belonging to groups B2 and D, and absent from all the strains of groups A and B1. This makes it possible to effectively separate groups B2/D from groups A/B1. In the same way, the yjaA gene allows complete distinction between group B2 (100% positive strains) and group D (100% negative strains). Finally, the novel TspE4.C2 clone is present in all the strains of group B1 except 2, and absent from all the strains of group A. All the PCR results were confirmed by Southern hybridization. The results of these three amplifications made it possible to establish a dichotomous decision tree for the phylogenic grouping. This decision tree is in particular illustrated in FIG. 3. Following this tree, 218 of the 220 strains tested (99%) are correctly grouped with respect to the reference methods, while only two strains which are considered to belong to group B1 according to the reference methods are identified as being from group A with the technique according to the invention. Identical results were obtained with the standard and simplified PCR protocols. FIG. 4 illustrates the various profiles which were obtained by triplex PCR for the four phylogenic groups. These novel profiles therefore constitute analytical references for the phylogenic grouping of E. coli.

Discussion

The inventors have developed a PCR method for rapidly determining the phylogenic group of E. coli strains. Using two genes, chuA and yjaA, and a novel DNA fragment named TspE4.C2 (SEQ ID NO: 119, cf. Example 1), the phylogenic groups of 220 strains which had previously been assigned to phylogenic groups determined using known methods were determined. The precision of analysis obtained according to the invention exceeds 99% with respect to the grouping established with the methods of the prior art. In addition, the same results were observed with a technically very simple triplex PCR method which is used directly on the bacterial colonies.

The phylogenic characterization of the E. coli strains, based on a few genotypic or phenotypic characteristics, appeared, in the prior art, to be very difficult. Such genotypic characteristics (presence or absence of a gene, for example) must satisfy various criteria in order to be able to be used for phylogenic characterization. First of all, the gene must have been acquired, or have been deleted, when the group that it characterizes emerged. Secondly, this same gene must have been “stabilized” so as to exclude any phenomenon of subsequent deletion or horizontal transfer towards bacteria belonging to other phylogenic groups. Finally, recombination phenomena must be very rare in the candidate gene. In other words, the gene product must not be a target for natural selection, which would favour novel genetic recombinations. In the prior art, the attempts to identify characteristics of phylogenic groups based on the phenotype or on the genotype have not been shown to be sufficiently discriminating. For the first time, described herein, is the combined use of two genes and of a novel DNA fragment which make it possible, in a technically very simple way, to determine the phylogenic group with good effectiveness.

However, two strains (ECOR 70 and an NMEC) belonging, according to conventional techniques, to the phylogenic group B1 were classified, by the method according to the invention, in group A. This analytical difference may be explained by the fact that there may exist an intermediate genetic base common to these two groups of strains, and by the fact that the regions studied using the method according to the invention (chuA, yjaA and TspE4.C2, are located at 78.7 minutes, 90.8 minutes and approximately at 87 minutes, respectively, on the genome of E. coli K12) might be closer to the group A than the regions studied using the methods of the prior art. Specifically, it has been demonstrated that the groups A and B1 are sister groups. In addition, recent analyses of multiple chromosomal nucleotide sequences show that ECOR 70 can be considered to be a “hybrid” strain in which a few housekeeping genes have nucleotide sequences which are common with ECOR strains of group A, and in which few other genes have nucleotide sequences which are common with the ECOR strains of group B1. The phylogenic membership of ECOR 70 to group B1 is not clearly determined; it is, in any event, considered to be part of group A using the method according to the invention.

In addition to the rapidity of our novel PCR method, the invention has the advantage of not requiring the use of a reference collection such as the ECOR strain or another collection, which means that the analysis can be easily carried out in the laboratory and in particular for routine analyses. In addition, unlike the other methods, the allocation to the phylogenic groups is unequivocal. Specifically, the 4 strains which, until now, were unclassified in the ECOR collection (E31, E37, E42 and E43) can be classified using the method of the invention; the first three strains belong to group D, and the fourth to group A. It can also be noted that all the sequences of the housekeeping genes studied in the latter strain appear to be characteristic of those found in the strains of group A.

In conclusion, this simple and rapid phylogenic grouping technique has many practical uses. The first use is, of course, the bioclinical use, taking into account the link which can be established between the phylogenic group and the possible virulence or dangerousness. The second use corresponds to a biotechnological screening tool which makes it possible to eliminate potentially pathogenic strains, i.e. strains which are highly dangerous, when candidate strains are being sought for cloning. It was possible to develop such screening tools in the prior art, for example for identifying the E. coli K12 strains by PCR or for detecting E. coli strains which exhibit none of the virulence genes which were, until then, known due to a reverse dot blot procedure. The method described herein has the notable advantage of allowing the identification of nonpathogenic strains other than E. coli K12, and of being suitable for screening strains on a large scale.

### EXAMPLE 6

Production of Seventy CFT073+ K12− Zones and Thirty One RS218+/K12− Zones

In the previous examples, the production of a library of clones, which are of nature B2/D+ A−, by substrative hybridization between the E. coli strain C5 (E. coli strain belonging to group B2) and E. coli strains of group A (nonpathogenic E. coli strains ECOR4 and ECOR15), is described. The sequence of these clones is given in FIG. 2 (SEQ ID NO: 1-153 and 169-253).

These clones are present with greater frequency in the ECOR E. coli of group B2 and/or in the ECOR E. coli of group D with respect to the ECOR E. coli of group A (preferably 2 times greater, more preferably 3 times greater, even more preferably 3.5 times greater, and very preferably 4 times greater). They are, in particular, present at a frequency greater than 10% in the ECOR E. coli of group B2 and/or in the ECOR E. coli of group D, and at a frequency of less than 25%, preferably less than 20%, more preferably less than 10%, and even more preferably less than 5% in the ECOR E. coli of group A, the frequency observed in the ECOR E. coli of group A always remaining less than that observed in the ECOR E. coli of group B2 and/or in the ECOR E. coli of group D.

The presence of these clones could, moreover, be verified in various E. coli strains of group B2 or D which are involved in pathologies which are entirely different from that in which the E. coli strain C5, which was initially used for isolating these clones, participates. The presence of these clones was, for example, verified in E. coli CFT073 (E. coli of group B2 involved in adult pyelonephritis).

In doing so, it was observed that some of these clones lie, at the level of the chromosome of E. coli CFT073, within polynucleotide zones which are not found in E. coli K12 MG1655. CFT073+ K12− zones were thus isolated in the vicinity of said fragments which are of nature B2/C+ A− (isolated from E. coli C5; cf. Example 1). An illustration is given herein for seventy of them.

The chromosomal position of each of these seventy CFT073+ K12− zones was then determined (“K12 coordinates” column in Table 8 below) with respect to the chromosomal map of E. coli K12, which constitutes a reference in the field. These seventy CFT073+ K12− zones were then analysed in order to determine the possible presence of open reading frames (orf/ORF) using programs such as CodonUse™ (E. coli codon use) and ORF Finder (www.ncbi.nlm.nih.gov/gorf/gorf.html, bacterial genetic code). The corresponding protein sequences (ORFs) were extracted compared to various databases in order to search for homology compared to known sequences. The sequences of these 70 zones, and of their ORFs, are represented in FIG. 6. For each zone, the protein sequence (ORF) and polynucleotide sequence (orf) of the open reading frame identified is shown, indicating the start and end positions (Pos.) of these frames with respect to the complete sequence of the zone concerned. Some of these ORFs are encoded by orfs which are, in fact, located on the strand complementary to the indicated sequence of the region (the “frame start” position then has a number which is higher than that of the “frame end” position).

The same protocol was applied to RS218 E. coli clones. RS218+/K12− zones have also been isolated in the neiboughring of the fragments with a B2/D+ A− nature.

Table 7 below summarizes the SEQ ID NOS assigned to each of the seventy CFT073+ K12− zones and to their ORFs and orfs, and each thirty one RS218+/K12− zones.

TABLE 7

| Zone | SEQ ID NO | Reading frames |
|---|---|---|
| number | of the Zone | (SEQ ID No of the ORF; SEQ ID NO of the Orf |
| 1 | 256 | (258; 257) (260; 259) |
| 2 | 261 | (263; 262) (265; 264) (267; 266) |
| | | (269; 268) (271; 270) (272; 273) |
| 3 | 274 | (276; 275) (278; 277) (280; 279) (282; 281) |
| 4 | 283 | (285; 284) (287; 286) |
| 5 | 288 | (290; 289) (292; 291) |
| | | (294; 293) (296; 295) |
| 6 | 297 | (299; 298) (301; 300) (303; 302) |
| | | (305; 304) (307; 306) (309; 308) (311; 310) |
| 7 | 312 | (314; 313) |
| 8 | 315 | (317; 316) (319; 318) |
| | | (321; 320) (323; 322) (325; 324) |
| 9 | 326 | (328; 327) (329; 330) (332; 331) |
| | | (334; 333) (336; 335) (338; 337) |
| | | (340; 339) (342; 341) |
| 10 | 343 | (345; 344) |
| 11 | 346 | (348; 347) (350; 349) (352; 351) |
| 12 | 353 | (355; 354) (357; 356) (359; 358) (361; 360) |
| 13 | 362 | (364; 363) (366; 365) (368; 367) (370; 369) |
| | | (372; 371) (374; 373) (376; 375) (378; 377) |
| 14 | 379 | (381; 380) (383; 382) (385; 384) |
| | | (387; 386) (389; 388) (391; 390) |
| | | (393; 392) (395; 394) (397; 396) (399; 398) |
| 15 | 400 | (402; 401) |
| 16 | 403 | (405; 404) (407; 406) |
| 17 | 408 | (410; 409) (412; 411) (414; 413) (416; 415) |
| 18 | 417 | (419; 418) |
| 19 | 420 | (426; 425) (428; 427) (430; 429) |
| 20 | 431 | (433; 432) (435; 434) (437; 436) |
| | | (439; 438) (441; 440) (443; 442) |
| 21 | 444 | (446; 445) (448; 447) |
| 22 | 449 | (451; 450) (453; 452) (455; 454) |
| 23 | 456 | (458; 457) (460; 459) |
| 24 | 461 | (463; 462) (465; 464) (467; 466) (469; 468) |
| | | (471; 470) (473; 472) (475; 474) |
| 25 | 476 | (478; 477) (480; 479) (482; 481) |
| | | (484; 483) (486; 485) |
| 26 | 487 | (489; 488) |
| 27 | 490 | (492; 491) (494; 493) (496; 495) (498; 497) |
| | | (500; 499) (502; 501) (504; 503) (506; 505) |
| 28 | 507 | (509; 508) (511; 510) (513; 512) |
| | | (515; 514) (517; 516) (519; 518) |
| | | (521; 520) (523; 522) (525; 524) |
| | | (527; 526) (529; 528) (531; 530) (533; 532) |
| 29 | 534 | (536; 535) (538; 537) (540; 539) |
| | | (542; 541) (544; 543) (546; 545) |
| 30 | 547 | (549; 548) (551; 550) (553; 552) |
| 31 | 554 | (556; 555) (558; 557) (560; 559) (562; 561) |
| | | (564; 563) (566; 565) (568; 567) |
| 32 | 569 | (571; 570) (573; 572) (575; 574) (577; 576) |
| 33 | 578 | (580; 579) (582; 581) |
| 34 | 583 | (585; 584) (587; 586) (589; 588) |
| 35 | 590 | (592; 591) (594; 593) (596; 595) |
| 36 | 597 | (599; 598) (601; 600) (603; 602) |
| | | (605; 604) (607; 606) (609; 608) |
| 37 | 610 | (612; 611) (614; 613) (616; 615) (618; 617) |
| | | (620; 619) (622; 621) (624; 623) (626; 625) |
| | | (628; 627) (630; 629) (632; 631) (634; 633) |
| 38 | 635 | (637; 636) (639; 638) (641; 640) |
| | | (643; 642) (645; 644) (647; 646) |
| 39 | 648 | (650; 649) (652; 651) |
| 40 | 653 | (655; 654) (657; 656) (659; 658) (661; 660) |
| 41 | 662 | (664; 663) |
| 42 | 665 | (667; 666) (669; 668) (671; 670) |
| | | (673; 672) (675; 674) |
| 43 | 676 | (678; 677) (680; 679) (682; 681) |
| | | (684; 683) (686; 685) |
| 44 | 687 | (689; 688) (691; 690) (693; 692) |
| | | (695; 694) (697; 696) (699; 698) |
| 45 | 700 | (702; 701) (704; 703) |
| 46 | 705 | (707; 706) (709; 708) (711; 710) |
| | | (713; 712) (715; 714) |
| 47 | 716 | (718; 717) |
| 48 | 719 | (721; 720) (723; 722) (725; 724) (727; 726) |
| 49 | 728 | (730; 729) (732; 731) (734; 733) |
| | | (736; 735) (738; 737) (740-739) |
| | | (742; 741) (744; 743) |
| 50 | 745 | (747; 746) (749; 748) |
| 51 | 750 | (752; 751) (754; 753) (756; 755) |
| | | (758; 757) (760; 759) (762; 761) |
| | | (764; 763) |
| 52 | 765 | (767; 766) (769; 768) (771; 770) |
| | | (773; 772) |
| 53 | 774 | (776; 775) (778; 777) (780; 779) |
| | | (782; 781) (784; 783) (786; 785) |
| | | (788; 787) (790; 789) (792; 791) |
| | | (794; 793) (796; 795) (798; 797) |
| | | (800; 799) (802; 801) (804; 803) |
| | | (806; 805) (808; 807) (810; 809) |
| | | (812; 811) (814; 813) |
| 54 | 815 | (817; 816) (819; 818) (821; 820) |
| 55 | 822 | (824; 823) (826; 825) (828; 827) |
| 56 | 829 | (831; 830) (833; 832) (835; 834) (837; 836) |
| | | (839; 838) (841; 840) (843; 842) (845; 844) |
| | | (847; 846) (849; 848) (851; 850) (853; 852) |
| | | (855; 854) (857; 856) (859; 858) (861; 860) |
| | | (863; 862) (865; 864) (867; 866) (869; 868) |
| | | (871; 870) (873; 872) (875; 874) (877; 876) |
| | | (879; 878) (881; 880) (883; 882) (885; 884) |
| | | (887; 886) (889; 888) (891; 890) (893; 892) |
| | | (895; 894) (897; 896) (899; 898) (901; 900) |
| | | (903; 902) (905; 904) (907; 906) (909; 908) |
| | | (911; 910) (913; 912) (915; 914) (917; 916) |
| | | (917.1; 916.1) |
| 57 | 920 | |
| 58 | 921 | (923; 922) (925; 924) (927; 926) (929; 928) |
| | | (931; 930) (933; 932) (935; 934) (937; 936) |
| | | (939; 938) (941; 940) (943; 942) (945; 944) |
| | | (947; 946) (949; 948) (951; 950) (953; 952) |
| | | (955; 954) (957; 956) (959; 958) (961; 960) |
| | | (963; 962) (965; 664) (967; 966) (969; 968) |
| | | (971; 970) |
| 59 | 972 | (974; 973) (976; 975) (978; 977) (980; 979) |
| | | (982; 981) (984; 983) (986; 985) (988; 987) |
| | | (990; 989) (992; 991) |
| 60 | 993 | (995; 994) (997; 996) (999; 998) |
| | | (1001; 1000) (1003; 1002) (1005; 1004) |
| | | (1007; 1006) (1009; 1008) (1011; 1010) |
| | | (1013; 1012) (1015; 1014) (1017; 1016) |
| | | (1019; 1018) (1021; 1020) (1023; 1022) |
| | | (1025; 1024) (1027; 1026) (1029; 1028) |
| | | (1031; 1030) (1033; 1032) (1035; 1034) |
| | | (1037; 1036) |
| 61 | 1038 | (1040; 1039) (1042; 1041) (1044; 1043) |
| | | (1046; 1045) (1048; 1047) |
| 62 | 1049 | (1051; 1050) (1053; 1052) (1055; 1054) |
| | | (1057; 1056) (1059; 1058) (1061; 1060) |
| | | (1063; 1062) (1065; 1064) (1067; 1066) |
| | | (1069; 1068) (1071; 1070) (1073; 1072) |
| | | (1075; 1074) (1077; 1076) |
| 63 | 1078 | (1080; 1079) (1082; 1081) |
| 64 | 1083 | (1085; 1084) (1087; 1086) (1089; 1088) |
| | | (1091; 1090) (1093; 1092) (1095; 1094) |
| 65 | 1096 | (1098; 1097) (1100; 1099) (1102; 1101) |
| | | (1104; 1103) (1106; 1105) (1108; 1007) |
| | | (1110; 1109) (1112; 1111) (1114; 1113) |
| | | (1116; 1115) |
| 66 | 1117 | (1119; 1118) (1121; 1120) (1123; 1122) |
| | | (1125; 1124) (1127; 1126) (1129; 1128) |
| | | (1131; 1130) (1133; 1132) (1135; 1134) |
| | | (1137; 1136) (1139; 1138) (1141; 1140) |
| | | (1143; 1142) (1145; 1144) (1147; 1146) |
| | | (1149; 1148) (1151; 1150) (1153; 1152) |
| | | (1155; 1154) (1157; 1156) (1159; 1158) |
| | | (1161; 1160) (1163; 1162) (1165; 1164) |
| | | (1167; 1166) (1169; 1168) (1171; 1170) |
| | | (1173; 1172) (1175; 1174) (1177; 1176) |
| | | (1179; 1178) (1181; 1180) (1183; 1182) |
| | | (1185; 1184) |
| 67 | 1186 | (1188; 1187) (1190; 1189) |
| 68 | 1191 | (1193; 1192) (1195; 1194) (1197; 1196) |
| | | (1199; 1198) (1201; 1200) (1203; 1202) |
| | | (1205; 1204) (1207; 1206) (1209; 1208) |
| | | (1211; 1210) (1213; 1212) (1215; 1214) |
| | | (1215.1; 1214.1) (1215.2; 1214.2) |
| 69 | 1216 | (1218; 1217) (1220; 1219) (1222; 1221) |
| | | (1224; 1223) (1226; 1225) (1228; 1227) |
| | | (1230; 1229) (1232; 1231) (1234; 1233) |
| | | (1236; 1235) (1238; 1237) (1240; 1239) |
| | | (1242; 1241) (1244; 1243) (1244.1; 1243.1) |
| 70 | 1245 | (1247; 1246) (1249; 1248) |
| 71 | 1250 | (1252; 1251) (1254; 1253) (1256; 1255) |
| | | (1258; 1257) (1260; 1259) (1262; 1261) |
| | | (1264; 1263) (1266; 1265) (1268; 1267) |
| | | (1270; 1269) (1272; 1271) |
| 72 | 1273 | (1275; 1274) (1277; 1276) (1279; 1278) |
| 73 | 1280 | (1282; 1281) (1284; 1283) (1286; 1285) |
| | | (1288; 1287) |
| 74 | 1289 | (1291; 1290) (1293; 1292) (1295; 1294) |
| 75 | 1296 | (1298; 1297) |
| 76 | 1299 | (1301; 1300) (1303; 1302) |
| 77 | 1304 | (1306; 1305) |
| 78 | 1307 | (1309; 1308) (1311; 1310) (1313; 1312) |
| | | (1315; 1314) (1317; 1316) |
| 79 | 1318 | (1320; 1319) (1322; 1321) (1324; 1323) |
| | | (1326; 1325) (1328; 1327) (1330; 1329) |
| 80 | 1331 | (1333; 1332) (1335; 1334) (1337; 1336) |
| | | (1339; 1338) (1341; 1340) (1343; 1342) |
| | | (1345; 1344) (1347; 1346) (1349; 1348) |
| | | (1351; 1350) (1353; 1352) |
| 81 | 1354 | (1356; 1355) |
| 82 | 1357 | |
| 83 | 1358 | (1360; 1359) (1362; 1361) (1364; 1363) |
| | | (1366; 1365) (1368; 1367) (1370; 1369) |
| | | (1372; 1371) |
| 84 | 1373 | (1375; 1374) (1377; 1376) (1379; 1378) |
| | | (1381; 1380) (1383; 1382) |
| 85 | 1386 | (1388; 1387) (1390; 1389) (1392; 1391) |
| | | (1394; 1393) (1396; 1395) (1398; 1397) |
| 86 | 1399 | (1401; 1400) (1403; 1402) (1405; 1404) |
| | | (1407; 1406) (1409; 1408) |
| 87 | 1410 | (1412; 1411) (1414; 1413) (1416; 1415) |
| | | (1418; 1417) (1420; 1419) (1422; 1421) |
| | | (1424; 1423) |
| 88 | 1425 | (1427; 1426) (1429; 1428) (1431; 1430) |
| | | (1433; 1432) (1435; 1434) (1437; 1436) |
| | | (1439; 1438) (1441; 1440) (1443; 1442) |
| | | (1445; 1444) (1447; 1446) (1449; 1448) |
| | | (1451; 1450) (1453; 1452) |
| 89 | 1454 | (1456; 1455) (1458; 1457) (1460; 1459) |
| | | (1162; 1461) (1464; 1463) (1466; 1465) |
| 90 | 1467 | (1469; 1468) |
| 91 | 1470 | (1472; 1471) (1474; 1473) |
| 92 | 1475 | (1477; 1476) (1479; 1478) (1481; 1480) |
| 93 | 1482 | (1484; 1483) (1486; 1485) (1488; 1487) |
| | | (1490; 1489) (1492; 1491) (1494; 1493) |
| | | (1496; 1495) |
| 94 | 1497 | (1499; 1498) (1501; 1500) (1503; 1502) |
| 95 | 1504 | (1506; 1505) (1508; 1507) (1510; 1509) |
| | | (1512; 1511) (1514; 1513) (1516; 1515) |
| 96 | 1517 | (1519; 1518) (1521; 1520) (1523; 1522) |
| 97 | 1524 | |
| 98 | 1525 | (1527; 1526) (1529; 1528) (1531; 1530) |
| | | (1533; 1532) |
| 99 | 1534 | (1536; 1535) (1538; 1537) (1540; 1539) |
| 100 | 1541 | (1543; 1542) (1545; 1544) (1547; 1546) |
| | | (1549; 1548) (1551; 1550) (1553; 1552) |
| | | (1555; 1554) (1557; 1556) (1559; 1558) |
| | | (1561; 1560) |
| 101 | 1562 | (1564; 1563) (1566; 1565) (1568; 1567) |

Tables 8 and 9 below summarizes the results obtained on SEQ IDs; the following are indicated:

Table 8:—the SEQ ID NOs and the names of the clones having allowed the identification of each CFT073+ K12− zone and RS218+/K12− zone, the number which was assigned to this zone, the coordinates of each zone with respect to the chromosomal map of E. coli K12, the respective size of each zone, the number of the fragment, the number of the region within which the said zone is located (regions 1, 2, 3, 4, 5, 6a or 6b as identified in Examples 1 and 3 above; or, if the zone is not within a region, indicated in this column is the name of the E. coli RS218 fragment at the level of which this zone is located, cf. FIG. 1 for the E. coli RS218 fragment names),

Table 9:—the homology with E. coli RS218, E. coli 0157:H7, E. coli CFT073.

TABLE 8

| | | Coordinate on the | | | position of | SEQ ID NO | Overlap | |
|---|---|---|---|---|---|---|---|---|
| | | E.coli K12 | | | the clone | of the (ORF, orf) | with an | Overlap |
| | clone(s) on | chromosome | Size of the | | on the | encompassing | intergenic | between |
| fragment | fragment | of the fragment | fragment | Region | fragment | the clone | sequence | two orfs |
| 1 | SauE15.C12 | 3170205-3170387 | 1312 | | | | | |
| 2 | SauE15.C12 | 3171462-3171524 | 7316 | | 5993-6148 | (263; 262) | | |
| 3 | SauE15.M11 | 2163577-2163599 | 5203 | | 1800-1966 | (278; 277) | | |
| 4 | SauE4.H6 | 4264441-4264615 | 1584 | | 1343-1585 | (285; 284) | + | |
| 5 | TspE4.C2 | 4076018-4076534 | 2444 | 5 | 743-945 | (290; 289) | | |
| 5 | TspE15.G3 | 4076018-4076534 | 2444 | | 2094-2419 | (296; 295) | + | |
| 6 | TspE4.C3 | 4061139-4073066 | 8644 | 5 | 5376-5559 | (307; 306) | | |
| 7 | TspE4.H3 | 4085618-4090704 | 1835 | | 154-333 | — | | |
| 8 | SauE15.F12 | 4129846-4130052 | 7648 | 5 | 1514-1737 | (319; 318) | | |
| 8 | TspE15.A10 | 4129846-4130052 | 7648 | | 1623-1874 | (319; 318) | | |
| 8 | SauE4.G9 | 4129846-4130052 | 7648 | | 6596-6775 | (325; 324) | | |
| 8 | SauE4.F9 | 4129846-4130052 | 7648 | | 4029-4467 | (321; 320) | + | |
| 8 | TspE4.A7 | 4129846-4130052 | 7648 | 5 | 1377-1618 | (319; 318) | | |
| 9 | TspE15.H3 | 705240-705186 | 9137 | | 4170-4432 | (334; 333) | | |
| 10 | SauE15.A5 | 1388748-1388748 | 665 | | 379-570 | (345; 344) | + | |
| 11 | SauE15.E9 | 17126-17342 | 4144 | | 1203-1316 | (350; 349) | | |
| 11 | SauE15.E12 | 17126-17342 | 4144 | | 1483-1587 | (350; 349) | | |
| 11 | TspE15.E10 | 17126-17342 | 4144 | | 335-574 | (348; 347) | + | |
| 12 | TspE4.E7 | 4590882-4592607 | 5426 | 6a | 2416-2616 | (359; 358) | | |
| 13 | TspE4.K4 | 3404903-3405013 | 8681 | | 8402-8592 | (378; 377) | + | |
| 13 | SauE4.H7 | 3404903-3405013 | 8681 | | 7506-7869 | (376; 375) | + | |
| 14 | SauE4.A11 | 3561151-3561762 | 9973 | | 1741-1801 | (385; 384) | | |
| 14 | TspE15.H9 | 3561151-3561762 | 9973 | | 9109-9381 | (399; 398) | | |
| 15 | SauE15.L11 | 3754245-3754254 | 1370 | a (Notl) | 346-661 | (402; 401) | | |
| 16 | TspE4.D1 | 2753977-1432792 | 1692 | | 1097-1333 | (407; 406) | + | |
| 17 | SauE4.B1 | 1430899-1427392 | 3761 | | 523-676 | (410 ; 409) | | |
| 17 | TspE15.G6 | 1430899-1427392 | 3761 | | 1375-1655 | (414 ; 413) (412 ; 411) | | + |
| 18 | SauE4.B1 | 1427076-1427064 | 726 | | | | | |
| 19 | TspE15.G7 | 2471606-2471605 | 3270 | | 2539-2823 | (430 ; 429) | | |
| 20 | TspE4.K1 | 2473509-2786860 | 4585 | | 1728-1905 | (435; 434) | | |
| 20 | TspE15.E5 | 2473509-2786860 | 4585 | | 1910-2140 | (435; 434) | | |
| 20 | SauE15.A4 | 2473509-2786860 | 4585 | | 4418-4586 | (443; 442) | | |
| 21 | SauE15.L8 | 2798629-2798551 | 1951 | 3 | 1164-1366 | (448; 447) | | |
| 22 | TspE15.G5 | 2903529-2903946 | 2981 | | 2040-2447 | (455; 454) | | |
| 23 | TspE4.E6 | 4225080-4225293 | 1036 | | | | | |
| 24 | TspE4.E6 | 4227811-4227898 | 6318 | | 685-874 | (463; 462) | | |
| 24 | SauE4.H2 | 4227811-4227898 | 6318 | | 2282-2366 | (465; 464) | + | |
| 24 | SauE4.F8 | 4227811-4227898 | 6318 | | 5868-6055 | (475; 474) | | |
| 24 | SauE15.I12 | 4227811-4227898 | 6318 | | 6060-6266 | (475; 474) | + | |
| 25 | SauE4.H6 | 4261810-4261877 | 7325 | | | | | |
| 26 | SauE4.H6 | 4263309-4263352 | 712 | | | | | |
| 27 | SauE4.H6 | 4265884-4266886 | 13757 | | | | | |
| 27 | SauE15.N9 | 4265884-4266886 | 13757 | | 5819-5927 | (496; 495) | | |
| 27 | TspE4.B11 | 4265884-4266886 | 13757 | | 5985-6175 | (496; 495) | | |
| 28 | SauE15.I7 | 2068681-121950 | 12263 | | 2998-3357 | (517; 516)(519; 518) | | + |
| 28 | SauE15.J2 | 2068681-121950 | 12263 | 6b | 5595-6316 | (523; 522) | + | |
| 28 | TspE4.K12 | 2068681-121950 | 12263 | | 6214-6392 | (523; 522) | | |
| 28 | TspE4.D8 | 2068681-121950 | 12263 | 2 | 6673-6819 | (523; 522) | | |
| 28 | TspE15.D9 | 2068681-121950 | 12263 | | 8608-8836 | (527; 526) | | |
| 28 | SauE15.F3 | 2068681-121950 | 12263 | | 9834-10254 | (527; 526) | | |
| 29 | TspE4.K10 | 237013-207669 | 4871 | 6b | 4045-4295 | (546; 545) | | |
| 29 | SauE4.E10 | 237013-207669 | 4871 | | 4149-4346 | (546; 545) | | |
| 29 | SauE15.A6 | 237013-207669 | 4871 | | 2104-2232 | (544; 543) | + | |
| 30 | TspE4.G11 | 764371-770373 | 2777 | f (notl) | 824-1018 | (549; 548) | | |
| 30 | TspE4.A3 | 764371-770373 | 2777 | f (Notl) | 1603-1815 | (553; 552) | | |
| 31 | SauE15.N6 | 262172-302054 | 7655 | 1 | 4969-5163 | (560; 559) | | |
| 31 | SauE15.H11 | 262172-302054 | 7655 | | 4805-4964 | (560; 559) | | |
| 32 | TspE4.F4 | 324698-324697 | 6000 | | 3774-3998 | (575; 574) | | |
| 33 | SauE15.A11 | 1588333-1588774 | 2497 | c (Notl) | | | | |
| 34 | SauE15.I3 | 3179593-3179639 | 3707 | | 1287-1462 | (585; 584) | | |
| 35 | SauE15.D11 | 3183632-3181831 | 4821 | a (Notl) | 3972-4214 | (596; 595) | | |
| 36 | TspE4.K9 | 3829794-3829838 | 7267 | | 7066-7268 | — | | |
| 36 | TspE15.F9 | 3829794-3829838 | 7267 | | 6340-6583 | (609; 608) | | |
| 37 | SauE15.A12 | 4012900-4012961 | 16066 | | 494-667 | — | | |
| 37 | SauE4.G11 | 4012900-4012961 | 16066 | | 672-921 | (634; 633) | | |
| 37 | TspE15.A3 | 4012900-4012961 | 16066 | | 9678-9884 | (622; 621) | + | |
| 37 | SauE15.M10 | 4012900-4012961 | 16066 | 5 | 14902-15198 | (630; 629)(632; 631) | | + |
| 38 | TspE15.D3 | 4014776-4014824 | 5601 | | 643-845 | (637; 636) | | |
| 39 | SauE4.C7 | 4158487-4158562 | 2703 | | 888-1162 | (650; 649) | | |
| 40 | TspE15.E1 | 2633462-2633903 | 12101 | | 3556-4004 | (655; 654) | | |
| 41 | SauE4.B9 | 3077427-3077662 | 1423 | | 459-638 | (664; 663) | | |
| 42 | SauE15.N4 | 3108458-4497174 | 3129 | | | | | |
| 43 | TspE4.D12 | 4294686-4294793 | 5571 | | 4687-4908 | (686; 685) | | |
| 44 | TspE4.B4 | 1093420-3290368 | 8739 | l (Notl) | 2652-2869 | (691; 690) | | |
| 45 | SauE15.E7 | 3290119-3281094 | 3245 | | 1808-1967 | (702; 701) | + | |
| 45 | TspE15.G12 | 3290119-3281094 | 3245 | | 2039-2325 | (704; 703) | | |
| 46 | SauE4.D9 | 4470142-4474549 | 6164 | | 17-286 | (707; 706) | | |
| 46 | TspE4.H1 | 4470142-4474549 | 6164 | | 4930-5212 | (715; 714) | | |
| 47 | SauE15.H2 | 4477050-4478549 | 1478 | | 1104-1204 | (718; 717) | | |
| 62 | TspE4.D3 | 2074323-? | 16244 | 2 | 1463-1536 | (1055; 1054) | + | |
| 62 | TspE15.H11 | 2074323-? | | | 8239-8348 | — | | |
| 62 | SauE4.H10 | 2074323-? | | | 8239-8349 | — | | |
| 62 | SauE15.N1 | 2074323-? | | 6a | 3498-3700 | (1061; 1060) | | |
| 63 | SauE4.E6 | — | | | 229-417 | (1080; 1079) | | |
| 64 | SauE15.H7 | ?-313031 | 6202 | | 5380-5529 | (1095; 1094) | | |
| 65 | TspE15.C3 | 1529850-1531310 | 12133 | | 6264-6334 | (1108; 1107) | | |
| 66 | TspE15.H7 | — | | | 1155-1374 | — | | |
| 66 | TspE4.A10 | — | | | 32724-32935 | (1121; 1120) | | |
| 67 | SauE4.B8 | — | | | 1343-1374 | (1190; 1189) | | |
| 68 | TspE15.H7 | ?-2074324 | | | 1108-1327 | — | | |
| 68 | TspE4.D3 | ?-2074324 | | 2 | 15238-15364 | — | | |
| 69 | SauE15.K5 | — | | 6a | 1043-1267 | — | | |
| 69 | SauE15.L6 | — | | 6a | 757-1038 | (1230; 1229) | + | |
| 69 | SauE4.E5 | — | | | 546-749 | (1230; 1229) | | |
| 69 | TspE4.K2 | — | | | 1540-1744 | (1232; 1231) | + | |
| 70 | SauE15.J11 | — | 1350 | | 741-974 | (1247; 1246)(1249; 1248) | | + |
| 70 | SauE15.K10 | — | 1350 | | 982-1106 | (1249; 1248) | | |
| 71 | SauE4.C10 | — | 11475 pb | | 8272-8511 | (1268; 1267) | + | |
| 71 | SauE15.B10 | — | 11475 pb | | 4117-4356 | (1260; 1259)(1262; 1261) | | + |
| 71 | SauE4.A2 | — | 11475 pb | | 5636-5955 | (1264; 1263) | + | |
| 72 | SauE15.C7 | — | 4576 pb | | 3445-3599 | (1277; 1276) | | |
| 72 | SauE15.M12 | — | 4576 pb | | 1334-1452 | (1275; 1274) | | |
| 72 | TspE4.B9 | — | 4576 pb | | 520-838 | (1275; 1274) | | |
| 72 | SauE15.E4 | — | 4576 pb | | 425-705 | (1275; 1274) | | |
| 73 | SauE15.H3 | — | 1906 pb | 6a | 1696-1898 | (1288; 1287) | + | |
| 74 | SauE15.A9 | — | 2536 pb | Not d | 500-624 | (1291; 1290)(1293; 1292) | | + |
| 75 | TspE4.H5 | — | 894 pb | 1 | 466-773 | (1298; 1297) | | |
| 76 | TspE15.A2 | 2137782-2137508 | 747 pb | | 129-336 | (1301; 1300) | | |
| 77 | TspE15.H10 | — | 886 pb | | 119-413 | (1306; 1305) | | |
| 78 | TspE15.H2 | — | 4892 pb | | 1990-2247 | (1315; 1314) | | |
| 79 | TspE15.I4 | 2099320-? | 6927 pb | | 4446-4688 | (1328; 1327) | | |
| 80 | TspE4.A4 | 4261856-4262018 | 10848 pb | | 9568-9976 | (1353; 1352) | | |
| 80 | SauE15.H8B | 4261856-4262018 | 10848 pb | | 9509-9668 | (1353; 1352) | | |
| 81 | TspE4.I7 | — | 1716 pb | | 1597-1716 | — | | |
| 82 | SauE4.E7 | 1633100-1633288 | 200 pb | | 43-168 | — | | |
| 83 | TspE4.H6 | — | 5873 pb | 3 | 5764-5873 | (1360; 1359) | | |
| 83 | SauE15.M9 | — | 5873 pb | 3 | 5329-5599 | (1360; 1359) | | |
| 83 | TspE15.D1 | — | 5873 pb | | 2330-2714 | (1366; 1365)(1368; 1367) | | + |
| 84 | SauE15.L9 | 3597559-? | 4737 pb | | 3752-3884 | (1383; 1382) | | |
| 84 | SauE15.L4 | 3597559-? | 4737 pb | 4 | 3562-3747 | (1383; 1382) | | |
| 84 | SauE15.K12 | 3597559-? | 4737 pb | 4 | 3337-3532 | (1381; 1380)(1383; 1382) | | + |
| 85 | TspE4.F12 | 4533846-2076361 | 4386 pb | 6a | 2733-2983 | (1390; 1389) | | |
| 85 | SauE4.C12 | 4533846-2076361 | 4386 pb | | 671-1060 | (1392; 1391) | + | |
| 86 | TspE4.G5 | 1529721-1524886 | 5819 pb | 1 | 449-645 | (1407; 1406) | | |
| 86 | TspE4.A12 | 1529721-1524886 | 5819 pb | 1 | 2489-2731 | (1403; 1402) | | |
| 86 | TspE15.F4 | 1529721-1524886 | 5819 pb | | 2081-2416 | (1403; 1402) | | |
| 87 | SauE15.I2 | — | 6066 pb | 1 | 5755-5936 | (1412; 1411) | | |
| 87 | TspE4.A1 | — | 6066 pb | 1 | 482-711 | (1422; 1421) | | |
| 87 | TspE15.F7 | — | 6066 pb | | 5100-5375 | (1414; 1413) | | |
| 88 | SauE15.I4 | 267058-381653 | 7890 pb | 6a | 6810-7006 | (1449; 1448) | + | |
| 88 | SauE15.N1 | 267058-381653 | 7890 pb | 6a | 2234-2436 | (1435; 1434) | | |
| 88 | TspE15.H11 | 267058-381653 | 7890 pb | | 4811-5050 | (1441; 1440) | + | |
| 88 | SauE4.H10 | 267058-381653 | 7890 pb | | 4849-5186 | (1441; 1440) | + | |
| 88 | SauE15.L7 | 267058-381653 | 7890 pb | | 7011-7150 | (1449; 1448) | | |
| 89 | SauE15.E8 | 2776123-1209029 | 5662 pb | | 756-1137 | (1458; 1457) | | |
| 89 | SauE15.N8 | 2776123-1209029 | 5662 pb | 3 | 345-603 | (1456; 1455)(1458 ; 1457) | | + |
| 89 | TspE15.D11 | 2776123-1209029 | 5662 pb | | 1592-1806 | (1460; 1459) | + | |
| 89 | TspE4.J12 | 2776123-1209029 | 5662 pb | | 4721-4910 | (1466; 1465) | | |
| 89 | TspE4.D1 | 2776123-1209029 | 5662 pb | 3 | 2904-3140 | (1464; 1463) | + | |
| 89 | TspE4.J10 | 2776123-1209029 | 5662 pb | | 5280-5524 | (1466; 1465) | | |
| 89 | SauE15.J12 | 2776123-1209029 | 5662 pb | | 3988-4091 | (1466; 1465) | | |
| 90 | TspE15.A1 | ?-4554943 | 1314 | | 1-273 | (1469; 1468) | + | |
| 90 | SauE15.I8 | ?-4554943 | 1314 | 6a | 713-1000 | (1469; 1468) | | |
| 91 | TspE4.J4 | — | 1581 pb | 1 | 274-505 | (1472:1471)(1474; 1473) | | + |
| 92 | SauE15.A6 | ?-529178 | 2725 pb | | 1281-1421 | (1477; 1476) | | |
| 93 | SauE4.I2 | — | 4577 pb | | 40-469 | (1484; 1483) | + | |
| 94 | SauE15.G1 | — | 2449 pb | | 1597-1738 | (1503; 1502) | | |
| 94 | SauE15.J2 | — | 2449 pb | 6b | 870-1195 | — | | |
| 94 | TspE4.K12 | — | 2449 pb | | 1074-1176 | — | | |
| 95 | TspE4.H12 | — | 3424 pb | j (Not l) | 1-285 | — | | |
| 96 | TspE15.D7 | — | 2051 pb | | 158-396 | (1519; 1518) | | |
| 96 | TspE4.L3 | — | 2051 pb | | 1-153 | (1519; 1518) | + | |
| 96 | SauE15.B1 | — | 2051 pb | | 1225-1412 | (1521; 1520) | + | |
| 97 | TspE4.C4 | 3597458-? | 364 pb | 4 | 145-325 | — | | |
| 98 | TspE4.F6 | — | 3624 pb | 3 | 2978-3221 | (1531; 1530) | + | |
| 99 | SauE15.C9 | 3833958-3835850 | 2286 | | 561-833e | (1536; 1535) | | |
| 99 | TspE4.G12 | 3833958-3835850 | 2286 | Not k | 1916-2089 | (1540; 1539) | + | |
| 100 | TspE4.G8 | — | 9416 pb | | 2188-2510 | (1543; 1542)(1545; 1544) | | + |
| 101 | SauE15.G9 | — | 2748 pb | 3 | 1654-1816 | (1564; 1563) | | |
| 101 | SauE4.D1 | — | 2748 pb | | 1048-1350 | (1564; 1563) | | |
| 48 | SauE15.H8 | 2068492-383159 | 5586 | | 1221-1528 | (721; 720) | | |
| | TspE4.J6 | 3652844-3652880 | | a (Not I) | 993-1153 | (730, 729)(732; 731) | | + |
| | SauE15.I11 | 3652844-3652880 | | | 2510-2671 | (732; 731) | | |
| | SauE4.E3 | 3652844-3652880 | | | 6146-6408 | (738; 737) | | |
| | TspE4.H2 | 3774655-3774666 | | u (Not I) | 547-749 | (747; 746) | | |
| | SauE15.L6 | 2067936-2774015 | 7977 | 6a | 1064-1330 | (752; 751) | | |
| | SauE4.H10 | 2067936-2774015 | | | 2947-3178 | — | | |
| | SauE4.E5 | 2067936-2774015 | | | 863-1059 | (752; 751) | | |
| | TspE15.H11 | 2067936-2774015 | | | 3069-3356 | — | | |
| | SauE15.N1 | 2067936-2774015 | | 6a | 5749-5951 | (752; 751) | | |
| | TspE4.G12 | 2076461-3835850 | 2334 | k (Not I) | 1706-1878 | (773; 772) | | |
| | sauE15.B12 | 2945708-? | 28820 | | 691-1196 | (776; 775)(778; 777) | | + |
| | SauE15.E10 | 2945708-? | | | 25007-25161 | (806; 805)(808; 807) | | + |
| 54 | SauE15.A11 | 1588103-1588103 | | c (NotI) | 1004-1128 | (817; 816) | | |
| 55 | SauE15.I1 | 1413657-1182865 | 2179 | | 442-583 | (824; 823) | | |
| 56 | TspE4.G1 | 3273109-239363 | | 1 | 47800-47958 | — | | |
| 57 | TspE15.E8 | 316624-316663 | 726 | | 220-380 | — | | |
| 58 | SauE15.F9 | 2056048-2060342 | | | 2445-2581 | (929; 928) | | |
| 58 | SauE15.H5 | 2056048-2060342 | | | 5839-6038 | (933; 932) | | |
| 58 | TspE4.F10 | 2056048-2060342 | | r (Not I) | 9684-9948 | (935; 934) | | |
| 58 | SauE4.E6 | 2056048-2060342 | | | 16929-17038 | (941; 940) | | |
| 58 | SauE4.C11 | 2056048-2060342 | | | 25143-25397 | (947; 946) | | |
| 58 | SauE15.M8 | 2056048-2060342 | | | 28203-28351 | (947; 946) | | |
| 58 | SauE4.H1 | 2056048-2060342 | | | 32821-33005 | (955; 954) | | |
| 58 | TspE4.A11 | 2056048-2060342 | | | 43906-44194 | (961; 960) | | |
| 58 | SauE4.C6 | 2056048-2060342 | | | 48042-48155 | (971; 970) | + | |
| 59 | SauE4.F10 | 1643312-1646494 | | | 3555-3795 | (990; 989)(992; 991) | | + |
| 60 | SauE15.L6 | ?-2068295 | | 6a | | | | |
| 61 | SauE4.B1 | 1425451-? | | | | | | |

TABLE 9

| | | Sequence | | | | | | | | |
|---|---|---|---|---|---|---|---|---|---|---|
| | | homolog | Identity | | Sequence | Identity | | Sequence | Identity | |
| | | E. coli to | (DNA | | homolog to | (DNA | | homolog to E. | (DNA | |
| fragment | Size (in bp) | RS218 | level) (%) | expect | coli O157: H7 | level) (%) | expect | E.coli CFT073 | level) (%) | expect |
| 1 | 1312 | 18-1311 | 99 | 0.0 | — | — | — | 1-1312 | 100 | 0.0 |
| 2 | 7316 | 4-7315 | 96 | 0.0 | 10-7315 | 96 | 0.0 | 1-7316 | 100 | 0.0 |
| 3 | 5203 | 1781-5094 | 99 | 0.0 | — | — | — | 1-5203 | 100 | 0.0 |
| 4 | 1584 | 1-1585 | 98 | 0.0 | — | — | — | 1-1584 | 100 | 0.0 |
| 5 | 2444 | 1-2443 | 99 | 0.0 | 1811-1926 | 92 | 1E−33 | 1-2444 | 100 | 0.0 |
| 5 | 2444 | | | | | | | 1-2444 | 100 | 0.0 |
| 6 | 8644 | 323-3755 | 99 | 0.0 | — | — | — | 1-8644 | 100 | 0.0 |
| | | 4926-6323 | 99 | 0.0 | — | — | — | | | |
| | | 6723-8643 | 99 | 0.0 | — | — | — | | | |
| 7 | 1835 | 1-1834 | 99 | 0.0 | 88-1834 | 95 | 0.0 | 1-1835 | 100 | 0.0 |
| 8 | 7648 | 1-7646 | 99 | 0.0 | — | — | — | 1-7648 | 100 | 0.0 |
| 8 | 7648 | | | | — | — | — | 1-7648 | 100 | 0.0 |
| 8 | 7648 | | | | — | — | — | 1-7648 | 100 | 0.0 |
| 8 | 7648 | | | | — | — | — | 1-7648 | 100 | 0.0 |
| 8 | 7648 | | | | — | — | — | 1-7648 | 100 | 0.0 |
| 9 | 9137 | 4-354 | 94 | 1E−148 | — | — | — | 1-9137 | 100 | 0.0 |
| | | 424-3186 | 98 | 0.0 | — | — | — | | | |
| | | 3899-5114 | 97 | 0.0 | — | — | — | | | |
| | | 5487-7279 | 99 | 0.0 | — | — | — | | | |
| | | 8553-9129 | 98 | 0.0 | — | — | — | | | |
| 10 | 665 | 1-666 | 99 | 0.0 | 5-665 | 98 | 0.0 | 1-665 | 100 | 0.0 |
| 11 | 4144 | 175-4046 | 95 | 0.0 | — | — | — | 1-4144 | 100 | 0.0 |
| 11 | 4144 | | | | — | — | — | 1-4144 | 100 | 0.0 |
| 11 | 4144 | | | | — | — | — | 1-4144 | 100 | 0.0 |
| 12 | 5426 | 1-5313 | 98 | 0.0 | — | — | — | 1-5426 | 100 | 0.0 |
| 13 | 8681 | 471-2118 | 96 | 0.0 | — | — | — | 1-8681 | 100 | 0.0 |
| 13 | 8681 | 2332-3189 | 97 | 0.0 | — | — | — | 1-8681 | 100 | 0.0 |
| | | 3374-8682 | 99 | 0.0 | — | — | — | | | |
| 14 | 9973 | 289-2790 | 98 | 0.0 | 93-427 | 82 | 1E−51 | 1-9973 | 100 | 0.0 |
| 14 | 9973 | 3531-5231 | 99 | 0.0 | | | | 1-9973 | 100 | 0.0 |
| | | 5408-9974 | 99 | 0.0 | | | | | | |
| 15 | 1370 | 1-1360 | 99 | 0.0 | 5-1360 | 97 | 0.0 | 1-1370 | 100 | 0.0 |
| 16 | 1692 | 698-1462 | 93 | 0.0 | 1193-1486 | 93 | E−117 | 1-1692 | 100 | 0.0 |
| 16 | 1692 | | | | | | | 1-1692 | 100 | 0.0 |
| 17 | 3761 | 1-308 | 94 | E−134 | — | — | — | 1-3761 | 100 | 0.0 |
| 17 | 3761 | | | | — | — | — | 1-3761 | 100 | 0.0 |
| 18 | 726 | 11-727 | 98 | 0.0 | — | — | — | 1-726 | 100 | 0.0 |
| 19 | 3270 | 1-310 | 98 | 1E−161 | — | — | — | 1-3270 | 100 | 0.0 |
| | | 569-1132 | 87 | 1E−158 | — | — | — | | | |
| | | 2285-3271 | 98 | 0.0 | — | — | — | | | |
| 20 | 4585 | 1-2302 | 98 | 0.0 | 4044-4197 | 91 | 2E−51 | 1-4585 | 100 | 0.0 |
| 20 | 4585 | 4014-4586 | 98 | 0.0 | | | | 1-4585 | 100 | 0.0 |
| 20 | 4585 | | | | | | | 1-4585 | 100 | 0.0 |
| 21 | 1951 | 1-875 | 98 | 0.0 | 5-1763 | 92 | 0.0 | 1-1951 | 100 | 0.0 |
| | | 1410-1952 | 95 | 0.0 | 1795-1929 | 88 | 3E−33 | | | |
| 22 | 2981 | 1-2980 | 96 | 0.0 | 4-2980 | 93 | 0.0 | 1-2981 | 100 | 0.0 |
| 23 | 1036 | -1035 | 98 | 0.0 | — | — | — | 1-1036 | 100 | 0.0 |
| 24 | 6318 | 865-6018 | 99 | 0.0 | 4-6317 | 96 | 0.0 | 1-6318 | 100 | 0.0 |
| 24 | 6318 | 6076-6317 | 99 | 1E−130 | | | | 1-6318 | 100 | 0.0 |
| 24 | 6318 | | | | | | | 1-6318 | 100 | 0.0 |
| 24 | 6318 | | | | | | | 1-6318 | 100 | 0.0 |
| 25 | 7325 | 1-7326 | 99 | 0.0 | — | — | — | 1-7325 | 100 | 0.0 |
| 26 | 712 | — | — | — | — | — | — | 1-712 | 100 | 0.0 |
| 27 | 13757 | 1-9589 | 98 | 0.0 | — | — | — | 1-13757 | 100 | 0.0 |
| 27 | 13757 | 9410-9681 | 85 | 6E−45 | — | — | — | 1-13757 | 100 | 0.0 |
| 27 | 13757 | 9724-13757 | 99 | 0.0 | — | — | — | 1-13757 | 100 | 0.0 |
| 28 | 12263 | 1-242 | 98 | 1E−125 | 1-416 | 96 | 0.0 | 1-12263 | 100 | 0.0 |
| 28 | 12263 | 126-410 | 93 | 1E−119 | 2480-2584 | 92 | 1E−33 | 1-12263 | 100 | 0.0 |
| 28 | 12263 | 396-6463 | 98 | 0.0 | 5668-5873 | 89 | 2E−60 | 1-12263 | 100 | 0.0 |
| 28 | 12263 | 6713-12264 | 98 | 0.0 | 10957-12264 | 96 | 0.0 | 1-12263 | 100 | 0.0 |
| 28 | 12263 | | | | | | | 1-12263 | 100 | 0.0 |
| 28 | 12263 | | | | | | | 1-12263 | 100 | 0.0 |
| 29 | 4871 | 697-814 | 88 | 7E−30 | 1503-3365 | 96 | 0.0 | 1-4871 | 100 | 0.0 |
| 29 | 4871 | 1128-1203 | 97 | 4E−31 | 702-808 | 87 | 3E−23 | 1-4871 | 100 | 0.0 |
| 29 | 4871 | 1503-4825 | 94 | 0.0 | 1128-1203 | 93 | 7E−24 | 1-4871 | 100 | 0.0 |
| 30 | 2777 | 1-2259 | 90 | 0.0 | 1943-2128 | 88 | 2E−47 | 1-2777 | 100 | 0.0 |
| 30 | 2777 | | | | | | | 1-2777 | 100 | 0.0 |
| 31 | 7655 | 1-3985 | 99 | 0.0 | 7195-7345 | 94 | 7E−59 | 1-7655 | 100 | 0.0 |
| 31 | 7655 | 4403-7654 | 99 | 0.0 | | | | 1-7655 | 100 | 0.0 |
| 32 | 6000 | 1-5999 | 97 | 0.0 | 1-252 | 99 | 1E−136 | 1-6000 | 100 | 0.0 |
| | | | | | 1290-1936 | 95 | 0.0 | | | |
| | | | | | 1950-2172 | 94 | 2E−94 | | | |
| | | | | | 2218-5309 | 93 | 0.0 | | | |
| | | | | | 5462-5999 | 89 | 1E−180 | | | |
| 33 | 2497 | 1-1880 | 97 | 0.0 | — | — | — | 1-2797 | 100 | 0.0 |
| | | 2120-2498 | 99 | 0.0 | — | — | — | | | |
| 34 | 3707 | 1-3708 | 99 | 0.0 | — | — | — | 1-3707 | 100 | 0.0 |
| 35 | 4821 | 1-4820 | 94 | 0.0 | — | — | — | 1-4821 | 100 | 0.0 |
| 36 | 7267 | 1-6977 | 99 | 0.0 | | | | 1-7267 | 100 | 0.0 |
| 36 | 7267 | 7009-7268 | 99 | 1E−143 | — | — | — | 1-7267 | 100 | 0.0 |
| 37 | 16066 | 1-15238 | 98 | 0.0 | — | — | — | 1-16066 | 100 | 0.0 |
| 37 | 16066 | 15594-16066 | 98 | 0.0 | — | — | — | 1-16066 | 100 | 0.0 |
| 37 | 16066 | | | | — | — | — | 1-16066 | 100 | 0.0 |
| 37 | 16066 | | | | — | — | — | 1-16066 | 100 | 0.0 |
| 38 | 5601 | 13-5602 | 99 | 0.0 | — | — | — | 1-5601 | 100 | 0.0 |
| 39 | 2703 | 128-2702 | 99 | 0.0 | 17-2702 | 93 | 0.0 | 1-2703 | 100 | 0.0 |
| 40 | 12101 | 1-10426 | 98 | 0.0 | — | — | — | 1-12101 | 100 | 0.0 |
| | | 10809-12102 | 97 | 0.0 | — | — | — | | | |
| 41 | 1423 | 1-1248 | 97 | 0.0 | — | — | — | 1-1423 | 100 | 0.0 |
| 42 | 3129 | 1-111 | 95 | 5E−45 | 1-1447 | 93 | 0.0 | 1-3129 | 100 | 0.0 |
| 43 | 5571 | 17-2829 | 93 | 0.0 | — | — | — | 1-5571 | 100 | 0.0 |
| | | 2959-5572 | 98 | 0.0 | — | — | — | | | |
| 44 | 8739 | 1-4516 | 98 | 0.0 | 121-7288 | 95 | 0.0 | 1-8739 | 100 | 0.0 |
| | | 4628-7434 | 96 | 0.0 | 7629-8729 | 90 | 0.0 | | | |
| | | 7611-8738 | 94 | 0.0 | | | | | | |
| 45 | 3245 | 1-900 | 99 | 0.0 | — | — | — | 1-3245 | 100 | 0.0 |
| 45 | 3245 | 1859-3244 | 97 | 0.0 | — | — | — | 1-3245 | 100 | 0.0 |
| 46 | 6164 | 1-6164 | 99 | 0.0 | — | — | — | 1-6164 | 100 | 0.0 |
| 46 | 6164 | | | 0.0 | — | — | — | 1-6164 | 100 | 0.0 |
| 47 | 1478 | 1-97 | 99 | 0.0 | — | — | — | 1-1478 | 100 | 0.0 |
| | | 320-1477 | 99 | 0.0 | — | — | — | | | |
| 48 | 5586 | 1-341 | 99 | 0.0 | 1-1965 | 98 | 0.0 | 1-5586 | 100 | 0.0 |
| | | 2018-5585 | 98 | 0.0 | 2023-2417 | 86 | 1E−102 | | | |
| | | | | | 2448-3811 | 89 | 0.0 | | | |
| 49 | 9054 | 1-5807 | 99 | 0.0 | 1-9054 | 96 | 0.0 | 1-9054 | 100 | 0.0 |
| 49 | 9054 | 7084-9054 | 98 | 0.0 | | | | | | |
| 49 | 9054 | | | | | | | | | |
| 50 | 6678 | 1-1419 | 93 | 0.0 | 1249-1432 | 85 | 2e−42 | 1-6678 | 100 | 0.0 |
| | | 2753-3219 | 93 | 0.0 | 4529-4800 | 91 | 1e−97 | | | |
| | | 5276-6678 | 97 | 0.0 | 5114-5520 | 83 | 1e−75 | | | |
| | | | | | 5595-6627 | 90 | 0.0 | | | |
| 51 | 7977 | 1509-3178 | 85 | 0.0 | 1-149 | 89 | e−48 | 1-7977 | 100 | 0.0 |
| 51 | 7977 | 5018-8184 | 97 | 0.0 | 346-775 | 89 | e−129 | | | |
| 51 | 7977 | | | | 7870-8184 | 86 | 2e−71 | | | |
| 51 | 7977 | | | | | | | | | |
| 51 | 7977 | | | | | | | | | |
| 52 | 2334 | 1-698 | 89 | 0.0 | 1-821 | 92 | 0.0 | 1-2334 | 100 | 0.0 |
| | | 978-2075 | 97 | 0.0 | | | | | | |
| 53 | 28820 | 57-2414 | 98 | 0.0 | — | — | — | 1-28820 | 100 | 0.0 |
| 53 | 28820 | 2691-4828 | 96 | 0.0 | | | | | | |
| | | 5297-11491 | 94 | 0.0 | | | | | | |
| | | 17104-18095 | 95 | 0.0 | | | | | | |
| | | 18785-20658 | 93 | 0.0 | | | | | | |
| | | 20826-28816 | 96 | 0.0 | | | | | | |
| 54 | 3281 | 1-282 | 99 | e−156 | 3-3279 | 96 | 0.0 | 1-3281 | 100 | 0.0 |
| | | 1086-3281 | 99 | 0.0 | | | | | | |
| 55 | 2179 | 9-617 | 99 | 0.0 | 1-2177 | 99 | 0.0 | 1-2179 | 100 | 0.0 |
| 56 | 48255 | 1-757 | 92 | 0.0 | 157-282 | 89 | 7e−34 | 1-48255 | 100 | 0.0 |
| | | 16613-16737 | 99 | 1e−61 | 327-761 | 84 | 2e−92 | | | |
| | | 17612-17835 | 85 | 2e−44 | 897-10117 | 97 | 0.0 | | | |
| | | 18068-18386 | 89 | e−103 | 11910-15258 | 94 | 0.0 | | | |
| | | 19103-20896 | 90 | 0.0 | 10376-10482 | 92 | 2e−34 | | | |
| | | 31049-32357 | 94 | 0.0 | 31049-32358 | 96 | 0.0 | | | |
| | | 33012-34582 | 91 | 0.0 | 48126-48205 | 91 | 9e−21 | | | |
| | | 47503-48254 | 95 | 0.0 | | | | | | |
| 57 | 726 | 1-561 | 98 | 0.0 | 1-561 | 96 | 0.0 | 1-726 | 100 | 0.0 |
| 58 | 48732 | 1-19781 | 98 | 0.0 | 46834-46991 | 92 | 2e−55 | 1-48732 | 100 | 0.0 |
| 58 | 48732 | 20058-24723 | 95 | 0.0 | | | | | | |
| 58 | 48732 | 24876-32641 | 98 | 0.0 | | | | | | |
| 58 | 48732 | 32783-40366 | 99 | 0.0 | | | | | | |
| 58 | 48732 | 41090-47187 | 97 | 0.0 | | | | | | |
| 58 | 48732 | 47568-48715 | 98 | 0.0 | | | | | | |
| 58 | 48732 | | | | | | | | | |
| 58 | 48732 | | | | | | | | | |
| 58 | 48732 | | | | | | | | | |
| 59 | 4810 | 49-173 | 96 | 9e−51 | 49-197 | 91 | 2e−48 | 1-4810 | 100 | 0.0 |
| | | 1812-3795 | 94 | 0.0 | 1812-2707 | 89 | 0.0 | | | |
| | | | | | 2840-3797 | 82 | <e−100 | | | |
| | | | | | 4676-4809 | 94 | 6e−49 | | | |
| 60 | 27324 | 1472-1908 | 92 | e−172 | 1907-3997 | 93 | 0.0 | 1-27324 | 100 | 0.0 |
| | | 3136-4010 | 83 | <e−100 | | | | | | |
| 61 | 5207 | 1-5203 | 95 | 0.0 | 1-106 | 88 | 5e−25 | 1-5207 | 100 | 0.0 |
| | | | | | 230-4088 | 94 | 0.0 | | | |
| | | | | | 4318-5203 | 88 | 0.0 | | | |
| 62 | 16244 | 2-452 | 94 | 0.0 | 2-497 | 93 | 0.0 | 1-16244 | 100 | 0.0 |
| 62 | 16244 | 584-1292 | 93 | 0.0 | 612-1052 | 93 | 0.0 | | | |
| 62 | 16244 | 1396-4430 | 97 | 0.0 | 6607-7916 | 96 | 0.0 | | | |
| 62 | 16244 | 6606-7916 | 93 | 0.0 | 14096-15407 | 96 | 0.0 | | | |
| | | 8396-8985 | 88 | 2e−168 | | | | | | |
| | | 9117-9968 | 88 | 0.0 | | | | | | |
| | | 14100-15407 | 94 | 0.0 | | | | | | |
| 63 | 5307 | 1-2863 | 99 | 0.0 | — | — | — | 1-5307 | 100 | 0.0 |
| | | 4182-5307 | 99 | 0.0 | | | | | | |
| 64 | 6202 | 53-880 | 96 | 0.0 | 53-1042 | 94 | 0.0 | 1-6202 | 100 | 0.0 |
| | | 1111-6921 | 99 | 0.0 | | | | | | |
| 65 | 12133 | 1-1894 | 95 | 0.0 | 1518-1868 | 81 | 2e−44 | 1-12133 | 100 | 0.0 |
| | | 6071-8577 | 95 | 0.0 | 7357-7808 | 80 | e−44 | | | |
| | | 10442-11371 | 92 | 0.0 | | | | | | |
| 66 | 41818 | 1-926 | 93 | 0.0 | 9126-9752 | 97 | 0.0 | 1-41818 | 100 | 0.0 |
| 66 | 41818 | 1111-1758 | 95 | 0.0 | 19485-20595 | 91 | 0.0 | | | |
| | | 1894-2366 | 88 | <e−87 | 21340-21578 | 90 | 5e−78 | | | |
| | | 2812-3115 | 88 | 2e−87 | 26296-26624 | 89 | e−104 | | | |
| | | 3314-3798 | 90 | e−160 | 30651-31490 | 88 | 0.0 | | | |
| | | 7211-7637 | 89 | e−125 | | | | | | |
| | | 26291-26624 | 89 | <e−103 | | | | | | |
| | | 30676-31490 | 90 | 0.0 | | | | | | |
| | | 32016-32675 | 97 | 0.0 | | | | | | |
| | | 32950-34421 | 97 | 0.0 | | | | | | |
| | | 35370-38907 | 97 | 0.0 | | | | | | |
| | | 38942-41136 | 98 | 0.0 | | | | | | |
| | | 38942-41808 | 95 | 0.0 | | | | | | |
| 67 | 1374 | 4-300 | 95 | e−113 | — | — | — | 1-1374 | 100 | 0.0 |
| | | 366-1371 | 98 | 0.0 | — | — | — | | | |
| 68 | 15368 | 1-1548 | 99 | 0.0 | 8815-9112 | 95 | e−34 | 1-15368 | 100 | 0.0 |
| 68 | 15368 | 1714-3699 | 98 | 0.0 | 9559-10669 | 93 | 0.0 | | | |
| | | 4264-15368 | 99 | 0.0 | 10749-13990 | 97 | 0.0 | | | |
| | | | | | 15226-15322 | 94 | 7e−136 | | | |
| 69 | 16373 | 653-1938 | 95 | 0.0 | 39-465 | 88 | e−126 | 1-16373 | 100 | 0.0 |
| 69 | 16373 | 10000-10275 | 91 | e−100 | 2818-3271 | 94 | 0.0 | | | |
| 69 | 16373 | 11917-13696 | 87 | 0.0 | 10920-13762 | 93 | 0.0 | | | |
| 69 | 16373 | | | | | | | | | |
| 70 | 1350 | 834-1350 | 98 | 0.0 | — | — | — | 1-1350 | 100 | 0.0 |
| 70 | 1350 | | | | | | | | | |
| 71 | 11475 pb | 1-11475 | 100 | 0.0 | — | — | — | — | — | — |
| 71 | 11475 pb | | | | | | | | | |
| 71 | 11475 pb | | | | | | | | | |
| 72 | 4576 pb | 1-4576 | 100 | 0.0 | — | — | — | — | — | — |
| 72 | 4576 pb | | | | | | | | | |
| 72 | 4576 pb | | | | | | | | | |
| 72 | 4576 pb | | | | | | | | | |
| 73 | 1906 pb | 1-1906 | 100 | 0.0 | 436-1127 | 91% | 0.0 | 437-1127 | 91% | 0.0 |
| | | | | | 1207-1507 | 88% | 2e-84 | 1207-1507 | 87% | 2e−80 |
| 74 | 2536 pb | 1-2536 | 100 | 0.0 | 0-2536 | 98% | 0.0 | — | — | — |
| 75 | 894 pb | 1-894 | 100 | 0.0 | 659-852 | 81% | 5e−21 | — | — | — |
| 76 | 747 pb | 1-747 | 100 | 0.0 | 15-747 | 98% | 0.0 | — | — | — |
| 77 | 886 pb | 1-886 | 100 | 0.0 | — | — | — | — | — | — |
| 78 | 4892 pb | 1-4892 | 100 | 0.0 | — | — | — | — | — | — |
| 79 | 6927 pb | 1-6927 | 100 | 0.0 | — | — | — | — | — | — |
| 80 | 10848 pb | 1-10848 | 100 | 0.0 | 3036-4346 | 95% | 0.0 | 3037-4346 | 96% | 0.0 |
| | | | | | | | | (IS629) | | |
| 80 | 10848 pb | | | | 2834-3001 | 84% | 4e−24 | | | |
| 81 | 1716 pb | 1-1716 | 100 | 0.0 | 1174-1387 | 94% | 6e−93 | 1175-1358 | 94% | 7e−77 |
| 82 | 200 pb | 1-200 | 100 | 0.0 | — | — | — | — | — | — |
| 83 | 5873 pb | 1-5873 | 100 | 0.0 | — | — | — | 1-991 | 95% | 0.0 |
| 83 | 5873 pb | | | | | | | | | |
| 83 | 5873 pb | | | | | | | | | |
| 84 | 4737 pb | 1-4737 | 100 | 0.0 | — | — | — | — | — | — |
| 84 | 4737 pb | | | | | | | | | |
| 84 | 4737 pb | | | | | | | | | |
| 85 | 4386 pb | 1-4386 | 100 | 0.0 | 1-584 | 96% | 0.0 | 217-625 | 89% | e−132 |
| 85 | 4386 pb | | | | 4252-4379 | 89% | 3e−29 | 4249-4379 | 87% | 4e−28 |
| 86 | 5819 pb | 1-5819 | 100 | 0.0 | 3431-3882 | 82% | 3e−76 | 3383-5819 | 95% | 0.0 |
| 86 | 5819 pb | | | | 5417-5571 | 85% | 6e−28 | | | |
| 86 | 5819 pb | | | | | | | | | |
| 87 | 6066 pb | 1-6066 | 100 | 0.0 | 664-1166 | 83% | 9e−95 | — | — | — |
| 87 | 6066 pb | | | | 3049-3625 | 81% | 1e−81 | | | |
| 87 | 6066 pb | | | | 2628-2885 | 84% | 1e−44 | | | |
| | | | | | 361-545 | 83% | 2e−24 | | | |
| 88 | 7890 pb | 1-7890 | 100 | 0.0 | 1-315 | 86% | 2e−71 | 1-3167 | 97% | 0.0 |
| 88 | 7890 pb | | | | | | | 5813-6648 | 96% | 0.0 |
| 88 | 7890 pb | | | | | | | 5381-5699 | 91% | e−114 |
| 88 | 7890 pb | | | | | | | 5078-5348 | 89% | 4e−85 |
| 88 | 7890 pb | | | | | | | 4798-4953 | 81% | 2e−25 |
| 89 | 5662 pb | 1-5622 | 100 | 0.0 | 3000-3323 | 85% | 1e−69 | 2775-3323 | 93% | 0.0 |
| 89 | 5662 pb | | | | 2271-2617 | 80% | 2e−37 | 3324-3403 | 91% | 2e−21 |
| 89 | 5662 pb | | | | | | | | | |
| 89 | 5662 pb | | | | | | | | | |
| 89 | 5662 pb | | | | | | | | | |
| 89 | 5662 pb | | | | | | | | | |
| 89 | 5662 pb | | | | | | | | | |
| 90 | 1314 | 1-1314 | 100 | 0.0 | — | — | — | — | — | — |
| 90 | 1314 | | | | | | | | | |
| 91 | 1581 pb | 1-1581 | 100 | 0.0 | — | — | — | — | — | — |
| 92 | 2725 pb | 1-2725 | 100 | 0.0 | 160-2355 | 96% | 0.0 | 1-2483 | 94% | 0.0 |
| | | | | | 2362-2483 | 98% | 1e−58 | 2585-2652 | 95% | 5e−24 |
| 93 | 4577 pb | 1-4577 | 100 | 0.0 | — | — | — | — | — | — |
| 94 | 2449 pb | 1-2449 | 100 | 0.0 | — | — | — | 1-415 | 94% | 0.0 |
| 94 | 2449 pb | | | | | | | 794-1255 | 95% | 0.0 |
| 94 | 2449 pb | | | | | | | 555-632 | 98% | 6e−35 |
| 95 | 3424 pb | 1-3424 | 100 | 0.0 | 5-278 | 95% | e−123 | — | — | — |
| 96 | 2051 pb | 1-2051 | 100 | 0.0 | — | — | — | — | — | — |
| 96 | 2051 pb | | | | | | | | | |
| 96 | 2051 pb | | | | | | | | | |
| 97 | 364 pb | 1-364 | 100 | 0.0 | — | — | — | — | — | — |
| 98 | 3624 pb | 1-3624 | 100 | 0.0 | — | — | — | 1-918 | 96% | 0.0 |
| 99 | 2286 | 1-2286 | 100 | 0.0 | — | — | — | 1188-2286 | 97% | 0.0 |
| 99 | 2286 | | | | | | | | | |
| 100 | 9416 pb | 1-9416 | 100 | 0.0 | — | — | — | — | — | — |
| 101 | 2748 pb | 1-2748 | 100 | 0.0 | — | — | — | — | — | — |
| 101 | 2748 pb | | | | | | | | | |

Apart from zone 24 and its orfs/ORFs which correspond to known products which are of known nature B2+A−, none of the sequences identified herein is strictly identical to a sequence of the prior art (comparison of the nucleotide series over their entire length, independently of the function of the process). However, when comparing the complete sequence of these CFT073+K12− zones and RS218+ K12− zones with those indicated for the E. coli strain O157:H7 (strain of phylogenic group not clearly determined, responsible for infections located in the intestine), some homologies are found as it appears from table 9 above.

With regard now to the ORFs which were identified on each of said zones, the percentage identity (% id) and percentage similarity (% sim) obtained at the polypeptide level by comparison on databases are given in FIG. 6.

This being so, for each of the CFT073+ K12− zones isolated herein, and for each of the orfs and ORFs identified herein (except zone 24 and its orfs/ORFs) as well as for each of the isolated RS218+/K12− zones, it is the first description of a nature CFT073+ K12− or RS218+/K12− respectively. These zones, orfs and ORFs therefore have, at the very least, applications in the domain of phylogeny (diagnostic applications) since these products make it possible to distinguish between two E. coli strains with very different pathogenicities. Anti-CFT073 or anti-RS218 vaccine applications might also be envisaged.

In addition, since the CFT073+ K12− and RS218+/K12− zones isolated herein all comprise, in the vicinity of their chromosome, at least one DNA fragment which is of nature B2/D+ A− as defined above (ECOR B2 frequency and/or ECOR D frequency higher than ECOR A frequency SEQ ID NO 1 to NO. 550, the zones and orfs isolated herein are excellent candidates as polynucleotides which are of nature B2/D+ A−. Those of the CFT073+ K12− ORFs and orfs which comprise in their sequence a clone which is of nature B2/D+ A− as defined in Example 1 above (SEQ ID NO 1 to NO 253) have an even higher probability of being products which are of nature B2/D+ A−.

Verifying that the CFT073+ K12− or RS218+/K12− zones, ORFs and orfs isolated herein are effectively of nature B2/D+ A− can be easily carried out by those skilled in the art according to techniques which are conventional in the domain of phylogeny. One way of making sure may, for example, consists in applying to at least one other E. coli strain of group B2 or D, for example E. coli C5, the procedure which has been described herein for the E. coli strain CFT073 and RS218, in such a way as to identify and isolate the zones, ORFs and orfs which are present in E. coli C5 and absent in E. coli K12 (or another E. coli strain of group A). The sequences of these C5+ K12− zones, ORFs and orfs are then compared with those of the CFT073+ K12− and RS218+ K12− groups described herein, while searching for homologous sequences and sequence fragments. This comparison can, for example, be carried out using the BLAST program (National Centre for Biotechnology Information, NCBI, Altschul et al. 1997, Nucleic Acids Res. 25: 3389-3402), comparing each sequence of the C5+ K12− group with those of the CFT073+ K12− (or RS218+ K12−) group. The polynucleotide sequences, or fragments of polynucleotide sequence, with a low probability of the significant homology (for example, identity of 80% or more) being linked by chance can then be selected as being of nature B2/D+ A−, as defined above. If desired, the process can be repeated on a fourth or fifth E. coli strain of group B2 or D.

Such B2/D+ A− products are particularly useful for the phylogenic determination of E. coli.

The polynucleotides which are of nature B2/D+ A− and their inactivated isogenic mutants can be used, in the form of isolated DNAs (placed under the control of a eukaryotic promoter or in the form of DNA transfected into a cell), as active principles in a vaccine composition intended to prevent, alleviate or combat an undesirable development of E. coli, and in particular a development of E. coli in an extra-intestinal compartment. The ORFs themselves, administered in an optionally inactivated and immunogenic form, are active principles which are very promising for the development of a vaccine composition intended to prevent, alleviate or combat an undesirable development of E. coli, and in particular a development of E. coli in an extra-intestinal compartment.

The polynucleotides and polypeptides which are of nature B2/D+ A− can also be used as anti-pathogenicity targets (with extra-intestinal and non intra-intestinal targeting); they make it possible to identify and isolate capable active principles which can be used in pharmaceutical compositions (medicinal products in particular) intended to inhibit the growth of an E. coli bacterium and, notably, its extra-intestinal development, by selecting, from candidate active principles, those which inhibit or block the correct transcription and/or translation of these zones or orfs, or which inhibit or block the activity of the ORFS.

The present application is aimed towards each of these CFT073+ K12− and RS218+/K12− zones, numbers 1 to 23, 25 to 48, and 49 to 101, also their ORFs and orfs, as products. It is also aimed towards any vaccine or pharmaceutical composition intended to prevent, alleviate or combat an undesirable development of E. coli, and in particular an extra-intestinal development of E. coli, which comprises at least one of said zones or at least one of said orfs and ORFs.

Particularly targeted are the zones, ORFs and orfs which are both CFT073+ K12− and RS218+ K12−. When the applications targeted concern a development of E. coli in an extra-intestinal compartment (systemic and non-diarrhoeal E. coli infection), those of said CFT073+ K12−, RS218+ K12− zones, orfs and ORFs which are also O157:H7− are then preferred.

The present application is thus directed towards any isolated polynucleotide the sequence of which can be obtained by:

i. isolating a set of polynucleotide sequences of a strain of group B2 or D (such as E. coli RS218, E. coli CFT073 or E. coli C5) by:

locating, on the chromosome of this E. coli strain of group B2, sequences which exhibit, with the clones of SEQ ID NO 1-153 and 170-253, homology such that the probability of this homology being due to chance is very low, for example an homology above 80% of identity and

sequencing the chromosome of this E. coli strain of group B2 or D in the 5′ and 3′ directions, using each of the homologous sequences located, and stopping the sequencing as soon as a sequence is reached which exhibits significant homology above 80% of identity with a sequence contained in the chromosome of an E. coli strain of group A, such as E. coli K12,

ii. repeating the operation indicated in i. above on at least one other E. coli strain of group B2 or D, then

iii. comparing the sequences obtained for each E. coli of group B2 or D tested in such as way as to search for the sequences and sequence fragments which are homologous, among the various E. coli strains of group B2 or D tested (for example, using a program such as BLAST).

iv. selecting and isolating the sequences and sequence fragments which exhibit significant homology above 80% of identity, of the homology measured between a sequence or sequence fragment derived from the set obtained in i. and a sequence or sequence fragment obtained in ii. being due to chance).

The present application is also aimed towards the possible orfs identifiable on such a polynucleotide, and the ORFs and polypeptides encoded by these orfs.

The present application is also aimed towards the phylogenic, diagnostic and therapeutic applications and uses of these products, as indicated above.

### EXAMPLE 7

Therapeutic Application of the Novel B2/D+ A− Products

The B2/D+ A− DNAs isolated according to the invention are useful as active principles in the context of a vaccine composition intended to prevent, alleviate or combat an undesirable development of E. coli, and in particular a development of E. coli in an extra-intestinal compartment (systemic and non-diarrhoeal E. coli infections). They can then be used in an isolated (“naked” DNA) form or in the form of DNA transfected into a cell chosen for its physiological innocuity and its capacity to secrete the polypeptide encoded by the transfected B2/D+ A− fragment.

The polypeptides encoded by the DNAs which are of nature B2/D+ A− can also be used as active principles in the context of such vaccine compositions. They are then used in an inactivated and immunogenic form.

Examples of extra-intestinal development of E. coli comprise, in particular, septicaemias, pyelonephritis, and meningeal infections in newborns. Recognized or potential hospital-acquired infections are most commonly the product of such extra-intestinal developments of E. coli.

For extra-intestinal applications, those of the DNAs and of the polypeptides which are of nature B2/D+ A− and which are not present in the E. coli strains responsible for infections located in the intestine, such as E. coli O157:H7, and/or the transcripts of which are increased in the serum by comparison with a culture on standard nutrient medium (cf. Example 3 above), are more particularly preferred.

Region 5 (cf. in particular previous examples) appears to be an advantageous source for manufacturing broad-spectrum anti-B2-coli products. As regards regions 1, 3 and 4, they appear to be advantageous sources for manufacturing products against septicaemia with meningitis and in particular against meningitis in newborns and in adults.

The invention is aimed in particular towards the compositions, in particular the vaccine and pharmaceutical compositions, comprising at least one of said novel DNAs or at least one of said novel ORFs or at least one polypeptide the sequence of which can be considered to correspond, according to the universal genetic code and taking into account the degeneracy of this code, to one of these DNAs or ORFs. It is also aimed towards the use of the known DNAs which are of novel nature B2/D+ A−, such as chuA, the chuA fragments which are of nature B2/D+ A−, and of the polypeptides corresponding to these DNAs, for manufacturing such compositions.

Alternatively, the DNAs or polypeptides which are of nature B2/D+ A− can be used for screening chemical and/or biological libraries in such a way as to identify compounds capable of binding to them and of blocking an undesirable development of E. coli bacteria (by blocking the correct transcription and/or translation of these DNAs, or by blocking the activity of the polypeptides which they encode).

The formulation and the dose of such compositions can be developed and adjusted by those skilled in the art as a function of the medical indication targeted, of the method of administration desired, and of the patient under consideration (age, weight, sex, condition).

These compositions can also comprise one or more physiologically inert vehicles, and in particular any excipient suitable for the pharmaceutical formulation and/or for the method of administration desired (tablet, patch, gelatin capsule, powder, spray, drinkable solution, injectable solution, colloid). They can also comprise one or more active co-agent(s) in order to modify the intensity of activity of the B2/D+ A− compound according to the invention, or to modify its system of activity, or alternatively to modify the targeting of its activity. They can also comprise other agents useful for preventing and/or alleviating and/or treating an infection, without these agents having any interaction with the B2/D+ A− compounds according to the invention.

Preferably, the polynucleotides and polypeptides which are of nature B2/D+ A− according to the invention are used for identifying, for example by screening chemical and/or biological libraries, compounds capable of inhibiting, in vivo or under conditions mimicking as closely as possible the in vivo state, the activity of a protein the ORF of which comprises a polynucleotide which is of nature B2/D+ A− according to the invention. Such compounds can, in particular, be used in compositions, in particular pharmaceutical compositions, intended to inhibit the growth of E. coli, and in particular its extra-intestinal growth. A subject of the present invention is such an identification method, the compounds as obtained by such a method, and such compositions.

### EXAMPLE 8

Animal Models for Studying E. coli Virulence

1. Newborn rats (Sprague Dawley—January breeding) after 24 hours of acclimatisation in animal houses were infected when 5 days old. The injected inoculum was prepared from dilutions in physiological serum in a nutritive culture medium of 2 h. The animals were infected by intra-peritoneal route after anesthesia with ether, then put back to their mother after randomization by brood of ten.

The numeration of the bacteraemia at 18 h was obtained by taking of 5 μl otf blood after incision of the tail.

Bacteraemia at H18 (%) of 4 days old rats after intra peritoneal injection of various strains of E. coli.

| | | INOCULUM | | |
|---|---|---|---|---|
| GROUP | STRAIN | 100 bacteria | 10000 bacteria | 1000000 bacteria |
| B2 | C5 | 100% | ND* | ND* |
| B2 | CFT073 | 0% | 0% | all dead 100% |
| A | S82 | 0% | 0% | 66% |
| D | S16 | 25% | 64% | ND* |

*ND not done

2. Mortality of adult mice (%) at D7 after intra peritoneal injection of 3 strains of E. coli. The inoculum was prepared as above-mentioned and the injection made by the intra peritoneal route.

| | | INOCULUM | | |
|---|---|---|---|---|
| | | | | 10⁶ to 10³ |
| GROUP | STRAIN | 10⁸ bacteria | 10⁷ bacteria | bacteria |
| B2 | C5 | 100% | 100% | 0% |
| B2 | CFT073 | 100% | 0% | 0% |
| A | ECOR4 | 0% | 0% | 0% |

It appears from said results that the E. coli B2 responsible for neonatal infection (C5 strain) are capable inducing a bacteraemia after injection of a weak inoculum in the newborn rat and that the mortality in mice is 10 times higher. With CFT073, i.e. the E. coli B2 responsible for sepsis in the adult, it is necessary to use a stronger inoculum. But said coli is still pathogenic in mice. An E. coli of group A is not pathogenic even in mice.

With the claimed sequences, it is then possible to generate mutants whose virulence can be tested on said animal models.

1. A library of DNA fragments of E. coli strains consisting polynucleotides having a nature B2/D⁺ A⁻.

2. The library according to claim 1, selected from the group comprising the E. coli C5⁺ A⁻ library, the E. coli CFT073+ K12− library, the E. coli RS218+ K12− library, or the E. coli CFT073+ K12− and RS218+ K12− library.

3. The library according to claim 1 or 2, which is devoid of 0157:H7− polynucleotides.

4. A method for isolating from E.coli strain(s) the entire set of the chromosomal and plasmid polynucleotides having a nature B2/D⁺ A⁻, comprising subtracting the polynucleotide population of one or more E. coli strain(s) of group A, randomly sheared, from the polynucleotide population of one or more E. coli strain(s) of group B2 or D, cleaved with a restriction endonuclease allowing fragments comprising from 100 to 1500 bp approximately, with a mean of 300 to 500 bp approximately, to be produced, said subtraction being iteratively repeated, if desired.

5. The method of claim 4, wherein the E.coli strain submitted to said substraction is an E.coli C5 strain or an E. coli RS218 stain, and the E.coli strains of group A are of the ECOR collection.

6. Isolated polynucleotides having a nature B2/D⁺ A⁻, with a sequence selected from the group comprising:
the sequences SEQ ID N^{o}1 to 153,
the polynucleotide sequences obtainable by digesting the total DNA of an E. coli of group B2 or group D with a restriction enzyme, selecting those of the obtained fragments which hybridize with at least one sequence selected in the group consisting of SEQ ID N^{o} 134, 144, 109, 115, 140, 135, 33, 56, 122, 130, 141, 25, 48, 51, 57, 121,44, 45, 113, 119, 120, 23, 52, and
the sequences of the orfs containing one of said sequences,
the nature-conserving variant or fractional sequences of said sequences.

7. The polynucleotides of claim 6, the transcription of which is increased in the presence of human or animal serum and the genes bearing such polynucleotides.

8. Pair of primers allowing the amplification of a polynucleotide according to claim 6 or 7.

9. Pair of primers according to claim 8, corresponding to SEQ ID N^{o} 164 and 165.

10. Probes which are elaborated from a polynucleotide according to claim 6 or 7.

11. The use as a probe of a polynucleotide according to claim 6 or 7, such as obtained by amplification with a pair of primers selected from the group comprising SEQ ID N^{o} 160;161; SEQ ID N^{o}162,163; SEQ ID N^{o}164,165.

12. The polynucleotides having a sequence corresponding to the antisens of the polynucleotides according to claim 6 or 7.

13. A combination of a polypeptide encoded by a polynucleotide according to claim 6 or 7 with a ligand which is capable of inhibiting a biological function of E. coli, such as the extra-intestinal growth, said ligand being selected from the group comprising serum, purified serum, antibodies and monoclonal antibodies.

14. Vector comprising at least a polynucleotide according to claim 6, 7 or 11 and cells tranfected by such a vector.

15. The method for the phylogenic determination of an E.coli strain comprising the use of at least a polynucleotide isolated from the library according to anyone of claims 1 to 3.

16. The method of claim 15 wherein said polynucleotide is selected from the group comprising: a polynucleotide according to claim 6 or 7, or a polynucleotide selected from the group comprising:
the polynucleotides corresponding to the chuA gene and to its operon,
the polynucleotides the sequence of which corresponds to SEQ ID NO: 170, 171, 174, 175, 178, 179, 183, 185, 186, 190, 191, 193, 194, 195, 196, 199, 200, 202, 205, 206, 208, 209, 211, 214, 218, 220, 221, 233, 234, 235, 241, 244, 246, 247, 248 and 250,
the polynucleotides which can be obtained by digesting the total DNA of an E. coli of group B2 or D with a restriction enzyme chosen from the group consisting of NotI and BlnI, and selecting those of the fragments obtained which hybridize to at least one sequence chosen from the group consisting of SEQ ID NO: 125, 123, 116, 43, 40, 127, 133, 27, 34, 36, 42, 46, 54, 55, 38, 128 and 151, with the exception of the polynucleotides sfa, hly, cnf1, pap/prs, hra and ibe 10,
the polynucleotides the sequence of which corresponds to an orf comprising the sequence of one of the polynucleotides cited in the preceding three paragraphs,
the polynucleotides the sequence of which corresponds to a nature-conserving variant or fractional sequence of the sequence of at least one of these polynucleotides (and in particular SEQ ID NO: 241, 195, 185 and 248),
the pair of primers, the probes and the antisense polynucleotides, the sequence of which is as derived from these polynucleotides,
the binding products which are capable of binding to a polypeptide encoded by a polynucleotide which is of nature B2/D+ A−,

17. The method of claim 15 or 16, comprising the detection of yjaA.

18. The method of claim 17, wherein yjaA is detected with a probe, a pair of primers such as SEQ ID N^{o}162, 163, or the encoded protein is detected by a specific antibody.

19. A pharmaceutical composition, comprising an effective amount of a polynucleotide of the library of anyone of claims 1 to 3, or the encoded proteins in association with an inert carrier.

20. The pharmaceutical composition of claim 19, wherein said polynucleotide is according to claim 6 or 7, a vector or a cell according to claim 14 or an antisens according to claim 12.

21. The pharmaceutical composition of claim 20, wherein said polynucleotide(s) is (are) selected in the group comprising polynucleotides of SEQ ID NO: 71, 114, 13, 77, 8, 36, 120 and 130.

22. The pharmaceutical composition of anyone of claims 19 to 21, for treating and/or palliating and/or preventing extra-intestinal E.coli infections.

23. The use for making drugs for preventing or treating, E. coli extra-intestinal infections of at least one polynucleotide selected from the group comprising:
the polynucleotides corresponding to the chuA gene and to its operon,
the polynucleotides the sequence of which corresponds to SEQ ID NO: 170, 171, 174, 175, 178, 179, 183, 185, 186, 190, 191, 193, 194, 195, 196, 199, 200, 202, 205, 206, 208, 209, 211, 214, 218, 220, 221, 233, 234, 235, 241, 244, 246, 247, 248 and 250,
the polynucleotides which can be obtained by digesting the total DNA of an E. coli of group B2 or D with a restriction enzyme chosen from the group consisting of NotI and BlnI, and selecting those of the fragments obtained which hybridize to at least one sequence chosen from the group consisting of SEQ ID NO: 125, 123, 116, 43, 40, 127, 133, 27, 34, 36, 42, 46, 54, 55, 38, 128 and 151, with the exception of the polynucleotides sfa, hly, cnf1, pap/prs, hra and ibe 10,
the polynucleotides the sequence of which corresponds to an orf comprising the sequence of one of the polynucleotides cited in the preceding three paragraphs,
the polynucleotides the sequence of which corresponds to a nature-conserving variant or fractional sequence of the sequence of at least one of these polynucleotides (and in particular SEQ ID NO: 241, 195, 185 and 248),
the pair of primers, the probes and the antisense polynucleotides, the sequence of which is as derived from these polynucleotides,
the peptides encoded by said polynucleotides,
the binding products which are capable of binding to a polypeptide encoded by a polynucleotide which is of novel nature B2/D+ A−.

24. The use according to claim 23, wherein said polynucleotide(s) is (are) selected in the group comprising the polynucleotides pf SEQ ID NO 174, 202, 178, 221, 206, 175, 196, 170.

25. The pharmaceutical composition of anyone of claims 19 to 22 or the drug obtained according to claim 23 or 24, for treating septicaemia, pyelonephritis, and meningeal infections in newborns.

26. Use of the polynucleotides of the library of anyone of claims 1 to 3, or antisens thereof for the diagnostic of the presence or the absence of undesired E.coli such as contaminants E.coli or extra-intestinal E.coli. in a sample.

27. The use according to claim 26, wherein said DNA polynucleotide is according to claim 6 or 7, or is in a vector or a cell according to claim 14.

28. The use according to claim 27, wherein said polynucleotide is selected from the group comprising:
the polynucleotides corresponding to the chuA gene and to its operon,
the polynucleotides the sequence of which corresponds to SEQ ID NO: 170, 171, 174, 175, 178, 179, 183, 185, 186, 190, 191, 193, 194, 195, 196, 199, 200, 202, 205, 206, 208, 209, 211, 214, 218, 220, 221, 233, 234, 235, 241, 244, 246, 247, 248 and 250,
the polynucleotides which can be obtained by digesting the total DNA of an E. coli of group B2 or D with a restriction enzyme chosen from the group consisting of NotI and BlnI, and selecting those of the fragments obtained which hybridize to at least one sequence chosen from the group consisting of SEQ ID NO: 125, 123, 116, 43, 40, 127, 133, 27, 34, 36, 42, 46, 54, 55, 38, 128 and 151, with the exception of the polynucleotides sfa, hly, cnf1, pap/prs, hra and ibe 10,
the polynucleotides the sequence of which corresponds to an orf comprising the sequence of one of the polynucleotides cited in the preceding three paragraphs,
the polynucleotides the sequence of which corresponds to a nature-conserving variant or fractional sequence of the sequence of at least one of these polynucleotides (and in particular SEQ ID NO: 241, 195, 185 and 248),
the pair of primers, the probes and the antisense polynucleotides, the sequence of which is as derived from these polynucleotides,
the binding products which are capable of binding to a polypeptide encoded by a polynucleotide which is of novel nature B2/D+ A−.

29. A method for identifying a compound capable of inhibiting the extra-intestinal growth of E. coli, which comprises the detection of at least one compound:
i. capable of binding to at least one polynucleotide chosen from the group consisting of:
novel polynucleotides which are of novel nature B2/D+ A−, i.e.
the sequences SEQ ID NO: 1 to NO: 153,
the polynucleotide sequences which can be obtained by digesting the total DNA of an E. coli of group B2 or group D (such as, for example, an E. coli strain isolated from the blood of a patient suffering from a septicaemic or meningeal infection) with a restriction enzyme chosen from the group consisting of NotI and BlnI, selecting those of the fragments obtained which hybridize with at least one sequence chosen from the group consisting of SEQ ID NO: 134, 144, 109, 115, 140, 135, 33, 56, 122, 130, 141, 25, 48, 51, 57, 121, 44, 45, 113, 119, 120, 23, 52 and sequencing selected fragments, and
the sequences of the orfs (open reading frames) which contain one of these sequences,
the nature-conserving variant or fractional sequences of these sequences,
the polynucleotides corresponding to the chuA gene and to its operon,
the polynucleotides the sequence of which corresponds to SEQ ID NO: 170, 171, 174, 175, 178, 179, 183, 185, 186, 190, 191, 193, 194, 195, 196, 199, 200, 202, 205, 206, 208, 209, 211, 214, 218, 220, 221, 233, 234, 235, 241, 244, 246, 247, 248 and 250,
the polynucleotides which are as obtained by digesting the total DNA of an E. coli of group B2 or D with a restriction enzyme chosen from the group consisting of NotI and BlnI, and selecting those of the fragments obtained which hybridize to at least one sequence chosen from the group consisting of SEQ ID NO: 125, 123, 116, 43, 40, 127, 133, 27, 34, 36, 42, 46, 54, 55, 38, 128 and 151, with the exception of the polynucleotides sfa, hly, cnf1, pap/prs, hra and ibe 10,
the polynucleotides the sequence of which corresponds to an orf comprising the sequence of one of the polynucleotides cited in the preceding four paragraphs,
the polynucleotides the sequence of which corresponds to a nature-conserving variant or fractional sequence of the sequence of at least one of these polynucleotides and in particular (SEQ ID NO: 241, 195, 185 and 248), and
ii. capable of specifically inhibiting the correct transcription and/or translation of this polynucleotide.

30. A method for identifying a compound capable of inhibiting the extra-intestinal growth of E. coli, which comprises the detection of at least one compound capable of inhibiting the activity of a protein the orf of which comprises a polynucleotide such as used in the method of claim 29.

31. Kits for implementing a method according to anyone of claims 15 to 18, or the use according to claims 26 to 28 comprising at least a polynucleotide of the library according to anyone of claims 1 to 3.

32. The kits of claim 31, comprising at least one of the pairs of primers (SEQ ID NO: 160, 161), (SEQ ID NO: 162, 163) and (SEQ ID NO: 164, 165).

33. A vaccinal composition for preventing, alleviating or combatting an extra-intestinal development of E. coli, comprising an immunogenic polynucleotide such as isolated from the library according to anyone of claims 1 to 3 or an inactivated isogenic mutant thereof as active principle, or the corresponding ORFS in association with an inert carrier.

34. The vaccinal composition of claim 33, wherein said polynucleotide is polynucleotide selected from the group comprising:
the novel polynucleotides which are of novel nature B2/D+ A− according to the invention, i.e.
the sequences SEQ ID NO: 1 to NO: 153,
the polynucleotide sequences which can be obtained by digesting the total DNA of an E. coli of group B2 or group D (such as, for example, an E. coli strain isolated from the blood of a patient suffering from a septicaemic or meningeal infection) with a restriction enzyme chosen from the group consisting of NotI and BlnI, selecting those of the fragments obtained which hybridize with at least one sequence chosen from the group consisting of SEQ ID NO: 134, 144, 109, 115, 140, 135, 33, 56, 122, 130, 141, 25, 48, 51, 57, 121, 44, 45, 113, 119, 120, 23, 52 and sequencing selected fragments, and
the sequences of the orfs (open reading frames) which contain one of these sequences,
the nature-conserving variant or fractional sequences of these sequences.
the polynucleotides corresponding to the chuA gene and to its operon,
the polynucleotides the sequence of which corresponds to SEQ ID NO: 170, 171, 174, 175, 178, 179, 183, 185, 186, 190, 191, 193, 194, 195, 196, 199, 200, 202, 205, 206, 208, 209, 211, 214, 218, 220, 221, 233, 234, 235, 241, 244, 246, 247, 248 and 250,
the polynucleotides which are as obtained by digesting the total DNA of an E. coli of group B2 or D with a restriction enzyme chosen from the group consisting of NotI and BlnI, and selecting those of the fragments obtained which hybridize to at least one sequence chosen from the group consisting of SEQ ID NO: 125, 123, 116, 43, 40, 127, 133, 27, 34, 36, 42, 46, 54, 55, 38, 128 and 151, with the exception of the polynucleotides sfa, hly, cnf1, pap/prs, hra and ibe 10,
the polynucleotides the sequence of which corresponds to an orf (open reading frame) comprising the sequence of one of these polynucleotides,
the polynucleotides the sequence of which corresponds to a nature-conserving variant or fractional sequence of the sequence of at least one of these polynucleotides.

35. The vaccinal composition of claim 34, wherein said polynucleotide is selected from the group comprising SEQ ID NO 71, 114, 13, 77, 8, 36, 120, 130, 174, 202, 178, 221, 206, 175, 196, 170.
